(19) United States
(12) Patent Application Publication
(10) Pub. No.: US 2024/0084386 A1
(43) Pub. Date: Mar. 14, 2024

(54) MARKERS AND CELLULAR ANTECEDENTS OF RHEUMATOID ARTHRITIS FLARES

(71) Applicant: The Rockefeller University, New York, NY (US)

(72) Inventors: Robert B. Darnell, New York, NY (US); Dana Orange, New York, NY (US)

(21) Appl. No.: 17/994,090

(22) Filed: Nov. 25, 2022

Related U.S. Application Data
(60) Provisional application No. 63/283,359, filed on Nov. 26, 2021.

(51) Int. Cl. C12Q 1/6883; C12Q 1/6844

(52) U.S. Cl. CPC C12Q 1/6883; C12Q 1/6844; C12Q 2600/112; C12Q 2600/158

(57) ABSTRACT

The present disclosure provides biological markers which are molecular and cellular antecedents of rheumatoid arthritis (RA) flares. The present disclosure provides RNA and protein markers that can predict an RA flare one or two weeks prior to the flare. The present disclosure further provides blood circulating cells, particularly pre-inflammatory mesenchymal cells, which are cellular precursors and indicators of an impending RA flare. The present disclosure further provides methods, kits and markers for identification and monitoring of flares in RA patients and their application as markers and targets in and for treatment of rheumatoid arthritis and conditions induced or related to rheumatoid arthritis.

### CROSS REFERENCE TO RELATED APPLICATION

The present application claims priority to U.S. Application Ser. No. 63/283,359, filed Nov. 26, 2021, the entire contents of which is incorporated by reference herein.

### STATEMENT OF GOVERNMENT RIGHTS

This invention was made with government support under numbers NS034389, NS081706, NS097404 and 1UM1HG008901 awarded by the National Institutes of Health. The government has certain rights in the present disclosure.

### SEQUENCE LISTING

This application contains a Sequence Listing, which was submitted in XML format via EFS-Web, and is hereby incorporated by reference in its entirety. The XML copy, created on Nov. 22, 2022, is named “1119-75-PCT_ST26.xml” and is 37,645 bytes in size.

### FIELD

The present disclosure relates generally to the identification and characterization of biological markers which are molecular antecedents of rheumatoid arthritis (RA) flares. The present disclosure further relates to RNA and protein markers that can predict an RA flare one or two weeks prior to the flare. The present disclosure further relates to blood circulating cells, particularly pre-inflammatory mesenchymal cells, which are cellular precursors and indicators of an impending RA flare. The present disclosure relates to methods, kits and markers for the identification and monitoring of flares in RA patients and their application as markers and targets in and for treatment of rheumatoid arthritis and conditions induced or related to rheumatoid arthritis.

### BACKGROUND

Rheumatoid arthritis (RA) is a chronic inflammatory disorder and is the most common form of autoimmune arthritis, affecting more than 1.3 million Americans. About 75% of RA patients are women and between 1 and 3% of women may get rheumatoid arthritis in their lifetime. RA is a chronic disease affecting the lining of joints that causes joint pain, stiffness, swelling and decreased movement of the joints and can eventually result in bone erosion and joint deformity.

Treatments for RA can stop joint pain and swelling and prevent joint damage. Early treatment will give better long-term results; patients receiving early treatment are less likely to have the type of joint damage that leads to joint replacement. The main treatment goals with rheumatoid arthritis are to control inflammation, relieve pain, and reduce disability associated with RA. Treatment usually includes medications, occupational or physical therapy, and regular exercise, although some patients ultimately need surgery, such as synovectomy, tendon repair, joint fusion or total joint replacement to correct joint damage. Nonsteroidal anti-inflammatory drugs (NSAIDs) provide “first-line” RA medicines to relieve pain and reduce inflammation. NSAIDs include non-prescription drugs acetylsalicylate (aspirin), ibuprofen (Advil, Motrin IB) and naproxen sodium (Aleve, Naprosyn) and prescription NSAIDs, such as etodolac (Lodine) and diclofenac (Voltaren). Steroids are anti-inflammatory or immunosuppressants agents and they are prescribed for more severe RA or when RA symptoms flare to ease joint pain and stiffness. Examples of recognized steroids include glucocotricosteroids or corticosteroids, such as prednisone, cortisone and methylprednisolone. Disease-modifying antirheumatic drugs (DMARDs) are prescribed and utilized to slow the progression of RA and save joints and other tissues from permanent damage. Common DMARDs include methotrexate (Trexall, Otrexup), leflunomide (Arava), hydroxychloroquine (Plaquenil) and sulfasalazine (Azulfidine). DMARDs curb the overactive immune system in RA but they are not selective in their targets. Side effects vary but may include liver damage, bone marrow suppression and severe lung infections. Biologics—genetically engineered proteins which target a specific aspect or part of the immune system and act as immunosuppressants—are an increasingly important component in treatment of RA. Tumor necrosis factor (TNF) inhibitors and non-TNF inhibitors (such as cytokine inhibitors, T or B cell inhibitors) are included among the recognized biologics for RA. Biologics include abatacept (Orencia), adalimumab (Humira), anakinra (Kineret), baricitinib (Olumiant), certolizumab (Cimzia), etanercept (Enbrel), golimumab (Simponi), infliximab (Remicade), rituximab (Rituxan), sarilumab (Kevzara), tocilizumab (Actemra) and tofacitinib (Xeljanz). Adalimumab, etanercept, infliximab, golimumab and certolizumab target TNF (Lis K et al (2014) Arch Med Sci 10(6):1175-1185). Rituximab depletes B cells. Anakinra blocks the action of interleukin-1 (IL-1), a master cytokine. Abatacept targets T cells. These types of drugs also increase the risk of infections. Biologic DMARDs are usually most effective when paired with a nonbiologic DMARD, such as methotrexate. New drugs which are specific in their targets but are not biologics include oral small molecule Janus kinase (JAK) inhibitors such as tofacitinib (Xeljanz and Xeljanz XR), baricitinib (Olumiant), and upadacitinib (Rinvoq). The antimetabolite Methotrexate is also often used to treat RA, sometimes in combination with other DMARD drugs including biologic DMARDs.

Recent patient surveys indicate that three-fourths of RA patients are not satisfied with treatments and patients continued to experience bothersome symptoms that impacted their daily activities and life (Radawaski C et al (2019) Rheumatol Ther 6(3):461-471). RA, like many inflammatory diseases, is characterized by episodes of quiescence and exacerbation (flares). Flares are severe episodes of symptoms and reflect increased disease activity during which joint pain, swelling, and stiffness are more severe. The duration and intensity of flares vary, the flares are unpredictable, and the molecular events leading to flares are unknown. Such waxing/waning clinical courses are characteristic of many autoimmune diseases, including multiple sclerosis (MS) (Steinman L. (2014) Annu Rev Immunol 32:257-81), systemic lupus erythematosus (SLE) (Fava A, Petri M. (2019) J Autoimmun 96:1-13), and inflammatory bowel disease (IBD) (Braun J, Wei B (2007) Annu Rev Pathol 2:401-29; Braun J et al (2007) Arthritis Rheum 57:639-47.).

There remains a need for improved disease management among RA and other auto-immune disease patients. The present disclosure addresses such unmet needs in the field and particularly with regard to rheumatoid arthritis (RA).

The citation of references herein shall not be construed as an admission that such is prior art to the present disclosure.

### SUMMARY

In a general aspect, the present disclosure provides antecedents of an RA flare. RNA markers and protein markers have been identified, which are differentially expressed or preferentially expressed prior to an RA flare in an RA patient(s). These RNA transcripts provide markers that can predict an impending flare and the determination and presence of which can be utilized to implement and prescribe treatment and therapy to a patient(s).

In some embodiments, provided herein is a method for monitoring and/or predicting a rheumatoid arthritis (RA) flare or increased RA disease activity in a patient comprising: (a) detecting in the blood sample increased amounts of a panel of antecedent RA markers, wherein the panel comprises or consists of one or more AC3 markers listed in Table 10; (b) wherein the expression or quantitatively increased amounts of the one or more AC3 markers in the panel predicts an impending RA flare or increased RA disease activity.

In some embodiments, the panel of antecedent RA markers comprises or consists of one or more or all markers listed in Table 11. In some embodiments, the panel of antecedent RA markers comprises one or more or all markers listed in Table 12. In some embodiments, the panel of antecedent RA markers comprises or consists of one or more or all of the markers listed in Table 12 (COL14A1, DCLK1, FNDC1, COL16A1, COL1A2, KIAA1755, PXDN, and COL5A1).

In some embodiments, the panel of antecedent RA markers comprises or consists of at least 2 or more markers listed in Table 12 (COL14A1, DCLK1, FNDC1, COL16A1, COL1A2, KIAA1755, PXDN, and COL5A1).

In some embodiments, the panel of antecedent RA markers comprising or consisting of those listed in Table 12 (COL14A1, DCLK1, FNDC1, COL16A1, COL1A2, KIAA1755, PXDN, and COL5A1) are evaluated to detect increased amounts of one or more of the AC3 markers listed in Table 12 (COL14A1, DCLK1, FNDC1, COL16A1, COL1A2, KIAA1755, PXDN, and COL5A1).

In some embodiments, the panel of one or more antecedent RA markers comprising or consisting of those listed in Table 12 (COL14A1, DCLK1, FNDC1, COL16A1, COL1A2, KIAA1755, PXDN, and COL5A1) are evaluated utilizing primer pairs provided and listed in Table 13. In some embodiments, the panel of one or more antecedent RA markers comprising or consisting of those listed in Table 12 (COL14A1, DCLK1, FNDC1, COL16A1, COL1A2, KIAA1755, PXDN, and COL5A1) are evaluated utilizing one or more primer pairs selected from SEQ ID Nos: 1-28. In some embodiments, the panel of two or more antecedent RA markers comprising those listed in Table 12 (COL14A1, DCLK1, FNDC1, COL16A1, COL1A2, KIAA1755, PXDN, and COL5A1) are evaluated utilizing two or more primer pairs selected from SEQ ID Nos: 1-28. In some embodiments, the panel of antecedent RA markers comprising or consisting of those listed in Table 12 (COL14A1, DCLK1, FNDC1, COL16A1, COL1A2, KIAA1755, PXDN, and COL5A1) are evaluated utilizing applicable primer pairs selected from SEQ ID Nos: 1-28. In some embodiments, the increased amounts of the panel of AC3 RNA markers or the AC3 protein markers predict an RA flare in about 1 week or about 5-7 days or up to 2 weeks. In some embodiments, the method of any one of the preceding claims, wherein the panel consists of 2 to 283 antecedent markers. IN some embodiments, a panel of at least 3, at least 4, at least 5 or at least 6 of the AC3 markers are evaluated in any one of the methods disclosed herein. In some embodiments, the increased amounts of one or more RA antecedent RNA markers (e.g., AC3 RNA markers) are detected using RNAseq or RT-PCR. In some embodiments, the amount of antecedent AC3 markers are decreased in peripheral blood during an RA flare or once a patient exhibits symptoms of an RA flare.

In some embodiments, the method further comprises administering a therapeutically effective amount of one or more disease-modifying agents for treating RA if the amounts of the panel of the markers in the blood sample is increased relative to the amounts of the panel of the markers in the control blood sample. In some embodiments, this disclosure provides a collection of primer pairs for amplifying the antecedent RA markers in a panel disclosed herein. In some embodiments, the collection comprises one or more primer pairs in Table 13. In some embodiments, the collection of primer pairs comprises one or more of the following: i) a primer pair for amplifying COL14A1, wherein the primer pair comprises oligonucleotides having sequences of SEQ ID NO: 17 and SEQ ID NO: 18; ii) a primer pair for amplifying DCLK1, wherein the primer pair comprises oligonucleotides having sequences of SEQ ID NO: 19 and SEQ ID NO: 20; iii) a primer pair for amplifying FNDC1, wherein the primer pair comprises oligonucleotides having sequences of SEQ ID NO: 21 and SEQ ID NO: 22; iv) a primer pair for amplifying COL16A1, wherein the primer pair comprises oligonucleotides having sequences of SEQ ID NO: 23 and SEQ ID NO: 24; and (v) a primer pair for amplifying COL1A2, wherein the primer pair comprise oligonucleotides having sequences of SEQ ID NO: 25 and SEQ ID NO: 26.

In some embodiments, the collection of primer pairs comprises or consists of one or more of the following: i) a primer pair for amplifying COL14A1, wherein the primer pair comprise oligonucleotides having sequences of SEQ ID NO: 1 and SEQ ID NO: 2 or of SEQ ID NO: 3 and SEQ ID NO: 4; ii) a primer pair for amplifying DCLK1, wherein the primer pair comprise oligonucleotides having sequences of SEQ ID NO: 5 and SEQ ID NO: 6 or of SEQ ID NO: 7 and SEQ ID NO: 8; iii) a primer pair for amplifying FNDC1, wherein the primer pair comprise oligonucleotides having sequences of SEQ ID NO: 9 and SEQ ID NO: 10 or of SEQ ID NO: 11 and SEQ ID NO: 12; iv) a primer pair for amplifying COL16A1, wherein the primer pair comprise oligonucleotides having sequences of SEQ ID NO: 13 and SEQ ID NO: 14 or of SEQ ID NO: 15 and SEQ ID NO: 16; and v) a primer pair for amplifying COL1A2, wherein the primer pair comprise oligonucleotides having sequences of SEQ ID NO: 17 and SEQ ID NO: 18 or of SEQ ID NO: 19 and SEQ ID NO: 20.

In some embodiments, the collection of primer pairs comprise or consists of one or more of the following: i) a primer pair for amplifying COL14A1, wherein the primer pair comprise oligonucleotides having sequences of SEQ ID NO: 1 and SEQ ID NO: 2 or of SEQ ID NO: 3 and SEQ ID NO: 4; ii) a primer pair for amplifying DCLK1, wherein the primer pair comprise oligonucleotides having sequences of SEQ ID NO: 5 and SEQ ID NO: 6 or of SEQ ID NO: 7 and SEQ ID NO: 8; iii) a primer pair for amplifying FNDC1, wherein the primer pair comprise oligonucleotides having sequences of SEQ ID NO: 9 and SEQ ID NO: 10 or of SEQ ID NO: 11 and SEQ ID NO: 12; iv) a primer pair for amplifying COL16A1, wherein the primer pair comprise oligonucleotides having sequences of SEQ ID NO: 13 and SEQ ID NO: 14 or of SEQ ID NO: 15 and SEQ ID NO: 16; v) a primer pair for amplifying COL1A2, wherein the primer pair comprise oligonucleotides having sequences of SEQ ID NO: 17 and SEQ ID NO: 18 or of SEQ ID NO: 19 and SEQ ID NO: 20; (vi) a primer pair for amplifying KIAA1755, wherein the primer pair comprise oligonucleotides having sequences of SEQ ID NO: 21 and SEQ ID NO: 22; (vii) a primer pair for amplifying PXDN, wherein the primer pair comprise oligonucleotides having sequences of SEQ ID NO: 23 and SEQ ID NO: 24; and (viii) a primer pair for amplifying COL5A1, wherein the primer pair comprise oligonucleotides having sequences of SEQ ID NO: 25 and SEQ ID NO: 26 or of SEQ ID NO: 27 and SEQ ID NO: 28.

The present disclosure provides a method for monitoring and/or predicting a rheumatoid arthritis (RA) flare or increased RA disease activity in a patient comprising:
(a) evaluating a blood sample from said patient;
(b) evaluating the blood sample for expression or quantitatively increased amounts of one or more sets of antecedent RNA markers, protein markers or cell markers selected from:
(i) AC2 markers or proteins as provided in Table 7;
(ii) AC3 markers or proteins as provided in Table 8, 10, 11 or 12;
(iii) markers or proteins selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 as set out in Table 5;
(iv) markers or proteins selected from COL1A2, COL5A1, COL16A1, COL14A1, COL4A2, PXDN, ST5, DCLK1, SCARA5, EGFR, EGR1 and ZFHX4 as set out in Table 9;
(v) markers or proteins selected from COL14A1, DCLK1, FNDC1, COL16A1, COL1A2, KIAA1755, PXDN, and COL5A1 as set out in Table 12;
(vi) cell markers CD45− CD31−PDPN+;
(c) wherein the expression or quantitatively increased amounts of the RNA markers or proteins or the presence of the cell markers predicts an impending RA flare.

In some embodiments, the method does not include the step of collecting the blood from the patient.

The present disclosure provides a method for monitoring and/or predicting a rheumatoid arthritis (RA) flare in a patient comprising:
(a) isolating a blood sample from said patient;
(b) evaluating the blood sample for expression or quantitatively increased amounts of one or more sets of antecedent RNA markers, protein markers or cell markers selected from:
(i) markers or proteins selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 as set out in Table 5;
(ii) markers or proteins selected from COL1A2, COL5A1, COL16A1, COL14A1, COL4A2, PXDN, ST5, DCLK1, SCARA5, EGFR, EGR1 and ZFHX4 as set out in Table 9;
(iii) markers or proteins selected from COL14A1, DCLK1, FNDC1, COL16A1, COL1A2, KIAA1755, PXDN, and COL5A1 as set out in Table 12;
(iv) AC2 markers or proteins as provided in Table 7;
(v) AC3 markers or proteins as provided in Table 8, 10, 11 or 12; and
(vi) cell markers CD45− CD31−PDPN+;
(c) wherein the expression or quantitatively increased amounts of the RNA markers or proteins or the presence of the cell markers predicts an impending RA flare.

In an embodiment of the method, the expression or quantitatively increased amounts of the AC2 RNA markers or proteins predicts an RA flare in about 2 weeks or about 12-14 days. In an embodiment of the method, the expression or quantitatively increased amounts of the AC2 RNA markers or proteins predicts an RA flare in about 2 weeks, with a margin of error of about a week or further 7 days, thus in 7-21 days, or in up or about three weeks, up to 21 days or so.

In an embodiment of the method, the expression or quantitatively increased amounts of the AC3 RNA markers or proteins predicts an RA flare in about 1 week or about 5-7 days. In an embodiment of the method, the expression or quantitatively increased amounts of the AC3 RNA markers or proteins predicts an RA flare in about 1 week, with a margin of error of about a week or further 7 days, thus in 0-14 days, or in up or about two weeks, up to 14 days or so.

In an embodiment of the method, a panel of at least 20 of the AC2 or AC3 markers are evaluated. In an embodiment, a panel of at least 10 of the AC2 or AC3 markers are evaluated. At least 4, at least 5, at least 6, at least 7, at least 8, at least 9, at least 10, at least 11, at least 12, at least 13, at least 14, least 15, at least 16, at least 17, at least 18, at least 19, at least 20, at least 25, at least 30, at least 35, at least 40, at least 45, at least 50 of the AC2 or AC3 markers may be evaluated.

In an embodiment of the method, a panel of at least 20 of the AC2 and at least 20 of the AC3 markers are evaluated. In an embodiment, a panel of at least 10 of the AC2 and the AC3 markers are evaluated. At least 4, at least 5, at least 6, at least 7, at least 8, at least 9, at least 10, at least 11, at least 12, at least 13, at least 14, least 15, at least 16, at least 17, at least 18, at least 19, at least 20, at least 25, at least 30, at least 35, at least 40, at least 45, at least 50 of the AC2 and the AC3 markers may evaluated.

In an embodiment, a panel of at least two, three, four, five, six or seven markers selected from COL14A1, DCLK1, FNDC1, COL16A1, COL1A2, KIAA1755, PXDN, and COL5A1 as set out in Table 12 may be evaluated. In an embodiment, a panel of at least two markers selected from COL14A1, DCLK1, FNDC1, COL16A1, COL1A2, KIAA1755, PXDN, and COL5A1 as set out in Table 12 may be evaluated. In an embodiment, a panel of at least three markers selected from COL14A1, DCLK1, FNDC1, COL16A1, COL1A2, KIAA1755, PXDN, and COL5A1 as set out in Table 12 may be evaluated. In an embodiment, a panel of at least four markers selected from COL14A1, DCLK1, FNDC1, COL16A1, COL1A2, KIAA1755, PXDN, and COL5A1 as set out in Table 12 may be evaluated. In an embodiment, a panel of at least five markers selected from COL14A1, DCLK1, FNDC1, COL16A1, COL1A2, KIAA1755, PXDN, and COL5A1 as set out in Table 12 may be evaluated. In an embodiment, a panel of at least six markers selected from COL14A1, DCLK1, FNDC1, COL16A1, COL1A2, KIAA1755, PXDN, and COL5A1 as set out in Table 12 may be evaluated. In an embodiment, a panel of at least seven markers selected from COL14A1, DCLK1, FNDC1, COL16A1, COL1A2, KIAA1755, PXDN, and COL5A1 as set out in Table 12 may be evaluated. In an embodiment, a panel of markers COL14A1, DCLK1, FNDC1, COL16A1, COL1A2, KIAA1755, PXDN, and COL5A1 as set out in Table 12 may be evaluated.

In an embodiment, sublining fibroblast markers selected from the AC3 markers or proteins are evaluated. In an embodiment, AC3 markers or proteins expressed by CD34+, HLADR+ and DKK3+ cells are evaluated. In an embodiment, AC3 markers or proteins expressed by CD45−, CD34+, HLADR+ and DKK3+ cells are evaluated.

The method includes, wherein the cell marker IL-17RD is also evaluated.

The present disclosure further provides a method(s) wherein the antecedent RNA markers or protein markers or selected from:
(a) AC3 markers or proteins as provided in Table 8, 10, 11 or 12;
(b) markers or proteins selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 as set out in Table 5;
(c) markers or proteins selected from COL1A2, COL5A1, COL16A1, COL14A1, COL4A2, PXDN, ST5, DCLK1, SCARA5, EGFR, EGR1 and ZFHX4 as set out in Table 9; and
(d) markers or proteins selected from COL14A1, DCLK1, FNDC1, COL16A1, COL1A2, KIAA1755, PXDN, and COL5A1 as set out in Table 12;
   are decreased in peripheral blood during an RA flare or once a patient exhibits symptoms of an RA flare.

The present disclosure further provides a method(s) wherein the antecedent RNA markers or protein markers or selected from:
(a) markers or proteins selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 as set out in Table 5;
(b) markers or proteins selected from COL1A2, COL5A1, COL16A1, COL14A1, COL4A2, PXDN, ST5, DCLK1, SCARA5, EGFR, EGR1 and ZFHX4 as set out in Table 9;
(c) AC3 markers or proteins as provided in Table 8, 10 or 11; and
(d) AC3 markers or proteins selected from COL14A1, DCLK1, FNDC1, COL16A1, COL1A2, KIAA1755, PXDN, and COL5A1 as set out in Table 12;
   are decreased in peripheral blood during an RA flare or once a patient exhibits symptoms of an RA flare.

The present disclosure provides a method for predicting an impending RA flare and treating a flare in a patient, the method comprising:
(a) contacting a blood sample from the patient with reagents specific for markers selected from a panel of RNA or protein markers to assess expression of the RNA or protein markers, wherein the panel of RNA or protein markers is selected from:
(i) AC2 markers or proteins as provided in Table 7;
(ii) AC3 markers or proteins as provided in Table 8, 10 or 11;
(iii) markers or proteins selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 as set out in Table 5;
(iv) markers or proteins selected from COL1A2, COL5A1, COL16A1, COL14A1, COL4A2, PXDN, ST5, DCLK1, SCARA5, EGFR, EGR1 and ZFHX4 as set out in Table 9; and
(v) markers or proteins selected from COL14A1, DCLK1, FNDC1, COL16A1, COL1A2, KIAA1755, PXDN, and COL5A1 as set out in Table 12;
(b) comparing expression of the markers selected from a panel of RNA or protein markers in the blood sample to expression of the markers in a control blood sample to determine if expression of the markers selected from a panel of RNA or protein markers in the blood sample is increased relative to expression in the control blood sample, wherein detection of increased expression serves to predict an impending RA flare in a patient;
(c) and treating the patient thereby diagnosed with an impending RA flare by administering a therapeutically effective amount of one or more disease-modifying agent for treating RA. As used herein, the term “disease-modifying agent” refers to that an agent that is capable of alleviating the symptoms of RA when administered to a patient who suffers from RA.

The present disclosure provides a method for predicting an impending RA flare and treating a flare in a patient, the method comprising:
(a) contacting a blood sample from the patient with reagents specific for markers selected from a panel of RNA or protein markers to assess expression of the RNA or protein markers, wherein the panel of RNA or protein markers is selected from:
(i) markers or proteins selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 as set out in Table 5;
(ii) markers or proteins selected from COL1A2, COL5A1, COL16A1, COL14A1, COL4A2, PXDN, ST5, DCLK1, SCARA5, EGFR, EGR1 and ZFHX4 as set out in Table 9;
(iii) AC2 markers or proteins as provided in Table 7;
(iv) AC3 markers or proteins as provided in Table 8, 10 or 11; and
(v) AC3 markers or proteins selected from COL14A1, DCLK1, FNDC1, COL16A1, COL1A2, KIAA1755, PXDN, and COL5A1 as set out in Table 12; and
(b) comparing expression of the markers selected from a panel of RNA or protein markers in the blood sample to expression of the respective markers in a control blood sample to determine if expression of the markers selected from a panel of RNA or protein markers in the blood sample is increased relative to expression in the control blood sample, wherein detection of increased expression serves to predict an impending RA flare in a patient; and treating the patient diagnosed with an impending RA flare by administering a therapeutically effective amount of one or more disease-modifying agent for treating RA.

In an embodiment of the method, the expression, differential expression, or quantitatively increased amounts of the AC2 RNA markers or proteins predicts an RA flare in about 2 weeks, about 14 days, or about 12-14 days.

In an embodiment of the method, the expression or quantitatively increased amounts of the AC3 RNA markers or proteins predicts an RA flare in about one week, about 7 days, or about 5-7 days. In an embodiment of the method, the expression, differential expression, or quantitatively increased amounts of the markers or proteins selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 or of the markers or proteins selected from COL1A2, COL5A1, COL16A1, COL14A1, COL4A2, PXDN, ST5, DCLK1, SCARA5, EGFR, EGR1 and ZFHX4 predicts an RA flare in about one week, about 7 days, or about 5-7 days.

In an embodiment of the method, the expression or quantitatively increased amounts of the AC3 RNA markers or proteins predicts an RA flare in about one week, about 7 days, or about 5-7 days. In an embodiment of the method, the expression, differential expression, or quantitatively increased amounts of the markers or proteins selected from COL14A1, DCLK1, FNDC1, COL16A1, COL1A2, KIAA1755, PXDN, and COL5A1 as set out in Table 12 predicts an RA flare in about one week, about 7 days, or about 5-7 days.

In an embodiment of the method, the expression or quantitatively increased amounts of RNA or protein markers selected from:
(a) the AC3 RNA markers or proteins;
(b) markers or proteins selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6;
(c) markers or proteins selected from COL1A2, COL5A1, COL16A1, COL14A1, COL4A2, PXDN, ST5, DCLK1, SCARA5, EGFR, EGR1 and ZFHX4; and
(d) markers or proteins selected from COL14A1, DCLK1, FNDC1, COL16A1, COL1A2, KIAA1755, PXDN, and COL5A1 as set out in Table 12; predicts an RA flare in about 1 week or about 5-7 days.

In an embodiment of the method, the expression or quantitatively increased amounts of RNA or protein markers selected from:
(a) markers or proteins selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6;
(b) markers or proteins selected from COL1A2, COL5A1, COL16A1, COL14A1, COL4A2, PXDN, ST5, DCLK1, SCARA5, EGFR, EGR1 and ZFHX4;
(c) markers or proteins selected from COL14A1, DCLK1, FNDC1, COL16A1, COL1A2, KIAA1755, PXDN, and COL5A1 as set out in Table 12; and
(d) the AC3 RNA markers or proteins; predicts an RA flare in about 1 week or about 5-7 days.

Evaluation of RNA or protein expression may be evaluated or assessed using any method known in the art. Thus, in accordance with the methods of the present disclosure, RNA expression may be assessed by RT PCR. In accordance with the method, protein expression may be detected using specific antibodies.

Cell marker expression or presence on the surface of cells in the blood in accordance with the methods of the present disclosure may be determined using standard and known methods. Cell markers may be evaluated using antibodies. Cell markers may be evaluated using Fluorescent Activated Cell Sorting (FACs) analysis. Cells or cell markers may be evaluated using cell sorting or single-cell assessments.

In embodiments of the method of the present disclosure, a patient is treated with a disease-modifying agent for treating RA after a patient is diagnosed with an impending RA flare using a method disclosed herein. In some embodiments, the disease-modifying agent may be selected from standard or clinically recognized agents or therapies for RA or arthritic conditions or inflammatory diseases and conditions. In embodiments of the method of the present disclosure, the disease-modifying agent for treating RA may be one or more agent selected from a nonsteroidal anti-inflammatory drug (NSAID), steroid, methotrexate, disease-modifying antirheumatic drug (DMARDs), biologic DMARD, and oral janus kinase (JAK) inhibitor. In an embodiment, the DMARD is selected from methotrexate (Trexall, Otrexup), leflunomide (Arava), hydroxychloroquine (Plaquenil) and sulfasalazine (Azulfidine). There are a variety of known biologic DMARD agents, including various agents being evaluated or with application to RA and/or other arthritic and/or inflammatory conditions. In an aspect, the biologic DMARD may selected from abatacept (Orencia), adalimumab (Humira), anakinra (Kineret), baricitinib (Olumiant), certolizumab (Cimzia), etanercept (Enbrel), golimumab (Simponi), infliximab (Remicade), rituximab (Rituxan), sarilumab (Kevzara), tocilizumab (Actemra) and tofacitinib (Xeljanz). The biologic DMARD may be a tumor necrosis factor (TNF) inhibitor. In an aspect, the biologic DMARD may be an anti-inflammatory antibody, or an antibody directed to an inflammation or immune modulatory molecule. In an aspect, the antibody may be an interleukin antibody. In some embodiments, the antibody is an IL-17 specific antibody or an IL-17RD specific or IL-17RD blocking or neutralizing antibody. In some embodiments, the IL-17 specific antibody targets one or more members of the IL-17 family selected from the group consisting of IL-17A, IL-17B, IL-17C, IL-17D, IL-17E/IL-25, and IL-17F. In some embodiments, the antibody targets one or more members of the IL-17 receptor family selected from the group consisting of IL-17RA, IL-17RB, IL-17RC, IL-17RD and IL-17RE. In one embodiment, the antibody is netakimab.

In one embodiment, the biologic DMARD is combined with an NSAID and/or with methotrexate. In an embodiment, the JAK inhibitor is selected from tofacitinib (Xeljanz and Xeljanz XR), (Olumiant), and upadacitinib (Rinvoq).

The present disclosure provides and relates to a circulating pre-inflammatory mesenchymal (PRIME) cell characterized as a CD45−CD31−PDPN+ cell, wherein the presence of the cell in peripheral blood is indicative or predictive of an impending RA flare. In an embodiment, the PRIME cell additionally expresses IL-17RD and is IL-17RD+. In an embodiment, a plurality of PRIME cells additionally expresses IL-17RD and is IL-17RD+.

The present disclosure further provides a method of predicting an impending RA flare comprising evaluating a blood sample from a patient for the presence of a PRIME cell characterized as a CD45−CD31−PDPN+ cell, wherein the presence of detectable PRIME cells in peripheral blood in a patient predicts an impending RA flare in the patient. In an embodiment thereof, the method includes further evaluating for the presence of IL-17RD on a CD45−CD31−PDPN+ cell.

Methods are provided for evaluating and treating an impending flare in an RA patient comprising evaluating the peripheral blood of a patient for the presence of a PRIME cell characterized as a CD45−CD31−PDPN+ cell, and treating a patient that is positive for PRIME cells in their peripheral blood with a disease-modifying agent for RA. Methods are provided for evaluating and treating an impending flare in an RA patient comprising evaluating the peripheral blood of a patient for the presence of a PRIME cell characterized as a CD45−CD31−PDPN+IL-17RD+ cell and treating a patient that is positive for PRIME cells in their peripheral blood with a disease-modifying agent for RA.

In an additional embodiment of the method, the patient is treated with an IL-17 or IL-17RD antibody. In another embodiment, the patient is further treated with an anti-inflammatory agent and/or an immune modulating agent.

Methods are provided for evaluating and treating an impending flare in an RA patient comprising evaluating the peripheral blood of a patient for the presence of RNA(s) or protein(s) expressed, specifically expressed or particularly expressed by a PRIME cell characterized as a CD45−CD31−PDPN+ cell and treating a patient that is positive for expressing, specifically expressing or particularly expressing RNA(s) or protein(s) of PRIME cells in their peripheral blood with a disease-modifying agent for RA. Methods are provided for evaluating and treating an impending flare in an RA patient comprising evaluating the peripheral blood of a patient for the presence of RNA(s) or protein(s) expressed, specifically expressed or particularly expressed by a cell characterized as a CD45−CD31−PDPN+IL-17RD+ cell and treating a patient that is positive for the presence of RNA(s) or protein(s) expressed, specifically expressed or particularly expressed by a cell characterized as a CD45−CD31−PDPN+IL-17RD+ cell in their peripheral blood with a disease-modifying agent for RA.

The present disclosure includes a set of RNA or protein markers for evaluating and predicting an impending RA flare in a patient comprising or consisting of the markers selected from the group consisting of:
(i) a panel of at least 20 markers from the AC2 markers provided in Table 7;
(ii) a panel of at least 20 markers from the AC3 markers provided in Table 8, 10 or 11;
(iii) markers selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 as set out in Table 5;
(iv) markers selected from COL1A2, COL5A1, COL16A1, COL14A1, COL4A2, PXDN, ST5, DCLK1, SCARA5, EGFR, EGR1 and ZFHX4 as set out in Table 9; and
(v) markers selected from COL14A1, DCLK1, FNDC1, COL16A1, COL1A2, KIAA1755, PXDN, and COL5A1 as set out in Table 12.

In an aspect of the method, a panel of at least 20 of the AC2 or AC3 markers are provided. In an aspect, a panel of at least 10 of the AC2 or AC3 markers are provided. At least 4, at least 5, at least 6, at least 7, at least 8, at least 9, at least 10, at least 11, at least 12, at least 13, at least 14, least 15, at least 16, at least 17, at least 18, at least 19, at least 20, at least 25, at least 30, at least 35, at least 40, at least 45, at least 50 of the AC2 or AC3 markers may provided.

In an embodiment, the panel of AC2 markers comprises naïve B cell gene markers and markers of developmental pathways for naïve B cells and leukocytes. In an embodiment, wherein the panel of AC3 markers comprises markers of cartilage morphogenesis, endochondral bone growth, extracellular matrix organization and sublining fibroblasts.

In another embodiment of the present disclosure a set of one or more markers selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 is provided and/or utilized in accordance with the methods hereof. In an aspect, a set of one or more markers selected from COL1A2, COL5A1, COL16A1, COL14A1, COL4A2, PXDN, ST5, DCLK1, SCARA5, EGFR, EGR1 and ZFHX4 is provided and/or utilized in accordance with the methods hereof. In another embodiment, a set of one or markers selected from COL14A1, DCLK1, FNDC1, COL16A1, COL1A2, KIAA1755, PXDN, and COL5A1 as set out in Table 12 is provided and/or utilized in accordance with the methods hereof. In an aspect, a set of two or more, three or more, four or more, five or more, six or more, seven or more, eight or more, nine or more, ten or more, a dozen, at least two, at least three, at least four, at least five, at least six, at least seven, at least eight, at least nine, at least ten, a set of 5-10, a set of 3-5, a set of 5-7, a set of 3-7, a set of 5-8 selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6, a set of 2, 3, 4, 5, 6, 7 of or all of COL14A1, DCLK1, FNDC1, COL16A1, COL1A2, KIAA1755, PXDN, and COL5A1 as set out in Table 12 is provided and/or utilized in accordance with the methods hereof. In an aspect, a set of two or more, three or more, four or more, five or more, six or more, seven or more, eight or more, nine or more, ten or more, a dozen, at least two, at least three, at least four, at least five, at least six, at least seven, at least eight, at least nine, at least ten, a set of 5-10, a set of 3-5, a set of 5-7, a set of 3-7, a set of 5-8 selected from COL1A2, COL5A1, COL16A1, COL14A1, COL4A2, PXDN, ST5, DCLK1, SCARA5, EGFR, EGR1 and ZFHX4 is provided and/or utilized in accordance with the methods hereof. In an aspect, a set of two or more, three or more, four or more, five or more, six or more, seven or more, at least two, at least three, at least four, at least five, at least six, at least seven selected from, or all of COL14A1, DCLK1, FNDC1, COL16A1, COL1A2, KIAA1755, PXDN, and COL5A1 as set out in Table 12 is provided and/or utilized in accordance with the methods hereof.

The present disclosure also provides a system or kit for predicting an impending RA flare comprising a set of markers as described herein or a set of probes and/or antibodies for evaluating a set of markers as described herein. As an example, a kit or system may include a set of markers or a set of probes and/or antibodies for evaluating a set of markers selected from or for a or any combination of:
(i) a panel of at least 20 markers from the AC2 markers provided in Table 7;
(ii) a panel of at least 20 markers from the AC3 markers provided in Table 8, 10 or 11;
(iii) markers selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 as set out in Table 5;
(iv) markers selected from COL1A2, COL5A1, COL16A1, COL14A1, COL4A2, PXDN, ST5, DCLK1, SCARA5, EGFR, EGR1 and ZFHX4 as set out in Table 9;
(v) markers selected from COL14A1, DCLK1, FNDC1, COL16A1, COL1A2, KIAA1755, PXDN, and COL5A1 as set out in Table 12.

In some embodiments, provided herein is a collection of primer pairs for amplifying the AC3 markers in Table 12, wherein the collection of primers comprises or consists of one or more of the primers listed in Table 13. In some embodiments, provided herein is a collection of primer pairs for amplifying the AC3 markers in Table 12, wherein the collection of primers comprise or consists of one or more of the primers SEQ ID Nos: 1-28. In some embodiments, provided herein is a collection of primer pairs for amplifying the AC3 markers in Table 12, wherein the collection of primers comprise or consists of one or more of the forward and reverse primer pairs listed in Table 13. In some embodiments, provided herein is a system or kit for predicting an impending RA flare or increased RA disease activity comprising a panel of markers, wherein the markers are selected from one or more antecendent RA markers listed in Table 10, Table 11, or Table 12.

In some embodiments, a system or kit disclosed herein further comprises a means for collecting the patient's blood by fingerstick.

In some embodiments, provided herein is a computer-implemented method for predicting an impending RA flare or increased RA disease activity in a patient comprising: a) detecting amounts of a panel of AC3 markers, wherein the panel comprises or consists of one or more AC3 markers listed in Table 10, and b) determining, using one or more computer processors, that the patient has an impending RA flare or increased RA disease activity if the amounts of the panel of AC3 markers are higher than the amounts of the panel of AC3 markers in a control blood sample.

In some embodiments, provided herein is a computer-implemented method for monitoring and/or predicting an impending RA flare or increased RA disease activity in a patient comprising:
a) detecting amounts of a panel of AC3 markers, wherein the panel comprises or consists of one or more or all of the AC3 markers listed in Table 11, and b) determining, using one or more computer processors, that the patient has an impending RA flare or increased RA disease activity if the amounts of the panel of AC3 markers are higher than the amounts of the AC3 markers in a control blood sample.

In some embodiments, provided herein is a computer-implemented method for monitoring and/or predicting an impending RA flare or increased RA disease activity in a patient comprising:
a) detecting amounts of a panel of AC3 markers, wherein the panel comprises or consists of one or more or all of the AC3 markers listed in Table 12, and b) determining, using one or more computer processors, that the patient has an impending RA flare or increased RA disease activity if the amounts of the panel of AC3 markers are higher than the amounts of the AC3 markers in a control blood sample.

In some embodiments, any of the computer-implemented method for monitoring and/or predicting an impending RA flare or increased RA disease activity in a patient as disclosed above further comprises: c) comparing the amounts of the markers in the panel to the amounts of the markers in a control blood sample; and d) administering a therapeutically effective amount of one or more disease-modifying agent for treating RA if the amounts of the markers of the panel in the blood sample is increased relative to the amounts of the markers in the control blood sample, thereby treating the impending flare in the patient comparing the amounts of the markers in the panel to the amounts of the markers in a control blood sample. In some embodiments, step (d) is performed within one (1) week or within 5-7 days from the step (a) of contacting a blood sample from the patient with reagents specific for detecting a panel of AC3 markers of Table 10, 11, or 12.

In some embodiments, the increased amounts of the AC3 markers predicts an RA flare in about 1 week or about 5-7 days.

In some embodiments, the markers or marker panels used any one of the methods disclosed herein does not include one or more of Transforming growth factor beta 1 (TGFB1), Tissue inhibitor of metalloproteinase 1 (TIMP1), Interleukin 1 receptor antagonist (IL1RN), COL1A2, COL3A1, and Clusterin (CLU).

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

The patent or patent application contains at least one drawing executed in color. Copies of this patent or patent application publication with color drawing(s) will be provided by the U.S. Patent and Trademark Office upon request and payment of the necessary fee.

FIGS. 1A, 1B and 1C depict the study overview and validation of in-home assessments of disease activity and gene expression. FIG. 1A. Clinical data collection and RNA analysis over time. Study overview of clinical data and sample collection over time. FIG. 1B. Clinical and patient reported assessments of disease activity. Correlation between disease activity scores measured in clinic (DAS28) and at home (RAPID3 questionnaire) from the index patient. Locally Weighted Smoothed Scatterplots Showing the Relationship between the Change in RAPID3 scores and DAS28 in the index patient. The solid line represents the point estimates and the gray area represents the 95 percent confidence intervals.

FIG. 1C. Clinical blood counts and RNASeq-inferred blood counts. Neutrophil, lymphocyte, and monocyte counts measured from paired clinical complete blood counts from venipuncture blood draws and CIBERSORTx inferred blood counts from RNAseq data from fingerstick blood draws (N=38 paired samples).

FIGS. 2A, 2B, 2C and 2D provide clinical and transcriptional characteristics of RA flares in index patient. FIG. 2A. Index Patient disease activity over time. Disease activity (RAPID3 questionnaire, N=356), over the course of four years in index patient. Time points are colored according to disease activity category. FIG. 2B. Differential expression of genes in flare. Volcano plot of differential gene expression of flare (N=46) versus baseline (N=33), plotting statistical significance (−log 10(FDR)) against fold change (log 2(FC)) (gray points are non-significant genes, i.e., FDR>0.1, red indicates FDR<0.1 and log 2 fold change >0, blue indicates FDR<0.1 and log 2 fold change <0). Pathways enriched in significantly increased (FIG. 2C.) (Pathways increased in flare) or decreased genes (FIG. 2D.) (Pathways decreased in flare) in flare relative to baseline.

FIGS. 3A, 3B, 3C, 3D and 3E provide transcriptional characteristics of immune activation prior to symptom onset in RA flares. FIG. 3A. Disease activity scores over time to flare (measured in days). Box represents disease activity from day −56 to +28 over time to flare. Vertical arrows (in A-D) represent start of flare. FIG. 3B. Hierarchical clustering of z scores of 2791 significantly differentially expressed genes over time to flare. Statistically significant clusters are labeled by color. AC2 and AC3 refer to clusters that changed antecedent to flare. FIG. 3C. Detailed representation of cluster 1, antecedent cluster 2 (AC2), and antecedent cluster 3 (AC3) genes from FIG. 3B over time to flare. FIG. 3D. Mean standardized cluster gene expression over time to flare. Light grey lines represent expression of individual genes in the cluster. Dashed horizontal line represents mean baseline gene expression (weeks −8 to −4). Dashed vertical line represents start of flare. FIG. 3E. Pathways enriched in clusters 1, AC2, and AC3.

FIGS. 4A, 4B, 4C, 4D, 4E. PRIME cells express AC3 genes. FIG. 4A. Synovial cell subtype marker genes in clusters identified in blood (FIG. 3A). Enrichment scores of 200 single-cell RNAseq marker genes from 18 synovial panel cell types. Dashed line represents threshold for significance (FDR<0.05 or -log 10 FDR>1.3). FIG. 4B. Mean standardized gene expression and 95% confidence intervals of genes common to synovial sublining fibroblasts (CD34+, DKK+ and HLA-DRA+ fibroblasts) and AC3 in blood over time to flare (dashed vertical line represents start of flare). Error bars represent confidence intervals. FIG. 4C. Venn diagram of AC3 genes that decrease during flare in 4 patients. FIG. 4D. Flow cytometry of blood samples from 19 RA patients and 18 healthy volunteers (HV). Percent PDPN+/CD45− cells of TOPRO-(live)/CD31− cells is presented. P value represents result of two-sided t-test. FIG. 4E. Log ₂ fold change of AC3 genes expressed in PRIME cells (flow sorted CD45−/CD31−/PDPN+ cells) versus hematopoietic cells (flow sorted CD45+) and Log₂ fold change of input cells (stained PBMC but not flow sorted) versus hematopoietic cells (flow sorted CD45+) as technical control for stress of flow sorting.

FIG. 5 provides a model of blood and synovial gene expression changes antecedent to and during RA flares. Inflammatory signals activate naïve B cells (AC2; FIG. 3C-E), which in turn activate PRIME cells (AC3; FIG. 3C-E) which harbor the signature of synovial sublining fibroblast genes (FIG. 4A). The model proposes that PRIME cells demarginate and are increased in blood prior to flare and then decrease (FIG. 4B) just after symptom onset; these cells or their progeny are increased in inflammatory RA synovium where they contribute to and may be sufficient to cause joint inflammation.

FIG. 6 depicts RNA quality and quantity by volume of fixative. 3 drops of blood harvested with a 21 gauge lancet were added to a microtainer tube prefilled with either 250, 500 or 750 ul of PAX gene fixative. Samples were stored at room temperature for 3 days and then RNA was extracted using the PAXgene© RNA kit and RIN scores and quantity of RNA was assessed using the Agilent 2100 Bioanalyzer picochip. Padj=ANOVA, followed by Dunnett's multiple comparisons test, using 250 ul as the reference group.

FIG. 7 depicts RNA quality and quantity by time at room temperature. 100 ul of whole blood was added to a microtainer tube prefilled with 250 ul PAX gene fixative and frozen after 2 hours, 3 days, or 7 days incubation at room temperature. RNA was extracted using the PAXgene RNA kit with scaled down washes and elutions and RIN scores and quantity of RNA was assessed using the Agilent 2100 BioAnalyzer RNA picochip. Padj=ANOVA, followed by Dunnett's multiple comparisons test, using Day 0 as the reference group.

FIG. 8 depicts RNA quality and quantity of fresh and mailed samples. 100 ul of whole blood was added to a microtainer tube prefilled with 250 ul PAXgene fixative and frozen after two-hour incubation at room temperature or mailed. RNA was extracted using the PAXgene RNA kit and RIN scores and quantity of RNA was assessed using the Agilent 2100 BioAnalyzer RNA picochip.

FIG. 9 depicts RNA quality and quantity by volume of extraction and washes. 3 drops of blood harvested with a 21-gauge lancet were added to a microtainer tube prefilled with 250 ul of PAX gene fixative. Samples were stored at room temperature for 3 days and then RNA was extracted using the PAXgene© RNA kit according to manufacturer's directions or with a scaled down version of the PAX protocol, using 25% of the recommended volumes for all washes and elutions. RIN scores and quantity of RNA was assessed using the Agilent 2100 BioAnalyzerRNA picochip. P=unpaired two-sided t test.

FIG. 10 depicts RNA quality and quantity with and without TriZol reagent extraction step. Mailed patient fingerstick samples were stored in PAXgene® RNA buffer at −80° C. 142 samples had RNA extracted with PAXgene® RNA extraction with low volume washes, 13 samples were thawed and mixed with 700 ul Trizol-LS, and 250 ul chloroform. After centrifugation, the top layer was precipitated with isopropanol and glycogen and washed with 80% cold ethanol, centrifuged and the pellet was dried, resuspended in PBS and then purified using the Roche High Pure Isolation kit. P values represent significance of unpaired T tests.

FIG. 11 depicts Cycle Times for HbgA2, 18S RNA, and TNF alpha after GlobinZero depletion. Since ribosomal and hemoglobin RNA represent approximately 98% and 70% of the RNA in whole blood, respectively, we tested standard commercial kits for removing these RNAs prior to RNAseq. 4 ml heparinized blood, treated with 1 ug/ml LPS for one hour at 37° C. and placed 250 ul into 250 ul PAXgene fixative into replicate microtainer tubes. After RNA extraction samples were either left undepleted or treated with the globin zero depletion kit and then quantitative PCR was performed to test for hemoglobin A2, 18S RNA, or TNF alpha mRNA expression. GlobinZero kits depleted both hemoglobin A2 and 18S ribosomal RNA (increased mean cycle time from 11 to 28 and 10 to 30, respectively) with relative preservation of TNFalpha mRNA. P values represent results of ordinary one-way ANOVA with Tukey's multiple comparisons test.

FIG. 12 provides RNASeq QC metrics of RNA with various quality scores prepared with Illumina TruSeq or Kapa Hyper Prep Kits. A. (Left Panel): Distribution of mapping, uniquely mapping, and duplicate reads. B. (Right Panel):Distribution of tags assigned to UTR (untranslated region), intergenic, intronic, and CDS (coding sequence) of whole blood RNA samples prepared with Illumina TruSeq or Kapa Hyper Prep Kits with various input RNA quality and quantity. The Illumina TruSeq library Prep demonstrated increased mapping to coding sequence and fewer intergenic reads and was ultimately used for downstream experiments.

FIG. 13 provides comparison of patient reported (RAPID3) and clinical (DAS28) disease activity scores of 4 patients. Paired RAPID3 scores and DAS28-CRP scores were collected from 91 clinic visits of 4 RA patients. The patient reported RAPID3 questionnaire was significantly correlated with clinician generated DAS28 score in all 4 patients.

FIG. 14 depicts clinical features of baseline, flare and on steroid treatment. RAPID3 questionnaire responses from 360 time points and DAS28 ESR, TJC, SJC, ESR, DAS28 CRP, platelet counts and absolute neutrophil counts from 43 clinic visits for one patient over four years. Time points are positioned and colored according to disease activity category: The first (left) set in each graph is baseline, the middle set in each graph is flare, and the third (right) set in each graph is steroid. Steroid treatment was defined as any time point when the patient took any dose of steroid that day, or if the calculated dose, using washout kinetics, was greater than 0.01 mg/ml. Samples acquired between two time points that met criteria for flare that did not meet flare criteria were still categorized as flare up until treatment with steroid. TJC indicates tender joint count. SJC indicates swollen joint count. P values represent ANOVA across three disease categories. Flare was associated with significantly increased RAPID3, DAS28 ESR, TJC, SJC, ESR, DAS28CRP, platelets and neutrophils.

FIGS. 15A and 15B depict that differentially expressed flare genes are reproducibly altered in repeated flares. FIG. 15A. Index patient disease activity (RAPID3) over time. Top panel dots are colored by disease activity assignment. Bottom panel dots are colored according to clinical flare event number. FIG. 15B. Unsupervised hierarchical clustering of genes differentially expressed between baseline and flare. Top bar indicates samples colored according to disease activity assignment. Bottom bar indicates samples colored according to clinical flare event number. Data shows differentially expressed flare genes are represented by multiple clinical events.

FIGS. 16A and 16B provide deconvolution of blood cell types over time to flare. Mean cell type trajectories of FIG. 16A. ABIS inferred cell types, and FIG. 16B. CIBERSORTx inferred cell types over time to flare are plotted (excluding those that were 0 all throughout) showing the mean score with standard error of the mean as a ribbon. These data independently confirm data in main FIG. 4A identifying activation of B cells in AC2.

FIG. 17 depicts that distinct analysis approaches identify activation of naïve B cells in blood 2 weeks prior to flare. A. CIBERSORTx inferred naïve B cells by week to flare. B. ABIS inferred naïve B cells by week to flare. C. Mean expression of 190 synovial single-cell RNAseq naïve B cell marker genes by week to flare. D. IGHM (blue/top) and IGHD (red/bottom) gene expression by week to flare. Dashed line indicates first day of symptoms of RA flare, red arrows indicate peak in B cell signature 2 weeks prior to flare.

FIG. 18 provides mean standardized gene expression of genes common to synovial sublining fibroblasts (CD34+, DKK+, and HLA-DR+ fibroblasts) and AC3 in blood over time to flare. Light gray lines represent the expression of individual genes over time to flare in patient 1 over all flares.

FIG. 19 depicts gating strategy for quantification of PRIME cells in blood samples. Previously frozen peripheral blood mononuclear cells were thawed and stained with antibodies to CD31, PDPN, and CD45 as well as TOPRO. Live CD31− cells were gated and PDPN+, CD45− cells were enumerated.

FIG. 20 demonstrates PRIME cells are nucleated. PBMC of RA donor was stained with CD45/CD31/PDPN and either TOPRO (Not Permeabilized) or permeabilization and TOPRO (Permeabilized) and assessed by flow cytometry. PRIME cells were gated as CD45−/CD31−/PDPN+ cells and TOPRO staining of permeabilized and not permeabilized cells is presented. The increased fluorescence of TOPRO in the permeabilized PRIME cells indicates the presence of double stranded nucleic acid.

FIG. 21 depicts that sorted PRIME cells express synovial fibroblast genes. Log 2 fold change of various synovial single-cell RNAseq marker genes in PRIME cells (flow sorted CD45−/CD31−/PDPN+ cells) versus hematopoietic cells (flow sorted CD45+) and Log 2 fold change of Input cells (stained PBMC but not flow sorted) versus hematopoietic cells (flow sorted CD45+) as technical control for stress of flow sorting. These data show that single-cell marker genes of fibroblasts (SC-F1, SC-F2, SC-F3, SC-F4) but not B cells (SC-B1-4), macrophages (SC-M1-4), or T cells (SC-T1-6) are enriched in sorted PRIME cells. Fibroblast genes (as marked) were the only set of synovial cell marker genes enriched in PRIME cells.

FIG. 22 depicts Volcano plot of log 10 (−padj) vs Log 2 fold change of PRIME cells (flow sorted DAP−/CD45−/CD31−/PDPN+ cells) versus hematopoietic cells (flow sorted SAP−/CD45+). The results show that classic fibroblast genes are significantly increased in PRIME cells relative to hematopoetic cells.

FIG. 23 illustrates an exemplary process of refining the dataset to identify a panel of a smaller number of markers for prediction of an impending RA flare.

### DETAILED DESCRIPTION

In accordance with the present disclosure there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

### A. Terminology

The term “rheumatoid arthritis” or “RA” refers to a chronic disease, which is immune-mediated and inflammatory and is an autoimmune disorder, affecting the lining of joints that causes joint pain, stiffness, swelling and decreased movement of the joints and can eventually result in bone erosion and joint deformity. RA is a systemic autoimmune disease characterized by the simultaneous inflammation of the synovium of multiple joints.

An “RA flare” or “flare” refers to a surge in immune-mediated and/or inflammatory activity that is periodically experienced by a patient(s) with RA. During a flare, the level of fatigue and joint symptoms such as pain, swelling, and stiffness temporarily increase. Flares are periods of increased disease activity during which people's arthritis symptoms, which typically include joint pain, swelling, and stiffness, are more severe. An RA flare can involve an exacerbation of any symptom of the disease, but most commonly includes intense stiffness in the joints. People with RA report these common symptoms of flares: increased stiffness in joints, pain throughout the entire body, increased difficulty doing everyday tasks, swelling, such as causing shoes not to fit, intense fatigue, flu-like symptoms. The term “increased disease activity,” as used in this application, refers to an increase in disease activity score 28 (DAS28) or RAPID3. DAS28 is a composite score of RA related disease activity encompassing patient global assessment of RA activity, physician assessment of tenderness and swelling from 28 joints, and erythrocyte sedimentation rate (ESR) or C reactive protein (CRP). RAPID3 is a patient reported assessment of function, pain, and global health that correlates with DAS28 (Pincus, et al., Rheumatology (Oxford), 2008. 47(3):345-349; Pincus, et al., Arthritis Care Res (Hoboken), 2011. 63(8): p. 1142-9; Pincus, T., et al., J Rheumatol, 2011. 38(12): p. 2565-71; Pincus, et al., 2008. 35(11): p. 2136-47; Ward et al., J Rheumatol, 2019. 46(1): p. 27-30. Increased disease activity is further described in Example 1.

The term “antibody” describes an immunoglobulin whether natural or partly or wholly synthetically produced. The term also covers any polypeptide or protein having a binding domain which is, or is homologous to, an antibody binding domain. CDR grafted antibodies are also contemplated by this term. An “antibody” is any immunoglobulin, including antibodies and fragments thereof, that binds a specific epitope. The term encompasses polyclonal, monoclonal, and chimeric antibodies. The term “antibody(ies)” includes a wild type immunoglobulin (Ig) molecule, generally comprising four full length polypeptide chains, two heavy (H) chains and two light (L) chains, or an equivalent Ig homologue thereof (e.g., a camelid nanobody, which comprises only a heavy chain); including full length functional mutants, variants, or derivatives thereof, which retain the epitope binding features of an Ig molecule, and including dual specific, bispecific, multispecific, and dual variable domain antibodies; Immunoglobulin molecules can be of any class (e.g., IgG, IgE, IgM, IgD, IgA, and IgY), or subclass (e.g., IgG1, IgG2, IgG3, IgG4, IgA1, and IgA2). Also included within the meaning of the term “antibody” are any “antibody fragment”.

An “antibody fragment” means a molecule comprising at least one polypeptide chain that is not full length, including (i) a Fab fragment, which is a monovalent fragment consisting of the variable light (VL), variable heavy (VH), constant light (CL) and constant heavy 1 (CH1) domains; (ii) a F(ab′)2 fragment, which is a bivalent fragment comprising two Fab fragments linked by a disulfide bridge at the hinge region; (iii) a heavy chain portion of an Fab (Fd) fragment, which consists of the VH and CH1 domains; (iv) a variable fragment (Fv), which consists of the VL and VH domains of a single arm of an antibody, (v) a domain antibody (dAb) fragment, which comprises a single variable domain (Ward, E. S. et al., Nature 341, 544-546 (1989)); (vi) a camelid antibody; (vii) an isolated complementarity determining region (CDR); (viii) a Single Chain Fv Fragment wherein a VH domain and a VL domain are linked by a peptide linker which allows the two domains to associate to form an antigen binding site (Bird et al., Science, 242, 423-426, 1988; Huston et al., PNAS USA, 85, 5879-5883, 1988); (ix) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites (WO94/13804; P. Holliger et al. Proc. Natl. Acad. Sci. USA 90 6444-6448, (1993)); and (x) a linear antibody, which comprises a pair of tandem Fv segments (VH-CH1-VH-CH1) which, together with complementarity light chain polypeptides, form a pair of antigen binding regions; (xi) multivalent antibody fragments (scFv dimers, trimers and/or tetramers (Power and Hudson, J Immunol. Methods 242: 193-204 9 (2000)); (xii) a minibody, which is a bivalent molecule comprised of scFv fused to constant immunoglobulin domains, CH3 or CH4, wherein the constant CH3 or CH4 domains serve as dimerization domains (Olafsen T et al. (2004) Prot Eng Des Sel 17(4):315-323; Hollinger P and Hudson P J (2005) Nature Biotech 23(9):1126-1136); and (xiii) other non-full length portions of heavy and/or light chains, or mutants, variants, or derivatives thereof, alone or in any combination.

As antibodies can be modified in several ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included.

The term “adjuvant(s)” describes a substance, compound, agent or material useful for improving an immune response or immune cell or component stimulation and may in some instances be combined with any particular antigen in an immunological, pharmaceutical or vaccine composition. Adjuvants can be used to increase the amount of antibody and effector T cells produced and to reduce the quantity of antigen or immune stimulant or modulator and the frequency of injection. An adjuvant can serve as a tissue depot that slowly releases the antigen and as a lymphoid system activator that non-specifically enhances the immune response. In a preferred aspect an adjuvant is physiologically and/or pharmaceutically acceptable in a mammal, particularly a human.

The term “specific” may be used to refer to the situation in which one member of a specific binding pair will not show any significant binding to molecules other than its specific binding partner(s). The term is also applicable where e.g., an antigen binding domain is specific for a particular epitope which is carried by several antigens, in which case the specific binding member carrying the antigen binding domain will be able to bind to the various antigens carrying the epitope.

The term “comprise” generally used in the sense of include, that is to say permitting the presence of one or more features or components. The term “consist essentially of” refers to a product, such as a peptide sequence, of a defined number of residues which is not covalently attached to a larger product.

The term “oligonucleotide,” as used herein in referring to a probe of use the present disclosure, is defined as a molecule comprised of two or more ribonucleotides, preferably more than three. Its exact size will depend upon many factors which, in turn, depend upon the ultimate function and use of the oligonucleotide. The term “primer” as used herein refers to an oligonucleotide, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The term “agent” means any molecule, including polypeptides, antibodies, polynucleotides, chemical compounds and small molecules. In particular the term agent includes compounds such as test compounds or drug candidate compounds.

The term “assay” means any process used to measure a specific property of a compound. A “screening assay” means a process used to characterize or select compounds based upon their activity from a collection of compounds.

The term “protein” is used herein to mean protein, polypeptide, oligopeptide or peptide. The terms “protein marker”, “biomarker” or “protein marker of impending flare” are used herein to refer to proteins associated with or predictive or which precede specific diseases or conditions or symptoms, including proteins from or associated with aspects, cells or tissues affected by a disease or condition or symptom. In accordance with the present disclosure, the increase in marker expression relative to that detected or characteristic of a subject/s without overt organic RA disease or significant joint or pain symptoms or without an impending flare or a normal, healthy subject/s (control/controls) is positively correlated with, indicative of, or diagnostic for the impending presence or flare or flare-up of a disease or condition or symptoms thereof particularly an exacerbation of disease or symptoms, such as rheumatoid arthritis and particularly an RA flare, in a patient.

As used herein, the terms “increase” in marker expression or “differential expression” of a marker refer to a statistically significant increase or presence in a test sample (e.g., a sample from an RA patient) relative to a control sample when detected using the same assay under the same assay conditions. The term “increase” in expression or amounts with respect to a panel of markers refers to a statistically significant increase or presence of each marker in the panel. Typically assays for detecting expression of RNA markers are known, and the exemplary assays include RT-PCR, RNAseq, and the like. Typically assays for detecting the expression of protein markers are well known, and the exemplary assays include Western blot, ELISA, FACS, and the like. In some embodiments, an increase refers to that the amount of a marker detected in a blood sample from a patient is more than 10%, 20%, 30%, 40%, 50%, 60%, 70%, 80%, 90%, 200%, or 500% of the amount of the same marker in a control blood sample. In some embodiments, an increase includes an increase from 0 to any amount; for example, if a marker is not detectable in a control sample but is detected in a test sample in the same assay and under the same assay conditions, then the test sample is determined to have increased amount of the marker. The term statistically significant is used in the art to refer to the likelihood that a result or relationship is caused by something other than mere random chance. Statistical hypothesis testing is traditionally employed to determine if a result is statistically significant or not. Such testing provides a “p-value” representing the probability that random chance could explain the result. In general, a 5% or lower p-value is considered to be statistically significant.

The term “decrease” in marker expression or amount refer to a statistically significant decrease relative to controls when detected using the same assay and under the same assay conditions. A decrease in marker expression refers to that the amount of a marker detected in a blood sample from a patient is less than 90%, less than 80%, less than 70%, less than 60%, less than 50%, less than 40%, less than 30%, less than 20%, less than 10% of the amount of the same marker in a control blood sample. In some embodiments, a decrease also includes a decrease from any amount to 0 (the marker is absent in the test sample); for example, if a marker is detectable in a control sample but not detectable in a test sample in the same assay and under the same assay conditions, then test sample is determined to have decreased amount of the marker.

The term “a control blood sample” refers to a blood sample from a healthy individual. In some embodiments, the control blood sample can be a baseline sample from the same patient who is evaluated for the RA flare. In some embodiments, the control blood sample can be from a patient with unrelated illness, such as non-RA patients who have osteoarthritis.

A skilled practitioner would, moreover, appreciate that a relative increase or decrease in a particular protein (a protein marker) or a particular RNA (an RNA marker) in a sample alone may be weakly indicative or predictive of disease, but may not be diagnostic per se, if noted as a single determinant. If, however, a plurality of such single determinants is noted in a biological sample, the combined detection of several, even weakly indicative, determinants may serve to identify a strong combinatorial diagnostic indicator of impending disease or symptomatic disease aspects, such as impending RA flare. Furthermore, the single protein/determinant need not approach the threshold of weak diagnostic by itself but in combination with the detection of an increase of another protein or proteins or RNA or RNAs (other markers) may serve as a strong combinatorial diagnostic indication of an impending disease state. Accordingly, also encompassed herein are combinatorial diagnostic indicators or combinatorial markers that are associated with a particular disease or an impending disease and not observed in healthy subjects or patients with other diseases. For purpose of this disclosure, unless otherwise noted, a marker can refer to either the RNA transcribed from a gene or protein encoded by the same gene. For example, an AC3 marker (e.g., the KIAA1755 marker) can refer to either the RNA transcribed (e.g., the KIAA1755 RNA) from a gene in the AC3 panel or a protein (e.g., the KIAA1755 protein) encoded by the gene in the AC3 panel.

Accordingly, selected sets of one, two, three, several, at least 10, about 10, a dozen, 10-15, about 20, at least 20, 25, 30, about 30 and more, etc of the markers of this invention (up to the number equivalent to all of the markers, or all in a set of markers, including any intervening number, in whole number increments, e.g., 1, 2, 3, 4, 5, 6 . . . ) can be used as diagnostic indicators and predictors of an impending flare for methods and/or in kits described herein. In one embodiment, larger numbers of the markers identified herein are used in methods or kits of the present disclosure, since the accuracy of the method or kit may improve as the number of markers screened increases. With respect to aspects of the present disclosure pertaining to evaluating therapeutic efficacy, the methods and kits of the present disclosure include evaluating whether administration of a therapeutic composition causes a change, either a transient change or a long term change, in expression of one or more of the markers; in expression of two or more of the markers; in expression of three or more of the biomarkers; in expression of four or more of the biomarkers; in expression of five or more of the biomarkers, in expression of six or more of the biomarkers, etc.

As an example, a straightforward identification of a protein marker or an RNA marker is the presence of a protein or RNA associated with an impending disease or condition and not with other conditions that might be clinically confused with the disease under consideration. Variations to this scenario include the situation wherein a marker is present in an increased quantity compared to other conditions or controls. Although not a protein marker, an example is the presence of glucose in the blood in high quantities in diabetics compared to normal individuals who have glucose present but not in elevated quantities. A variation is where the functional marker is not just one protein but two or more in combination that can be quantitatively different, wherein the ensemble defines its marker potential.

In some embodiments, a marker of the present disclosure is a member of a biological pathway. As used herein, the term “precursor” or “successor” refers to molecules that precede or follow the marker in the biological pathway. Thus, once a marker is identified as a member of one or more biological pathways, the present disclosure include additional members of the biological pathway that come before (are upstream of or a precursor of) or follow (are downstream of) the marker. Such identification of biological pathways and their members is within the skill of one in the art.

Also encompassed herein is the analysis of markers identified and listed in the tables presented herein to identify metabolic pathways implicated in the pathogenesis, maintenance, and/or progression of a disease or an impending disease or system or flare. Such analyses may utilize a variety of software programs or approaches known and available to the skilled artisan. Multiple hits in a particular metabolic pathway underscore the potential importance of the pathway for the disease and direct therapeutic intervention toward appropriate modulation of same. Accordingly, the present methods encompass such analyses and the identification of metabolic pathways of potential significance in a particular disease or impending disease aspect or symptom. Knowing that, for example, activation of a metabolic pathway appears to be linked or associated with a particular disease or impending symptom presents the opportunity to test pharmaceutical modulators of the pathway (i.e., inhibitors) to determine if such modulators could be used as therapeutics for treatment of patients with the disease or impending disease or symptoms. This and these aspects are illustrated and described herein including in the examples.

In accordance with the present disclosure, the tables present information with which an ordinarily skilled practitioner can access the amino acid sequences of the proteins and RNAs identified herein as markers, as well as nucleic acid sequences encoding same. A stepwise protocol or means for identification of the sequences listed in the tables presented herein may include the artisan accessing one of the publicly available databases and entering the ensembl number or gene name or symbol to identify the sequence and relevant marker information. Such information may be used to design probes for detection of any of the proteins, genes, RNAs listed therein or to identify commercially available probes or antibodies therefore or thereof. Primers for detection of nucleic acid sequences encoding any of the proteins listed in the tables presented herein are also envisioned as are primers for PCR including RT PCR. Such primers may be used to detect RNA expression levels (including relative increases or decreases as compared to controls) of a marker relevant to the present disclosure. The design of primers for detecting expression levels of RNA (e.g., mRNA) of a marker or markers listed herein is a matter of routine practice with the nucleic acid sequence in hand as provided by publicly available websites such as those mentioned above. Such probes and primers are useful for the kits described herein.

The term “preventing” or “prevention” refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset. The term “prophylaxis” is related to and encompassed in the term “prevention” and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Therapeutically effective amount” means that amount of a drug, compound, antibody, or pharmaceutical agent that will elicit the biological or medical response of a subject that is being sought by a medical doctor or other clinician. In particular, with regard to gram-positive bacterial infections and growth of gram-positive bacteria, the term “effective amount” is intended to include an effective amount of a compound or agent that will bring about a biologically meaningful decrease in the amount of or extent of disease or flare free time period and or increase in length of a subject's survival or period disease-free or in remission or free of flare(s). The phrase “therapeutically effective amount” is used herein to mean an amount sufficient to prevent, and preferably reduce by at least about 30 percent, more preferably by at least 50 percent, most preferably by at least 90 percent, a clinically significant change, or enhanced survival or disease-free period by at least about 30 percent, more preferably by at least 50 percent, most preferably by at least 90 percent.

The term “treating” or “treatment” of any disease, condition, or infection refers, in one embodiment, to ameliorating the disease or infection (i.e., arresting the disease or growth of the infectious agent or bacteria or reducing the manifestation, extent or severity of at least one of the clinical symptoms thereof). In another embodiment “treating” or “treatment” refers to ameliorating at least one physical parameter, which may not be discernible by the subject. In another embodiment, “treating” or “treatment” refers to modulating the disease or infection, either physically, (e.g., stabilization of a discernible symptom), physiologically, (e.g., stabilization of a physical parameter), or both. In a further embodiment, “treating” or “treatment” relates to slowing the progression of a disease or reducing an infection. In some embodiments, “treating” a patient having or being diagnosed with an impending RA flare refers to treating the patient to prevent an impending flare or ameliorate symptoms related to the flare, for example, resulting in the patient experiencing a reduced flare, fewer pathologies and/or symptoms associated with a flare, or resulting in a flare and its associated disease exacerbations being reduced, limited in duration, or avoided.

The phrase “pharmaceutically acceptable” refers to molecular entities and compositions that are physiologically tolerable and do not typically produce an allergic or similar untoward reaction, such as gastric upset, dizziness and the like, when administered to a human.

As used herein, “pg” means picogram, “ng” means nanogram, “ug” or “μg” mean microgram, “mg” means milligram, “ul” or “μl” mean microliter, “ml” means milliliter, “l” means liter.

This application incorporates by reference of the entire content of the following publication: Orange et al., N. Engl. J. Med. 383: 218-28 (2020), available at DOI: 10.1056/NEJMoa2004114.

### B. Detailed Disclosure

The present disclosure relates to and provides previously unidentified and unrecognized markers, particularly RNA markers and protein markers, which are indicators and antecedents of an impending rheumatoid arthritis (RA) flare. The markers are differentially expressed or preferentially expressed prior to an RA flare in an RA patient and the expression profile of which can be used to predict an impending flare and can be utilized to implement and prescribe treatment and therapy to a patient. In some embodiments, an antecedent marker can be an RNA marker (e.g., an AC3 RNA marker) or a protein marker (a AC3 protein marker) so long as they are differentially expressed or preferentially expressed before an RA flare.

### RA

Arthritis is a disease that may cause damage to the healthy cartilage of joints, leading to degenerative changes, loss of function and joint instability. Inflammatory arthritis describes conditions characterized by pain, swelling, tenderness and warmth in the joints, as well as morning stiffness that lasts for more than an hour. An increase of cytokines leads to degradation of articular cartilage and a decrease of growth factors which induce chondrogenesis in inflammatory arthritis. The most common inflammatory arthritis associated disorders rheumatoid arthritis (RA), psoriatic arthritis (PsA), systemic lupus erythematosus (SLE, lupus), ankylosing spondylitis (AS), and gouty arthritis (gout). Arthritis damages the cartilage within joints, resulting in degenerative changes, including loss of function and joint instability. Ankylosing spondylitis (AS) is a chronic inflammatory condition affecting the spine and bone-to-tendon attachment area within the sacroiliac joint leading to back pain and progressive spinal stiffness. Rheumatoid arthritis is a chronic, systemic autoimmune disease characterized by the simultaneous inflammation of the synovium of multiple joints, leading to joint damage (e.g., destruction, deformation and disability). Gout is a chronic inflammatory disease that causes an alteration of joints resulting in severe pain and is associated with an accumulation of uric acid within the body resulting from dysregulated purine metabolism, causing recurrent paroxysmal inflammation in the joints. Allopurinol and febuxostat are the primary treatment options for individuals with gout.

Various disease-modifying agents for treating or modifying RA, including for management and alleviation of RA flares, are known and in use clinicically. Nonsteroidal anti-inflammatory drugs (NSAIDs) or disease-modifying antirheumatic drug (DMARDs) have been used for the treatment of these inflammatory diseases, particularly RA. More recently, biologic DMARDs have been introduced with excellent results. NSAIDs include non-prescription drugs acetylsalicylate (aspirin), ibuprofen (Advil, Motrin IB) and naproxen sodium (Aleve, Naprosyn) and prescription NSAIDs such as etodolac (Lodine) and diclofenac (Voltaren). Steroids are anti-inflammatory or immunosuppressants agents and are prescribed for more severe RA or when RA symptoms flare to ease joint pain and stiffness. Examples include glucocotricosteroids or corticosteroids such as prednisone, cortisone and methylprednisolone. DMARDs are prescribed and utilized to slow the progression of RA and save joints and other tissues from permanent damage. Common conventional DMARDs include methotrexate (Trexall, Otrexup), leflunomide (Arava), hydroxychloroquine (Plaquenil) and sulfasalazine (Azulfidine). DMARDs curb the overactive immune system in RA but aren't selective in their targets. Biologics—genetically engineered proteins which target a specific aspect or part of the immune system and act as immunosuppressants—are an increasingly important component in treatment of RA and are commonly denoted biologic DMARDs or bDMARDs. Biologics include abatacept (Orencia), adalimumab (Humira), anakinra (Kineret), baricitinib (Olumiant), certolizumab (Cimzia), etanercept (Enbrel), golimumab (Simponi), infliximab (Remicade), rituximab (Rituxan), sarilumab (Kevzara), tocilizumab (Actemra) and tofacitinib (Xeljanz). Adalimumab, etanercept, infliximab, golimumab and certolizumab target tumor necrosis factor (TNF). Rituximab is effective against B cells. Anakinra blocks the action of interleukin-1 (IL-1), a master cytokine. Abatacept targets T cells. Biologic DMARDs are usually most effective when paired with a nonbiologic DMARD, such as methotrexate. New DMARD drugs which are specific in their targets but are not biologics include oral small molecule Janus kinase (JAK) inhibitors such as tofacitinib (Xeljanz and Xeljanz XR), baricitinib (Olumiant), and upadacitinib (Rinvoq).

The American College of Rheumatology (ACR) has recommendations for RA patient treatment given various disease parameters (e.g., Singh J et al. (2016) Arthritis Rheumatolo 68:1-26). Disease activity scales are utilized in managing RA patients and choosing appropriate treatment modalities, including the routine assessment of patient index data 3 (RAPID3) and the disease activity score 28 (DAS28) (Fransen, J et al. (2003) Arthritis Rheum 49 Suppl:S214-24), which incorporates tenderness and swelling from 28 joints, erythrocyte sedimentation rate (ESR) and patient global assessment of disease activity, both of which have been utilized in studies described herein. Additional assessment instruments include Patient Activity Sale (PAS) or PASII (Wolfe, F et al. (2005) J Rheumatol 32:2410-5), Clinical Disease Activity Index (CDAI) (Aletaha, D et al. (2005) Arthritis Res Ther 7: R796-806) and Simplified Disease Activit Index (SDAI) (Smolen, J S et al. (2003) Rheumatology 42:244-57). Each of these scales and assessments are applicable in treatment once a patient has symptomatic aspects or indicators of a flare or of disease. These scales cannot predict a flare, they are indicators of a flare or of exacerbation of disease.

RA patients are not able to predict a flare and are therefore subject to unpredictable exacerbations of disease and disease-associated symptoms and continual, progressive joint damage. The availability of dependable markers of impending flare(s), which can be readily and reliably assessed, particularly without significant clinical intervention, would significantly impact the treatment and management of RA patients and reduce the impact and long-term effects of the disease.

### AC2 Markers

Toward that end, the present disclosure provides a first set of markers that are expressed two weeks or about two weeks prior to an RA flare. These markers are referred to as the AC2 markers. The timing before flare can have a margin of error of about a week or 7 days. Referring to the studies provided herein, patients symptoms were assessed using a questionnaire that asked about how they were doing over the past week, therefore there is a possible seven-day margin of error in timing. For instance, given the questionnaire, the researchers could not necessarily discriminate between symptoms that started that day (or on day 1) vs 6 days earlier, or about a week or up to 7 days earlier. Therefore, the timing of the first set of markers is about two weeks, with a margin of error up to an additional 7 days, therefore up to 3 weeks or a week up to three weeks or 7-21 days. A first set of markers can be identified and characterized in a patient sample, particularly a blood sample, including a fingerstick sample of blood, two weeks or about two weeks, about 14 days, approximately 14 days, more than one week, more than 12 days, more than 10 days, about 10-14 days, about 12-14 days prior to an RA flare, with a margin of error in each instance of up to about a week or 7 days, therefore, with a margin of error up to three weeks, at least a week, about two or three weeks, two or three weeks, about 7-21 days, up to 21 days, at least 7-10 days, about two to three weeks prior to an RA flare. These actecedent markers, particularly denoted AC2 markers, are provided in Table 7. As shown in Example 2 and Table 7, the RNA analysis of fingerstick blood samples from RA patients indicate that all AC2 RNA markers listed in Table 7 increased the week prior to flare and was then decreased for the duration of the flare.

### AC3 Markers

Another and second set of markers are provided that are expressed one week or about one week, or about 7 days, approximately 7 days, prior to an RA flare. These markers are also referred to as the AC3 markers. The timing before flare can have a margin of error of about a week or 7 days. Referring to the studies provided herein, patients symptoms were assessed using a questionnaire that asked about how they were doing over the past week, therefore there is a possible seven-day margin of error in timing. For instance, given the questionnaire, the researchers could not necessarily discriminate between symptoms that started that day (or on day 1) vs 6 days earlier, or about a week or up to 7 days earlier. The second set of markers can be identified and characterized in a patient sample, particularly a blood sample, including a fingerstick sample of blood, about one week, or about 7 days, approximately 7 days, about 5-7 days prior to an RA flare, with a margin of error in each instance of up to about a week or 7 days, therefore, with a margin of error up to two weeks, up to 14 days, 0-14 days, about one to two weeks, at least a week, about a week to 10 days, 7-14 days, 5-14 days prior to a flare. These antecedent markers, particularly denoted AC3 markers, are provided in Table 8. The set of AC2 markers or the first set of markers are expressed further out from a flare and more than a week before flare, up to three weeks prior to an RA flaree, while the set of AC3 markers or the second set of markers are expressed thereafter or closer to a flare and about a week or up to two weeks prior to a flare. See Example 2.

In an aspect, the AC3 markers, or one or more AC3 marker, or AC3 markers which are sublining fibroblast genes, are decreased during flare or after the commencement of a flare or once a patient experiences physical indicators or symptoms of a flare. Physical indicators or symptoms of a flare may be selected from stiffness in joints, pain throughout the body, increased difficulty doing everyday tasks, swelling, fatigue and flu-like symptoms. In an aspect, synovial cell marker genes or proteins among the AC3 markers and Table 8 are selected. A flare(s) may be evaluated by recognition of the symptoms in a patient and/or utilizing any recognized disease activity scales, including as described and provided herein.

RNA markers and protein markers of impending flare which are particularly selected for determining and predicting impending RA flares are provided in Table 9. The markers COL1A2, COL5A1, COL16A1, COL14A1, COL4A2, PXDN, ST5, DCLK1, SCARA5, EGFR, EGR1 and ZFHX4. RNAs or their encoded proteins selected from COL1A2, COL5A1, COL16A1, COL14A1, COL4A2, PXDN, ST5, DCLK1, SCARA5, EGFR, EGR1 and ZFHX4 are expressed one week or about one week, or about 7 days, approximately 7 days, about 5-7 days, at least 5 days prior to an RA flare. Markers selected from COL1A2, COL5A1, COL16A1, COL14A1, COL4A2, PXDN, ST5, DCLK1, SCARA5, EGFR, EGR1 and ZFHX4. RNAs or their encoded proteins selected from COL1A2, COL5A1, COL16A1, COL14A1, COL4A2, PXDN, ST5, DCLK1, SCARA5, EGFR, EGR1 and ZFHX4 are expressed one week or about one week, or about 7 days, approximately 7 days, about 5-7 days, at least 5 days prior to an RA flare. In particular, the markers are differentially expressed in a patient prior to a flare. Markers selected from COL1A2, COL5A1, COL16A1, COL14A1, COL4A2, PXDN, ST5, DCLK1, SCARA5, EGFR, EGR1 and ZFHX4. RNAs or their encoded proteins selected from COL1A2, COL5A1, COL16A1, COL14A1, COL4A2, PXDN, ST5, DCLK1, SCARA5, EGFR, EGR1 and ZFHX4 are differentially expressed, their expression is increased or higher relative to other markers or proteins, or their expression is significantly increased relative to expression in a normal sample or a sample from an individual that does not have RA or any other recognized inflammatory or autoimmune disease, one week or about one week, or about 7 days, approximately 7 days, about 5-7 days, at least 5 days prior to an RA flare. As noted above, the margin of error in timing may be up to a week or 7 days. Therefore expression of these markers selected from COL1A2, COL5A1, COL16A1, COL14A1, COL4A2, PXDN, ST5, DCLK1, SCARA5, EGFR, EGR1 and ZFHX4. RNAs or their encoded proteins selected from COL1A2, COL5A1, COL16A1, COL14A1, COL4A2, PXDN, ST5, DCLK1, SCARA5, EGFR, EGR1 and ZFHX4 maybe increased one top two weeks or 0-14 days, or about a week or two prior to a flare. Expression of RNAs or their encoded proteins selected from COL1A2, COL5A1, COL16A1, COL14A1, COL4A2, PXDN, ST5, DCLK1, SCARA5, EGFR, EGR1 and ZFHX4 are decreased in peripheral blood during an RA flare. Expression of RNAs or their encoded proteins selected from COL1A2, COL5A1, COL16A1, COL14A1, COL4A2, PXDN, ST5, DCLK1, SCARA5, EGFR, EGR1 and ZFHX4 are decreased in peripheral blood during an RA flare in comparison to their expression prior to a flare, particularly their expression about a week, or up to two weeks, prior to a flare.

RNA markers and protein markers of impending flare which are particularly selected for determining and predicting impending RA flares are provided in Table 12. The markers COL14A1, DCLK1, FNDC1, COL16A1, COL1A2, KIAA1755, PXDN, and COL5A1. RNAs or their encoded proteins selected from COL14A1, DCLK1, FNDC1, COL16A1, COL1A2, KIAA1755, PXDN, and COL5A1 are expressed one week or about one week, or about 7 days, approximately 7 days, about 5-7 days, at least 5 days prior to an RA flare. Markers selected from COL14A1, DCLK1, FNDC1, COL16A1, COL1A2, KIAA1755, PXDN, and COL5A1. RNAs or their encoded proteins selected from COL14A1, DCLK1, FNDC1, COL16A1, COL1A2, KIAA1755, PXDN, and COL5A1 are expressed one week or about one week, or about 7 days, approximately 7 days, about 5-7 days, at least 5 days prior to an RA flare. In particular, the markers are differentially expressed in a patient prior to a flare. Markers selected from COL14A1, DCLK1, FNDC1, COL16A1, COL1A2, KIAA1755, PXDN, and COL5A1. RNAs or their encoded proteins selected from COL14A1, DCLK1, FNDC1, COL16A1, COL1A2, KIAA1755, PXDN, and COL5A1 are differentially expressed, their expression is increased or higher relative to other markers or proteins, or their expression is significantly increased relative to expression in a normal sample or a sample from an individual that does not have RA or any other recognized inflammatory or autoimmune disease, one week or about one week, or about 7 days, approximately 7 days, about 5-7 days, at least 5 days prior to an RA flare. As noted above, the margin of error in timing may be up to a week or 7 days. Therefore, expression of these markers selected from COL14A1, DCLK1, FNDC1, COL16A1, COL1A2, KIAA1755, PXDN, and COL5AL. RNAs or their encoded proteins selected from COL14A1, DCLK1, FNDC1, COL16A1, COL1A2, KIAA1755, PXDN, and COL5A1 maybe increased one top two weeks or 0-14 days, or about a week or two prior to a flare. Expression of RNAs or their encoded proteins selected from COL14A1, DCLK1, FNDC1, COL16A1, COL1A2, KIAA1755, PXDN, and COL5A1 are decreased in peripheral blood during an RA flare. Expression of RNAs or their encoded proteins selected from COL14A1, DCLK1, FNDC1, COL16A1, COL1A2, KIAA1755, PXDN, and COL5A1 are decreased in peripheral blood during an RA flare in comparison to their expression prior to a flare, particularly their expression about a week, or up to two weeks, prior to a flare.

RNA markers and transcripts or protein markers common to synovial sublining fibroblasts which are markers of impending flare and are capable of predicting or determining an impending flare or increased disease activity as set out in Table 5. The markers comprise or consist of COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6. The markers are COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6. The markers are selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6. RNAs or their encoded proteins selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 are expressed one week or about one week, or about 7 days, approximately 7 days, about 5-7 days, at least 5 days, prior to an RA flare. As noted above, the margin of error in timing may be up to a week or 7 days. Therefore, expression of these markers of impending flare may be found or evident a week or up to two weeks, at least about a week, about 0-14 days, up to two weeks, 5-14 days prior to a flare. Expression of RNAs or their encoded proteins selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 are, decreased in peripheral blood during an RA flare.

### Refined AC3 Markers

In some embodiments, the method of predicting an impending RA flare includes detecting in the blood sample increased amounts of a panel of AC3 markers, wherein the panel of antecedent AC3 markers comprises or consists of one or more markers listed in Table 10, for example, at least 5, at least 10, at least 50, at least 100 markers. In some embodiments, the panel of antecedent AC3 markers comprise or consist of 2-283 markers, e.g., 8-200, 8-180, or 10-150 markers of the markers listed in Table 10, including all markers listed in Table 12. In some embodiments, the panel comprise or consist of 65-283 markers of the markers listed in Table 10, including some or all markers listed in Table 11. In some embodiments, the panel comprise or consist of all markers listed in Table 10. As shown in Example 3, genes listed in Table 10 are expressed in high levels (top quartile) in blood samples from RA patients and thus can be used for prediction of an impending RA with high confidence. The detection of increased amounts of these AC3 markers indicates an impending RA flare in about a week or up to two weeks.

In some embodiments, the panel comprise or consist of one or more markers listed in Table 11, for example, at least 5, at least 10, at least 20, at least 30 markers of the markers listed in Table 11. In some embodiments, the panel comprises or consists of 2-65 markers, e.g., 5-65 markers, 10-60 markers, or 20-55 markers, including some or all markers listed in Table 12. In some embodiments, the panel comprises or consists of all markers listed in Table 11. In some embodiments, the panel comprises or consists of all markers listed in Table 12. As shown in Example 3, markers listed in Table 11 overlap with published synovial marker genes in a published dataset (accession #SDY998). This overlap is useful because it increases the specificity of the gene list by enriching for genes that are not typically expressed in circulating white blood cells, thus more likely be indicators for disease conditions. The detection of significantly increased amounts of these AC3 markers (for example, having a fold change with a P value less than 0.05) indicates an impending RA flare in about a week or up to two weeks.

In some embodiments, the panel comprises or consist of one or more markers listed in Table 12, for example, at least 5, at least 6, at least 7 markers listed in Table 11. In some embodiments, the panel comprises or consists of 2-8 markers, e.g., 4-8 markers, 5-8 markers, 6-8 markers, or 7-8 markers of those listed in Table 12. In some embodiments, the panel comprises or consists of all markers listed in Table 12, i.e., COL14A1, DCLK1, FNDC1, COL16A1, COL1A2, KIAA1755, PXDN, and COL5AL. As shown in Example 3, genes listed in Table 12 were enriched for synovial sublining genes relative to fibroblast genes. The detection of increased amounts of these AC3 markers indicates an impending RA flare in about a week or up to two weeks.

In some embodiments, a panel of at least 20 of the AC2 or AC3 markers described above may be evaluated. In an aspect, a panel of at least 10 of the AC2 or AC3 markers are evaluated. At least 4, at least 5, at least 6, at least 7, at least 8, at least 9, at least 10, at least 11, at least 12, at least 13, at least 14, least 15, at least 16, at least 17, at least 18, at least 19, at least 20, at least 25, at least 30, at least 35, at least 40, at least 45, at least 50 of the AC2 or AC3 markers are evaluated to determine their amounts in a blood sample from the patient. A panel of at least 20 of the AC2 and at least 20 of the AC3 markers may be evaluated. In an aspect, a panel of at least 10 of the AC2 and the AC3 markers are evaluated. At least 4, at least 5, at least 6, at least 7, at least 8, at least 9, at least 10, at least 11, at least 12, at least 13, at least 14, least 15, at least 16, at least 17, at least 18, at least 19, at least 20, at least 25, at least 30, at least 35, at least 40, at least 45, at least 50 of the AC2 and the AC3 markers are evaluated.

### Cell Markers

In accordance with the present disclosure herein, the recognition of unique markers in peripheral blood has led to the identification and characterization of a distinctive and specific cell or cell type circulating in the blood of a patient or individual prior to a flare. This unique and specific blood circulating cell is a novel indicator of an impending RA flare. The present disclosure includes a unique blood circulating cell, denoted a Pre-Inflammatory mesenchymal (PRIME) cell, which has been identified and characterized as circulating in peripheral blood in a patient, particularly an RA patient, particularly a human, prior to an RA flare. The presence of this cell in peripheral blood indicates that an RA flare will occur or become evident by way of one or more patient symptom(s). The cell can be identified in patient peripheral blood about one week, one week, about 7 days, about 5-8 days, about 5-7 days, 5-8 days, 5-7 days, about 4-7 days, 4-7 days, about 3-7 days, 3-7 days prior to an RA flare. The cell can be identified in patient peripheral blood about one week, one week, about 7 days, about 5-8 days, about 5-7 days, 5-8 days, 5-7 days, about 4-7 days, 4-7 days, about 3-7 days, 3-7 days prior to inflammation of one or more joints or pain in one or more joints in a patient. As noted above, the margin of error in timing may be up to a week or about 7 days. Therefore, the cell can be identified in patient peripheral blood about one week to about 2 weeks, about 7-14 days, up to 14 days, 0-14 days, about 3-14 days, about 5-14 days prior to an RA flare. In an embodiment, PRIME cell(s) can be identified and characterized as a CD45−CD31− PDPN+ cell, particularly as a CD45−CD31−PDPN+ cell in peripheral blood. In another embodiment, PRIME cell(s) can be identified and characterized as a CD45−CD31−PDPN+IL-17RD+ cell, particularly as a CD45−CD31−PDPN+IL-17RD+ cell in peripheral blood.

In an embodiment, identifying and characterizing the presence of CD45−CD31−PDPN+ cells or CD45−CD31−PDPN+IL-17RD+ cells in peripheral blood provides a diagnostic which is predictive of an impending flare in an RA patient. In an embodiment, identifying and characterizing the presence of CD45−CD31−PDPN+ cells or CD45−CD31−PDPN+IL-17RD+ cells in peripheral blood provides a diagnostic which is predictive of an impending flare, or of stiffness in joints, pain throughout the body, increased difficulty doing everyday tasks, swelling, fatigue and/or flu-like symptoms in a patient, including an RA patient, or a patient which is suspected of having RA or an arthritic or inflammatory disease.

The present disclosure thus provides a method for monitoring and predicting a rheumatoid arthritis (RA) flare in a patient comprising:
(a) optionally isolating a blood sample from said patient;
(b) evaluating the blood sample for expression or quantitatively increased amounts of one or more sets of antecedent RNA markers, protein markers or cell markers selected from:
(i) AC2 markers or proteins as provided in Table 7;
(ii) AC3 markers or proteins as provided in Table 8;
(iii) markers or proteins selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 as set out in Table 5;
(iv) markers or proteins selected from COL1A2, COL5A1, COL16A1, COL14A1, COL4A2, PXDN, ST5, DCLK1, SCARA5, EGFR, EGR1 and ZFHX4 as set out in Table 9;
(v) markers or proteins selected from COL14A1, DCLK1, FNDC1, COL16A1, COL1A2, KIAA1755, PXDN, and COL5A1;
(vi) cell markers CD45− CD31−PDPN+;
(c) wherein the expression or quantitatively increased amounts of the RNA markers or proteins or the presence of the cell markers predicts an impending RA flare.

The expression or quantitatively increased amounts of the AC2 RNA markers or proteins predicts an RA flare in about 2 weeks or about 12-14 days, with a margin of error of up to about a week or 7 days, thus in about 7-21 days. Differential expression of AC2 markers has been identified and characterized in RA patients approximately two weeks prior to the presence of symptoms indicative of an RA flare in the patients. The expression or quantitatively increased amounts of the AC3 RNA markers or proteins predicts an RA flare in about 1 week or about 5-7 days, with a margin of error of about a week or 7 days, thus in about a week or up to 2 weeks, or up to 14 days. Differential expression of AC3 markers has been identified and characterized in RA patients approximately one week or about 5-7 days prior to the presence of symptoms indicative of an RA flare in the patients. The period of time prior to recognition of flare symptoms may vary by a day, a few days or several days from either a week before or two weeks before. The variation may be as a result of the timing of blood collection or sample collection for evaluation of the marker(s). The variation may be as a result of the sensitivity of the patient to symptoms of an RA flare or the ability of a patient to identify or recognize the symptoms or any clinical parameter of a flare. In as much as physical indicators or symptoms of a flare may be selected from stiffness in joints, pain throughout the body, increased difficulty doing everyday tasks, swelling, fatigue and flu-like symptoms, these may be recognized immediately or in the short term or may be recognized after a day or two or several days of symptoms in and by a patient.

Any applicable and sufficient number of markers may be evaluated in a patient to determine or predict an impending flare. Thus, the markers should be sufficient to reliably predict an impending flare. One skilled in the art will be able to utilize the data herein and available to provide a set of markers necessary or sufficient to predict a flare. In particular and for example, markers which are associated with certain pathways or responses may be selected. Pathways involved in myeloid, neutrophil, Fc receptor signaling and platelet activation may be selected. Genes or markers associated with developmental pathways for naïve B cells and leukocytes may be selected from among the AC2 genes. Naïve B cell genes may be selected from among AC2 genes. Pathways related to extracellular matrix, collagen and connective tissue development may be selected, and may be particularly selected from among the AC3 genes. The present disclosure describes that AC3 was enriched for pathways not typical of blood samples, including cartilage morphogenesis, endochondral bone growth, and extracellular matrix organization. Genes for these pathways or associated with these may be selected from the AC3 genes as markers for predicting RA flares. AC3 is described as enriched with sublining fibroblast genes, in a particular aspect the sublining fibroblast genes CD34+, HLA-DR+, and DKK3+. AC3 is described as enriched with sublining fibroblast genes, in a particular aspect the sublining fibroblast genes CD45−CD34+, CD45− HLA-DR+, and CD45−DKK3+. In an aspect, these may be particularly selected from or included in the markers selected from the AC3 genes. In an embodiment, sublining fibroblast markers selected from the AC3 markers or proteins are selected and evaluated. In an embodiment, AC3 RNA markers or protein markers expressed by CD34+, HLADR+ and DKK3+ cells are evaluated. In an embodiment, AC3 markers or proteins expressed by CD45−CD34+, CD45−HLADR+ and CD45−DKK3+ cells are evaluated. The method includes wherein the cell marker IL-17RD is also evaluated. In an embodiment, AC3 RNA markers or protein markers expressed by PRIME cells, CD45−CD31−PDPN+ cells, or CD45−CD31− PDPN+IL-17RD+ cells are evaluated.

Notably, many of the relevant and most particular markers provided herein are unusual in peripheral blood and/or are not necessarily or particularly associated with inflammation or an inflammatory condition per se. This facilitates their specificity, relevance and significance in particularly or specifically predicting or implying an impending RA flare, and/or exacerbation of joint symptoms. Some prior marker studies have identified inflammatory genes, such as inflammation gene expression panels, wherein the genes are associated with RA or inflammatory type conditions of RA, such as described in U.S. Pat. No. 7,935,482. These inflammatory genes provide a distinct profile and profiling from those provided herein, are particularly skewed and relevant for inflammation, and are not predictive of an impending flare.

### Sample Collection

In some embodiments, a method of predicting or preventing an impending RA disclosed herein uses a small volume collected via fingerstick (e.g., using a deep lancet). In some embodiments, the sample volume is less than 500 μL, less than 300 μL, less than 250 μL, less than 200 μL, e.g., about 150 uL. In some embodiments, the sample volume is about 100-300 μL, 50-300 μL, 50-250 μL, or 50-200 μL. In some embodiments, the sample volume is less than 100 μL, less than 50 μL, about 10-50 μL, about 8-15 μL, about 10-20 μL, or about 10 μL. In some embodiments, the fingerstick sample may comprise blood droplets directly from a fingerstick, e.g., using a 21 gauge×1.8 mm deep lancet. In some embodiments, the blood droplets are collected via a capillary tube. In some embodiments, the blood droplets are collected in a microtainer tube, e.g., BD Microtainer® blood collection tubes, available from Becton, Dickinson and Company, Franklin Lakes, NJ. As compared to conventional methods of collecting blood via, e.g., by venipuncture, collecting blood by fingerstick can be performed by patients at home with ease. The volume is small and does not cause substantial discomfort to patients. After the blood sample is drawn, it can be stored and mailed to a test center to be analyzed. Methods of collecting blood samples are well known and also are described in Example 1. As shown in Example 1, the results of RNAseq analyses of these fingerstick blood samples demonstrate that the data correlate well the results obtained from conventional, gold standard clinical measurement of blood counts. Thus, in some embodiments, the method comprises collecting a blood sample from a patient by fingerstick, and the sample is then analyzed to detect whether there are increased amounts of the antecedent RA markers for determining if the patient will have an impending RA flare or increased disease activity as disclosed herein.

### Selecting Patients

In some embodiments, a method of predicting or preventing an impending RA disclosed herein comprises selecting a patient who has been determined to have increased amounts of a panel of antecedent RA markers as disclosed herein, and treating the patient with a therapeutically effective amount of one or more disease-modifying agent. In some embodiments, the panel of antecedent markers comprises or consists of one or more markers listed in Table 10, for example, at least 5, at least 10, at least 50, at least 100 markers. In some embodiments, the panel comprise or consist of 2-283 markers, e.g., 8-200, 8-180, or 10-150 markers of the markers listed in Table 10, including all markers listed in Table 12. In some embodiments, the panel comprise or consist of 65-283 of the markers listed in Table 10, including some or all markers listed in Table 11. In some embodiments, the panel comprise or consist of all markers listed in Table 10. In some embodiments, the panel comprises or consists of one or more markers listed in Table 11, for example, at least 5, at least 10, at least 20, at least 30 markers of the markers listed in Table 11. In some embodiments, the panel comprise or consists of 2-65 marker, e.g., 5-65 markers, 10-60 markers, or 20-55 markers, including some or all markers listed in Table 12. In some embodiments, the panel comprise or consist of all markers listed in Table 11. In some embodiments, the panel comprise or consist of one or more markers listed in Table 12, for example, at least 5, at least 6, at least 7 markers listed in Table 11. In some embodiments, the panel comprise or consists of 2-8 markers, e.g., 4-8 markers, 5-8 markers, 6-8 markers, or 7-8 markers of those listed in Table 12. In some embodiments, the panel comprise or consists of at least 2, at least 3, at least 4, at least 5, at least 6 at least 7 or all markers of those listed in Table 12. In some embodiments, the panel comprises or consists of all markers listed in Table 12, i.e., COL14A1, DCLK1, FNDC1, COL16A1, COL1A2, KIAA1755, PXDN, and COL5A1.

### Methods of Determining the Amounts of the Antecedent RA Markers

Polypeptide or protein markers and RNA(s) or RNA markers may be isolated or evaluated by any suitable method known in the art. Proteins or RNAs can be purified or assayed by standard methods known in the art, such as via immunoassay, ELISA, nucleic acid probes, primers, oligonucleotides, antibody affinity methods, RNA sequencing, and the like. In one embodiment, polypeptide and metabolite markers may be isolated from a biological sample using standard techniques known in the art, for example, affinity purification using substrate-bound antibodies that specifically bind to the marker. As described herein, immunoaffinity depletion of abundant RNA(s) or proteins (with masking potential) enhances coverage and detection of low abundance proteins or RNA(s).

Antibodies immunospecific for any one of the markers provided herein may be known and available to the public, and they may be accessed via the scientific community or purchased from a commercial vendor. Readily searchable databases or web browsers may be utilized, for example, for identifying potential suppliers for such antibodies.

In some embodiments, the markers are RNA markers, and increased amounts of the RNA markers in the blood sample can be detected using methods well known, for example, RT-PCR or RNAseq. In some embodiments, RNA is extracted from the blood sample and the isolated RNA is sequenced using any suitable methods and kits. In some embodiments, kits such as Illumina TruSeq or Kapa Hyper Prep Kits are used for sequencing. In some embodiments, the isolated RNA is first converted to cDNA before sequencing. In some embodiments, the cDNAs are amplified and amplification products are sequenced. The expression or amount of the RNA marker is quantified by counting the number of reads that mapped to the marker.

In some embodiments, the markers are protein markers and increased amounts of the protein markers in the blood sample can be detected using methods well known in the art, for example, ELISA, Westernblots, and the like. The detection of increased amounts of the markers (RNA and/or protein markers) in a panel disclosed above, for example, a panel comprising one or more or all markers in Table 12, indicates an impending RA flare.

Treating a Patient with an Impending RA

The present disclosure particularly relates to predicting an impending RA flare in a patient and treating the patient for the impending flare so that the patient experiences a reduced flare, fewer pathologies and/or symptoms associated with a flare, or such that a flare and its associated disease exacerbations are reduced, limited in duration, or avoided. This a method is provided herein for predicting an impending flare and treating a patient diagnosed with an impending flare, so that prophylaxis may be achieved. This serves to significantly reduce the disease and the associated difficulties for an RA patient and provides a clinically more stable RA scenario.

In some embodiments, the method for predicting and/or treating an impending RA flare or increased disease activity in a patient comprises: a) contacting a blood sample from the patient with reagents specific for a panel of AC3 markers, wherein the panel comprise one or more, or all AC3 markers listed in Table 10, Table 11, or Table 12, b) detecting amounts of the markers of the panel in the blood sample, c) comparing the amounts of the markers in the panel to the amounts of the markers in a control blood sample, where increased amounts indicates an impending RA flare in a patient, and optionally d) administering a therapeutically effective amount of one or more disease-modifying agent for treating RA if the amounts of the markers of the panel in the blood sample is increased relative to the amounts of the markers in the control blood sample, thereby treating the impending flare in the patient. In some embodiments, the markers are RNA markers and detecting the amounts of the markers comprises reversely transcribing the RNA markers into cDNAs, and amplifying the cDNAs using primers to produce amplification products. In some embodiments, the markers are RNA markers listed in Table 12 and the cDNA reversely transcribed from these markers are amplified before sequencing. In some embodiments, cDNAs of these markers are amplified using the primer sets that are suitable for amplifying these markers, for example those listed in Table 13.

In some embodiments, methods are provided for predicting an impending RA flare and/or treating a patient to prevent an impending flare in a patient, including comprising:
a) isolating a blood sample from the patient;
b) contacting the blood sample with reagents specific for markers selected from a panel of RNA or protein markers to assess expression of the RNA or protein markers, wherein the panel of RNA or protein markers is selected from:
(i) AC2 markers or proteins as provided in Table 7;
(ii) AC3 markers or proteins as provided in Table 8, 10 or 11;
(iii) markers or proteins selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 as set out in Table 5;
(iv) markers or proteins selected from COL1A2, COL5A1, COL16A1, COL14A1, COL4A2, PXDN, ST5, DCLK1, SCARA5, EGFR, EGR1 and ZFHX4 as set out in Table 9;
(v) markers or proteins selected from COL14A1, DCLK1, FNDC1, COL16A1, COL1A2, KIAA1755, PXDN, and COL5A1 as set out in Table 12; and
c) comparing expression of the markers selected from a panel of RNA or protein markers in the blood sample to expression of the markers in a control blood sample to determine if expression of the markers selected from a panel of RNA or protein markers in the blood sample is increased relative to expression in the control blood sample, wherein detection of increased expression serves to predict an impending RA flare in a patient;
and treating the patient thereby diagnosed with an impending RA flare by administering a therapeutically effective amount of one or more disease-modifying agent for treating RA.

The expression, differential expression, or quantitatively increased amounts of the AC2 RNA markers or proteins may predict or may be utilized to predict an RA flare in about 2 weeks, about 14 days, or about 12-14 days, up to in about 3 weeks or about 21 days, given margin of error. The expression or quantitatively increased amounts of the AC3 RNA markers or proteins may predict or may be utilized to predict an RA flare in about one week, about 7 days, or about 5-7 days, up to or about up to two weeks or about 14 days, given margin of error. In an aspect of the method, the expression, differential expression, or quantitatively increased amounts of the markers or proteins selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 or of the markers or proteins selected from COL1A2, COL5A1, COL16A1, COL14A1, COL4A2, PXDN, ST5, DCLK1, SCARA5, EGFR, EGR1 and ZFHX4 predicts an RA flare in about one week, about 7 days, or about 5-7 days, up to about two weeks or 14 days given margin of error. In an aspect of the method, the expression, differential expression, or quantitatively increased amounts of the markers or proteins selected from COL14A1, DCLK1, FNDC1, COL16A1, COL1A2, KIAA1755, PXDN, and COL5A1 predicts an RA flare in about one week, about 7 days, or about 5-7 days, up to about two weeks or 14 days given margin of error.

Evaluation of RNA or protein expression may be conducted using any method known in the art. Thus, in accordance with the methods of the present disclosure, RNA expression may be assessed by RT PCR. RNA expression may be determined by RNA sequencing. In accordance with the method, protein expression may be assessed using specific antibodies, assessing for protein activity, utilizing protein ligands.

Cell marker expression or presence on the surface of cells in the blood in accordance with the methods of the present disclosure may be determined using standard and known methods. Cell markers may be evaluated using antibodies. Cell markers may be evaluated using FACs analysis. Cells or cell markers may be evaluated using cell sorting or single-cell assessments. Cells, including the PRIME cells of the present disclosure may be isolated using cell surface marker antibodies. Methods of isolating PRIME cells, characterized as CD45−CD31−PDPN+ cells, using or via cell surface markers are thus provided in an embodiment of the present disclosure.

The disease-modifying agent for treating RA may be selected from standard or clinically recognized agents or therapies for RA or arthritic conditions or inflammatory diseases and conditions. In aspects, the disease-modifying agent for treating RA may be one or more agent selected from a nonsteroidal anti-inflammatory drug (NSAID), steroid, methotrexate, disease-modifying antirheumatic drug (DMARDs), biologic DMARD, and oral janus kinase (JAK) inhibitor. DMARD may be one or more of methotrexate (Trexall, Otrexup), leflunomide (Arava), hydroxychloroquine (Plaquenil) and sulfasalazine (Azulfidine). Biologic DMARD may be one or more or any of abatacept (Orencia), adalimumab (Humira), anakinra (Kineret), baricitinib (Olumiant), certolizumab (Cimzia), etanercept (Enbrel), golimumab (Simponi), infliximab (Remicade), rituximab (Rituxan), sarilumab (Kevzara), tocilizumab (Actemra) and tofacitinib (Xeljanz). Exemplary JAK inhibitor include tofacitinib (Xeljanz and Xeljanz XR), baricitinib (Olumiant), and upadacitinib (Rinvoq). The biologic DMARD may be a tumor necrosis factor (TNF) inhibitor. In an aspect, the biologic DMARD may be an anti-inflammatory antibody or an antibody directed to an inflammation or immune modulatory molecule. In an aspect, the antibody may be an interleukin antibody. The antibody may be an IL-17 specific antibody or an IL-17RD specific or IL-17RD blocking or neutralizing antibody. The antibody may be a podaplanin (PDPN) antibody. The antibody may be a bispecific podaplanin (PDPN) antibody, such as a bispecific PDPN IL-17RD antibody.

A novel and unique circulating cell has been identified as a cellular indicator of an impending flare and which specifically contributes to the impending flare. Thus, a circulating pre-inflammatory mesenchymal (PRIME) cell, characterized as a CD45−CD31−PDPN+ cell, has been identified and is provided herein wherein the presence of the cell in peripheral blood is indicative or predictive of an impending RA flare. In an aspect, the PRIME cell additionally expresses IL-17RD and is IL-17RD+. In an aspect, a panel of PRIME cells additionally expresses IL-17RD and is IL-17RD+. Methods for isolating PRIME cells, CD45−CD31−PDPN+ cells, and additionally IL-17RD+ cells, including for analysis and/or evaluation with potential therapeutics or cell modulators, are provided as an embodiment of the present disclosure. The cells may be selected or isolated via their cell surface markers, including as CD45−CD31−PDPN+, additionally including IL-17RD+. Methods for evaluating agents that modulate or inhibit CD45−CD31−PDPN+ cells, and additionally IL-17RD+ cells, are provided.

A method is herein now provided for predicting an impending RA flare comprising evaluating a blood sample from a patient for the presence of a PRIME cell characterized as a CD45−CD31−PDPN+ cell, wherein the presence of detectable PRIME cells in peripheral blood in a patient predicts an impending RA flare in the patient. In an aspect thereof, the method includes further evaluating for the presence of IL-17RD on a CD45−CD31−PDPN+ cell. Methods are provided for evaluating and treating an impending flare in an RA patient comprising evaluating the peripheral blood of a patient for the presence of a PRIME cell characterized as a CD45−CD31−PDPN+ cell and treating a patient that is positive for PRIME cells in their peripheral blood with a disease-modifying agent for RA. Methods are provided for evaluating and treating an impending flare in an RA patient comprising evaluating the peripheral blood of a patient for the presence of a PRIME cell characterized as a CD45−CD31−PDPN+IL-17RD+ cell and treating a patient that is positive for PRIME cells in their peripheral blood with a disease-modifying agent for RA.

Methods are provided for evaluating and treating an impending flare in an RA patient comprising evaluating the peripheral blood of a patient for the presence of RNA(s) or protein(s) expressed, specifically expressed or particularly expressed by a PRIME cell characterized as a CD45−CD31−PDPN+ cell and treating a patient that is positive for expressing, specifically expressing or particularly expressing RNA(s) or protein(s) of PRIME cells in their peripheral blood with a disease-modifying agent for RA. Methods are provided for evaluating and treating an impending flare in an RA patient comprising evaluating the peripheral blood of a patient for the presence of RNA(s) or protein(s) expressed, specifically expressed or particularly expressed by a cell characterized as a CD45−CD31−PDPN+IL-17RD+ cell and treating a patient that is positive for the presence of RNA(s) or protein(s) expressed, specifically expressed or particularly expressed by a cell characterized as a CD45−CD31−PDPN+IL-17RD+ cell in their peripheral blood with a disease-modifying agent for RA. The specification details the overlapping expression of various and numerous specific AC3 marker genes with gene expression (for example as detected by RNA presence) in PRIME cells characterized as a CD45−CD31−PDPN+ cells.

The disease-modifying agent may be selected from those as described and provided herein or as known and recognized to a clinician or physician. Antibodies directed to immune modulators or inflammatory modulators may be selected. In an aspect, the patient is treated with an IL-17 or IL-17RD antibody. In another aspect, the patient is further treated with an anti-inflammatory agent and/or an immune modulating agent. In an aspect the patient is treated with a podaplanin (PDPN) antibody. In an aspect the patient is treated with one or more antibody directed to a surface marker on the PRIME cell, for example a marker from among the AC3 gene markers which is expressed on the cell surface. In an aspect the patient is treated with one or more antibody directed to a surface marker selected from the markers or proteins COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6. In an aspect, the patient is treated with one or more antibody directed to a surface marker selected from the markers or proteins COL14A1, DCLK1, FNDC1, COL16A1, COL1A2, KIAA1755, PXDN, and COL5A1.

Two monoclonal antibodies targeting IL-17A (Secukinumab (AIN457, Novartis), Ixekizumab (LY2439821, EliLilly) and one against the IL-17 receptor (Brodalumab (KHK4827, AMG827, Kyowa/Amgen) are approved for the treatment of moderate-to-severe plaque psoriasis (Silfvast-Kaiser A et al. (2019) Expert Opin Biol Ther 19(1):45-54; doi: 10.1080/14712598.2019.1555235). Other IL-17A antibodies include Remtolumab (ABT-122, Abbvie), ALX-0761 (MSB0010841, Ablynx/Merck), BCD-085 (Biocad), COVA322 (Covagen), LY3114062 (EliLilly), Perakizumab (RG4934, R05310074, Hoffman-LaRoche), Vunakizumab (SHR-1314, Jiangsu Hengrui), CNTO 6785 (Morphosys/Janssen), CJM112 (Novartis) and Bimekizumab (UCB4940, UCB) (Ibrahim S et al (2017) Clin Colorectal Cancer 17(1):e109-13). The IL-17A specific antibody secukinumab and other anti-IL-17 agents have also been reported effective in ankylosing spondylitis (Wendling D et al (2019) Expert Opin Biol Ther 19(1):55-64. doi: 10.1080/14712598.2019.1554053). These antibodies are of use and application in accordance with the present disclosure.

Podaplanin (PDPN) antibodies have also been described. These include the anti-podaplanin antibody clone 8.1.1(Lax S et al (2017) BMJ Open Respiratory Res 4:e000257.doi:10.11361/bmjresp-2017-000257), a chimeric mouse-human podaplanin anibody chLpMab-7 (Kato Y (2015) Oncotarget 6(34):36003-36018) and anti-human podaplanin rat antibody NZ-1 and chimeric rat-human antibody derived therefrom (NZ-8) Abe S et al (2013) J Immunol 190(12):6239-6249).

Immune modulators may be included in a composition with or administered with antibodies or agents, including those targeting the markers or proteins of the present disclosure, and/or administered at a different time to enhance immune modulation and/or RA therapy, including immune therapies directed against RA or RA flares. An immune modulator may be an adjuvant. Applicable immune modulators include IDO, TDO (Platten M (2012) Cancer Research 72(21):5435-40), Q-galactosyl ceramide and analogs thereof such as threitolceramide (ThrCer) and ThrCer 6, TLR ligands such as poly I:C (TLR3), MPL (TLR4), imiquimod (TLR7), R848 (TLR8) or CpG (TLR9), iCOS, CTLA-4, PD1, PD1 ligand, OX40 and OX40 ligand, Lag3, GITR, GITR ligand interleukins, tumor necrosis factor (TNF) or other growth factors, colony stimulating factors, T cell modulators including modulators of CD8⁺ T cells, cytokines or hormones which stimulate the immune response or reduction or elimination of cancer cells or tumors (Mellman 1(2011) Nature (480):480-489). Additional immunmodulators are small molecules, antagonist antibodies or agonist antibodies targeting the applicable immune modulators including IDO, TDO, Toll like receptor family or iCOS, CTLA-4, PD1, PD1 ligand, OX40 and OX40 ligand, interleukins, tumor necrosis factor (TNF) or other growth factors, colony stimulating factors, T cell modulators including modulators of CD8⁺ T cells, cytokines which stimulate the immune response or reduction or elimination of cancer cells or tumors. Additional immune modulators, including TLR ligands such as poly I:C (TLR3), MPL (TLR4), imiquimod (TLR7), R848 (TLR8) or CpG (TLR9) can be used, including in combination with other modulators, agents or antibodies.

The unique specificity of the markers of the present disclosure provides diagnostic and therapeutic uses to identify, characterize and target RA flares or conditions and symptoms associated with an arthritis and/or inflammatory condition, particularly prior to the appearance of clinical symptoms. In particular, markers of the present disclosure are useful in modulating arthritic or inflammatory disease, particularly RA. Markers of the present disclosure are useful in inflammatory arthritis associated disorders, particularly rheumatoid arthritis (RA). Markers may further be useful in other conditions of inflammatory arthritis, particularly psoriatic arthritis (PsA), systemic lupus erythematosus (SLE, lupus), ankylosing spondylitis (AS), and gouty arthritis (gout). In an aspect thereof, antibodies or agents targeting the RNA markers or protein markers are useful in modulating an RA flare or joint inflammation or other physical indicators and symptoms of an RA flare. The antibodies or agents have applicability in therapeutic treatment or management of RA. The antibodies or agents may further have applicability in other common inflammatory arthritis associated disorders, particularly and such as psoriatic arthritis (PsA), systemic lupus erythematosus (SLE, lupus), ankylosing spondylitis (AS), and gouty arthritis (gout). The antibodies or agents targeting the RNA markers or proteins have applicability in enhancing the therapeutic effect including the anti-rheumatic effect of traditional RA disease-modifying agents or therapy(ies).

In some embodiments, the disclosure includes a set of RNA or protein markers for evaluating and predicting an impending RA flare in a patient comprising the markers selected from:
(i) a panel of at least 20 markers from the AC2 markers provided in Table 7;
(ii) a panel of at least 20 markers from the AC3 markers provided in Table 8, 10 or 11;
(iii) markers selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 as set out in Table 5;
(iv) markers selected from COL1A2, COL5A1, COL16A1, COL14A1, COL4A2, PXDN, ST5, DCLK1, SCARA5, EGFR, EGR1 and ZFHX4 as set out in Table 9;
(v) markers selected from COL14A1, DCLK1, FNDC1, COL16A1, COL1A2, KIAA1755, PXDN, and COL5A1 as set out in Table 12.

The markers may be a panel of at least 20 of the AC2 or AC3 markers, a panel of at least 10 of the AC2 or AC3 markers. At least 4, at least 5, at least 6, at least 7, at least 8, at least 9, at least 10, at least 11, at least 12, at least 13, at least 14, least 15, at least 16, at least 17, at least 18, at least 19, at least 20, at least 25, at least 30, at least 35, at least 40, at least 45, at least 50 of the AC2 or AC3 markers may be included. Particular markers may be selected and utilized. An AC2 marker panel may comprise naïve B cell gene markers and markers of developmental pathways for naïve B cells and leukocytes. A panel of AC3 markers may comprise markers of cartilage morphogenesis, endochondral bone growth, extracellular matrix organization and sublining fibroblasts. A panel of AC3 markers may comprise markers which are expressed or differentially expressed by PRIME cells, CD45−CD31−PDPN+ or are CD45−CD31− PDPN+IL-17RD+ cells, cells precursors to sublining fibroblasts, particularly RA sublining fibroblasts.

The set of markers provided and/or utilized in accordance with the methods hereof may be one or more markers selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6. In an aspect, a set of one or more markers selected from COL1A2, COL5A1, COL16A1, COL14A1, COL4A2, PXDN, ST5, DCLK1, SCARA5, EGFR, EGR1 and ZFHX4 is provided and/or utilized in accordance with the methods hereof. In an aspect, a set of one or more markers selected from COL14A1, DCLK1, FNDC1, COL16A1, COL1A2, KIAA1755, PXDN, and COL5A1 is provided and/or utilized in accordance with the methods hereof. A set of, or one or more sets of, two or more, three or more, four or more, five or more, six or more, seven or more, eight or more, nine or more, ten or more, a dozen, at least two, at least three, at least four, at least five, at least six, at least seven, at least eight, at least nine, at least ten, a set of 5-10, a set of 3-5, a set of 5-7, a set of 3-7, a set of 5-8 selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 is provided and/or utilized in accordance with the methods hereof. A set of, or one or more sets of, two or more, three or more, four or more, five or more, six or more, seven or more, eight or more, nine or more, ten or more, a dozen, at least two, at least three, at least four, at least five, at least six, at least seven, at least eight, at least nine, at least ten, a set of 5-10, a set of 3-5, a set of 5-7, a set of 3-7, a set of 5-8 selected from COL1A2, COL5A1, COL16A1, COL14A1, COL4A2, PXDN, ST5, DCLK1, SCARA5, EGFR, EGR1 and ZFHX4 is provided and/or utilized in accordance with the methods hereof. A set of one, two, three, four five, six, seven, at least 2, at least 3, at least 4, at least 5, at least 6, at least 7 or all of COL14A1, DCLK1, FNDC1, COL16A1, COL1A2, KIAA1755, PXDN, and COL5A1 is provided and/or utilized in accordance with the methods hereof. Multiple sets may be utilized, for example a set of markers of one type or metabolic pathway, combined with a distinct set of another type or metabolic or cellular pathway or cell.

In some embodiments, the disclosure includes a set of RNA or protein markers for evaluating and predicting an impending RA flare in a patient comprising one or more markers listed in Table 10, for example, at least 5, at least 10, at least 50, at least 100 markers. In some embodiments, the panel of antecedent AC3 markers comprise or consist of 2-283 markers, e.g., 8-200, 8-180, or 10-150 markers of the markers listed in Table 10, including all markers listed in Table 12. In some embodiments, the panel comprise or consist of 65-283 markers of the markers listed in Table 10, including some or all markers listed in Table 11. In some embodiments, the panel comprise or consist of all markers listed in Table 10. In some embodiments, the panel comprise or consist of one or more markers listed in Table 11, for example, at least 5, at least 10, at least 20, at least 30 markers of the markers listed in Table 11. In some embodiments, the panel comprise or consist of one or more markers listed in Table 12. In some embodiments, the panel comprise or consist of two or more markers listed in Table 12. In some embodiments, the panel comprise or consist of three or more markers listed in Table 12. In some embodiments, the panel comprise or consist of four or more markers listed in Table 12. In some embodiments, the panel comprise or consist of five or more markers listed in Table 12. In some embodiments, the panel comprise or consist of six or more markers listed in Table 12. In some embodiments, the panel comprise or consist of seven or more markers listed in Table 12. In some embodiments, the panel comprise or consist of all markers listed in Table 12, i.e., COL14A1, DCLK1, FNDC1, COL16A1, COL1A2, KIAA1755, PXDN, and COL5AL.

The present disclosure also relates to a variety of diagnostic applications, including methods for detecting the expression of or elevated presence of any of the makers of the present disclosure, particularly the RNA markers or protein markers described and provided herein. Thus, the presence or amount of RNA or protein is evaluated. Protein may be evaluated by reference to their ability to be recognized by a specific antibody directed thereto. Peptide complexes can be identified, targeted, labeled, and/or quantitated on cells, including cell(s) in peripheral blood. Diagnostic applications include in vitro and in vivo applications well known and standard to the skilled artisan and based on the present description. Diagnostic assays and kits for in vitro assessment and evaluation of marker status or marker amounts may be utilized to diagnose, evaluate and monitor patient samples including those known to have or suspected of having arthritis, inflammatory arthritis, or RA. The assessment and evaluation of RA disease status is useful in determining the suitability of a patient for a clinical trial of a drug or for the administration of a particular therapy or disease-modifying agent, including a DMARD or an antibody, including as described herein, including combinations thereof, versus a different agent or therapy. This type of diagnostic monitoring and assessment is already in practice utilizing antibodies against the HER2 protein in breast cancer (Hercep Test, Dako Corporation), where the assay is also used to evaluate patients for antibody therapy using Herceptin. In vivo applications may include imaging of joints, including radioimaging.

### Primers Sets and Markers

In some embodiments, this disclosure provides a collection of primer pairs for amplifying the cDNAs reversely transcribed from the RA markers in a panel disclosed herein. In some embodiments, the collection comprise one or more prime pairs in Table 13.

In a further embodiment, test kits suitable for use by a medical specialist may be prepared to determine the presence or absence of aberrant, differential or increased expression of one or more or of a panel of markers described herein. One class of kits will contain at least the labeled marker or its binding partner, for instance an antibody specific thereto, and directions, of course, depending upon the method selected. In some embodiments, a kit disclosed herein comprises a collection of primer pairs for amplifying the cDNAs reversely transcribed from the RA markers in a panel disclosed herein. In some embodiments, the kit comprise one or more prime pairs in Table 13. The kit may also contain peripheral reagents such as buffers, stabilizers, etc.

Accordingly, a test kit may be prepared for the demonstration of the presence of or elevated levels of one or more marker or protein marker of an impending RA flare, comprising:
(a) a predetermined amount of at least one labeled immunochemically reactive component obtained by the direct or indirect attachment of the protein marker or a specific binding partner or antibody thereto, to a detectable label;
(b) other reagents; and
(c) directions for use of said kit.

In accordance with the above, an assay system for screening potential drugs effective to modulate an RA flare or prevent an RA flare and/or the activity of a marker or protein marker of the present disclosure may be prepared. The marker peptide or antibody thereto may be introduced into a test system, and the prospective drug may also be introduced into the resulting system cell culture, and the culture thereafter examined to observe any changes in the activity of the cells, binding of the antibody, or amount and extent of the marker due either to the addition of the prospective drug alone, or due to the effect of added quantities of a known agent(s).

The present disclosure provides a system or kit for predicting an impending RA flare comprising a set of markers as described and provided herein or a set of probes and/or antibodies for evaluating a set of markers as described and provided herein.

As an example, a kit or system may include a set of markers or a set of, primers, and/or antibodies for evaluating a set of markers selected from or for a or any combination of:
(i) a panel of at least 20 markers from the AC2 markers provided in Table 7;
(ii) a panel of at least 20 markers from the AC3 markers provided in Table 8, 10 or 11;
(iii) markers selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 as set out in Table 5;
(iv) markers selected from COL1A2, COL5A1, COL16A1, COL14A1, COL4A2, PXDN, ST5, DCLK1, SCARA5, EGFR, EGR1 and ZFHX4 as set out in Table 9;
(v) markers selected from COL14A1, DCLK1, FNDC1, COL16A1, COL1A2, KIAA1755, PXDN, and COL5A1 as set out in Table 12.

The system or kit may further comprise a means for collection of the patient's blood by fingerstick, for example, a transdermal puncture tool, microtainer blood collection tubes, a lancet (e.g., a 21 guage×1.8 mm deep lancet), and the like. In some embodiments, the system or kit may further comprise one or more controls, e.g., samples that contain one or more markers in amounts similar to those in healthy individuals. In some embodiments, the system or kit may further comprise enzymes and buffers for nucleic acid extraction and amplification.

In some embodiments, the system or kit comprises a receptacle for receiving the blood sample, for example, the fingerstick blood sample. The receptacle may be configured to hold a volume of a blood sample between 1 μl and 1 ml, for example, between 10 μl and 500 μl, between 10 μl and 300 μl, or between 20 μl and 300 μl. The receptacle may have a defined volume that is the same as a suitable volume of sample for processing and analysis by the rest of the system components.

In some embodiments, a system, or kit disclosed herein comprise one or more additional components. Non-limiting examples of an additional component include a sample transportation compartment, a sample storage compartment, a sample and/or reagent receptacle, a temperature indicator, an electronic port, a communication connection, a communication component, a sample collection component, and a housing component.

In some embodiments, systems and kits disclosed herein are handheld or tabletop, and may be conveniently employed at the point of care, for example, at home, in a school, on a battlefield, on a farm, or any other site where it would be impractical or inconvenient to visit a laboratory or clinical setting. In some instances, some, a majority of, or all components of the system or kit are housed in a single device (for example, a single handheld device) having a length, a width, and a height. In some instances the length of the single device is between 1 to 20 inches, for example, 2 to 12 inches, or 2 to 8 inches, or 3 to 6 inches. In some instances the width of single unit is between 1 to 20 inches, for example, 2 to 12 inches, or 2 to 8 inches, or 3 to 6 inches. In some instances the height of single device is between 1 to 20 inches, for example, 2 to 12 inches, or 2 to 8 inches, or 3 to 6 inches.

In some instances, a method of monitoring and predicting a rheumatoid arthritis (RA) flare or increased RA disease activity comprises obtaining a fingerstick blood sample and detecting increased amounts of a panel of antecedent RA markers with the single handheld device disclosed above. In some instances, the subject performs the obtaining by pressing his or her skin against a transdermal puncture tool of the handheld device. In some embodiments, the method further comprises detecting the amount of a panel of markers with the handheld device, comparing the amounts of the markers in the panel to the amounts of the markers in the panel in a control blood sample, wherein increased amounts indicate an impending RA flare in a patient.

Computer Implemented Methods and Systems

This invention also provides a non-transitory computer-readable medium having computer-executable instructions, which when executed, causes a processor to access data attributed to a sample from a patient, the data comprising measurements of amounts of a panel of antecedent RA markers. In some embodiments, the markers are AC3 markers. In some embodiments, the markers are AC2 markers. In some embodiments, the AC3 markers comprise one or more or all markers provided in Tables 6, 8, 9, 10, 11, or 12. In some embodiments, the panel of antecedent AC3 markers comprise or consist of 2-283 markers, e.g., 8-200, 8-180, or 10-150 markers of the markers listed in Table 10, including all markers listed in Table 12. In some embodiments, the panel comprise or consist of 65-283 markers of the markers listed in Table 10, including some or all markers listed in Table 11. In some embodiments, the panel comprise or consist of all markers listed in Table 10. In some embodiments, the panel comprise or consist of all markers listed in Table 12.

The processor, executing the instructions embodied in the computer-readable medium, determined that the patient will have an impending RA flare in about one week, about 7 days, or about 5-7 days, up to or about up to two weeks or about 14 days, given margin of error.

The non-transitory computer-readable medium may be, but is not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the non-transitory computer-readable medium any be any suitable medium, upon which the program is printed, as the program can be electronically captured, via, for instance, optical scanning of the paper or other medium, then compiled, interpreted or otherwise processed in a suitable manner if necessary, and then stored in a computer memory.

Also provided herein is a computer system for predicting, monitoring, or preventing an impending RA flare. The system may comprise a detection device that is configured to detect amounts of the antecedent RA marker panels as disclosed above. The system further comprises an analyzing device in communication with the detection device, the analyzing device comprising a variety of typical computer components, including a non-transitory computer-readable medium. The analyzing device may also comprise a database storing reference values for each of the markers used in the panel. These reference values may be the average amounts of the markers from the control blood samples. In some embodiments, the control samples may be from a population of healthy individuals. As stated above, the non-transitory computer-readable medium also hosts computer-executable instructions, when executed, causes a computer processor to access data attributed to a sample from a patient, e.g., to obtain measurements of detected amounts of the antecedent RA markers in the panel and to compare the detected amounts of the antecedent RA markers in the panel with the reference values, to determine the patient will have an impending RA flare if the detected amounts are higher than the respective reference values for the markers.

In some embodiments, the results of determination are communicated to a patient, for example, an RA patient, or a patient which is suspected of having RA or an arthritic or inflammatory disease. In some embodiments, the results of determination are communicated to a physician, who will then prescribe one or more disease-modifying agent for treating RA or inflammatory diseases and conditions as disclosed herein. In some embodiments, the results of the determination are communicated on a mobile device, computer, notepad, or other electronic device in communication with a device of the system disclosed herein. In some embodiments, the results of determination and data are communicated to a mobile application via a computer network, and the mobile application is configured to locate, encrypt, index, and/or processing information. Optionally, the mobile application provides a personalized, tailored user experience based on personal information and experience. The mobile application may also provide interactive instructions to inform user how to use the system and kits, and how to interpret, prepare for, and treat the impending RA flares. The mobile application may also provide tools for sharing and tracking information, test results, and events.

This invention thus also provides a computer-implemented method for determining an impending RA flare or increased disease activity. The method comprises detecting the amounts of one or more antecedent markers in a blood sample from a patient; comparing the detected amounts with the reference values for the markers (amounts of the markers in a control sample or control samples); and determining the patient will have an impending RA flare if detected amounts are higher than the reference values for the markers. In preferred embodiments, the method steps of comparing the amounts of the markers with the reference values and/or determining the patient will have an impending RA flare are conducted with one or more computer processors.

As will be apparent to those skilled in the art to which the present disclosure pertains, the present disclosure may be embodied in forms other than those specifically disclosed above without departing from the spirit or essential characteristics of the present disclosure. The particular embodiments of the present disclosure described above, are, therefore, to be considered as illustrative and not restrictive. The scope of the present disclosure is as set forth in the appended claims rather than being limited to the examples contained in the forgoing description

### EXEMPLARY EMBODIMENTS

This disclosure provides the following non-limiting embodiments.

Embodiment 1. A method for monitoring and predicting a rheumatoid arthritis (RA) flare in a patient comprising:
(a) isolating a blood sample from said patient;
(b) evaluating the blood sample for expression or quantitatively increased amounts of one or more sets of antecedent RNA markers, protein markers or cell markers selected from:
(i) markers or proteins selected from COL1A2, COL5A1, COL16A1, COL14A1, COL4A2, PXDN, ST5, DCLK1, SCARA5, EGFR, EGR1, ZFHX4, COL3A1, COMP, FNDC1, GALNT15, SULF1, GPX8 and IGFBP6 as set out in Table 5;
(ii) markers or proteins selected from COL1A2, COL5A1, COL16A1,
(iii) COL14A1, COL4A2, PXDN, ST5, DCLK1, SCARA5, EGFR, EGR1 and ZFHX4 as set out in Table 9;
(iv) AC2 markers or proteins as provided in Table 7;
(v) AC3 markers or proteins as provided in Table 8, 10, 11, or 12; and
(vi) cell markers CD45− CD31−PDPN+;
(c) wherein the expression or quantitatively increased amounts of the RNA markers or proteins or the presence of the cell markers predicts an impending RA flare.

Embodiment 2. The method of embodiment 1, wherein the expression or quantitatively increased amounts of the AC2 RNA markers or proteins predicts an RA flare in about 2 weeks or about 12-14 days or up to 3 weeks.

Embodiment 3. The method of embodiment 1, wherein the expression or quantitatively increased amounts of the AC3 RNA markers or proteins predicts an RA flare in about 1 week or about 5-7 days or up to 2 weeks.

Embodiment 4. The method of embodiment 1, wherein a panel of at least 20 of the AC2 or AC3 markers are evaluated.

Embodiment 5. The method of embodiment 1, wherein a panel of at least 20 of the AC2 and at least 20 of the AC3 markers are evaluated.

Embodiment 6. The method of embodiment 1, wherein a panel of at least 10 of the AC2 or AC3 markers are evaluated.

Embodiment 7. The method of embodiment 1, wherein a panel of at least 10 of the AC2 and at least 10 of the AC3 markers are evaluated.

Embodiment 8. The method of embodiment 1, wherein sublining fibroblast markers selected from the AC3 markers or proteins are evaluated.

Embodiment 9. The method of embodiment 1, wherein AC3 markers or proteins expressed by CD34+, HLADR+ and DKK3+ cells are evaluated.

Embodiment 10. The method of embodiment 1, wherein the cell marker IL1 7RD is also evaluated.

Embodiment 11. The method of embodiment 1, wherein RNA expression is assessed by RT PCR.

Embodiment 12. The method of embodiment 1 wherein protein expression is assessed using specific antibodies.

Embodiment 13. The method of embodiment 1 wherein cell markers are evaluated using FACs analysis.

Embodiment 14. The method of embodiment 1 wherein the antecedent RNA markers or protein markers or selected from:
(a) markers or proteins selected from COL1A2, COL5A1, COL16A1, COL14A1, COL4A2, PXDN, ST5, DCLK 1, SCARA5, EGFR, EGR 1, ZFHX4, COL3A1, COMP, FNDC1, GALNT15, SULF1, GPX8 and IGFBP6 as set out in Table 5;
(b) markers or proteins selected from COL1A2, COL5A1, COL16A1, COL14A1, COL4A2, PXDN, ST5, DCLK 1, SCARA5, EGFR, EGR 1 and ZFHX4 as set out in Table 9; and
(c) AC3 markers or proteins as provided in Tables 8, 10, 11, or 12; are decreased in peripheral blood during an RA flare or once a patient exhibits symptoms of an RA flare.

Embodiment 15. A method for predicting an impending RA flare and treating a flare in a patient, the method comprising:
(a) isolating a blood sample from the patient;
(b) contacting the blood sample with reagents specific for markers selected from a panel of RNA or protein markers to assess expression of the RNA or protein markers, wherein the panel of RNA or protein markers is selected from:
(i) markers or proteins selected from COL1A2, COL5A 1, COL16A1, COL14A1, COL4A2, PXDN, ST5, DCLK 1, SCARA5, EGFR, EGR1, ZFHX4, COL3A1, COMP, FNDC1, GALNT15, SULF1, GPX8 and IGFBP6 as set out in Table 5;
(ii) markers or proteins selected from COL1A2, COL5A 1, COL16A 1, COL14A 1, COL4A2, PXDN, ST5, DCLK 1, SCARA5, EGFR, EGR1 and ZFHX4 as set out in Table 9;
(iii) AC2 markers or proteins as provided in Table 7; and
(iv) AC3 markers or proteins as provided in Table 8;
(a) comparing expression of the markers selected from a panel of RNA or protein markers in the blood sample to expression of the markers in a control blood sample to determine if expression of the markers selected from a panel of RNA or protein markers in the blood sample is increased relative to expression in the control blood sample, wherein detection of increased expression serves to predict an impending RA flare in the patient;
and treating the patient thereby diagnosed with an impending RA flare by administering a therapeutically effective amount of one or more disease-modifying agent for treating RA.

Embodiment 16. The method of embodiment 15, wherein RNA expression is assessed by RT PCR.

Embodiment 17. The method of embodiment 15, wherein protein expression is assessed using specific antibodies.

Embodiment 18. The method of embodiment 15, wherein the expression or quantitatively increased amounts of the AC2 RNA markers or proteins predicts an RA flare in about 2 weeks or about 12-14 days or about 3 weeks.

Embodiment 19. The method of embodiment 15, wherein the expression or quantitatively increased amounts of RNA or protein markers selected from:
(a) markers or proteins selected from COL1A2, COL5A1, COL16A 1, COL14A1, COL4A2, PXDN, ST5, DCLK 1, SCARA5, EGFR, EGR 1, ZFHX4, COL3A1, COMP, FNDC1, GALNT15, SULF1, GPX8 and IGFBP6;
(b) markers or proteins selected from COL1A2, COL5A 1, COL16A 1, COL14A 1, COL4A2, PXDN, ST5, DCLK1, SCARA5, EGFR, EGR 1 and ZFHX4; and
(c) the AC3 RNA markers or proteins;
wherein the method predicts an RA flare will occur in about 1 week, about 5-7 days, or about 2 weeks.

Embodiment 20. The method of embodiment 15, wherein the disease-modifying agent for treating RA is one or more agent selected from a nonsteroidal anti-inflammatory drug (NSAID), steroid, methotrexate, disease-modifying antirheumatic drug (DMARDs), biologic DMARD, and oral janus kinase (JAK) inhibitor.

Embodiment 21. The method of embodiment 20, wherein the DMARD is selected from methotrexate (Trexall, Otrexup), leflunomide (Arava), hydroxychloroquine (Plaquenil) and sulfasalazine (Azulfidine).

Embodiment 22. The method of embodiment 20, wherein the biologic DMARD is selected from abatacept (Orencia), adalimumab (Humira), anakinra (Kineret), baricitinib (Olumiant), certolizumab (Cimzia), etanercept (Enbrel), golimumab (Simponi), infliximab (Remicade), rituximab (Rituxan), sarilumab (Kevzara), tocilizumab (Actemra) and tofacitinib (Xeljanz).

Embodiment 23. The method of embodiment 20, wherein the biologic DMARD is a tumor necrosis factor (TNF) inhibitor.

Embodiment 24. The method of embodiment 20, wherein the biologic DMARD is combined with an NSAID and/or with methotrexate.

Embodiment 25. The method of embodiment 20, wherein the JAK inhibitor is selected from tofacitinib (Xeljanz and Xeljanz XR), baricitinib (Olumiant), and upadacitinib (Rinvoq).

Embodiment 26. The method of embodiment 15, wherein the disease-modifying agent for treating RA is an IL-17 antibody or an IL1 7RD blocking antibody.

Embodiment 27. A circulating pre-inflammatory mesenchymal (PRIME) cell characterized as a CD45−CD31− PDPN+ cell, wherein the presence of the cell in peripheral blood is indicative or predictive of an impending RA flare.

Embodiment 28. The PRIME cell of embodiment 27, which additionally expresses IL1 7RD and is IL1 7RD+.

Embodiment 29. A method of predicting an impending RA flare comprising evaluating a blood sample from a patient for the presence of a PRIME cell characterized as a CD45−CD31−PDPN+ cell, wherein the presence of detectable PRIME cells in peripheral blood in a patient predicts an impending RA flare in the patient.

Embodiment 30. The method of embodiment 29, further evaluating for the presence of IL-17RD on a CD45−CD31− PDPN+ cell.

Embodiment 31. A method for evaluating and treating an impending flare in an RA patient comprising evaluating the peripheral blood of a patient for the presence of a PRIME cell characterized as a CD45− CD3 1−PDPN+IL1 7RD+ cell and treating a patient that is positive for PRIME cells in their peripheral blood with a disease-modifying agent for RA.

Embodiment 32. The method of embodiment 31, wherein the patient is treated with an IL-17 or IL-17RD antibody.

Embodiment 33. The method of embodiment 32, wherein the patient is further treated with an anti-inflammatory agent and/or an immune modulating agent.

Embodiment 34. A set of RNA or protein markers for evaluating and predicting an impending RA flare in a patient comprising the markers selected from:
(i) markers selected from COLIA2, COL5A1, COL16A 1, COL14A1, COL4A2, PXDN, ST5, DCLK I, SCARA5, EGFR, EGR I, ZFHX4, COL3A 1, COMP, FNDC1, GALNT15, SULF1, GPX8 and IGFBP6 as set out in Table 5;
(ii) markers selected from COLI A2, COL5A 1, COL16A1, COL14A 1, COL4A2, PXDN, ST5, DCLK1, SCARA5, EGFR, EGR 1 and ZFHX4 as set out in Table 9;
(iii) a panel of at least 20 markers from the AC2 markers provided in Table 7; and
(iv) a panel of at least 20 markers from the AC3 markers provided in Table 8.

Embodiment 35. The marker set of embodiment 34, wherein the panel of AC2 markers comprises nai:ve B cell gene markers and markers of developmental pathways for naive B cells and leukocytes.

Embodiment 36. The marker set of embodiment 34, wherein the panel of AC3 markers comprises markers of cartilage morphogenesis, endochondral bone growth, extracellular matrix organization and sublining fibroblasts.

Embodiment 37. A system or kit for predicting an impending RA flare comprising a set of markers of embodiment 34 or a set of probes and/or antibodies for evaluating a set of markers of embodiment 34.

Embodiment 38. The system or kit of embodiment 37, which further comprises a means for collection of the patient's blood by fingerstick.

Embodiment 2.1. A method for monitoring and predicting a rheumatoid arthritis (RA) flare or increased RA disease activity in a patient comprising:
(a) detecting in the blood sample increased amounts of a panel of antecedent RA markers, wherein the panel comprise one or more AC3 markers listed in Table 10;
(b) wherein the expression or quantitatively increased amounts of the AC3 markers predicts an impending RA flare or increased RA disease activity.

Embodiment 2.2. The method of embodiment 2.1, wherein the panel comprise one or more AC3 markers listed in Table 11.

Embodiment 2.3. The method of embodiment 2.1, wherein the one or more or all AC3 markers are selected from those listed in Table 12 (COL14A1, DCLK1, FNDC1, COL16A1, COL1A2, KIAA1755, PXDN, and COL5A1).

Embodiment 2.3.1 The method of claim 2.1, wherein a panel of antecedent RA markers comprising at least 2 or more markers listed in Table 12 (COL14A1, DCLK1, FNDC1, COL16A1, COL1A2, KIAA1755, PXDN, and COL5A1) are evaluated to detect increased amounts of one or more of the AC3 markers evaluated.

Embodiment 2.3.2 The method of Embodiment 2.1, wherein a panel of antecedent RA markers comprising those listed in Table 12 (COL14A1, DCLK1, FNDC1, COL16A1, COL1A2, KIAA1755, PXDN, and COL5A1) are evaluated to detect increased amounts of one or more of the AC3 markers listed in Table 12.

Embodiment 2.4. The method of any of the preceding embodiments, wherein the increased amounts of the AC3 RNA markers or the AC3 protein markers predicts an RA flare in about 1 week or about 5-7 days or up to 2 weeks.

Embodiment 2.5. The method of any one of the preceding embodiments, wherein the panel consists of 2 to 283 antecedent markers.

Embodiment 2.6. The method of any one of the preceding embodiments, wherein a panel of at least 3, at least 4, at least 5 at least 6 of the AC3 markers are evaluated.

Embodiment 2.7. The method of any one of the preceding embodiments, wherein the method further comprises detecting increased amounts of one or more AC2 markers listed in Table 7.

Embodiment 2.8. The method of any one of the preceding embodiments, wherein the increased amounts of one or more AC3 markers predicts an RA flare in about 1 week or about 5-7 days or up to 2 weeks.

Embodiment 2.9. The method of any of the preceding embodiments, wherein the increased amounts of one or more AC3 RNA markers in are detected using RNAseq or RT-PCR.

Embodiment 2.10. The method of any of Embodiment 2.1-Embodiment 2.9, wherein the detecting the increased amounts of the one or more AC3 RNA markers comprises amplifying the one or more AC3 RNA markers in Table 12 using primer listed in Table 13.

Embodiment 2.11. The method of any one of Embodiment 2.1-Embodiment 2.10, the increased amount of the one or more AC3 protein markers is detected using antibodies specific for the AC3 protein markers.

Embodiment 2.12. The method of any of Embodiment 2.1-Embodiment 2.11, wherein the amount of AC3 markers are decreased in peripheral blood during an RA flare or once a patient exhibits symptoms of an RA flare.

Embodiment 2.13. A computer implemented method for predicting an impending RA flare or increased RA disease activity in a patient comprising:
a) detecting amounts of a panel of AC3 markers, wherein the panel comprise one or more AC3 markers listed in Table 10, and
b) determining, using one or more computer processors, that the patient has an impending RA flare or increased RA disease activity if the amounts of the panel of AC3 markers are higher than the amounts of the AC3 markers in a control blood sample.

Embodiment 2.14. A method for predicting or treating an impending RA flare in a patient, the method comprising:
a) contacting a blood sample from the patient with reagents specific for a panel of AC3 markers, wherein the panel comprise one or more AC3 markers listed in Table 10,
b) detecting amounts of the markers of the panel in the blood sample, wherein detection of increased amounts serves to predict an impending RA flare in a patient,
c) comparing the amounts of the markers in the panel to the amounts of the markers in a control blood sample, and
d) administering a therapeutically effective amount of one or more disease-modifying agent for treating RA if the amounts of the markers of the panel in the blood sample is increased relative to the amounts of the markers in the control blood sample, thereby treating the impending flare in the patient.

Embodiment 2.15. The method of any of the preceding embodiments, wherein the one or more AC3 markers are selected from those listed in Table 11.

Embodiment 2.16. The method of embodiment 2.1, wherein the one or more or all AC3 markers are selected from those listed in Table 12 (COL14A1, DCLK1, FNDC1, COL16A1, COL1A2, KIAA1755, PXDN, and COL5A1).

Embodiment 2.16.1 The method of claim 2.14, wherein a panel of antecedent RA markers comprising at least 2 or more markers listed in Table 12 (COL14A1, DCLK1, FNDC1, COL16A1, COL1A2, KIAA1755, PXDN, and COL5A1) are evaluated to detect increased amounts of one or more of the AC3 markers evaluated.

Embodiment 2.16.2 The method of Embodiment 2.14, wherein a panel of antecedent RA markers comprising those listed in Table 12 (COL14A1, DCLK1, FNDC1, COL16A1, COL1A2, KIAA1755, PXDN, and COL5A1) are evaluated to detect increased amounts of one or more of the AC3 markers listed in Table 12.

Embodiment 2.17. The method of any of the preceding embodiments, wherein the increased amounts of the AC3 markers predicts an RA flare in about 1 week or about 5-7 days.

Embodiment 2.18. The method of embodiment 2.14, wherein step (d) is performed within one (1) week or within 5-7 days from the step (a).

Embodiment 2.19. The method of embodiment 2.14, wherein the disease-modifying agent for treating RA is one or more agent selected from a nonsteroidal anti-inflammatory drug (NSAID), steroid, methotrexate, disease-modifying antirheumatic drug (DMARDs), biologic DMARD, and oral janus kinase (JAK) inhibitor.

Embodiment 2.20. The method of embodiment 2.19, wherein the DMARD is selected from methotrexate (Trexall, Otrexup), leflunomide (Arava), hydroxychloroquine (Plaquenil) and sulfasalazine (Azulfidine).

Embodiment 2.21. The method of embodiment 2.20, wherein the biologic DMARD is selected from abatacept (Orencia), adalimumab (Humira), anakinra (Kineret), baricitinib (Olumiant), certolizumab (Cimzia), etanercept (Enbrel), golimumab (Simponi), infliximab (Remicade), rituximab (Rituxan), sarilumab (Kevzara), tocilizumab (Actemra) and tofacitinib (Xeljanz).

Embodiment 2.22. The method of embodiment 2.20, wherein the biologic DMARD is a tumor necrosis factor (TNF) inhibitor.

Embodiment 2.23. The method of embodiment 2.20, wherein the biologic DMARD is combined with an NSAID and/or with methotrexate.

Embodiment 2.24. The method of embodiment 2.20, wherein the JAK inhibitor is selected from tofacitinib (Xeljanz and Xeljanz XR), baricitinib (Olumiant), and upadacitinib (Rinvoq).

Embodiment 2.25. The method of embodiment 2.14, wherein the disease-modifying agent for treating RA is an IL-17 antibody or an IL-17RD blocking antibody.

Embodiment 2.26. A method of treating a patient having an impending RA flare or increased RA disease activity, the method comprising
(a) selecting a patient who has been diagnosed as having increased amounts of a panel of markers as compared to a control blood sample,
wherein the panel of markers comprise one or more AC3 markers as listed in Table 10 and/or one or more AC2 markers listed in Table 7, and
(b) administering to the patient a therapeutically effective amount of one or more disease-modifying agent prior to the onset of RA.

Embodiment 2.27. The method of any of the preceding embodiments, wherein the panel of markers comprise one or more AC3 markers as listed in Table 11.

Embodiment 2.28. The method of any of the preceding embodiments, wherein the panel of markers comprises one or more AC3 markers as listed in Table 12.

Embodiment 2.29. A panel of AC3 markers for evaluating and predicting an impending RA flare or increased RA disease activity in a patient comprising the markers selected from one or more antecedent RNA markers or protein markers listed in Table 10, or Table 11, or Table 12.

Embodiment 2.30. A collection of primer pairs for amplifying the AC3 markers in embodiment 2.29.

Embodiment 2.31. The collection of primer pairs for amplifying the AC3 markers in Table 12, wherein the collection of primers comprise one or more of the following:
i) a primer pair for amplifying COL14A1, wherein the primer pair comprise oligonucleotides having sequences of SEQ ID NO: 17 and 18;
ii) a primer pair for amplifying DCLK1, wherein the primer pair comprise oligonucleotides having sequences of SEQ ID NO19 and 20;
iii) a primer pair for amplifying FNDC1, wherein the primer pair comprise oligonucleotides having sequences of SEQ ID NO:21 and 22;
iv) a primer pair for amplifying COL16A1, wherein the primer pair comprise oligonucleotides having sequences of SEQ ID NO:23 and 24; and
v) a primer pair for amplifying COL1A2, wherein the primer pair comprise oligonucleotides having sequences of SEQ ID NO:25 and 26.

Embodiment 2.32. A system or kit for predicting an impending RA flare or increased RA disease activity comprising a set of markers of embodiment 29, or a set of primers and/or antibodies for evaluating a set of markers of embodiment 29.

Embodiment 2.33. The system or kit of embodiment 2.32, which further comprises a means for collecting the patient's blood by fingerstick.

Embodiment 3.1 A method for monitoring a patient for increased probability of a rheumatoid arthritis (RA) flare or increased RA disease activity comprising: (a) detecting in a blood sample expression or amounts of a panel of markers, wherein the panel comprises one or more AC3 markers listed in Table 10 or Table 11, wherein the changes in expression or amounts of the AC3 markers predict an impending RA flare or increased RA disease activity.

Embodiment 3.2 The method of Embodiment 3.1, wherein the monitoring leads to a prediction of impending rheumatoid arthritis (RA) flare or increased RA disease activity.

Embodiment 3.3 The method of Embodiment 3.2, wherein the expression or amounts of the panel of markers are increased.

The present disclosure may be better understood by reference to the following non-limiting Examples, which are provided as exemplary of the present disclosure. The following examples are presented in order to more fully illustrate the preferred embodiments of the present disclosure and should in no way be construed, however, as limiting the broad scope of the present disclosure.

### Example 1

Longitudinal Genomics Identifies PRIME cells as Antecedents of Rheumatoid Arthritis Flares

Rheumatoid arthritis (RA), like many inflammatory diseases, is characterized by episodes of quiescence and exacerbation (flares). The molecular events leading to flares are unknown. We established a clinical and technical protocol for repeated home blood collection in RA patients to allow for longitudinal RNA sequencing (RNAseq). Samples were obtained from 364 time points from eight flares over four years in our index patient, and 235 time points from flares in three additional patients. We identified transcripts that were differentially expressed antecedent to flares and compared these to synovial single-cell RNAseq (scRNAseq). Flow cytometry and sorted blood cell RNAseq in additional RA patients were used to validate the findings.

Consistent changes were observed in blood transcriptional profiles one to two weeks antecedent to RA flare. B cell activation was followed by expansion of a previously unexplored circulating CD45−/CD31−/PDPN+, PRe-Inflammatory MEsenchymal (“PRIME”) cell in RA patient blood, which shared features of inflammatory synovial fibroblasts. Circulating PRIME cells decreased during flares from all four patients, and flow cytometry and sorted cell RNAseq confirmed the presence of PRIME cells in 19 additional RA patients.

Longitudinal genomic analysis of RA flares reveals PRIME cells in RA blood, and suggests a model in which they become activated by B cells in the weeks prior to RA flare, and then migrate out of the blood to the synovium. Longitudinal RNAseq analysis can be used to reveal dynamic changes leading to flares of chronic inflammatory disease.

Rheumatoid arthritis (RA) symptoms are highly dynamic, with stable periods interrupted by unpredictable flares of disease activity. Such waxing/waning clinical courses are characteristic of many autoimmune diseases, including multiple sclerosis (1), systemic lupus erythematosus (2), and inflammatory bowel disease (3,4), underscoring a need to develop approaches to understand what triggers transitions from quiescence to flare in autoimmune disease.

This study explores disease pathophysiology with a longitudinal, prospective analysis of blood transcriptional profiles in individual RA patients over time. Previous microarray studies of RA blood samples from relatively sparse time series data have identified few significant gene changes associated with disease activity (5-8). Here we provide the first RA study to look for molecular changes in blood that anticipate clinical flares. To do so we optimized methods by which RA patients themselves could collect high quality fingerstick blood samples for RNA sequencing (RNAseq), facilitating weekly blood sampling for months to years.

We analyzed patient reports of clinical disease activity and RNAseq data from four patients across multiple clinical flares. In our most deeply studied index case, we assessed 364 time points by RAPID3 from eight flares over four years, and analyzed 84 time points assessed by RNAseq. Collecting samples longitudinally enabled a search for transcriptional signatures that preceded clinical symptoms. Comparing these blood RNA profiles to synovial single-cell RNAseq (scRNAseq) data (9) provided evidence that a biologically coherent set of transcripts are significantly increased in the blood prior to symptom onset, and a panel of these decrease as the patients begin to experience symptoms. These latter transcripts overlap with and likely demarcate cellular precursors to a novel panel of synovial sublining fibroblast cell types detected in inflamed RA synovium using scRNAseq. Analysis in 19 additional RA patients corroborated our findings. Our data suggests a model in which a previously unexplored circulating mesenchymal cell type, detectable in the weeks prior to RA flare, becomes activated by B cells and subsequently leaves the blood, traffics to synovium, and contributes to disease activity.

### Methods

Patient Data

All patients met American College of Rheumatology/European League Against Rheumatism 2010 (10,11) criteria for RA and were seropositive for cyclized citrullinated protein antibody (CCP). Disease activity was assessed from home each week, or up to 4 times daily during escalation of flares, using the routine assessment of patient index data 3 (RAPID3) questionnaire (12). Disease activity was also assessed at clinic visits, each month, and during flares, using both the RAPID3 and the disease activity score 28 (DAS28), which incorporates tenderness and swelling from 28 joints, erythrocyte sedimentation rate (ESR) and patient global assessment of disease activity. Complete blood counts (CBC) including white blood cells (WBC), neutrophils, monocytes, lymphocytes, and platelets were performed by the clinical lab at Memorial Sloan Kettering Cancer Center. We collected 43 clinic visits from the index patient, and 25, 14 and 12 clinic visits for the other three patients studied longitudinally. Nineteen additional seropositive RA patients and 18 age and sex matched non-RA patients, for whom peripheral blood mononuclear cells (PBMC) were available, were also studied for the presence of PRIME cells by FACS and RNAseq analysis.

RNA Preparation from Fingerstick Blood

Patients self-performed fingersticks at home to collect three drops of blood into a microtainer tube prefilled with fixative, and samples were mailed overnight each week. RNA was extracted using the PAXgene® RNA kit and purified per manufacturer's protocols, except the volume of all washes and elutions was decreased to 25% of the recommended volume by the manufacturer. RNA was assessed using the Agilent BioAnalyzer for quantity and quality. For library preparation, we used the GlobinZero kit (EpiCentre #GZG1224) and Illumina's Truseq mRNA Stranded Library kit, with 11-12 PCR cycles for 5-8 nM input and sequenced on HiSeq2500 with 150 base paired-end reads. Reads were aligned to Gencodev18 using STAR and quantified using featureCounts (v1.5.0-p2). Samples with at least four million paired-end reads were retained for analysis.

Data Analysis:

Comparison of Disease Activity Measures

To describe the bivariate relationship of disease activity with RAPID3, we used the locally weighted scatterplot smoothing (LOWESS) technique. R² were calculated to assess correlations of CBC counts inferred from CIBERSORTx and counts measured by clinical labs. Inferred CIBERSORTx lymphocyte counts were the sum of B cells naive+B cells memory+T cells CD8+T cells CD4 naive+T cells CD4 memory resting+T cells CD4 memory activated. Monocytes, Macrophages M0, Macrophages M1, and Macrophages M2 were summed to infer CIBERSORTx monocyte counts. One-way ANOVA was used to test for significant differences among various clinical features according to disease activity state.

Differential Expression Analyses Across Patients

Samples were labeled “baseline” (stable RAPID3), “flare” (RAPID3 scores rose over two standard deviations above the baseline mean), or “steroid”. EdgeR (v3.24.3) (13) was used to analyze flare vs baseline differential gene expression. Permutation test (n=1×106) was used to test for the significance of overlap between genes decreased in flares in the index patient and patients 2, 3, and 4. GO enrichment (goana, from limma v3.38.3) (14) was used to identify enriched pathways in significantly differentially expressed genes in the index patient (FDR<0.1) and consistent in the direction of expression in both the index and replication patients (i.e., log fold change either both positive or both negative).

Time Series Analysis of Index Patient

We performed longitudinal data analysis on the index patient using ImpulseDE2 (v1.8.0) (15). Flare onset was defined clinically (as above) and samples from 8 weeks prior to flare up to 4 weeks after flare were analyzed (excluding any samples during which the patient was taking steroids, n=65 samples). The date of library preparation was included in the model for batch correction, and the genefilter (v1.64.0) package (16) was used to filter out lowly expressed genes.

Identification and Characterization of Coexpressed Gene Modules

We hierarchically clustered mean expression of significantly differentially expressed genes identified in the ImpulseDE2 analysis by week to flare initiation (batch corrected logrpkm expression values were calculated using edgeR) and identified five coexpressed gene modules (Clusters 1-5). We analyzed these five modules for GO term enrichment (goana).

To compare differentially expressed gene modules and to further characterize expression patterns in gene modules over time, for each module, the mean expression level for each gene was calculated across flares per week, then normalized across weeks. ABIS (17) and CIBERSORTx (18) were used to deconvolute gene expression data. To aggregate a given cluster of genes or cell type with gene markers, the mean of standardized gene expression scores or deconvolved cell type scores, respectively, within each week were plotted. To identify synovial scRNAseq cluster specific marker gene signatures, we used a previously published dataset (18) to compare the cells from one scRNAseq cluster with cells from all the other scRNAseq clusters using the single-cell RNA-seq log 2(CPM+1) matrix. We generated lists of the top 200 marker genes for each cluster using the criteria of 1) log 2FC greater than 1, 2) auc greater than 0.6, and 3) percent of expressing cells greater than 0.4. We used Fisher's exact test to evaluate enrichment of synovial cell subtype marker genes in the 5 coexpressed gene modules. P-values were corrected for multiple hypothesis test correction using the Benjamini-Hochberg procedure.

Flow Cytometry and Sorting

To assess percentages of PRIME cells in peripheral blood mononuclear cells, samples from PBMC were stained with antibodies to: CD31-APC, (WM59), Mouse IgG1-APC (MOPC-21), PDPN-PerCP (NZ1.3), Rat IgG2a (eBR2a)-PerCP, CD45-PE (HI30), Mouse IgG1-PE (MOPC-21) and TO-PRO®-3 and analyzed on BD-FACSCalibur using FlowJo 10.6.1. To flow sort and sequence PRIME cells, 20-100 Million cells from CD14-depleted leukapheresates were stained with CD31-APC(WM59), Mouse IgG1-APC(MOPC-21), PDPN-PerCP(NZ1.3), Rat IgG2a-PerCP(eBR2a), CD45-FITC(HI30), Mouse IgG1-FITC(MOPC-21), and DAPI (4′,6-Diamidino-2-Phenylindole, Dihydrochloride) and sorted on a BD FACSAria II. Illumina Stranded TruSeq library kit was used to generate cDNA libraries that were sequenced on MiSeq. DESeq2 (v1.24.0) (19) was used for differential expression analysis.

Statistics

R2 and Pearson correlation coefficients were calculated to assess the bivariate linear fit of disease activity measured by RAPID3 and DAS28 as well as CBC counts inferred from CIBERSORT cell counts and counts measured by clinical labs. Inferred CIBERSORTx lymphocyte counts were the sum of B cells naive+B cells memory+T cells CD8+T cells CD4 naive+T cells CD4 memory resting+T cells CD4 memory activated. One way ANOVA was used to test for significant differences among various clinical features according to disease activity state. Monocytes, Macrophages M0, Macrophages M1, and Macrophages M2 were summed to infer CIBERSORTx monocytes.

### Results

Clinical Protocol Development

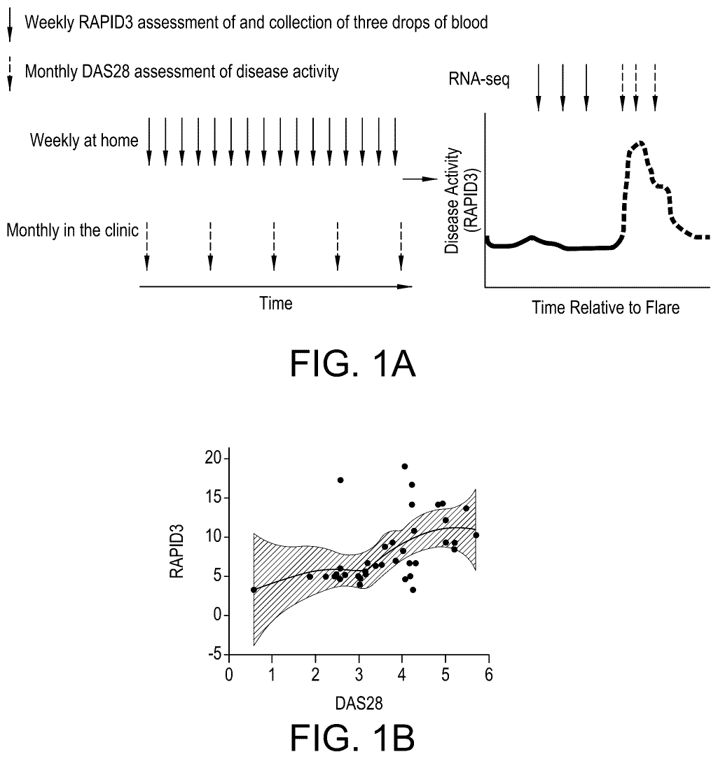

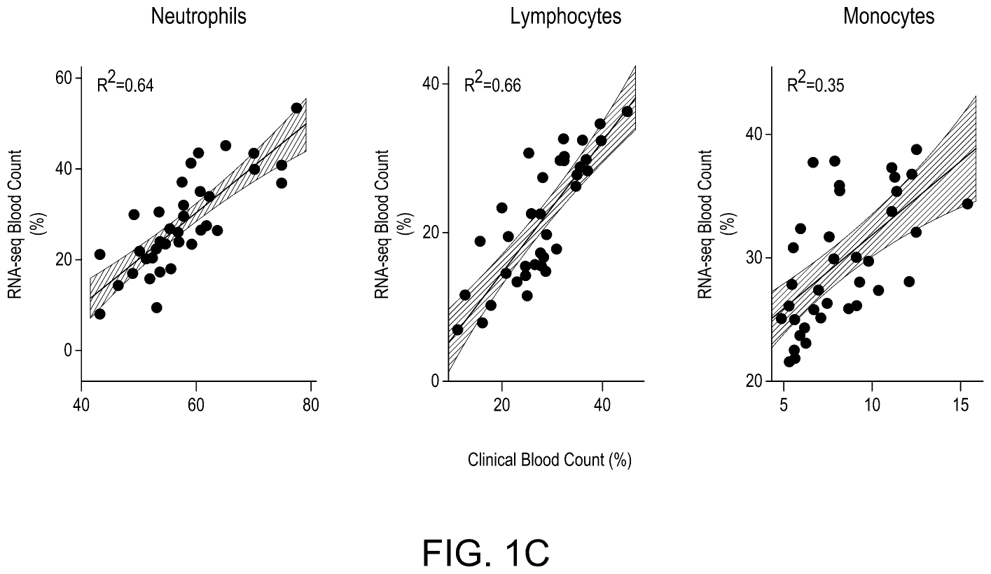

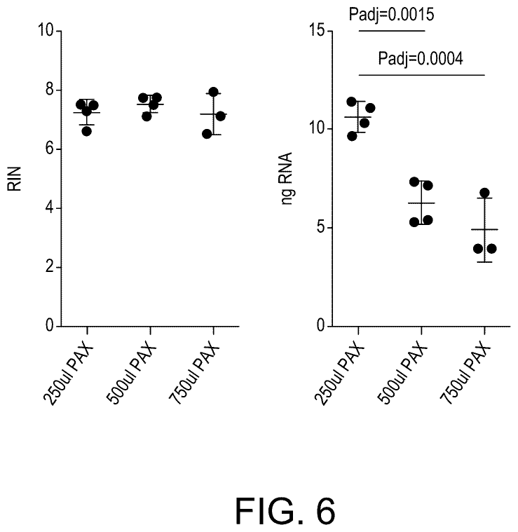

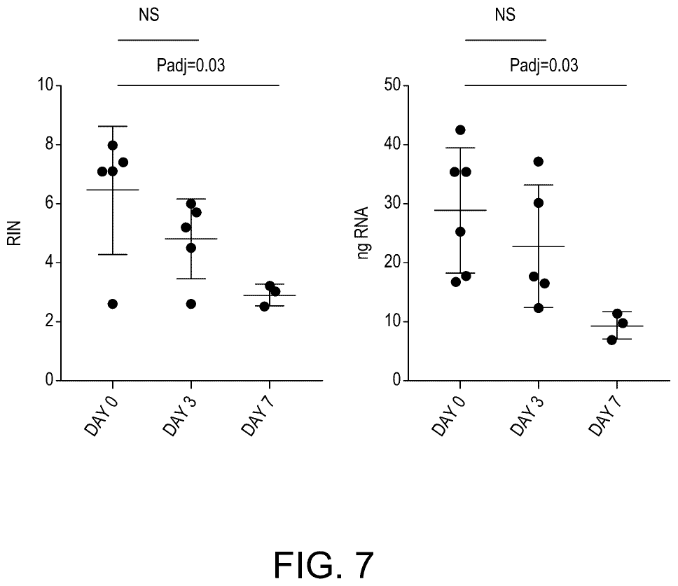

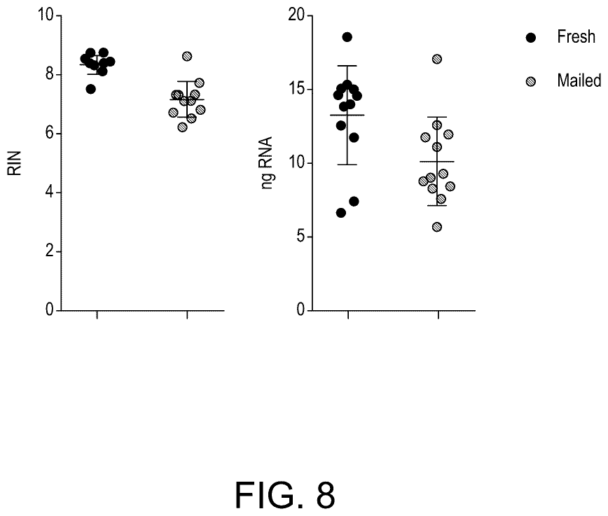

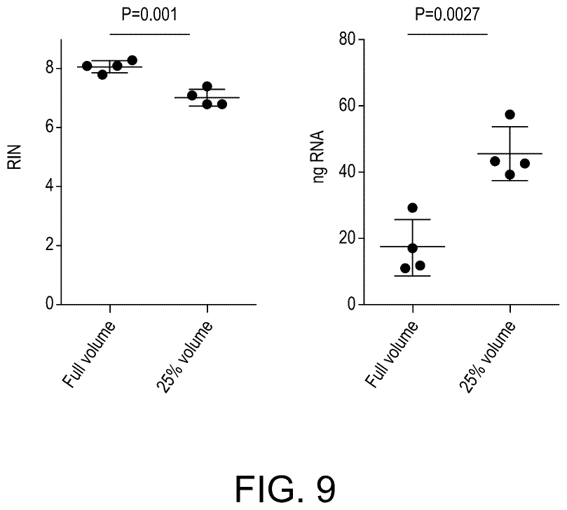

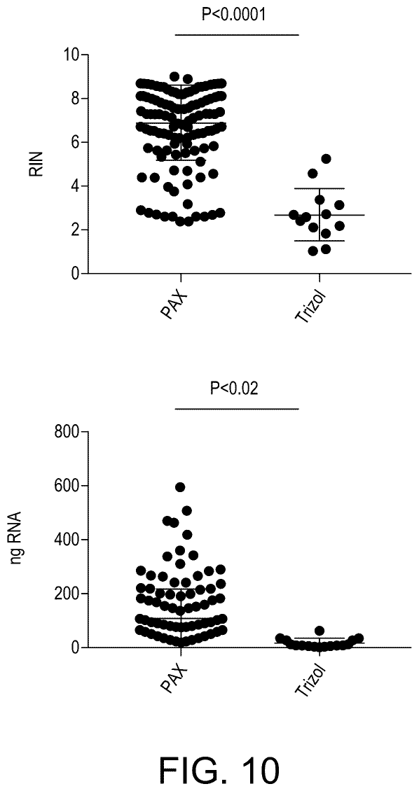

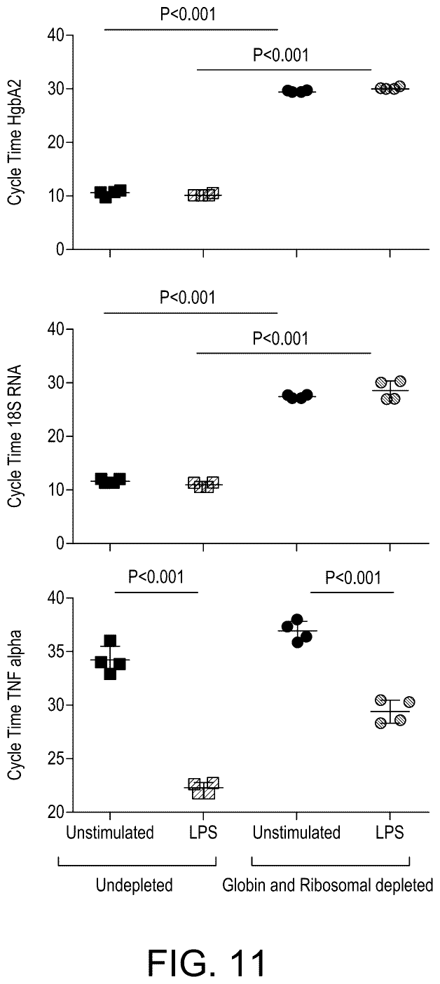

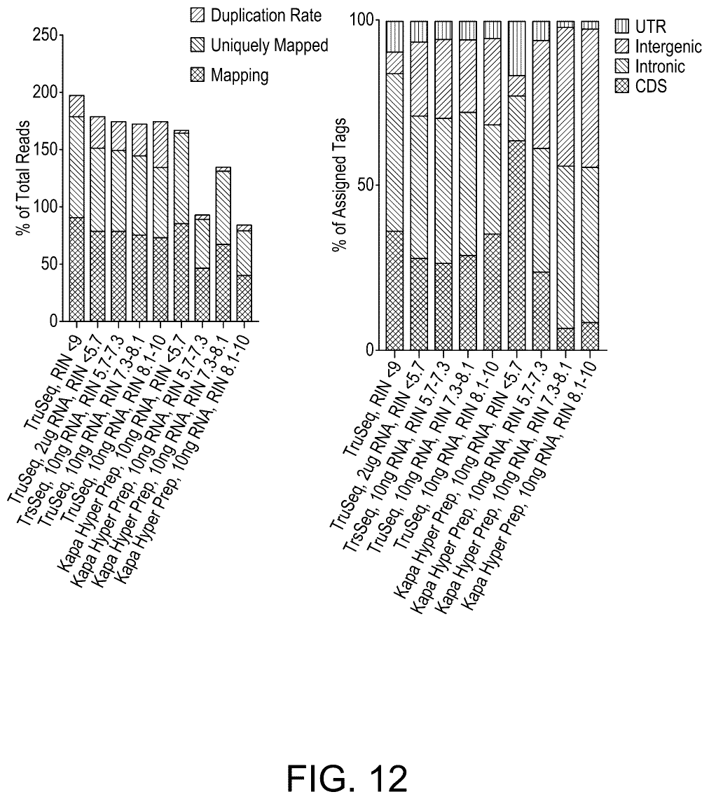

We developed strategies for home blood collection that would allow high quality and quantity RNA for sequencing (FIGS. 6-12; 15-50 ng RNA; RNA integrity (RIN) scores (mean 6.9+/−standard deviation 1.7). Study patients also documented disease activity (RAPID3 questionnaires). Four RA patients were followed for one to four years with weekly home collection of fingerstick blood samples coupled with completion of RAPID3 and monthly clinic visits, where DAS28 were collected (FIG. 1A). RNA was sequenced from a total of 189 fingerstick blood samples from 4 patients, of which 162 (87%) passed quality control filtering.

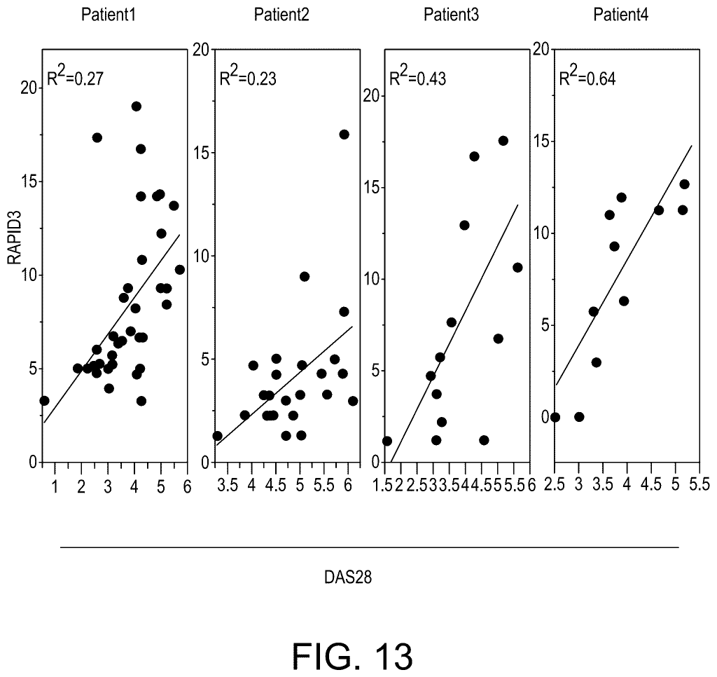

To assess the validity of patient reported disease activity, we compared their RAPID3 scores with clinician collected DAS28. Significant correlations were evident between RAPID3 and DAS28 for each of the four patients (FIG. 1B and FIG. 13). To assess the validity of fingerstick blood data, we compared RNAseq inferred white blood cell counts with clinical laboratory measurements of complete blood counts and again observed significant correlations (FIG. 1C), suggesting RNAseq of fingerstick blood was of sufficient quality to provide information that correlated with gold standard clinical measurements of blood counts. Taken together, these data indicate that patient reports of disease activity paired with fingerstick blood samples provide a high quality and robust means by which individuals can participate in longitudinal clinical research studies.

Clinical and Molecular Features of RA Flare Compared to Baseline

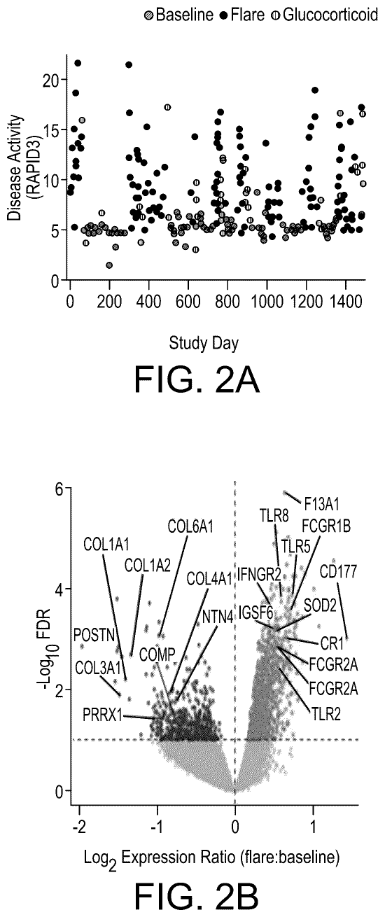

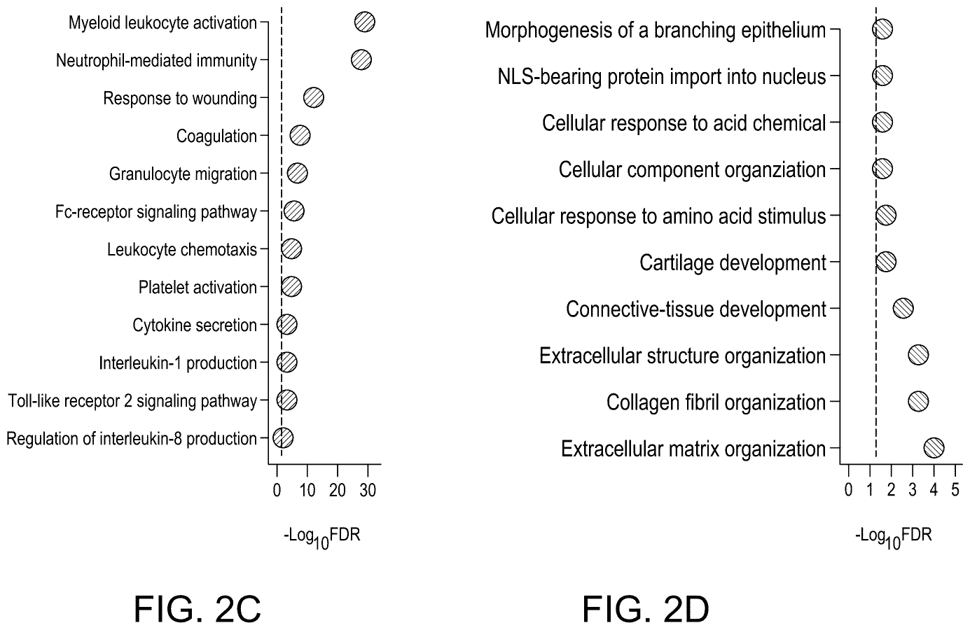

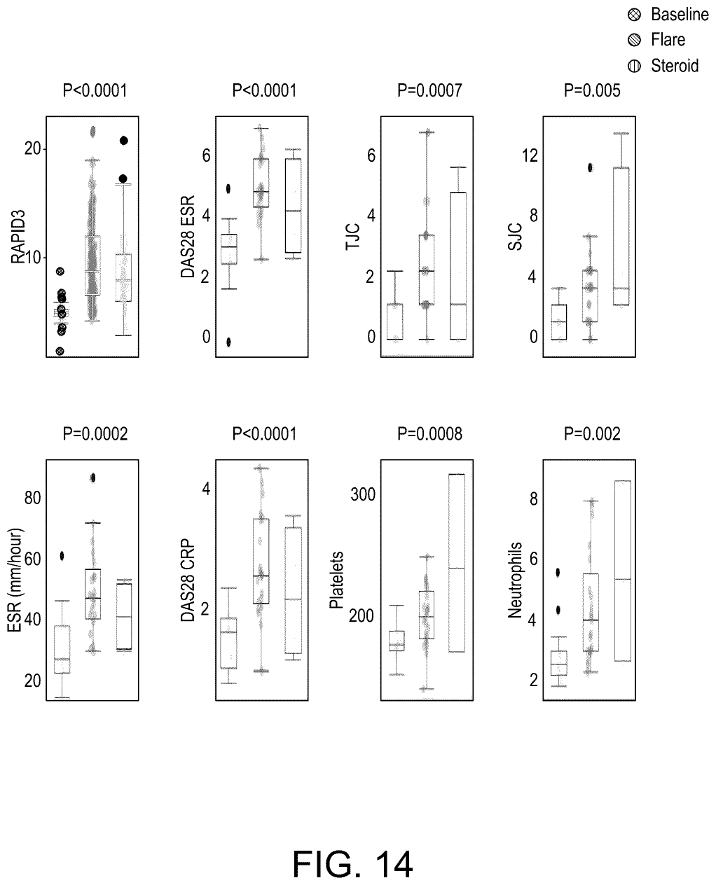

Flares were associated with increases in objective clinical and laboratory measures of RA related disease activity in the index patient (FIG. 2A and FIG. 14). Fingerstick RNAseq identified 2613 genes differentially expressed at flare versus baseline (FDR<0.1), with 1437 increased during flare (log FC>0; FIG. 2B and Table 1).

TABLE 1

| | | |
|---|---|---|
| Genes differentially expressed | FDR < 0.1 | 2613 genes |
| at flare vs baseline | | |
| Genes increased during flare | logFC > 0 | 1437 genes |

Pathway analysis identified enrichment in myeloid, neutrophil, Fc receptor signaling and platelet activation (FIG. 2C and Table 2), consistent with clinical blood count measurements during flares (FIG. 14). Interestingly, 1176 genes were significantly decreased during flare, and pathway analysis of these genes were enriched for extracellular matrix, collagen and connective tissue development (FIG. 2D and Table 2).

Time Series Analysis of Molecular Events Leading to RA Flares

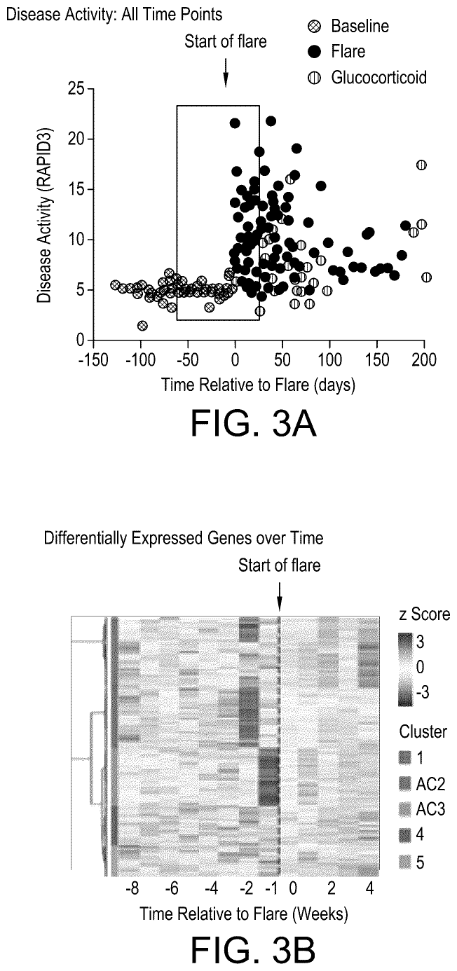

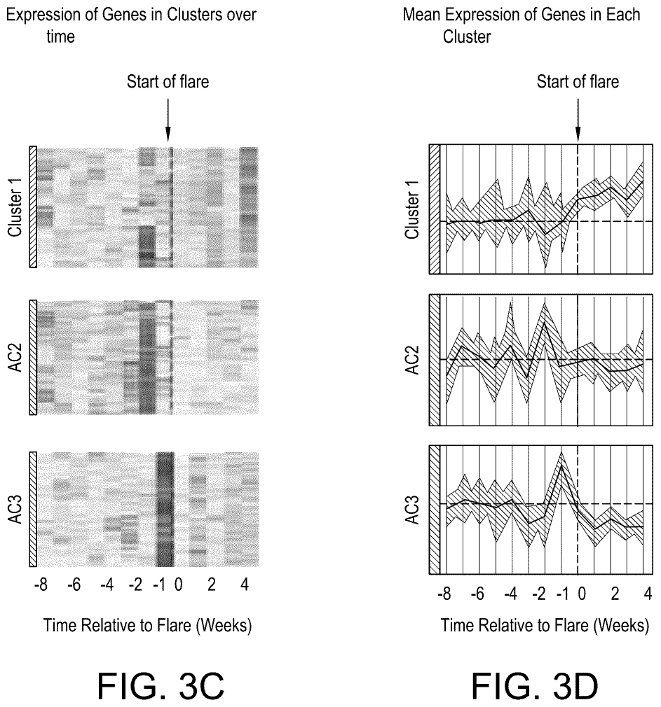

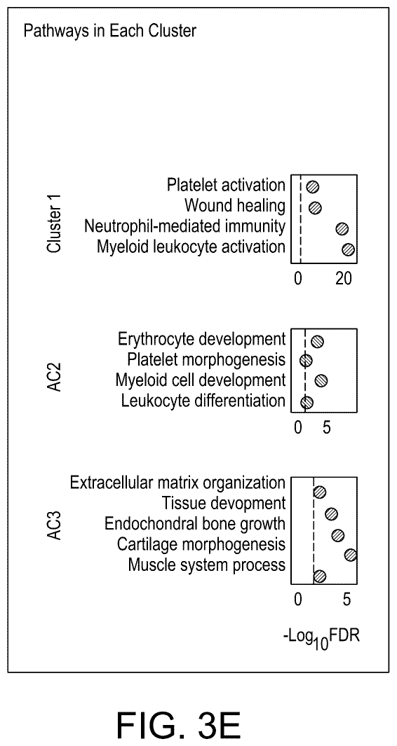

To analyze the trajectories of gene expression over time and identify potential antecedents to flare, we performed time series analysis of the RNAseq data (FIG. 3A). Notably, disease activity scores in the weeks just prior to flare were the same as baseline scores two months prior to flare, underscoring the challenges of identifying both a time frame and gene expression signature that is antecedent to flare. We focused the analysis on 65 samples acquired 8 weeks prior to flare and 4 weeks after flare initiation, binning samples according to the week they were drawn. This identified 2791 genes with significant differential expression over a period of time relative to flare (FDR<0.05), and hierarchical clustering of gene expression identified five clusters (FIG. 3B and Table 3).

TABLE 3
Pathway Analysis of Differentially Expressed Genes

| | |
|---|---|
| 27,775 genes analyzed | 2,791 genes with significant differential |
| | expression over time to flare (FDR < 0.5) |

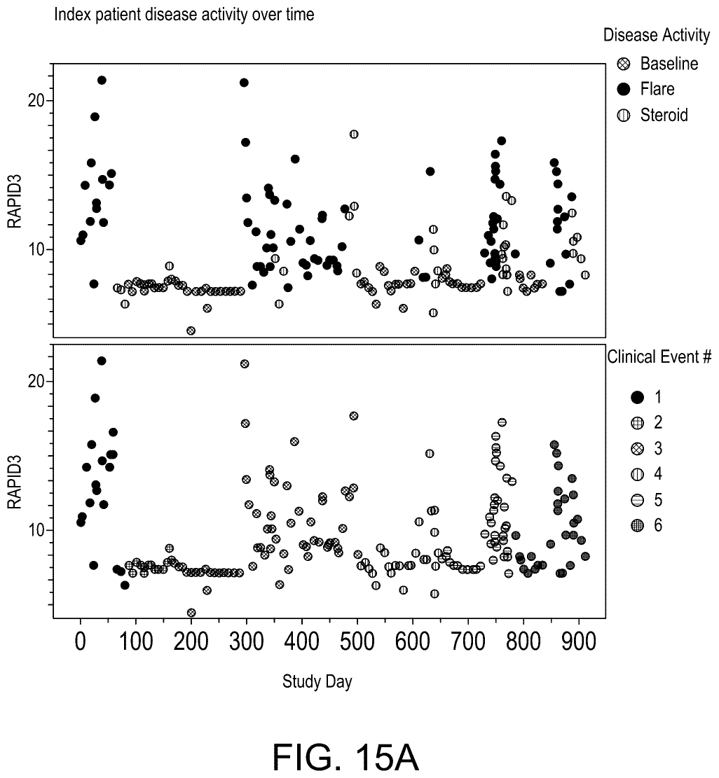

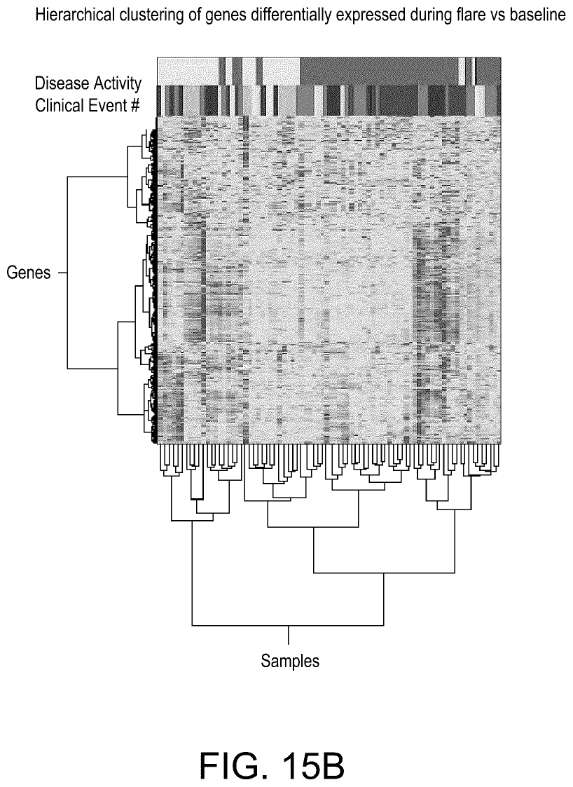

Cluster 1 represented a group of genes which increased after symptom onset (FIGS. 3C and D) and was highly overlapping (FIG. 3E) with genes increased in the flare versus baseline analysis (FIG. 2B). These gene expression clusters were reproducibly altered in 5 separate clinical flare events (FIG. 15).

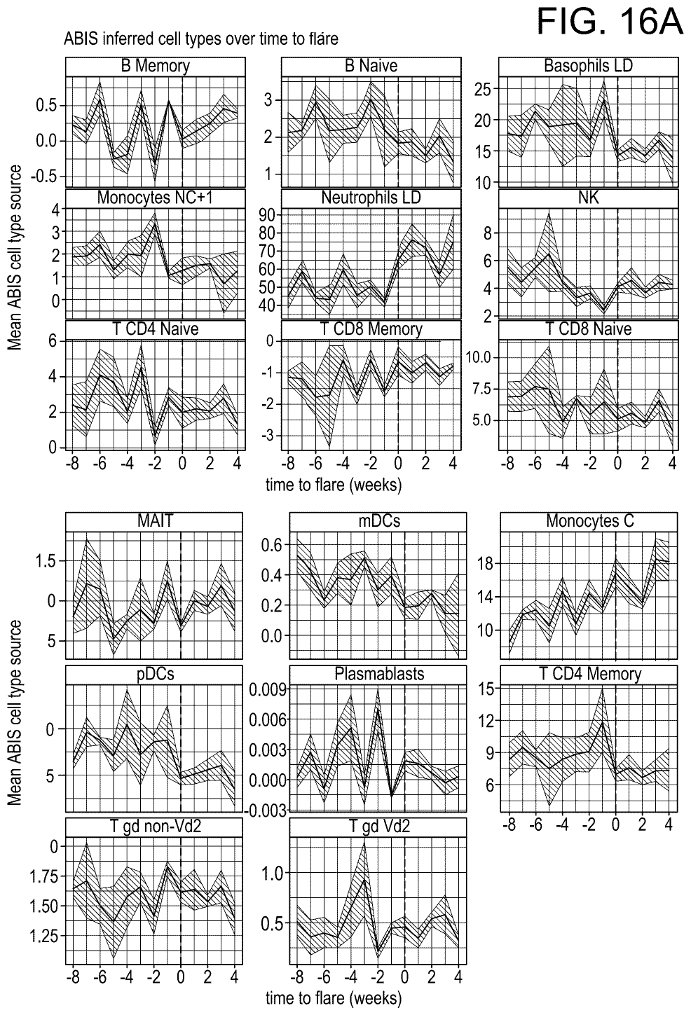

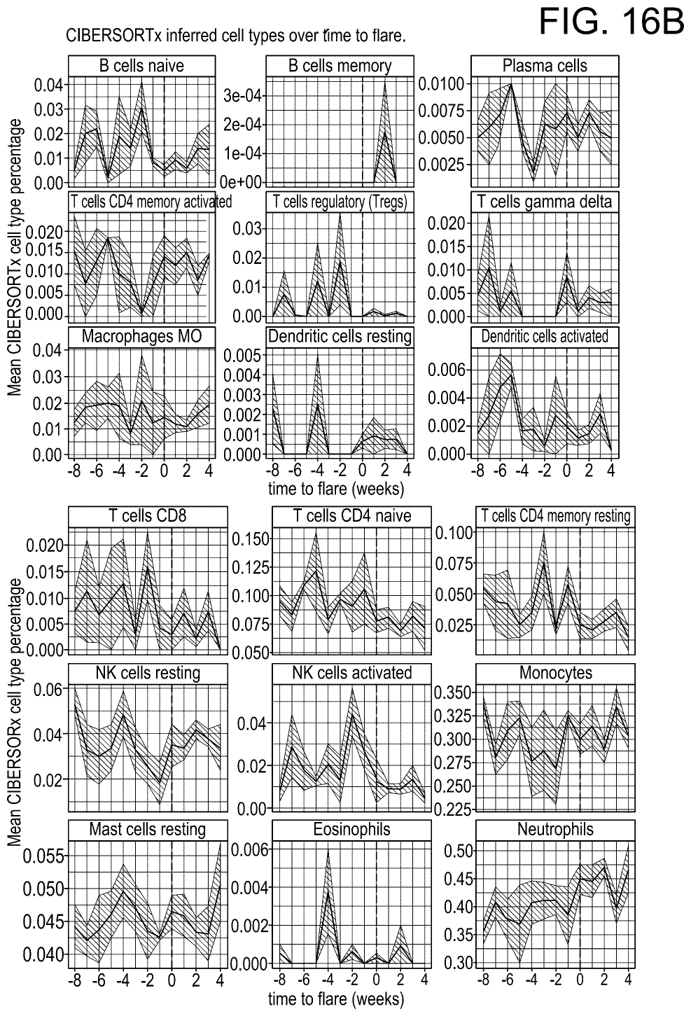

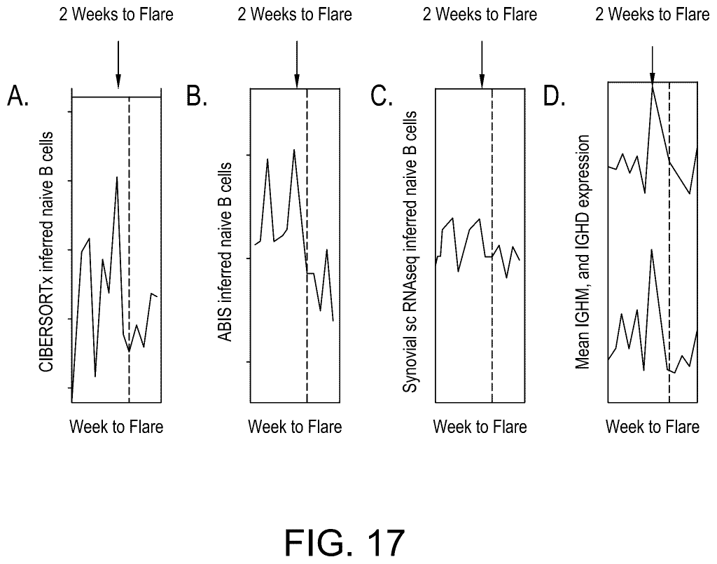

We further focused on two clusters that were differentially expressed antecedent to flare (FIG. 3C-D). Antecedent cluster 2 (AC2) transcripts increased two weeks prior to flare and were enriched with developmental pathways for naive B cells and leukocytes. Two additional means of deconvoluting the RNAseq data, CIBERSORTx and ABIS, independently confirmed evidence of B cell and T cell populations antecedent to flare, and all analyses showed evidence of innate inflammatory signatures (neutrophils and monocytes) during flare (FIGS. 16-17).

Antecedent cluster 3 (AC3) transcripts increased the week prior to flare and then decreased for the duration of flare (FIGS. 3C and D). AC3 was enriched for pathways not typical of blood samples, including cartilage morphogenesis, endochondral bone growth, and extracellular matrix organization (FIG. 3E and Table 4), suggesting the presence of an uncharacterized cell type, a mesenchymal cell.

TABLE 4

| go.id | Term | N | fdr.DE1 | fdr.DE2 | fdr.DE3 | fdr.DE4 | fdr.DE5 |
|---|---|---|---|---|---|---|---|
| GO:0032501 | multicellular | 7510 | 0.33779658 | 0.996446813 | 1.90E−13 | 0.18155513 | 0.996446813 |
| | organismal process | | | | | | |
| GO:0009887 | animal organ | 982 | 1 | 1 | 2.60E−09 | 0.426866243 | 1 |
| | morphogenesis | | | | | | |
| GO:0009653 | anatomical structure | 2604 | 0.017009962 | 0.739725387 | 3.66E−08 | 0.089707123 | 0.890858017 |
| | morphogenesis | | | | | | |
| GO:0048468 | cell development | 2093 | 0.325922506 | 0.824909599 | 4.99E−08 | 0.826157616 | 1 |
| GO:0007275 | multicellular organism | 5330 | 0.646941518 | 0.106039186 | 4.42E−07 | 0.016979265 | 0.129190893 |
| | development | | | | | | |
| GO:0048856 | anatomical structure | 5810 | 0.111685048 | 0.184216961 | 5.65E−07 | 0.026184522 | 0.332210519 |
| | development | | | | | | |
| GO:0030154 | cell differentiation | 4100 | 0.314553949 | 0.088217999 | 6.95E−07 | 0.437802459 | 0.771708045 |
| GO:0032502 | developmental | 6212 | 0.143287026 | 0.135556704 | 7.02E−07 | 0.017411509 | 0.228640051 |
| | process | | | | | | |
| GO:0007399 | nervous system | 2296 | 0.818972317 | 0.660623511 | 1.57E−06 | 0.179563884 | 1 |
| | development | | | | | | |
| GO:0043269 | regulation of ion | 660 | 1 | 1 | 1.82E−06 | 1 | 1 |
| | transport | | | | | | |
| GO:0048869 | cellular | 4282 | 0.131591479 | 0.07488625 | 3.00E−06 | 0.474081007 | 0.996446813 |
| | developmental | | | | | | |
| | process | | | | | | |
| GO:0060536 | cartilage | 29 | 1 | 1 | 3.34E−06 | 1 | 1 |
| | morphogenesis | | | | | | |
| GO:0048731 | system development | 4783 | 0.739725387 | 0.088328136 | 3.53E−06 | 0.025908171 | 0.266607283 |
| GO:0034220 | ion transmembrane | 1122 | 1 | 1 | 3.55E−06 | 1 | 1 |
| | transport | | | | | | |
| GO:0048729 | tissue morphogenesis | 612 | 0.996446813 | 1 | 6.32E−06 | 1 | 1 |
| GO:0006811 | ion transport | 1638 | 1 | 1 | 6.53E−06 | 1 | 1 |
| GO:0006812 | cation transport | 1128 | 1 | 1 | 9.04E−06 | 1 | 1 |
| GO:0098660 | inorganic ion | 824 | 1 | 1 | 9.45E−06 | 1 | 1 |
| | transmembrane | | | | | | |
| | transport | | | | | | |
| GO:0003008 | system process | 2148 | 1 | 1 | 9.57E−06 | 1 | 1 |
| GO:0034765 | regulation of ion | 454 | 1 | 1 | 1.87E−05 | 1 | 1 |
| | transmembrane | | | | | | |
| | transport | | | | | | |
| GO:0003414 | chondrocyte | 18 | 1 | 1 | 1.93E−05 | 1 | 1 |
| | morphogenesis | | | | | | |
| | involved in | | | | | | |
| | endochondral bone | | | | | | |
| | morphogenesis | | | | | | |
| GO:0003429 | growth plate cartilage | 18 | 1 | 1 | 1.93E−05 | 1 | 1 |
| | chondrocyte | | | | | | |
| | morphogenesis | | | | | | |
| GO:0090171 | chondrocyte | 18 | 1 | 1 | 1.93E−05 | 1 | 1 |
| | morphogenesis | | | | | | |
| GO:0030001 | metal ion transport | 858 | 0.616825534 | 1 | 2.52E−05 | 1 | 1 |
| GO:0003422 | growth plate cartilage | 19 | 1 | 1 | 2.83E−05 | 1 | 1 |
| | morphogenesis | | | | | | |
| GO:0022008 | neurogenesis | 1555 | 0.665596251 | 1 | 3.83E−05 | 0.139859641 | 1 |
| GO:0007155 | cell adhesion | 1390 | 0.001076518 | 1 | 4.21E−05 | 0.598967854 | 1 |
| GO:0048699 | generation of neurons | 1459 | 0.650883021 | 1 | 4.35E−05 | 0.280105182 | 1 |
| GO:0022610 | biological adhesion | 1398 | 0.001270754 | 1 | 5.07E−05 | 0.445725934 | 1 |
| GO:0034762 | regulation of | 535 | 1 | 1 | 5.53E−05 | 1 | 1 |
| | transmembrane | | | | | | |
| | transport | | | | | | |
| GO:0055085 | transmembrane | 1514 | 1 | 1 | 6.85E−05 | 1 | 1 |
| | transport | | | | | | |
| GO:0030182 | neuron differentiation | 1317 | 0.768500111 | 1 | 7.84E−05 | 0.415235372 | 1 |
| GO:0003416 | endochondral bone | 43 | 1 | 1 | 8.11E−05 | 1 | 1 |
| | growth | | | | | | |
| GO:0003433 | chondrocyte | 23 | 1 | 1 | 0.000111249 | 1 | 1 |
| | development involved | | | | | | |
| | in endochondral bone | | | | | | |
| | morphogenesis | | | | | | |
| GO:0098868 | bone growth | 45 | 1 | 1 | 0.000118729 | 1 | 1 |
| GO:0032989 | cellular component | 1079 | 0.019925787 | 0.99915203 | 0.000144687 | 0.890858017 | 0.757703978 |
| | morphogenesis | | | | | | |
| GO:0003418 | growth plate cartilage | 24 | 1 | 1 | 0.000151713 | 1 | 1 |
| | chondrocyte | | | | | | |
| | differentiation | | | | | | |
| GO:0003417 | growth plate cartilage | 36 | 1 | 1 | 0.000217688 | 1 | 1 |
| | development | | | | | | |
| GO:0007268 | chemical synaptic | 690 | 0.480223653 | 1 | 0.000225474 | 0.324979313 | 1 |
| | transmission | | | | | | |
| GO:0098916 | anterograde trans- | 690 | 0.480223653 | 1 | 0.000225474 | 0.324979313 | 1 |
| | synaptic signaling | | | | | | |
| GO:0006814 | sodium ion transport | 215 | 1 | 1 | 0.00025748 | 1 | 1 |
| GO:0099537 | trans-synaptic | 698 | 0.517697406 | 1 | 0.000288644 | 0.347784599 | 1 |
| | signaling | | | | | | |
| GO:0099536 | synaptic signaling | 703 | 0.275739603 | 1 | 0.000337001 | 0.357514166 | 1 |
| GO:0009888 | tissue development | 1961 | 1 | 1 | 0.000413598 | 1 | 1 |
| GO:0050954 | sensory perception of | 161 | 1 | 1 | 0.000527109 | 1 | 1 |
| | mechanical stimulus | | | | | | |
| GO:0003413 | chondrocyte | 29 | 1 | 1 | 0.000545066 | 1 | 1 |
| | differentiation | | | | | | |
| | involved in | | | | | | |
| | endochondral bone | | | | | | |
| | morphogenesis | | | | | | |
| GO:0007605 | sensory perception of | 142 | 1 | 1 | 0.000581084 | 1 | 1 |
| | sound | | | | | | |
| GO:0032879 | regulation of | 2746 | 4.21E−05 | 1 | 0.000674069 | 1 | 1 |
| | localization | | | | | | |
| GO:0060350 | endochondral bone | 71 | 1 | 0.901231612 | 0.000675474 | 1 | 1 |
| | morphogenesis | | | | | | |
| GO:0098655 | cation | 827 | 1 | 1 | 0.00080515 | 1 | 1 |
| | transmembrane | | | | | | |
| | transport | | | | | | |
| GO:2001223 | negative regulation of | 12 | 1 | 1 | 0.00080515 | 1 | 1 |
| | neuron migration | | | | | | |
| GO:0000904 | cell morphogenesis | 712 | 0.890858017 | 0.965146807 | 0.001032945 | 0.616825534 | 0.410213141 |
| | involved in | | | | | | |
| | differentiation | | | | | | |
| GO:0002063 | chondrocyte | 45 | 1 | 1 | 0.001076518 | 1 | 1 |
| | development | | | | | | |
| GO:0048513 | animal organ | 3455 | 1 | 0.063098951 | 0.00108333 | 0.745168516 | 0.665596251 |
| | development | | | | | | |
| GO:0060349 | bone morphogenesis | 111 | 1 | 0.469843251 | 0.00108657 | 1 | 1 |
| GO:0060351 | cartilage development | 46 | 1 | 1 | 0.001251135 | 1 | 1 |
| | involved in | | | | | | |
| | endochondral bone | | | | | | |
| | morphogenesis | | | | | | |
| GO:0000902 | cell morphogenesis | 980 | 0.022949891 | 1 | 0.001401799 | 0.797071186 | 0.796314167 |
| GO:0007267 | cell-cell signaling | 1584 | 0.543286651 | 1 | 0.001964875 | 0.171334526 | 1 |
| GO:0015672 | monovalent inorganic | 526 | 1 | 1 | 0.002079582 | 1 | 1 |
| | cation transport | | | | | | |
| GO:0098662 | inorganic cation | 739 | 1 | 1 | 0.002079582 | 1 | 1 |
| | transmembrane | | | | | | |
| | transport | | | | | | |
| GO:0044703 | multi-organism | 1004 | 1 | 1 | 0.002354643 | 1 | 1 |
| | reproductive process | | | | | | |
| GO:0048666 | neuron development | 1071 | 0.745168516 | 1 | 0.002354643 | 0.489378401 | 1 |
| GO:0042391 | regulation of | 424 | 1 | 1 | 0.003216776 | 0.716801532 | 1 |
| | membrane potential | | | | | | |
| GO:0006936 | muscle contraction | 351 | 0.357514166 | 1 | 0.004649326 | 1 | 1 |
| GO:0048589 | developmental | 646 | 1 | 1 | 0.004771785 | 1 | 1 |
| | growth | | | | | | |
| GO:0051049 | regulation of | 1806 | 0.0059057 | 1 | 0.006500421 | 1 | 1 |
| | transport | | | | | | |
| GO:1904062 | regulation of cation | 314 | 1 | 1 | 0.008881305 | 1 | 1 |
| | transmembrane | | | | | | |
| | transport | | | | | | |
| GO:0048705 | skeletal system | 236 | 1 | 1 | 0.009182742 | 0.996446813 | 1 |
| | morphogenesis | | | | | | |
| GO:0032412 | regulation of ion | 238 | 1 | 1 | 0.009921143 | 1 | 1 |
| | transmembrane | | | | | | |
| | transporter activity | | | | | | |
| GO:0031175 | neuron projection | 941 | 0.594734994 | 1 | 0.010301477 | 0.317513419 | 1 |
| | development | | | | | | |
| GO:0007389 | pattern specification | 434 | 1 | 1 | 0.011108198 | 1 | 1 |
| | process | | | | | | |
| GO:0030198 | extracellular matrix | 348 | 1 | 1 | 0.011108198 | 1 | 1 |
| | organization | | | | | | |
| GO:0098656 | anion transmembrane | 268 | 1 | 1 | 0.011788736 | 1 | 1 |
| | transport | | | | | | |
| GO:0048812 | neuron projection | 621 | 1 | 1 | 0.012239952 | 0.507490051 | 0.717048855 |
| | morphogenesis | | | | | | |
| GO:0032990 | cell part | 655 | 1 | 1 | 0.01290706 | 0.646941518 | 0.802166171 |
| | morphogenesis | | | | | | |
| GO:0022898 | regulation of | 245 | 1 | 1 | 0.013282485 | 1 | 1 |
| | transmembrane | | | | | | |
| | transporter activity | | | | | | |
| GO:0007160 | cell-matrix adhesion | 222 | 0.005606843 | 1 | 0.014820961 | 1 | 1 |
| GO:0022414 | reproductive process | 1423 | 1 | 1 | 0.015397049 | 1 | 1 |
| GO:0072025 | distal convoluted | 5 | 1 | 1 | 0.015849029 | 1 | 1 |
| | tubule development | | | | | | |
| GO:0072221 | metanephric dista | 5 | 1 | 1 | 0.015849029 | 1 | 1 |
| | convoluted tubule | | | | | | |
| | development | | | | | | |
| GO:0000003 | reproduction | 1426 | 1 | 1 | 0.015996351 | 1 | 1 |
| GO:0002009 | morphogenesis of an | 479 | 1 | 1 | 0.016789679 | 1 | 1 |
| | epithelium | | | | | | |
| GO:0120039 | plasma membrane | 635 | 1 | 1 | 0.016801155 | 0.56362124 | 0.745168516 |
| | bounded cell | | | | | | |
| | projection | | | | | | |
| | morphogenesis | | | | | | |
| GO:0048858 | cell projection | 636 | 1 | 1 | 0.017059196 | 0.568448591 | 0.745168516 |
| | morphogenesis | | | | | | |
| GO:0017156 | calcium ion regulated | 153 | 1 | 0.996446813 | 0.017665674 | 1 | 1 |
| | exocytosis | | | | | | |
| GO:0098661 | inorganic anion | 110 | 1 | 1 | 0.019146167 | 1 | 1 |
| | transmembrane | | | | | | |
| | transport | | | | | | |
| GO:0007215 | glutamate receptor | 90 | 1 | 1 | 0.019925787 | 0.665596251 | 1 |
| | signaling pathway | | | | | | |
| GO:0035249 | synaptic transmission, | 90 | 1 | 1 | 0.019925787 | 1 | 1 |
| | glutamatergic | | | | | | |
| GO:0050877 | nervous system | 1406 | 1 | 1 | 0.019979972 | 1 | 1 |
| | process | | | | | | |
| GO:0003012 | muscle system | 456 | 0.371617002 | 1 | 0.020005602 | 1 | 1 |
| | process | | | | | | |
| GO:1901018 | positive regulation of | 24 | 1 | 1 | 0.020370955 | 1 | 1 |
| | potassium ion | | | | | | |
| | transmembrane | | | | | | |
| | transporter activity | | | | | | |
| GO:0060429 | epithelium | 1227 | 1 | 1 | 0.020590857 | 1 | 1 |
| | development | | | | | | |
| GO:0007417 | central nervous | 948 | 1 | 0.996446813 | 0.021561593 | 0.922242032 | 1 |
| | system development | | | | | | |
| GO:0051216 | cartilage development | 206 | 1 | 1 | 0.021561593 | 0.78080559 | 1 |
| GO:0019226 | transmission of nerve | 72 | 1 | 1 | 0.022053268 | 1 | 1 |
| | impulse | | | | | | |
| GO:0051179 | localization | 6555 | 4.55E−10 | 1 | 0.022789146 | 0.56362124 | 1 |
| GO:0032409 | regulation of | 260 | 1 | 1 | 0.022963874 | 1 | 1 |
| | transporter activity | | | | | | |
| GO:0060348 | bone development | 208 | 1 | 0.489378401 | 0.023314211 | 1 | 1 |
| GO:0030048 | actin filament-based | 136 | 1 | 1 | 0.023984508 | 1 | 1 |
| | movement | | | | | | |
| GO:0030049 | muscle filament | 39 | 1 | 1 | 0.024126389 | 1 | 1 |
| | sliding | | | | | | |
| GO:0033275 | actin-myosin filament | 39 | 1 | 1 | 0.024126389 | 1 | 1 |
| | sliding | | | | | | |
| GO:0070252 | actin-mediated cell | 114 | 1 | 1 | 0.024126389 | 1 | 1 |
| | contraction | | | | | | |
| GO:2001222 | regulation of neuron | 39 | 1 | 1 | 0.024126389 | 1 | 1 |
| | migration | | | | | | |
| GO:0061448 | connective tissue | 263 | 1 | 1 | 0.02536056 | 0.219616377 | 1 |
| | development | | | | | | |
| GO:2001257 | regulation of cation | 162 | 1 | 1 | 0.026700112 | 1 | 1 |
| | channel activity | | | | | | |
| GO:0007423 | sensory organ | 532 | 1 | 1 | 0.027749721 | 1 | 1 |
| | development | | | | | | |
| GO:0002062 | chondrocyte | 117 | 1 | 1 | 0.0280421 | 1 | 1 |
| | differentiation | | | | | | |
| GO:0044057 | regulation of system | 569 | 0.278182469 | 1 | 0.031028127 | 1 | 1 |
| | process | | | | | | |
| GO:0001501 | skeletal system | 506 | 1 | 0.823129385 | 0.031162918 | 1 | 0.996446813 |
| | development | | | | | | |
| GO:1903818 | positive regulation of | 15 | 1 | 1 | 0.031196003 | 1 | 1 |
| | voltage-gated | | | | | | |
| | potassium channel | | | | | | |
| | activity | | | | | | |
| GO:0098742 | cell-cell adhesion via | 270 | 1 | 1 | 0.03127218 | 1 | 1 |
| | plasma-membrane | | | | | | |
| | adhesion molecules | | | | | | |
| GO:0007269 | neurotransmitter | 167 | 1 | 1 | 0.032811717 | 1 | 1 |
| | secretion | | | | | | |
| GO:0099643 | signal release from | 167 | 1 | 1 | 0.032811717 | 1 | 1 |
| | synapse | | | | | | |
| GO:0050804 | modulation of | 418 | 0.844000933 | 1 | 0.034515558 | 0.102842968 | 1 |
| | chemical synaptic | | | | | | |
| | transmission | | | | | | |
| GO:0099177 | regulation of trans- | 419 | 0.857332482 | 1 | 0.035278512 | 0.10434846 | 1 |
| | synaptic signaling | | | | | | |
| GO:0035265 | organ growth | 195 | 1 | 1 | 0.037148224 | 1 | 1 |
| GO:0044706 | multi-multicellular | 221 | 1 | 1 | 0.037148224 | 1 | 1 |
| | organism process | | | | | | |
| GO:0021537 | telencephalon | 248 | 1 | 1 | 0.037418398 | 1 | 1 |
| | development | | | | | | |
| GO:0016079 | synaptic vesicle | 123 | 1 | 1 | 0.038417362 | 1 | 1 |
| | exocytosis | | | | | | |
| GO:0051960 | regulation of nervous | 883 | 0.107843848 | 1 | 0.039675644 | 0.335240873 | 1 |
| | system development | | | | | | |
| GO:0072235 | metanephric distal | 7 | 1 | 1 | 0.039896554 | 1 | 1 |
| | tubule development | | | | | | |
| GO:0120036 | plasma membrane | 1462 | 0.430289651 | 1 | 0.040423722 | 0.184216961 | 1 |
| | bounded cell | | | | | | |
| | projection | | | | | | |
| | organization | | | | | | |
| GO:0060537 | muscle tissue | 395 | 1 | 1 | 0.041122974 | 1 | 1 |
| | development | | | | | | |
| GO:0043062 | extracellular structure | 402 | 1 | 1 | 0.04968339 | 1 | 1 |
| | organization | | | | | | |
| GO:0006928 | movement of cell or | 2138 | 8.70E−05 | 1 | 0.052850381 | 0.108871927 | 1 |
| | subcellular | | | | | | |
| | component | | | | | | |
| GO:0030029 | actin filament-based | 732 | 0.028430549 | 1 | 0.055807618 | 1 | 1 |
| | process | | | | | | |
| GO:0065008 | regulation of | 3891 | 1.72E−06 | 1 | 0.092517809 | 0.598967854 | 1 |
| | biological quality | | | | | | |
| GO:0031589 | cell-substrate | 338 | 0.004206765 | 1 | 0.093681736 | 0.890858017 | 1 |
| | adhesion | | | | | | |
| GO:0051239 | regulation of | 3093 | 2.64E−05 | 0.748743161 | 0.123095655 | 0.575098872 | 1 |
| | multicellular | | | | | | |
| | organismal process | | | | | | |
| GO:2000026 | regulation of | 2064 | 0.030521352 | 0.966211545 | 0.211865167 | 0.24144412 | 1 |
| | multicellular | | | | | | |
| | organismal | | | | | | |
| | development | | | | | | |
| GO:0007154 | cell communication | 6522 | 1.26E−09 | 1 | 0.2315925 | 0.035576453 | 1 |
| GO:0048167 | regulation of synaptic | 173 | 0.756136312 | 1 | 0.247322371 | 0.025766984 | 1 |
| | plasticity | | | | | | |
| GO:0009612 | response to | 204 | 0.016589491 | 1 | 0.264413515 | 1 | 1 |
| | mechanical stimulus | | | | | | |
| GO:0048646 | anatomical structure | 1151 | 0.017059196 | 0.665596251 | 0.278694883 | 0.131591479 | 1 |
| | formation involved in | | | | | | |
| | morphogenesis | | | | | | |
| GO:0007166 | cell surface receptor | 2974 | 2.22E−07 | 0.664638292 | 0.280083899 | 0.024 | 1 |
| | signaling pathway | | | | | | |
| GO:0050793 | regulation of | 2607 | 0.00358275 | 0.489378401 | 0.299390486 | 0.136920059 | 1 |
| | developmental | | | | | | |
| | process | | | | | | |
| GO:0045944 | positive regulation of | 1181 | 1 | 0.028430549 | 0.358787418 | 1 | 1 |
| | transcription by RNA | | | | | | |
| | polymerase II | | | | | | |
| GO:0006937 | regulation of muscle | 164 | 0.004919038 | 1 | 0.405813243 | 1 | 1 |
| | contraction | | | | | | |
| GO:0006810 | transport | 5065 | 6.95E−11 | 1 | 0.455598256 | 1 | 1 |
| GO:0022603 | regulation of | 1098 | 0.027537974 | 0.80941255 | 0.48419276 | 0.376392302 | 1 |
| | anatomical structure | | | | | | |
| | morphogenesis | | | | | | |
| GO:0023052 | signaling | 6475 | 5.74E−10 | 1 | 0.489378401 | 0.028424743 | 1 |
| GO:0060284 | regulation of cell | 910 | 0.043655125 | 1 | 0.490789374 | 0.61494051 | 1 |
| | development | | | | | | |
| GO:0051234 | establishment of | 5179 | 1.10E−10 | 1 | 0.491060511 | 1 | 1 |
| | localization | | | | | | |
| GO:0051270 | regulation of cellular | 1052 | 0.001076518 | 1 | 0.604011244 | 1 | 1 |
| | component | | | | | | |
| | movement | | | | | | |
| GO:0031644 | regulation of | 127 | 1 | 1 | 0.649678872 | 0.017681195 | 1 |
| | neurological system | | | | | | |
| | process | | | | | | |
| GO:0051240 | positive regulation of | 1753 | 0.000807618 | 1 | 0.664849722 | 1 | 1 |
| | multicellular | | | | | | |
| | organismal process | | | | | | |
| GO:0001568 | blood vessel | 756 | 0.027692049 | 1 | 0.665596251 | 0.190308867 | 1 |
| | development | | | | | | |
| GO:0072358 | cardiovascular system | 799 | 0.039048021 | 1 | 0.670434263 | 0.08846971 | 1 |
| | development | | | | | | |
| GO:0048518 | positive regulation of | 6054 | 2.12E−09 | 0.078665529 | 0.745168516 | 0.670434263 | 1 |
| | biological process | | | | | | |
| GO:0010574 | regulation of vascular | 32 | 0.006085233 | 1 | 0.762819911 | 1 | 1 |
| | endothelial growth | | | | | | |
| | factor production | | | | | | |
| GO:0050896 | response to stimulus | 9106 | 1.30E−09 | 0.767683656 | 0.762982012 | 0.125702334 | 1 |
| GO:0001944 | vasculature | 790 | 0.032663645 | 1 | 0.778782489 | 0.263544992 | 1 |
| | development | | | | | | |
| GO:0043410 | positive regulation of | 562 | 0.000386618 | 1 | 0.794945764 | 1 | 1 |
| | MAPK cascade | | | | | | |
| GO:0010573 | vascular endothelial | 34 | 0.008756837 | 1 | 0.823129385 | 1 | 1 |
| | growth factor | | | | | | |
| | production | | | | | | |
| GO:0045595 | regulation of cell | 1788 | 0.004325196 | 1 | 0.877494832 | 0.996446813 | 1 |
| | differentiation | | | | | | |
| GO:1903523 | negative regulation of | 36 | 0.012075239 | 1 | 0.890858017 | 1 | 1 |
| | blood circulation | | | | | | |
| GO:0045893 | positive regulation of | 1511 | 1 | 0.00087738 | 0.918797783 | 1 | 1 |
| | transcription, DNA- | | | | | | |
| | templated | | | | | | |
| GO:1902680 | positive regulation of | 1597 | 1 | 0.00087738 | 0.922242032 | 1 | 1 |
| | RNA biosynthetic | | | | | | |
| | process | | | | | | |
| GO:1903508 | positive regulation of | 1596 | 1 | 0.00086646 | 0.922242032 | 1 | 1 |
| | nucleic acid- | | | | | | |
| | templated | | | | | | |
| | transcription | | | | | | |
| GO:0002223 | stimulatory C-type | 65 | 0.017009962 | 1 | 0.972458544 | 1 | 1 |
| | lectin receptor | | | | | | |
| | signaling pathway | | | | | | |
| GO:0010628 | positive regulation of | 1957 | 1 | 0.004755649 | 0.995169577 | 1 | 1 |
| | gene expression | | | | | | |
| GO:0040011 | locomotion | 1909 | 2.16E−05 | 1 | 0.995169577 | 0.474177846 | 1 |
| GO:0002220 | innate immune | 68 | 0.022623601 | 1 | 0.996446813 | 1 | 1 |
| | response activating | | | | | | |
| | cell surface receptor | | | | | | |
| | signaling pathway | | | | | | |
| GO:0010646 | regulation of cell | 3532 | 3.18E−07 | 0.996446813 | 0.996446813 | 0.007264557 | 1 |
| | communication | | | | | | |
| GO:0048514 | blood vessel | 677 | 0.026184522 | 1 | 0.996446813 | 0.161086801 | 1 |
| | morphogenesis | | | | | | |
| GO:0071495 | cellular response to | 1373 | 0.011622623 | 0.308597466 | 0.996446813 | 1 | 0.161929325 |
| | endogenous stimulus | | | | | | |
| GO:0000122 | negative regulation of | 835 | 1 | 4.26E−10 | 1 | 1 | 1 |
| | transcription by RNA | | | | | | |
| | polymerase II | | | | | | |
| GO:0000165 | MAPK cascade | 926 | 0.000542536 | 1 | 1 | 1 | 1 |
| GO:0000186 | activation of MAPKK | 52 | 0.000574544 | 1 | 1 | 1 | 1 |
| | activity | | | | | | |
| GO:0000271 | polysaccharide | 74 | 0.009838404 | 1 | 1 | 1 | 1 |
| | biosynthetic process | | | | | | |
| GO:0000272 | polysaccharide | 23 | 0.007542869 | 1 | 1 | 1 | 1 |
| | catabolic process | | | | | | |
| GO:0001525 | angiogenesis | 587 | 0.009359443 | 1 | 1 | 0.211865167 | 1 |
| GO:0001702 | gastrulation with | 28 | 1 | 1 | 1 | 1 | 0.007070407 |
| | mouth forming | | | | | | |
| | second | | | | | | |
| GO:0001774 | microglial cell | 33 | 0.007271618 | 1 | 1 | 1 | 1 |
| | activation | | | | | | |
| GO:0001775 | cell activation | 1407 | 9.24E−23 | 0.890858017 | 1 | 1 | 1 |
| GO:0001776 | leukocyte | 88 | 0.03127218 | 1 | 1 | 1 | 1 |
| | homeostasis | | | | | | |
| GO:0001780 | neutrophil | 16 | 0.012199993 | 1 | 1 | 1 | 1 |
| | homeostasis | | | | | | |
| GO:0001816 | cytokine production | 716 | 8.54E−07 | 0.243930235 | 1 | 1 | 1 |
| GO:0001817 | regulation of cytokine | 647 | 9.86E−06 | 0.457630758 | 1 | 1 | 1 |
| | production | | | | | | |
| GO:0001819 | positive regulation of | 422 | 0.000246192 | 1 | 1 | 1 | 1 |
| | cytokine production | | | | | | |
| GO:0001932 | regulation of protein | 1427 | 3.03E−07 | 1 | 1 | 1 | 1 |
| | phosphorylation | | | | | | |
| GO:0001934 | positive regulation of | 1008 | 1.75E−07 | 1 | 1 | 1 | 1 |
| | protein | | | | | | |
| | phosphorylation | | | | | | |
| GO:0002218 | activation of innate | 252 | 3.46E−05 | 1 | 1 | 1 | 1 |
| | immune response | | | | | | |
| GO:0002221 | pattern recognition | 179 | 0.004057705 | 1 | 1 | 1 | 1 |
| | receptor signaling | | | | | | |
| | pathway | | | | | | |
| GO:0002224 | toll-like receptor | 131 | 0.01844494 | 1 | 1 | 1 | 1 |
| | signaling pathway | | | | | | |
| GO:0002237 | response to molecule | 337 | 0.000205114 | 1 | 1 | 1 | 1 |
| | of bacterial origin | | | | | | |
| GO:0002244 | hematopoietic | 111 | 1 | 0.002200796 | 1 | 1 | 1 |
| | progenitor cell | | | | | | |
| | differentiation | | | | | | |
| GO:0002252 | immune effector | 1246 | 7.83E−19 | 1 | 1 | 1 | 1 |
| | process | | | | | | |
| GO:0002253 | activation of immune | 645 | 4.02E−06 | 1 | 1 | 1 | 1 |
| | response | | | | | | |
| GO:0002262 | myeloid cell | 146 | 0.041941486 | 0.025357726 | 1 | 1 | 1 |
| | homeostasis | | | | | | |
| GO:0002263 | cell activation | 701 | 2.15E−17 | 1 | 1 | 1 | 1 |
| | involved in immune | | | | | | |
| | response | | | | | | |
| GO:0002269 | leukocyte activation | 33 | 0.007271618 | 1 | 1 | 1 | 1 |
| | involved in | | | | | | |
| | inflammatory | | | | | | |
| | response | | | | | | |
| GO:0002274 | myeloid leukocyte | 638 | 9.90E−23 | 1 | 1 | 1 | 1 |
| | activation | | | | | | |
| GO:0002275 | myeloid cell activation | 540 | 3.02E−20 | 1 | 1 | 1 | 1 |
| | involved in immune | | | | | | |
| | response | | | | | | |
| GO:0002283 | neutrophil activation | 488 | 6.49E−20 | 1 | 1 | 1 | 1 |
| | involved in immune | | | | | | |
| | response | | | | | | |
| GO:0002366 | leukocyte activation | 697 | 1.62E−17 | 1 | 1 | 1 | 1 |
| | involved in immune | | | | | | |
| | response | | | | | | |
| GO:0002367 | cytokine production | 95 | 0.016140011 | 1 | 1 | 1 | 1 |
| | involved in immune | | | | | | |
| | response | | | | | | |
| GO:0002376 | immune system | 3074 | 1.22E−20 | 0.892839413 | 1 | 1 | 1 |
| | process | | | | | | |
| GO:0002429 | immune response- | 414 | 0.006508093 | 1 | 1 | 1 | 1 |
| | activating cell surface | | | | | | |
| | receptor signaling | | | | | | |
| | pathway | | | | | | |
| GO:0002443 | leukocyte mediated | 868 | 9.50E−17 | 1 | 1 | 1 | 1 |
| | immunity | | | | | | |
| GO:0002444 | myeloid leukocyte | 548 | 5.49E−20 | 1 | 1 | 1 | 1 |
| | mediated immunity | | | | | | |
| GO:0002446 | neutrophil mediated | 499 | 5.03E−20 | 1 | 1 | 1 | 1 |
| | immunity | | | | | | |
| GO:0002520 | immune system | 912 | 0.009644361 | 0.000500171 | 1 | 1 | 0.405813243 |
| | development | | | | | | |
| GO:0002521 | leukocyte | 501 | 0.212378102 | 0.017749668 | 1 | 1 | 1 |
| | differentiation | | | | | | |
| GO:0002576 | platelet degranulation | 128 | 1.37E−05 | 1 | 1 | 1 | 1 |
| GO:0002682 | regulation of immune | 1545 | 1.30E−09 | 1 | 1 | 1 | 1 |
| | system process | | | | | | |
| GO:0002684 | positive regulation of | 1098 | 1.33E−08 | 1 | 1 | 1 | 1 |
| | immune system | | | | | | |
| | process | | | | | | |
| GO:0002685 | regulation of | 180 | 0.013038389 | 1 | 1 | 1 | 1 |
| | leukocyte migration | | | | | | |
| GO:0002687 | positive regulation of | 121 | 0.009448392 | 0.931021722 | 1 | 1 | 1 |
| | leukocyte migration | | | | | | |
| GO:0002688 | regulation of | 109 | 0.013452901 | 1 | 1 | 1 | 1 |
| | leukocyte chemotaxis | | | | | | |
| GO:0002697 | regulation of immune | 439 | 0.029881063 | 1 | 1 | 1 | 1 |
| | effector process | | | | | | |
| GO:0002704 | negative regulation of | 45 | 0.037418398 | 1 | 1 | 1 | 1 |
| | leukocyte mediated | | | | | | |
| | immunity | | | | | | |
| GO:0002718 | regulation of cytokine | 77 | 0.046629493 | 0.997059037 | 1 | 1 | 1 |
| | production involved in | | | | | | |
| | immune response | | | | | | |
| GO:0002755 | MyD88-dependent | 36 | 0.001691861 | 1 | 1 | 1 | 1 |
| | toll-like receptor | | | | | | |
| | signaling pathway | | | | | | |
| GO:0002757 | immune response- | 568 | 1.34E−05 | 1 | 1 | 1 | 1 |
| | activating signal | | | | | | |
| | transduction | | | | | | |
| GO:0002758 | innate immune | 233 | 0.000110092 | 1 | 1 | 1 | 1 |
| | response-activating | | | | | | |
| | signal transduction | | | | | | |
| GO:0002764 | immune response- | 599 | 8.40E−06 | 1 | 1 | 1 | 1 |
| | regulating signaling | | | | | | |
| | pathway | | | | | | |
| GO:0002768 | immune response- | 445 | 0.003916178 | 1 | 1 | 1 | 1 |
| | regulating cell surface | | | | | | |
| | receptor signaling | | | | | | |
| | pathway | | | | | | |
| GO:0002790 | peptide secretion | 582 | 0.028669624 | 1 | 1 | 1 | 1 |
| GO:0005976 | polysaccharide | 109 | 0.003588731 | 1 | 1 | 1 | 1 |
| | metabolic process | | | | | | |
| GO:0006022 | aminoglycan | 167 | 0.01778747 | 0.870337648 | 1 | 1 | 1 |
| | metabolic process | | | | | | |
| GO:0006139 | nucleobase-containing | 6264 | 1 | 0.002380221 | 1 | 1 | 1 |
| | compound metabolic | | | | | | |
| | process | | | | | | |
| GO:0006351 | transcription, DNA- | 3600 | 1 | 5.47E−06 | 1 | 1 | 1 |
| | templated | | | | | | |
| GO:0006355 | regulation of | 3445 | 1 | 3.35E−06 | 1 | 1 | 1 |
| | transcription, DNA- | | | | | | |
| | templated | | | | | | |
| GO:0006357 | regulation of | 2632 | 1 | 6.40E−06 | 1 | 1 | 1 |
| | transcription by RNA | | | | | | |
| | polymerase II | | | | | | |
| GO:0006366 | transcription by RNA | 2768 | 1 | 8.50E−06 | 1 | 1 | 1 |
| | polymerase II | | | | | | |
| GO:0006464 | cellular protein | 4066 | 1.66E−05 | 0.93115264 | 1 | 1 | 1 |
| | modification process | | | | | | |
| GO:0006468 | protein | 1910 | 1.14E−06 | 1 | 1 | 0.635655637 | 1 |
| | phosphorylation | | | | | | |
| GO:0006629 | lipid metabolic | 1415 | 0.034515558 | 1 | 1 | 1 | 1 |
| | process | | | | | | |
| GO:0006725 | cellular aromatic | 6467 | 1 | 0.008831261 | 1 | 1 | 1 |
| | compound metabolic | | | | | | |
| | process | | | | | | |
| GO:0006793 | phosphorus metabolic | 3236 | 1.32E−06 | 1 | 1 | 1 | 1 |
| | process | | | | | | |
| GO:0006796 | phosphate-containing | 3209 | 7.74E−07 | 1 | 1 | 1 | 1 |
| | compound metabolic | | | | | | |
| | process | | | | | | |
| GO:0006826 | iron ion transport | 61 | 0.044765835 | 1 | 1 | 1 | 1 |
| GO:0006886 | intracellular protein | 1126 | 0.006155434 | 1 | 1 | 1 | 1 |
| | transport | | | | | | |
| GO:0006887 | exocytosis | 897 | 1.08E−23 | 1 | 1 | 1 | 1 |
| GO:0006897 | endocytosis | 783 | 2.44E−05 | 1 | 1 | 1 | 1 |
| GO:0006909 | phagocytosis | 342 | 0.011958563 | 1 | 1 | 1 | 1 |
| GO:0006914 | autophagy | 466 | 0.003701303 | 1 | 1 | 1 | 1 |
| GO:0006915 | apoptotic process | 1966 | 0.001910279 | 1 | 1 | 1 | 1 |
| GO:0006935 | chemotaxis | 619 | 0.01174754 | 1 | 1 | 1 | 1 |
| GO:0006940 | regulation of smooth | 62 | 0.049426883 | 1 | 1 | 1 | 1 |
| | muscle contraction | | | | | | |
| GO:0006950 | response to stress | 3908 | 5.50E−12 | 1 | 1 | 1 | 1 |
| GO:0006952 | defense response | 1658 | 5.06E−10 | 1 | 1 | 1 | 1 |
| GO:0006954 | inflammatory | 780 | 4.60E−06 | 1 | 1 | 1 | 1 |
| | response | | | | | | |
| GO:0006955 | immune response | 2183 | 1.32E−19 | 1 | 1 | 1 | 1 |
| GO:0007159 | leukocyte cell-cell | 327 | 0.002534667 | 1 | 1 | 1 | 1 |
| | adhesion | | | | | | |
| GO:0007165 | signal transduction | 6031 | 1.51E−09 | 1 | 1 | 0.0521109 | 1 |
| GO:0007169 | transmembrane | 694 | 0.006256544 | 1 | 1 | 0.996446813 | 1 |
| | receptor protein | | | | | | |
| | tyrosine kinase | | | | | | |
| | signaling pathway | | | | | | |
| GO:0007249 | I-kappaB kinase/NF- | 258 | 0.00466681 | 1 | 1 | 1 | 1 |
| | kappaB signaling | | | | | | |
| GO:0007264 | small GTPase | 547 | 0.00566515 | 1 | 1 | 1 | 1 |
| | mediated signal | | | | | | |
| | transduction | | | | | | |
| GO:0007369 | gastrulation | 181 | 1 | 1 | 1 | 1 | 0.031028127 |
| GO:0007568 | Aging | 311 | 1 | 0.019378819 | 1 | 1 | 1 |
| GO:0007596 | blood coagulation | 334 | 1.70E−08 | 1 | 1 | 1 | 1 |
| GO:0007599 | hemostasis | 339 | 6.82E−09 | 0.901976979 | 1 | 1 | 1 |
| GO:0008104 | protein localization | 2693 | 2.67E−05 | 1 | 1 | 1 | 1 |
| GO:0008152 | metabolic process | 12421 | 1 | 0.031337146 | 1 | 1 | 1 |
| GO:0008154 | actin polymerization | 206 | 0.043279439 | 1 | 1 | 1 | 1 |
| | or depolymerization | | | | | | |
| GO:0008219 | cell death | 2232 | 0.003677466 | 1 | 1 | 1 | 1 |
| GO:0009056 | catabolic process | 2490 | 1.66E−09 | 1 | 1 | 1 | 1 |
| GO:0009057 | macromolecule | 1346 | 9.85E−07 | 1 | 1 | 1 | 1 |
| | catabolic process | | | | | | |
| GO:0009058 | biosynthetic process | 6176 | 1 | 4.50E−06 | 1 | 1 | 1 |
| GO:0009059 | macromolecule | 4998 | 1 | 1.65E−06 | 1 | 1 | 1 |
| | biosynthetic process | | | | | | |
| GO:0009251 | glucan catabolic | 19 | 0.024975729 | 1 | 1 | 1 | 1 |
| | process | | | | | | |
| GO:0009267 | cellular response to | 146 | 0.015656927 | 1 | 1 | 1 | 1 |
| | starvation | | | | | | |
| GO:0009306 | protein secretion | 550 | 0.024183556 | 1 | 1 | 1 | 1 |
| GO:0009605 | response to external | 2410 | 1.65E−09 | 1 | 1 | 1 | 1 |
| | stimulus | | | | | | |
| GO:0009607 | response to biotic | 1014 | 3.97E−08 | 1 | 1 | 1 | 1 |
| | stimulus | | | | | | |
| GO:0009611 | response to wounding | 655 | 9.77E−07 | 1 | 1 | 0.876970475 | 1 |
| GO:0009615 | response to virus | 315 | 0.000527109 | 1 | 1 | 1 | 1 |
| GO:0009617 | response to bacterium | 683 | 0.000497498 | 1 | 1 | 1 | 1 |
| GO:0009636 | response to toxic | 513 | 0.03174739 | 1 | 1 | 1 | 1 |
| | substance | | | | | | |
| GO:0009889 | regulation of | 4259 | 1 | 7.74E−06 | 1 | 1 | 1 |
| | biosynthetic process | | | | | | |
| GO:0009890 | negative regulation of | 1625 | 1 | 1.64E−06 | 1 | 1 | 1 |
| | biosynthetic process | | | | | | |
| GO:0009891 | positive regulation of | 1978 | 1 | 0.001496726 | 1 | 1 | 1 |
| | biosynthetic process | | | | | | |
| GO:0009892 | negative regulation of | 3395 | 1 | 0.026947894 | 1 | 1 | 1 |
| | metabolic process | | | | | | |
| GO:0009893 | positive regulation of | 3558 | 2.54E−05 | 0.065080058 | 1 | 0.796254883 | 1 |
| | metabolic process | | | | | | |
| GO:0009894 | regulation of catabolic | 875 | 5.35E−06 | 0.995169577 | 1 | 1 | 1 |
| | process | | | | | | |
| GO:0009896 | positive regulation of | 407 | 0.002076463 | 1 | 1 | 1 | 1 |
| | catabolic process | | | | | | |
| GO:0009966 | regulation of signal | 3183 | 8.98E−08 | 0.668982235 | 1 | 0.022834087 | 1 |
| | transduction | | | | | | |
| GO:0009967 | positive regulation of | 1606 | 4.08E−09 | 1 | 1 | 1 | 1 |
| | signal transduction | | | | | | |
| GO:0009987 | cellular process | 16779 | 0.004881927 | 0.604011244 | 1 | 1 | 1 |
| GO:0009991 | response to | 513 | 0.03174739 | 0.520071834 | 1 | 1 | 1 |
| | extracellular stimulus | | | | | | |
| GO:0010033 | response to organic | 3172 | 1.22E−08 | 0.197199227 | 1 | 0.967833783 | 1 |
| | substance | | | | | | |
| GO:0010243 | response to | 950 | 5.79E−05 | 1 | 1 | 1 | 1 |
| | organonitrogen | | | | | | |
| | compound | | | | | | |
| GO:0010468 | regulation of gene | 4989 | 1 | 0.017606755 | 1 | 1 | 1 |
| | expression | | | | | | |
| GO:0010543 | regulation of platelet | 30 | 0.027236556 | 1 | 1 | 1 | 1 |
| | activation | | | | | | |
| GO:0010556 | regulation of | 4041 | 1 | 3.35E−06 | 1 | 1 | 1 |
| | macromolecule | | | | | | |
| | biosynthetic process | | | | | | |
| GO:0010557 | positive regulation of | 1844 | 1 | 0.000674069 | 1 | 1 | 1 |
| | macromolecule | | | | | | |
| | biosynthetic process | | | | | | |
| GO:0010558 | negative regulation of | 1536 | 1 | 3.22E−07 | 1 | 1 | 1 |
| | macromolecule | | | | | | |
| | biosynthetic process | | | | | | |
| GO:0010562 | positive regulation of | 1124 | 3.91E−07 | 1 | 1 | 1 | 1 |
| | phosphorus metabolic | | | | | | |
| | process | | | | | | |
| GO:0010604 | positive regulation of | 3299 | 1.05E−05 | 0.09101847 | 1 | 0.665596251 | 1 |
| | macromolecule | | | | | | |
| | metabolic process | | | | | | |
| GO:0010605 | negative regulation of | 3164 | 1 | 0.009382245 | 1 | 1 | 1 |
| | macromolecule | | | | | | |
| | metabolic process | | | | | | |
| GO:0010629 | negative regulation of | 2326 | 1 | 0.011891493 | 1 | 1 | 1 |
| | gene expression | | | | | | |
| GO:0010638 | positive regulation of | 602 | 0.014166664 | 1 | 1 | 1 | 1 |
| | organelle organization | | | | | | |
| GO:0010647 | positive regulation of | 1768 | 1.33E−08 | 1 | 1 | 0.957657142 | 1 |
| | cell communication | | | | | | |
| GO:0010720 | positive regulation of | 529 | 0.026184522 | 1 | 1 | 1 | 1 |
| | cell development | | | | | | |
| GO:0010821 | regulation of | 176 | 0.00334288 | 1 | 1 | 1 | 1 |
| | mitochondrion | | | | | | |
| | organization | | | | | | |
| GO:0010822 | positive regulation of | 112 | 0.049884217 | 1 | 1 | 1 | 1 |
| | mitochondrion | | | | | | |
| | organization | | | | | | |
| GO:0010941 | regulation of cell | 1707 | 0.038843865 | 1 | 1 | 1 | 1 |
| | death | | | | | | |
| GO:0010942 | positive regulation of | 689 | 0.001270746 | 1 | 1 | 1 | 1 |
| | cell death | | | | | | |
| GO:0010950 | positive regulation of | 171 | 0.022053268 | 1 | 1 | 0.933555354 | 1 |
| | endopeptidase activity | | | | | | |
| GO:0010952 | positive regulation of | 189 | 0.007469011 | 1 | 1 | 1 | 1 |
| | peptidase activity | | | | | | |
| GO:0012501 | programmed cell | 2099 | 0.002691077 | 1 | 1 | 1 | 1 |
| | death | | | | | | |
| GO:0015031 | protein transport | 1974 | 1.57E−05 | 1 | 1 | 1 | 1 |
| GO:0015669 | gas transport | 19 | 1 | 1 | 1 | 1 | 0.035229966 |
| GO:0015833 | peptide transport | 2009 | 3.25E−05 | 1 | 1 | 1 | 1 |
| GO:0016043 | cellular component | 6334 | 0.034533479 | 0.905458796 | 1 | 0.489378401 | 0.102751857 |
| | organization | | | | | | |
| GO:0016045 | detection of | 12 | 0.037418398 | 1 | 1 | 1 | 1 |
| | bacterium | | | | | | |
| GO:0016050 | vesicle organization | 314 | 0.003665448 | 1 | 1 | 1 | 1 |
| GO:0016070 | RNA metabolic | 5106 | 1 | 0.001250472 | 1 | 1 | 1 |
| | process | | | | | | |
| GO:0016192 | vesicle-mediated | 2058 | 7.47E−19 | 1 | 1 | 1 | 1 |
| | transport | | | | | | |
| GO:0016310 | phosphorylation | 2303 | 9.46E−06 | 1 | 1 | 1 | 1 |
| GO:0016477 | cell migration | 1537 | 1.08E−05 | 1 | 1 | 0.442735373 | 1 |
| GO:0018130 | heterocycle | 4233 | 1 | 8.40E−06 | 1 | 1 | 1 |
| | biosynthetic process | | | | | | |
| GO:0019219 | regulation of | 4013 | 1 | 5.63E−06 | 1 | 1 | 1 |
| | nucleobase-containing | | | | | | |
| | compound metabolic | | | | | | |
| | process | | | | | | |
| GO:0019220 | regulation of | 1736 | 7.18E−07 | 1 | 1 | 1 | 1 |
| | phosphate metabolic | | | | | | |
| | process | | | | | | |
| GO:0019221 | cytokine-mediated | 728 | 2.37E−07 | 1 | 1 | 1 | 1 |
| | signaling pathway | | | | | | |
| GO:0019222 | regulation of | 7129 | 0.108871927 | 0.001496726 | 1 | 1 | 1 |
| | metabolic process | | | | | | |
| GO:0019438 | aromatic compound | 4242 | 1 | 9.45E−06 | 1 | 1 | 1 |
| | biosynthetic process | | | | | | |
| GO:0019538 | protein metabolic | 5855 | 0.000885922 | 1 | 1 | 0.93226801 | 1 |
| | process | | | | | | |
| GO:0022604 | regulation of cell | 474 | 0.040355298 | 1 | 1 | 1 | 1 |
| | morphogenesis | | | | | | |
| GO:0023014 | signal transduction by | 944 | 0.000451981 | 1 | 1 | 1 | 1 |
| | protein | | | | | | |
| | phosphorylation | | | | | | |
| GO:0023051 | regulation of signaling | 3567 | 1.20E−07 | 0.918797783 | 1 | 0.00944828 | 1 |
| GO:0023056 | positive regulation of | 1775 | 3.71E−09 | 1 | 1 | 0.984890815 | 1 |
| | signaling | | | | | | |
| GO:0030097 | hemopoiesis | 819 | 0.034690044 | 0.000140896 | 1 | 1 | 0.686175641 |
| GO:0030098 | lymphocyte | 344 | 1 | 0.026584093 | 1 | 1 | 1 |
| | differentiation | | | | | | |
| GO:0030099 | myeloid cell | 405 | 0.04295607 | 0.134152259 | 1 | 1 | 1 |
| | differentiation | | | | | | |
| GO:0030100 | regulation of | 260 | 0.013452901 | 1 | 1 | 1 | 1 |
| | endocytosis | | | | | | |
| GO:0030155 | regulation of cell | 668 | 0.002919532 | 1 | 1 | 1 | 1 |
| | adhesion | | | | | | |
| GO:0030163 | protein catabolic | 900 | 0.002021862 | 1 | 1 | 1 | 1 |
| | process | | | | | | |
| GO:0030168 | platelet activation | 152 | 4.20E−07 | 0.996446813 | 1 | 1 | 1 |
| GO:0030193 | regulation of blood | 77 | 0.002919532 | 1 | 1 | 1 | 1 |
| | coagulation | | | | | | |
| GO:0030213 | hyaluronan | 13 | 0.049467321 | 0.93226801 | 1 | 1 | 1 |
| | biosynthetic process | | | | | | |
| GO:0030218 | erythrocyte | 111 | 0.897478797 | 0.002200796 | 1 | 1 | 0.879865755 |
| | differentiation | | | | | | |
| GO:0030220 | platelet formation | 20 | 1 | 0.023984508 | 1 | 1 | 1 |
| GO:0030334 | regulation of cell | 910 | 0.001353029 | 1 | 1 | 1 | 1 |
| | migration | | | | | | |
| GO:0030335 | positive regulation of | 519 | 4.00E−06 | 1 | 1 | 1 | 1 |
| | cell migration | | | | | | |
| GO:0030593 | neutrophil chemotaxis | 101 | 0.007127455 | 1 | 1 | 1 | 1 |
| GO:0030595 | leukocyte chemotaxis | 217 | 0.001379065 | 1 | 1 | 1 | 1 |
| GO:0030851 | granulocyte | 32 | 1 | 0.027161313 | 1 | 1 | 1 |
| | differentiation | | | | | | |
| GO:0031098 | stress-activated | 313 | 0.008881305 | 1 | 1 | 1 | 1 |
| | protein kinase | | | | | | |
| | signaling cascade | | | | | | |
| GO:0031323 | regulation of cellular | 6127 | 0.000154745 | 5.63E−06 | 1 | 1 | 1 |
| | metabolic process | | | | | | |
| GO:0031324 | negative regulation of | 2571 | 0.231941565 | 3.05E−05 | 1 | 1 | 1 |
| | cellular metabolic | | | | | | |
| | process | | | | | | |
| GO:0031325 | positive regulation of | 3260 | 1.46E−05 | 0.026184522 | 1 | 1 | 1 |
| | cellular metabolic | | | | | | |
| | process | | | | | | |
| GO:0031326 | regulation of cellular | 4184 | 1 | 6.54E−06 | 1 | 1 | 1 |
| | biosynthetic process | | | | | | |
| GO:0031327 | negative regulation of | 1601 | 1 | 1.75E−06 | 1 | 1 | 1 |
| | cellular biosynthetic | | | | | | |
| | process | | | | | | |
| GO:0031328 | positive regulation of | 1943 | 1 | 0.000816714 | 1 | 1 | 1 |
| | cellular biosynthetic | | | | | | |
| | process | | | | | | |
| GO:0031329 | regulation of cellular | 765 | 6.52E−05 | 0.896075185 | 1 | 1 | 1 |
| | catabolic process | | | | | | |
| GO:0031331 | positive regulation of | 347 | 0.006173714 | 0.989561623 | 1 | 1 | 1 |
| | cellular catabolic | | | | | | |
| | process | | | | | | |
| GO:0031334 | positive regulation of | 259 | 0.029415629 | 0.973367399 | 1 | 1 | 1 |
| | protein complex | | | | | | |
| | assembly | | | | | | |
| GO:0031347 | regulation of defense | 765 | 5.92E−06 | 1 | 1 | 1 | 1 |
| | response | | | | | | |
| GO:0031349 | positive regulation of | 427 | 5.88E−06 | 1 | 1 | 1 | 1 |
| | defense response | | | | | | |
| GO:0031399 | regulation of protein | 1821 | 2.37E−07 | 1 | 1 | 1 | 1 |
| | modification process | | | | | | |
| GO:0031401 | positive regulation of | 1220 | 8.58E−07 | 1 | 1 | 1 | 1 |
| | protein modification | | | | | | |
| | process | | | | | | |
| GO:0031646 | positive regulation of | 58 | 1 | 1 | 1 | 0.009678209 | 1 |
| | neurological system | | | | | | |
| | process | | | | | | |
| GO:0031663 | lipopolysaccharide- | 56 | 0.006197688 | 1 | 1 | 1 | 1 |
| | mediated signaling | | | | | | |
| | pathway | | | | | | |
| GO:0031667 | response to nutrient | 481 | 0.026184522 | 0.675996209 | 1 | 1 | 1 |
| | levels | | | | | | |
| | cellular response to | 258 | 0.062580021 | 0.032811717 | 1 | 1 | 1 |
| GO:0031668 | extracellular stimulus | | | | | | |
| GO:0031669 | cellular response to | 228 | 0.045842089 | 0.056922458 | 1 | 1 | 1 |
| | nutrient levels | | | | | | |
| GO:0032092 | positive regulation of | 94 | 0.048234344 | 1 | 1 | 1 | 1 |
| | protein binding | | | | | | |
| GO:0032101 | regulation of response | 840 | 0.001513988 | 1 | 1 | 1 | 1 |
| | to external stimulus | | | | | | |
| GO:0032103 | positive regulation of | 297 | 0.024975729 | 1 | 1 | 1 | 1 |
| | response to external | | | | | | |
| | stimulus | | | | | | |
| GO:0032147 | activation of protein | 323 | 3.00E−05 | 1 | 1 | 1 | 1 |
| | kinase activity | | | | | | |
| GO:0032268 | regulation of cellular | 2616 | 1.27E−07 | 1 | 1 | 1 | 1 |
| | protein metabolic | | | | | | |
| | process | | | | | | |
| GO:0032269 | negative regulation of | 1083 | 0.033725236 | 1 | 1 | 1 | 1 |
| | cellular protein | | | | | | |
| | metabolic process | | | | | | |
| GO:0032270 | positive regulation of | 1584 | 8.98E−08 | 1 | 1 | 1 | 1 |
| | cellular protein | | | | | | |
| | metabolic process | | | | | | |
| GO:0032496 | response to | 324 | 0.002200796 | 1 | 1 | 1 | 1 |
| | lipopolysaccharide | | | | | | |
| GO:0032611 | interleukin-1 beta | 73 | 0.033780469 | 1 | 1 | 1 | 1 |
| | production | | | | | | |
| GO:0032612 | interleukin-1 | 87 | 0.029127228 | 1 | 1 | 1 | 1 |
| | production | | | | | | |
| GO:0032637 | interleukin-8 | 73 | 0.001845049 | 1 | 1 | 1 | 1 |
| | production | | | | | | |
| GO:0032640 | tumor necrosis factor | 129 | 0.046405569 | 1 | 1 | 1 | 1 |
| | production | | | | | | |
| GO:0032677 | regulation of | 66 | 0.000765005 | 1 | 1 | 1 | 1 |
| | interleukin-8 | | | | | | |
| | production | | | | | | |
| GO:0032680 | regulation of tumor | 127 | 0.041122974 | 1 | 1 | 1 | 1 |
| | necrosis factor | | | | | | |
| | production | | | | | | |
| GO:0032757 | positive regulation of | 46 | 0.000194198 | 1 | 1 | 1 | 1 |
| | interleukin-8 | | | | | | |
| | production | | | | | | |
| GO:0032760 | positive regulation of | 73 | 0.033780469 | 1 | 1 | 1 | 1 |
| | tumor necrosis factor | | | | | | |
| | production | | | | | | |
| GO:0032774 | RNA biosynthetic | 3659 | 1 | 5.18E−06 | 1 | 1 | 1 |
| | process | | | | | | |
| GO:0032880 | regulation of protein | 963 | 9.45E−06 | 1 | 1 | 1 | 1 |
| | localization | | | | | | |
| GO:0032940 | secretion by cell | 1478 | 1.08E−23 | 1 | 1 | 1 | 1 |
| GO:0033036 | macromolecule | 3037 | 2.71E−05 | 1 | 1 | 1 | 1 |
| | localization | | | | | | |
| GO:0033043 | regulation of | 1237 | 0.018565448 | 0.869543798 | 1 | 1 | 1 |
| | organelle organization | | | | | | |
| GO:0033554 | cellular response to | 1928 | 0.000334829 | 1 | 1 | 1 | 1 |
| | stress | | | | | | |
| GO:0033674 | positive regulation of | 552 | 1.79E−09 | 1 | 1 | 1 | 1 |
| | kinase activity | | | | | | |
| GO:0034097 | response to cytokine | 1114 | 1.30E−07 | 1 | 1 | 1 | 1 |
| GO:0034101 | erythrocyte | 120 | 1 | 0.004673466 | 1 | 1 | 0.940589364 |
| | homeostasis | | | | | | |
| GO:0034109 | homotypic cell-cell | 76 | 0.04283723 | 0.996446813 | 1 | 1 | 1 |
| | adhesion | | | | | | |
| GO:0034123 | positive regulation of | 22 | 0.043674424 | 1 | 1 | 1 | 1 |
| | toll-like receptor | | | | | | |
| | signaling pathway | | | | | | |
| GO:0034134 | toll-like receptor 2 | 16 | 0.000949328 | 1 | 1 | 1 | 1 |
| | signaling pathway | | | | | | |
| GO:0034135 | regulation of toll-like | 11 | 0.027946094 | 1 | 1 | 1 | 1 |
| | receptor 2 signaling | | | | | | |
| | pathway | | | | | | |
| GO:0034198 | cellular response to | 45 | 0.037418398 | 1 | 1 | 1 | 1 |
| | amino acid starvation | | | | | | |
| GO:0034504 | protein localization to | 269 | 0.042659462 | 1 | 1 | 1 | 1 |
| | nucleus | | | | | | |
| GO:0034613 | cellular protein | 1830 | 0.000731018 | 1 | 1 | 1 | 1 |
| | localization | | | | | | |
| GO:0034641 | cellular nitrogen | 6998 | 1 | 0.006520896 | 1 | 1 | 1 |
| | compound metabolic | | | | | | |
| | process | | | | | | |
| GO:0034645 | cellular | 4853 | 1 | 8.55E−06 | 1 | 1 | 1 |
| | macromolecule | | | | | | |
| | biosynthetic process | | | | | | |
| GO:0034654 | nucleobase-containing | 4171 | 1 | 5.52E−06 | 1 | 1 | 1 |
| | compound | | | | | | |
| | biosynthetic process | | | | | | |
| GO:0034976 | response to | 264 | 0.015996351 | 1 | 1 | 1 | 1 |
| | endoplasmic | | | | | | |
| | reticulum stress | | | | | | |
| GO:0035556 | intracellular signal | 2817 | 1.30E−12 | 1 | 1 | 0.13056033 | 1 |
| | transduction | | | | | | |
| GO:0036211 | protein modification | 4066 | 1.66E−05 | 0.93115264 | 1 | 1 | 1 |
| | process | | | | | | |
| GO:0036230 | granulocyte activation | 505 | 4.83E−21 | 1 | 1 | 1 | 1 |
| GO:0036344 | platelet | 21 | 1 | 0.028400502 | 1 | 1 | 1 |
| | morphogenesis | | | | | | |
| GO:0038093 | Fc receptor signaling | 187 | 0.000173731 | 1 | 1 | 1 | 1 |
| | pathway | | | | | | |
| GO:0038094 | Fc-gamma receptor | 138 | 0.02750321 | 1 | 1 | 1 | 1 |
| | signaling pathway | | | | | | |
| GO:0040012 | regulation of | 1042 | 0.000435941 | 1 | 1 | 1 | 1 |
| | locomotion | | | | | | |
| GO:0040017 | positive regulation of | 570 | 6.01E−06 | 1 | 1 | 1 | 1 |
| | locomotion | | | | | | |
| GO:0042035 | regulation of cytokine | 94 | 0.048234344 | 0.24587016 | 1 | 1 | 1 |
| | biosynthetic process | | | | | | |
| GO:0042060 | wound healing | 543 | 2.91E−08 | 1 | 1 | 1 | 1 |
| GO:0042108 | positive regulation of | 57 | 0.031006288 | 1 | 1 | 1 | 1 |
| | cytokine biosynthetic | | | | | | |
| | process | | | | | | |
| GO:0042110 | T cell activation | 451 | 0.000878164 | 1 | 1 | 1 | 1 |
| GO:0042119 | neutrophil activation | 498 | 1.09E−20 | 1 | 1 | 1 | 1 |
| GO:0042221 | response to chemical | 4641 | 6.82E−06 | 0.969666228 | 1 | 1 | 1 |
| GO:0042325 | regulation of | 1536 | 7.74E−07 | 1 | 1 | 1 | 1 |
| | phosphorylation | | | | | | |
| GO:0042327 | positive regulation of | 1053 | 7.48E−08 | 1 | 1 | 1 | 1 |
| | phosphorylation | | | | | | |
| GO:0042330 | Taxis | 621 | 0.012287565 | 1 | 1 | 1 | 1 |
| GO:0042494 | detection of bacterial | 4 | 0.016140011 | 1 | 1 | 1 | 1 |
| | lipoprotein | | | | | | |
| GO:0042592 | homeostatic process | 1867 | 0.022963874 | 1 | 1 | 0.996446813 | 0.345762208 |
| GO:0042886 | amide transport | 2037 | 3.30E−05 | 1 | 1 | 1 | 1 |
| GO:0042981 | regulation of | 1572 | 0.029061136 | 1 | 1 | 1 | 1 |
| | apoptotic process | | | | | | |
| GO:0043065 | positive regulation of | 632 | 0.001982857 | 1 | 1 | 1 | 1 |
| | apoptotic process | | | | | | |
| GO:0043067 | regulation of | 1586 | 0.024126389 | 1 | 1 | 1 | 1 |
| | programmed cell | | | | | | |
| | death | | | | | | |
| GO:0043068 | positive regulation of | 636 | 0.001058108 | 1 | 1 | 1 | 1 |
| | programmed cell | | | | | | |
| | death | | | | | | |
| GO:0043085 | positive regulation of | 1392 | 1.94E−09 | 1 | 1 | 1 | 1 |
| | catalytic activity | | | | | | |
| GO:0043086 | negative regulation of | 795 | 0.036082289 | 1 | 1 | 1 | 1 |
| | catalytic activity | | | | | | |
| GO:0043087 | regulation of GTPase | 481 | 0.049277258 | 1 | 1 | 1 | 1 |
| | activity | | | | | | |
| GO:0043112 | receptor metabolic | 184 | 0.040035288 | 1 | 1 | 1 | 1 |
| | process | | | | | | |
| GO:0043122 | regulation of I-kappaB | 226 | 0.018065402 | 1 | 1 | 1 | 1 |
| | kinase/NF-kappaB | | | | | | |
| | signaling | | | | | | |
| GO:0043123 | positive regulation of | 181 | 0.00461387 | 1 | 1 | 1 | 1 |
| | I-kappaB kinase/NF- | | | | | | |
| | kappaB signaling | | | | | | |
| GO:0043207 | response to external | 984 | 3.89E−07 | 1 | 1 | 1 | 1 |
| | biotic stimulus | | | | | | |
| GO:0043249 | erythrocyte | 15 | 1 | 0.006823622 | 1 | 1 | 1 |
| | maturation | | | | | | |
| GO:0043280 | positive regulation of | 126 | 0.038843865 | 1 | 1 | 1 | 1 |
| | cysteine-type | | | | | | |
| | endopeptidase activity | | | | | | |
| | involved in apoptotic | | | | | | |
| | process | | | | | | |
| GO:0043281 | regulation of cysteine- | 209 | 0.007862201 | 1 | 1 | 1 | 1 |
| | type endopeptidase | | | | | | |
| | activity involved in | | | | | | |
| | apoptotic process | | | | | | |
| GO:0043299 | leukocyte | 531 | 3.32E−21 | 1 | 1 | 1 | 1 |
| | degranulation | | | | | | |
| GO:0043312 | neutrophil | 485 | 5.35E−20 | 1 | 1 | 1 | 1 |
| | degranulation | | | | | | |
| GO:0043405 | regulation of MAP | 335 | 2.37E−06 | 1 | 1 | 1 | 1 |
| | kinase activity | | | | | | |
| GO:0043406 | positive regulation of | 258 | 5.07E−06 | 1 | 1 | 1 | 1 |
| | MAP kinase activity | | | | | | |
| GO:0043408 | regulation of MAPK | 776 | 1.92E−05 | 1 | 1 | 1 | 1 |
| | cascade | | | | | | |
| GO:0043412 | macromolecule | 4269 | 8.87E−05 | 0.680217842 | 1 | 1 | 1 |
| | modification | | | | | | |
| GO:0043434 | response to peptide | 417 | 0.015891141 | 1 | 1 | 1 | 1 |
| | hormone | | | | | | |
| GO:0043549 | regulation of kinase | 837 | 5.71E−07 | 1 | 1 | 1 | 1 |
| | activity | | | | | | |
| GO:0044093 | positive regulation of | 1736 | 5.74E−10 | 1 | 1 | 1 | 1 |
| | molecular function | | | | | | |
| GO:0044237 | cellular metabolic | 11116 | 0.145299351 | 0.000447254 | 1 | 1 | 1 |
| | process | | | | | | |
| GO:0044238 | primary metabolic | 11112 | 1 | 0.007170056 | 1 | 1 | 1 |
| | process | | | | | | |
| GO:0044247 | cellular polysaccharide | 21 | 0.004611115 | 1 | 1 | 1 | 1 |
| | catabolic process | | | | | | |
| GO:0044248 | cellular catabolic | 2204 | 5.16E−08 | 0.87520387 | 1 | 1 | 1 |
| | process | | | | | | |
| GO:0044249 | cellular biosynthetic | 6015 | 1 | 5.07E−06 | 1 | 1 | 1 |
| | process | | | | | | |
| GO:0044260 | cellular | 8207 | 0.110332446 | 0.000171375 | 1 | 1 | 1 |
| | macromolecule | | | | | | |
| | metabolic process | | | | | | |
| GO:0044265 | cellular | 1118 | 0.015397049 | 0.748743161 | 1 | 1 | 1 |
| | macromolecule | | | | | | |
| | catabolic process | | | | | | |
| GO:0044267 | cellular protein | 5151 | 0.000173216 | 0.901236414 | 1 | 1 | 1 |
| | metabolic process | | | | | | |
| GO:0044271 | cellular nitrogen | 4905 | 1 | 1.09E−05 | 1 | 1 | 1 |
| | compound | | | | | | |
| | biosynthetic process | | | | | | |
| GO:0044275 | cellular carbohydrate | 40 | 0.021226881 | 1 | 1 | 1 | 1 |
| | catabolic process | | | | | | |
| GO:0045055 | regulated exocytosis | 788 | 1.08E−23 | 1 | 1 | 1 | 1 |
| GO:0045087 | innate immune | 908 | 1.30E−09 | 1 | 1 | 1 | 1 |
| | response | | | | | | |
| GO:0045088 | regulation of innate | 365 | 1.57E−07 | 1 | 1 | 1 | 1 |
| | immune response | | | | | | |
| GO:0045089 | positive regulation of | 309 | 4.50E−06 | 1 | 1 | 1 | 1 |
| | innate immune | | | | | | |
| | response | | | | | | |
| GO:0045123 | cellular extravasation | 54 | 0.000802516 | 1 | 1 | 1 | 1 |
| GO:0045184 | establishment of | 2079 | 2.55E−05 | 1 | 1 | 1 | 1 |
| | protein localization | | | | | | |
| GO:0045321 | leukocyte activation | 1257 | 4.94E−18 | 1 | 1 | 1 | 1 |
| GO:0045597 | positive regulation of | 974 | 0.020370955 | 0.831969884 | 1 | 1 | 1 |
| | cell differentiation | | | | | | |
| GO:0045785 | positive regulation of | 397 | 0.034129341 | 1 | 1 | 1 | 1 |
| | cell adhesion | | | | | | |
| GO:0045859 | regulation of protein | 762 | 1.59E−07 | 1 | 1 | 1 | 1 |
| | kinase activity | | | | | | |
| GO:0045860 | positive regulation of | 512 | 4.71E−09 | 1 | 1 | 1 | 1 |
| | protein kinase activity | | | | | | |
| GO:0045862 | positive regulation of | 352 | 0.001187493 | 1 | 1 | 1 | 1 |
| | proteolysis | | | | | | |
| GO:0045892 | negative regulation of | 1199 | 1 | 4.69E−09 | 1 | 0.469442217 | 1 |
| | transcription, DNA- | | | | | | |
| | templated | | | | | | |
| GO:0045932 | negative regulation of | 27 | 0.017009962 | 1 | 1 | 1 | 1 |
| | muscle contraction | | | | | | |
| GO:0045934 | negative regulation of | 1457 | 1 | 6.60E−07 | 1 | 0.923274969 | 1 |
| | nucleobase-containing | | | | | | |
| | compound metabolic | | | | | | |
| | process | | | | | | |
| GO:0045935 | positive regulation of | 1862 | 1 | 0.001568173 | 1 | 1 | 1 |
| | nucleobase-containing | | | | | | |
| | compound metabolic | | | | | | |
| | process | | | | | | |
| GO:0045937 | positive regulation of | 1124 | 3.91E−07 | 1 | 1 | 1 | 1 |
| | phosphate metabolic | | | | | | |
| | process | | | | | | |
| GO:0045987 | positive regulation of | 32 | 0.036082289 | 1 | 1 | 1 | 1 |
| | smooth muscle | | | | | | |
| | contraction | | | | | | |
| GO:0046483 | heterocycle metabolic | 6422 | 1 | 0.005792173 | 1 | 1 | 1 |
| | process | | | | | | |
| GO:0046649 | lymphocyte activation | 713 | 0.019063382 | 0.345762208 | 1 | 1 | 1 |
| GO:0046898 | response to | 4 | 1 | 0.013583116 | 1 | 1 | 1 |
| | cycloheximide | | | | | | |
| GO:0046903 | secretion | 1614 | 1.08E−23 | 1 | 1 | 1 | 1 |
| GO:0048010 | vascular endothelial | 91 | 0.038843865 | 1 | 1 | 1 | 1 |
| | growth factor | | | | | | |
| | receptor signaling | | | | | | |
| | pathway | | | | | | |
| GO:0048013 | ephrin receptor | 80 | 0.000841417 | 1 | 1 | 1 | 1 |
| | signaling pathway | | | | | | |
| GO:0048519 | negative regulation of | 5698 | 0.222729944 | 0.002085165 | 1 | 0.955645847 | 1 |
| | biological process | | | | | | |
| GO:0048522 | positive regulation of | 5330 | 2.61E−08 | 0.01290706 | 1 | 0.892839413 | 1 |
| | cellular process | | | | | | |
| GO:0048523 | negative regulation of | 4759 | 0.050048478 | 5.46E−05 | 1 | 0.681771571 | 1 |
| | cellular process | | | | | | |
| GO:0048534 | hematopoietic or | 861 | 0.009678209 | 0.000236607 | 1 | 1 | 0.533565531 |
| | lymphoid organ | | | | | | |
| | development | | | | | | |
| GO:0048583 | regulation of response | 4234 | 2.15E−08 | 1 | 1 | 0.400677758 | 1 |
| | to stimulus | | | | | | |
| GO:0048584 | positive regulation of | 2373 | 3.55E−10 | 1 | 1 | 1 | 1 |
| | response to stimulus | | | | | | |
| GO:0048821 | erythrocyte | 31 | 1 | 0.00036786 | 1 | 1 | 1 |
| | development | | | | | | |
| GO:0048870 | cell motility | 1688 | 6.00E−05 | 1 | 1 | 0.629638536 | 1 |
| GO:0048872 | homeostasis of | 248 | 0.019367226 | 0.052531007 | 1 | 1 | 1 |
| | number of cells | | | | | | |
| GO:0050663 | cytokine secretion | 210 | 0.008352331 | 1 | 1 | 1 | 1 |
| GO:0050701 | interleukin-1 secretion | 56 | 0.0280421 | 1 | 1 | 1 | 1 |
| GO:0050702 | interleukin-1 beta | 48 | 0.011788736 | 1 | 1 | 1 | 1 |
| | secretion | | | | | | |
| GO:0050776 | regulation of immune | 1022 | 2.37E−08 | 1 | 1 | 1 | 1 |
| | response | | | | | | |
| GO:0050778 | positive regulation of | 823 | 1.39E−07 | 1 | 1 | 1 | 1 |
| | immune response | | | | | | |
| GO:0050789 | regulation of | 11899 | 0.000110092 | 0.002209175 | 1 | 1 | 1 |
| | biological process | | | | | | |
| GO:0050790 | regulation of catalytic | 2281 | 2.61E−08 | 1 | 1 | 0.899460453 | 1 |
| | activity | | | | | | |
| GO:0050794 | regulation of cellular | 10828 | 4.85E−07 | 0.000104586 | 1 | 0.922242032 | 1 |
| | process | | | | | | |
| GO:0050817 | coagulation | 340 | 7.31E−09 | 1 | 1 | 1 | 1 |
| GO:0050818 | regulation of | 82 | 0.001058702 | 1 | 1 | 1 | 1 |
| | coagulation | | | | | | |
| GO:0050820 | positive regulation of | 26 | 0.014166664 | 1 | 1 | 1 | 1 |
| | coagulation | | | | | | |
| GO:0050863 | regulation of T cell | 305 | 0.015271277 | 1 | 1 | 1 | 1 |
| | activation | | | | | | |
| GO:0050865 | regulation of cell | 587 | 0.017981836 | 1 | 1 | 1 | 1 |
| | activation | | | | | | |
| GO:0050866 | negative regulation of | 184 | 0.040035288 | 1 | 1 | 1 | 1 |
| | cell activation | | | | | | |
| GO:0050878 | regulation of body | 493 | 1.93E−05 | 1 | 1 | 1 | 0.966588903 |
| | fluid levels | | | | | | |
| GO:0050900 | leukocyte migration | 478 | 0.000176302 | 1 | 1 | 1 | 1 |
| GO:0051050 | positive regulation of | 936 | 0.016217447 | 1 | 1 | 1 | 1 |
| | transport | | | | | | |
| GO:0051090 | regulation of DNA- | 412 | 0.026984947 | 0.792082077 | 1 | 1 | 1 |
| | binding transcription | | | | | | |
| | factor activity | | | | | | |
| GO:0051091 | positive regulation of | 255 | 0.004012175 | 0.922242032 | 1 | 1 | 1 |
| | DNA-binding | | | | | | |
| | transcription factor | | | | | | |
| | activity | | | | | | |
| GO:0051092 | positive regulation of | 146 | 1.92E−05 | 0.922242032 | 1 | 1 | 1 |
| | NF-kappaB | | | | | | |
| | transcription factor | | | | | | |
| | activity | | | | | | |
| GO:0051094 | positive regulation of | 1395 | 0.038843865 | 0.830036129 | 1 | 1 | 1 |
| | developmental | | | | | | |
| | process | | | | | | |
| GO:0051128 | regulation of cellular | 2457 | 3.72E−05 | 0.996446813 | 1 | 1 | 1 |
| | component | | | | | | |
| | organization | | | | | | |
| GO:0051130 | positive regulation of | 1210 | 4.29E−05 | 1 | 1 | 1 | 1 |
| | cellular component | | | | | | |
| | organization | | | | | | |
| GO:0051171 | regulation of nitrogen | 5907 | 0.008673409 | 5.89E−05 | 1 | 1 | 1 |
| | compound metabolic | | | | | | |
| | process | | | | | | |
| GO:0051172 | negative regulation of | 2410 | 0.348615674 | 8.40E−06 | 1 | 1 | 1 |
| | nitrogen compound | | | | | | |
| | metabolic process | | | | | | |
| GO:0051173 | positive regulation of | 3140 | 2.20E−05 | 0.072497165 | 1 | 1 | 1 |
| | nitrogen compound | | | | | | |
| | metabolic process | | | | | | |
| GO:0051174 | regulation of | 1738 | 7.55E−07 | 1 | 1 | 1 | 1 |
| | phosphorus metabolic | | | | | | |
| | process | | | | | | |
| GO:0051222 | positive regulation of | 402 | 0.001684062 | 1 | 1 | 1 | 1 |
| | protein transport | | | | | | |
| GO:0051223 | regulation of protein | 672 | 0.023649461 | 1 | 1 | 1 | 1 |
| | transport | | | | | | |
| GO:0051246 | regulation of protein | 2860 | 6.36E−08 | 1 | 1 | 1 | 1 |
| | metabolic process | | | | | | |
| GO:0051247 | positive regulation of | 1681 | 1.05E−09 | 1 | 1 | 1 | 1 |
| | protein metabolic | | | | | | |
| | process | | | | | | |
| GO:0051248 | negative regulation of | 1144 | 0.008981555 | 0.996446813 | 1 | 1 | 1 |
| | protein metabolic | | | | | | |
| | process | | | | | | |
| GO:0051252 | regulation of RNA | 3738 | 1 | 2.45E−06 | 1 | 1 | 1 |
| | metabolic process | | | | | | |
| GO:0051253 | negative regulation of | 1330 | 1 | 3.23E−08 | 1 | 0.633161182 | 1 |
| | RNA metabolic | | | | | | |
| | process | | | | | | |
| GO:0051254 | positive regulation of | 1680 | 1 | 0.001413106 | 1 | 1 | 1 |
| | RNA metabolic | | | | | | |
| | process | | | | | | |
| GO:0051272 | positive regulation of | 553 | 7.10E−06 | 1 | 1 | 1 | 1 |
| | cellular component | | | | | | |
| | movement | | | | | | |
| GO:0051336 | regulation of | 1281 | 0.001796452 | 1 | 1 | 1 | 1 |
| | hydrolase activity | | | | | | |
| GO:0051338 | regulation of | 941 | 2.18E−06 | 1 | 1 | 1 | 1 |
| | transferase activity | | | | | | |
| GO:0051345 | positive regulation of | 763 | 0.002761855 | 1 | 1 | 1 | 1 |
| | hydrolase activity | | | | | | |
| GO:0051347 | positive regulation of | 626 | 3.58E−08 | 1 | 1 | 1 | 1 |
| | transferase activity | | | | | | |
| GO:0051607 | defense response to | 228 | 0.007642349 | 1 | 1 | 1 | 1 |
| | virus | | | | | | |
| GO:0051641 | cellular localization | 2847 | 0.024959734 | 1 | 1 | 1 | 1 |
| GO:0051674 | localization of cell | 1688 | 6.00E−05 | 1 | 1 | 0.629638536 | 1 |
| GO:0051704 | multi-organism | 2485 | 3.72E−05 | 1 | 1 | 1 | 1 |
| | process | | | | | | |
| GO:0051707 | response to other | 982 | 3.62E−07 | 1 | 1 | 1 | 1 |
| | organism | | | | | | |
| GO:0051716 | cellular response to | 7432 | 3.67E−09 | 0.87520387 | 1 | 0.07488625 | 1 |
| | stimulus | | | | | | |
| GO:0060079 | excitatory | 103 | 1 | 1 | 1 | 0.026564194 | 1 |
| | postsynaptic potential | | | | | | |
| GO:0060218 | hematopoietic stem | 34 | 1 | 0.035278512 | 1 | 1 | 1 |
| | cell differentiation | | | | | | |
| GO:0060255 | regulation of | 6630 | 0.443316572 | 0.00566515 | 1 | 1 | 1 |
| | macromolecule | | | | | | |
| | metabolic process | | | | | | |
| GO:0060319 | primitive erythrocyte | 4 | 1 | 0.013583116 | 1 | 1 | 1 |
| | differentiation | | | | | | |
| GO:0060326 | cell chemotaxis | 295 | 0.010056469 | 1 | 1 | 1 | 1 |
| GO:0060341 | regulation of cellular | 879 | 0.024731895 | 1 | 1 | 1 | 1 |
| | localization | | | | | | |
| GO:0060627 | regulation of vesicle- | 498 | 0.021966072 | 1 | 1 | 1 | 1 |
| | mediated transport | | | | | | |
| GO:0060969 | negative regulation of | 35 | 1 | 0.040035288 | 1 | 1 | 1 |
| | gene silencing | | | | | | |
| GO:0061003 | positive regulation of | 19 | 1 | 1 | 1 | 1 | 0.035229966 |
| | dendritic spine | | | | | | |
| | morphogenesis | | | | | | |
| GO:0061024 | membrane | 846 | 0.012347546 | 1 | 1 | 1 | 1 |
| | organization | | | | | | |
| GO:0061041 | regulation of wound | 134 | 0.022218406 | 1 | 1 | 1 | 1 |
| | healing | | | | | | |
| GO:0061298 | retina vasculature | 18 | 1 | 1 | 1 | 1 | 0.031028127 |
| | development in | | | | | | |
| | camera-type eye | | | | | | |
| GO:0061515 | myeloid cell | 67 | 0.52528845 | 7.08E−05 | 1 | 1 | 1 |
| | development | | | | | | |
| GO:0061900 | glial cell activation | 40 | 0.003561051 | 1 | 1 | 1 | 1 |
| GO:0061919 | process utilizing | 466 | 0.003701303 | 1 | 1 | 1 | 1 |
| | autophagic | | | | | | |
| | mechanism | | | | | | |
| GO:0065007 | biological regulation | 12576 | 1.85E−06 | 0.018565448 | 1 | 0.356365961 | 1 |
| GO:0065009 | regulation of | 3187 | 1.56E−06 | 1 | 1 | 0.478489879 | 1 |
| | molecular function | | | | | | |
| GO:0070201 | regulation of | 715 | 0.011042006 | 1 | 1 | 1 | 1 |
| | establishment of | | | | | | |
| | protein localization | | | | | | |
| GO:0070340 | detection of bacterial | 3 | 0.005055861 | 1 | 1 | 1 | 1 |
| | lipopeptide | | | | | | |
| GO:0070498 | interleukin-1- | 55 | 0.025766984 | 1 | 1 | 1 | 1 |
| | mediated signaling | | | | | | |
| | pathway | | | | | | |
| GO:0070527 | platelet aggregation | 57 | 0.031006288 | 1 | 1 | 1 | 1 |
| GO:0070727 | cellular | 1840 | 0.00087738 | 1 | 1 | 1 | 1 |
| | macromolecule | | | | | | |
| | localization | | | | | | |
| GO:0070887 | cellular response to | 3144 | 7.43E−11 | 0.481144832 | 1 | 1 | 0.916075135 |
| | chemical stimulus | | | | | | |
| GO:0071216 | cellular response to | 229 | 2.35E−05 | 1 | 1 | 1 | 1 |
| | biotic stimulus | | | | | | |
| GO:0071219 | cellular response to | 206 | 0.000693746 | 1 | 1 | 1 | 1 |
| | molecule of bacterial | | | | | | |
| | origin | | | | | | |
| GO:0071222 | cellular response to | 199 | 0.012917217 | 1 | 1 | 1 | 1 |
| | lipopolysaccharide | | | | | | |
| GO:0071260 | cellular response to | 78 | 1.92E−05 | 1 | 1 | 1 | 1 |
| | mechanical stimulus | | | | | | |
| GO:0071310 | cellular response to | 2595 | 9.32E−10 | 0.179921038 | 1 | 1 | 0.883468397 |
| | organic substance | | | | | | |
| GO:0071345 | cellular response to | 1028 | 1.59E−07 | 1 | 1 | 1 | 1 |
| | cytokine stimulus | | | | | | |
| GO:0071346 | cellular response to | 178 | 0.030521352 | 1 | 1 | 1 | 1 |
| | interferon-gamma | | | | | | |
| GO:0071375 | cellular response to | 307 | 0.016372759 | 1 | 1 | 1 | 1 |
| | peptide hormone | | | | | | |
| | stimulus | | | | | | |
| GO:0071417 | cellular response to | 555 | 7.77E−06 | 1 | 1 | 1 | 0.519887215 |
| | organonitrogen | | | | | | |
| | compound | | | | | | |
| GO:0071496 | cellular response to | 328 | 1.37E−05 | 0.133254818 | 1 | 1 | 1 |
| | external stimulus | | | | | | |
| GO:0071621 | granulocyte | 120 | 0.002310269 | 1 | 1 | 1 | 1 |
| | chemotaxis | | | | | | |
| GO:0071622 | regulation of | 42 | 0.026791027 | 1 | 1 | 1 | 1 |
| | granulocyte | | | | | | |
| | chemotaxis | | | | | | |
| GO:0071675 | regulation of | 42 | 0.026791027 | 1 | 1 | 1 | 1 |
| | mononuclear cell | | | | | | |
| | migration | | | | | | |
| GO:0071702 | organic substance | 2745 | 0.000324796 | 1 | 1 | 1 | 0.739725387 |
| | transport | | | | | | |
| GO:0071705 | nitrogen compound | 2335 | 0.001910279 | 1 | 1 | 1 | 1 |
| | transport | | | | | | |
| GO:0071706 | tumor necrosis factor | 135 | 0.007547433 | 1 | 1 | 1 | 1 |
| | superfamily cytokine | | | | | | |
| | production | | | | | | |
| GO:0071840 | cellular component | 6506 | 0.075463572 | 0.686175641 | 1 | 0.698373195 | 0.046322431 |
| | organization or | | | | | | |
| | biogenesis | | | | | | |
| GO:0071900 | regulation of protein | 496 | 1.59E−07 | 1 | 1 | 1 | 1 |
| | serine/threonine | | | | | | |
| | kinase activity | | | | | | |
| GO:0071902 | positive regulation of | 323 | 2.62E−08 | 1 | 1 | 1 | 1 |
| | protein | | | | | | |
| | serine/threonine | | | | | | |
| | kinase activity | | | | | | |
| GO:0072672 | neutrophil | 10 | 0.001140246 | 1 | 1 | 1 | 1 |
| | extravasation | | | | | | |
| GO:0080090 | regulation of primary | 6069 | 0.004520227 | 9.52E−06 | 1 | 1 | 1 |
| | metabolic process | | | | | | |
| GO:0080134 | regulation of response | 1452 | 6.85E−08 | 1 | 1 | 1 | 1 |
| | to stress | | | | | | |
| GO:0080135 | regulation of cellular | 684 | 0.009359443 | 1 | 1 | 1 | 1 |
| | response to stress | | | | | | |
| GO:0090087 | regulation of peptide | 701 | 0.0256708 | 1 | 1 | 1 | 1 |
| | transport | | | | | | |
| GO:0090304 | nucleic acid metabolic | 5634 | 1 | 0.001513988 | 1 | 1 | 1 |
| | process | | | | | | |
| GO:0090316 | positive regulation of | 158 | 0.010740006 | 1 | 1 | 1 | 1 |
| | intracellular protein | | | | | | |
| | transport | | | | | | |
| GO:0097529 | myeloid leukocyte | 199 | 0.000433484 | 1 | 1 | 1 | 1 |
| | migration | | | | | | |
| GO:0097530 | granulocyte migration | 134 | 0.000500171 | 1 | 1 | 1 | 1 |
| GO:0097659 | nucleic acid- | 3642 | 1 | 3.98E−06 | 1 | 1 | 1 |
| | templated | | | | | | |
| | transcription | | | | | | |
| GO:0098542 | defense response to | 548 | 0.041421832 | 1 | 1 | 1 | 1 |
| | other organism | | | | | | |
| GO:0098543 | detection of other | 13 | 0.049467321 | 1 | 1 | 1 | 1 |
| | organism | | | | | | |
| GO:0098657 | import into cell | 900 | 1.17E−05 | 1 | 1 | 0.996446813 | 1 |
| GO:0098771 | inorganic ion | 722 | 0.039539873 | 1 | 1 | 0.875764979 | 1 |
| | homeostasis | | | | | | |
| GO:0098976 | excitatory chemical | 8 | 1 | 1 | 1 | 0.013930431 | 1 |
| | synaptic transmission | | | | | | |
| GO:0099565 | chemical synaptic | 111 | 1 | 1 | 1 | 0.037418398 | 1 |
| | transmission, | | | | | | |
| | postsynaptic | | | | | | |
| GO:0150076 | neuroinflammatory | 47 | 0.0002357 | 1 | 1 | 1 | 1 |
| | response | | | | | | |
| GO:1900046 | regulation of | 77 | 0.002919532 | 1 | 1 | 1 | 1 |
| | hemostasis | | | | | | |
| GO:1901224 | positive regulation of | 70 | 0.026584093 | 1 | 1 | 1 | 1 |
| | NIK/NF-kappaB | | | | | | |
| | signaling | | | | | | |
| GO:1901360 | organic cyclic | 6676 | 1 | 0.011793821 | 1 | 1 | 1 |
| | compound metabolic | | | | | | |
| | process | | | | | | |
| GO:1901362 | organic cyclic | 4387 | 1 | 1.89E−05 | 1 | 1 | 1 |
| | compound | | | | | | |
| | biosynthetic process | | | | | | |
| GO:1901564 | organonitrogen | 6899 | 9.53E−05 | 1 | 1 | 1 | 1 |
| | compound metabolic | | | | | | |
| | process | | | | | | |
| GO:1901565 | organonitrogen | 1253 | 0.000953023 | 1 | 1 | 1 | 1 |
| | compound catabolic | | | | | | |
| | process | | | | | | |
| GO:1901575 | organic substance | 2071 | 3.56E−07 | 1 | 1 | 1 | 1 |
| | catabolic process | | | | | | |
| GO:1901576 | organic substance | 6099 | 1 | 2.53E−06 | 1 | 1 | 1 |
| | biosynthetic process | | | | | | |
| GO:1901652 | response to peptide | 481 | 0.000512427 | 1 | 1 | 1 | 1 |
| GO:1901653 | cellular response to | 356 | 0.000545066 | 1 | 1 | 1 | 1 |
| | peptide | | | | | | |
| GO:1901698 | response to nitrogen | 1037 | 2.06E−05 | 1 | 1 | 1 | 1 |
| | compound | | | | | | |
| GO:1901699 | cellular response to | 612 | 1.40E−05 | 1 | 1 | 1 | 0.402781503 |
| | nitrogen compound | | | | | | |
| GO:1901700 | response to oxygen- | 1577 | 1.54E−05 | 1 | 1 | 1 | 1 |
| | containing compound | | | | | | |
| GO:1901701 | cellular response to | 1092 | 5.13E−05 | 1 | 1 | 1 | 0.795980333 |
| | oxygen-containing | | | | | | |
| | compound | | | | | | |
| GO:1902275 | regulation of | 175 | 1 | 0.038417362 | 1 | 1 | 1 |
| | chromatin | | | | | | |
| | organization | | | | | | |
| GO:1902531 | regulation of | 1881 | 1.13E−09 | 1 | 1 | 1 | 1 |
| | intracellular signal | | | | | | |
| | transduction | | | | | | |
| GO:1902533 | positive regulation of | 1077 | 3.91E−07 | 1 | 1 | 1 | 1 |
| | intracellular signal | | | | | | |
| | transduction | | | | | | |
| GO:1902622 | regulation of | 31 | 0.03127218 | 1 | 1 | 1 | 1 |
| | neutrophil migration | | | | | | |
| GO:1902679 | negative regulation of | 1251 | 1 | 1.26E−08 | 1 | 0.604011244 | 1 |
| | RNA biosynthetic | | | | | | |
| | process | | | | | | |
| GO:1903037 | regulation of | 293 | 0.021687327 | 1 | 1 | 1 | 1 |
| | leukocyte cell-cell | | | | | | |
| | adhesion | | | | | | |
| GO:1903506 | regulation of nucleic | 3499 | 1 | 2.97E−06 | 1 | 1 | 1 |
| | acid-templated | | | | | | |
| | transcription | | | | | | |
| GO:1903507 | negative regulation of | 1249 | 1 | 1.21E−08 | 1 | 0.598967854 | 1 |
| | nucleic acid- | | | | | | |
| | templated | | | | | | |
| | transcription | | | | | | |
| GO:1903510 | mucopolysaccharide | 110 | 0.014483105 | 1 | 1 | 1 | 1 |
| | metabolic process | | | | | | |
| GO:1903555 | regulation of tumor | 131 | 0.01844494 | 1 | 1 | 1 | 1 |
| | necrosis factor | | | | | | |
| | superfamily cytokine | | | | | | |
| | production | | | | | | |
| GO:1903557 | positive regulation of | 76 | 0.04283723 | 1 | 1 | 1 | 1 |
| GO:1903747 | tumor necrosis factor | | | | | | |
| | superfamily cytokine | | | | | | |
| | production | | | | | | |
| | regulation of | 70 | 0.026584093 | 1 | 1 | 1 | 1 |
| | establishment of | | | | | | |
| | protein localization to | | | | | | |
| | mitochondrion | | | | | | |
| GO:1903749 | positive regulation of | 56 | 0.0280421 | 1 | 1 | 1 | 1 |
| | establishment of | | | | | | |
| | protein localization to | | | | | | |
| | mitochondrion | | | | | | |
| GO:1903827 | regulation of cellular | 508 | 0.000254902 | 1 | 1 | 1 | 1 |
| | protein localization | | | | | | |
| GO:1903829 | positive regulation of | 318 | 0.004402893 | 1 | 1 | 1 | 1 |
| | cellular protein | | | | | | |
| | localization | | | | | | |
| GO:1904951 | positive regulation of | 437 | 0.000496601 | 1 | 1 | 1 | 1 |
| | establishment of | | | | | | |
| | protein localization | | | | | | |
| GO:1905268 | negative regulation of | 60 | 1 | 0.028370428 | 1 | 1 | 1 |
| | chromatin | | | | | | |
| | organization | | | | | | |
| GO:1990266 | neutrophil migration | 112 | 0.001132309 | 1 | 1 | 1 | 1 |
| GO:1990928 | response to amino | 47 | 0.046835063 | 1 | 1 | 1 | 1 |
| | acid starvation | | | | | | |
| GO:2000112 | regulation of cellular | 3918 | 1 | 1.43E−05 | 1 | 1 | 1 |
| | macromolecule | | | | | | |
| | biosynthetic process | | | | | | |
| GO:2000113 | negative regulation of | 1459 | 1 | 3.40E−07 | 1 | 1 | 1 |
| | cellular | | | | | | |
| | macromolecule | | | | | | |
| | biosynthetic process | | | | | | |
| GO:2000116 | regulation of cysteine- | 232 | 0.00334288 | 1 | 1 | 1 | 1 |
| | type endopeptidase | | | | | | |
| | activity | | | | | | |
| GO:2000145 | regulation of cell | 968 | 0.00318429 | 1 | 1 | 0.995169577 | 1 |
| | motility | | | | | | |
| GO:2000147 | positive regulation of | 539 | 9.45E−06 | 1 | 1 | 1 | 1 |
| | cell motility | | | | | | |
| GO:2000377 | regulation of reactive | 185 | 0.041941486 | 1 | 1 | 1 | 1 |
| | oxygen species | | | | | | |
| | metabolic process | | | | | | |
| GO:2000463 | positive regulation of | 27 | 1 | 1 | 1 | 0.032162807 | 1 |
| | excitatory | | | | | | |
| | postsynaptic potential | | | | | | |
| GO:2001056 | positive regulation of | 143 | 0.035857337 | 1 | 1 | 0.716801532 | 1 |
| | cysteine-type | | | | | | |
| | endopeptidase activity | | | | | | |
| GO:2001141 | regulation of RNA | 3507 | 1 | 3.35E−06 | 1 | 1 | 1 |
| | biosynthetic process | | | | | | |
| GO:2001233 | regulation of | 391 | 0.014166664 | 1 | 1 | 1 | 1 |
| | apoptotic signaling | | | | | | |
| | pathway | | | | | | |

Time Series Analysis of Synovial Cell Marker Genes in RA Flares

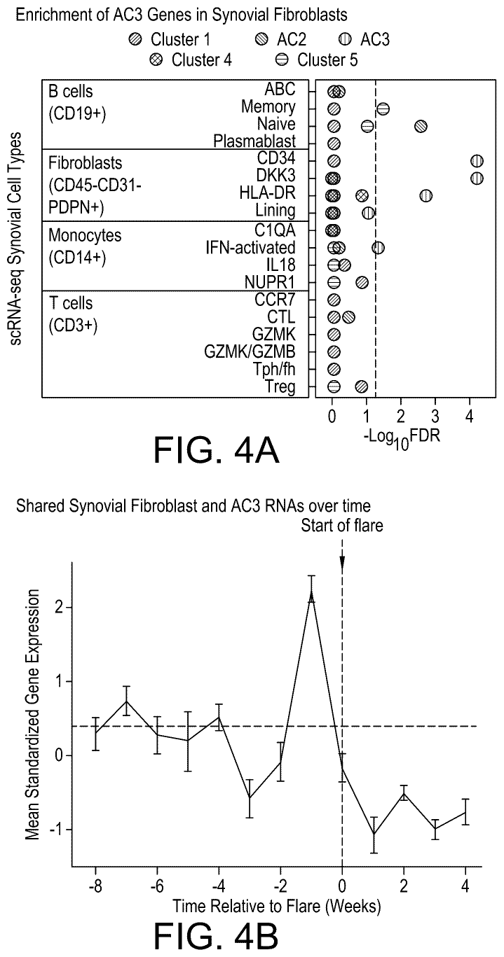

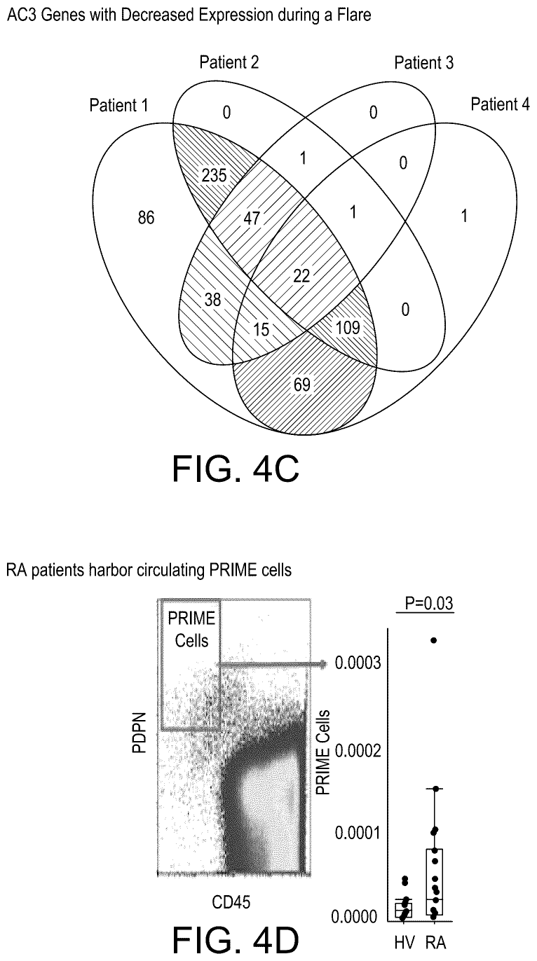

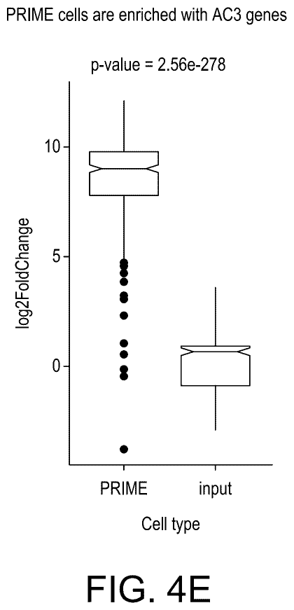

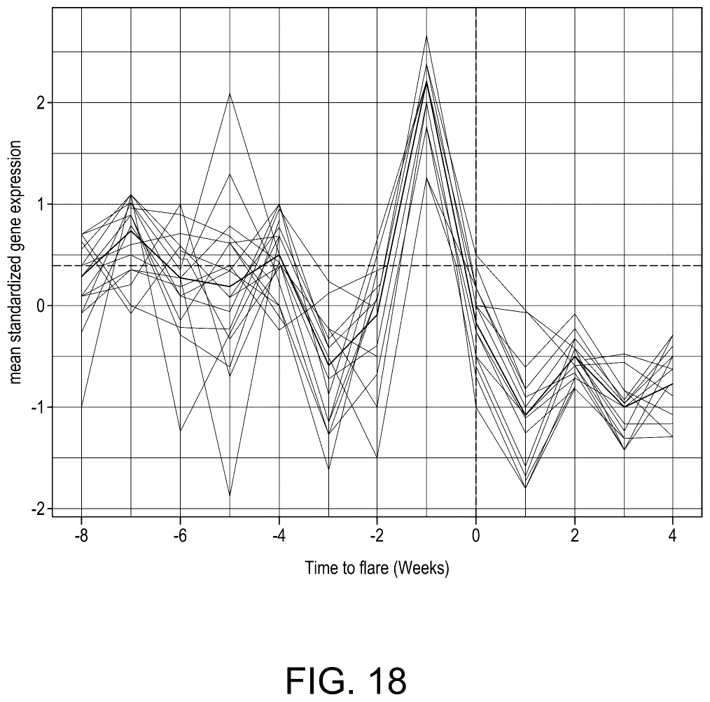

To better characterize the relevance of the clusters identified by the time series analysis to synovitis (FIG. 3C), we examined them for enrichment in synovial cell subtypes characterized by scRNAseq. This analysis of 5265 single RA and osteoarthritis patient synovial cells identified four fibroblast, four B cell, six T cell, and four monocyte subpopulations (FIG. 4A). We identified approximately 200 marker genes that best distinguished each of 18 synovial cell types. AC2 was enriched with naive B cell genes (FIG. 4A and FIG. 17), and AC3 was enriched with three sublining fibroblast genes (CD34+, HLA-DR+, and DKK3+) (FIG. 4A). Two of these fibroblast panels, CD34+ and HLA-DR+, are more abundant in inflamed synovium (20). We plotted expression of those transcripts that were common to both synovial sublining fibroblasts and AC3 over time and again noted their increased expression in blood one week prior to flare and decreased expression during flare (FIG. 4B, FIG. 18, and Table 5).

Overall, 622 of 625 AC3 genes decreased during flare in patient 1, and a panel (194 genes) also decreased in flares from at least 3 out of 4 RA patients (and 22 genes in 4 out of 4 patients; FIG. 4C and Table 6), and permutation test indicated this overlap was greater than expected by chance (p=0.0001). Pathway analysis of the panel of 194 overlapping genes was again enriched for extracellular matrix and secreted glycoprotein.

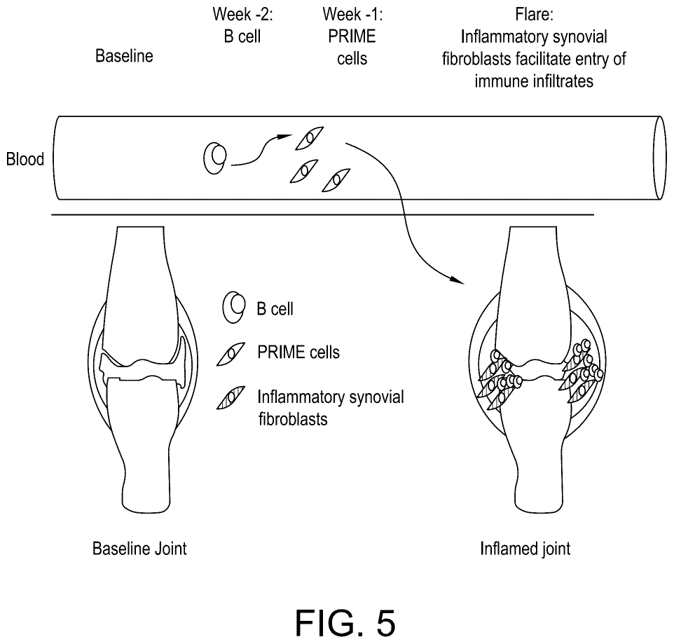

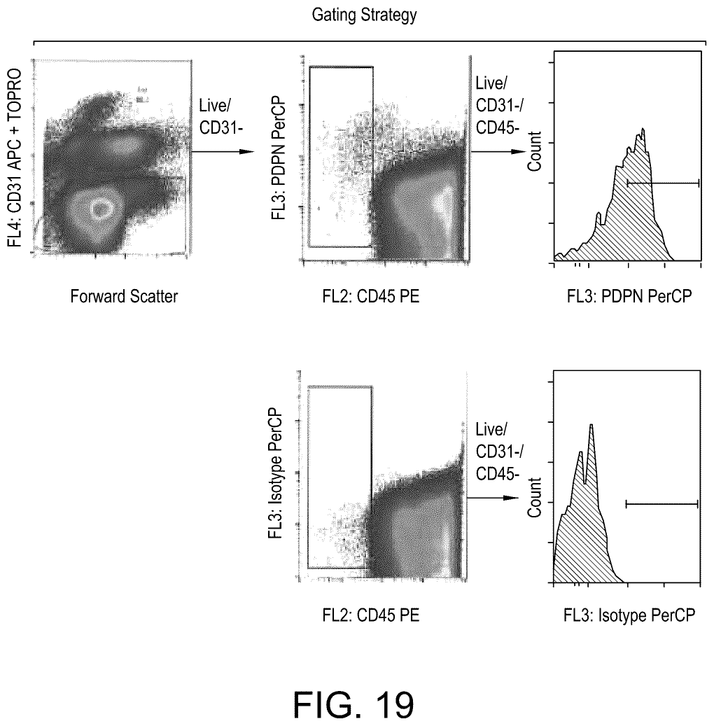

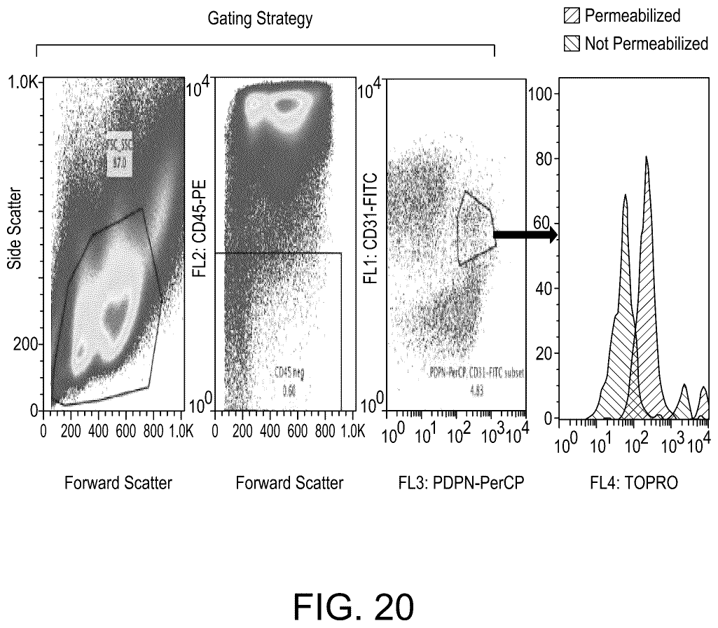

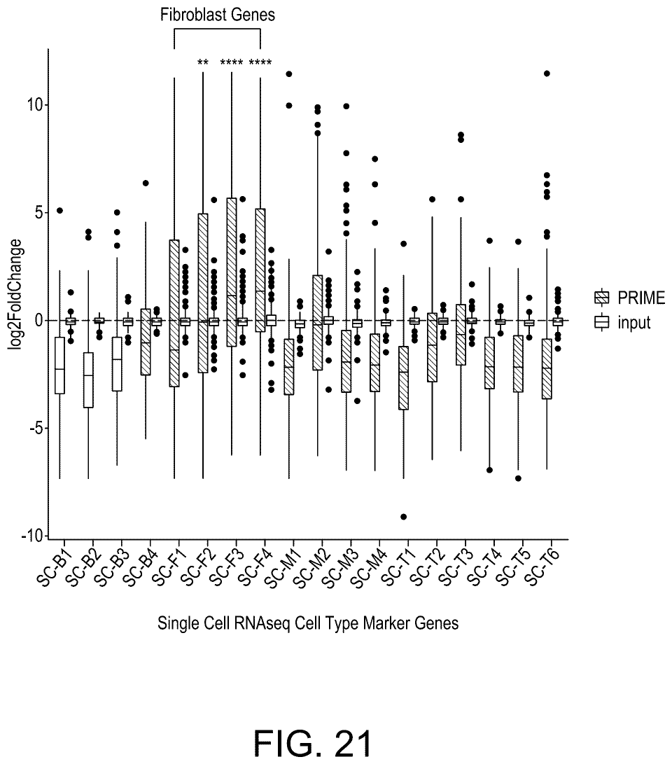

We further tested whether cells that expressed surface markers of synovial fibroblasts were detectable in RA blood by flow cytometry (FIGS. 19 and 20). CD45−/CD31−/PDPN+ cells were increased in 19 additional RA patient blood relative to healthy controls (FIG. 4D). RNAseq of these cells confirmed they were enriched with AC3 cluster genes (FIG. 4E), synovial fibroblast genes (FIG. 21). Given their expression of classical mesenchymal surface markers and genes, we refer to these as PRe-Inflammatory Mesenchymal Cells (PRIME cells). Taken together, our observations suggest a model in which sequential activation of B cells activate PRIME cells just prior to flares, which are then evident at flare in inflamed synovium as inflammatory sublining fibroblasts (FIG. 5).

### Discussion

We present longitudinal genomics as a strategy to study the antecedents to RA flare that may be generalizable to autoimmune diseases associated with waxing/waning clinical courses. We developed easy-to-use tools for patients to acquire both quantifiable clinical symptoms and molecular data at home over many years. This allowed us to capture data prior to the onset of clinical flares and retrospectively analyze it, identifying different RNA signatures (AC2 and AC3) evident in peripheral blood 1-2 weeks prior to flare.

The RNA signature of AC3 and sorted CD45−/CD31−/PDPN+ circulating cells revealed enrichment for pathways including cartilage morphogenesis, endochondral bone growth, and extracellular matrix organization (FIG. 3E) and strongly overlapped with synovial sublining fibroblasts. We therefore propose antecedent PRIME cells are the precursors to inflammatory sublining fibroblasts previously found adjacent to blood vessels in inflamed RA synovium (21).

Significantly, inflamed sublining fibroblasts are pathogenic in an animal model of arthritis (22). Our discovery that human AC3 genes share molecular characteristics of sublining fibroblasts, together with the observation that these cells spike prior to flare but are less detectable in blood during flare (FIGS. 2 and 4) support a model in which PRIME cells immigrate acutely from blood to the synovium where they contribute to the inflammatory process (FIG. 5). This model is consistent with the observation that RA synovial fibroblasts can traffic to cartilage implants and are sufficient to passively transfer synovial inflammation in mice (23). Together our data suggest the mesenchymal signal detected in AC3 prior to flares represent a previously uncharacterized type of trafficking fibroblast that circulates in blood.

In addition, we observed a second RNA signature, AC2, activated in blood prior to the spike in AC3. AC2 bear RNA hallmarks of naive B cells. This finding is reminiscent of recent studies demonstrating autoreactive naive B cells are specifically activated in RA patients (24). While the triggers of these are unknown, infectious (for example bacterial or viral antigens), environmental or endogenous toxins (25-27) could provide a source of either specific antigens or activate pattern recognition receptors.

In conclusion, we demonstrate methods for densely collecting longitudinal clinical and gene expression data that can be used to discover changes in transcriptional profiles in the blood weeks prior to symptom onset. This approach led to discovery of PRIME cells, bearing hallmarks of synovial fibroblasts, which are more common in RA patients and increase in blood just prior to flares. In modeling all our data (FIG. 5), we suggest that prior to clinical flare, systemic B cell immune activation (detected as AC2) acts on PRIME cells, which traffic to the blood (detected as AC3) and subsequently to the synovial sublining during flares of disease activity. More generally, this work in RA provides an exemplar of an approach to waxing/waning inflammatory disease, suggesting a general strategy relevant to additional disorders such as lupus, multiple sclerosis, and vasculitis.

Tables 2, 5 and 6 referenced in the above are provided as follows.

TABLE 2
Pathway Analysis of Differentially Expressed Genes in Flare Versus Baseline

| ID | TERM | nGenes | sig_up.ngenes | sig_down.ngenes | fdr.up | fdr.down |
|---|---|---|---|---|---|---|
| GO:0002274 | myeloid leukocyte activation | 607 | 112 | 3 | 1.78E−29 | 1 |
| GO:0006955 | immune response | 1725 | 205 | 16 | 8.83E−29 | 1 |
| GO:0032940 | secretion by cell | 1354 | 176 | 12 | 8.83E−29 | 1 |
| GO:0043299 | leukocyte degranulation | 510 | 100 | 3 | 1.04E−28 | 1 |
| GO:0036230 | granulocyte activation | 486 | 97 | 3 | 1.74E−28 | 1 |
| GO:0001775 | cell activation | 1250 | 166 | 14 | 2.26E−28 | 1 |
| GO:0002275 | myeloid cell activation involved in immune response | 520 | 100 | 3 | 2.26E−28 | 1 |
| GO:0002376 | immune system process | 2531 | 260 | 31 | 2.26E−28 | 1 |
| GO:0002446 | neutrophil mediated immunity | 482 | 96 | 3 | 2.26E−28 | 1 |
| GO:0042119 | neutrophil activation | 480 | 96 | 3 | 2.26E−28 | 1 |
| GO:0046903 | secretion | 1468 | 183 | 13 | 2.41E−28 | 1 |
| GO:0043312 | neutrophil degranulation | 469 | 94 | 3 | 5.05E−28 | 1 |
| GO:0002444 | myeloid leukocyte mediated immunity | 527 | 100 | 3 | 5.52E−28 | 1 |
| GO:0002283 | neutrophil activation involved in immune response | 472 | 94 | 3 | 7.33E−28 | 1 |
| GO:0002366 | leukocyte activation involved in immune response | 652 | 111 | 5 | 4.28E−27 | 1 |
| GO:0002263 | cell activation involved in immune response | 655 | 111 | 5 | 6.10E−27 | 1 |
| GO:0045321 | leukocyte activation | 1111 | 150 | 10 | 2.96E−26 | 1 |
| GO:0006887 | exocytosis | 857 | 128 | 5 | 4.51E−26 | 1 |
| GO:0002443 | leukocyte mediated immunity | 726 | 116 | 5 | 5.52E−26 | 1 |
| GO:0002252 | immune effector process | 1063 | 145 | 7 | 8.77E−26 | 1 |
| GO:0045055 | regulated exocytosis | 755 | 118 | 4 | 1.23E−25 | 1 |
| GO:0016192 | vesicle-mediated transport | 1859 | 186 | 26 | 1.74E−17 | 1 |
| GO:0006950 | response to stress | 3333 | 273 | 39 | 5.46E−15 | 1 |
| GO:0050896 | response to stimulus | 7448 | 500 | 104 | 5.71E−15 | 1 |
| GO:0023052 | signaling | 5257 | 380 | 73 | 5.88E−14 | 1 |
| GO:0007165 | signal transduction | 4852 | 356 | 65 | 1.41E−13 | 1 |
| GO:0007154 | cell communication | 5297 | 379 | 74 | 3.76E−13 | 1 |
| GO:0009611 | response to wounding | 571 | 78 | 17 | 1.05E−12 | 0.129037518 |
| GO:0002684 | positive regulation of immune system process | 896 | 104 | 9 | 1.19E−12 | 1 |
| GO:0048584 | positive regulation of response to stimulus | 1994 | 181 | 22 | 1.19E−12 | 1 |
| GO:0051716 | cellular response to stimulus | 6105 | 420 | 85 | 1.63E−12 | 1 |
| GO:0002682 | regulation of immune system process | 1283 | 130 | 12 | 9.76E−12 | 1 |
| GO:0002253 | activation of immune response | 494 | 69 | 1 | 1.47E−11 | 1 |
| GO:0050776 | regulation of immune response | 831 | 96 | 8 | 1.89E−11 | 1 |
| GO:0050778 | positive regulation of immune response | 653 | 82 | 5 | 1.89E−11 | 1 |
| GO:0070887 | cellular response to chemical stimulus | 2747 | 224 | 40 | 2.59E−11 | 1 |
| GO:0045087 | innate immune response | 695 | 85 | 2 | 2.61E−11 | 1 |
| GO:0006952 | defense response | 1291 | 129 | 6 | 3.05E−11 | 1 |
| GO:0042060 | wound healing | 470 | 66 | 16 | 3.70E−11 | 0.059726283 |
| GO:0044093 | positive regulation of molecular function | 1569 | 147 | 20 | 6.93E−11 | 1 |
| GO:0080134 | regulation of response to stress | 1248 | 124 | 5 | 1.45E−10 | 1 |
| GO:0048583 | regulation of response to stimulus | 3614 | 272 | 43 | 2.27E−10 | 1 |
| GO:0050794 | regulation of cellular process | 9002 | 557 | 112 | 3.98E−10 | 1 |
| GO:0002757 | immune response-activating signal transduction | 426 | 60 | 1 | 4.31E−10 | 1 |
| GO:0048518 | positive regulation of biological process | 5233 | 362 | 71 | 4.36E−10 | 1 |
| GO:0006810 | transport | 4550 | 324 | 60 | 4.63E−10 | 1 |
| GO:0043085 | positive regulation of catalytic activity | 1257 | 123 | 17 | 4.63E−10 | 1 |
| GO:0065007 | biological regulation | 10244 | 616 | 138 | 4.63E−10 | 1 |
| GO:0051234 | establishment of localization | 4659 | 330 | 64 | 4.65E−10 | 1 |
| GO:0071310 | cellular response to organic substance | 2268 | 189 | 35 | 5.77E−10 | 1 |
| GO:0002764 | immune response-regulating signaling pathway | 455 | 62 | 2 | 6.66E−10 | 1 |
| GO:0035556 | intracellular signal transduction | 2501 | 202 | 29 | 1.41E−09 | 1 |
| GO:0009605 | response to external stimulus | 1977 | 169 | 23 | 1.55E−09 | 1 |
| GO:0048522 | positive regulation of cellular process | 4633 | 326 | 60 | 1.63E−09 | 1 |
| GO:0050790 | regulation of catalytic activity | 2027 | 171 | 24 | 3.27E−09 | 1 |
| GO:0010033 | response to organic substance | 2769 | 216 | 37 | 5.32E−09 | 1 |
| GO:0051179 | localization | 5807 | 386 | 89 | 1.23E−08 | 1 |
| GO:0050817 | coagulation | 299 | 45 | 7 | 2.98E−08 | 1 |
| GO:0042221 | response to chemical | 3714 | 268 | 52 | 3.46E−08 | 1 |
| GO:0007599 | hemostasis | 301 | 45 | 7 | 3.61E−08 | 1 |
| GO:0006954 | inflammatory response | 616 | 71 | 5 | 3.81E−08 | 1 |
| GO:0071900 | regulation of protein serine/threonine kinase activity | 453 | 58 | 3 | 3.85E−08 | 1 |
| GO:0043549 | regulation of kinase activity | 765 | 82 | 4 | 4.63E−08 | 1 |
| GO:0050789 | regulation of biological process | 9629 | 577 | 124 | 5.25E−08 | 1 |
| GO:1902531 | regulation of intracellular signal transduction | 1644 | 142 | 15 | 5.25E−08 | 1 |
| GO:0007596 | blood coagulation | 296 | 44 | 7 | 6.48E−08 | 1 |
| GO:1990266 | neutrophil migration | 80 | 21 | 3 | 1.44E−07 | 1 |
| GO:0002429 | immune response-activating cell surface receptor signaling | 282 | 42 | 1 | 1.54E−07 | 1 |
| | pathway | | | | | |
| GO:0033674 | positive regulation of kinase activity | 508 | 61 | 2 | 1.54E−07 | 1 |
| GO:0031347 | regulation of defense response | 625 | 70 | 2 | 1.63E−07 | 1 |
| GO:0007166 | cell surface receptor signaling pathway | 2548 | 196 | 44 | 1.91E−07 | 0.908411304 |
| GO:0045859 | regulation of protein kinase activity | 696 | 75 | 4 | 2.14E−07 | 1 |
| GO:0097530 | granulocyte migration | 99 | 23 | 3 | 2.69E−07 | 1 |
| GO:0034097 | response to cytokine | 979 | 95 | 13 | 2.71E−07 | 1 |
| GO:0002768 | immune response-regulating cell surface receptor signaling | 311 | 44 | 2 | 2.87E−07 | 1 |
| | pathway | | | | | |
| GO:0045088 | regulation of innate immune response | 335 | 46 | 0 | 3.13E−07 | 1 |
| GO:0071345 | cellular response to cytokine stimulus | 900 | 89 | 11 | 3.74E−07 | 1 |
| GO:0097529 | myeloid leukocyte migration | 156 | 29 | 4 | 4.42E−07 | 1 |
| GO:0065009 | regulation of molecular function | 2746 | 206 | 37 | 4.67E−07 | 1 |
| GO:0051338 | regulation of transferase activity | 863 | 86 | 5 | 4.88E−07 | 1 |
| GO:0043405 | regulation of MAP kinase activity | 306 | 43 | 2 | 5.15E−07 | 1 |
| GO:0071902 | positive regulation of protein serine/threonine kinase | 296 | 42 | 1 | 5.88E−07 | 1 |
| | activity | | | | | |
| GO:0009607 | response to biotic stimulus | 775 | 79 | 4 | 8.36E−07 | 1 |
| GO:0032879 | regulation of localization | 2382 | 183 | 32 | 8.36E−07 | 1 |
| GO:0045860 | positive regulation of protein kinase activity | 469 | 56 | 2 | 8.36E−07 | 1 |
| GO:0009967 | positive regulation of signal transduction | 1414 | 122 | 17 | 1.12E−06 | 1 |
| GO:0051347 | positive regulation of transferase activity | 578 | 64 | 3 | 1.22E−06 | 1 |
| GO:0019221 | cytokine-mediated signaling pathway | 619 | 67 | 9 | 1.30E−06 | 1 |
| GO:0031349 | positive regulation of defense response | 379 | 48 | 1 | 1.72E−06 | 1 |
| GO:1902533 | positive regulation of intracellular signal transduction | 934 | 89 | 8 | 1.99E−06 | 1 |
| GO:0009966 | regulation of signal transduction | 2771 | 204 | 36 | 2.46E−06 | 1 |
| GO:0071621 | granulocyte chemotaxis | 86 | 20 | 2 | 2.75E−06 | 1 |
| GO:0030593 | neutrophil chemotaxis | 70 | 18 | 2 | 2.76E−06 | 1 |
| GO:0023056 | positive regulation of signaling | 1565 | 130 | 20 | 3.03E−06 | 1 |
| GO:1901700 | response to oxygen-containing compound | 1398 | 119 | 24 | 3.59E−06 | 1 |
| GO:0038093 | Fc receptor signaling pathway | 124 | 24 | 0 | 4.29E−06 | 1 |
| GO:0010942 | positive regulation of cell death | 615 | 65 | 5 | 5.18E−06 | 1 |
| GO:0023051 | regulation of signaling | 3129 | 223 | 39 | 5.75E−06 | 1 |
| GO:0010647 | positive regulation of cell communication | 1558 | 128 | 20 | 7.38E−06 | 1 |
| GO:0051707 | response to other organism | 747 | 74 | 4 | 7.77E−06 | 1 |
| GO:0043207 | response to external biotic stimulus | 748 | 74 | 4 | 8.13E−06 | 1 |
| GO:0008219 | cell death | 1947 | 152 | 18 | 8.16E−06 | 1 |
| GO:0043068 | positive regulation of programmed cell death | 569 | 61 | 5 | 8.16E−06 | 1 |
| GO:0002218 | activation of innate immune response | 233 | 34 | 0 | 8.37E−06 | 1 |
| GO:0051247 | positive regulation of protein metabolic process | 1469 | 122 | 16 | 8.56E−06 | 1 |
| GO:0010646 | regulation of cell communication | 3097 | 220 | 39 | 9.07E−06 | 1 |
| GO:0042110 | T cell activation | 414 | 49 | 4 | 9.07E−06 | 1 |
| GO:0040017 | positive regulation of locomotion | 481 | 54 | 8 | 1.08E−05 | 1 |
| GO:0043406 | positive regulation of MAP kinase activity | 237 | 34 | 1 | 1.23E−05 | 1 |
| GO:0009987 | cellular process | 13404 | 738 | 179 | 1.26E−05 | 1 |
| GO:0045089 | positive regulation of innate immune response | 284 | 38 | 0 | 1.31E−05 | 1 |
| GO:0043065 | positive regulation of apoptotic process | 565 | 60 | 5 | 1.38E−05 | 1 |
| GO:0040012 | regulation of locomotion | 848 | 80 | 13 | 1.54E−05 | 1 |
| GO:0012501 | programmed cell death | 1824 | 143 | 17 | 1.62E−05 | 1 |
| GO:0030335 | positive regulation of cell migration | 437 | 50 | 8 | 1.81E−05 | 1 |
| GO:0001816 | cytokine production | 653 | 66 | 4 | 1.87E−05 | 1 |
| GO:0006915 | apoptotic process | 1721 | 136 | 15 | 2.24E−05 | 1 |
| GO:0000186 | activation of MAPKK activity | 49 | 14 | 0 | 2.35E−05 | 1 |
| GO:1901698 | response to nitrogen compound | 931 | 85 | 12 | 2.35E−05 | 1 |
| GO:0033554 | cellular response to stress | 1775 | 139 | 14 | 2.63E−05 | 1 |
| GO:0032147 | activation of protein kinase activity | 294 | 38 | 1 | 3.02E−05 | 1 |
| GO:0050878 | regulation of body fluid levels | 433 | 49 | 8 | 3.25E−05 | 1 |
| GO:0051246 | regulation of protein metabolic process | 2429 | 178 | 24 | 3.25E−05 | 1 |
| GO:0002758 | innate immune response-activating signal transduction | 214 | 31 | 0 | 3.39E−05 | 1 |
| GO:0044248 | cellular catabolic process | 2063 | 156 | 13 | 3.41E−05 | 1 |
| GO:0051272 | positive regulation of cellular component movement | 462 | 51 | 8 | 3.82E−05 | 1 |
| GO:0051270 | regulation of cellular component movement | 855 | 79 | 14 | 3.97E−05 | 1 |
| GO:0030595 | leukocyte chemotaxis | 172 | 27 | 4 | 4.04E−05 | 1 |
| GO:2000147 | positive regulation of cell motility | 450 | 50 | 8 | 4.04E−05 | 1 |
| GO:0051092 | positive regulation of NF-kappaB transcription factor | 141 | 24 | 0 | 4.12E−05 | 1 |
| | activity | | | | | |
| GO:0016477 | cell migration | 1236 | 104 | 27 | 4.44E−05 | 0.374640189 |
| GO:0042327 | positive regulation of phosphorylation | 892 | 81 | 7 | 5.46E−05 | 1 |
| GO:0050900 | leukocyte migration | 365 | 43 | 11 | 5.85E−05 | 0.391246998 |
| GO:0046649 | lymphocyte activation | 592 | 60 | 7 | 5.94E−05 | 1 |
| GO:0038094 | Fc-gamma receptor signaling pathway | 78 | 17 | 0 | 6.74E−05 | 1 |
| GO:0040011 | locomotion | 1566 | 124 | 33 | 7.17E−05 | 0.318994972 |
| GO:0032268 | regulation of cellular protein metabolic process | 2258 | 166 | 19 | 7.19E−05 | 1 |
| GO:0030168 | platelet activation | 146 | 24 | 3 | 7.58E−05 | 1 |
| GO:0009056 | catabolic process | 2322 | 169 | 21 | 0.000102197 | 1 |
| GO:0010562 | positive regulation of phosphorus metabolic process | 952 | 84 | 7 | 0.000102197 | 1 |
| GO:0045937 | positive regulation of phosphate metabolic process | 952 | 84 | 7 | 0.000102197 | 1 |
| GO:1901701 | cellular response to oxygen-containing compound | 984 | 86 | 22 | 0.000109264 | 0.469765814 |
| GO:0030334 | regulation of cell migration | 733 | 69 | 12 | 0.00011376 | 1 |
| GO:1901699 | cellular response to nitrogen compound | 563 | 57 | 12 | 0.000115015 | 1 |
| GO:0043408 | regulation of MAPK cascade | 648 | 63 | 6 | 0.000117993 | 1 |
| GO:0010243 | response to organonitrogen compound | 868 | 78 | 12 | 0.000126448 | 1 |
| GO:2000145 | regulation of cell motility | 780 | 72 | 13 | 0.000129242 | 1 |
| GO:0048870 | cell motility | 1366 | 110 | 28 | 0.000148918 | 0.546976326 |
| GO:0051674 | localization of cell | 1366 | 110 | 28 | 0.000148918 | 0.546976326 |
| GO:0071417 | cellular response to organonitrogen compound | 513 | 53 | 12 | 0.000148943 | 0.802988607 |
| GO:0051345 | positive regulation of hydrolase activity | 684 | 65 | 12 | 0.000166794 | 1 |
| GO:0098657 | import into cell | 757 | 70 | 16 | 0.000169245 | 0.853144703 |
| GO:0032880 | regulation of protein localization | 891 | 79 | 5 | 0.000173297 | 1 |
| GO:0032101 | regulation of response to external stimulus | 686 | 65 | 4 | 0.00018111 | 1 |
| GO:0001934 | positive regulation of protein phosphorylation | 849 | 76 | 7 | 0.00019432 | 1 |
| GO:0042325 | regulation of phosphorylation | 1329 | 107 | 11 | 0.000207752 | 1 |
| GO:0032270 | positive regulation of cellular protein metabolic | 1378 | 110 | 11 | 0.000215866 | 1 |
| | process | | | | | |
| GO:1901652 | response to peptide | 426 | 46 | 7 | 0.000230098 | 1 |
| GO:0051049 | regulation of transport | 1607 | 124 | 21 | 0.000238452 | 1 |
| GO:0061024 | membrane organization | 736 | 68 | 8 | 0.000239587 | 1 |
| GO:0001932 | regulation of protein phosphorylation | 1225 | 100 | 11 | 0.000255758 | 1 |
| GO:0007159 | leukocyte cell-cell adhesion | 299 | 36 | 5 | 0.000269107 | 1 |
| GO:0009306 | protein secretion | 483 | 50 | 4 | 0.000269107 | 1 |
| GO:0002687 | positive regulation of leukocyte migration | 106 | 19 | 2 | 0.000274565 | 1 |
| GO:0006468 | protein phosphorylation | 1678 | 128 | 21 | 0.000274565 | 1 |
| GO:0031098 | stress-activated protein kinase signaling cascade | 287 | 35 | 0 | 0.000274565 | 1 |
| GO:0071216 | cellular response to biotic stimulus | 203 | 28 | 0 | 0.000274689 | 1 |
| GO:0065008 | regulation of biological quality | 3483 | 233 | 50 | 0.000278675 | 1 |
| GO:0033036 | macromolecule localization | 2816 | 195 | 28 | 0.000293603 | 1 |
| GO:0043410 | positive regulation of MAPK cascade | 473 | 49 | 4 | 0.000320863 | 1 |
| GO:0007155 | cell adhesion | 1250 | 101 | 32 | 0.000336573 | 0.040286053 |
| GO:0019220 | regulation of phosphate metabolic process | 1507 | 117 | 13 | 0.000343159 | 1 |
| GO:0031667 | response to nutrient levels | 421 | 45 | 3 | 0.000362009 | 1 |
| GO:0051174 | regulation of phosphorus metabolic process | 1509 | 117 | 13 | 0.000362009 | 1 |
| GO:0006909 | phagocytosis | 230 | 30 | 2 | 0.000365939 | 1 |
| GO:0002431 | Fc receptor mediated stimulatory signaling pathway | 80 | 16 | 0 | 0.000387008 | 1 |
| GO:0022610 | biological adhesion | 1257 | 101 | 32 | 0.000417147 | 0.043138 |
| GO:0050663 | cytokine secretion | 185 | 26 | 1 | 0.000425866 | 1 |
| GO:0023014 | signal transduction by protein phosphorylation | 797 | 71 | 10 | 0.000463812 | 1 |
| GO:0015031 | protein transport | 1831 | 136 | 17 | 0.000473998 | 1 |
| GO:0071496 | cellular response to external stimulus | 308 | 36 | 4 | 0.000486676 | 1 |
| GO:0060326 | cell chemotaxis | 234 | 30 | 5 | 0.0005037 | 1 |
| GO:0045184 | establishment of protein localization | 1934 | 142 | 19 | 0.000506381 | 1 |
| GO:0030155 | regulation of cell adhesion | 609 | 58 | 8 | 0.000509038 | 1 |
| GO:0045932 | negative regulation of muscle contraction | 20 | 8 | 0 | 0.000531728 | 1 |
| GO:0071702 | organic substance transport | 2515 | 176 | 27 | 0.000535431 | 1 |
| GO:0006914 | autophagy | 443 | 46 | 0 | 0.000565442 | 1 |
| GO:0061919 | process utilizing autophagic mechanism | 443 | 46 | 0 | 0.000565442 | 1 |
| GO:0051336 | regulation of hydrolase activity | 1127 | 92 | 16 | 0.000599955 | 1 |
| GO:0006897 | endocytosis | 645 | 60 | 15 | 0.000695764 | 0.678522254 |
| GO:0071260 | cellular response to mechanical stimulus | 75 | 15 | 2 | 0.000730688 | 1 |
| GO:0042886 | amide transport | 1885 | 138 | 19 | 0.000791641 | 1 |
| GO:0032612 | interleukin-1 production | 76 | 15 | 0 | 0.000859296 | 1 |
| GO:0006796 | phosphate-containing compound metabolic process | 2886 | 196 | 26 | 0.000875096 | 1 |
| GO:0032103 | positive regulation of response to external stimulus | 254 | 31 | 3 | 0.000898772 | 1 |
| GO:0002790 | peptide secretion | 508 | 50 | 6 | 0.000925557 | 1 |
| GO:0031401 | positive regulation of protein modification process | 1046 | 86 | 7 | 0.000925557 | 1 |
| GO:0010952 | positive regulation of peptidase activity | 171 | 24 | 1 | 0.000939318 | 1 |
| GO:0015833 | peptide transport | 1859 | 136 | 19 | 0.000941858 | 1 |
| GO:0009991 | response to extracellular stimulus | 453 | 46 | 3 | 0.000954405 | 1 |
| GO:1901653 | cellular response to peptide | 319 | 36 | 5 | 0.000970827 | 1 |
| GO:0034134 | toll-like receptor 2 signaling pathway | 16 | 7 | 0 | 0.001002353 | 1 |
| GO:1901564 | organonitrogen compound metabolic process | 6040 | 367 | 60 | 0.00113986 | 1 |
| GO:0009894 | regulation of catabolic process | 823 | 71 | 4 | 0.001233752 | 1 |
| GO:0000165 | MAPK cascade | 779 | 68 | 10 | 0.001302406 | 1 |
| GO:0002237 | response to molecule of bacterial origin | 299 | 34 | 2 | 0.001448175 | 1 |
| GO:0010950 | positive regulation of endopeptidase activity | 153 | 22 | 1 | 0.001467807 | 1 |
| GO:0016310 | phosphorylation | 2046 | 146 | 21 | 0.001473433 | 1 |
| GO:0006793 | phosphorus metabolic process | 2913 | 196 | 26 | 0.001490737 | 1 |
| GO:0009617 | response to bacterium | 490 | 48 | 3 | 0.001514023 | 1 |
| GO:0051046 | regulation of secretion | 679 | 61 | 5 | 0.001556577 | 1 |
| GO:0043067 | regulation of programmed cell death | 1367 | 105 | 13 | 0.001586821 | 1 |
| GO:2000116 | regulation of cysteine-type endopeptidase activity | 213 | 27 | 1 | 0.001630423 | 1 |
| GO:0030162 | regulation of proteolysis | 651 | 59 | 3 | 0.001650328 | 1 |
| GO:0051091 | positive regulation of DNA-binding transcription | 238 | 29 | 0 | 0.001674787 | 1 |
| | factor activity | | | | | |
| GO:0042981 | regulation of apoptotic process | 1354 | 104 | 13 | 0.001724948 | 1 |
| GO:0002286 | T cell activation involved in immune response | 81 | 15 | 1 | 0.001731296 | 1 |
| GO:0070498 | interleukin-1-mediated signaling pathway | 54 | 12 | 0 | 0.001861726 | 1 |
| GO:0002685 | regulation of leukocyte migration | 156 | 22 | 2 | 0.001893805 | 1 |
| GO:0080135 | regulation of cellular response to stress | 625 | 57 | 3 | 0.001893805 | 1 |
| GO:0008150 | biological_process | 14638 | 780 | 198 | 0.001926983 | 1 |
| GO:0051704 | multi-organism process | 2076 | 147 | 14 | 0.001926983 | 1 |
| GO:0031399 | regulation of protein modification process | 1590 | 118 | 12 | 0.002015929 | 1 |
| GO:0010941 | regulation of cell death | 1477 | 111 | 13 | 0.00215311 | 1 |
| GO:1903037 | regulation of leukocyte cell-cell adhesion | 267 | 31 | 3 | 0.00215311 | 1 |
| GO:2001056 | positive regulation of cysteine-type endopeptidase | 135 | 20 | 1 | 0.002158532 | 1 |
| | activity | | | | | |
| GO:0050867 | positive regulation of cell activation | 294 | 33 | 4 | 0.002322146 | 1 |
| GO:0008104 | protein localization | 2517 | 172 | 25 | 0.002381633 | 1 |
| GO:0050701 | interleukin-1 secretion | 47 | 11 | 0 | 0.002404685 | 1 |
| GO:0002688 | regulation of leukocyte chemotaxis | 94 | 16 | 2 | 0.002570483 | 1 |
| GO:0002223 | stimulatory C-type lectin receptor signaling pathway | 56 | 12 | 0 | 0.00259536 | 1 |
| GO:0032611 | interleukin-1 beta production | 65 | 13 | 0 | 0.00259536 | 1 |
| GO:0001817 | regulation of cytokine production | 589 | 54 | 4 | 0.002597853 | 1 |
| GO:0002694 | regulation of leukocyte activation | 446 | 44 | 4 | 0.002664683 | 1 |
| GO:0002696 | positive regulation of leukocyte activation | 284 | 32 | 4 | 0.002776889 | 1 |
| GO:0002433 | immune response-regulating cell surface receptor | 75 | 14 | 0 | 0.002791965 | 1 |
| | signaling pathway involved in phagocytosis | | | | | |
| GO:0038096 | Fc-gamma receptor signaling pathway involved in | 75 | 14 | 0 | 0.002791965 | 1 |
| | phagocytosis | | | | | |
| GO:0051770 | positive regulation of nitric-oxide synthase | 13 | 6 | 0 | 0.002791965 | 1 |
| | biosynthetic process | | | | | |
| GO:0050863 | regulation of T cell activation | 285 | 32 | 3 | 0.002907125 | 1 |
| GO:0098542 | defense response to other organism | 365 | 38 | 0 | 0.002907125 | 1 |
| GO:0048519 | negative regulation of biological process | 4605 | 287 | 58 | 0.003060718 | 1 |
| GO:0010604 | positive regulation of macromolecule metabolic | 2902 | 193 | 37 | 0.003155111 | 1 |
| | process | | | | | |
| GO:0045862 | positive regulation of proteolysis | 326 | 35 | 2 | 0.003155111 | 1 |
| GO:0051050 | positive regulation of transport | 851 | 71 | 13 | 0.003155111 | 1 |
| GO:0050851 | antigen receptor-mediated signaling pathway | 174 | 23 | 1 | 0.003171354 | 1 |
| GO:0031329 | regulation of cellular catabolic process | 715 | 62 | 0 | 0.003331165 | 1 |
| GO:0050865 | regulation of cell activation | 480 | 46 | 4 | 0.00340888 | 1 |
| GO:0052548 | regulation of endopeptidase activity | 341 | 36 | 2 | 0.003414219 | 1 |
| GO:0051223 | regulation of protein transport | 612 | 55 | 3 | 0.00355489 | 1 |
| GO:0001819 | positive regulation of cytokine production | 383 | 39 | 4 | 0.003612705 | 1 |
| GO:0051222 | positive regulation of protein transport | 370 | 38 | 2 | 0.003761834 | 1 |
| GO:0002220 | innate immune response activating cell surface | 59 | 12 | 0 | 0.004134873 | 1 |
| | receptor signaling pathway | | | | | |
| GO:0038095 | Fc-epsilon receptor signaling pathway | 59 | 12 | 0 | 0.004134873 | 1 |
| GO:1903530 | regulation of secretion by cell | 631 | 56 | 4 | 0.004194911 | 1 |
| GO:0072672 | neutrophil extravasation | 9 | 5 | 1 | 0.004323416 | 1 |
| GO:0051240 | positive regulation of multicellular organismal | 1506 | 111 | 22 | 0.004358097 | 1 |
| | process | | | | | |
| GO:0050702 | interleukin-1 beta secretion | 42 | 10 | 0 | 0.004383898 | 1 |
| GO:0002697 | regulation of immune effector process | 348 | 36 | 3 | 0.00505513 | 1 |
| GO:0002221 | pattern recognition receptor signaling pathway | 168 | 22 | 0 | 0.005171898 | 1 |
| GO:0009893 | positive regulation of metabolic process | 3126 | 204 | 38 | 0.005271532 | 1 |
| GO:0051239 | regulation of multicellular organismal process | 2648 | 177 | 41 | 0.005403955 | 1 |
| GO:0002755 | MyD88-dependent toll-like receptor signaling pathway | 35 | 9 | 0 | 0.005460604 | 1 |
| GO:1904951 | positive regulation of establishment of protein | 405 | 40 | 3 | 0.005460604 | 1 |
| | localization | | | | | |
| GO:0002250 | adaptive immune response | 350 | 36 | 4 | 0.005571526 | 1 |
| GO:0016050 | vesicle organization | 296 | 32 | 1 | 0.005571526 | 1 |
| GO:0045785 | positive regulation of cell adhesion | 364 | 37 | 4 | 0.005594484 | 1 |
| GO:0052547 | regulation of peptidase activity | 364 | 37 | 2 | 0.005594484 | 1 |
| GO:0070201 | regulation of establishment of protein localization | 654 | 57 | 4 | 0.005626218 | 1 |
| GO:0006928 | movement of cell or subcelIular component | 1787 | 127 | 41 | 0.005781787 | 0.054108794 |
| GO:0002367 | cytokine production involved in immune response | 91 | 15 | 1 | 0.005828657 | 1 |
| GO:0071219 | cellular response to molecule of bacterial origin | 182 | 23 | 0 | 0.005892329 | 1 |
| GO:0061041 | regulation of wound healing | 113 | 17 | 0 | 0.006255149 | 1 |
| GO:0032757 | positive regulation of interleukin-8 production | 44 | 10 | 0 | 0.006351224 | 1 |
| GO:0071675 | regulation of mononuclear cell migration | 36 | 9 | 1 | 0.006679936 | 1 |
| GO:0051403 | stress-activated MAPK cascade | 260 | 29 | 0 | 0.006850318 | 1 |
| GO:0031323 | regulation of cellular metabolic process | 5315 | 322 | 54 | 0.006855097 | 1 |
| GO:0050707 | regulation of cytokine secretion | 160 | 21 | 1 | 0.006933616 | 1 |
| GO:0032496 | response to lipopolysaccharide | 287 | 31 | 2 | 0.007057627 | 1 |
| GO:0071705 | nitrogen compound transport | 2158 | 148 | 22 | 0.007275744 | 1 |
| GO:0010256 | endomembrane system organization | 384 | 38 | 3 | 0.00755502 | 1 |
| GO:0001776 | leukocyte homeostasis | 83 | 14 | 3 | 0.007636898 | 1 |
| GO:0002690 | positive regulation of leukocyte chemotaxis | 73 | 13 | 2 | 0.007690198 | 1 |
| GO:0071674 | mononuclear cell migration | 54 | 11 | 1 | 0.007715492 | 1 |
| GO:0098609 | cell-cell adhesion | 724 | 61 | 10 | 0.007721864 | 1 |
| GO:0000187 | activation of MAPK activity | 138 | 19 | 0 | 0.007734155 | 1 |
| GO:1903523 | negative regulation of blood circulation | 29 | 8 | 0 | 0.007734155 | 1 |
| GO:0050793 | regulation of developmental process | 2215 | 151 | 40 | 0.007916914 | 0.781661155 |
| GO:0071622 | regulation of granulocyte chemotaxis | 37 | 9 | 0 | 0.008028146 | 1 |
| GO:0090087 | regulation of peptide transport | 635 | 55 | 4 | 0.008354822 | 1 |
| GO:0051130 | positive regulation of cellular component organization | 1123 | 86 | 17 | 0.00860929 | 1 |
| GO:0031325 | positive regulation of cellular metabolic process | 2873 | 188 | 31 | 0.00899248 | 1 |
| GO:0001525 | angiogenesis | 445 | 42 | 6 | 0.0090508 | 1 |
| GO:0043087 | regulation of GTPase activity | 431 | 41 | 10 | 0.009152495 | 0.989075954 |
| GO:0071347 | cellular response to interleukin-1 | 106 | 16 | 0 | 0.009152495 | 1 |
| GO:1903827 | regulation of cellular protein localization | 489 | 45 | 3 | 0.009195558 | 1 |
| GO:0016236 | macroautophagy | 266 | 29 | 0 | 0.00959439 | 1 |
| GO:1902622 | regulation of neutrophil migration | 30 | 8 | 0 | 0.009698552 | 1 |
| GO:0051249 | regulation of lymphocyte activation | 376 | 37 | 4 | 0.009906385 | 1 |
| GO:0050715 | positive regulation of cytokine secretion | 108 | 16 | 1 | 0.01128157 | 1 |
| GO:0009743 | response to carbohydrate | 204 | 24 | 3 | 0.011633007 | 1 |
| GO:0070302 | regulation of stress-activated protein kinase | 217 | 25 | 0 | 0.011808339 | 1 |
| | signaling cascade | | | | | |
| GO:0048013 | ephrin receptor signaling pathway | 77 | 13 | 1 | 0.012690739 | 1 |
| GO:0007169 | transmembrane receptor protein tyrosine kinase | 616 | 53 | 11 | 0.012718363 | 1 |
| | signaling pathway | | | | | |
| GO:0006464 | cellular protein modification process | 3634 | 229 | 39 | 0.012821559 | 1 |
| GO:0036211 | protein modification process | 3634 | 229 | 39 | 0.012821559 | 1 |
| GO:0043281 | regulation of cysteine-type endopeptidase activity | 193 | 23 | 0 | 0.12949805 | 1 |
| | involved in apoptotic process | | | | | |
| GO:0051767 | nitric-oxide synthase biosynthetic process | 17 | 6 | 0 | 0.013035488 | 1 |
| GO:0051769 | regulation of nitric-oxide synthase biosynthetic | 17 | 6 | 0 | 0.013035488 | 1 |
| | process | | | | | |
| GO:0070340 | detection of bacterial lipopeptide | 3 | 3 | 0 | 0.01315179 | 1 |
| GO:0002460 | adaptive immune response based on somatic | 233 | 26 | 4 | 0.014276364 | 1 |
| | recombination of immune receptors built from | | | | | |
| | immunoglobin superfamily domains | | | | | |
| GO:0002576 | platelet degranulation | 122 | 17 | 1 | 0.014503148 | 1 |
| GO:0050727 | regulation of inflammatory response | 314 | 32 | 2 | 0.014538693 | 1 |
| GO:0002224 | toll-like receptor signaling pathway | 123 | 17 | 0 | 0.01594757 | 1 |
| GO:0043123 | positive regulation of I-kappaB kinase/NF-kappaB | 171 | 21 | 1 | 0.016024765 | 1 |
| | signaling | | | | | |
| GO:0045597 | positive regulation of cell differentiation | 858 | 68 | 15 | 0.017105336 | 1 |
| GO:0043547 | positive regulation of GTPase activity | 359 | 35 | 9 | 0.017159123 | 0.870477156 |
| GO:1903034 | regulation of response to wounding | 136 | 18 | 0 | 0.017874522 | 1 |
| GO:0051128 | regulation of cellular component organization | 2275 | 152 | 32 | 0.017920515 | 1 |
| GO:0030099 | myeloid cell differentiation | 360 | 35 | 5 | 0.017939107 | 1 |
| GO:0051173 | positive regulation of nitrogen compound metabolic | 2771 | 180 | 34 | 0.01800847 | 1 |
| | process | | | | | |
| GO:0051094 | positive regulation of developmental process | 1201 | 89 | 21 | 0.018331106 | 1 |
| GO:0032677 | regulation of interleukin-8 production | 60 | 11 | 0 | 0.018411813 | 1 |
| GO:0070997 | neuron death | 305 | 31 | 1 | 0.018600627 | 1 |
| GO:0098543 | detection of other organism | 12 | 5 | 0 | 0.018875179 | 1 |
| GO:0051048 | negative regulation of secretion | 186 | 22 | 2 | 0.01891199 | 1 |
| GO:0009057 | macromolecule catabolic process | 1269 | 93 | 10 | 0.018999165 | 1 |
| GO:0045916 | negative regulation of complement activation | 7 | 4 | 0 | 0.01930961 | 1 |
| GO:0051056 | regulation of small GTPase mediated signal | 306 | 31 | 6 | 0.01930961 | 1 |
| | transduction | | | | | |
| GO:0071476 | cellular hypotonic response | 7 | 4 | 0 | 0.01930961 | 1 |
| GO:2000258 | negative regulation of protein activation cascade | 7 | 4 | 0 | 0.01930961 | 1 |
| GO:0031663 | lipopolysaccharide-mediated signaling pathway | 51 | 10 | 0 | 0.019532324 | 1 |
| GO:0045123 | cellular extravasation | 51 | 10 | 3 | 0.019532324 | 0.669150896 |
| GO:0044267 | cellular protein metabolic process | 4551 | 277 | 47 | 0.019796909 | 1 |
| GO:1901575 | organic substance catabolic process | 1934 | 132 | 16 | 0.020007773 | 1 |
| GO:0006937 | regulation of muscle contraction | 138 | 18 | 1 | 0.020374992 | 1 |
| GO:0043434 | response to peptide hormone | 364 | 35 | 6 | 0.021070827 | 1 |
| GO:0050921 | positive regulation of chemotaxis | 115 | 16 | 2 | 0.02118087 | 1 |
| GO:0048523 | negative regulation of cellular process | 4113 | 253 | 52 | 0.021899591 | 1 |
| GO:0051051 | negative regulation of transport | 408 | 38 | 6 | 0.021899591 | 1 |
| GO:0010661 | positive regulation of muscle cell apoptotic process | 26 | 7 | 1 | 0.02192505 | 1 |
| GO:0019538 | protein metabolic process | 5067 | 304 | 56 | 0.022521548 | 1 |
| GO:0032872 | regulation of stress-activated MAPK cascade | 215 | 24 | 0 | 0.022830476 | 1 |
| GO:0035743 | CD4-positive, alpha-beta T cell cytokine production | 19 | 6 | 0 | 0.023474679 | 1 |
| GO:0033673 | negative regulation of kinase activity | 216 | 24 | 2 | 0.024339447 | 1 |
| GO:0009896 | positive regulation of catabolic process | 382 | 36 | 4 | 0.024678993 | 1 |
| GO:0080090 | regulation of primary metabolic process | 5208 | 311 | 55 | 0.025290196 | 1 |
| GO:0002718 | regulation of cytokine production involved in immune | 73 | 12 | 1 | 0.025671832 | 1 |
| | response | | | | | |
| GO:0070482 | response to oxygen levels | 312 | 31 | 3 | 0.025671832 | 1 |
| GO:0042594 | response to starvation | 166 | 20 | 0 | 0.026248102 | 1 |
| GO:0043086 | negative regulation of catalytic activity | 669 | 55 | 6 | 0.026248102 | 1 |
| GO:0051348 | negative regulation of transferase activity | 244 | 26 | 2 | 0.026248102 | 1 |
| GO:0060759 | regulation of response to cytokine stimulus | 154 | 19 | 0 | 0.027030672 | 1 |
| GO:0050714 | positive regulation of protein secretion | 218 | 24 | 1 | 0.027033414 | 1 |
| GO:0090022 | regulation of neutrophil chemotaxis | 27 | 7 | 0 | 0.027033414 | 1 |
| GO:1904646 | cellular response to amyloid-beta | 27 | 7 | 1 | 0.027033414 | 1 |
| GO:0008283 | cell proliferation | 1761 | 121 | 28 | 0.027213333 | 1 |
| GO:0022407 | regulation of cell-cell adhesion | 356 | 34 | 4 | 0.027337401 | 1 |
| GO:0006508 | proteolysis | 1576 | 110 | 15 | 0.02922209 | 1 |
| GO:0072593 | reactive oxygen species metabolic process | 246 | 26 | 3 | 0.02922209 | 1 |
| GO:0043280 | positive regulation of cysteine-type endopeptidase | 119 | 16 | 0 | 0.029336158 | 1 |
| | activity involved in apoptotic process | | | | | |
| GO:0002526 | acute inflammatory response | 131 | 17 | 0 | 0.029626108 | 1 |
| GO:0032868 | response to insulin | 233 | 25 | 4 | 0.029731739 | 1 |
| GO:0050708 | regulation of protein secretion | 387 | 36 | 2 | 0.030095053 | 1 |
| GO:0044247 | cellular polysaccharide catabolic process | 20 | 6 | 0 | 0.0301116 | 1 |
| GO:0090025 | regulation of monocyte chemotaxis | 20 | 6 | 1 | 0.0301116 | 1 |
| GO:0007160 | cell-matrix adhesion | 207 | 23 | 5 | 0.03037299 | 1 |
| GO:0006935 | chemotaxis | 537 | 46 | 11 | 0.031747962 | 1 |
| GO:0051251 | positive regulation of lymphocyte activation | 248 | 26 | 4 | 0.032193807 | 1 |
| GO:0035744 | T-helper 1 cell cytokine production | 8 | 4 | 0 | 0.032836459 | 1 |
| GO:0007264 | small GTPase mediated signal transduction | 523 | 45 | 10 | 0.032933819 | 1 |
| GO:0019222 | regulation of metabolic process | 5763 | 339 | 59 | 0.032947746 | 1 |
| GO:0042330 | taxis | 539 | 46 | 11 | 0.033892587 | 1 |
| GO:0050852 | T cell receptor signaling pathway | 133 | 17 | 1 | 0.034161479 | 1 |
| GO:0051090 | regulation of DNA-binding transcription factor | 377 | 35 | 2 | 0.036224025 | 1 |
| | activity | | | | | |
| GO:0050920 | regulation of chemotaxis | 184 | 21 | 2 | 0.036574118 | 1 |
| GO:0009267 | cellular response to starvation | 134 | 17 | 0 | 0.0368303 | 1 |
| GO:0032637 | interleukin-8 production | 66 | 11 | 0 | 0.0368303 | 1 |
| GO:0061025 | membrane fusion | 134 | 17 | 0 | 0.0368303 | 1 |
| GO:1903901 | negative regulation of viral life cycle | 66 | 11 | 0 | 0.0368303 | 1 |
| GO:0090026 | positive regulation of monocyte chemotaxis | 14 | 5 | 1 | 0.037038122 | 1 |
| GO:0006469 | negative regulation of protein kinase activity | 198 | 22 | 2 | 0.03826875 | 1 |
| GO:0006979 | response to oxidative stress | 408 | 37 | 3 | 0.03826875 | 1 |
| GO:0036473 | cell death in response to oxidative stress | 77 | 12 | 0 | 0.03826875 | 1 |
| GO:0048534 | hematopoietic or lymphoid organ development | 777 | 61 | 11 | 0.03826875 | 1 |
| GO:0050818 | regulation of coagulation | 77 | 12 | 0 | 0.03826875 | 1 |
| GO:1903039 | positive regulation of leukocyte cell-cell adhesion | 198 | 22 | 3 | 0.03826875 | 1 |
| GO:0001782 | B cell homeostasis | 29 | 7 | 1 | 0.039601018 | 1 |
| GO:0001999 | renal response to blood flow involved in circulatory | 4 | 3 | 0 | 0.039601018 | 1 |
| | renin-angiotensin regulation of systemic arterial | | | | | |
| | blood pressure | | | | | |
| GO:0002001 | renin secretion into blood stream | 4 | 3 | 0 | 0.039601018 | 1 |
| GO:0042494 | detection of bacterial lipoprotein | 4 | 3 | 0 | 0.039601018 | 1 |
| GO:1903223 | positive regulation of oxidative stress-induced | 4 | 3 | 0 | 0.039601018 | 1 |
| | neuron death | | | | | |
| GO:0002720 | positive regulation of cytokine production involved | 47 | 9 | 1 | 0.040286053 | 1 |
| | in immune response | | | | | |
| GO:0070265 | necrotic cell death | 47 | 9 | 0 | 0.040286053 | 1 |
| GO:0030888 | regulation of B cell proliferation | 57 | 10 | 1 | 0.041271788 | 1 |
| GO:0032729 | positive regulation of interferon-gamma production | 57 | 10 | 1 | 0.041271788 | 1 |
| GO:0070527 | platelet aggregation | 57 | 10 | 0 | 0.041271788 | 1 |
| GO:0002822 | regulation of adaptive immune response based on | 136 | 17 | 3 | 0.041344145 | 1 |
| | somatic recombination of immune receptors built | | | | | |
| | from immunoglobin superfamily domains | | | | | |
| GO:0097300 | programmed necrotic cell death | 38 | 8 | 0 | 0.041379424 | 1 |
| GO:2000377 | regulation of reactive oxygen species metabolic | 161 | 19 | 0 | 0.041379424 | 1 |
| | process | | | | | |
| GO:0006629 | lipid metabolic process | 1274 | 91 | 10 | 0.041845478 | 1 |
| GO:0043112 | receptor metabolic process | 174 | 20 | 5 | 0.041928718 | 1 |
| GO:0005976 | polysaccharide metabolic process | 101 | 14 | 1 | 0.043569205 | 1 |
| GO:0043122 | regulation of I-kappaB kinase/NF-kappaB signaling | 214 | 23 | 1 | 0.043569205 | 1 |
| GO:0022603 | regulation of anatomical structure morphogenesis | 912 | 69 | 18 | 0.043665082 | 1 |
| GO:0043412 | macromolecule modification | 3819 | 234 | 41 | 0.043813518 | 1 |
| GO:0071453 | cellular response to oxygen levels | 162 | 19 | 1 | 0.043846908 | 1 |
| GO:0000271 | polysaccharide biosynthetic process | 68 | 11 | 0 | 0.044145176 | 1 |
| GO:0050870 | positive regulation of T cell activation | 188 | 21 | 3 | 0.044145176 | 1 |
| GO:0071222 | cellular response to lipopolysaccharide | 175 | 20 | 0 | 0.044145176 | 1 |
| GO:1901214 | regulation of neuron death | 269 | 27 | 1 | 0.044145176 | 1 |
| GO:0070555 | response to interleukin-1 | 125 | 16 | 1 | 0.044185913 | 1 |
| GO:0032269 | negative regulation of cellular protein metabolic | 898 | 68 | 8 | 0.045826564 | 1 |
| | process | | | | | |
| GO:1904645 | response to amyloid-beta | 30 | 7 | 1 | 0.045966802 | 1 |
| GO:0000272 | polysaccharide catabolic process | 22 | 6 | 0 | 0.046155493 | 1 |
| GO:0090257 | regulation of muscle system process | 202 | 22 | 2 | 0.046184075 | 1 |
| GO:0006971 | hypotonic response | 9 | 4 | 0 | 0.049853168 | 1 |
| GO:0002673 | regulation of acute inflammatory response | 80 | 12 | 0 | 0.049885523 | 1 |
| GO:0051047 | positive regulation of secretion | 357 | 33 | 3 | 0.049885523 | 1 |
| GO:0016043 | cellular component organization | 5721 | 325 | 104 | 0.246813039 | 0.031675655 |
| GO:0006607 | NLS-bearing protein import into nucleus | 27 | 3 | 4 | 1 | 0.038603188 |
| GO:0030198 | extracellular matrix organization | 321 | 15 | 18 | 1 | 0.000116024 |
| GO:0030199 | collagen fibril organization | 47 | 2 | 7 | 1 | 0.000569572 |
| GO:0043062 | extracellular structure organization | 368 | 18 | 18 | 1 | 0.000615349 |
| GO:0060351 | cartilage development involved in endochondral bone | 41 | 1 | 5 | 1 | 0.02192505 |
| | morphogenesis | | | | | |
| GO:0061138 | morphogenesis of a branching epithelium | 166 | 9 | 9 | 1 | 0.039793054 |
| GO:0061448 | connective tissue development | 233 | 6 | 13 | 1 | 0.002884637 |
| GO:0070208 | protein heterotrimerization | 12 | 0 | 3 | 1 | 0.040528723 |
| GO:0071229 | cellular response to acid chemical | 195 | 10 | 10 | 1 | 0.032559128 |
| GO:0071230 | cellular response to amino acid stimulus | 65 | 5 | 6 | 1 | 0.024461895 |

Note: The IDs are according to GeneOntology, available on the website: geneontology.org. “nGene” refers to the number of genes in the pathway. “sig_up_ngenes” and “sig_down_ngenes” represent the number of genes that were upregulated and the number of genes that were downregulated, respectively. Throughout the disclosure, “FDR” refers to false discovery rate, and an FDR of less than 0.05 indicates the change or difference in expression is significant. For example, a FDR.up of less than 0.05 indicates the upregulation of the gene

TABLE 5
Genes Common to Synovial Sublining Fibroblasts and AC3

| cluster | Geneset | ensembl | symbol | auc | pct_nonzero | pct_nonzero_other |
|---|---|---|---|---|---|---|
| SC-F1 | Fibroblast-CD34+ sublining (SC-F1) | ENSG00000187955 | COL14A1 | 0.88699058 | 0.98347107 | 0.30535427 |
| SC-F1 | Fibroblast-CD34+ sublining (SC-F1) | ENSG00000164692 | COL1A2 | 0.84468179 | 1 | 0.53280998 |
| SC-F1 | Fibroblast-CD34+ sublining (SC-F1) | ENSG00000168542 | COL3A1 | 0.82353841 | 1 | 0.53301127 |
| SC-F1 | Fibroblast-CD34+ sublining (SC-F1) | ENSG00000130635 | COL5A1 | 0.76918996 | 0.80991736 | 0.26348631 |
| SC-F1 | Fibroblast-CD34+ sublining (SC-F1) | ENSG00000105664 | COMP | 0.72267741 | 0.5268595 | 0.09178744 |
| SC-F1 | Fibroblast-CD34+ sublining (SC-F1) | ENSG00000133083 | DCLK1 | 0.78673115 | 0.70041322 | 0.16807568 |
| SC-F1 | Fibroblast-CD34+ sublining (SC-F1) | ENSG00000146648 | EGFR | 0.76375622 | 0.72933884 | 0.23007246 |
| SC-F1 | Fibroblast-CD34+ sublining (SC-F1) | ENSG00000120738 | EGR1 | 0.75381699 | 0.95041322 | 0.6507649 |
| SC-F1 | Fibroblast-CD34+ sublining (SC-F1) | ENSG00000164694 | FNDC1 | 0.7526103 | 0.63842975 | 0.15116747 |
| SC-F1 | Fibroblast-CD34+ sublining (SC-F1) | ENSG00000131386 | GALNT15 | 0.7329481 | 0.59297521 | 0.14351852 |
| SC-F1 | Fibroblast-CD34+ sublining (SC-F1) | ENSG00000168079 | SCARA5 | 0.74916074 | 0.78512397 | 0.28965378 |
| SC-F1 | Fibroblast-CD34+ sublining (SC-F1) | ENSG00000137573 | SULF1 | 0.73367922 | 0.67561983 | 0.23389694 |
| SC-F1 | Fibroblast-CD34+ sublining (SC-F1) | ENSG00000091656 | ZFHX4 | 0.77838039 | 0.71487603 | 0.2071256 |
| SC-F2 | Fibroblast-HLA-DRAhi sublining (SC-F2) | ENSG00000187955 | COL14A1 | 0.91760718 | 0.99165508 | 0.27044158 |
| SC-F2 | Fibroblast-HLA-DRAhi sublining (SC-F2) | ENSG00000084636 | COL16A1 | 0.87323477 | 0.83588317 | 0.10585252 |
| SC-F2 | Fibroblast-HLA-DRAhi sublining (SC-F2) | ENSG00000164692 | COL1A2 | 0.92139483 | 1 | 0.50961335 |
| SC-F2 | Fibroblast-HLA-DRAhi sublining (SC-F2) | ENSG00000168542 | COL3A1 | 0.93074592 | 1 | 0.50982464 |
| SC-F2 | Fibroblast-HLA-DRAhi sublining (SC-F2) | ENSG00000134871 | COL4A2 | 0.84125266 | 0.86648122 | 0.18761885 |
| SC-F2 | Fibroblast-HLA-DRAhi sublining (SC-F2) | ENSG00000130635 | COL5A1 | 0.88489277 | 0.95132128 | 0.21487429 |
| SC-F2 | Fibroblast-HLA-DRAhi sublining (SC-F2) | ENSG00000133083 | DCLK1 | 0.83131283 | 0.79972184 | 0.12655821 |
| SC-F2 | Fibroblast-HLA-DRAhi sublining (SC-F2) | ENSG00000146648 | EGFR | 0.80327353 | 0.82058414 | 0.19142193 |
| SC-F2 | Fibroblast-HLA-DRAhi sublining (SC-F2) | ENSG00000120738 | EGR1 | 0.80507545 | 0.9930459 | 0.62941052 |
| SC-F2 | Fibroblast-HLA-DRAhi sublining (SC-F2) | ENSG00000130508 | PXDN | 0.84330642 | 0.83171071 | 0.16670188 |
| SC-F2 | Fibroblast-HLA-DRAhi sublining (SC-F2) | ENSG00000166444 | ST5 | 0.83349089 | 0.81641168 | 0.15867315 |
| SC-F3 | Fibroblast-DKK3+ sublining (SC-F3) | ENSG00000187955 | COL14A1 | 0.85231287 | 0.96929825 | 0.33920368 |
| SC-F3 | Fibroblast-DKK3+ sublining (SC-F3) | ENSG00000164692 | COL1A2 | 0.90670253 | 1 | 0.55570444 |
| SC-F3 | Fibroblast-DKK3+ sublining (SC-F3) | ENSG00000168542 | COL3A1 | 0.90184724 | 1 | 0.55589587 |
| SC-F3 | Fibroblast-DKK3+ sublining (SC-F3) | ENSG00000130635 | COL5A1 | 0.91728082 | 0.98245614 | 0.28273354 |
| SC-F3 | Fibroblast-DKK3+ sublining (SC-F3) | ENSG00000105664 | COMP | 0.81905082 | 0.71929825 | 0.10470904 |
| SC-F3 | Fibroblast-DKK3+ sublining (SC-F3) | ENSG00000164694 | FNDC1 | 0.8072182 | 0.76315789 | 0.16960184 |
| SC-F3 | Fibroblast-DKK3+ sublining (SC-F3) | ENSG00000131386 | GALNT15 | 0.81963055 | 0.76754386 | 0.15792496 |
| SC-F3 | Fibroblast-DKK3+ sublining (SC-F3) | ENSG00000164294 | GPX8 | 0.80951068 | 0.8245614 | 0.22396631 |
| SC-F3 | Fibroblast-DKK3+ sublining (SC-F3) | ENSG00000167779 | IGFBP6 | 0.83772643 | 0.78947368 | 0.16807044 |
| SC-F3 | Fibroblast-DKK3+ sublining (SC-F3) | ENSG00000130508 | PXDN | 0.83692715 | 0.88157895 | 0.2270291 |
| SC-F3 | Fibroblast-DKK3+ sublining (SC-F3) | ENSG00000168079 | SCARA5 | 0.81990132 | 0.89035088 | 0.3093415 |
| SC-F3 | Fibroblast-DKK3+ sublining (SC-F3) | ENSG00000137573 | SULF1 | 0.83014503 | 0.86403509 | 0.24732006 |
| SC-F3 | Fibroblast-DKK3+ sublining (SC-F3) | ENSG00000091656 | ZFHX4 | 0.78976712 | 0.80263158 | 0.22817764 |

TABLE 6
Differential Gene Expression (Baseline versus Flare) of AC3 Genes Across Four RA Patients

| ensembl | name | name_source | description | p1.logFC | p1.FDR |
|---|---|---|---|---|---|
| ENSG00000002746 | HECW1 | HGNC Symbol | “HECT, C2 and WW | −0.324338654 | 0.572555903 |
| | | | domain containing E3 ubiquitin | | |
| | | | protein ligase 1 | | |
| | | | [Source: HGNC Symbol; | | |
| | | | Acc: HGNC: 22195]” | | |
| ENSG00000004948 | CALCR | HGNC Symbol | calcitonin receptor | −0.383180659 | 0.456241297 |
| | | | [Source: HGNC | | |
| | | | Symbol; Acc: HGNC: 1440] | | |
| ENSG00000006071 | ABCC8 | HGNC Symbol | ATP binding cassette | −0.439216452 | 0.462869018 |
| | | | subfamily C member 8 | | |
| | | | [Source: HGNC Symbol; | | |
| | | | Acc: HGNC: 59] | | |
| ENSG00000006128 | TAC1 | HGNC Symbol | tachykinin precursor | −0.578738859 | 0.363924977 |
| | | | 1 [Source: HGNC | | |
| | | | Symbol; Acc: HGNC: 11517] | | |
| ENSG00000006210 | CX3CL1 | HGNC Symbol | C-X3-C motif chemokine | −0.471842178 | 0.456192862 |
| | | | ligand 1 [Source: | | |
| | | | HGNC Symbol; Acc: | | |
| | | | HGNC: 10647] | | |
| ENSG00000006283 | CACNA1G | HGNC Symbol | calcium voltage-gated | −0.65282151 | 0.174308995 |
| | | | channel subunit alpha1 G | | |
| | | | [Source: HGNC Symbol; | | |
| | | | Acc: HGNC: 1394] | | |
| ENSG00000006788 | MYH13 | HGNC Symbol | myosin heavy chain | −0.516406336 | 0.431394501 |
| | | | 13 [Source: HGNC | | |
| | | | Symbol; Acc: HGNC: 7571] | | |
| ENSG00000009709 | PAX7 | HGNC Symbol | paired box7 [Source: HGNC | −0.287566069 | 0.642117066 |
| | | | Symbol; Acc: HGNC: 8921] | | |
| ENSG00000011677 | GABRA3 | HGNC Symbol | gamma-aminobutyric acid | −0.626857358 | 0.372582084 |
| | | | type A receptor alpha3 | | |
| | | | subunit [Source: HGNC | | |
| | | | Symbol; Acc: HGNC: 4077] | | |
| ENSG00000015520 | NPC1L1 | HGNC Symbol | NPC1 like intracellular cholesterol | −0.449755668 | 0.428701424 |
| | | | transporter 1 | | |
| | | | [Source: HGNC Symbol; | | |
| | | | Acc: HGNC: 7898] | | |
| ENSG00000018607 | ZNF806 | HGNC Symbol | zinc finger protein | −0.534986692 | 0.413023969 |
| | | | 806 [Source: HGNC | | |
| | | | Symbol; Acc: HGNC: 33228] | | |
| ENSG00000018625 | ATP1A2 | HGNC Symbol | ATPase Na+/K+ | −0.380151358 | 0.490411631 |
| | | | transporting subunit alpha 2 | | |
| | | | [Source: HGNC Symbol; | | |
| | | | Acc: HGNC: 800] | | |
| ENSG00000019505 | SYT13 | HGNC Symbol | synaptotagmin 13 [Source: HGNC | −0.846079415 | 0.136236167 |
| | | | Symbol; Acc: HGNC: 14962] | | |
| ENSG00000039139 | DNAH5 | HGNC Symbol | dynein axonemal heavy chain 5 | −0.56614922 | 0.362219403 |
| | | | [Source: HGNC Symbol; | | |
| | | | Acc: HGNC: 2950] | | |
| ENSG00000039987 | BEST2 | HGNC Symbol | bestrophin 2 [Source: HGNC | −0.490981764 | 0.418968005 |
| | | | Symbol; Acc: HGNC: 17107] | | |
| ENSG00000054356 | PTPRN | HGNC Symbol | “protein tyrosine phosphatase, | −0.476009808 | 0.236645395 |
| | | | receptor type N [Source: HGNC | | |
| | | | Symbol; Acc: HGNC: 9676]” | | |
| ENSG00000060656 | PTPRU | HGNC Symbol | “protein tyrosine phasphatase, | −0.440933203 | 0.365704058 |
| | | | receptor type U [Source: HGNC | | |
| | | | Symbol; Acc: HGNC: 9683]” | | |
| ENSG00000060709 | RIMBP2 | HGNC Symbol | RIMS binding protein 2 [Source: HGNC | −0.486388539 | 0.313754361 |
| | | | Symbol; Acc: HGNC: 30339] | | |
| ENSG00000068078 | FGFR3 | HGNC Symbol | fibroblast growth factor receptor 3 | −0.474403299 | 0.318084941 |
| | | | [Source: HGNC Symbol; | | |
| | | | Acc: HGNC: 3690] | | |
| ENSG00000069431 | ABCC9 | HGNC Symbol | ATP binding cassette subfamily C member 9 | −0.755222404 | 0.203283416 |
| | | | [Source: HGNC Symbol; | | |
| | | | Acc: HGNC: 60] | | |
| ENSG00000070886 | EPHA8 | HGNC Symbol | EPH receptor A8 [Source: HGNC | −0.506558126 | 0.338093351 |
| | | | Symbol; Acc: HGNC: 3391] | | |
| ENSG00000072041 | SLC6A15 | HGNC Symbol | solute carrier family 6 member 15 | −0.23655442 | 0.738227188 |
| | | | [Source: HGNC Symbol; | | |
| | | | Acc: HGNC: 13621] | | |
| ENSG00000075891 | PAX2 | HGNC Symbol | paired box 2 [Source: HGNC | −0.768916818 | 0.140011294 |
| | | | Symbol; Acc: HGNC: 8616] | | |
| ENSG00000077080 | ACTL6B | HGNC Symbol | actin like 6B [Source: HGNC | −0.59184773 | 0.349654025 |
| | | | Symbol; Acc: HGNC: 160] | | |
| ENSG00000077522 | ACTN2 | HGNC Symbol | actinin alpha 2 [Source: HGNC | −0.423228174 | 0.460775497 |
| | | | Symbol; Acc: HGNC: 164] | | |
| ENSG00000078295 | ADCY2 | HGNC Symbol | adenylate cyclase 2 [Source: HGNC | −0.360850534 | 0.538385903 |
| | | | Symbol; Acc: HGNC: 233] | | |
| ENSG00000078549 | ADCYAP1R1 | HGNC Symbol | ADCYAP receptor type I [Source: HGNC | −0.265394484 | 0.711597682 |
| | | | Symbol; Acc: HGNC: 242] | | |
| ENSG00000078898 | BPIFB2 | HGNC Symbol | BPI fold containing family B member 2 | −0.585629077 | 0.353356904 |
| | | | [Source: HGNC Symbol; | | |
| | | | Acc: HGNC: 16177] | | |
| ENSG00000079112 | CDH17 | HGNC Symbol | cadherin 17 [Source: HGNC | −0.61532191 | 0.280203448 |
| | | | Symbol; Acc: HGNC: 1756] | | |
| ENSG00000079841 | RIMS1 | HGNC Symbol | regulating synaptic membrane exocytosis 1 | −0.387854778 | 0.537171359 |
| | | | [Source: HGNC Symbol; | | |
| | | | Acc: HGNC: 17282] | | |
| ENSG00000081248 | CACNA1S | HGNC Symbol | calcium voltage-gated channel subunit alpha1 S | −0.562402388 | 0.274520413 |
| | | | [Source: HGNC Symbol; | | |
| | | | Acc: HGNC: 1397] | | |
| ENSG00000081800 | SLC13A1 | HGNC Symbol | solute carrier family 13 member 1 | −0.711148359 | 0.288065754 |
| | | | [Source: HGNC Symbol; | | |
| | | | Acc: HGNC: 10916] | | |
| ENSG00000082175 | PGR | HGNC Symbol | progesterone receptor | −0.458317998 | 0.411160075 |
| | | | [Source: HGNC | | |
| | | | Symbol; Acc: HGNC: 8910] | | |
| ENSG00000084636 | COL16A1 | HGNC Symbol | collagen type XVI alpha 1 chain | −0.798050487 | 0.059725925 |
| | | | [Source: HGNC Symbol; | | |
| | | | Acc: HGNC: 2193] | | |
| ENSG00000088340 | FER1L4 | HGNC Symbol | “fer-1 like family member 4, pseudogene | −0.61617873 | 0.156310461 |
| | | | [Source: HGNC Symbol; Acc: | | |
| | | | HGNC: 15801]” | | |
| ENSG00000089116 | LHX5 | HGNC Symbol | LIM homeobox 5 [Source: HGNC | −0.803770937 | 0.082896409 |
| | | | Symbol; Acc: HGNC: 14216] | | |
| ENSG00000089199 | CHGB | HGNC Symbol | chromogranin B [Source: HGNC | −0.637146342 | 0.263673795 |
| | | | Symbol; Acc: HGNC: 1930] | | |
| ENSG00000089225 | TBX5 | HGNC Symbol | T-box 5 [Source: HGNC Symbol; Acc: | −0.56322196 | 0.348010747 |
| | | | HGNC: 11604] | | |
| ENSG00000091128 | LAMB4 | HGNC Symbol | laminin subunit beta | −0.488177715 | 0.356139517 |
| | | | 4 [Source: HGNC | | |
| | | | Symbol; Acc: HGNC: 6491] | | |
| ENSG00000091137 | SLC2GA4 | HGNC Symbol | solute carrier family 26 member 4 | −0.554256056 | 0.194213389 |
| | | | [Source: HGNC Symbol; | | |
| | | | Acc: HGNC: 8818] | | |
| ENSG00000091656 | ZFHX4 | HGNC Symbol | zinc finger homeobox | −0.447229074 | 0.37818144 |
| | | | 4 [Source: HGNC | | |
| | | | Symbol; Acc: HGNC: 30939] | | |
| ENSG00000095587 | TLL2 | HGNC Symbol | tolloid like 2 [Source: HGNC | −0.409359463 | 0.40882038 |
| | | | Symbol; Acc: HGNC: 11844] | | |
| ENSG00000095637 | SORBS1 | HGNC Symbol | sorbin and SH3 domain containing 1 | −0.324241573 | 0.084190364 |
| | | | [Source: HGNC Symbol; | | |
| | | | Acc: HGNC: 14565] | | |
| ENSG00000099625 | CBARP | HGNC Symbol | CACN beta subunit associated regulatory protein | −0.611658751 | 0.305918323 |
| | | | [Source: HGNC Symbol; | | |
| | | | Acc: HGNC: 28617] | | |
| ENSG00000100033 | PRODH | HGNC Symbol | proline dehydrogenase | −0.729963766 | 0.114187223 |
| | | | 1 [Source: HGNC | | |
| | | | Symbol; Acc: HGNC: 9453] | | |
| ENSG00000100065 | CARD10 | HGNC Symbol | caspase recruitment domain family member 10 | −0.704322375 | 0.116976682 |
| | | | [Source: HGNC Symbol; | | |
| | | | Acc: HGNC: 16422] | | |
| ENSG00000100078 | PLA2G3 | HGNC Symbol | phospholipase A2 group | −0.153902704 | 0.855150771 |
| | | | III [Source: HGNC | | |
| | | | Symbol; Acc: HGNC: 17934] | | |
| ENSG00000100312 | ACR | HGNC Symbol | acrasin [Source: HGNC Symbol; | −0.595590525 | 0.36766088 |
| | | | Acc: HGNC: 126] | | |
| ENSG00000101004 | NINL | HGNC Symbol | ninein like [Source: HGNC | −0.461697247 | 0.040537083 |
| | | | Symbol; Acc: HGNC: 29163] | | |
| ENSG00000101057 | MYBL2 | HGNC Symbol | MYB proto-oncogene like | −0.348283275 | 0.153757922 |
| | | | 2 [Source: HGNC | | |
| | | | Symbol; Acc: HGNC: 7548] | | |
| ENSG00000101203 | COL2OA1 | HGNC Symbol | callagen type XX alpha 1 | −0.611027042 | 0.16213506 |
| | | | chain [Source: HGNC | | |
| | | | Symbol; Acc: HGNC: 14670] | | |
| ENSG00000101276 | SLC52A3 | HGNC Symbol | solute carrier family 52 member 3 | −0.619177881 | 0.319817117 |
| | | | [Source: HGNC Symbol; | | |
| | | | Acc: HGNC: 16187] | | |
| ENSG00000101680 | LAMA1 | HGNC Symbol | laminin subunit alpha | −0.361627137 | 0.534718981 |
| | | | 1 [Source: HGNC | | |
| | | | Symbol; Acc: HGNC: 6481] | | |
| ENSG00000101871 | MID1 | HGNC Symbol | midline 1 [Source: HGNC Symbol; | −0.530388962 | 0.160205439 |
| | | | Acc: HGNC: 7095] | | |
| ENSG00000102287 | GABRE | HGNC Symbol | gamma-aminobutyric acid type A receptor | −0.678792592 | 0.062885659 |
| | | | epsilon subunit [Source: HGNC | | |
| | | | Symbol; Acc: HGNC: 4085] | | |
| ENSG00000102290 | PCDH11X | HGNC Symbol | protocadherin 11 X-linked | −0.646927871 | 0.279252696 |
| | | | [Source: HGNC | | |
| | | | Symbol; Acc: HGNC: 8656] | | |
| ENSG00000102452 | NALCN | HGNC Symbol | “sodiom leak channel, non-selective | −0.587260069 | 0.349930169 |
| | | | [Source: HGNC Symbol; | | |
| | | | Acc: HGNC: 19082]” | | |
| ENSG00000103647 | CORO2B | HGNC Symbol | coronin 2B [Source: HGNC | −0.364156888 | 0.361299257 |
| | | | Symbol; Acc: HGNC: 2256] | | |
| ENSG00000103855 | CD276 | HGNC Symbol | CD276 molecule [Source: HGNC | −0.786492202 | 0.123693703 |
| | | | Symbol; Acc: HGNC: 19137] | | |
| ENSG00000104055 | TGM5 | HGNC Symbol | transglutaminase | −0.744478896 | 0.156122955 |
| | | | 5 [Source: HGNC | | |
| | | | Symbol; Acc: HGNC: 11781] | | |
| ENSG00000104313 | EYA1 | HGNC Symbol | EYA transcriptional coactivator | −0.54795524 | 0.332328891 |
| | | | and phosphatase 1 [Source: | | |
| | | | HGNC Symbol; Acc: HGNC: 3519] | | |
| ENSG00000104435 | STMN2 | HGNC Symbol | stathmin 2 [Source: HGNC | −0.538149899 | 0.377735541 |
| | | | Symbol; Acc: HGNC: 10577] | | |
| ENSG00000104537 | ANXA13 | HGNC Symbol | annexin A13 [Source: HGNC | −0.298697524 | 0.666104371 |
| | | | Symbol; Acc: HGNC: 536] | | |
| ENSG00000104967 | NOVA2 | HGNC Symbol | NOVA alternative splicing regulator 2 | −0.906429895 | 0.09600687 |
| | | | [Source: HGNC Symbol; | | |
| | | | Acc: HGNC: 7887] | | |
| ENSG00000105290 | APLP1 | HGNC Symbol | amyloid beta precursor like protein 1 | −0.626897604 | 0.28126695 |
| | | | [Source: HGNC Symbol; | | |
| | | | Acc: HGNC: 597] | | |
| ENSG00000105357 | MYH14 | HGNC Symbol | myosin heavy chain | −0.396112836 | 0.489919147 |
| | | | 14 [Source: HGNC | | |
| | | | Symbol; Acc: HGNC: 23212] | | |
| ENSG00000105392 | CRX | HGNC Symbol | cone-rod homeobox | −0.652077271 | 0.295697 |
| | | | [Source: HGNC | | |
| | | | Symbol; Acc: HGNC: 2383] | | |
| ENSG00000105549 | THEG | HGNC Symbol | theg spermatid protein | −0.567300418 | 0.345650275 |
| | | | [Source: HGNC | | |
| | | | Symbol; Acc: HGNC: 13706] | | |
| ENSG00000105605 | CACNG7 | HGNC Symbol | calcium voltage-gated channel auxiliary subunit | −0.825394915 | 0.173466676 |
| | | | gamma 7 [Source: | | |
| | | | HGNC Symbol; | | |
| | | | Acc: HGNC: 13626] | | |
| ENSG00000105664 | COMP | HGNC Symbol | cartilage oligomeric | −0.799132187 | 0.027433298 |
| | | | matrix protein | | |
| | | | [Source: HGNC Symbol; | | |
| | | | Acc: HGNC: 2227] | | |
| ENSG00000106078 | COBL | HGNC Symbol | cordon-bleu WH2 rapeat | −0.492882526 | 0.263675081 |
| | | | protein [Source: HGNC | | |
| | | | Symbol; Acc: HGNC: 22199] | | |
| ENSG00000106304 | SPAM1 | HGNC Symbol | sperm adhesion molecule | −0.294698557 | 0.697359656 |
| | | | 1 [Source: HGNC | | |
| | | | Symbol; Acc: HGNC: 11217] | | |
| ENSG00000106648 | GALNTL5 | HGNC Symbol | polypeptide N-acetylgalactosaminyltransferase | −0.610689222 | 0.36620383 |
| | | | like 5 [Source: HGNC Symbol; | | |
| | | | Acc: HGNC: 21725] | | |
| ENSG00000106689 | LHX2 | HGNC Symbol | LIM homeobox 2 [Source: HGNC | −0.630789543 | 0.278431947 |
| | | | Symbol; Acc: HGNC: 6594] | | |
| ENSG00000107295 | SH3GL2 | HGNC Symbol | “SH3 domain containing GRB2 like 2, endophilin | −0.633805457 | 0.350665788 |
| | | | A1 [Source: HGNC Symbol; | | |
| | | | Acc: HGNC: 10831]” | | |
| ENSG00000107807 | TLX1 | HGNC Symbol | T cell leukemia homeobox | −0.773774408 | 0.156228514 |
| | | | 1 [Source: HGNC | | |
| | | | Symbol; Acc: HGNC: 5056] | | |
| ENSG00000108018 | SORCS1 | HGNC Symbol | sortilin relatad VPS10 domain containing | −0.57187073 | 0.324225606 |
| | | | recaptor 1 [Source: HGNC | | |
| | | | Symbol; Acc: HGNC: 16697] | | |
| ENSG00000109101 | FDXN1 | HGNC Symbol | forkhead box N1 [Source: HGNC | −0.551457548 | 0.309062169 |
| | | | Symbol; Acc: HGNC: 12765] | | |
| ENSG00000110786 | PTPN5 | HGNC Symbol | “protein tyrosine phosphatase, non-receptor type | −0.733454811 | 0.20453153 |
| | | | 5 [Source: HGNC | | |
| | | | Symbol; Acc: HGNC: 9657]” | | |
| ENSG00000110887 | DAO | HGNC Symbol | D-amino acid oxidase | −0.519549208 | 0.394229719 |
| | | | [Source: HGNC | | |
| | | | Symbol; Acc: HGNC: 2671] | | |
| ENSG00000110975 | SYT10 | HGNC Symbol | synaptotagmin 10 | −0.84217722 | 0.209970599 |
| | | | [Source: HGNC | | |
| | | | Symbol; Acc: HGNC: 19266] | | |
| ENSG00000111262 | KCNA1 | HGNC Symbol | potassium voltage-gated channel subfamily A | −0.777520439 | 0.155026428 |
| | | | member 1 [Source: HGNC Symbol; | | |
| | | | Acc: HGNC: 6218] | | |
| ENSG00000111799 | COL12A1 | HGNC Symbol | collagen type XII alpha 1 | −0.950476274 | 0.050268873 |
| | | | chain [Source: HGNC | | |
| | | | Symbol; Acc: HGNC: 2188] | | |
| ENSG00000112186 | CAP2 | HGNC Symbol | cyclase associated actin cytoskeleton | −0.981736845 | 0.075743598 |
| | | | regulatory protein | | |
| | | | 2 [Source: HGNC | | |
| | | | Symbol; Acc: HGNC: 20039] | | |
| ENSG00000112238 | PRDM13 | HGNC Symbol | PR/SET domain 13 [Source: HGNC | −0.71815787 | 0.166013748 |
| | | | Symbol; Acc: HGNC: 13998] | | |
| ENSG00000112337 | SLC17A2 | HGNC Symbol | solute carrier family 17 member 2 | −0.701065633 | 0.275381582 |
| | | | [Source: HGNC Symbol; | | |
| | | | Acc: HGNC: 10930] | | |
| ENSG00000112499 | SLC22A2 | HGNC Symbol | solute carrier family 22 member 2 | −0.585273619 | 0.283511169 |
| | | | [Source: HGNC Symbol; | | |
| | | | Acc: HGNC: 10966] | | |
| ENSG00000114019 | AMOTL2 | HGNC Symbol | angiomotin like 2 [Source: HGNC | −0.638655464 | 0.208042203 |
| | | | Symbol; Acc: HGNC: 17812] | | |
| ENSG00000115041 | KCNIP3 | HGNC Symbol | potassium voltage-gated channel interacting | −0.390671123 | 0.447854968 |
| | | | protein 3 [Source: HGNC Symbol; | | |
| | | | Acc: HGNC: 15523] | | |
| ENSG00000115155 | DTOF | HGNC Symbol | otoferlin [Source: HGNC Symbol; | −0.549744866 | 0.121108939 |
| | | | Acc: HGNC: 8515] | | |
| ENSG00000115648 | MLPH | HGNC Symbol | melanophilin [Source: HGNC | −0.344672976 | 0.607643704 |
| | | | Symbol; Acc: HGNC: 29643] | | |
| ENSG00000116176 | TPSG1 | HGNC Symbol | tryptase gamma 1 [Source: HGNC | −0.696648839 | 0.249497404 |
| | | | Symbol; Acc: HGNC: 14134] | | |
| ENSG00000116721 | PRAMEF1 | HGNC Symbol | PRAME family member | −0.909385699 | 0.148992939 |
| | | | 1 [Source: HGNC | | |
| | | | Symbol; Acc: HGNC: 28840] | | |
| ENSG00000116748 | AMPD1 | HGNC Symbol | adenosine monophosphate deaminase 1 | −0.630797468 | 0.331507314 |
| | | | [Source: HGNC Symbol; | | |
| | | | Acc: HGNC: 468] | | |
| ENSG00000116833 | NR5A2 | HGNC Symbol | nuclear receptor subfamily | −0.796732603 | 0.066795635 |
| | | | 5 group A member 2 | | |
| | | | [Source: HGNC Symbol; | | |
| | | | Acc: HGNC: 7984] | | |
| ENSG00000117114 | ADGRL2 | HGNC Symbol | adhesion G protein-coupled | −0.80941013 | 0.08058928 |
| | | | receptor L2 | | |
| | | | [Source: HGNC Symbol; | | |
| | | | Acc: HGNC: 18582] | | |
| ENSG00000117501 | MRDH9 | HGNC Symbol | maestro heat like repeat | −0.770913244 | 0.189731062 |
| | | | family member 9 | | |
| | | | [Source: HGNC Symbol; | | |
| | | | Acc: HGNC: 26287] | | |
| ENSG00000118194 | TNNT2 | HGNC Symbol | “troponin T2, cardiac | −0.546314355 | 0.341682105 |
| | | | type [Source: HGNC | | |
| | | | Symbol; Acc: HGNC: 11949]” | | |
| ENSG00000118733 | DLFM3 | HGNC Symbol | olfactomedin 3 [Source: HGNC | −0.801008321 | 0.203283416 |
| | | | Symbol; Acc: HGNC: 17990] | | |
| ENSG00000119125 | GDA | HGNC Symbol | guanine deaminase [Source: HGNC | −0.747333668 | 0.215289278 |
| | | | Symbol; Acc: HGNC: 4212] | | |
| ENSG00000119283 | TRIM67 | HGNC Symbol | tripartite motif containing | −0.692548587 | 0.118978734 |
| | | | 67 [Source: HGNC | | |
| | | | Symbol; Acc: HGNC: 31859] | | |
| ENSG00000119547 | ONECUT2 | HGNC Symbol | one cut homeobox 2 [Source: HGNC | −0.364064899 | 0.566813453 |
| | | | Symbol; Acc: HGNC: 8139] | | |
| ENSG00000120251 | GRIA2 | HGNC Symbol | glutamate ionotropic receptor | −0.536759703 | 0.393639567 |
| | | | AMPA type subunit 2 [Scorce: | | |
| | | | HGNC Symbol; Acc: HGNC: 4572] | | |
| ENSG00000120332 | TNN | HGNC Symbol | tenascin N [Source: HGNC | −0.797308943 | 0.154902439 |
| | | | Symbol; Acc: HGNC: 22942] | | |
| ENSG00000120738 | EGR1 | HGNC Symbol | Early growth response | 0.387004274 | 0.467739752 |
| | | | 1 [Source: HGNC | | |
| | | | Symbol; Acc: HGNC: 3238] | | |
| ENSG00000121207 | LRAT | HGNC Symbol | lecithin retino acyltransferase | −0.478671249 | 0.448666849 |
| | | | [Source: HGNC | | |
| | | | Symbol; Acc: HGNC: 6685] | | |
| ENSG00000122121 | XPNPEP2 | HGNC Symbol | X-prolyl aminopeptidase | −0.787898058 | 0.044947173 |
| | | | 2 [Source: HGNC | | |
| | | | Symbol; Acc: HGNC: 12823] | | |
| ENSG00000122778 | KIAA1549 | HGNC Symbol | KIAA1549 [Source: HGNC Symbol; | −0.516890518 | 0.188627673 |
| | | | Acc: HGNC: 22219] | | |
| ENSG00000123243 | ITIH5 | HGNC Symbol | inter-alpha-trypsin inhibitor | −0.571168831 | 0.310806911 |
| | | | heavy chain family | | |
| | | | member 5 [Source: HGNC | | |
| | | | Symbol; Acc: HGNC: 21449] | | |
| ENSG00000123572 | NRK | HGNC Symbol | Nik related kinase | −0.486349518 | 0.420910239 |
| | | | [Source: HGNC | | |
| | | | Symbol; Acc: HGNC: 25391] | | |
| ENSG00000123977 | DAW1 | HGNC Symbol | dyein assembly factor | −0.654419876 | 0.276698737 |
| | | | with WD repeats 1 | | |
| | | | [Source: HGNC Sym8l; | | |
| | | | Acc: HGNC: 26383] | | |
| ENSG00000124092 | CTCFL | HGNC Symbol | CCCTC-binding factor | −0.443308442 | 0.467030393 |
| | | | like [Source: HGNC | | |
| | | | Symbol; Acc: HGNC: 16234] | | |
| ENSG00000124253 | PCK1 | HGNC Symbol | phosphoenolpyruvate | −0.720216315 | 0.251094538 |
| | | | carboxykinase 1 | | |
| | | | [Source: HGNC Symbol; | | |
| | | | Acc: HGNC: 8724] | | |
| ENSG00000124440 | HIF3A | HGNC Symbol | hypoxia inducible factor | −0.743934584 | 0.170117086 |
| | | | 3 alpha subunit | | |
| | | | [Source: HGNC Symbol; | | |
| | | | Acc: HGNC: 15825] | | |
| ENSG00000124466 | LYPD3 | HGNC Symbol | LY6/PLAUR domain containing 3 | −0.753602142 | 0.045770113 |
| | | | [Source: HGNC Symbol; | | |
| | | | Acc: HGNC: 24880] | | |
| ENSG00000124749 | COL21A1 | HGNC Symbol | collagen type XXI alpha 1 chain | −0.382109802 | 0.564364144 |
| | | | [Source: HGNC Symbol; | | |
| | | | Acc: HGNC: 17025] | | |
| ENSG00000125255 | SLC10A2 | HGNC Symbol | solute carrier family 10 member 2 | −0.716865675 | 0.262922534 |
| | | | [Source: HGNC Symbol; | | |
| | | | Acc: HGNC: 10906] | | |
| ENSG00000125492 | BARHL1 | HGNC Symbol | BarH like homeobox | −0.153912437 | 0.823410461 |
| | | | 1 [Source: HGNC | | |
| | | | Symbol; Acc: HGNC: 953] | | |
| ENSG00000125740 | FOSB | HGNC Symbol | “FosB proto-oncogene, AP-1 | 0.168490461 | 0.82332641 |
| | | | transcription factor subunit | | |
| | | | [Source: HGNC Symbol; | | |
| | | | Acc: HGNC: 3797]” | | |
| ENSG00000127129 | EDN2 | HGNC Symbol | endothelin 2 [Source: HGNC | −0.396163266 | 0.500431956 |
| | | | Symbol; Acc: HGNC: 3177] | | |
| ENSG00000128573 | FOXP2 | HGNC Symbol | forkhhead box P2 [Source: HGNC | −0.425628065 | 0.496663487 |
| | | | Symbol; Acc: HGNC: 13875] | | |
| ENSG00000128917 | DLL4 | HGNC Symbol | delta like canonical Notch ligand 4 | −0.702456843 | 0.136145207 |
| | | | [Source: HGNC | | |
| | | | Symbol; Acc: HGNC: 2910] | | |
| ENSG00000129946 | SHC2 | HGNC Symbol | SHC adaptor protein 2 [Source: HGNC | −0.843357896 | 0.081141031 |
| | | | Symbol; Acc: HGNC: 29869] | | |
| ENSG00000129990 | SYT5 | HGNC Symbol | synaptotagmin 5 [Source: HGNC | −0.81970687 | 0.165777377 |
| | | | Symbol; Acc: HGNC: 11513] | | |
| ENSG00000130045 | NXNL2 | HGNC Symbol | nucleoredoxin like 2 [Source: HGNC | −0.59403632 | 0.278225713 |
| | | | Symbol; Acc: HGNC: 30482] | | |
| ENSG00000130226 | DPP6 | HGNC Symbol | dipeptidyl peptidase | −0.086732477 | 0.915560052 |
| | | | like 6 [Source: HGNC | | |
| | | | Symbol; Acc: HGNC: 3010] | | |
| ENSG00000130287 | NCAN | HGNC Symbol | neurocan [Source: HGNC Symbol; | −0.665537698 | 0.281766504 |
| | | | Acc: HGNC: 2465] | | |
| ENSG00000130477 | UNC13A | HGNC Symbol | unc-13 homolog A [Source: HGNC | −0.493441944 | 0.309175629 |
| | | | Symbol; Acc: HGNC: 23150] | | |
| ENSG00000130508 | PXDN | HGNC Symbol | paroxidasin [Source: HGNC | −0.703310686 | 0.123498419 |
| | | | Symbol; Acc: HGNC: 14966] | | |
| ENSG00000130528 | HRC | HGNC Symbol | histidine rich calcium | −0.692484137 | 0.295903841 |
| | | | binding protein | | |
| | | | [Source: HGNC Symbol; | | |
| | | | Acc: HGNC: 5178] | | |
| ENSG00000130540 | SULT4A1 | HGNC Symbol | sulfotransferase | −0.672278031 | 0.247255807 |
| | | | family 4A member 1 | | |
| | | | [Source: HGNC Symbol; | | |
| | | | Acc: HGNC: 14903] | | |
| ENSG00000130635 | COL5A1 | HGNC Symbol | collagan type V alpha 1 chain | −1.059536259 | 0.005145777 |
| | | | [Source: HGNC Symbol; | | |
| | | | Acc: HGNC: 2209] | | |
| ENSG00000131044 | TTLL9 | HGNC Symbol | tubulin tyrosine ligase like 9 | −0.648555965 | 0.080577407 |
| | | | [Source: HGNC Symbol; | | |
| | | | Acc: HGNC: 16118] | | |
| ENSG00000131183 | SLD34A1 | HGNC Symbol | solute carrier family 34 member 1 | −0.553608399 | 0.222662052 |
| | | | [Source: HGNC Symbol; | | |
| | | | Acc: HGNC: 11019] | | |
| ENSG00000131386 | GALNT15 | HGNC Symbol | polypeptide | −0.249199422 | 0.57936334 |
| | | | N-acetylgalactosaminyltransferase | | |
| | | | 15 [Source: HGNC Symbol; | | |
| | | | Acc: HGNC: 21531] | | |
| ENSG00000132297 | HHLA1 | HGNC Symbol | HERV-H LTR-associating | −0.569303097 | 0.342618221 |
| | | | 1 [Source: HGNC | | |
| | | | Symbol; Acc: HGNC: 4904] | | |
| ENSG00000132321 | IQCA1 | HGNC Symbol | IQ motif containing | −0.491444835 | 0.302209736 |
| | | | with AAA domain 1 | | |
| | | | [Source: HGNC Symbol; | | |
| | | | Acc: HGNC: 26195] | | |
| ENSG00000132639 | SNAP25 | HGNC Symbol | synaptosome associated protein 25 | −0.484337596 | 0.393579181 |
| | | | [Source: HGNC Symbol; | | |
| | | | Acc: HGNC: 11132] | | |
| ENSG00000132972 | RNF17 | HGNC Symbol | ring finger protein | −0.539876442 | 0.323158258 |
| | | | 17 [Source: HGNC | | |
| | | | Symbol; Acc: HGNC: 10060] | | |
| ENSG00000132975 | GPR12 | HGNC Symbol | G protein-coupled receptor | −0.559671507 | 0.380221581 |
| | | | 12 [Source: HGNC | | |
| | | | Symbol; Acc: HGNC: 4466] | | |
| ENSG00000133083 | DCLK1 | HGNC Symbol | doublecortin like kinase | −0.693023499 | 0.188550204 |
| | | | 1 [Source: HGNC | | |
| | | | Symbol; Acc: HGNC: 2700] | | |
| ENSG00000133110 | POSTN | HGNC Symbol | periostin [Source: HGNC Symbol; | −1.95711401 | 0.00141871 |
| | | | Acc: HGNC: 16953] | | |
| ENSG00000133124 | IRS4 | HGNC Symbol | insulin recaptor substrate | −0.486576337 | 0.415362956 |
| | | | 4 [Source: HGNC | | |
| | | | Symbol; Acc: HGNC: 6128] | | |
| ENSG00000133454 | MYO18B | HGNC Symbol | myosin XVIIIB [Source: HGNC | −0.556047442 | 0.119461856 |
| | | | Symbol; Acc: HGNC: 18150] | | |
| ENSG00000134240 | HMGCS2 | HGNC Symbol | 3-hydroxy-3-mathylglotaryl-CoA | −0.569886518 | 0.322294501 |
| | | | synthase 2 | | |
| | | | [Source: HGNC Symbol; | | |
| | | | Acc: HGNC: 5008] | | |
| ENSG00000134871 | COL4A2 | HGNC Symbol | collagen type IV alpha 2 | −0.723958351 | 0.078843824 |
| | | | chain [Source: HGNC | | |
| | | | Symbol; Acc: HGNC: 2203] | | |
| ENSG00000135097 | MSI1 | HGNC Symbol | musashi RNA binding protein | −0.681488708 | 0.233945897 |
| | | | 1 [Source: HGNC | | |
| | | | Symbol; Acc: HGNC: 7330] | | |
| ENSG00000135409 | AMHR2 | HGNC Symbol | anti-Mullerian hormone | −0.365902217 | 0.57936334 |
| | | | receptor type 2 | | |
| | | | [Source: HGNC Symbol; | | |
| | | | Acc: HGNC: 465] | | |
| ENSG00000135454 | B4GALNT1 | HGNC Symbol | “beta-1,4-N-acetyl- | −0.838916247 | 0.094761195 |
| | | | galactosaminyltransferase 1 | | |
| | | | [Source: HGNC Symbol; | | |
| | | | Acc: HGNC: 4117]” | | |
| ENSG00000135472 | FAIM2 | HGNC Symbol | Fas apoptotic inhibitory molecule 2 | −0.683241772 | 0.166816589 |
| | | | [Source: HGNC Symbol; | | |
| | | | Acc: HGNC: 17067] | | |
| ENSG00000135917 | SLC19A3 | HGNC Symbol | solute carrier family 19 member 3 | −0.940946251 | 0.097071433 |
| | | | [Source: HGNC Symbol; | | |
| | | | Acc: HGNC: 16266] | | |
| ENSG00000135931 | ARMC9 | HGNC Symbol | armadillo repeat containing | −0.671965301 | 0.040773449 |
| | | | 9 [Source: HGNC | | |
| | | | Symbol; Acc: HGNC: 20730] | | |
| ENSG00000136352 | NKX2-1 | HGNC Symbol | NK2 homeobox 1 [Source: HGNC | −0.828072024 | 0.143100586 |
| | | | Symbol; Acc: HGNC: 11825] | | |
| ENSG00000136535 | TBR1 | HGNC Symbol | “T-box, brain | −0.569543676 | 0.291480115 |
| | | | 1 [Source: HGNC | | |
| | | | Symbol; Acc: HGNC: 11590]” | | |
| ENSG00000136574 | GATA4 | HGNC Symbol | GATA binding protein | −0.431785431 | 0.458893715 |
| | | | 4 [Source: HGNC | | |
| | | | Symbol; Acc: HGNC: 4173] | | |
| ENSG00000136695 | IL36RN | HGNC Symbol | interleukin 36 receptor antagonist | −0.111115253 | 0.887785479 |
| | | | [Source: HGNC Symbol; | | |
| | | | Acc: HGNC: 15561] | | |
| ENSG00000137203 | TFAP2A | HGNC Symbol | transcription factor AP-2 alpha | −0.719606178 | 0.197926578 |
| | | | [Source: HGNC Symbol; | | |
| | | | Acc: HGNC: 11742] | | |
| ENSG00000137573 | SULF1 | HGNC Symbol | sulfatase 1 [Source: HGNC | −0.721098487 | 0.155312568 |
| | | | Symbol; Acc: HGNC: 20391] | | |
| ENSG00000137648 | TMPRSS4 | HGNC Symbol | transmembrane serine protease 4 | −0.697399848 | 0.100309089 |
| | | | [Source: HGNC Symbol; | | |
| | | | Acc: HGNC: 11878] | | |
| ENSG00000137766 | UNC13C | HGNC Symbol | unc-13 homolog C [Source: HGNC | −0.345910067 | 0.592753582 |
| | | | Symbol; Acc: HGNC: 23149] | | |
| ENSG00000137819 | PAQR5 | HGNC Symbol | progestin and adipoQ receptor | −0.466098604 | 0.254871322 |
| | | | family member 5 | | |
| | | | [Source: HGNC Symbol; | | |
| | | | Acc: HGNC: 29645] | | |
| ENSG00000138162 | TACC2 | HGNC Symbol | transforming acidic | −0.598212058 | 0.215791557 |
| | | | coiled-coil containing | | |
| | | | protein 2 [Source: HGNC Symbol; | | |
| | | | Acc: HGNC: 11523] | | |
| ENSG00000138650 | PCDH10 | HGNC Symbol | protocadherin 10 [Source: HGNC | −0.339300016 | 0.604591273 |
| | | | Symbol; Acc: HGNC: 13404] | | |
| ENSG00000138675 | FGF5 | HGNC Symbol | fibroblast growth factor | −0.605345738 | 0.362679738 |
| | | | 5 [Source: HGNC | | |
| | | | Symbol; Acc: HGNC: 3683] | | |
| ENSG00000138759 | FRAS1 | HGNC Symbol | Fraser extracellular matrix | −0.222946616 | 0.747027813 |
| | | | complex subunit 1 | | |
| | | | [Source: HGNC Symbol; | | |
| | | | Acc: HGNC: 19185] | | |
| ENSG00000138892 | TTLL8 | HGNC Symbol | tubulin tyrosine ligase like | −0.897768588 | 0.092191936 |
| | | | 8 [Source: HGNC | | |
| | | | Symbol; Acc: HGNC: 34000] | | |
| ENSG00000139144 | PIK3C2G | HGNC Symbol | phosphatidylinositol-4-phosphate | −0.156595171 | 0.833030115 |
| | | | 3-kinase catalytic subunit type | | |
| | | | 2 gamma [Source: HGNC | | |
| | | | Symbol; Acc: HGNC: 8973] | | |
| ENSG00000139220 | PPF1A2 | HGNC Symbol | PTPRF interacting protein alpha 2 | −0.378470282 | 0.536515358 |
| | | | [Source: HGNC Symbol; | | |
| | | | Acc: HGNC: 9246] | | |
| ENSG00000139767 | SRRM4 | HGNC Symbol | serine/arginine repetitive matrix 4 | −0.3343885 | 0.60876368 |
| | | | [Source: HGNC Symbol; | | |
| | | | Acc: HGNC: 29389] | | |
| ENSG00000139865 | TTC6 | HGNC Symbol | tetratricopeptide repeat domain 6 | −0.541511875 | 0.377281018 |
| | | | [Source: HGNC Symbol; | | |
| | | | Acc: HGNC: 19739] | | |
| ENSG00000140279 | DUOX2 | HGNC Symbol | dual oxidase 2 [Source: HGNC | −0.606118929 | 0.157597775 |
| | | | Symbol; Acc: HGNC: 13273] | | |
| ENSG00000140527 | WDR93 | HGNC Symbol | WD repeat domain 93 [Source: HGNC | −0.510238996 | 0.36380238 |
| | | | Symbol; Acc: HGNC: 26924] | | |
| ENSG00000140600 | SH3GL3 | HGNC Symbol | “SH3 domain containing | −0.348680711 | 0.57659031 |
| | | | GRB2 like 3, endophilin | | |
| | | | A3 [Source: HGNC Symbol; | | |
| | | | Acc: HGNC: 10832]” | | |
| ENSG00000141434 | MEP1B | HGNC Symbol | meprin A subunit beta [Source: HGNC | −0.835496908 | 0.100858384 |
| | | | Symbol; Acc: HGNC: 7020] | | |
| ENSG00000141668 | CBLN2 | HGNC Symbol | cerebellin 2 precursor [Source: HGNC | −0.408816792 | 0.515136432 |
| | | | Symbol; Acc: HGNC: 1544] | | |
| ENSG00000141946 | ZIM3 | HGNC Symbol | zinc finger imprinted 3 [Source: HGNC | −0.553199901 | 0.418027916 |
| | | | Symbol; Acc: HGNC: 16366] | | |
| ENSG00000142408 | CACNG8 | HGNC Symbol | calcium voltage-gated channel auxiliary | −0.654089337 | 0.014843697 |
| | | | subunit gamma 8 [Source: HGNC | | |
| | | | Symbol; Acc: HGNC: 13628] | | |
| ENSG00000142449 | FBN3 | HGNC Symbol | fibrillin 3 [Source: HGNC Symbol; | −0.447192665 | 0.423778362 |
| | | | Acc: HGNC: 18794] | | |
| ENSG00000142549 | IGLON5 | HGNC Symbol | IgLON family member 5 [Source: HGNC | −0.544479933 | 0.396566456 |
| | | | Symbol; Acc: HGNC: 34550] | | |
| ENSG00000142611 | PRDM16 | HGNC Symbol | PR/SET domain 16 [Source: HGNC | −0.743776119 | 0.110743572 |
| | | | Symbol; Acc: HGNC: 14000] | | |
| ENSG00000142623 | PADI1 | HGNC Symbol | peptidyl arginine deiminase | −0.616861527 | 0.338644986 |
| | | | 1 [Source: HGNC | | |
| | | | Symbol; Acc: HGNC: 18367] | | |
| ENSG00000142675 | CNKSR1 | HGNC Symbol | connector enhancer of kinase | −0.498623814 | 0.138703112 |
| | | | suppressor of Ras 1 [Source: | | |
| | | | HGNC Symbol; Acc: HGNC: 19700] | | |
| ENSG00000142910 | TINAGL1 | HGNC Symbol | tubulointerstitial nephritis | −0.695391991 | 0.158084486 |
| | | | antigen like 1 | | |
| | | | [Source: HGNC Symbol; | | |
| | | | Acc: HGNC: 19168] | | |
| ENSG00000143107 | FNDC7 | HGNC Symbol | fibronectin type III | −0.533451774 | 0.237237415 |
| | | | domain containing 7 | | |
| | | | [Source: HGNC Symbol; | | |
| | | | Acc: HGNC: 26668] | | |
| ENSG00000143355 | LHX9 | HGNC Symbol | LIM homeobox 9 [Source: HGNC | −0.481769357 | 0.365710069 |
| | | | Symbol; Acc: HGNC: 14222] | | |
| ENSG00000143450 | OAZ3 | HGNC Symbol | ornithine decarboxylase antizyme 3 | −0.731131098 | 0.04678294 |
| | | | [Source: HGNC Symbol; | | |
| | | | Acc: HGNC: 8097] | | |
| ENSG00000143469 | SYT14 | HGNC Symbol | synaptotagmin 14 [Source: HGNC | −0.529977675 | 0.429615839 |
| | | | Symbol; Acc: HGNC: 23143] | | |
| ENSG00000143867 | DSR1 | HGNC Symbol | odd-skipped related | −0.350861532 | 0.593032035 |
| | | | transciption factor 1 | | |
| | | | [Source: HGNC Symbol; | | |
| | | | Acc: HGNC: 8111] | | |
| ENSG00000144115 | THNSL2 | HGNC Symbol | threonine synthase like | −0.327316109 | 0.532009313 |
| | | | 2 [Source: HGNC | | |
| | | | Symbol; Acc: HGNC: 25602] | | |
| ENSG00000144488 | ESPNL | HGNC Symbol | espin like [Source: HGNC | −0.577630971 | 0.253952213 |
| | | | Symbol; Acc: HGNC: 27937] | | |
| ENSG00000144648 | ACKR2 | HGNC Symbol | atypical chemokine receptor | −0.477303908 | 0.045840057 |
| | | | 2 [Source: HGNC | | |
| | | | Symbol; Acc: HGNC: 1565] | | |
| ENSG00000144681 | STAC | HGNC Symbol | SH3 and cysteine rich | −0.560050605 | 0.182021883 |
| | | | domain [Source: HGNC | | |
| | | | Symbol; Acc: HGNC: 11353] | | |
| ENSG00000144730 | IL17RD | HGNC Symbol | interleukin 17 receptor | −0.561060798 | 0.294689324 |
| | | | D [Source: HGNC | | |
| | | | Symbol; Acc: HGNC: 17616] | | |
| ENSG00000144820 | ADGRG7 | HGNC Symbol | adhesion G protein- | −0.492313851 | 0.436137212 |
| | | | coupled receptor G7 | | |
| | | | [Source: HGNC Symbol; | | |
| | | | Acc: HGNC: 19241] | | |
| ENSG00000144908 | ALDH1L1 | HGNC Symbol | aldehyde dehydrogenase | −0.412949182 | 0.450510268 |
| | | | 1 family member L1 | | |
| | | | [Source: HGNC Symbol; | | |
| | | | Acc: HGNC: 3978] | | |
| ENSG00000145242 | EPHA5 | HGNC Symbol | EPH receptor A5 [Source: HGNC | −0.56028279 | 0.397803597 |
| | | | Symbol; Acc: HGNC: 3389] | | |
| ENSG00000145526 | CDH18 | HGNC Symbol | cadherin 18 [Source: HGNC | −0.747075064 | 0.255000128 |
| | | | Symbol; Acc: HGNC: 1757] | | |
| ENSG00000145642 | SHISAL2B | HGNC Symbol | shisa like 2B [Source: HGNC | −1.490085497 | 0.001461516 |
| | | | Symbol; Acc: HGNC: 34236] | | |
| ENSG00000145832 | SLC25A48 | HGNC Symbol | solute carrier family 25 member 48 | −0.382607037 | 0.565480846 |
| | | | [Source: HGNC Symbol; | | |
| | | | Acc: HGNC: 30451] | | |
| ENSG00000146005 | PSD2 | HGNC Symbol | pleckstrin and Sec7 | −0.474391279 | 0.322965191 |
| | | | domain containing 2 | | |
| | | | [Source: HGNC Symbol; | | |
| | | | Acc: HGNC: 19092] | | |
| ENSG00000146521 | LINC01558 | HGNC Symbol | long intergenic non-protein | −0.930731184 | 0.104996052 |
| | | | coding RNA 1558 | | |
| | | | [Source: HGNC Symbol; | | |
| | | | Acc: HGNC: 21235] | | |
| ENSG00000146648 | EGFR | HGNC Symbol | epidermal growth factor receptor | −0.464347302 | 0.323083336 |
| | | | [Source: HGNC Symbol; | | |
| | | | Acc: HGNC: 3236] | | |
| ENSG00000146839 | ZAN | HGNC Symbol | zonadhesin (gene/pseudogene) | −0.501901018 | 0.397563388 |
| | | | [Source: HGNC | | |
| | | | Symbol; Acc: HGNC: 12857] | | |
| ENSG00000146950 | SHROOM2 | HGNC Symbol | shroom family member | −0.547481224 | 0.153040816 |
| | | | 2 [Source: HGNC | | |
| | | | Symbol; Acc: HGNC: 630] | | |
| ENSG00000147573 | TRIM55 | HGNC Symbol | tripartite motif containing | −0.406221036 | 0.529220394 |
| | | | 55 [Source: HGNC | | |
| | | | Symbol; Acc: HGNC: 14215] | | |
| ENSG00000147655 | RSPD2 | HGNC Symbol | R-spondin 2 [Source: HGNC | −0.425816624 | 0.490916964 |
| | | | Symbol; Acc: HGNC: 28583] | | |
| ENSG00000147689 | FAM83A | HGNC Symbol | family with sequence similarity | −0.471846383 | 0.295535482 |
| | | | 83 member A [Source: HGNC | | |
| | | | Symbol; Acc: HGNC: 28210] | | |
| ENSG00000148357 | HMCN2 | HGNC Symbol | hemicentin 2 [Source: HGNC | −0.535566286 | 0.290997361 |
| | | | Symbol; Acc: HGNC: 21293] | | |
| ENSG00000148408 | CACNA1B | HGNC Symbol | calcium voltage-gated channel | −0.501466836 | 0.284867302 |
| | | | subunit alpha1 B [Source: HGNC | | |
| | | | Symbol; Acc: HGNC: 1389] | | |
| ENSG00000148513 | ANKRD30A | HGNC Symbol | ankyrin repeat domain | −0.871448013 | 0.164735964 |
| | | | 30A [Source: HGNC | | |
| | | | Symbol; Acc: HGNC: 17234] | | |
| ENSG00000148702 | HABP2 | HGNC Symbol | hyaluronan binding protien | −0.563571725 | 0.372582084 |
| | | | 2 [Source: HGNC | | |
| | | | Symbol; Acc: HGNC: 4798] | | |
| ENSG00000148342 | SLC5A12 | HGNC Symbol | solute carrier family 5 member 12 | −0.603891229 | 0.317483763 |
| | | | [Source: HGNC Symbol; | | |
| | | | Acc: HGNC: 28750] | | |
| ENSG00000149633 | KIAA1755 | HGNC Symbol | KIAA1755 [Source: HGNC | −0.626269466 | 0.295535482 |
| | | | Symbol; Acc: HGNC: 29372] | | |
| ENSG00000149654 | CDH22 | HGNC Symbol | cadherin 22 [Source: HGNC | −0.38181549 | 0.564754244 |
| | | | Symbol; Acc: HGNC: 13251] | | |
| ENSG00000149926 | FAM57B | HGNC Symbol | family with sequence similarity | −0.826512075 | 0.147540268 |
| | | | 57 member B [Source: HGNC | | |
| | | | Symbol; Acc: HGNC: 25295] | | |
| ENSG00000150086 | NA | NA | NA | −0.381789129 | 0.543943649 |
| ENSG00000150893 | FREM2 | HGNC Symbol | FRAS1 related extracellular | −0.27918432 | 0.685530175 |
| | | | matrix protein 2 [Source: | | |
| | | | HGNC Symbol; Acc: HGNC: 25396] | | |
| ENSG00000151224 | MAT1A | HGNC Symbol | methionine adenosyltransferase 1A | −0.695829857 | 0.25715468 |
| | | | [Source: HGNC Symbol; | | |
| | | | Acc: HGNC: 6903] | | |
| ENSG00000151474 | FRMD4A | HGNC Symbol | FERM domain containing | −0.266779579 | 0.183727706 |
| | | | 4A [Source: HGNC | | |
| | | | Symbol; Acc: HGNC: 25491] | | |
| ENSG00000151572 | ANO4 | HGNC Symbol | anoctamin 4 [Source: HGNC | −0.427488192 | 0.474520384 |
| | | | Symbol; Acc: HGNC: 23837] | | |
| ENSG00000152822 | GRM1 | HGNC Symbol | glutamate metabotropic receptor 1 | −0.560201862 | 0.321661526 |
| | | | [Source: HGNC Symbol; | | |
| | | | Acc: HGNC: 4593] | | |
| ENSG00000152910 | CNTNAP4 | HGNC Symbol | contactin associated protein like 4 | −0.144213925 | 0.859543676 |
| | | | [Source: HGNC Symbol; | | |
| | | | Acc: HGNC: 18747] | | |
| ENSG00000153165 | RGPD3 | HGNC Symbol | RANBP2-like and GRIP | −0.866436535 | 0.003638752 |
| | | | domain containing 3 | | |
| | | | [Source: HGNC Symbol; | | |
| | | | Acc: HGNC: 32416] | | |
| ENSG00000154099 | DNAAF1 | HGNC Symbol | dynein axonemal assembly factor 1 | −0.616204423 | 0.217696484 |
| | | | [Source: HGNC Symbol; | | |
| | | | Acc: HGNC: 30539] | | |
| ENSG00000154478 | GPR2G | HGNC Symbol | G protein-coupled receptor | −0.494806205 | 0.406590218 |
| | | | 26 [Source: HGNC | | |
| | | | Symbol; Acc: HGNC: 4481] | | |
| ENSG00000155816 | FMN2 | HGNC Symbol | formin 2 [Source: HGNC Symbol; | −0.411373015 | 0.528626826 |
| | | | Acc: HGNC: 14074] | | |
| ENSG00000156076 | WIF1 | HGNC Symbol | WNT inhibitory factor 1 | −0.701995845 | 0.249971053 |
| | | | [Source: HGNC | | |
| | | | Symbol; Acc: HGNC: 18081] | | |
| ENSG00000156103 | MMP16 | HGNC Symbol | matrix metallopeptidase | −0.612940919 | 0.354953317 |
| | | | 16 [Source: HGNC | | |
| | | | Symbol; Acc: HGNC: 7162] | | |
| ENSG00000156222 | SLC28A1 | HGNC Symbol | solute carrier family 28 member 1 | −0.484780444 | 0.394304044 |
| | | | [Source: HGNC Symbol; | | |
| | | | Acc: HGNC: 11001] | | |
| ENSG00000156413 | FUT6 | HGNC Symbol | fucosyltransferase | −0.59042874 | 0.291768914 |
| | | | 6 [Source: HGNC | | |
| | | | Symbol; Acc: HGNC: 4017] | | |
| ENSG00000156687 | UNC5D | HGNC Symbol | unc-5 netrin receptor | −0.678558516 | 0.219237459 |
| | | | D [Source: HGNC | | |
| | | | Symbol; Acc: HGNC: 18634] | | |
| ENSG00000157214 | STEAP2 | HGNC Symbol | STEAP2 metalloreductase | −0.751247183 | 0.154094301 |
| | | | [Source: HGNC | | |
| | | | Symbol; Acc: HGNC: 17885] | | |
| ENSG00000157423 | HYDIN | HGNC Symbol | “HYDIN, axonemal central pair | −0.339331962 | 0.501656362 |
| | | | apparatus protein | | |
| | | | [Source: HGNC Symbol; | | |
| | | | Acc: HGNC: 19368]” | | |
| ENSG00000157927 | RADIL | HGNC Symbol | Rap associating with DIL domain | −0.542930642 | 0.218274573 |
| | | | [Source: HGNC Symbol; | | |
| | | | Acc: HGNC: 22226] | | |
| ENSG00000158077 | NLRP14 | HGNC Symbol | NLR family pyrin | −0.399560124 | 0.501906622 |
| | | | domain containing 14 | | |
| | | | [Source: HGNC Symbol; | | |
| | | | Acc: HGNC: 22939] | | |
| ENSG00000158125 | XDH | HGNC Symbol | xanthine dehydrogenase | −0.856880361 | 0.134914762 |
| | | | [Source: HGNC | | |
| | | | Symbol; Acc: HGNC: 12805] | | |
| ENSG00000158258 | CLSTN2 | HGNC Symbol | calsyntenin 2 [Source: HGNC | −0.472246595 | 0.416827728 |
| | | | Symbol; Acc: HGNC: 17448] | | |
| ENSG00000159251 | ACTC1 | HGNC Symbol | “actin, alpha, | −0.407779393 | 0.500035053 |
| | | | cardiac muscle 1 | | |
| | | | [Source: HGNC Symbol; | | |
| | | | Acc: HGNC: 143]” | | |
| ENSG00000159337 | PLA2G4D | HGNC Symbol | phospholipase A2 group | −0.623844111 | 0.222267492 |
| | | | IVD [Source: HGNC | | |
| | | | Symbol; Acc: HGNC: 30038] | | |
| ENSG00000159650 | UROC1 | HGNC Symbol | urocanate hydratase | −0.592610834 | 0.295239234 |
| | | | 1 [Source: HGNC | | |
| | | | Symbol; Acc: HGNC: 26444] | | |
| ENSG00000160460 | SPTBN4 | HGNC Symbol | “spectrin beta, | −0.681223524 | 0.093882704 |
| | | | non-erythrocytic 4 | | |
| | | | [Source: HGNC Symbol; | | |
| | | | Acc: HGNC: 14896]” | | |
| ENSG00000160994 | CCDC105 | HGNC Symbol | coiled-coil domain containing 105 | −0.455466884 | 0.442646097 |
| | | | [Source: HGNC Symbol; | | |
| | | | Acc: HGNC: 26866] | | |
| ENSG00000161103 | AC008132.1 | Clone-based | | −0.52329433 | 0.317152409 |
| | | (Ensembl) gene | | | |
| ENSG00000161243 | FBXO27 | HGNC Symbol | F-box protein 27 [Source: HGNC | −0.817434469 | 0.165930422 |
| | | | Symbol; Acc: HGNC: 18753] | | |
| ENSG00000161270 | NPHS1 | HGNC Symbol | “NPHS1, nephrin [Source: HGNC | −0.59448236 | 0.304121161 |
| | | | Symbol; Acc: HGNC: 7908]” | | |
| ENSG00000161609 | CCDC155 | HGNC Symbol | coiled-coil domain containing 155 | −0.559684333 | 0.313465888 |
| | | | [Source: HGNC Symbol; | | |
| | | | Acc: HGNC: 26520] | | |
| ENSG00000161682 | FAM171A2 | HGNC Symbol | family with sequence | −0.837068643 | 0.064801456 |
| | | | similarity 171 member A2 | | |
| | | | [Source: HGNC Symbol; | | |
| | | | Acc: HGNC: 30480] | | |
| ENSG00000161940 | BCL6B | HGNC Symbol | B cell CLL/lymphoma | −0.733676229 | 0.061835527 |
| | | | 6B [Source: HGNC | | |
| | | | Symbol; Acc: HGNC: 1002] | | |
| ENSG00000162006 | MSLNL | HGNC Symbol | mesothelin-like [Source: HGNC | −0.824168169 | 0.109148457 |
| | | | Symbol; Acc: HGNC: 14170] | | |
| ENSG00000162062 | TEDC2 | HGNC Symbol | tubulin epsilon and delta complex 2 | −0.659328987 | 0.150977424 |
| | | | [Source: HGNC Symbol; | | |
| | | | Acc: HGNC: 25849] | | |
| ENSG00000162510 | MATN1 | HGNC Symbol | “matrilin 1, cartilage | −0.622999455 | 0.135310926 |
| | | | matrix protein | | |
| | | | [Source: HGNC Symbol; | | |
| | | | Acc: HGNC: 6907]” | | |
| ENSG00000162631 | NTNG1 | HGNC Symbol | netrin G1 [Source: HGNC Symbol; | −0.620320047 | 0.187276211 |
| | | | Acc: HGNC: 23319] | | |
| ENSG00000162706 | CADM3 | HGNC Symbol | cell adhesion molecule | −0.682331042 | 0.208106324 |
| | | | 3 [Source: HGNC | | |
| | | | Symbol; Acc: HGNC: 17601] | | |
| ENSG00000162738 | VANGL2 | HGNC Symbol | VANGL planar cell polarity protein 2 | −0.566370045 | 0.256539503 |
| | | | [Source: HGNC Symbol; | | |
| | | | Acc: HGNC: 15511] | | |
| ENSG00000163395 | IGFN1 | HGNC Symbol | immunoglobulin-like and | −0.426568352 | 0.487780556 |
| | | | fibronectin type III | | |
| | | | domain containing 1 [Source: HGNC | | |
| | | | Symbol; Acc: HGNC: 24607] | | |
| ENSG00000163689 | C3orf67 | HGNC Symbol | chromosome 3 open reading frame 67 | −0.465955576 | 0.282307208 |
| | | | [Source: HGNC Symbol; | | |
| | | | Acc: HGNC: 24763] | | |
| ENSG00000163914 | RHO | HGNC Symbol | rhodopsin [Source: HGNC | −0.615499862 | 0.339797586 |
| | | | Symbol; Acc: HGNC: 10012] | | |
| ENSG00000163975 | MELTF | HGNC Symbol | melanotransferrin [Source: HGNC | −0.416776144 | 0.260507627 |
| | | | Symbol; Acc: HGNC: 7037] | | |
| ENSG00000163995 | ABLIM2 | HGNC Symbol | actio binding LIM protein | −0.520136403 | 0.063442539 |
| | | | family member 2 | | |
| | | | [Source: HGNC Symbol; | | |
| | | | Acc: HGNC: 19195] | | |
| ENSG00000164093 | PITX2 | HGNC Symbol | paired like homeodomain | −0.857868402 | 0.057313592 |
| | | | 2 [Source: HGNC | | |
| | | | Symbol; Acc: HGNC: 9005] | | |
| ENSG00000164107 | HAND2 | HGNC Symbol | heart and neural crest | −0.753644576 | 0.223874451 |
| | | | derivatives expressed 2 | | |
| | | | [Source: HGNC Symbol; | | |
| | | | Acc: HGNC: 4808] | | |
| ENSG00000164122 | ASB5 | HGNC Symbol | ankyrin repeat and SOCS | 0.079830425 | 0.915328605 |
| | | | box containing 5 | | |
| | | | [Source: HGNC Symbol; | | |
| | | | Acc: HGNC: 17180] | | |
| ENSG00000164265 | SCGB3A2 | HGNC Symbol | secretoglobin family 3A member 2 | −0.511511464 | 0.009388442 |
| | | | [Source: HGNC Symbol; | | |
| | | | Acc: HGNC: 18391] | | |
| ENSG00000164294 | GPX8 | HGNC Symbol | glutathione peroxidase 8 (putative) | −0.656518063 | 0.269654117 |
| | | | [Source: HGNC Symbol; | | |
| | | | Acc: HGNC: 33100] | | |
| ENSG00000164393 | ADGRF2 | HGNC Symbol | adhesion G protein-coupled | −0.3870263 | 0.551210043 |
| | | | receptor F2 | | |
| | | | [Source: HGNC Symbol; | | |
| | | | Acc: HGNC: 18991] | | |
| ENSG00000164692 | COLIA2 | HGNC Symbol | collagen type I alpha 2 | −1.329008825 | 0.002055954 |
| | | | chain [Source: HGNC | | |
| | | | Symbol; Acc: HGNC: 2198] | | |
| ENSG00000164694 | FNDC1 | HGNC Symbol | fibronectin type III | −0.656127373 | 0.211972787 |
| | | | domain containing 1 | | |
| | | | [Source: HGNC Symbol; | | |
| | | | Acc: HGNC: 21184] | | |
| ENSG00000164904 | ALDH7A1 | HGNC Symbol | aldehyde dehydrogenase | −0.490180368 | 0.202719915 |
| | | | 7 family member A1 | | |
| | | | [Source: HGNC Symbol; | | |
| | | | Acc: HGNC: 877] | | |
| ENSG00000165300 | SLITRK5 | HGNC Symbol | SLIT and NTRK like family member 5 | −0.675119097 | 0.028500813 |
| | | | [Source: HGNC Symbol; | | |
| | | | Acc: HGNC: 20295] | | |
| ENSG00000165323 | FAT3 | HGNC Symbol | FAT atypical cadherin | −0.385540325 | 0.505923328 |
| | | | 3 [Source: HGNC | | |
| | | | Symbol; Acc: HGNC: 23112] | | |
| ENSG00000165379 | LRFN5 | HGNC Symbol | leucine rich repeat and | −0.642769127 | 0.318351819 |
| | | | fibronectin type III | | |
| | | | domain containing 5 [Source: HGNC | | |
| | | | Symbol; Acc: HGNC: 20360] | | |
| ENSG00000165495 | PKNOX2 | HGNC Symbol | PGX/knotted 1 homeobox | −0.172065851 | 0.813551285 |
| | | | 2 [Source: HGNC | | |
| | | | Symbol; Acc: HGNC: 16714] | | |
| ENSG00000165566 | AMER2 | HGNC Symbol | APC membrane recruitment protein 2 | −0.383500908 | 0.550047859 |
| | | | [Source: HGNC Symbol; | | |
| | | | Acc: HGNC: 26360] | | |
| ENSG00000165757 | JCAD | HGNC Symbol | junctional cadherin 5 associated | −0.687536793 | 0.05310314 |
| | | | [Source: HGNC Symbol; | | |
| | | | Acc: HGNC: 23283] | | |
| ENSG00000165816 | VWA2 | HGNC Symbol | von Willebrand factor | −0.616958529 | 0.228018985 |
| | | | A domain containing 2 | | |
| | | | [Source: HGNC Symbol; | | |
| | | | Acc: HGNC: 24709] | | |
| ENSG00000165966 | PDZRN4 | HGNC Symbol | PDZ domain containing ring finger 4 | −0.710133651 | 0.283637569 |
| | | | [Source: HGNC Symbol; | | |
| | | | Acc: HGNC: 30552] | | |
| ENSG00000165970 | SLC6A5 | HGNC Symbol | solute carrier family 6 member 5 | −0.441556406 | 0.467030393 |
| | | | [Source: HGNC Symbol; | | |
| | | | Acc: HGNC: 11051] | | |
| ENSG00000165973 | NELL1 | HGNC Symbol | neural EGFL like 1 [Source: HGNC | −0.775652603 | 0.220165101 |
| | | | Symbol; Acc: HGNC: 7750] | | |
| ENSG00000166118 | SPATA19 | HGNC Symbol | spermatogenesis associated | −0.717800429 | 0.27315415 |
| | | | 19 [Source: HGNC | | |
| | | | Symbol; Acc: HGNC: 30614] | | |
| ENSG00000166159 | LRTM2 | HGNC Symbol | leucine rich repeats | −0.85369222 | 0.085585739 |
| | | | and transmembrane | | |
| | | | domains 2 [Source: HGNC | | |
| | | | Symbol; Acc: HGNC: 32443] | | |
| ENSG00000166292 | TMEM100 | HGNC Symbol | transmembrane protein | −0.250984168 | 0.758168139 |
| | | | 100 [Source: HGNC | | |
| | | | Symbol; Acc: HGNC: 25607] | | |
| ENSG00000166391 | MDGAT2 | HGNC Symbol | monoacylglycerol O-acyltransferase 2 | −0.648731495 | 0.319053284 |
| | | | [Source: HGNC Symbol; | | |
| | | | Acc: HGNC: 23248] | | |
| ENSG00000166444 | ST5 | HGNC Symbol | suppression of tumorigenicity | −0.623988928 | 0.08234925 |
| | | | 5 [Source: HGNC | | |
| | | | Symbol; Acc: HGNC: 11350] | | |
| ENSG00000166473 | PKD1L2 | HGNC Symbol | polycystin 1 like 2 | −0.290071825 | 0.668311557 |
| | | | (gene/pseudogene) | | |
| | | | [Source: HGNC Symbol; | | |
| | | | Acc: HGNC: 21715] | | |
| ENSG00000166558 | SLC38A8 | HGNC Symbol | solute carrier family | −0.565390451 | 0.349528978 |
| | | | 38 member 8 | | |
| | | | [Source: HGNC Symbol; | | |
| | | | Acc: HGNC: 32434] | | |
| ENSG00000166596 | CFAP52 | HGNC Symbol | cilia and flagella associated | −0.373333127 | 0.567527075 |
| | | | protein 52 | | |
| | | | [Source: HGNC Symbol; | | |
| | | | Acc: HGNC: 16053] | | |
| ENSG00000166689 | PLEKHA7 | HGNC Symbol | pleckstrin homology domain | −0.295527396 | 0.24002465 |
| | | | containing A7 | | |
| | | | [Source: HGNC Symbol; | | |
| | | | Acc: HGNC: 27049] | | |
| ENSG00000166863 | TAC3 | HGNC Symbol | tachykinin 3 [Source: HGNC | −0.725563817 | 0.198940702 |
| | | | Symbol; Acc: HGNC: 11521] | | |
| ENSG00000166869 | CHP2 | HGNC Symbol | calcineurin like EF-hand protein 2 | −0.947768021 | 0.112210394 |
| | | | [Source: HGNC Symbol; | | |
| | | | Acc: HGNC: 24927] | | |
| ENSG00000166984 | TCP10L2 | HGNC Symbol | t-complex 10 like 2 [Source: HGNC | −0.754572409 | 0.150977424 |
| | | | Symbol; Acc: HGNC: 21254] | | |
| ENSG00000167654 | ATCAY | HGNC Symbol | “ATCAY, caytaxin [Source: HGNC | −0.90959213 | 0.089473968 |
| | | | Symbol; Acc: HGNC: 779]” | | |
| ENSG00000167723 | TRPV3 | HGNC Symbol | transient receptor potential | −0.563018449 | 0.047202728 |
| | | | cation channel | | |
| | | | subfamily V member 3 [Source: HGNC | | |
| | | | Symbol; Acc: HGNC: 18084] | | |
| ENSG00000167757 | KLK11 | HGNC Symbol | kallikrein related peptidase 11 | −0.877178887 | 0.126229199 |
| | | | [Source: HGNC Symbol; | | |
| | | | Acc: HGNC: 6359] | | |
| ENSG00000167779 | IGFBP6 | HGNC Symbol | insulin like growth factor | −0.44480682 | 0.307541999 |
| | | | binding protein 6 | | |
| | | | [Source: HGNC Symbol; | | |
| | | | Acc: HGNC: 5475] | | |
| ENSG00000167798 | C3P1 | HGNC Symbol | complement component 3 | −0.397404693 | 0.575036428 |
| | | | precursor pseudogene | | |
| | | | [Source: HGNC Symbol; | | |
| | | | Acc: HGNC: 34414] | | |
| ENSG00000168077 | SCARA3 | HGNC Symbol | scavenger receptor class | −0.633708326 | 0.238072641 |
| | | | A member 3 | | |
| | | | [Source: HGNC Symbol; | | |
| | | | Acc: HGNC: 19000] | | |
| ENSG00000168079 | SCARA5 | HGNC Symbol | scavenger receptor class | −0.678863321 | 0.111887496 |
| | | | A member 5 | | |
| | | | [Source: HGNC Symbol; | | |
| | | | Acc: HGNC: 28701] | | |
| ENSG00000168356 | SCN11A | HGNC Symbol | sodium voltage-gated | −0.418872904 | 0.366781722 |
| | | | channel alpha subunit 11 | | |
| | | | [Source: HGNC Symbol; | | |
| | | | Acc: HGNC: 10583] | | |
| ENSG00000168367 | LINC00917 | HGNC Symbol | long intergenic non-protein | −0.11663907 | 0.89274309 |
| | | | coding RNA 917 | | |
| | | | [Source: HGNC Symbol; | | |
| | | | Acc: HGNC: 48607] | | |
| ENSG00000168481 | LGI3 | HGNC Symbol | leucine rich repeat LGI | −0.573256523 | 0.302289133 |
| | | | family member 3 | | |
| | | | [Source: HGNC Symbol; | | |
| | | | Acc: HGNC: 18711] | | |
| ENSG00000168484 | SFTPC | HGNC Symbol | surfactant protein | −1.037382046 | 0.063132935 |
| | | | C [Source: HGNC | | |
| | | | Symbol; Acc: HGNC: 10802] | | |
| ENSG00000168542 | COL3A1 | HGNC Symbol | collagen type III alpha 1 chain | −1.474761932 | 0.012911146 |
| | | | [Source: HGNC Symbol; | | |
| | | | Acc: HGNC: 2201] | | |
| ENSG00000168907 | PLA2G4F | HGNC Symbol | phospholipase A2 group | −0.587140187 | 0.299414708 |
| | | | IVF [Source: HGNC | | |
| | | | Symbol; Acc: HGNC: 27396] | | |
| ENSG00000168959 | GRM5 | HGNC Symbol | glutamate metabotropic receptor 5 | −0.453630369 | 0.440906003 |
| | | | [Source: HGNC Symbol; | | |
| | | | Acc: HGNC: 4597] | | |
| ENSG00000169126 | ARMC4 | HGNC Symbol | armadillo repeat containing | −0.575622675 | 0.299414708 |
| | | | 4 [Source: HGNC | | |
| | | | Symbol; Acc: HGNC: 25583] | | |
| ENSG00000169169 | CPT1C | HGNC Symbol | carnitine palmitoyltransferase 1C | −0.341160496 | 0.476106903 |
| | | | [Source: HGNC Symbol; | | |
| | | | Acc: HGNC: 18540] | | |
| ENSG00000169344 | UMDD | HGNC Symbol | uromodulin [Source: HGNC | −0.76971558 | 0.181197865 |
| | | | Symbol; Acc: HGNC: 12559] | | |
| ENSG00000169436 | COL22A1 | HGNC Symbol | collagen type XXII alpha 1 chain | −0.374497855 | 0.528626826 |
| | | | [Source: HGNC Symbol; | | |
| | | | Acc: HGNC: 22989] | | |
| ENSG00000169862 | CTNND2 | HGNC Symbol | catenin delta 2 [Source: HGNC | −0.350904798 | 0.520102355 |
| | | | Symbol; Acc: HGNC: 2516] | | |
| ENSG00000169876 | MUC17 | HGNC Symbol | “mucin 17, cell | −0.566443746 | 0.387767501 |
| | | | surface associated | | |
| | | | [Source: HGNC Symbol; | | |
| | | | Acc: HGNC: 16800]” | | |
| ENSG00000170381 | SEMA3E | HGNC Symbol | semaphorin 3E [Source: HGNC | −0.598736051 | 0.385408727 |
| | | | Symbol; Acc: HGNC: 10727] | | |
| ENSG00000170426 | SDR9C7 | HGNC Symbol | short chain dehydragenase/ | −0.869641519 | 0.163679446 |
| | | | reductase family 9C | | |
| | | | member 7 [Source: HGNC | | |
| | | | Symbol; Acc: HGNC: 29958] | | |
| ENSG00000170442 | KRT86 | HGNC Symbol | keratin 86 [Source: HGNC | −0.370773529 | 0.370892028 |
| | | | Symbol; Acc: HGNC: 6463] | | |
| ENSG00000170703 | TTLL6 | HGNC Symbol | tubulin tyrosine ligase like 6 | −0.589944567 | 0.344781215 |
| | | | [Source: HGNC Symbol; | | |
| | | | Acc: HGNC: 26664] | | |
| ENSG00000170777 | TPD52L3 | HGNC Symbol | tumor protein D52 like | −0.585215371 | 0.382906193 |
| | | | 3 [Source: HGNC | | |
| | | | Symbol; Acc: HGNC: 23382] | | |
| ENSG00000170927 | PKHD1 | HGNC Symbol | “PKHD1, fibrocystin/polyductin | −0.246967015 | 0.708015783 |
| | | | [Source: HGNC Symbol; | | |
| | | | Acc: HGNC: 9016]” | | |
| ENSG00000171435 | KSR2 | HGNC Symbol | kinase suppressor of | −0.428105876 | 0.450661319 |
| | | | ras 2 [Source: HGNC | | |
| | | | Symbol; Acc: HGNC: 18610] | | |
| ENSG00000171487 | NLRP5 | HGNC Symbol | NLR family pyrin domain containing 5 | −0.238462882 | 0.710453093 |
| | | | [Source: HGNC Symbol; | | |
| | | | Acc: HGNC: 21269] | | |
| ENSG00000171533 | MAP6 | HGNC Symbol | microtubule associated protein G | −0.731727753 | 0.156310461 |
| | | | [Source: HGNC Symbol; | | |
| | | | Acc: HGNC: 6868] | | |
| ENSG00000171564 | FGB | HGNC Symbol | fibrinogen beta chain [Source: HGNC | −0.538885417 | 0.363987255 |
| | | | Symbol; Acc: HGNC: 3662] | | |
| ENSG00000171587 | DSCAM | HGNC Symbol | DS cell adhesion molecule | −0.379128207 | 0.536270648 |
| | | | [Source: HGNC | | |
| | | | Symbol; Acc: HGNC: 3039] | | |
| ENSG00000171804 | WDR87 | HGNC Symbol | WD repeat domain 87 [Source: HGNC | −0.521912283 | 0.382145668 |
| | | | Symbol; Acc: HGNC: 29934] | | |
| ENSG00000172137 | CALB2 | HGNC Symbol | calbindin 2 [Source: HGNC | −0.440233971 | 0.434741137 |
| | | | Symbol; Acc: HGNC: 1435] | | |
| ENSG00000172350 | ABCG4 | HGNC Symbol | ATP binding cassette | −0.409715093 | 0.501052173 |
| | | | subfamily G member 4 | | |
| | | | [Source: HGNC Symbol; | | |
| | | | Acc: HGNC: 13884] | | |
| ENSG00000172752 | COL6A5 | HGNC Symbol | collagen type VI alpha 5 chain | −0.36150757 | 0.544869606 |
| | | | [Source: HGNC Symbol; | | |
| | | | Acc: HGNC: 26674] | | |
| ENSG00000172900 | AP002387.1 | Clone-based | | −0.667266117 | 0.284084703 |
| | | (Ensembl) gene | | | |
| ENSG00000172995 | ARPP21 | HGNC Symbol | cAMP regulated phosphoprotein 21 | −0.416436637 | 0.360871156 |
| | | | [Source: HGNC Symbol; | | |
| | | | Acc: HGNC: 16968] | | |
| ENSG00000173013 | CCDC96 | HGNC Symbol | coiled-coil domain containing 96 | −0.507224369 | 0.111947151 |
| | | | [Source: HGNC Symbol; | | |
| | | | Acc: HGNC: 26900] | | |
| ENSG00000173227 | SYT12 | HGNC Symbol | synaptotagmin 12 [Source: HGNC | −0.752963578 | 0.176041345 |
| | | | Symbol; Acc: HGNC: 18381] | | |
| ENSG00000173572 | NLRP13 | HGNC Symbol | NLR family pyrin | −0.337498608 | 0.604591273 |
| | | | domain containing 13 | | |
| | | | [Source: HGNC Symbol; | | |
| | | | Acc: HGNC: 22937] | | |
| ENSG00000173702 | MUC13 | HGNC Symbol | “mucin 13, cell surface associated | −0.770938778 | 0.184683607 |
| | | | [Source: HGNC Symbol; | | |
| | | | Acc: HGNC: 7511]” | | |
| ENSG00000173769 | TOPAZ1 | HGNC Symbol | testis and ovary specific | −0.581049747 | 0.337975386 |
| | | | PAZ domain containing 1 | | |
| | | | [Source: HGNC Symbol; | | |
| | | | Acc: HGNC: 24746] | | |
| ENSG00000173826 | KCNH6 | HGNC Symbol | potassium voltage-gated | −0.668262944 | 0.228310769 |
| | | | channel subfamily H | | |
| | | | member 6 [Source: HGNC | | |
| | | | Symbol; Acc: HGNC: 18862] | | |
| ENSG00000174279 | EVX2 | HGNC Symbol | even-skipped homeobox | −0.532607087 | 0.366352883 |
| | | | 2 [Source: HGNC | | |
| | | | Symbol; Acc: HGNC: 3507] | | |
| ENSG00000174358 | SLC6A19 | HGNC Symbol | solute carrier family G member 19 | −0.552428142 | 0.229701299 |
| | | | [Source: HGNC Symbol; | | |
| | | | Acc: HGNC: 27960] | | |
| ENSG00000174498 | IGDCC3 | HGNC Symbol | immunoglobulin superfamily | −0.500416529 | 0.386556 |
| | | | DCC subclass | | |
| | | | member 3 [Source: HGNC | | |
| | | | Symbol; Acc: HGNC: 9700] | | |
| ENSG00000174502 | SLC26A9 | HGNC Symbol | solute carrier family 26 member 9 | −0.707160365 | 0.159137172 |
| | | | [Source: HGNC Symbol; | | |
| | | | Acc: HGNC: 14469] | | |
| ENSG00000174963 | ZIC4 | HGNC Symbol | Zic family member 4 [Source: HGNC | −0.741912248 | 0.199304778 |
| | | | Symbol; Acc: HGNC: 20393] | | |
| ENSG00000175084 | DES | HGNC Symbol | desmin [Source: HGNC Symbol; | −0.74969534 | 0.040474867 |
| | | | Acc: HGNC: 2770] | | |
| ENSG00000175267 | VWA3A | HGNC Symbol | von Willebrand factor A | −0.713694352 | 0.037008563 |
| | | | domain containing 3A | | |
| | | | [Source: HGNC Symbol; | | |
| | | | Acc: HGNC: 27088] | | |
| ENSG00000175267 | VWA3A | HGNC Symbol | von Willebrand factor A | −0.713694352 | 0.037008563 |
| | | | domain containing 3A | | |
| | | | [Source: HGNC Symbol; | | |
| | | | Acc: HGNC: 27088] | | |
| ENSG00000175329 | ISX | HGNC Symbol | intestine specific homeobox | −0.48812621 | 0.42503419 |
| | | | [Source: HGNC | | |
| | | | Symbol; Acc: HGNC: 28084] | | |
| ENSG00000175535 | PNLIP | HGNC Symbol | pancreatic lipase [Source: HGNC | −0.830503663 | 0.166474844 |
| | | | Symbol; Acc: HGNC: 9155] | | |
| ENSG00000176584 | DMBT1P1 | HGNC Symbol | deleted in malignant brain | −0.301771252 | 0.660657448 |
| | | | tumors 1 pseudogene 1 | | |
| | | | [Source: HGNC Symbol; | | |
| | | | Acc: HGNC: 49497] | | |
| ENSG00000176769 | TCERG1L | HGNC Symbol | transcription elongation | −0.433973597 | 0.471307255 |
| | | | regulator 1 like | | |
| | | | [Source: HGNC Symbol; | | |
| | | | Acc: HGNC: 23533] | | |
| ENSG00000177045 | SIX5 | HGNC Symbol | SIX homeobox 5 [Source: HGNC | −0.498221295 | 0.307541999 |
| | | | Symbol; Acc: HGNC: 10891] | | |
| ENSG00000177103 | DSCAML1 | HGNC Symbol | DS cell adhesion molecule like 1 | −0.501025251 | 0.288065754 |
| | | | [Source: HGNC Symbol; | | |
| | | | Acc: HGNC: 14656] | | |
| ENSG00000177354 | C10orf71 | HGNC Symbol | chromosome 10 open reading frame 71 | −0.437224218 | 0.462998721 |
| | | | [Source: HGNC Symbol; | | |
| | | | Acc: HGNC: 26973] | | |
| ENSG00000177511 | ST8SIA3 | HGNC Symbol | “ST8 alpha-N-acetyl-neuraminide | −0.634752306 | 0.292333593 |
| | | | alpha-2,8-sialyltransferase | | |
| | | | 3 [Source: HGNC | | |
| | | | Symbol; Acc: HGNC: 14269]” | | |
| ENSG00000177551 | NHLH2 | HGNC Symbol | nescient helix-loop-helix | −0.492445704 | 0.413985661 |
| | | | 2 [Source: HGNC | | |
| | | | Symbol; Acc: HGNC: 7818] | | |
| ENSG00000178031 | ADAMTSL1 | HGNC Symbol | ADAMTS like 1 [Source: HGNC | −0.351255676 | 0.529035355 |
| | | | Symbol; Acc: HGNC: 14632] | | |
| ENSG00000178171 | AMER3 | HGNC Symbol | APC membrane recruitment protein 3 | −0.457777165 | 0.432259224 |
| | | | [Source: HGNC Symbol; | | |
| | | | Acc: HGNC: 26771] | | |
| ENSG00000178568 | ERBB4 | HGNC Symbol | erb-b2 receptor tyrosine kinase 4 | −0.165545558 | 0.837547319 |
| | | | [Source: HGNC Symbol; | | |
| | | | Acc: HGNC: 3432] | | |
| ENSG00000178645 | C10orf53 | HGNC Symbol | chromosome 10 open reading frame 53 | −0.503971682 | 0.476649736 |
| | | | [Source: HGNC Symbol; | | |
| | | | Acc: HGNC: 27421] | | |
| ENSG00000178965 | ERICH3 | HGNC Symbol | glutamate rich 3 [Source: HGNC | −0.468692004 | 0.391350792 |
| | | | Symbol; Acc: HGNC: 25346] | | |
| ENSG00000179008 | C14orf39 | HGNC Symbol | chromosome 14 open reading frame 39 | −0.402370572 | 0.557284045 |
| | | | [Source: HGNC Symbol; | | |
| | | | Acc: HGNC: 19849] | | |
| ENSG00000179178 | TMEM125 | HGNC Symbol | transmembrane protein | −0.507984532 | 0.427803498 |
| | | | 125 [Source: HGNC | | |
| | | | Symbol; Acc: HGNC: 28275] | | |
| ENSG00000179270 | C2orf71 | HGNC Symbol | chromosome 2 open reading frame 71 | −0.18942048 | 0.758600189 |
| | | | [Source: HGNC Symbol; | | |
| | | | Acc: HGNC: 34383] | | |
| ENSG00000179709 | NLRP8 | HGNC Symbol | NLR family pyrin | −0.380100885 | 0.555880858 |
| | | | domain containing 8 | | |
| | | | [Source: HGNC Symbol; | | |
| | | | Acc: HGNC: 22940] | | |
| ENSG00000179766 | ATP8B5P | HGNC Symbol | “ATPase phospholipid | −1.080551014 | 0.010971767 |
| | | | transporting 8B5, | | |
| | | | pseudogene [Source: HGNC | | |
| | | | Symbol; Acc: HGNC: 27245]” | | |
| ENSG00000179813 | FAM216B | HGNC Symbol | family with sequence | −0.305849787 | 0.706075351 |
| | | | similarity 216 member B | | |
| | | | [Source: HGNC Symbol; | | |
| | | | Acc: HGNC: 26883] | | |
| ENSG00000180251 | SLC9A4 | HGNC Symbol | solute carrier family 9 member A4 | −0.349423486 | 0.580620411 |
| | | | [Source: HGNC Symbol; | | |
| | | | Acc: HGNC: 11077] | | |
| ENSG00000181143 | MUC16 | HGNC Symbol | “mucin 16, cell | −0.329348592 | 0.430361664 |
| | | | surface associated | | |
| | | | [Source: HGNC Symbol; | | |
| | | | Acc: HGNC: 15582]” | | |
| ENSG00000181355 | DFCC1 | HGNC Symbol | orofacial cleft 1 candidate | −0.532362428 | 0.437641901 |
| | | | 1 [Source: HGNC | | |
| | | | Symbol; Acc: HGNC: 21017] | | |
| ENSG00000181378 | CFAP65 | HGNC Symbol | cilia and flagella associated | −0.434847926 | 0.442646097 |
| | | | protein 65 | | |
| | | | [Source: HGNC Symbol; | | |
| | | | Acc: HGNC: 25325] | | |
| ENSG00000182256 | GABRG3 | HGNC Symbol | gamma-aminobutyric | −0.256293297 | 0.696478261 |
| | | | acid type A receptor | | |
| | | | gamma3 subunit [Source: HGNC | | |
| | | | Symbol; Acc: HGNC: 4088] | | |
| ENSG00000183016 | NA | NA | NA | −0.30865823 | 0.660441616 |
| ENSG00000183067 | IGSF5 | HGNC Symbol | immunoglobulin superfamily member 5 | −0.51302971 | 0.457242584 |
| | | | [Source: HGNC Symbol; | | |
| | | | Acc: HGNC: 5952] | | |
| ENSG00000183206 | POTEC | HGNC Symbol | POTE ankyrin domain family member C | −0.327817046 | 0.614566385 |
| | | | [Source: HGNC Symbol; | | |
| | | | Acc: HGNC: 33894] | | |
| ENSG00000183242 | WT1-AS | HGNC Symbol | WT1 antisense RNA [Source: HGNC | −0.327487007 | 0.63530305 |
| | | | Symbol; Acc: HGNC: 18135] | | |
| ENSG00000183287 | CCBE1 | HGNC Symbol | collagen and calcium binding | −0.590879294 | 0.238072641 |
| | | | EGF domains 1 | | |
| | | | [Source: HGNC Symbol; | | |
| | | | Acc: HGNC: 29426] | | |
| ENSG00000183304 | FAM9A | HGNC Symbol | family with sequence | −0.938058237 | 0.096563675 |
| | | | similarity 9 member A | | |
| | | | [Source: HGNC Symbol; | | |
| | | | Acc: HGNC: 18403] | | |
| ENSG00000183317 | EPHA10 | HGNC Symbol | EPH receptor A10 [Source: HGNC | −0.469692297 | 0.372911095 |
| | | | Symbol; Acc: HGNC: 19987] | | |
| ENSG00000183580 | FBXL7 | HGNC Symbol | F-box and leucine rich | −0.591833997 | 0.365077842 |
| | | | repeat protein 7 | | |
| | | | [Source: HGNC Symbol; | | |
| | | | Acc: HGNC: 13604] | | |
| ENSG00000183668 | PSG9 | HGNC Symbol | pregnancy specific | −0.781212682 | 0.219746846 |
| | | | bata-1-glycoprotein 9 | | |
| | | | [Source: HGNC Symbol; | | |
| | | | Acc: HGNC: 9526] | | |
| ENSG00000183778 | B3GALT5 | HGNC Symbol | “bata-1,3- | −0.49369755 | 0.401927772 |
| | | | galactosyltransfarase 5 | | |
| | | | [Source: HGNC Symbol; | | |
| | | | Acc: HGNC: 920]” | | |
| ENSG00000183780 | SLC35F3 | HGNC Symbol | solute carrier family 35 member F3 | −0.289076693 | 0.422151105 |
| | | | [Source: HGNC Symbol; | | |
| | | | Acc: HGNC: 23616] | | |
| ENSG00000183856 | IQGAP3 | HGNC Symbol | IQ motif containing GTPase | −0.465335464 | 0.302087891 |
| | | | activating protein 3 | | |
| | | | [Source: HGNC Symbol; | | |
| | | | Acc: HGNC: 20669] | | |
| ENSG00000183876 | ARS1 | HGNC Symbol | arylsulfatase family member 1 | −0.487563606 | 0.409216161 |
| | | | [Source: HGNC Symbol; | | |
| | | | Acc: HGNC: 32521] | | |
| ENSG00000183908 | LRRC55 | HGNC Symbol | leucine rich repeat containing 55 | −0.306233165 | 0.604776413 |
| | | | [Source: HGNC Symbol; | | |
| | | | Acc: HGNC: 32324] | | |
| ENSG00000184012 | TMPRSS2 | HGNC Symbol | transmembrane serine protease 2 | −0.918034548 | 0.037008563 |
| | | | [Source: HGNC Symbol; | | |
| | | | Acc: HGNC: 11876] | | |
| ENSG00000184227 | ACOT1 | HGNC Symbol | acyl-CoA thioesterase | −0.665299244 | 0.162555375 |
| | | | 1 [Source: HGNC | | |
| | | | Symbol; Acc: HGNC: 33128] | | |
| ENSG00000184302 | SIX6 | HGNC Symbol | SIX homeobox 6 [Source: HGNC | −0.504088891 | 0.441451067 |
| | | | Symbol; Acc: HGNC: 10892] | | |
| ENSG00000184304 | PRKD1 | HGNC Symbol | protein kinase D1 [Source: HGNC | −0.428587371 | 0.415595988 |
| | | | Symbol; Acc: HGNC: 9407] | | |
| ENSG00000184809 | B3GALT5-AS1 | HGNC Symbol | B3GALT5 antisense | −0.338454269 | 0.614566385 |
| | | | RNA1 [Source: HGNC | | |
| | | | Symbol; Acc: HGNC: 16424] | | |
| ENSG00000184908 | CLCNKB | HGNC Symbol | chloride voltage-gated channel Kb | −0.357681839 | 0.540451079 |
| | | | [Source: HGNC Symbol; | | |
| | | | Acc: HGNC: 2027] | | |
| ENSG00000185038 | MRDH2A | HGNC Symbol | maestro heat like repeat | −0.38431913 | 0.523839641 |
| | | | family member 2A | | |
| | | | [Source: HGNC Symbol; | | |
| | | | Acc: HGNC: 27936] | | |
| ENSG00000185069 | KRT7B | HGNC Symbol | keratin 76 [Source: HGNC | −0.5580061 | 0.363389835 |
| | | | Symbol; Acc: HGNC: 24430] | | |
| ENSG00000185303 | SFTPA2 | HGNC Symbol | surfactant protein | −0.803359583 | 0.189636469 |
| | | | A2 [Source: HGNC | | |
| | | | Symbol; Acc: HGNC: 10799] | | |
| ENSG00000185313 | SCN10A | HGNC Symbol | sodium voltage-gated channel | −0.230551689 | 0.73625037 |
| | | | alpha subunit 10 | | |
| | | | [Source: HGNC Symbol; | | |
| | | | Acc: HGNC: 10582] | | |
| ENSG00000185467 | KPNA7 | HGNC Symbol | karyapherin subunit alpha | −0.199440359 | 0.827997131 |
| | | | 7 [Source: HGNC | | |
| | | | Symbol; Acc: HGNC: 21839] | | |
| ENSG00000185686 | PRAME | HGNC Symbol | preferentially expressed | −0.683189263 | 0.231379249 |
| | | | antigen in melanome | | |
| | | | [Source: HGNC Symbol; | | |
| | | | Acc: HGNC: 9336] | | |
| ENSG00000185737 | NRG3 | HGNC Symbol | neuregulin 3 [Source: HGNC | −0.331707231 | 0.60838718 |
| | | | Symbol; Acc: HGNC: 7999] | | |
| ENSG00000185739 | SRL | HGNC Symbol | sarcalumenin [Source: HGNC | −0.778970248 | 0.182933474 |
| | | | Symbol; Acc: HGNC: 11295] | | |
| ENSG00000185823 | NPAP1 | HGNC Symbol | nuclear pore associated protein 1 | −0.709460544 | 0.239778807 |
| | | | [Source: HGNC Symbol; | | |
| | | | Acc: HGNC: 1190] | | |
| ENSG00000185974 | GRK1 | HGNC Symbol | G protein-coupled receptor kinase 1 | −0.524927614 | 0.365633153 |
| | | | [Source: HGNC Symbol; | | |
| | | | Acc: HGNC: 10013] | | |
| ENSG00000186185 | KIF18B | HGNC Symbol | kinesin family member | −0.533902601 | 0.08735537 |
| | | | 18B [Source: HGNC | | |
| | | | Symbol; Acc: HGNC: 27102] | | |
| ENSG00000186393 | KRT2G | HGNC Symbol | keratin 26 [Source: HGNC | −0.510960904 | 0.393531656 |
| | | | Symbol; Acc: HGNC: 30840] | | |
| ENSG00000186487 | MYT1L | HGNC Symbol | myelin transcription factor 1 like | −0.551259753 | 0.323477526 |
| | | | [Source: HGNC Symbol; | | |
| | | | Acc: HGNC: 7623] | | |
| ENSG00000186526 | CYP4F8 | HGNC Symbol | cytochrome P450 family 4 | −0.633956341 | 0.284297086 |
| | | | subfamily F member 8 | | |
| | | | [Source: HGNC Symbol; | | |
| | | | Acc: HGNC: 2648] | | |
| ENSG00000186862 | PDZD7 | HGNC Symbol | PDZ domain containing | −0.554675047 | 0.164105126 |
| | | | 7 [Source: HGNC | | |
| | | | Symbol; Acc: HGNC: 26257] | | |
| ENSG00000187068 | C3orf70 | HGNC Symbol | chromosome 3 open | −0.188322704 | 0.736814103 |
| | | | reading frame 70 | | |
| | | | [Source: HGNC Symbol; | | |
| | | | Acc: HGNC: 33731] | | |
| ENSG00000187094 | DDK | HGNC Symbol | cholecystokinin [Source: HGNC | −0.809919388 | 0.141982169 |
| | | | Symbol; Acc: HGNC: 1569] | | |
| ENSG00000187123 | LYPDG | HGNC Symbol | LYG/PLAUR domain containing 6 | −0.509808838 | 0.401011395 |
| | | | [Source: HGNC Symbol; | | |
| | | | Acc: HGNC: 28751] | | |
| ENSG00000187527 | ATP13A5 | HGNC Symbol | ATPase 13A5 [Source: HGNC | −0.467545909 | 0.428173196 |
| | | | Symbol; Acc: HGNC: 31789] | | |
| ENSG00000187772 | LIN28B | HGNC Symbol | lin-28 homolog B [Source: HGNC | −0.23447087 | 0.763770257 |
| | | | Symbol; Acc: HGNC: 32207] | | |
| ENSG00000187905 | LRRC74B | HGNC Symbol | leucine rich repeat containing 74B | −0.461043362 | 0.41745351 |
| | | | [Source: HGNC Symbol; | | |
| | | | Acc: HGNC: 34301] | | |
| ENSG00000187955 | COL14A1 | HGNC Symbol | collagen type XIV alpha 1 chain | −0.802642614 | 0.060883822 |
| | | | [Source: HGNC Symbol; | | |
| | | | Acc: HGNC: 2191] | | |
| ENSG00000187997 | C17orf99 | HGNC Symbol | chromosome 17 open reading frame 99 | −0.54593757 | 0.228439083 |
| | | | [Source: HGNC Symbol; | | |
| | | | Acc: HGNC: 34490] | | |
| ENSG00000188086 | PRSS45 | HGNC Symbol | serine protease 45 [Source: HGNC | −0.561060502 | 0.385562603 |
| | | | Symbol; Acc: HGNC: 30717] | | |
| ENSG00000188112 | C6orf132 | HGNC Symbol | chromosome 6 open reading frame 132 | −0.912928908 | 0.01832644 |
| | | | [Source: HGNC Symbol; | | |
| | | | Acc: HGNC: 21288] | | |
| ENSG00000188162 | OTOG | HGNC Symbol | otogelin [Source: HGNC Symbol; | −0.501929759 | 0.346681837 |
| | | | Acc: HGNC: 8516] | | |
| ENSG00000188338 | SLC38A3 | HGNC Symbol | solute carrier family 38 member 3 | −0.761738167 | 0.152208524 |
| | | | [Source: HGNC Symbol; | | |
| | | | Acc: HGNC: 18044] | | |
| ENSG00000188488 | SERPINA5 | HGNC Symbol | serpin family A member | −0.692734776 | 0.249894109 |
| | | | 5 [Source: HGNC | | |
| | | | Symbol; Acc: HGNC: 8723] | | |
| ENSG00000188706 | ZDHHC9 | HGNC Symbol | zinc finger DHHC-type containing 9 | −0.385110334 | 0.030930967 |
| | | | [Source: HGNC Symbol; | | |
| | | | Acc: HGNC: 18475] | | |
| ENSG00000188782 | DATSPER4 | HGNC Symbol | cation channel sperm associated 4 | −0.829398746 | 0.157963233 |
| | | | [Source: HGNC Symbol; | | |
| | | | Acc: HGNC: 23220] | | |
| ENSG00000188803 | SHISA6 | HGNC Symbol | shisa family member 6 [Source: HGNC | −0.516219657 | 0.41821783 |
| | | | Symbol; Acc: HGNC: 34491] | | |
| ENSG00000188886 | ASTL | HGNC Symbol | astacin like metalloendopeptidase | −1.137626195 | 0.005079261 |
| | | | [Source: HGNC Symbol; | | |
| | | | Acc: HGNC: 31704] | | |
| ENSG00000196091 | MYBPD1 | HGNC Symbol | “myosin binding protein | −0.430673258 | 0.437393542 |
| | | | C, slow type | | |
| | | | [Source: HGNC Symbol; | | |
| | | | Acc: HGNC: 7549]” | | |
| ENSG00000196136 | SERPINA3 | HGNC Symbol | serpin family A member | −0.909069758 | 0.106147674 |
| | | | 3 [Source: HGNC | | |
| | | | Symbol; Acc: HGNC: 16] | | |
| ENSG00000196361 | ELAVL3 | HGNC Symbol | ELAV like RNA binding protein 3 | −0.75893849 | 0.141285189 |
| | | | [Source: HGNC Symbol; | | |
| | | | Acc: HGNC: 3314] | | |
| ENSG00000196415 | PRTN3 | HGNC Symbol | proteinase 3 [Source: HGNC | −1.443754633 | 0.002256568 |
| | | | Symbol; Acc: HGNC: 9495] | | |
| ENSG00000196565 | HBG2 | HGNC Symbol | hemoglobin subunit gamma | −0.931424276 | 0.014195699 |
| | | | 2 [Source: HGNC | | |
| | | | Symbol; Acc: HGNC: 4832] | | |
| ENSG00000196862 | RGPD4 | HGNC Symbol | RANBP2-like and GRIP | −0.694081274 | 0.03331637 |
| | | | domain containing 4 | | |
| | | | [Source: HGNC Symbol; | | |
| | | | Acc: HGNC: 32417] | | |
| ENSG00000197079 | KRT35 | HGNC Symbol | keratin 35 [Source: HGNC | −0.658605324 | 0.248428762 |
| | | | Symbol; Acc: HGNC: 6453] | | |
| ENSG00000197085 | NPSR1-AS1 | HGNC Symbol | NPSR1 antisense RNA | −0.47682751 | 0.385383626 |
| | | | 1 [Source: HGNC | | |
| | | | Symbol; Acc: HGNC: 22128] | | |
| ENSG00000197406 | DIO3 | HGNC Symbol | iodothyronine deiodinase | −0.582483697 | 0.278225713 |
| | | | 3 [Source: HGNC | | |
| | | | Symbol; Acc: HGNC: 2885] | | |
| ENSG00000197408 | CYP2BG | HGNC Symbol | cytochrome P450 family 2 | −0.59980912 | 0.385010224 |
| | | | subfamily B member | | |
| | | | 6 [Source: HGNC Symbol; | | |
| | | | Acc: HGNC: 2615] | | |
| ENSG00000197893 | NRAP | HGNC Symbol | neublin related anchoring protein | −0.32566366 | 0.63782084 |
| | | | [Source: HGNC Symbol; | | |
| | | | Acc: HGNC: 7988] | | |
| ENSG00000197991 | AL592490.1 | Clone-based | | −0.605428219 | 0.343100516 |
| | | (Ensembl) gene | | | |
| ENSG00000198010 | DLGAP2 | HGNC Symbol | DLG associated protein | −0.462914925 | 0.409741714 |
| | | | 2 [Source: HGNC | | |
| | | | Symbol; Acc: HGNC: 2906] | | |
| ENSG00000198597 | ZNF536 | HGNC Symbol | zinc finger protein | −0.318101569 | 0.612448109 |
| | | | 536 [Source: HGNC | | |
| | | | Symbol; Acc: HGNC: 29025] | | |
| ENSG00000198765 | SYCP1 | HGNC Symbol | synaptonemal complex | −0.4742583 | 0.465583509 |
| | | | protein 1 [Source: HGNC | | |
| | | | Symbol; Acc: HGNC: 11487] | | |
| ENSG00000198788 | MUC2 | HGNC Symbol | “mucin 2, oligomeric | −0.356584229 | 0.524732504 |
| | | | mucus/gel-forming | | |
| | | | [Source: HGNC Symbol; | | |
| | | | Acc: HGNC: 7512]” | | |
| ENSG00000198914 | POU3F3 | HGNC Symbol | POU class 3 homeobox | −0.566373551 | 0.326225195 |
| | | | 3 [Source: HGNC | | |
| | | | Symbol; Acc: HGNC: 9216] | | |
| ENSG00000198929 | NDS1AP | HGNC Symbol | citric oxide synthase | −0.522447538 | 0.23360559 |
| | | | 1 adaptor protein | | |
| | | | [Source: HGNC Symbol; | | |
| | | | Acc: HGNC: 16859] | | |
| ENSG00000203805 | PLPP4 | HGNC Symbol | phospholipid phosphatase | −0.645867767 | 0.226621278 |
| | | | 4 [Source: HGNC | | |
| | | | Symbol; Acc: HGNC: 23531] | | |
| ENSG00000203900 | AL121827.1 | Clone-based | | −0.907580244 | 0.056158339 |
| | | (Ensembl) gene | | | |
| ENSG00000204055 | AL158151.1 | Clone-based | | −0.30965968 | 0.531561049 |
| | | (Ensembl) gene | | | |
| ENSG00000204241 | AP000911.1 | Clone-based | | −0.524122921 | 0.047699169 |
| | | (Ensembl) gene | | | |
| ENSG00000204283 | LINC01973 | HGNC Symbol | long intergenic non-protein | −0.688310921 | 0.054758157 |
| | | | coding RNA 1973 | | |
| | | | [Source: HGNC Symbol; | | |
| | | | Acc: HGNC: 52800] | | |
| ENSG00000204335 | SP5 | HGNC Symbol | Sp5 transcription factor | −0.781301125 | 0.179308229 |
| | | | [Source: HGNC | | |
| | | | Symbol; Acc: HGNC: 14529] | | |
| ENSG00000204624 | DISP3 | HGNC Symbol | dispatched RND transporter | −0.556015174 | 0.112163059 |
| | | | family member 3 | | |
| | | | [Source: HGNC Symbol; | | |
| | | | Acc: HGNC: 29251] | | |
| ENSG00000204661 | C5orf60 | HGNC Symbol | chromosome 5 open reading frame 60 | −0.728340381 | 0.247590568 |
| | | | [Source: HGNC Symbol; | | |
| | | | Acc: HGNC: 27753] | | |
| ENSG00000204929 | AC007389.1 | Clone-based | | −0.641685442 | 0.282661756 |
| | | (Ensembl) gene | | | |
| ENSG00000204941 | PSG5 | HGNC Symbol | pregnancy specific | −0.381745193 | 0.571961572 |
| | | | beta-1-glycoprotein 5 | | |
| | | | [Source: HGNC Symbol; | | |
| | | | Acc: HGNC: 9522] | | |
| ENSG00000205054 | LINC01121 | HGNC Symbol | long intergenic non-protein | −0.287245288 | 0.691210517 |
| | | | coding RNA 1121 | | |
| | | | [Source: HGNC Symbol; | | |
| | | | Acc: HGNC: 49266] | | |
| ENSG00000205176 | REXD1L1P | HGNC Symbol | “REXD1 like 1, pseudogene | −0.633446822 | 0.361284725 |
| | | | [Source: HGNC | | |
| | | | Symbol; Acc: HGNC: 24660]” | | |
| ENSG00000205312 | KRT17P4 | HGNC Symbol | keratin 17 pseudogene | −0.714632356 | 0.27010304 |
| | | | 4 [Source: HGNC | | |
| | | | Symbol; Acc: HGNC: 50722] | | |
| ENSG00000205396 | LINC00661 | HGNC Symbol | long intergenic non-protein | −0.690084698 | 0.181892869 |
| | | | coding RNA 661 | | |
| | | | [Source: HGNC Symbol; | | |
| | | | Acc: HGNC: 27002] | | |
| ENSG00000205592 | MUC19 | HGNC Symbol | “mucin 19, oligomeric | −0.306611845 | 0.552328817 |
| | | | [Source: HGNC | | |
| | | | Symbol; Acc: HGNC: 14362]” | | |
| ENSG00000205667 | ARSH | HGNC Symbol | arylsulfatase family | −0.537595932 | 0.388904138 |
| | | | member H [Source: HGNC | | |
| | | | Symbol; Acc: HGNC: 32488] | | |
| ENSG00000205922 | DNECUT3 | HGNC Symbol | one cut homeobox 3 [Source: HGNC | −0.673541588 | 0.251060423 |
| | | | Symbol; Acc: HGNC: 13399] | | |
| ENSG00000205976 | AC091951.1 | Clone-based | | −0.72127846 | 0.22466354 |
| | | (Ensembl) gene | | | |
| ENSG00000210127 | MT-TA | HGNC Symbol | mitochoodrially encoded | −0.730149542 | 0.078668046 |
| | | | tRNA alanine | | |
| | | | [Source: HGNC Symbol; | | |
| | | | Acc: HGNC: 7475] | | |
| ENSG00000213467 | HMGB1P37 | HGNC Symbol | high mobility group box | −1.143333307 | 0.002787608 |
| | | | 1 pseudogene 37 | | |
| | | | [Source: HGNC Symbol; | | |
| | | | Acc: HGNC: 39184] | | |
| ENSG00000213864 | EEF1B2P2 | HGNC Symbol | eukaryotic translation elongation | −1.164370093 | 0.00450201 |
| | | | factor 1 beta 2 | | |
| | | | pseudogene 2 [Source: HGNC | | |
| | | | Symbol; Acc: HGNC: 3209] | | |
| ENSG00000213934 | HBG1 | HGNC Symbol | hemoglobin subunit | −1.506132934 | 0.001753409 |
| | | | gamma 1 [Source: HGNC | | |
| | | | Symbol; Acc: HGNC: 4831] | | |
| ENSG00000214128 | TMEM213 | HGNC Symbol | transmembrane protein | −0.721138795 | 0.190125378 |
| | | | 213 [Source: HGNC | | |
| | | | Symbol; Acc: HGNC: 27220] | | |
| ENSG00000214181 | NA | NA | NA | −0.970789027 | 0.013222867 |
| ENSG00000214402 | LCNL1 | HGNC Symbol | lipocalin like 1 [Source: HGNC | −0.706465394 | 0.073719528 |
| | | | Symbol; Acc: HGNC: 34436] | | |
| ENSG00000214929 | SPATA31D1 | HGNC Symbol | SPATA31 subfamily D | −0.65113278 | 0.36620383 |
| | | | member 1 [Source: HGNC | | |
| | | | Symbol; Acc: HGNC: 37283] | | |
| ENSG00000215405 | NA | NA | NA | −0.617865475 | 0.352456865 |
| ENSG00000215864 | NBPF7 | HGNC Symbol | NBPF member 7 [Source: HGNC | −0.90843358 | 0.018653866 |
| | | | Symbol; Acc: HGNC: 31989] | | |
| ENSG00000215895 | AL354702.1 | Clone-based | | −0.782838755 | 0.075685055 |
| | | (Ensembl) gene | | | |
| ENSG00000217094 | PPIAP31 | HGNC Symbol | peptidylprolyl isomerase | −0.998868816 | 0.000348277 |
| | | | A pseudogene 31 | | |
| | | | [Source: HGNC Symbol; | | |
| | | | Acc: HGNC: 44962] | | |
| ENSG00000218586 | AC006971.1 | Clone-based | | −0.955494818 | 0.015850348 |
| | | (Ensembl) gene | | | |
| ENSG00000218672 | AC008060.1 | Clone-based | | −0.770845732 | 0.19404204 |
| | | (Ensembl) gene | | | |
| ENSG00000218823 | PAPOLB | HGNC Symbol | poly(A) polymerase | −0.719493482 | 0.27787686 |
| | | | beta [Source: HGNC | | |
| | | | Symbol; Acc: HGNC: 15970] | | |
| ENSG00000221826 | PSG3 | HGNC Symbol | pregnancy specific | −0.240003956 | 0.750637339 |
| | | | beta-1-glycoprotein 3 | | |
| | | | [Source: HGNC Symbol; | | |
| | | | Acc: HGNC: 9520] | | |
| ENSG00000221878 | PSG7 | HGNC Symbol | pregnancy specific | −0.453292662 | 0.540259029 |
| | | | beta-1-glycoprotein 7 | | |
| | | | (gene/pseudogene) [Source: HGNC | | |
| | | | Symbol; Acc: HGNC: 9524] | | |
| ENSG00000223414 | LINC00473 | HGNC Symbol | long intergenic non-protein | −1.017484945 | 0.084276877 |
| | | | coding RNA 473 | | |
| | | | [Source: HGNC Symbol; | | |
| | | | Acc: HGNC: 21160] | | |
| ENSG00000223566 | TNRC18P2 | HGNC Symbol | trinucleotide repeat | −0.653114849 | 0.205565205 |
| | | | containing 18 pseudogene 2 | | |
| | | | [Source: HGNC Symbol; | | |
| | | | Acc: HGNC: 34014] | | |
| ENSG00000223949 | ROR1-AS1 | HGNC Symbol | ROR1 antisense RNA | −0.645751328 | 0.290941527 |
| | | | 1 [Source: HGNC | | |
| | | | Symbol; Acc: HGNC: 40508] | | |
| ENSG00000224059 | HSPA8P16 | HGNC Symbol | heat shock protein family | −1.139741884 | 0.026824728 |
| | | | A (Hsp70) member 8 | | |
| | | | pseudogene 16 [Source: HGNC | | |
| | | | Symbol; Acc: HGNC: 44931] | | |
| ENSG00000224209 | LINC00466 | HGNC Symbol | long intergenic non-protein | −1.176248621 | 0.039820431 |
| | | | coding RNA 466 | | |
| | | | [Source: HGNC Symbol; | | |
| | | | Acc: HGNC: 27294] | | |
| ENSG00000224271 | AL117329.1 | Clone-based | | −0.663993539 | 0.277648202 |
| | | (Ensembl) gene | | | |
| ENSG00000224367 | OACYLP | HGNC Symbol | “O-acyltransferase | −0.608155491 | 0.296095547 |
| | | | like, pseudogene | | |
| | | | [Source: HGNC Symbol; | | |
| | | | Acc: HGNC: 44362]” | | |
| ENSG00000224435 | NF1P6 | HGNC Symbol | neurofibromin 1 pseudogene | −0.892662927 | 0.117501042 |
| | | | 6 [Source: HGNC | | |
| | | | Symbol; Acc: HGNC: 7771] | | |
| ENSG00000224668 | IPD8P1 | HGNC Symbol | importin 8 pseudogene | −0.694695462 | 0.099383688 |
| | | | 1 [Source: HGNC | | |
| | | | Symbol; Acc: HGNC: 41955] | | |
| ENSG00000224743 | TEX26-AS1 | HGNC Symbol | TEX26 anitsense RNA | −0.60820906 | 0.280832206 |
| | | | 1 [Source: HGNC | | |
| | | | Symbol; Acc: HGNC: 42784] | | |
| ENSG00000225637 | AP001046.1 | Clone-based | | −0.681940896 | 0.281192155 |
| | | (Ensembl) gene | | | |
| ENSG00000225649 | AC064875.1 | Clone-based | | −0.698790548 | 0.326326486 |
| | | (Ensembl) gene | | | |
| ENSG00000225813 | AC009299.1 | Clone-based | | −0.210665195 | 0.744296595 |
| | | (Ensembl) gene | | | |
| ENSG00000225953 | SATB2-AS1 | HGNC Symbol | SATB2 antisense RNA | −0.527452078 | 0.302412079 |
| | | | 1 [Source: HGNC | | |
| | | | Symbol; Acc: HGNC: 26490] | | |
| ENSG00000226057 | PHF2P2 | HGNC Symbol | PHD finger protein 2 pseudogene 2 | −0.337369658 | 0.356815552 |
| | | | [Source: HGNC Symbol; | | |
| | | | Acc: HGNC: 38808] | | |
| ENSG00000226068 | HNRNPA3P4 | HGNC Symbol | heterogeneous nuclear | −1.120089183 | 0.013553682 |
| | | | ribonucleoprotein A3 | | |
| | | | pseudogene 4 [Source: HGNC | | |
| | | | Symbol; Acc: HGNC: 39773] | | |
| ENSG00000226440 | AL365214.2 | Clone-based | | −0.699415602 | 0.287780065 |
| | | (Ensembl) gene | | | |
| ENSG00000226741 | LINC02554 | HGNC Symbol | long intergenic | −0.684878038 | 0.273326765 |
| | | | non-coding RNA 2554 | | |
| | | | [Source: HGNC Symbol; | | |
| | | | Acc: HGNC: 53594] | | |
| ENSG00000226790 | HNRNPA3P1 | HGNC Symbol | heterogeneous nuclear | −1.384734034 | 0.000189579 |
| | | | ribonucleoprotein A3 | | |
| | | | pseudogene 1 [Source: HGNC | | |
| | | | Symbol; Acc: HGNC: 13729] | | |
| ENSG00000227036 | LINC00511 | HGNC Symbol | long intergenic non-protein | −0.508830938 | 0.153757922 |
| | | | coding RNA 511 | | |
| | | | [Source: HGNC Symbol; | | |
| | | | Acc: HGNC: 43564] | | |
| ENSG00000227471 | AKR1B15 | HGNC Symbol | aldo-keto reductase family | −0.827071435 | 0.171395362 |
| | | | 1 member B15 | | |
| | | | [Source: HGNC Symbol; | | |
| | | | Acc: HGNC: 37281] | | |
| ENSG00000227525 | RPL7P6 | HGNC Symbol | ribosomal protein L7 pseudogene 6 | −1.209818266 | 0.003030406 |
| | | | [Source: HGNC Symbol; | | |
| | | | Acc: HGNC: 32430] | | |
| ENSG00000227744 | LINC01940 | HGNC Symbol | long intergenic non-protein | −0.606198906 | 0.304426384 |
| | | | coding RNA 1940 | | |
| | | | [Source: HGNC Symbol; | | |
| | | | Acc: HGNC: 52763] | | |
| ENSG00000227827 | AC138969.2 | Clone-based | | −0.592806597 | 0.166783813 |
| | | (Ensembl) gene | | | |
| ENSG00000228549 | BX284668.2 | Clone-based | | −0.68006559 | 0.067244282 |
| | | (Ensembl) gene | | | |
| ENSG00000228983 | SLC47A1P1 | HGNC Symbol | solute carrier family 47 | −0.809148556 | 0.2081298 |
| | | | member 1 pseudogene 1 | | |
| | | | [Source: HGNC Symbol; | | |
| | | | Acc: HGNC: 51849] | | |
| ENSG00000229147 | SMPD4P2 | HGNC Symbol | sphingomyelin phosphodiesterase | −0.546120346 | 0.368203078 |
| | | | 4 pseudogene | | |
| | | | 2 [Source: HGNC Symbol; | | |
| | | | Acc: HGNC: 39674] | | |
| ENSG00000229240 | LINC00710 | HGNC Symbol | long intergenic non-protein | −0.349696304 | 0.636769038 |
| | | | coding RNA 710 | | |
| | | | [Source: HGNC Symbol; | | |
| | | | Acc: HGNC: 27386] | | |
| ENSG00000229817 | AL133412.1 | Clone-based | | −1.134095271 | 0.031106855 |
| | | (Ensembl) gene | | | |
| ENSG00000229972 | IQCF3 | HGNC Symbol | IQ motif containing | −0.707465438 | 0.238864276 |
| | | | F3 [Source: HGNC | | |
| | | | Symbol; Acc: HGNC: 31816] | | |
| ENSG00000230102 | LINC0208 | HGNC Symbol | long intergenic non-protein | −0.84996372 | 0.156994187 |
| | | | coding RNA 2028 | | |
| | | | [Source: HGNC Symbol; | | |
| | | | Acc: HGNC: 27718] | | |
| ENSG00000230133 | LINC01721 | HGNC Symbol | long intergenic non-protein | −0.269403043 | 0.711053504 |
| | | | coding RNA 1721 | | |
| | | | [Source: HGNC Symbol; | | |
| | | | Acc: HGNC: 52508] | | |
| ENSG00000230392 | AC004835.1 | Clone-based | | −0.84709631 | 0.102864131 |
| | | (Ensembl) gene | | | |
| ENSG00000230552 | AC092162.2 | Clone-based | | −0.369157666 | 0.571101565 |
| | | (Ensembl) gene | | | |
| ENSG00000230615 | AL139220.2 | Clone-based | | −0.52204036 | 0.382207592 |
| | | (Ensembl) gene | | | |
| ENSG00000230873 | STMND1 | HGNC Symbol | stathmin domain containing | −0.360296128 | 0.61243615 |
| | | | 1 [Source: HGNC | | |
| | | | Symbol; Acc: HGNC: 44668] | | |
| ENSG00000231131 | LINC01468 | HGNC Symbol | long intergenic non-protein | −0.539334563 | 0.42259164 |
| | | | coding RNA 1468 | | |
| | | | [Source: HGNC Symbol; | | |
| | | | Acc: HGNC: 50913] | | |
| ENSG00000231422 | LINC01516 | HGNC Symbol | long intergenic non-protein | −0.556463466 | 0.39546555 |
| | | | coding RNA 1516 | | |
| | | | [Source: HGNC Symbol; | | |
| | | | Acc: HGNC: 51211] | | |
| ENSG00000232392 | AC002366.1 | Clone-based | | −0.504607303 | 0.313754361 |
| | | (Ensembl) gene | | | |
| ENSG00000232667 | AC004862.1 | Clone-based | | −0.377421732 | 0.617985726 |
| | | (Ensembl) gene | | | |
| ENSG00000232756 | AC004996.1 | Clone-based | | −0.532711499 | 0.428875064 |
| | | (Ensembl) gene | | | |
| ENSG00000233183 | AL138889.1 | Clone-based | | −0.644340746 | 0.293569058 |
| | | (Ensembl) gene | | | |
| ENSG00000233395 | LINC00841 | HGNC Symbol | long intergenic non-protein | −0.755094485 | 0.24458117 |
| | | | coding RNA 841 | | |
| | | | [Source: HGNC Symbol; | | |
| | | | Acc: HGNC: 27430] | | |
| ENSG00000233485 | AL031283.2 | Clone-based | | −0.639007269 | 0.295175271 |
| | | (Ensembl) gene | | | |
| ENSG00000233539 | AC011294.1 | Clone-based | | −0.707276432 | 0.262329375 |
| | | (Ensembl) gene | | | |
| ENSG00000233973 | LINC01360 | HGNC Symbol | long intergenic non-protein | −0.427339916 | 0.546690871 |
| | | | coding RNA 1360 | | |
| | | | [Source: HGNC Symbol; | | |
| | | | Acc: HGNC: 50593] | | |
| ENSG00000233977 | AC099681.3 | Clone-based | | −1.209272617 | 0.027526442 |
| | | (Ensembl) gene | | | |
| ENSG00000233991 | NA | NA | NA | −0.525516632 | 0.310266093 |
| ENSG00000234130 | AL359263.1 | Clone-based | | −0.983040041 | 0.004471167 |
| | | (Ensembl) gene | | | |
| ENSG00000234177 | LINC01114 | HGNC Symbol | long intergenic non-protein | −0.49846577 | 0.434856109 |
| | | | coding RNA 1114 | | |
| | | | [Source: HGNC Symbol; | | |
| | | | Acc: HGNC: 49245] | | |
| ENSG00000234512 | TLR12P | HGNC Symbol | “toll like receptor 12, pseudogene | −0.514872503 | 0.441103354 |
| | | | [Source: HGNC Symbol; | | |
| | | | Acc: HGNC: 31754]” | | |
| ENSG00000234537 | AL354751.1 | Clone-based | | −0.384649992 | 0.532855004 |
| | | (Ensembl) gene | | | |
| ENSG00000234828 | IQCM | HGNC Symbol | IQ motif containing | −0.677274946 | 0.33050019 |
| | | | M [Source: HGNC | | |
| | | | Symbol; Acc: HGNC: 53443] | | |
| ENSG00000235711 | ANKRD34C | HGNC Symbol | ankyrin repeat domain | −0.629664373 | 0.321694729 |
| | | | 34C [Source: HGNC | | |
| | | | Symbol; Acc: HGNC: 33888] | | |
| ENSG00000235881 | AC114776.1 | Clone-based | | −0.567508401 | 0.40045344 |
| | | (Ensembl) gene | | | |
| ENSG00000236078 | LINC01447 | HGNC Symbol | long intergenic non-protein | −0.901815564 | 0.106966165 |
| | | | ending RNA 1447 | | |
| | | | [Source: HGNC Symbol; | | |
| | | | Acc: HGNC: 50783] | | |
| ENSG00000236229 | VEZF1P1 | HGNC Symbol | vascular endothelial zinc | −0.741631818 | 0.120281894 |
| | | | finger 1 pseudogene 1 | | |
| | | | [Source: HGNC Symbol; | | |
| | | | Acc: HGNC: 32320] | | |
| ENSG00000236253 | SLC25A3P1 | HGNC Symbol | solute carrier family | −0.817459523 | 0.156635614 |
| | | | 25 member 3 pseudogene 1 | | |
| | | | [Source: HGNC Symbol; | | |
| | | | Acc: HGNC: 26869] | | |
| ENSG00000236404 | VLDLR-AS1 | HGNC Symbol | VLDLR antisense RNA | −0.617831635 | 0.280386176 |
| | | | 1 [Source: HGNC | | |
| | | | Symbol; Acc: HGNC: 49621] | | |
| ENSG00000236445 | LINC00608 | HGNC Symbol | long intergenic non-protein | −0.743359785 | 0.28126695 |
| | | | coding RNA 608 | | |
| | | | [Source: HGNC Symbol; | | |
| | | | Acc: HGNC: 27179] | | |
| ENSG00000236824 | BCYRN1 | HGNC Symbol | brain cytoplasmic | −0.656526317 | 0.083153215 |
| | | | RNA 1 [Source: HGNC | | |
| | | | Symbol; Acc: HGNC: 1022] | | |
| ENSG00000237125 | HAND2-AS1 | HGNC Symbol | HAND2 antisense RNA 1 | −0.621343361 | 0.260507627 |
| | | | (head to head) | | |
| | | | [Source: HGNC Symbol; | | |
| | | | Acc: HGNC: 48872] | | |
| ENSG00000237222 | LINC01968 | HGNC Symbol | long intergenic non-protein | −0.672454358 | 0.277770947 |
| | | | coding RNA 1968 | | |
| | | | [Source: HGNC Symbol; | | |
| | | | Acc: HGNC: 52794] | | |
| ENSG00000237250 | AL359924.1 | Clone-based | | −0.844197088 | 0.255602335 |
| | | (Ensembl) gene | | | |
| ENSG00000237289 | CKMT1B | HGNC Symbol | “creatine kinase, | −0.216490618 | 0.795077893 |
| | | | mitochondrial 1B | | |
| | | | [Source: HGNC Symbol; | | |
| | | | Acc: HGNC: 1995]” | | |
| ENSG00000237390 | AL139130.1 | Clone-based | | −0.681458963 | 0.223257294 |
| | | (Ensembl) gene | | | |
| ENSG00000237515 | SHISA9 | HGNC Symbol | shisa family member | −0.809010055 | 0.106410732 |
| | | | 9 [Source: HGNC | | |
| | | | Symbol; Acc: HGNC: 37231] | | |
| ENSG00000237636 | ANKRD26P3 | HGNC Symbol | ankyrin repeat domain | −0.387705186 | 0.561900253 |
| | | | 26 pseudogene 3 | | |
| | | | [Source: HGNC Symbol; | | |
| | | | Acc: HGNC: 39689] | | |
| ENSG00000238116 | Z95327.1 | Clone-based | | −1.006307186 | 0.058635061 |
| | | (Ensembl) gene | | | |
| ENSG00000239921 | LINC01471 | HGNC Symbol | long intergenic non-protein | −0.433460557 | 0.515402598 |
| | | | coding RNA 1471 | | |
| | | | [Source: HGNC Symbol; | | |
| | | | Acc: HGNC: 51106] | | |
| ENSG00000240021 | TEX35 | HGNC Symbol | testis expressed | −0.374999293 | 0.569064774 |
| | | | 35 [Source: HGNC | | |
| | | | Symbol; Acc: HGNC: 25366] | | |
| ENSG00000240694 | PNMA2 | HGNC Symbol | PNMA family member | −0.937890632 | 0.011844862 |
| | | | 2 [Source: HGNC | | |
| | | | Symbol; Acc: HGNC: 9159] | | |
| ENSG00000240707 | LINC01168 | HGNC Symbol | long intergenic non-protein | −0.843835897 | 0.086801762 |
| | | | coding RNA 1168 | | |
| | | | [Source: HGNC Symbol; | | |
| | | | Acc: HGNC: 49537] | | |
| ENSG00000241158 | ADAMTS9-AS1 | HGNC Symbol | ADAMTS9 antisense | −0.408694485 | 0.583233437 |
| | | | RNA 1 [Source: HGNC | | |
| | | | Symbol; Acc: HGNC: 40625] | | |
| ENSG00000242512 | LINC01206 | HGNC Symbol | long intergenic non-protein | −0.706615823 | 0.298614872 |
| | | | coding RNA 1206 | | |
| | | | [Source: HGNC Symbol; | | |
| | | | Acc: HGNC: 49637] | | |
| ENSG00000242866 | STRC | HGNC Symbol | stereocilin [Source: HGNC | −0.378462607 | 0.423980174 |
| | | | Symbol; Acc: HGNC: 16035] | | |
| ENSG00000243130 | PSG11 | HGNC Symbol | pregnancy specific | −0.64081905 | 0.353755564 |
| | | | beta-1-glycoprotein 11 | | |
| | | | [Source: HGNC Symbol; | | |
| | | | Acc: HGNC: 9516] | | |
| ENSG00000244694 | PTCHD4 | HGNC Symbol | patched domain containing | −0.961630256 | 0.109326576 |
| | | | 4 [Source: HGNC | | |
| | | | Symbol; Acc: HGNC: 21345] | | |
| ENSG00000245248 | USP2-AS1 | HGNC Symbol | DSP2 antisense RNA 1 (head to head) | −0.520768111 | 0.390756877 |
| | | | [Source: HGNC Symbol; | | |
| | | | Acc: HGNC: 48673] | | |
| ENSG00000245651 | AC083805.1 | Clone-based | | −0.541469632 | 0.389457898 |
| | | (Ensembl) gene | | | |
| ENSG00000246695 | RASSF8-AS1 | HGNC Symbol | RASSF8 antisense RNA | −0.616821021 | 0.302209736 |
| | | | 1 [Source: HGNC | | |
| | | | Symbol; Acc: HGNC: 48637] | | |
| ENSG00000247213 | LINC01498 | HGNC Symbol | long intergenic non-protein | −0.955409024 | 0.107829748 |
| | | | coding RNA 1498 | | |
| | | | [Source: HGNC Symbol; | | |
| | | | Acc: HGNC: 51164] | | |
| ENSG00000247699 | AC008609.1 | Clone-based | | −1.050990069 | 0.073837393 |
| | | (Ensembl) gene | | | |
| ENSG00000248587 | GDNF-AS1 | HGNC Symbol | GDNF antisense RNA 1 | −0.698157573 | 0.21408791 |
| | | | (head to head) | | |
| | | | [Source: HGNC Symbol; | | |
| | | | Acc: HGNC: 43592] | | |
| ENSG00000248713 | AC083902.2 | Clone-based | “Homo sapiens | −0.540713726 | 0.305645261 |
| | | (Ensembl) gene | uncharacterized LOC285556 | | |
| | | | (LOC285556), mRNA. [Source: RefSeq | | |
| | | | mRNA; Acc: NM_001354435]” | | |
| ENSG00000248746 | ACTN3 | HGNC Symbol | actinin alpha 3 (gene/pseudogene) | −0.833558399 | 0.085185005 |
| | | | [Source: HGNC Symbol; | | |
| | | | Acc: HGNC: 165] | | |
| ENSG00000248966 | BCLAF1P1 | HGNC Symbol | BCL2 associated transcription factor 1 | −0.901954794 | 0.005426121 |
| | | | pseudogene 1 [Source: HGNC | | |
| | | | Symbol; Acc: HGNC: 51329] | | |
| ENSG00000248975 | AL133372.2 | Clone-based | | −0.655164585 | 0.316622442 |
| | | (Ensembl) gene | | | |
| ENSG00000249119 | MTNDGP4 | HGNC Symbol | mitochondrially encoded | −1.255534276 | 0.007501713 |
| | | | NADH: ubiquinone | | |
| | | | oxidoreductase care | | |
| | | | subunit 6 pseudogene 4 | | |
| | | | [Source: HGNC Symbol; | | |
| | | | Acc: HGNC: 39467] | | |
| ENSG00000249215 | NCOA4P4 | HGNC Symbol | nuclear receptor coactivator | −1.297109171 | 0.000137132 |
| | | | 4 pseudogene 4 | | |
| | | | [Source: HGNC Symbol; | | |
| | | | Acc: HGNC: 52405] | | |
| ENSG00000249267 | LINC00939 | HGNC Symbol | long intergenic non-protein | −0.755105061 | 0.228670526 |
| | | | coding RNA 939 | | |
| | | | [Source: HGNC Symbol; | | |
| | | | Acc: HGNC: 48631] | | |
| ENSG00000249341 | AC124017.1 | Clone-based | | −0.838158018 | 0.180191435 |
| | | (Ensembl) gene | | | |
| ENSG00000249464 | LINC01091 | HGNC Symbol | long intergenic non-protein | −0.927878847 | 0.088700609 |
| | | | coding RNA 1091 | | |
| | | | [Source: HGNC Symbol; | | |
| | | | Acc: HGNC: 27721] | | |
| ENSG00000249584 | LINC02225 | HGNC Symbol | long intergenic non-protein | −0.580968221 | 0.346216369 |
| | | | coding RNA 2225 | | |
| | | | [Source: HGNC Symbol; | | |
| | | | Acc: HGNE: 53094] | | |
| ENSG00000249618 | LINC02465 | HGNC Symbol | long intergenic non-protein | −0.994691738 | 0.119254417 |
| | | | coding RNA 2465 | | |
| | | | [Source: HGNC Symbol; | | |
| | | | Acc: HGNC: 53403] | | |
| ENSG00000250049 | AC114316.2 | Clone-based | | −0.734378499 | 0.217922591 |
| | | (Ensembl) gene | | | |
| ENSG00000250230 | AP002754.1 | Clone-based | | −1.010448463 | 0.076383023 |
| | | (Ensembl) gene | | | |
| ENSG00000250420 | AACSP1 | HGNC Symbol | acetoacetyl-CoA synthetase | −0.85134176 | 0.138526408 |
| | | | pseudogene 1 | | |
| | | | [Source: HGNC Symbol; | | |
| | | | Acc: HGNC: 18226] | | |
| ENSG00000250423 | KIAA1210 | HGNC Symbol | KIAA1210 [Source: HGNC Symbol; | −0.616623881 | 0.302589969 |
| | | | Acc: HGNC: 29218] | | |
| ENSG00000250493 | AP004147.1 | Clone-based | | −0.423508937 | 0.487573965 |
| | | (Ensembl) gene | | | |
| ENSG00000250519 | AP002784.1 | Clone-based | | −0.661885269 | 0.300298544 |
| | | (Ensembl) gene | | | |
| ENSG00000250546 | AC079160.1 | Clone-based | | −0.594876334 | 0.407745799 |
| | | (Ensembl) gene | | | |
| ENSG00000251557 | HNRNPKP3 | HGNC Symbol | heterogenous nuclear | −0.983325003 | 0.155739901 |
| | | | ribonucleoprotein K | | |
| | | | pseudogene 3 [Source: HGNC | | |
| | | | Symbol; Acc: HGNC: 42376] | | |
| ENSG00000251596 | HADHAP1 | HGNC Symbol | “hydroxyacyl-CoA | −0.74139099 | 0.215412986 |
| | | | dehydrogenase/3-ketoacyl-CoA | | |
| | | | thiolase/enoyl-CoA hydratase | | |
| | | | (trifunctional | | |
| | | | protein), alpha subunit pseudogene 1 | | |
| | | | [Source: HGNC Symbol; | | |
| | | | Acc: HGNC: 4802]” | | |
| ENSG00000251705 | RNA5-8SP6 | HGNC Symbol | “RNA, 5.8S ribosomal pseudogene 6 | −0.425991485 | 0.294278305 |
| | | | [Source: HGNC Symbol; | | |
| | | | Acc: HGNC: 41960]” | | |
| ENSG00000253230 | LINC00599 | HGNC Symbol | long intergenic non-protein | −0.760456332 | 0.135076382 |
| | | | coding RNA 599 | | |
| | | | [Source: HGNC Symbol; | | |
| | | | Acc: HGNC: 27231] | | |
| ENSG00000253288 | AC046195.1 | Clone-based | | −0.709937286 | 0.305188446 |
| | | (Ensembl) gene | | | |
| ENSG00000253301 | LINC01606 | HGNC Symbol | long intergenic non-protein | −0.711369904 | 0.234542306 |
| | | | coding RNA 1606 | | |
| | | | [Source: HGNC Symbol; | | |
| | | | Acc: HGNC: 51656] | | |
| ENSG00000253864 | NA | NA | NA | −0.889113296 | 0.11585236 |
| ENSG00000253871 | AC068075.1 | Clone-based | | −1.11237045 | 0.076114677 |
| | | (Ensembl) gene | | | |
| ENSG00000253974 | NRG1-IT1 | HGNC Symbol | NRG1 intrunic transcript | −0.501373766 | 0.471813581 |
| | | | 1 [Source: HGNC | | |
| | | | Symbol; Acc: HGNC: 43633] | | |
| ENSG00000254042 | ACD11365.1 | Clone-based | | −0.600224259 | 0.364960528 |
| | | (Ensembl) gene | | | |
| ENSG00000254101 | LINC02055 | HGNC Symbol | long intergenic non-protein | −0.903549507 | 0.185220622 |
| | | | coding RNA 2055 | | |
| | | | [Source: HGNC Symbol; | | |
| | | | Acc: HGNC: 52895] | | |
| ENSG00000254561 | AP003393.1 | Clone-based | | −0.734861559 | 0.197377124 |
| | | (Ensembl) gene | | | |
| ENSG00000256343 | AC095350.1 | Clone-based | | −0.895023552 | 0.169037894 |
| | | (Ensembl) gene | | | |
| ENSG00000256612 | CYP2B7P | HGNC Symbol | “cytochrome P450 family | −0.489134845 | 0.20391081 |
| | | | 2 subfamily B member | | |
| | | | 7, pseudogene [Source: HGNC | | |
| | | | Symbol; Acc: HGNC: 2616]” | | |
| ENSG00000256616 | AP002414.2 | Clone-based | | −0.533801034 | 0.439483446 |
| | | (Ensembl) gene | | | |
| ENSG00000257008 | GPR142 | HGNC Symbol | G protein-coupled receptor | −0.88549094 | 0.103049732 |
| | | | 142 [Source: HGNC | | |
| | | | Symbol; Acc: HGNC: 20088] | | |
| ENSG00000257175 | CR383656.1 | Clone-based | | −0.641868034 | 0.142758994 |
| | | (Ensembl) gene | | | |
| ENSG00000257576 | HSPD1P4 | HGNC Symbol | heat shock prat Bin | −0.870496882 | 0.027959144 |
| | | | family D (Hsp60) member 1 | | |
| | | | pseudogene 4 [Source: HGNC | | |
| | | | Symbol; Acc: HGNC: 35146] | | |
| ENSG00000257907 | EEF1A1P17 | HGNC Symbol | eukaryotic translation | −1.061336051 | 0.012387251 |
| | | | elongation factor 1 alpha 1 | | |
| | | | pseudogene 17 [Source: HGNC | | |
| | | | Symbol; Acc: HGNC: 37890] | | |
| ENSG00000258628 | AC126603.1 | Clone-based | | −0.496381087 | 0.388904138 |
| | | (Ensembl) gene | | | |
| ENSG00000258679 | AC121758.1 | Clone-based | | −0.657256137 | 0.268271188 |
| | | (Ensembl) gene | | | |
| ENSG00000259010 | AL049869.1 | Clone-based | | −1.06237153 | 0.004353922 |
| | | (Ensembl) gene | | | |
| ENSG00000259156 | CHEK2P2 | HGNC Symbol | checkpoint kinase 2 pseudogene | −0.816062698 | 0.100260967 |
| | | | 2 [Source: HGNC | | |
| | | | Symbol; Acc: HGNC: 43578] | | |
| ENSG00000259176 | NA | NA | NA | −0.689122052 | 0.24300017 |
| ENSG00000259380 | AC087473.1 | Clone-based | | −0.259518718 | 0.707119428 |
| | | (Ensembl) gene | | | |
| ENSG00000259458 | AC100839.1 | Clone-based | | −0.711262016 | 0.226889647 |
| | | (Ensembl) gene | | | |
| ENSG00000259790 | ANP32BP1 | HGNC Symbol | acidic nuclear phosphoprotein | −0.99159785 | 0.001291068 |
| | | | 32 family member | | |
| | | | B pseudogene 1 [Source: HGNC | | |
| | | | Symbol; Acc: HGNC: 24267] | | |
| ENSG00000259841 | LINC01566 | HGNC Symbol | long intergenic non-protein | −0.459757156 | 0.519499356 |
| | | | coding RNA 1566 | | |
| | | | [Source: HGNC Symbol; | | |
| | | | Acc: HGNC: 27555] | | |
| ENSG00000260027 | HOXB7 | HGNC Symbol | homeobox B7 [Source: HGNC | −0.542434326 | 0.225372277 |
| | | | Symbol; Acc: HGNC: 5118] | | |
| ENSG00000260072 | AC008938.1 | Clone-based | | −0.616696682 | 0.313465888 |
| | | (Ensembl) gene | | | |
| ENSG00000260290 | AC092115.1 | Clone-based | | −0.846632607 | 0.001798958 |
| | | (Ensembl) gene | | | |
| ENSG00000260305 | NTRK3-AS1 | HGNC Symbol | NTRK3 antisense RNA | −0.409045013 | 0.529909289 |
| | | | 1 [Source: HGNC | | |
| | | | Symbol; Acc: HGNC: 27532] | | |
| ENSG00000260411 | NA | NA | NA | −0.513027268 | 0.393639567 |
| ENSG00000260759 | AP001120.1 | Clone-based | | −0.782720245 | 0.215023019 |
| | | (Ensembl) gene | | | |
| ENSG00000261104 | AC093904.4 | Clone-based | | −0.334778367 | 0.600661825 |
| | | (Ensembl) gene | | | |
| ENSG00000261115 | TMEM178B | HGNC Symbol | transmembrane protein | −0.352100285 | 0.265283771 |
| | | | 178B [Source: HGNC | | |
| | | | Symbol; Acc: HGNC: 44112] | | |
| ENSG00000261177 | AC135012.1 | Clone-based | | −0.994275816 | 0.048314645 |
| | | (Ensembl) gene | | | |
| ENSG00000261275 | AC092447.8 | Clone-based | | −0.812472764 | 0.203816116 |
| | | (Ensembl) gene | | | |
| ENSG00000261466 | AC136944.4 | Clone-based | | −1.109539774 | 0.041757833 |
| | | (Ensembl) gene | | | |
| ENSG00000261623 | LINC02179 | HGNC Symbol | long intergenic non-protein | −0.11447084 | 0.908308893 |
| | | | coding RNA 2179 | | |
| | | | [Source: Symbol; | | |
| | | | Acc: HGNC: 53041] | | |
| ENSG00000261649 | GOLGA6L7 | HGNC Symbol | golgin A6 family like | −1.108323245 | 0.018730492 |
| | | | 7 [Source: HGNC | | |
| | | | Symbol; Acc: HGNC: 37442] | | |
| ENSG00000261949 | GFY | HGNC Symbol | golgi associated olfactory | −0.815057864 | 0.178504736 |
| | | | signaling regulator | | |
| | | | [Source: HGNC Symbol; | | |
| | | | Acc: HGNC: 44663] | | |
| ENSG00000262691 | AC040160.1 | Clone-based | | −0.536786924 | 0.387929026 |
| | | (Ensembl) gene | | | |
| ENSG00000263711 | AC079062.1 | Clone-based | | −0.664230562 | 0.292210006 |
| | | (Ensembl) gene | | | |
| ENSG00000264954 | PRR29-AS1 | HGNC Symbol | PRR29 antisense RNA1 | −0.735900318 | 0.102864131 |
| | | | [Source: HGNC | | |
| | | | Symbol; Acc: HGNC: 51822] | | |
| ENSG00000265041 | AC015688.3 | Clone-based | | −0.765364276 | 0.178504736 |
| | | (Ensembl) gene | | | |
| ENSG00000266172 | NA | NA | NA | −0.643514714 | 0.277770947 |
| ENSG00000266795 | NA | NA | NA | −0.903848134 | 0.094040442 |
| ENSG00000267324 | AC006557.2 | Clone-based | | −0.380979397 | 0.536886334 |
| | | (Ensembl) gene | | | |
| ENSG00000268388 | FENDRR | HGNC Symbol | FDXF1 adjacent non-coding | −0.688358604 | 0.302352803 |
| | | | developmental | | |
| | | | regulatory RNA [Source: HGNC | | |
| | | | Symbol; Acc: HGNC: 43894] | | |
| ENSG00000268460 | AC006262.1 | Clone-based | | −0.863478705 | 0.141982169 |
| | | (Ensembl) gene | | | |
| ENSG00000269332 | GOLGA2P9 | HGNC Symbol | golgin A2 pseudogene | −0.795855321 | 0.179308229 |
| | | | 9 (Source: HGNC | | |
| | | | Symbol; Acc: HGNC: 49921] | | |

| ensembl | p16.logFC | p16.FDR | p17.logFC | p17.FDR | p18.logFC | p18.FDR |
|---|---|---|---|---|---|---|
| ENSG00000002746 | −0.328868097 | 1 | 0.438047831 | 0.908518526 | 0.257699857 | 1 |
| ENSG00000004948 | 0.278685382 | 1 | 2.655446254 | 0.699814612 | 0.328026413 | 1 |
| ENSG00000006071 | −0.183143093 | 1 | −0.151122062 | 0.972840456 | 0.060166206 | 1 |
| ENSG00000006128 | −0.42309878 | 1 | −0.821588596 | 0.86142478 | 0.611538787 | 1 |
| ENSG00000006210 | 0.394994483 | 1 | 0.142838907 | 0.984792321 | −0.105867018 | 1 |
| ENSG00000006283 | −0.153595375 | 1 | 0.736365852 | 0.850152372 | 0.013389601 | 1 |
| ENSG00000006788 | −0.132296216 | 1 | 1.277263446 | 0.740349802 | 0.194022822 | 1 |
| ENSG00000009709 | −0.237365593 | 1 | 6.295166726 | 0.627668963 | 0.231942676 | 1 |
| ENSG00000011677 | −0.442276362 | 1 | 1.889252428 | 0.751835169 | 0.151297142 | 1 |
| ENSG00000015520 | −0.700785246 | 1 | −0.349775321 | 0.929542543 | 0.193503425 | 1 |
| ENSG00000018607 | −0.226758688 | 1 | 0.349569946 | 0.959459365 | 0.583710154 | 1 |
| ENSG00000018625 | −0.323676225 | 1 | 1.616286887 | 0.721222226 | 0.112816854 | 1 |
| ENSG00000019505 | 0.192851682 | 1 | 1.766660549 | 0.735101069 | −0.059220871 | 1 |
| ENSG00000039139 | −0.350782091 | 1 | 0.33637337 | 0.929444622 | 0.374918338 | 1 |
| ENSG00000039987 | −0.266358455 | 1 | −0.702991923 | 0.879866057 | −0.349787212 | 1 |
| ENSG00000054356 | 0.117493297 | 1 | −0.428199871 | 0.839974197 | −0.036044109 | 1 |
| ENSG00000060656 | −0.204609501 | 1 | 0.38533287 | 0.883612252 | 0.052221341 | 1 |
| ENSG00000060709 | −0.14886622 | 1 | 0.267280913 | 0.953466793 | −0.023574473 | 1 |
| ENSG00000068078 | 0.779778588 | 1 | 0.665950568 | 0.860481624 | −0.338607178 | 1 |
| ENSG00000069431 | −0.698467209 | 1 | 1.501464862 | 0.754933936 | 0.427108144 | 1 |
| ENSG00000070886 | 0.037142847 | 1 | 0.618792016 | 0.876857452 | −0.131880882 | 1 |
| ENSG00000072041 | −0.029174812 | 1 | 0.598327652 | 0.901939163 | 0.218044555 | 1 |
| ENSG00000075891 | −0.139680455 | 1 | 0.666537779 | 0.859477376 | 0.023400093 | 1 |
| ENSG00000077080 | −0.291947443 | 1 | 2.05248954 | 0.734558921 | −0.579843889 | 1 |
| ENSG00000077522 | −0.359543665 | 1 | 1.065057551 | 0.816563802 | 0.056205006 | 1 |
| ENSG00000078295 | −0.153032167 | 1 | 0.090300816 | 0.991823026 | −0.190263243 | 1 |
| ENSG00000078549 | −0.548781569 | 1 | 0.436615106 | 0.919671237 | 0.282001957 | 1 |
| ENSG00000078898 | −0.186330302 | 1 | 0.607904762 | 0.874416638 | 0.447656564 | 1 |
| ENSG00000079112 | −0.174510118 | 1 | 1.290358643 | 0.794377353 | −0.241687928 | 1 |
| ENSG00000079841 | −0.220370451 | 1 | 0.260822706 | 0.957654277 | 0.360367388 | 1 |
| ENSG00000081248 | −0.185575362 | 1 | −0.292283267 | 0.939971429 | 0.087334946 | 1 |
| ENSG00000081800 | −0.315731989 | 1 | 0.415494856 | 0.928156195 | −0.122942855 | 1 |
| ENSG00000082175 | −0.172803447 | 1 | 1.977891718 | 0.699433544 | −0.170595574 | 1 |
| ENSG00000084636 | −0.166089061 | 1 | 0.586078349 | 0.853985444 | −0.026623098 | 1 |
| ENSG00000088340 | −0.078004571 | 1 | 0.349186341 | 0.910714926 | −0.239607349 | 1 |
| ENSG00000089116 | 0.065319066 | 1 | 0.185915909 | 0.972840456 | 0.479688913 | 1 |
| ENSG00000089199 | −0.320202315 | 1 | 0.377523248 | 0.925166462 | −0.124040006 | 1 |
| ENSG00000089225 | −0.186556677 | 1 | 0.596701642 | 0.898551048 | 0.187810501 | 1 |
| ENSG00000091128 | 0.074990997 | 1 | 1.134840911 | 0.766090106 | 0.591653405 | 1 |
| ENSG00000091137 | −0.379948372 | 1 | 0.990697508 | 0.776841279 | 0.184013228 | 1 |
| ENSG00000091656 | 0.068997085 | 1 | 0.573719306 | 0.878466837 | −0.078950263 | 1 |
| ENSG00000095587 | −0.263611606 | 1 | 1.33832395 | 0.748037903 | 0.061668368 | 1 |
| ENSG00000095637 | −0.086812706 | 1 | 0.072112715 | 0.980464761 | 0.233872426 | 1 |
| ENSG00000099625 | 0.924708632 | 1 | 4.510600591 | 0.630714789 | −0.556807911 | 1 |
| ENSG00000100033 | 0.392602047 | 1 | 0.416466908 | 0.927256113 | 0.024209613 | 1 |
| ENSG00000100065 | −0.094316814 | 1 | 1.184451382 | 0.737781088 | 0.010459367 | 1 |
| ENSG00000100078 | 0.366462178 | 1 | −0.809130599 | 0.880905808 | 0.273159772 | 1 |
| ENSG00000100312 | −0.436425953 | 1 | −0.816265078 | 0.843731985 | 0.905100759 | 1 |
| ENSG00000101004 | −0.139339033 | 1 | 0.111085893 | 0.958051922 | 0.007374673 | 1 |
| ENSG00000101057 | 0.065592927 | 1 | −0.316760009 | 0.853985444 | −0.363993202 | 1 |
| ENSG00000101203 | 0.172380322 | 1 | 1.370012438 | 0.716500857 | 0.256210081 | 1 |
| ENSG00000101276 | 0.082884294 | 1 | 1.909327866 | 0.760815797 | −0.182694116 | 1 |
| ENSG00000101680 | −0.203871279 | 1 | 0.400062263 | 0.922861341 | 0.571294866 | 1 |
| ENSG00000101871 | 0.209637567 | 1 | −0.268771891 | 0.95125433 | −0.036170874 | 1 |
| ENSG00000102287 | −0.178338056 | 1 | 0.533972134 | 0.907527268 | 0.37680991 | 1 |
| ENSG00000102290 | −0.633443604 | 1 | 0.867626644 | 0.84830486 | 0.353161708 | 1 |
| ENSG00000102452 | −0.228025142 | 1 | 0.395224547 | 0.921775245 | 0.236838494 | 1 |
| ENSG00000103647 | 0.479287415 | 1 | 0.151419617 | 0.954367943 | 0.394859842 | 1 |
| ENSG00000103855 | −0.043746659 | 1 | 2.891620875 | 0.655801493 | 0.339884759 | 1 |
| ENSG00000104055 | 0.346818998 | 1 | 2.65751116 | 0.661458936 | 0.486591696 | 1 |
| ENSG00000104313 | −0.089328093 | 1 | 0.904381457 | 0.824640952 | −0.145400561 | 1 |
| ENSG00000104435 | −0.070221779 | 1 | 2.138741005 | 0.743863355 | 0.679714458 | 1 |
| ENSG00000104537 | −0.197734314 | 1 | 0.962189734 | 0.845260843 | 0.15906724 | 1 |
| ENSG00000104967 | −0.408162724 | 1 | −0.241866658 | 0.967431863 | −0.288237016 | 1 |
| ENSG00000105290 | 0.239914511 | 1 | 0.61629374 | 0.884845783 | −0.023517235 | 1 |
| ENSG00000105357 | −0.105194384 | 1 | −0.286257636 | 0.937722117 | 0.12225136 | 1 |
| ENSG00000105392 | −0.140180995 | 1 | 1.350403461 | 0.782613897 | 0.12132333 | 1 |
| ENSG00000105549 | −0.675865699 | 1 | 0.43424405 | 0.933776275 | −0.01546944 | 1 |
| ENSG00000105605 | −0.044046243 | 1 | −0.078930301 | 0.997233628 | −0.364251039 | 1 |
| ENSG00000105664 | 0.234783584 | 1 | 0.117592469 | 0.973212079 | −0.199203599 | 1 |
| ENSG00000106078 | −0.596349691 | 1 | 1.300744474 | 0.751130915 | 0.097709932 | 1 |
| ENSG00000106304 | −0.454358675 | 1 | 1.278780312 | 0.800133595 | 0.772079094 | 1 |
| ENSG00000106648 | −0.116446058 | 1 | 1.570956079 | 0.742949496 | 0.243774247 | 1 |
| ENSG00000106689 | 0.428190081 | 1 | −0.075171984 | 0.995803452 | 0.046815551 | 1 |
| ENSG00000107295 | −0.724258486 | 1 | 2.464703425 | 0.68994144 | −0.086266666 | 1 |
| ENSG00000107807 | 0.137827715 | 1 | 0.544587691 | 0.91119798 | 0.231211497 | 1 |
| ENSG00000108018 | −0.547468752 | 1 | 0.85269341 | 0.843439811 | 0.729297049 | 1 |
| ENSG00000109101 | 0.538410893 | 1 | −0.082464276 | 0.99353942 | −0.472244905 | 1 |
| ENSG00000110786 | 0.191502598 | 1 | 0.06896957 | 0.997155152 | −0.238985372 | 1 |
| ENSG00000110887 | 0.131111108 | 1 | −0.141498179 | 0.979953114 | 0.597447393 | 1 |
| ENSG00000110975 | −0.281499943 | 1 | −0.31391037 | 0.948383384 | −0.965079899 | 1 |
| ENSG00000111262 | −0.466826427 | 1 | 1.142878791 | 0.806478894 | 0.642772917 | 1 |
| ENSG00000111799 | −0.141911997 | 1 | 0.782138184 | 0.832139161 | 0.238185895 | 1 |
| ENSG00000112186 | −0.024260504 | 1 | 0.733866999 | 0.867584145 | 0.324860966 | 1 |
| ENSG00000112238 | 0.393170104 | 1 | 0.320987557 | 0.953891446 | 1.15377245 | 1 |
| ENSG00000112337 | −0.333294833 | 1 | 0.738233667 | 0.881825991 | 0.684826788 | 1 |
| ENSG00000112499 | −0.305955711 | 1 | 0.000100383 | 1 | 0.402578655 | 1 |
| ENSG00000114019 | 0.119358618 | 1 | 0.925741653 | 0.7871604 | −0.086135098 | 1 |
| ENSG00000115041 | −9.28E−06 | 1 | −0.126754251 | 0.980723343 | 0.215502642 | 1 |
| ENSG00000115155 | 0.197843374 | 1 | −0.204193956 | 0.934184441 | −0.265365542 | 1 |
| ENSG00000115648 | −0.639406576 | 1 | −0.152231941 | 0.978305518 | 0.334001525 | 1 |
| ENSG00000116176 | 0.76791399 | 1 | −1.73419835 | 0.661329741 | 0.133366927 | 1 |
| ENSG00000116721 | −0.261960328 | 1 | 2.477257959 | 0.726218814 | 0.947323758 | 1 |
| ENSG00000116748 | −0.261856016 | 1 | −0.621451573 | 0.885080104 | 0.613256343 | 1 |
| ENSG00000116833 | −0.312124377 | 1 | −0.114723584 | 0.991823026 | −0.210982797 | 1 |
| ENSG00000117114 | 0.580532431 | 1 | 1.383108381 | 0.725470307 | 0.382752752 | 1 |
| ENSG00000117501 | −0.242525931 | 1 | −0.356771873 | 0.923537499 | −0.094478934 | 1 |
| ENSG00000118194 | −0.194780553 | 1 | −0.045045649 | 1 | −0.028707775 | 1 |
| ENSG00000118733 | −0.168306891 | 1 | 0.457037361 | 0.92621162 | 0.311727154 | 1 |
| ENSG00000119125 | −0.247436947 | 1 | 0.000296093 | 1 | 0.467310042 | 1 |
| ENSG00000119283 | −0.304232098 | 1 | 0.867258927 | 0.818798586 | 0.26292956 | 1 |
| ENSG00000119547 | −0.24456357 | 1 | 1.784782986 | 0.723496808 | 0.103628364 | 1 |
| ENSG00000120251 | −0.09039807 | 1 | 1.488077153 | 0.724767358 | 0.041642117 | 1 |
| ENSG00000120332 | −0.518957551 | 1 | 0.742851998 | 0.858968348 | 0.159739349 | 1 |
| ENSG00000120738 | −0.092535102 | 1 | −1.368358877 | 0.627668963 | 0.333462255 | 1 |
| ENSG00000121207 | −0.195301435 | 1 | 0.231719596 | 0.969503557 | 0.01066207 | 1 |
| ENSG00000122121 | −0.109623373 | 1 | 0.864693822 | 0.777250738 | −0.249093554 | 1 |
| ENSG00000122778 | −0.127759628 | 1 | 0.206228029 | 0.951773266 | 0.218362019 | 1 |
| ENSG00000123243 | −0.505623699 | 1 | 1.247415012 | 0.73235655 | 0.195676108 | 1 |
| ENSG00000123572 | −0.203352014 | 1 | 0.683557554 | 0.88616233 | 0.605013071 | 1 |
| ENSG00000123977 | 0.488719569 | 1 | 1.503592086 | 0.787987908 | −0.29219265 | 1 |
| ENSG00000124092 | 0.002752214 | 1 | 0.80865262 | 0.840530754 | −0.019073803 | 1 |
| ENSG00000124253 | 0.27913906 | 1 | 0.276714095 | 0.961671576 | 0.284767529 | 1 |
| ENSG00000124440 | −0.273269415 | 1 | 1.369567585 | 0.762363148 | 0.14892327 | 1 |
| ENSG00000124466 | 0.289895975 | 1 | 0.763945067 | 0.841337914 | −0.097673747 | 1 |
| ENSG00000124749 | −0.514235619 | 1 | 1.41316074 | 0.762693024 | 0.092662892 | 1 |
| ENSG00000125255 | −0.027822112 | 1 | 0.044489879 | 1 | 0.555402909 | 1 |
| ENSG00000125492 | 0.879841413 | 1 | 0.899120028 | 0.855882707 | −0.754557485 | 1 |
| ENSG00000125740 | −0.208014548 | 1 | −0.722999194 | 0.725297356 | −0.122863284 | 1 |
| ENSG00000127129 | 0.552586146 | 1 | 0.492127351 | 0.930180704 | −0.351566411 | 1 |
| ENSG00000128573 | −0.163662169 | 1 | 1.204504792 | 0.787794769 | −0.19108042 | 1 |
| ENSG00000128917 | −0.165547246 | 1 | −0.27626558 | 0.951033674 | 0.211965616 | 1 |
| ENSG00000129946 | 0.430826667 | 1 | 0.986068391 | 0.754933936 | 0.443270016 | 1 |
| ENSG00000129990 | −0.01528682 | 1 | 1.631732813 | 0.745077552 | −0.061149728 | 1 |
| ENSG00000130045 | 0.144594627 | 1 | −0.278497988 | 0.946665677 | 0.334270957 | 1 |
| ENSG00000130226 | −0.491132363 | 1 | 0.400122019 | 0.917154157 | 0.04935684 | 1 |
| ENSG00000130287 | 0.316905174 | 1 | 0.420188275 | 0.920717063 | −0.642008684 | 1 |
| ENSG00000130477 | −0.041809609 | 1 | 0.394154009 | 0.905148092 | −0.28674943 | 1 |
| ENSG00000130508 | −0.472985684 | 1 | 0.226774267 | 0.954203705 | −0.131172062 | 1 |
| ENSG00000130528 | 0.225433939 | 1 | 1.996649839 | 0.722740931 | −0.363636866 | 1 |
| ENSG00000130540 | 0.111638129 | 1 | 2.178253808 | 0.722217784 | 0.071276285 | 1 |
| ENSG00000130635 | −0.011150205 | 1 | −0.056854342 | 0.990946263 | 0.076799389 | 1 |
| ENSG00000131044 | −0.193372839 | 1 | 0.74800611 | 0.828000591 | 0.086100403 | 1 |
| ENSG00000131183 | 0.132968094 | 1 | −0.764903911 | 0.832983886 | 0.131728528 | 1 |
| ENSG00000131386 | −0.02465071 | 1 | −0.275997847 | 0.960788648 | 0.115571653 | 1 |
| ENSG00000132297 | −0.45057748 | 1 | 0.335685986 | 0.946406426 | 0.544916255 | 1 |
| ENSG00000132321 | −0.329452064 | 1 | 0.488440298 | 0.910756217 | 0.375351855 | 1 |
| ENSG00000132639 | −0.408565796 | 1 | 1.905967427 | 0.726386579 | 0.425545535 | 1 |
| ENSG00000132972 | −0.231290586 | 1 | 0.046000411 | 1 | 0.196976455 | 1 |
| ENSG00000132975 | −0.229059585 | 1 | 0.860729873 | 0.878558735 | 0.035899532 | 1 |
| ENSG00000133083 | −0.20329481 | 1 | 0.453081712 | 0.908551976 | 0.023848382 | 1 |
| ENSG00000133110 | −0.34938544 | 1 | −0.086735544 | 0.992429838 | −0.345362803 | 1 |
| ENSG00000133124 | −0.72052378 | 1 | 5.343239343 | 0.627668963 | 0.002275328 | 1 |
| ENSG00000133454 | −0.273389213 | 1 | 0.397760747 | 0.892320091 | 0.190816003 | 1 |
| ENSG00000134240 | 0.097873964 | 1 | −0.238140625 | 0.963217892 | 0.024389112 | 1 |
| ENSG00000134871 | 0.140639269 | 1 | −0.412550673 | 0.850938998 | −0.091086456 | 1 |
| ENSG00000135097 | −0.20336144 | 1 | 0.497311089 | 0.937722117 | −0.05653922 | 1 |
| ENSG00000135409 | 0.204280771 | 1 | −0.275581101 | 0.956707132 | −0.438149966 | 1 |
| ENSG00000135454 | 0.299571806 | 1 | 0.64554358 | 0.867558968 | −0.208851705 | 1 |
| ENSG00000135472 | 0.202004682 | 1 | 0.818056333 | 0.844324446 | −0.2451995 | 1 |
| ENSG00000135917 | −0.392103408 | 1 | 1.852245487 | 0.724488969 | −0.403085239 | 1 |
| ENSG00000135931 | −0.131277483 | 1 | 0.804313724 | 0.819867485 | 0.374946273 | 1 |
| ENSG00000136352 | 1.366131821 | 1 | 1.260835582 | 0.799152726 | 0.316761789 | 1 |
| ENSG00000136535 | −0.306732856 | 1 | 1.986072677 | 0.725297356 | 0.298842535 | 1 |
| ENSG00000136574 | −0.085496591 | 1 | −0.352812935 | 0.937737382 | 0.61871766 | 1 |
| ENSG00000136695 | −0.281913586 | 1 | 0.461088841 | 0.936821983 | −0.051266805 | 1 |
| ENSG00000137203 | −0.53802252 | 1 | −0.281289526 | 0.966671784 | −0.833229666 | 1 |
| ENSG00000137573 | −0.087328771 | 1 | 0.825205342 | 0.843454046 | 0.191859508 | 1 |
| ENSG00000137648 | −0.536995553 | 1 | 1.979852135 | 0.698928831 | −0.833994499 | 1 |
| ENSG00000137766 | −0.800818915 | 1 | 0.083665679 | 0.991933875 | 0.528354497 | 1 |
| ENSG00000137819 | −0.26179588 | 1 | −0.822161664 | 1 | −0.158326194 | 1 |
| ENSG00000138162 | −0.394814157 | 1 | 0.898343482 | 0.988800576 | −0.138236443 | 1 |
| ENSG00000138650 | 0.014230206 | 1 | 0.227487401 | 0.96989385 | 0.891498883 | 1 |
| ENSG00000138675 | 0.89432515 | 1 | 1.669289062 | 0.754933936 | 0.390565845 | 1 |
| ENSG00000138759 | −0.238478196 | 1 | 0.713390438 | 0.828463489 | 0.168376931 | 1 |
| ENSG00000138892 | 0.314040376 | 1 | −0.418379815 | 0.92963834 | 0.177510233 | 1 |
| ENSG00000139144 | 0.299179692 | 1 | 1.417351311 | 0.754101584 | 0.429551463 | 1 |
| ENSG00000139220 | −0.068153108 | 1 | 0.235756127 | 0.956683515 | −0.19497989 | 1 |
| ENSG00000139767 | −0.194968482 | 1 | 0.895758938 | 0.818749045 | 0.114293744 | 1 |
| ENSG00000139865 | 0.138883348 | 1 | 0.239859825 | 0.964893129 | 0.44131884 | 1 |
| ENSG00000140279 | 0.347497874 | 1 | 0.455255659 | 0.901383337 | −0.862628479 | 1 |
| ENSG00000140527 | 0.120581454 | 1 | 0.32760164 | 0.945248895 | 0.342334255 | 1 |
| ENSG00000140600 | −0.050600896 | 1 | 0.773782722 | 0.86197857 | 0.265942486 | 1 |
| ENSG00000141434 | −0.274970362 | 1 | 1.075438474 | 0.801861697 | 0.454970747 | 1 |
| ENSG00000141668 | 0.075687803 | 1 | 1.63670017 | 0.761543823 | 0.84998264 | 1 |
| ENSG00000141946 | −0.09028345 | 1 | 0.08223031 | 0.996460723 | 0.119192786 | 1 |
| ENSG00000142408 | −0.003298868 | 1 | 0.659965868 | 0.750144627 | 0.047494152 | 1 |
| ENSG00000142449 | −0.470497888 | 1 | −0.030000224 | 1 | 0.827689481 | 1 |
| ENSG00000142549 | −0.00560726 | 1 | 0.73291036 | 0.889236595 | −0.165464006 | 1 |
| ENSG00000142611 | −0.211729678 | 1 | 0.541607842 | 0.876517598 | −0.117193595 | 1 |
| ENSG00000142623 | −0.168154589 | 1 | 0.648194613 | 0.880022596 | −0.307236278 | 1 |
| ENSG00000142675 | 0.281272134 | 1 | −0.617267919 | 0.812662354 | 0.640684158 | 1 |
| ENSG00000142910 | 0.16987273 | 1 | −0.809803802 | 0.814580181 | 0.016307454 | 1 |
| ENSG00000143107 | −0.167664787 | 1 | 1.06958764 | 0.789188535 | 0.167596527 | 1 |
| ENSG00000143355 | 0.142003448 | 1 | 2.142940272 | 0.665274436 | 0.633424086 | 1 |
| ENSG00000143450 | −0.004524812 | 1 | 0.151530747 | 0.977890656 | −0.286819852 | 1 |
| ENSG00000143469 | −0.447510986 | 1 | −0.956862615 | 0.814402007 | 0.457714936 | 1 |
| ENSG00000143867 | −0.581637838 | 1 | 1.277349941 | 0.762398021 | −0.24655505 | 1 |
| ENSG00000144115 | 0.096951122 | 1 | 1.406766298 | 0.627668963 | −0.893838536 | 1 |
| ENSG00000144488 | 0.12283529 | 1 | 0.119367304 | 0.984168271 | −0.472455254 | 1 |
| ENSG00000144648 | 0.031376673 | 1 | −0.013830853 | 1 | 0.289490869 | 1 |
| ENSG00000144681 | −0.063452505 | 1 | 2.442386048 | 0.655801493 | −0.190358616 | 1 |
| ENSG00000144730 | −0.325294976 | 1 | −0.187758677 | 0.965332255 | 0.08073396 | 1 |
| ENSG00000144820 | −0.373299237 | 1 | 1.054797057 | 0.807095259 | −0.865616935 | 1 |
| ENSG00000144908 | 0.017343053 | 1 | 0.613763367 | 0.869925678 | 0.034576065 | 1 |
| ENSG00000145242 | −0.091519359 | 1 | 0.912178704 | 0.85680137 | 0.146217144 | 1 |
| ENSG00000145526 | −0.802285847 | 1 | 1.000991412 | 0.843439811 | 0.179252073 | 1 |
| ENSG00000145642 | 0.273650832 | 1 | 0.854782399 | 0.897785147 | 1.025590395 | 1 |
| ENSG00000145832 | −0.280573959 | 1 | 1.513859271 | 0.770970718 | 0.10287202 | 1 |
| ENSG00000146005 | 0.021403858 | 1 | 0.480468991 | 0.905798309 | −0.110841717 | 1 |
| ENSG00000146521 | −0.374380824 | 1 | 1.066067068 | 0.841097873 | 0.248763858 | 1 |
| ENSG00000146648 | −0.33233532 | 1 | 0.951029589 | 0.797043771 | 0.222515844 | 1 |
| ENSG00000146839 | −0.125191008 | 1 | 0.079846162 | 0.992989633 | −0.139812795 | 1 |
| ENSG00000146950 | 0.212278038 | 1 | 0.779222778 | 0.849012793 | −0.08543539 | 1 |
| ENSG00000147573 | −0.594059397 | 1 | 1.095002077 | 0.799860378 | −0.162457891 | 1 |
| ENSG00000147655 | −0.332530076 | 1 | 1.217191102 | 0.794524132 | 0.50470898 | 1 |
| ENSG00000147689 | 0.16848171 | 1 | −0.903328622 | 0.77318391 | 0.088442978 | 1 |
| ENSG00000148357 | 0.264805329 | 1 | 1.320803031 | 0.732397555 | −0.197042982 | 1 |
| ENSG00000148408 | 0.048380959 | 1 | 0.530819495 | 0.874968309 | −0.030898439 | 1 |
| ENSG00000148513 | −0.305028977 | 1 | 0.953450117 | 0.839264767 | 0.371091001 | 1 |
| ENSG00000148702 | −0.70168564 | 1 | 0.898185199 | 0.891304198 | −0.267216198 | 1 |
| ENSG00000148342 | 0.589091502 | 1 | 2.021178416 | 0.724855033 | 0.288605968 | 1 |
| ENSG00000149633 | −0.002950944 | 1 | 0.845177883 | 0.841263195 | 0.020108457 | 1 |
| ENSG00000149654 | 0.418594264 | 1 | 1.842451251 | 0.704650879 | 0.373083932 | 1 |
| ENSG00000149926 | 0.338790838 | 1 | 0.83888801 | 0.87726176 | 0.223312443 | 1 |
| ENSG00000150086 | 0.101383742 | 1 | 0.08254319 | 0.9930323812 | 0.011920418 | 1 |
| ENSG00000150893 | −0.018022791 | 1 | 0.683991736 | 0.858353069 | 0.230897523 | 1 |
| ENSG00000151224 | 0.197853903 | 1 | 2.604050819 | 0.899487058 | 0.028446108 | 1 |
| ENSG00000151474 | 0.078484878 | 1 | −1.188547325 | 0.525549357 | 0.101780987 | 1 |
| ENSG00000151572 | −0.114230143 | 1 | 1.895495335 | 0.731198014 | 0.34018558 | 1 |
| ENSG00000152822 | 0.198572202 | 1 | 0.951055318 | 0.849012793 | 0.351777006 | 1 |
| ENSG00000152910 | −0.424881197 | 1 | 0.369465713 | 0.937722117 | −0.22837856 | 1 |
| ENSG00000153165 | −0.234684905 | 1 | 2.095569153 | 0.627668963 | −0.265417248 | 1 |
| ENSG00000154099 | 0.345094508 | 1 | 1.561795966 | 0.738962739 | −0.424945152 | 1 |
| ENSG00000154478 | −0.603462434 | 1 | 3.14725066 | 0.63231269 | 0.299935738 | 1 |
| ENSG00000155816 | −0.266666973 | 1 | −0.193230828 | 0.97215979 | 0.364876442 | 1 |
| ENSG00000156076 | −0.291654174 | 1 | −0.119157606 | 0.992675317 | 0.39962807 | 1 |
| ENSG00000156103 | 0.178584644 | 1 | 1.516003497 | 0.737781088 | 0.049120893 | 1 |
| ENSG00000156222 | 0.102974247 | 1 | −0.178812362 | 0.971374933 | 0.083114459 | 1 |
| ENSG00000156413 | 0.04072306 | 1 | 1.918694434 | 0.760351327 | −0.126102335 | 1 |
| ENSG00000156687 | −0.175857955 | 1 | −0.04484555 | 1 | 0.372334105 | 1 |
| ENSG00000157214 | −0.138333013 | 1 | 3.283188698 | 0.627668963 | 0.1941259 | 1 |
| ENSG00000157423 | −0.307482345 | 1 | 0.771550118 | 0.824915096 | 0.345889545 | 1 |
| ENSG00000157927 | 0.749636954 | 1 | 0.477550274 | 0.905148092 | −0.15547528 | 1 |
| ENSG00000158077 | 0.068140817 | 1 | 0.259016203 | 0.966311339 | 0.259994409 | 1 |
| ENSG00000158125 | −0.505018354 | 1 | 1.290307308 | 0.756996807 | 0.187029284 | 1 |
| ENSG00000158258 | −0.439359401 | 1 | 1.26103511 | 0.752976057 | 0.225413257 | 1 |
| ENSG00000159251 | −0.361085345 | 1 | 1.515815689 | 0.749009777 | 0.188962838 | 1 |
| ENSG00000159337 | 0.452174015 | 1 | 0.95816216 | 0.824640952 | −0.148848276 | 1 |
| ENSG00000159650 | −0.050043865 | 1 | 1.288104367 | 0.750144627 | −0.172852982 | 1 |
| ENSG00000160460 | 0.000418346 | 1 | 0.227969932 | 0.95541991 | −0.204422386 | 1 |
| ENSG00000160994 | 0.124559299 | 1 | 4.22482034 | 0.676125287 | 0.347165754 | 1 |
| ENSG00000161103 | −0.608562688 | 1 | 1.675951587 | 0.727990986 | 0.130918148 | 1 |
| ENSG00000161243 | −0.622240471 | 1 | 2.046982131 | 0.708026694 | 0.793324098 | 1 |
| ENSG00000161270 | −0.677560856 | 1 | −0.316527889 | 0.931527297 | −0.164586694 | 1 |
| ENSG00000161609 | 0.321956534 | 1 | 1.27376202 | 0.776886286 | −0.254407597 | 1 |
| ENSG00000161682 | 0.26231244 | 1 | 0.789426772 | 0.876517598 | 0.081403063 | 1 |
| ENSG00000161940 | 0.306629089 | 1 | 1.015597853 | 0.820668118 | 0.229086895 | 1 |
| ENSG00000162006 | 1.067686879 | 1 | 0.162688447 | 0.974864202 | −0.300137609 | 1 |
| ENSG00000162062 | 0.450095579 | 1 | −0.04152343 | 1 | 0.295152575 | 1 |
| ENSG00000162510 | −0.101984244 | 1 | −0.576819273 | 0.854897053 | 0.272474864 | 1 |
| ENSG00000162631 | −0.401679035 | 1 | 0.274332455 | 0.948978235 | 0.539361847 | 1 |
| ENSG00000162706 | 0.371300869 | 1 | −0.555096936 | 0.875275045 | 0.057571537 | 1 |
| ENSG00000162738 | −0.114701686 | 1 | 0.109931908 | 0.985019079 | −0.152715741 | 1 |
| ENSG00000163395 | −0.487480191 | 1 | 0.683264747 | 0.849012793 | 0.200522973 | 1 |
| ENSG00000163689 | −0.216411226 | 1 | 1.102148208 | 0.773447218 | 0.232500831 | 1 |
| ENSG00000163914 | −0.713160021 | 1 | 0.415253755 | 0.915153867 | −0.848474498 | 1 |
| ENSG00000163975 | 0.159834765 | 1 | −0.251120274 | 0.933291917 | 0.020651785 | 1 |
| ENSG00000163995 | −0.105526095 | 1 | 0.517085722 | 0.773755142 | 0.233221699 | 1 |
| ENSG00000164093 | 0.083391252 | 1 | 0.11939448 | 0.984850234 | 0.039253213 | 1 |
| ENSG00000164107 | −0.314024481 | 1 | 5.380618532 | 0.627668963 | 0.16433772 | 1 |
| ENSG00000164122 | 0.035211823 | 1 | 1.371358167 | 0.769405357 | −0.088086419 | 1 |
| ENSG00000164265 | −1.113984543 | 1 | 0.404356031 | 0.910756217 | 0.088267228 | 1 |
| ENSG00000164294 | −0.219928588 | 1 | 2.094690441 | 0.725843889 | 0.279466544 | 1 |
| ENSG00000164393 | −0.075720464 | 1 | 1.22944626 | 0.794241038 | 0.276302456 | 1 |
| ENSG00000164692 | −0.11373057 | 1 | −0.192726392 | 0.949746132 | 0.114611589 | 1 |
| ENSG00000164694 | −0.150125663 | 1 | 0.35115973 | 0.91831034 | 0.00936716 | 1 |
| ENSG00000164904 | −0.45949042 | 1 | 0.519367578 | 0.857601604 | −0.173250351 | 1 |
| ENSG00000165300 | 0.204525242 | 1 | 0.567551507 | 0.899216003 | −0.567934478 | 1 |
| ENSG00000165323 | −0.169846908 | 1 | 0.929543197 | 0.819610241 | 0.111656038 | 1 |
| ENSG00000165379 | −0.357859717 | 1 | 0.992563648 | 0.854965024 | 0.11024127 | 1 |
| ENSG00000165495 | −0.364534906 | 1 | 2.475038628 | 0.627668963 | 0.013822717 | 1 |
| ENSG00000165566 | −0.32625258 | 1 | 2.213746235 | 0.6989448 | −0.055804664 | 1 |
| ENSG00000165757 | −0.072388796 | 1 | −0.743888409 | 0.791276686 | 0.488556874 | 1 |
| ENSG00000165816 | −0.177404904 | 1 | −0.171170948 | 0.977382049 | 0.345080658 | 1 |
| ENSG00000165966 | −0.285064897 | 1 | 0.808503061 | 0.853575685 | 0.178661123 | 1 |
| ENSG00000165970 | −0.374632483 | 1 | 0.402947708 | 0.939106996 | 0.607693159 | 1 |
| ENSG00000165973 | −0.107894805 | 1 | 0.550641336 | 0.913766513 | 0.368089989 | 1 |
| ENSG00000166118 | 0.106439727 | 1 | 0.773848003 | 0.865676998 | 0.035244334 | 1 |
| ENSG00000166159 | 0.379309904 | 1 | 0.725763036 | 0.871961527 | −0.081415978 | 1 |
| ENSG00000166292 | −0.406445456 | 1 | 5.310152728 | 0.627668963 | 0.517986656 | 1 |
| ENSG00000166391 | −0.609893138 | 1 | 0.12785563 | 0.98144518 | 0.445024166 | 1 |
| ENSG00000166444 | −0.205220199 | 1 | −0.270219766 | 0.919970837 | −0.025992347 | 1 |
| ENSG00000166473 | 0.068479882 | 1 | 0.803294803 | 0.825562858 | 0.035379592 | 1 |
| ENSG00000166558 | 0.128492019 | 1 | 0.461031781 | 0.922604678 | 0.143250087 | 1 |
| ENSG00000166596 | −0.715740917 | 1 | 0.969349716 | 0.827351425 | −0.073518986 | 1 |
| ENSG00000166689 | −0.204146306 | 1 | 0.656450403 | 0.733544265 | −0.086057303 | 1 |
| ENSG00000166863 | 0.278320889 | 1 | 2.053024881 | 0.701331844 | 0.32256963 | 1 |
| ENSG00000166869 | −0.353841317 | 1 | 0.055580641 | 0.999535068 | 0.321322593 | 1 |
| ENSG00000166984 | −0.437351638 | 1 | −0.57447992 | 0.867584145 | −0.212881731 | 1 |
| ENSG00000167654 | 0.929551262 | 1 | 2.683854099 | 0.656365383 | 0.000422142 | 1 |
| ENSG00000167723 | −0.152316762 | 1 | 0.399307054 | 0.88017288 | 0.051736039 | 1 |
| ENSG00000167757 | 0.107732329 | 1 | 2.144333068 | 0.741966212 | 0.041152411 | 1 |
| ENSG00000167779 | 0.419440126 | 1 | 0.84301591 | 0.840244238 | −0.049067284 | 1 |
| ENSG00000167798 | −0.691807116 | 1 | 1.232436905 | 0.794524132 | 0.047298354 | 1 |
| ENSG00000168077 | 0.155387518 | 1 | 0.283630465 | 0.945347234 | −0.031270417 | 1 |
| ENSG00000168079 | 0.304909506 | 1 | 0.842542483 | 0.79237154 | 0.109013922 | 1 |
| ENSG00000168356 | −0.610944587 | 1 | −0.263924959 | 0.944798487 | 0.35010504 | 1 |
| ENSG00000168367 | −0.593669591 | 1 | 1.742787077 | 0.741059294 | 0.058845896 | 1 |
| ENSG00000168481 | −0.177680012 | 1 | −0.176057427 | 0.975121981 | −0.361393393 | 1 |
| ENSG00000168484 | −0.55159713 | 1 | −0.306770833 | 0.950911989 | 0.177687701 | 1 |
| ENSG00000168542 | 0.063619984 | 1 | −0.366211553 | 0.905049562 | −0.25532392 | 1 |
| ENSG00000168907 | −0.34434884 | 1 | 0.027761324 | 1 | 0.095854026 | 1 |
| ENSG00000168959 | −0.377363911 | 1 | 0.590871081 | 0.882007771 | 0.084617918 | 1 |
| ENSG00000169126 | −0.488364348 | 1 | 1.815920531 | 0.732815758 | 0.904302929 | 1 |
| ENSG00000169169 | 0.631079672 | 1 | −0.095612355 | 0.98993439 | −0.345407026 | 1 |
| ENSG00000169344 | −0.135743663 | 1 | 0.408154074 | 0.931976942 | −0.175511803 | 1 |
| ENSG00000169436 | −0.280059254 | 1 | 0.747486195 | 0.821188312 | 0.140488858 | 1 |
| ENSG00000169862 | −0.027224035 | 1 | 0.680129393 | 0.841176646 | 0.284817064 | 1 |
| ENSG00000169876 | 0.684807971 | 1 | 1.393206012 | 0.747487389 | 0.615964856 | 1 |
| ENSG00000170381 | 0.017568151 | 1 | 0.396729287 | 0.943406874 | 0.074345074 | 1 |
| ENSG00000170426 | −0.50740439 | 1 | 0.508730318 | 0.915138245 | 0.170350996 | 1 |
| ENSG00000170442 | 0.675246177 | 1 | 0.47253793 | 0.911690397 | −0.700899984 | 1 |
| ENSG00000170703 | −0.096172488 | 1 | 0.735266138 | 0.869343886 | 0.301559858 | 1 |
| ENSG00000170777 | −0.087908478 | 1 | −0.189830775 | 0.973108836 | 0.614118673 | 1 |
| ENSG00000170927 | −0.285697631 | 1 | 0.248532627 | 0.954182234 | 0.046041883 | 1 |
| ENSG00000171435 | −0.366228855 | 1 | 0.311957308 | 0.943590841 | 0.31694928 | 1 |
| ENSG00000171487 | −0.770914894 | 1 | 0.115434887 | 0.987361879 | 0.085527802 | 1 |
| ENSG00000171533 | 0.090811472 | 1 | 1.502755849 | 0.722328586 | 0.137119381 | 1 |
| ENSG00000171564 | −0.849314018 | 1 | 0.314686491 | 0.948978235 | 0.333409588 | 1 |
| ENSG00000171587 | −0.580745445 | 1 | −0.030879098 | 1 | 0.195798971 | 1 |
| ENSG00000171804 | −0.682786048 | 1 | 0.444774723 | 0.909888442 | 0.373158383 | 1 |
| ENSG00000172137 | 0.217779933 | 1 | 2.09457805 | 0.708026694 | −0.206290856 | 1 |
| ENSG00000172350 | 0.314257023 | 1 | 1.422837709 | 0.784917559 | −0.1292138 | 1 |
| ENSG00000172752 | −0.407459921 | 1 | 0.034008655 | 1 | 0.391718527 | 1 |
| ENSG00000172900 | −0.075673221 | 1 | 0.242189975 | 0.989429801 | −0.168178485 | 1 |
| ENSG00000172995 | −0.454127275 | 1 | 1.644518848 | 0.729803191 | −0.254249133 | 1 |
| ENSG00000173013 | 0.507438345 | 1 | 0.129993358 | 0.971571073 | −0.352824138 | 1 |
| ENSG00000173227 | 0.610713148 | 1 | 0.81206281 | 0.844324446 | −0.328292543 | 1 |
| ENSG00000173572 | 0.131321529 | 1 | −0.470050345 | 0.905836541 | 0.425559982 | 1 |
| ENSG00000173702 | −0.528206461 | 1 | 1.168800706 | 0.794618344 | 0.833393032 | 1 |
| ENSG00000173769 | −0.47844312 | 1 | −0.338583882 | 0.938972292 | 1.035489015 | 1 |
| ENSG00000173826 | 0.394938306 | 1 | 1.00529897 | 0.828704895 | −0.56022732 | 1 |
| ENSG00000174279 | −0.57084984 | 1 | 0.799078553 | 0.889485378 | 0.127210495 | 1 |
| ENSG00000174358 | 0.13920852 | 1 | −0.774625877 | 0.768821837 | 0.337622528 | 1 |
| ENSG00000174498 | −0.508913157 | 1 | 1.888251821 | 0.731198014 | −0.167547523 | 1 |
| ENSG00000174502 | −0.291131487 | 1 | 0.887488589 | 0.881825991 | −0.082688663 | 1 |
| ENSG00000174963 | 0.015294792 | 1 | 0.73795342 | 0.868736761 | −0.42571763 | 1 |
| ENSG00000175084 | 0.246561209 | 1 | 1.769951268 | 0.686547614 | −0.118496503 | 1 |
| ENSG00000175267 | −0.162720501 | 1 | 0.187461706 | 0.958051922 | 0.05746252 | 1 |
| ENSG00000175267 | −0.162720501 | 1 | 0.187461706 | 0.958051922 | 0.05746252 | 1 |
| ENSG00000175329 | −0.551353963 | 1 | 1.201778078 | 0.811501172 | −0.735778955 | 1 |
| ENSG00000175535 | −0.389366106 | 1 | 0.958606296 | 0.850895463 | 0.152818215 | 1 |
| ENSG00000176584 | 0.264698304 | 1 | 1.619688013 | 0.744348791 | −0.027915483 | 1 |
| ENSG00000176769 | −0.23701654 | 1 | 0.482999116 | 0.923307954 | 0.07382279 | 1 |
| ENSG00000177045 | 0.346214135 | 1 | −0.883712439 | 0.810351831 | 0.138418184 | 1 |
| ENSG00000177103 | 0.366761309 | 1 | 0.150265906 | 0.967944135 | −0.149492038 | 1 |
| ENSG00000177354 | 0.354977192 | 1 | −0.247044947 | 0.955305328 | 0.2797613 | 1 |
| ENSG00000177511 | 0.055097244 | 1 | 2.626082561 | 0.687959091 | 0.32761642 | 1 |
| ENSG00000177551 | 0.06194014 | 1 | 6.104847404 | 0.627668963 | 0.000430134 | 1 |
| ENSG00000178031 | −0.209982696 | 1 | 1.139083059 | 0.786877233 | 0.220809557 | 1 |
| ENSG00000178171 | 0.229558372 | 1 | 1.012029291 | 0.800489011 | 0.163647987 | 1 |
| ENSG00000178568 | −0.189812542 | 1 | 0.875337112 | 0.828433164 | 0.142857609 | 1 |
| ENSG00000178645 | −0.131400788 | 1 | −0.029554337 | 1 | 0.592068389 | 1 |
| ENSG00000178965 | −0.463966108 | 1 | 1.267106841 | 0.755765503 | 0.225343111 | 1 |
| ENSG00000179008 | −0.360701751 | 1 | 1.892568105 | 0.755052726 | −0.209200201 | 1 |
| ENSG00000179178 | 0.036818661 | 1 | 0.399238903 | 0.939106996 | 0.45023468 | 1 |
| ENSG00000179270 | −0.016581454 | 1 | 1.212207623 | 0.773820479 | −0.018033757 | 1 |
| ENSG00000179709 | −0.183052797 | 1 | −0.638700795 | 0.876029113 | 0.499537451 | 1 |
| ENSG00000179766 | −0.483317498 | 1 | 2.675052137 | 0.656599592 | 0.080758193 | 1 |
| ENSG00000179813 | −0.066443991 | 1 | 1.117423548 | 0.855882707 | 0.511048103 | 1 |
| ENSG00000180251 | 0.354132333 | 1 | 1.853862248 | 0.729803191 | 0.137552884 | 1 |
| ENSG00000181143 | 0.265140388 | 1 | −0.610345242 | 0.829802444 | 0.370527471 | 1 |
| ENSG00000181355 | −0.661189174 | 1 | 0.495367178 | 0.913357086 | −0.37543806 | 1 |
| ENSG00000181378 | −0.142589287 | 1 | −0.442095435 | 0.885310895 | −0.040868213 | 1 |
| ENSG00000182256 | 0.00161351 | 1 | 1.678376167 | 0.747180575 | 0.537162329 | 1 |
| ENSG00000183016 | −0.409869712 | 1 | 0.204059907 | 0.963151217 | 0.164903336 | 1 |
| ENSG00000183067 | 0.011001437 | 1 | 5.552457083 | 0.627668963 | 0.610657118 | 1 |
| ENSG00000183206 | 0.288269994 | 1 | 1.44511872 | 0.771574746 | 0.091740315 | 1 |
| ENSG00000183242 | −0.249011535 | 1 | 2.831419092 | 0.689965785 | 0.221993402 | 1 |
| ENSG00000183287 | −0.108476557 | 1 | 0.677199626 | 0.88616233 | 0.102473963 | 1 |
| ENSG00000183304 | 0.782981855 | 1 | 1.285285772 | 0.817952528 | −0.064655496 | 1 |
| ENSG00000183317 | −0.320481618 | 1 | 0.884261146 | 0.820489984 | 0.119204369 | 1 |
| ENSG00000183580 | −0.524894327 | 1 | 0.925469737 | 0.860534878 | 0.671373557 | 1 |
| ENSG00000183668 | −0.250813779 | 1 | −0.395330668 | 0.939971429 | 0.58541285 | 1 |
| ENSG00000183778 | −0.981318157 | 1 | 2.317782659 | 0.722505857 | 0.663167198 | 1 |
| ENSG00000183780 | 0.083035636 | 1 | 0.842283744 | 0.692730659 | −0.242552512 | 1 |
| ENSG00000183856 | −0.18178081 | 1 | −0.438153854 | 0.859665429 | 0.160670003 | 1 |
| ENSG00000183876 | 0.27277532 | 1 | 1.28888811 | 0.787336888 | −0.216444352 | 1 |
| ENSG00000183908 | 0.045614564 | 1 | 2.382699282 | 0.713135474 | 0.020004922 | 1 |
| ENSG00000184012 | −0.246437402 | 1 | 0.100566228 | 0.991823026 | −0.08984279 | 1 |
| ENSG00000184227 | 0.52223814 | 1 | −0.626385273 | 0.794524132 | −0.412718998 | 1 |
| ENSG00000184302 | 0.558283399 | 1 | −0.045856923 | 1 | 0.231187888 | 1 |
| ENSG00000184304 | −0.063709292 | 1 | 1.432135626 | 0.74753626 | 0.035035921 | 1 |
| ENSG00000184809 | 0.140091076 | 1 | −0.05616504 | 0.99937713 | 0.744088602 | 1 |
| ENSG00000184908 | 0.402320282 | 1 | −0.836321916 | 0.799148915 | 0.134333106 | 1 |
| ENSG00000185038 | −0.192967031 | 1 | 0.867416789 | 0.82438292 | −0.115923539 | 1 |
| ENSG00000185069 | −0.624748239 | 1 | −0.69532548 | 0.855426705 | −0.328889771 | 1 |
| ENSG00000185303 | −0.404866654 | 1 | 2.140859914 | 0.727990986 | 0.30194417 | 1 |
| ENSG00000185313 | −0.456626467 | 1 | 0.167482957 | 0.973318649 | 0.143189125 | 1 |
| ENSG00000185467 | −0.275420291 | 1 | −0.30817587 | 0.948256578 | 0.279772356 | 1 |
| ENSG00000185686 | −0.341640386 | 1 | 0.328835819 | 0.941835771 | 0.819682549 | 1 |
| ENSG00000185737 | −0.587511457 | 1 | −0.202080434 | 0.974602748 | 0.397111249 | 1 |
| ENSG00000185739 | −0.411889582 | 1 | 1.10110047 | 0.794748396 | 0.040317772 | 1 |
| ENSG00000185823 | −0.22845567 | 1 | 0.31798295 | 0.939781849 | 0.780350439 | 1 |
| ENSG00000185974 | 0.327606152 | 1 | 0.841577371 | 0.815513218 | 0.105802313 | 1 |
| ENSG00000186185 | 0.134590856 | 1 | 1.659755026 | 0.627668963 | −0.043354427 | 1 |
| ENSG00000186393 | −0.574609347 | 1 | −0.273830863 | 0.967516048 | −0.245635919 | 1 |
| ENSG00000186487 | −0.24557519 | 1 | 1.169653359 | 0.773755142 | 0.273450958 | 1 |
| ENSG00000186526 | −0.291164062 | 1 | 0.194917069 | 0.976008317 | 0.046648148 | 1 |
| ENSG00000186862 | 0.247136803 | 1 | 0.223054668 | 0.933489898 | 0.072854071 | 1 |
| ENSG00000187068 | −0.165105326 | 1 | −0.093299772 | 0.991823026 | 0.304445715 | 1 |
| ENSG00000187094 | 1.13454822 | 1 | 0.058939969 | 1 | −0.258457511 | 1 |
| ENSG00000187123 | −0.57357905 | 1 | 0.866131631 | 0.820682968 | 0.56627688 | 1 |
| ENSG00000187527 | −0.41275252 | 1 | 1.198866564 | 0.786877233 | 0.279380903 | 1 |
| ENSG00000187772 | 0.216214401 | 1 | −0.019347151 | 1 | 0.066213621 | 1 |
| ENSG00000187905 | −0.031748982 | 1 | 1.2449596 | 0.78513512 | 0.049322593 | 1 |
| ENSG00000187955 | −0.101927235 | 1 | 0.493543 | 0.886962045 | 0.093906397 | 1 |
| ENSG00000187997 | 0.090918915 | 1 | −0.722809509 | 0.77316391 | 0.292911541 | 1 |
| ENSG00000188086 | −0.219432299 | 1 | 2.05684047 | 0.714131936 | 0.081588504 | 1 |
| ENSG00000188112 | −0.309851842 | 1 | 1.835030288 | 0.697833232 | −0.543290658 | 1 |
| ENSG00000188162 | −0.004039957 | 1 | 0.595459788 | 0.858968348 | 0.031693298 | 1 |
| ENSG00000188338 | −0.516375224 | 1 | 0.913485421 | 0.813117296 | 0.112725863 | 1 |
| ENSG00000188488 | −0.398627099 | 1 | −0.066986093 | 0.99928282 | 0.034654676 | 1 |
| ENSG00000188706 | 0.055051422 | 1 | 0.707442896 | 0.705378609 | 0.084976724 | 1 |
| ENSG00000188782 | 0.376162979 | 1 | −0.078094345 | 0.995680337 | 0.022695997 | 1 |
| ENSG00000188803 | 0.142254216 | 1 | 0.753257185 | 0.861319653 | 0.190594903 | 1 |
| ENSG00000188886 | −0.606540007 | 1 | 0.949025586 | 0.781067562 | −0.021126839 | 1 |
| ENSG00000196091 | −0.626720148 | 1 | 0.309888777 | 0.933370884 | −0.010414474 | 1 |
| ENSG00000196136 | −0.356107603 | 1 | 0.191407925 | 0.97700852 | −0.282370334 | 1 |
| ENSG00000196361 | 0.301485434 | 1 | 1.953137262 | 0.706499236 | −0.236289109 | 1 |
| ENSG00000196415 | −0.301671112 | 1 | 1.102437354 | 0.834571279 | −0.376208763 | 1 |
| ENSG00000196565 | −0.512995497 | 1 | −0.260328339 | 0.915138245 | −0.54792429 | 1 |
| ENSG00000196862 | −0.37604602 | 1 | 6.58900869 | 0.402387206 | 0.140028962 | 1 |
| ENSG00000197079 | −0.090695231 | 1 | 2.158446723 | 0.697833232 | −0.632012814 | 1 |
| ENSG00000197085 | −0.320906913 | 1 | 6.213739468 | 0.627668963 | 0.689886278 | 1 |
| ENSG00000197406 | 0.404297609 | 1 | 0.463641897 | 0.935206684 | −0.223986968 | 1 |
| ENSG00000197408 | −0.63253654 | 1 | 0.902342099 | 0.837574131 | −0.552716613 | 1 |
| ENSG00000197893 | −0.271589128 | 1 | 0.792601765 | 0.853462632 | 0.153136007 | 1 |
| ENSG00000197991 | 0.070232594 | 1 | −0.413722368 | 0.916194501 | −0.158852377 | 1 |
| ENSG00000198010 | 0.068714495 | 1 | 1.089410281 | 0.791327924 | 0.195394349 | 1 |
| ENSG00000198597 | −0.445240194 | 1 | 0.631864973 | 0.872919273 | 0.365147069 | 1 |
| ENSG00000198765 | 0.018200108 | 1 | −0.44543802 | 0.913883645 | 0.593151793 | 1 |
| ENSG00000198788 | 0.289211194 | 1 | 0.760795909 | 0.824640952 | −0.243937903 | 1 |
| ENSG00000198914 | −0.01913062 | 1 | 2.501317942 | 0.707562018 | 0.163345574 | 1 |
| ENSG00000198929 | −0.482658569 | 1 | 2.16815026 | 0.640562142 | 0.142426268 | 1 |
| ENSG00000203805 | −0.402456377 | 1 | −0.697779625 | 0.843135524 | −0.146576451 | 1 |
| ENSG00000203900 | 0.236165065 | 1 | −0.168928987 | 0.97893783 | 0.297866836 | 1 |
| ENSG00000204055 | −0.010051371 | 1 | 0.074072995 | 0.992895239 | −0.246765108 | 1 |
| ENSG00000204241 | −0.232933883 | 1 | 0.294013453 | 0.908182547 | 0.165068872 | 1 |
| ENSG00000204283 | −0.228184457 | 1 | 0.631351983 | 0.869751179 | 0.435875025 | 1 |
| ENSG00000204335 | −0.693704258 | 1 | 3.818503481 | 0.654405468 | −0.629007298 | 1 |
| ENSG00000204624 | 0.072435123 | 1 | 0.368182964 | 0.908517399 | −0.012012324 | 1 |
| ENSG00000204661 | 0.228008501 | 1 | 1.464324816 | 0.818753547 | 0.320839584 | 1 |
| ENSG00000204929 | −0.866338929 | 1 | 0.579137938 | 0.896714922 | 0.775627303 | 1 |
| ENSG00000204941 | −0.013942807 | 1 | 0.234390505 | 0.969012561 | 0.627451691 | 1 |
| ENSG00000205054 | −1.112595252 | 1 | 1.432381363 | 0.762363148 | 0.219446559 | 1 |
| ENSG00000205176 | −0.149338523 | 1 | 1.703370547 | 0.729530842 | 0.03289074 | 1 |
| ENSG00000205312 | −0.895602346 | 1 | 4.115403356 | 0.627668963 | −0.339982563 | 1 |
| ENSG00000205396 | −0.726364765 | 1 | −0.158146417 | 0.979462675 | 0.016687252 | 1 |
| ENSG00000205592 | −0.434301209 | 1 | 0.984766625 | 0.787682192 | 0.298495845 | 1 |
| ENSG00000205667 | −0.055380476 | 1 | 4.33427616 | 0.634030703 | 0.001757458 | 1 |
| ENSG00000205922 | −0.585676548 | 1 | −0.724310049 | 0.849012793 | −0.305046492 | 1 |
| ENSG00000205976 | 0.261966231 | 1 | 0.948880524 | 0.86678933 | −0.382219884 | 1 |
| ENSG00000210127 | −0.731653603 | 1 | 0.560715026 | 0.861305803 | −0.161520037 | 1 |
| ENSG00000213467 | 0.64533241 | 1 | 5.32914163 | 0.627668963 | 0.131234386 | 1 |
| ENSG00000213864 | 0.09851276 | 1 | 0.671113195 | 0.884405435 | 0.368107814 | 1 |
| ENSG00000213934 | 0.51786763 | 1 | −1.313099497 | 0.687959091 | −0.59732425 | 1 |
| ENSG00000214128 | 0.013626188 | 1 | 0.834709087 | 0.852992821 | 0.012681621 | 1 |
| ENSG00000214181 | −0.02441144 | 1 | 1.472777831 | 0.681217684 | 0.544010143 | 1 |
| ENSG00000214402 | 0.314446807 | 1 | 1.403831004 | 0.741059294 | −0.20441238 | 1 |
| ENSG00000214929 | 0.231067225 | 1 | −0.18457317 | 0.972105264 | 0.513786884 | 1 |
| ENSG00000215405 | −1.317257253 | 1 | 5.455206486 | 0.635517964 | 0.730636605 | 1 |
| ENSG00000215864 | −0.603302235 | 1 | 0.759065108 | 0.832879845 | −0.266125165 | 1 |
| ENSG00000215895 | 0.169580662 | 1 | 2.642918942 | 0.628966264 | 0.087726662 | 1 |
| ENSG00000217094 | 0.108169781 | 1 | −0.084126883 | 0.969781315 | 0.32782765 | 1 |
| ENSG00000218586 | 0.048153136 | 1 | 0.377617976 | 0.92689302 | 0.396082982 | 1 |
| ENSG00000218672 | 0.360545172 | 1 | 4.503237831 | 0.637371741 | 0.128436775 | 1 |
| ENSG00000218823 | −0.149229628 | 1 | 0.370678195 | 0.938341531 | −0.058926155 | 1 |
| ENSG00000221826 | −0.905913781 | 1 | 0.468930195 | 0.922861341 | −0.168206699 | 1 |
| ENSG00000221878 | −0.33451417 | 1 | 0.794876697 | 0.875275045 | −0.515392725 | 1 |
| ENSG00000223414 | 0.396876949 | 1 | 2.170444123 | 0.747425557 | 0.340358512 | 1 |
| ENSG00000223566 | −0.083359397 | 1 | 1.255469251 | 0.773820479 | 0.165805354 | 1 |
| ENSG00000223949 | 0.176177264 | 1 | 0.363654551 | 0.944109028 | 0.785033547 | 1 |
| ENSG00000224059 | 0.085744611 | 1 | 1.119588857 | 0.815513218 | −0.287957535 | 1 |
| ENSG00000224209 | −0.329952707 | 1 | 4.996698997 | 0.63590801 | 1.016744481 | 1 |
| ENSG00000224271 | 0.307895578 | 1 | 0.668981391 | 0.905148092 | 0.475804817 | 1 |
| ENSG00000224367 | −0.312440554 | 1 | −0.119114356 | 0.990683948 | 0.288576794 | 1 |
| ENSG00000224435 | −0.465472953 | 1 | 2.806924373 | 0.701712199 | 0.467138001 | 1 |
| ENSG00000224668 | 0.021954815 | 1 | 2.071297623 | 0.688182163 | 0.145327081 | 1 |
| ENSG00000224743 | −0.442966632 | 1 | 5.789038398 | 0.627668963 | 0.154270252 | 1 |
| ENSG00000225637 | −0.627749272 | 1 | 1.545301662 | 0.794524132 | −0.165724697 | 1 |
| ENSG00000225649 | −0.296958899 | 1 | 2.053521976 | 0.74015481 | 0.717587026 | 1 |
| ENSG00000225813 | −0.127561246 | 1 | 3.110429709 | 0.638925887 | −0.014909586 | 1 |
| ENSG00000225953 | −0.1107115886 | 1 | −0.105927318 | 0.990101886 | 0.110820165 | 1 |
| ENSG00000226057 | −0.06126026 | 1 | −0.683305773 | 0.794524132 | 0.065690247 | 1 |
| ENSG00000226068 | −0.686854837 | 1 | 0.765075801 | 0.86439314 | 0.359560736 | 1 |
| ENSG00000226440 | −0.357778931 | 1 | 1.048188407 | 0.839325295 | −0.132057491 | 1 |
| ENSG00000226741 | −0.012695967 | 1 | 5.855412377 | 0.627668963 | 0.158850168 | 1 |
| ENSG00000226790 | −0.396590339 | 1 | 0.30115281 | 0.904741007 | 0.462516358 | 1 |
| ENSG00000227036 | −0.081342392 | 1 | 1.073510076 | 0.756931946 | 0.134099004 | 1 |
| ENSG00000227471 | −0.187879279 | 1 | 0.646340303 | 0.90427395 | −0.166761865 | 1 |
| ENSG00000227525 | 0.176827018 | 1 | 0.14051531 | 0.980905392 | 0.636393422 | 1 |
| ENSG00000227744 | −0.768704195 | 1 | 2.498837138 | 0.730189271 | −0.334768949 | 1 |
| ENSG00000227827 | 0.347455004 | 1 | −0.893238125 | 0.689965785 | 0.037220004 | 1 |
| ENSG00000228549 | −0.066990016 | 1 | 1.550659524 | 0.6635464 | 0.142348654 | 1 |
| ENSG00000228983 | −0.078571477 | 1 | 0.159220954 | 0.989052009 | −0.315269274 | 1 |
| ENSG00000229147 | 0.163672911 | 1 | 4.59874887 | 0.647433395 | 0.03574788 | 1 |
| ENSG00000229240 | −0.463376599 | 1 | 2.389547223 | 0.723566719 | 0.812962187 | 1 |
| ENSG00000229817 | −1.461237915 | 1 | 5.013969101 | 0.627668963 | 0.574333178 | 1 |
| ENSG00000229972 | 0.069756894 | 1 | 4.841144043 | 0.657456325 | −0.005110713 | 1 |
| ENSG00000230102 | 0.080231864 | 1 | 0.129736847 | 0.98819763 | −0.092566507 | 1 |
| ENSG00000230133 | −0.336765938 | 1 | 4.600759813 | 0.671541129 | 0.054222063 | 1 |
| ENSG00000230392 | −0.67554528 | 1 | 1.884314398 | 0.752977145 | −0.466467709 | 1 |
| ENSG00000230552 | −0.477976855 | 1 | 2.786369729 | 0.657456325 | 0.110748725 | 1 |
| ENSG00000230615 | −0.0775818 | 1 | 2.277546357 | 0.701343297 | 0.278838325 | 1 |
| ENSG00000230873 | −0.606177596 | 1 | 1.869643521 | 0.749476591 | −0.089168797 | 1 |
| ENSG00000231131 | −0.717745459 | 1 | 4.703091051 | 0.634030703 | −0.018903925 | 1 |
| ENSG00000231422 | −0.448623675 | 1 | 1.461416413 | 0.750080465 | 0.581997176 | 1 |
| ENSG00000232392 | 0.141052029 | 1 | −0.415515591 | 0.937722117 | 0.304983976 | 1 |
| ENSG00000232667 | −1.180261087 | 1 | 1.003284752 | 0.858968348 | −0.087170074 | 1 |
| ENSG00000232756 | −0.325138065 | 1 | 5.470222099 | 0.638925887 | 0.241855089 | 1 |
| ENSG00000233183 | −0.460286874 | 1 | 1.142684211 | 0.795694369 | −0.057403209 | 1 |
| ENSG00000233395 | −0.306990217 | 1 | 2.164616494 | 0.727990986 | 0.345611188 | 1 |
| ENSG00000233485 | 0.130714248 | 1 | −0.844030822 | 0.823281306 | −0.389027234 | 1 |
| ENSG00000233539 | −0.342174764 | 1 | 0.799006026 | 0.895729958 | −0.143595171 | 1 |
| ENSG00000233973 | −0.518750937 | 1 | 0.16706038 | 0.994504436 | 0.819352419 | 1 |
| ENSG00000233977 | −0.38786505 | 1 | 4.913123959 | 0.627668963 | −0.65503493 | 1 |
| ENSG00000233991 | −0.158589656 | 1 | 0.701789114 | 0.879150351 | −0.066786642 | 1 |
| ENSG00000234130 | −0.116467879 | 1 | −1.060329506 | 0.682043023 | −0.006454574 | 1 |
| ENSG00000234177 | 0.684663308 | 1 | 5.390634183 | 0.627668963 | 0.749376614 | 1 |
| ENSG00000234512 | 1.365371689 | 1 | 0.390273719 | 0.942340092 | 0.42390419 | 1 |
| ENSG00000234537 | −0.239733924 | 1 | 5.219627926 | 0.627668963 | 0.100726121 | 1 |
| ENSG00000234828 | −0.035141074 | 1 | 0.71765966 | 0.886973057 | 0.222391707 | 1 |
| ENSG00000235711 | 0.034112649 | 1 | 0.790336549 | 0.863106509 | 0.604176861 | 1 |
| ENSG00000235881 | 0.129604823 | 1 | 4.342310927 | 0.641365872 | 0.439379883 | 1 |
| ENSG00000236078 | −1.304003166 | 1 | −0.52242853 | 0.898402607 | 0.032241802 | 1 |
| ENSG00000236229 | 0.16006502 | 1 | 0.253042553 | 0.946406426 | −0.406474523 | 1 |
| ENSG00000236253 | −0.231683569 | 1 | −0.237971332 | 0.969012561 | 0.84186377 | 1 |
| ENSG00000236404 | −0.43102737 | 1 | −0.474971558 | 0.913512862 | 0.324321198 | 1 |
| ENSG00000236445 | −0.270798514 | 1 | 5.432857932 | 0.627668963 | 0.644630745 | 1 |
| ENSG00000236824 | 0.067989767 | 1 | 0.34130967 | 0.905148092 | −0.088910462 | 1 |
| ENSG00000237125 | −0.355426825 | 1 | 0.959425342 | 0.83481487 | 0.111462542 | 1 |
| ENSG00000237222 | −0.634179436 | 1 | 5.771289052 | 0.628886787 | −0.121549704 | 1 |
| ENSG00000237250 | −0.129601041 | 1 | 0.190846578 | 0.981354964 | 0.160652494 | 1 |
| ENSG00000237289 | −0.294426419 | 1 | 1.787760281 | 0.752827393 | 0.548281539 | 1 |
| ENSG00000237390 | −0.081316173 | 1 | 5.343520356 | 0.627668963 | 0.086106809 | 1 |
| ENSG00000237515 | −0.060156577 | 1 | −0.125175047 | 0.984792321 | −0.034481521 | 1 |
| ENSG00000237636 | −0.65296119 | 1 | 1.235736516 | 0.785004308 | −0.203879113 | 1 |
| ENSG00000238116 | −0.374099681 | 1 | 2.506065249 | 0.627668963 | 1.135768642 | 1 |
| ENSG00000239921 | 0.031017749 | 1 | 5.195146623 | 0.627668963 | 0.324780279 | 1 |
| ENSG00000240021 | 0.083051695 | 1 | −0.457221847 | 0.911399121 | 0.205779598 | 1 |
| ENSG00000240694 | −0.057174748 | 1 | 0.821751444 | 0.842124589 | −0.225122808 | 1 |
| ENSG00000240707 | 0.45768823 | 1 | 1.563066998 | 0.764869401 | −0.195990548 | 1 |
| ENSG00000241158 | −0.020040533 | 1 | 0.908350636 | 0.844324446 | 0.688461522 | 1 |
| ENSG00000242512 | −0.520138982 | 1 | 2.09559138 | 0.718736978 | 0.480753033 | 1 |
| ENSG00000242866 | −0.277882673 | 1 | 0.218329488 | 0.952666024 | 0.474792962 | 1 |
| ENSG00000243130 | 0.2156966 | 1 | 0.420866737 | 0.936406883 | 0.456415159 | 1 |
| ENSG00000244694 | −0.585266938 | 1 | 0.280162262 | 0.957471604 | −0.053184384 | 1 |
| ENSG00000245248 | −0.512445862 | 1 | 2.757269299 | 0.717228729 | −0.309869654 | 1 |
| ENSG00000245651 | −0.043949133 | 1 | 4.637420367 | 0.641365872 | 0.080391919 | 1 |
| ENSG00000246695 | −0.396633621 | 1 | 5.168032019 | 0.631753875 | −0.020381824 | 1 |
| ENSG00000247213 | −0.016315976 | 1 | 0.914811357 | 0.836663776 | −0.04897474 | 1 |
| ENSG00000247699 | −0.168355722 | 1 | 4.403825386 | 0.630714789 | 0.238209542 | 1 |
| ENSG00000248587 | −0.270935153 | 1 | 1.525731524 | 0.778988681 | −0.027239838 | 1 |
| ENSG00000248713 | −0.116925333 | 1 | −0.560389013 | 0.859477376 | 0.271840562 | 1 |
| ENSG00000248746 | 0.564712696 | 1 | −0.464953446 | 0.884053868 | 0.124065725 | 1 |
| ENSG00000248966 | −0.315376116 | 1 | 0.606226987 | 0.80060544 | −0.394400901 | 1 |
| ENSG00000248975 | −0.696722695 | 1 | 0.734788549 | 0.906091083 | 0.544431614 | 1 |
| ENSG00000249119 | −0.628092072 | 1 | 2.695430903 | 0.654655709 | 0.516230804 | 1 |
| ENSG00000249215 | 0.154736731 | 1 | −1.104123217 | 0.627668963 | 0.421347089 | 1 |
| ENSG00000249267 | −0.256125279 | 1 | 6.337647595 | 0.627668963 | −0.491599301 | 1 |
| ENSG00000249341 | −0.880234432 | 1 | 0.98818147 | 0.865396845 | −0.229479865 | 1 |
| ENSG00000249464 | −0.514600069 | 1 | 1.191697515 | 0.800133595 | 0.297158453 | 1 |
| ENSG00000249584 | −0.582748024 | 1 | 0.905397905 | 0.853001139 | 0.662950073 | 1 |
| ENSG00000249618 | −0.722916516 | 1 | 5.290100405 | 0.627668963 | 0.977525841 | 1 |
| ENSG00000250049 | 0.565567149 | 1 | 4.638749643 | 0.649773207 | −0.025657469 | 1 |
| ENSG00000250230 | −0.704990533 | 1 | 4.538960207 | 0.631753875 | 0.181526143 | 1 |
| ENSG00000250420 | 0.006459177 | 1 | 1.165649249 | 0.805758306 | 0.299943939 | 1 |
| ENSG00000250423 | −0.592659414 | 1 | 0.396772621 | 0.930402935 | 0.459665173 | 1 |
| ENSG00000250493 | −0.290397732 | 1 | 1.178346789 | 0.813585817 | 0.221318875 | 1 |
| ENSG00000250519 | −0.521459048 | 1 | 0.714452444 | 0.886928104 | 0.294331525 | 1 |
| ENSG00000250546 | −0.546662826 | 1 | 0.645898296 | 0.908465718 | 0.158608765 | 1 |
| ENSG00000251557 | 0.173767605 | 1 | 4.903073168 | 0.636992081 | 0.380350496 | 1 |
| ENSG00000251596 | −0.876823306 | 1 | 5.588638432 | 0.627668963 | −0.068327841 | 1 |
| ENSG00000251705 | −0.480692883 | 1 | 0.605480991 | 0.905148092 | 0.973651682 | 1 |
| ENSG00000253230 | 0.655376032 | 1 | 5.30777447 | 0.627668963 | 0.495127664 | 1 |
| ENSG00000253288 | −0.798184147 | 1 | 1.761778886 | 0.760843839 | −0.45060209 | 1 |
| ENSG00000253301 | −0.266149016 | 1 | 1.768597236 | 0.746197395 | 0.101795721 | 1 |
| ENSG00000253864 | −0.32564569 | 1 | 1.06881022 | 0.853001139 | −0.140130064 | 1 |
| ENSG00000253871 | −1.212840073 | 1 | 4.899864919 | 0.634030703 | −0.128417963 | 1 |
| ENSG00000253974 | −0.494068938 | 1 | 0.695380138 | 0.905537291 | 0.183664543 | 1 |
| ENSG00000254042 | −0.386727632 | 1 | 1.459720136 | 0.770874694 | 0.278099271 | 1 |
| ENSG00000254101 | 0.591458364 | 1 | 1.424625 | 0.813717114 | 0.605533171 | 1 |
| ENSG00000254561 | −1.139245326 | 1 | 1.50940068 | 0.754933936 | 0.059323753 | 1 |
| ENSG00000256343 | −0.537196458 | 1 | 1.275713507 | 0.828463409 | 0.618599755 | 1 |
| ENSG00000256612 | −0.117590576 | 1 | 0.231156174 | 0.944880397 | −0.02046101 | 1 |
| ENSG00000256616 | 0.345935051 | 1 | 5.003784965 | 0.641365872 | −0.366243561 | 1 |
| ENSG00000257008 | 0.848934248 | 1 | −0.102277109 | 0.991463567 | −0.769158893 | 1 |
| ENSG00000257175 | −0.023022917 | 1 | 0.488729454 | 0.870273599 | −0.049325545 | 1 |
| ENSG00000257576 | −0.498873297 | 1 | 2.343052713 | 0.646773142 | −0.636576554 | 1 |
| ENSG00000257907 | −0.512436099 | 1 | 0.588642968 | 0.88207703 | −0.175387446 | 1 |
| ENSG00000258628 | −0.072301517 | 1 | 2.136749014 | 0.669487849 | −0.047239297 | 1 |
| ENSG00000258679 | −0.525392969 | 1 | 0.183725694 | 0.979491691 | −0.287680838 | 1 |
| ENSG00000259010 | −0.240255224 | 1 | 0.197786861 | 0.956817068 | 0.060147849 | 1 |
| ENSG00000259156 | −0.563040176 | 1 | 0.6762214 | 0.863255934 | 0.753375248 | 1 |
| ENSG00000259176 | −0.530044663 | 1 | 2.106754077 | 0.722808749 | 1.540061541 | 1 |
| ENSG00000259380 | −0.58933832 | 1 | 5.11915559 | 0.627668963 | 0.287497927 | 1 |
| ENSG00000259458 | 0.414725309 | 1 | 0.442861703 | 0.946558275 | 0.559307634 | 1 |
| ENSG00000259790 | −0.210937874 | 1 | −1.346601099 | 0.655801493 | 0.633164315 | 1 |
| ENSG00000259841 | −0.79107658 | 1 | 1.107914278 | 0.836986961 | 0.715989445 | 1 |
| ENSG00000260027 | 0.660153746 | 1 | 0.476035227 | 0.906333972 | −0.099631653 | 1 |
| ENSG00000260072 | −0.789539115 | 1 | 0.863110797 | 0.821508914 | 0.12828000 | 1 |
| ENSG00000260290 | 0.104471538 | 1 | −0.836870858 | 0.675458806 | 0.197375256 | 1 |
| ENSG00000260305 | −1.028600501 | 1 | 4.744023963 | 0.628886787 | 0.698875009 | 1 |
| ENSG00000260411 | −0.128473671 | 1 | 0.041607103 | 1 | −0.112690723 | 1 |
| ENSG00000260759 | −0.192880296 | 1 | 0.94955643 | 0.867746032 | −0.051041077 | 1 |
| ENSG00000261104 | −1.123431613 | 1 | 4.860266739 | 0.657456325 | 0.120349508 | 1 |
| ENSG00000261115 | −0.234381639 | 1 | 1.512747564 | 0.661458936 | 0.462069476 | 1 |
| ENSG00000261177 | −0.061233144 | 1 | 1.137537332 | 0.817387218 | −0.510065734 | 1 |
| ENSG00000261275 | −0.4281369 | 1 | 0.786323262 | 0.890831721 | 0.732924735 | 1 |
| ENSG00000261466 | −0.090787283 | 1 | 4.851984871 | 0.638396538 | −0.094510196 | 1 |
| ENSG00000261623 | −0.095785762 | 1 | 1.080068661 | 0.844324446 | 0.864325115 | 1 |
| ENSG00000261649 | −0.660535388 | 1 | 2.043392484 | 0.73954672 | −0.330000743 | 1 |
| ENSG00000261949 | −0.475251506 | 1 | −0.763663195 | 0.850814909 | 0.089947054 | 1 |
| ENSG00000262691 | 0.252943534 | 1 | 5.081334361 | 0.627668963 | −0.401503894 | 1 |
| ENSG00000263711 | 0.040985491 | 1 | 5.347698255 | 0.639135327 | 0.507100706 | 1 |
| ENSG00000264954 | 0.634210849 | 1 | −1.528455556 | 0.684013324 | −0.345406349 | 1 |
| ENSG00000265041 | 0.15210652 | 1 | 0.273182081 | 0.961671576 | 0.078015834 | 1 |
| ENSG00000266172 | −0.456128633 | 1 | 2.845807132 | 0.667458257 | 0.135393857 | 1 |
| ENSG00000266795 | 0.388194442 | 1 | 2.036976544 | 0.746202308 | −0.11233752 | 1 |
| ENSG00000267324 | −0.179694156 | 1 | −0.10327279 | 0.993188995 | 0.459785761 | 1 |
| ENSG00000268388 | −0.295612418 | 1 | 1.970132857 | 0.724282844 | 0.544203429 | 1 |
| ENSG00000268460 | −0.008495927 | 1 | 0.828130352 | 0.869925678 | 0.024780764 | 1 |
| ENSG00000269332 | 0.138816664 | 1 | 1.031492879 | 0.835394728 | 0.410194088 | 1 |

### REFERENCES

1. Steinman L. Immunology of relapse and remission in multiple sclerosis. Annu Rev Immunol 2014; 32:257-81.
2. Fava A, Petri M. Systemic lupus erythematosus: Diagnosis and clinical management. J Autoimmun 2019; 96:1-13.
3. Braun J, Wei B. Body traffic: ecology, genetics, and immunity in inflammatory bowel disease. Annu Rev Pathol 2007; 2:401-29.
4. Braun J, Baraliakos X, Listing J, et al. Differences in the incidence of flares or new onset of inflammatory bowel diseases in patients with ankylosing spondylitis exposed to therapy with anti-tumor necrosis factor alpha agents. Arthritis Rheum 2007; 57:639-47.
5. Sellam J, Marion-Thore S, Dumont F, et al. Use of whole-blood transcriptomic profiling to highlight several pathophysiologic pathways associated with response to rituximab in patients with rheumatoid arthritis: data from a randomized, controlled, open-label trial. Arthritis Rheumatol 2014; 66:2015-25.
6. Sanayama Y, Ikeda K, Saito Y, et al. Prediction of therapeutic responses to tocilizumab in patients with rheumatoid arthritis: biomarkers identified by analysis of gene expression in peripheral blood mononuclear cells using genome-wide DNA microarray. Arthritis Rheumatol 2014; 66:1421-31.
7. Tanino M, Matoba R, Nakamura S, et al. Prediction of efficacy of anti-TNF biologic agent, infliximab, for rheumatoid arthritis patients using a comprehensive transcriptome analysis of white blood cells. Biochem Biophys Res Commun 2009; 387:261-5.
8. Teixeira V H, Olaso R, Martin-Magniette M L, et al. Transcriptome analysis describing new immunity and defense genes in peripheral blood mononuclear cells of rheumatoid arthritis patients. PLoS One 2009; 4:e6803.
9. Zhang Y J, Ioerger T R, Huttenhower C, et al. Global assessment of genomic regions required for growth in Mycobacterium tuberculosis. PLoS Pathog 2012; 8:e1002946.
10. Aletaha D, Neogi T, Silman A J, et al. 2010 Rheumatoid arthritis classification criteria: an American College of Rheumatology/European League Against Rheumatism collaborative initiative. Arthritis Rheum 2010; 62:2569-81.
11. Aletaha D, Neogi T, Silman A J, et al. 2010 rheumatoid arthritis classification criteria: an American College of Rheumatology/European League Against Rheumatism collaborative initiative. Ann Rheum Dis 2010; 69:1580-8.
12. Pincus T, Swearingen C J, Bergman M, Yazici Y. RAPID3 (Routine Assessment of Patient Index Data 3), a rheumatoid arthritis index without formal joint counts for routine care: proposed severity categories compared to disease activity score and clinical disease activity index categories. J Rheumatol 2008; 35:2136-47.
13. Robison E H, Mondala T S, Williams A R, Head S R, Salomon D R, Kurian S M. Whole genome transcript profiling from fingerstick blood samples: a comparison and feasibility study. BMC Genomics 2009; 10:617.
14. Ritchie M E, Phipson B, Wu D, et al. limma powers differential expression analyses for RNA-sequencing and microarray studies. Nucleic Acids Res 2015; 43:e47.
15. Fischer D S, Theis F J, Yosef N. Impulse model-based differential expression analysis of time course sequencing data. Nucleic Acids Res 2018; 46:e119.
16. Bourgon R, Gentleman R, Huber W. Independent filtering increases detection power for high-throughput experiments. Proc Natl Acad Sci USA 2010; 107:9546-51.
17. Monaco G, Lee B, Xu W, et al. RNA-Seq Signatures Normalized by mRNA Abundance Allow Absolute Deconvolution of Human Immune Cell Types. Cell Rep 2019; 26:1627-40 e7.
18. Newman A M, Steen C B, Liu C L, et al. Determining cell type abundance and expression from bulk tissues with digital cytometry. Nat Biotechnol 2019; 37:773-82.
19. Love M I, Huber W, Anders S. Moderated estimation of fold change and dispersion for RNA-seq data with DESeq2. Genome Biol 2014; 15:550.
20. Zhang F, Wei K, Slowikowski K, et al. Defining inflammatory cell states in rheumatoid arthritis joint synovial tissues by integrating single-cell transcriptomics and mass cytometry. Nat Immunol 2019; 20:928-42.
21. Mizoguchi F, Slowikowski K, Wei K, et al. Functionally distinct disease-associated fibroblast subsets in rheumatoid arthritis. Nat Commun 2018; 9:789.
22. Croft A P, Campos J, Jansen K, et al. Distinct fibroblast subsets drive inflammation and damage in arthritis. Nature 2019; 570:246-51.
23. Lefevre S, Knedla A, Tennie C, et al. Synovial fibroblasts spread rheumatoid arthritis to unaffected joints. Nat Med 2009; 15:1414-20.
24. Lu D R, McDavid A N, Kongpachith S, et al. T Cell-Dependent Affinity Maturation and Innate Immune Pathways Differentially Drive Autoreactive B Cell Responses in Rheumatoid Arthritis. Arthritis Rheumatol 2018; 70:1732-44.
25. Mikuls T R, Payne J B, Yu F, et al. Periodontitis and Porphyromonas gingivalis in patients with rheumatoid arthritis. Arthritis Rheumatol 2014; 66:1090-100.
26. Konig M F, Abusleme L, Reinholdt J, et al. Aggregatibacter actinomycetemcomitans-induced hypercitrullination links periodontal infection to autoimmunity in rheumatoid arthritis. Sci Transl Med 2016; 8:369ra176.
27. Eriksson K, Nise L, Alfredsson L, et al. Seropositivity combined with smoking is associated with increased prevalence of periodontitis in patients with rheumatoid arthritis. Ann Rheum Dis 2018; 77:1236-8.
28. Orange et al. N. Engl. J. Med. 2020: 383: 218-228.

### Example 2

### AC2 and AC3 Genes and PRIME Cell Markers

RNAs and Markers

As detailed above, RNA analysis of fingerstick blood samples from RA patients has identified RNAs suitable as markers of RA flares. A first set of markers and RNAs, denoted AC2, was increased 2 weeks prior to flare. AC2 RNAs were enriched with developmental pathways for naïve B cells and leukocytes. A second set of markers, denoted AC3, was increased the week prior to flare and was then decreased for the duration of the flare. AC3 was enriched for pathways not typical of blood samples, particularly cartilage morphogenesis, endochondral bone growth, extracellular matrix organization. AC3 was enriched with sublining fibroblast genes (CD34+HLADR+DKK3+). The AC2 markers are listed below in Table 7. AC3 gene markers are listed below in Table 8.

TABLE 7
AC2 GENES

| Ensembl | Symbol | Description | Score/AUC |
|---|---|---|---|
| ENSG00000204632 | HLA-G | “major histocompatibility complex, class I, G | 5.77E−05 |
| | | [Source: HGNC Symbol; Acc: HGNC: 4964]” | |
| ENSG00000184792 | OSBP2 | oxysterol binding protein 2 | 6.39E−05 |
| | | [Source: HGNC Symbol; Acc: HGNC: 8504] | |
| ENSG00000198892 | SHISA4 | shisa family member 4 | 0.00012762 |
| | | [Source: HGNC Symbol; Acc: HGNC: 27139] | |
| ENSG00000187017 | ESPN | espin | 0.000131036 |
| | | [Source: HGNC Symbol; Acc: HGNC: 13281] | |
| ENSG00000233762 | | | 0.000323289 |
| ENSG00000175130 | MARCKSL1 | MARCKS like 1 | 0.000358619 |
| | | [Source: HGNC Symbol; Acc: HGNC: 7142] | |
| ENSG00000125534 | PPDPF | pancreatic progenitor cell differentiation and | 0.00037025 |
| | | proliferation factor | |
| | | [Source: HGNC Symbol; Acc: HGNC: 16142] | |
| ENSG00000158856 | DMTN | dematin actin binding protein | 0.000376535 |
| | | [Source: HGNC Symbol; Acc: HGNC: 3382] | |
| ENSG00000121413 | ZSCAN18 | zinc finger and SCAN domain containing 18 | 0.000443873 |
| | | [Source: HGNC Symbol; Acc: HGNC: 21037] | |
| ENSG00000230715 | | | 0.000528797 |
| ENSG00000215030 | RPL13P12 | ribosomal protein L13 pseudogene 12 | 0.000588454 |
| | | [Source: HGNC Symbol; Acc: HGNC: 35701] | |
| ENSG00000146540 | C7orf50 | chromosome 7 open reading frame 50 | 0.000639272 |
| | | [Source: HGNC Symbol; Acc: HGNC: 22421] | |
| ENSG00000029534 | ANK1 | ankyrin 1 | 0.000697583 |
| | | [Source: HGNC Symbol; Acc: HGNC: 492] | |
| ENSG00000121104 | FAM117A | family with sequence similarity 117 member A | 0.000697583 |
| | | [Source: HGNC Symbol; Acc: HGNC: 24179] | |
| ENSG00000260231 | JHDM1D-AS1 | JHDM1D antisense RNA 1 (head to head) | 0.00070319 |
| | | [Source: HGNC Symbol; Acc: HGNC: 48959] | |
| ENSG00000211895 | IGHA1 | immunoglobulin heavy constant alpha 1 | 0.000707226 |
| | | [Source: HGNC Symbol; Acc: HGNC: 5478] | |
| ENSG00000173581 | CCDC106 | coiled-coil domain containing 106 | 0.000730947 |
| | | [Source: HGNC Symbol; Acc: HGNC: 30181] | |
| ENSG00000008441 | NFIX | nuclear factor I X | 0.000837162 |
| | | [Source: HGNC Symbol; Acc: HGNC: 7788] | |
| ENSG00000105701 | FKBP8 | FK506 binding protein 8 | 0.000994972 |
| | | [Source: HGNC Symbol; Acc: HGNC: 3724] | |
| ENSG00000079308 | TNS1 | tensin 1 | 0.00113151 |
| | | [Source: HGNC Symbol; Acc: HGNC: 11973] | |
| ENSG00000264063 | MIR3687-2 | microRNA 3687-2 | 0.001218296 |
| | | [Source: HGNC Symbol; Acc: HGNC: 50835] | |
| ENSG00000049089 | COL9A2 | collagen type IX alpha 2 chain | 0.001236713 |
| | | [Source: HGNC Symbol; Acc: HGNC: 2218] | |
| ENSG00000126461 | SCAF1 | SR-related CTD associated factor 1 | 0.00126185 |
| | | [Source: HGNC Symbol; Acc: HGNC: 30403] | |
| ENSG00000243679 | | | 0.001292673 |
| ENSG00000169136 | ATF5 | activating transcription factor 5 | 0.001421682 |
| | | [Source: HGNC Symbol; Acc: HGNC: 790] | |
| ENSG00000181588 | MEX3D | mex-3 RNA binding family member D | 0.001421682 |
| | | [Source: HGNC Symbol; Acc: HGNC: 16734] | |
| ENSG00000103257 | SLC7A5 | solute carrier family 7 member 5 | 0.001574737 |
| | | [Source: HGNC Symbol; Acc: HGNC: 11063] | |
| ENSG00000175931 | UBE2O | ubiquitin conjugating enzyme E2 O | 0.001669619 |
| | | [Source: HGNC Symbol; Acc: HGNC: 29554] | |
| ENSG00000065268 | WDR18 | WD repeat domain 18 | 0.001764407 |
| | | [Source: HGNC Symbol; Acc: HGNC: 17956] | |
| ENSG00000130300 | PLVAP | plasmalemma vesicle associated protein | 0.001779673 |
| | | [Source: HGNC Symbol; Acc: HGNC: 13635] | |
| ENSG00000232434 | AJM1 | apical junction component 1 homolog | 0.001937905 |
| | | [Source: HGNC Symbol; Acc: HGNC: 37284] | |
| ENSG00000197256 | KANK2 | KN motif and ankyrin repeat domains 2 | 0.001945859 |
| | | [Source: HGNC Symbol; Acc: HGNC: 29300] | |
| ENSG00000229809 | ZNF688 | zinc finger protein 688 | 0.002041059 |
| | | [Source: HGNC Symbol; Acc: HGNC: 30489] | |
| ENSG00000130433 | CACNG6 | calcium voltage-gated channel auxiliary subunit | 0.002327494 |
| | | gamma 6 | |
| | | [Source: HGNC Symbol; Acc: HGNC: 13625] | |
| ENSG00000126254 | RBM42 | RNA binding motif protein 42 | 0.002342133 |
| | | [Source: HGNC Symbol; Acc: HGNC: 28117] | |
| ENSG00000013306 | SLC25A39 | solute carrier family 25 member 39 | 0.002354141 |
| | | [Source: HGNC Symbol; Acc: HGNC: 24279] | |
| ENSG00000179837 | NA | NA | 0.002482976 |
| ENSG00000265714 | NA | NA | 0.002482976 |
| ENSG00000172460 | PRSS30P | “serine protease 30, pseudogene | 0.002490713 |
| | | [Source: HGNC Symbol; Acc: HGNC: 28753]” | |
| ENSG00000104983 | CCDC61 | coiled-coil domain containing 61 | 0.002565172 |
| | | [Source: HGNC Symbol; Acc: HGNC: 33629] | |
| ENSG00000211898 | IGHD | immunoglobulin heavy constant delta | 0.002565172 |
| | | [Source: HGNC Symbol; Acc: HGNC: 5480] | |
| ENSG00000055118 | KCNH2 | potassium voltage-gated channel subfamily H | 0.002637584 |
| | | member 2 | |
| | | [Source: HGNC Symbol; Acc: HGNC: 6251] | |
| ENSG00000260335 | | | 0.002639596 |
| ENSG00000104903 | LYL1 | “LYL1, basic helix-loop-helix family member | 0.002696079 |
| | | [Source: HGNC Symbol; Acc: HGNC: 6734]” | |
| ENSG00000099958 | DERL3 | derlin 3 | 0.002709927 |
| | | [Source: HGNC Symbol; Acc: HGNC: 14236] | |
| ENSG00000179526 | SHARPIN | SHANK associated RH domain interactor | 0.002709927 |
| | | [Source: HGNC Symbol; Acc: HGNC: 25321] | |
| ENSG00000133069 | TMCC2 | transmembrane and coiled-coil domain family 2 | 0.002940811 |
| | | [Source: HGNC Symbol; Acc: HGNC: 24239] | |
| ENSG00000240342 | RPS2P5 | ribosomal protein S2 pseudogene 5 | 0.002940811 |
| | | [Source: HGNC Symbol; Acc: HGNC: 31386] | |
| ENSG00000264462 | MIR3648-2 | microRNA 3648-2 | 0.003313878 |
| | | [Source: HGNC Symbol; Acc: HGNC: 50843] | |
| ENSG00000256576 | LINC02361 | long intergenic non-protein coding RNA 2361 | 0.003383286 |
| | | [Source: HGNC Symbol; Acc: HGNC: 53283] | |
| ENSG00000007968 | E2F2 | E2F transcription factor 2 [ | 0.003400809 |
| | | [Source: HGNC Symbol; Acc: HGNC: 3114] | |
| ENSG00000141858 | SAMD1 | sterile alpha motif domain containing 1 | 0.003623721 |
| | | [Source: HGNC Symbol; Acc: HGNC: 17958] | |
| ENSG00000126705 | AHDC1 | AT-hook DNA binding motif containing 1 | 0.004035577 |
| | | [Source: HGNC Symbol; Acc: HGNC: 25230] | |
| ENSG00000141456 | PELP1 | “proline, glutamate and leucine rich protein 1 | 0.00422876 |
| | | [Source: HGNC Symbol; Acc: HGNC: 30134]” | |
| ENSG00000159713 | TPPP3 | tubulin polymerization promoting protein family | 0.004525344 |
| | | member 3 | |
| | | [Source: HGNC Symbol; Acc: HGNC: 24162] | |
| ENSG00000104897 | SF3A2 | splicing factor 3a subunit 2 | 0.004539725 |
| | | [Source: HGNC Symbol; Acc: HGNC: 10766] | |
| ENSG00000063245 | EPN1 | epsin 1 | 0.004540184 |
| | | [Source: HGNC Symbol; Acc: HGNC: 21604] | |
| ENSG00000162783 | IER5 | immediate early response 5 | 0.004549021 |
| | | [Source: HGNC Symbol; Acc: HGNC: 5393] | |
| ENSG00000141582 | CBX4 | chromobox 4 | 0.004686524 |
| | | [Source: HGNC Symbol; Acc: HGNC: 1554] | |
| ENSG00000168159 | RNF187 | ring finger protein 187 | 0.004686524 |
| | | [Source: HGNC Symbol; Acc: HGNC: 27146] | |
| ENSG00000136826 | KLF4 | Kruppel like factor 4 | 0.004891089 |
| | | [Source: HGNC Symbol; Acc: HGNC: 6348] | |
| ENSG00000237214 | | | 0.004891089 |
| ENSG00000066336 | SPI1 | Spi-1 proto-oncogene | 0.005082358 |
| | | [Source: HGNC Symbol; Acc: HGNC: 11241] | |
| ENSG00000172270 | BSG | basigin (Ok blood group) | 0.005082358 |
| | | [Source: HGNC Symbol; Acc: HGNC: 1116] | |
| ENSG00000173868 | PHOSPHO1 | phosphoethanolamine/phosphocholine phosphatase | 0.005082358 |
| | | [Source: HGNC Symbol; Acc: HGNC: 16815] | |
| ENSG00000167182 | SP2 | Sp2 transcription factor | 0.005118791 |
| | | [Source: HGNC Symbol; Acc: HGNC: 11207] | |
| ENSG00000104805 | NUCB1 | nucleobindin 1 | 0.005119064 |
| | | [Source: HGNC Symbol; Acc: HGNC: 8043] | |
| ENSG00000099381 | SETD1A | SET domain containing 1A | 0.00514547 |
| | | [Source: HGNC Symbol; Acc: HGNC: 29010] | |
| ENSG00000185340 | GAS2L1 | growth arrest specific 2 like 1 | 0.00514547 |
| | | [Source: HGNC Symbol; Acc: HGNC: 16955] | |
| ENSG00000007541 | PIGQ | phosphatidylinositol glycan anchor biosynthesis | 0.005166641 |
| | | class Q | |
| | | [Source: HGNC Symbol; Acc: HGNC: 14135] | |
| ENSG00000105610 | KLF1 | Kruppel like factor 1 | 0.005217627 |
| | | [Source: HGNC Symbol; Acc: HGNC: 6345] | |
| ENSG00000137193 | PIM1 | “Pim-1 proto-oncogene, serine/threonine kinase | 0.005247531 |
| | | [Source: HGNC Symbol; Acc: HGNC: 8986]” | |
| ENSG00000171552 | BCL2L1 | BCL2 like 1 | 0.005530506 |
| | | [Source: HGNC Symbol; Acc: HGNC: 992] | |
| ENSG00000172889 | EGFL7 | EGF like domain multiple 7 | 0.005530506 |
| | | [Source: HGNC Symbol; Acc: HGNC: 20594] | |
| ENSG00000213402 | PTPRCAP | “protein tyrosine phosphatase, receptor type C | 0.00560032 |
| | | associated protein | |
| | | [Source: HGNC Symbol; Acc: HGNC: 9667]” | |
| ENSG00000099330 | OCEL1 | occludin/ELL domain containing 1 | 0.005671418 |
| | | [Source: HGNC Symbol; Acc: HGNC: 26221] | |
| ENSG00000147443 | DOK2 | docking protein 2 | 0.005857886 |
| | | [Source: HGNC Symbol; Acc: HGNC: 2991] | |
| ENSG00000182240 | BACE2 | beta-site APP-cleaving enzyme 2 | 0.005857886 |
| | | [Source: HGNC Symbol; Acc: HGNC: 934] | |
| ENSG00000170128 | GPR25 | G protein-coupled receptor 25 | 0.006101348 |
| | | [Source: HGNC Symbol; Acc: HGNC: 4480] | |
| ENSG00000140406 | TLNRD1 | talin rod domain containing 1 | 0.0061541 |
| | | [Source: HGNC Symbol; Acc: HGNC: 13519] | |
| ENSG00000117394 | SLC2A1 | solute carrier family 2 member 1 | 0.006158727 |
| | | [Source: HGNC Symbol; Acc: HGNC: 11005] | |
| ENSG00000141854 | MISP3 | MISP family member 3 | 0.006204755 |
| | | [Source: HGNC Symbol; Acc: HGNC: 26963] | |
| ENSG00000129757 | CDKN1C | cyclin dependent kinase inhibitor 1C | 0.006380434 |
| | | [Source: HGNC Symbol; Acc: HGNC: 1786] | |
| ENSG00000186891 | TNFRSF18 | TNF receptor superfamily member 18 | 0.006421961 |
| | | [Source: HGNC Symbol; Acc: HGNC: 11914] | |
| ENSG00000184897 | H1FX | H1 histone family member X | 0.006465512 |
| | | [Source: HGNC Symbol; Acc: HGNC: 4722] | |
| ENSG00000185236 | RAB11B | “RAB11B, member RAS oncogene family | 0.006602734 |
| | | [Source: HGNC Symbol; Acc: HGNC: 9761]” | |
| ENSG00000030582 | GRN | granulin precursor | 0.006751675 |
| | | [Source: HGNC Symbol; Acc: HGNC: 4601] | |
| ENSG00000071564 | TCF3 | transcription factor 3 | 0.006772165 |
| | | [Source: HGNC Symbol; Acc: HGNC: 11633] | |
| ENSG00000267749 | | | 0.006848491 |
| ENSG00000105373 | NOP53 | NOP53 ribosome biogenesis factor | 0.006952696 |
| | | [Source: HGNC Symbol; Acc: HGNC: 4333] | |
| ENSG00000240445 | FOXO3B | forkhead box O3B pseudogene | 0.006952696 |
| | | [Source: HGNC Symbol; Acc: HGNC: 3822] | |
| ENSG00000127528 | KLF2 | Kruppel like factor 2 | 0.00698206 |
| | | [Source: HGNC Symbol; Acc: HGNC: 6347] | |
| ENSG00000254858 | MPV17L2 | MPV17 mitochondrial inner membrane protein | 0.006997113 |
| | | like 2 | |
| | | [Source: HGNC Symbol; Acc: HGNC: 28177] | |
| ENSG00000130595 | TNNT3 | “troponin T3, fast skeletal type | 0.007133196 |
| | | [Source: HGNC Symbol; Acc: HGNC: 11950]” | |
| ENSG00000130749 | ZC3H4 | zinc finger CCCH-type containing 4 | 0.007141116 |
| | | [Source: HGNC Symbol; Acc: HGNC: 17808] | |
| ENSG00000132819 | RBM38 | RNA binding motif protein 38 | 0.007141116 |
| | | [Source: HGNC Symbol; Acc: HGNC: 15818] | |
| ENSG00000135925 | WNT10A | Wnt family member 10A | 0.007141116 |
| | | [Source: HGNC Symbol; Acc: HGNC: 13829] | |
| ENSG00000205639 | MFSD2B | major facilitator superfamily domain containing 2B | 0.007141116 |
| | | [Source: HGNC Symbol; Acc: HGNC: 37207] | |
| ENSG00000213763 | ACTBP2 | “actin, beta pseudogene 2 | 0.007141116 |
| | | [Source: HGNC Symbol; Acc: HGNC: 135]” | |
| ENSG00000261221 | ZNF865 | zinc finger protein 865 | 0.007141116 |
| | | [Source: HGNC Symbol; Acc: HGNC: 38705] | |
| ENSG00000171611 | PTCRA | pre T cell antigen receptor alpha | 0.007255358 |
| | | [Source: HGNC Symbol; Acc: HGNC: 21290] | |
| ENSG00000161642 | ZNF385A | zinc finger protein 385A | 0.007304748 |
| | | [Source: HGNC Symbol; Acc: HGNC: 17521] | |
| ENSG00000226608 | FTLP3 | ferritin light chain pseudogene 3 | 0.007304748 |
| | | [Source: HGNC Symbol; Acc: HGNC: 4000] | |
| ENSG00000170684 | ZNF296 | zinc finger protein 296 | 0.007327997 |
| | | [Source: HGNC Symbol; Acc: HGNC: 15981] | |
| ENSG00000213638 | ADAT3 | “adenosine deaminase, tRNA specific 3 | 0.007343983 |
| | | [Source: HGNC Symbol; Acc: HGNC: 25151]” | |
| ENSG00000179262 | RAD23A | “RAD23 homolog A, nucleotide excision repair protein | 0.0073448 |
| | | [Source: HGNC Symbol; Acc: HGNC: 9812]” | |
| ENSG00000196126 | HLA-DRB1 | “major histocompatibility complex, class II, DR beta 1 | 0.00745996 |
| | | [Source: HGNC Symbol; Acc: HGNC: 4948]” | |
| ENSG00000197149 | | | 0.007535198 |
| ENSG00000213820 | RPL13P2 | ribosomal protein L13 pseudogene 2 | 0.007619923 |
| | | [Source: HGNC Symbol; Acc: HGNC: 16342] | |
| ENSG00000225331 | LINC01678 | long intergenic non-protein coding RNA 1678 | 0.007619923 |
| | | [Source: HGNC Symbol; Acc: HGNC: 52466] | |
| ENSG00000235605 | | | 0.007619923 |
| ENSG00000183092 | BEGAIN | brain enriched guanylate kinase associated | 0.007755013 |
| | | [Source: HGNC Symbol; Acc: HGNC: 24163] | |
| ENSG00000105369 | CD79A | CD79a molecule | 0.007835589 |
| | | [Source: HGNC Symbol; Acc: HGNC: 1698] | |
| ENSG00000160256 | FAM207A | family with sequence similarity 207 member A | 0.007901726 |
| | | [Source: HGNC Symbol; Acc: HGNC: 15811] | |
| ENSG00000105516 | DBP | D-box binding PAR bZIP transcription factor | 0.00795596 |
| | | [Source: HGNC Symbol; Acc: HGNC: 2697] | |
| ENSG00000179094 | PER1 | period circadian regulator 1 | 0.008010463 |
| | | [Source: HGNC Symbol; Acc: HGNC: 8845] | |
| ENSG00000154146 | NRGN | neurogranin | 0.008019602 |
| | | [Source: HGNC Symbol; Acc: HGNC: 8000] | |
| ENSG00000160813 | PPP1R35 | protein phosphatase 1 regulatory subunit 35 | 0.008019602 |
| | | [Source: HGNC Symbol; Acc: HGNC: 28320] | |
| ENSG00000152082 | MZT2B | mitotic spindle organizing protein 2B | 0.008149414 |
| | | [Source: HGNC Symbol; Acc: HGNC: 25886] | |
| ENSG00000115274 | INO80B | INO80 complex subunit B | 0.008388785 |
| | | [Source: HGNC Symbol; Acc: HGNC: 13324] | |
| ENSG00000185112 | FAM43A | family with sequence similarity 43 member A | 0.008409751 |
| | | [Source: HGNC Symbol; Acc: HGNC: 26888] | |
| ENSG00000130592 | LSP1 | lymphocyte-specific protein 1 | 0.00866593 |
| | | [Source: HGNC Symbol; Acc: HGNC: 6707] | |
| ENSG00000077348 | EXOSC5 | exosome component 5 | 0.008679008 |
| | | [Source: HGNC Symbol; Acc: HGNC: 24662] | |
| ENSG00000196498 | NCOR2 | nuclear receptor corepressor 2 | 0.008729326 |
| | | [Source: HGNC Symbol; Acc: HGNC: 7673] | |
| ENSG00000132382 | MYBBP1A | MYB binding protein 1a | 0.008886691 |
| | | [Source: HGNC Symbol; Acc: HGNC: 7546] | |
| ENSG00000104885 | DOT1L | DOT1 like histone lysine methyltransferase | 0.008912378 |
| | | [Source: HGNC Symbol; Acc: HGNC: 24948] | |
| ENSG00000153443 | UBALD1 | UBA like domain containing 1 | 0.008912378 |
| | | [Source: HGNC Symbol; Acc: HGNC: 29576] | |
| ENSG00000070182 | SPTB | “spectrin beta, erythrocytic | 0.008927471 |
| | | [Source: HGNC Symbol; Acc: HGNC: 11274]” | |
| ENSG00000168517 | HEXIM2 | hexamethylene bisacetamide inducible 2 | 0.008959412 |
| | | [Source: HGNC Symbol; Acc: HGNC: 28591] | |
| ENSG00000090674 | MCOLN1 | mucolipin 1 | 0.009327518 |
| | | [Source: HGNC Symbol; Acc: HGNC: 13356] | |
| ENSG00000198816 | ZNF358 | zinc finger protein 358 | 0.009506451 |
| | | [Source: HGNC Symbol; Acc: HGNC: 16838] | |
| ENSG00000175334 | BANF1 | barrier to autointegration factor 1 | 0.009647997 |
| | | [Source: HGNC Symbol; Acc: HGNC: 17397] | |
| ENSG00000125520 | SLC2A4RG | SLC2A4 regulator | 0.009686596 |
| | | [Source: HGNC Symbol; Acc: HGNC: 15930] | |
| ENSG00000141084 | RANBP10 | RAN binding protein 10 | 0.009715508 |
| | | [Source: HGNC Symbol; Acc: HGNC: 29285] | |
| ENSG00000149016 | TUT1 | “terminal uridylyl transferase 1, U6 snRNA-specific | 0.009768686 |
| | | [Source: HGNC Symbol; Acc: HGNC: 26184]” | |
| ENSG00000178951 | ZBTB7A | zinc finger and BTB domain containing 7A | 0.009810065 |
| | | [Source: HGNC Symbol; Acc: HGNC: 18078] | |
| ENSG00000186111 | PIP5K1C | phosphatidylinositol-4-phosphate 5-kinase type 1 gamma | 0.009810065 |
| | | [Source: HGNC Symbol; Acc: HGNC: 8996] | |
| ENSG00000184481 | FOXO4 | forkhead box O4 | 0.009820284 |
| | | [Source: HGNC Symbol; Acc: HGNC: 7139] | |
| ENSG00000064961 | HMG20B | high mobility group 20B | 0.009858965 |
| | | [Source: HGNC Symbol; Acc: HGNC: 5002] | |
| ENSG00000108309 | RUNDC3A | RUN domain containing 3A | 0.010055443 |
| | | [Source: HGNC Symbol; Acc: HGNC: 16984] | |
| ENSG00000130165 | ELOF1 | elongation factor 1 homolog | 0.010212535 |
| | | [Source: HGNC Symbol; Acc: HGNC: 28691] | |
| ENSG00000130159 | ECSIT | ECSIT signalling integrator | 0.010245658 |
| | | [Source: HGNC Symbol; Acc: HGNC: 29548] | |
| ENSG00000244560 | | | 0.010245658 |
| ENSG00000125148 | MT2A | metallothionein 2A | 0.01061889 |
| | | [Source: HGNC Symbol; Acc: HGNC: 7406] | |
| ENSG00000131116 | ZNF428 | zinc finger protein 428 | 0.010712491 |
| | | [Source: HGNC Symbol; Acc: HGNC: 20804] | |
| ENSG00000105617 | LENG1 | leukocyte receptor cluster member 1 | 0.010999325 |
| | | [Source: HGNC Symbol; Acc: HGNC: 15502] | |
| ENSG00000139718 | SETD1B | SET domain containing 1B | 0.011038344 |
| | | [Source: HGNC Symbol; Acc: HGNC: 29187] | |
| ENSG00000106665 | CLIP2 | CAP-Gly domain containing linker protein 2 | 0.011064297 |
| | | [Source: HGNC Symbol; Acc: HGNC: 2586] | |
| ENSG00000130821 | SLC6A8 | solute carrier family 6 member 8 | 0.011213055 |
| | | [Source: HGNC Symbol; Acc: HGNC: 11055] | |
| ENSG00000184232 | OAF | out at first homolog | 0.011286635 |
| | | [Source: HGNC Symbol; Acc: HGNC: 28752] | |
| ENSG00000179820 | MYADM | myeloid associated differentiation marker | 0.011330192 |
| | | [Source: HGNC Symbol; Acc: HGNC: 7544] | |
| ENSG00000127580 | WDR24 | WD repeat domain 24 | 0.011570001 |
| | | [Source: HGNC Symbol; Acc: HGNC: 20852] | |
| ENSG00000004939 | SLC4A1 | solute carrier family 4 member 1 (Diego blood group) | 0.011732314 |
| | | [Source: HGNC Symbol; Acc: HGNC: 11027] | |
| ENSG00000130522 | JUND | “JunD proto-oncogene, AP-1 transcription factor | 0.011813745 |
| | | subunit | |
| | | [Source: HGNC Symbol; Acc: HGNC: 6206]” | |
| ENSG00000148362 | PAXX | “PAXX, non-homologous end joining factor | 0.011821074 |
| | | [Source: HGNC Symbol; Acc: HGNC: 27849]” | |
| ENSG00000262902 | MTCO1P40 | mitochondrially encoded cytochrome c oxidase | 0.011821074 |
| | | I pseudogene 40 | |
| | | [Source: HGNC Symbol; Acc: HGNC: 52105] | |
| ENSG00000167671 | UBXN6 | UBX domain protein 6 | 0.011831088 |
| | | [Source: HGNC Symbol; Acc: HGNC: 14928] | |
| ENSG00000125457 | MIF4GD | MIF4G domain containing | 0.011851589 |
| | | [Source: HGNC Symbol; Acc: HGNC: 24030] | |
| ENSG00000146066 | HIGD2A | HIG1 hypoxia inducible domain family member 2A | 0.011914767 |
| | | [Source: HGNC Symbol; Acc: HGNC: 28311] | |
| ENSG00000184221 | OLIG1 | oligodendrocyte transcription factor 1 | 0.011914767 |
| | | [Source: HGNC Symbol; Acc: HGNC: 16983] | |
| ENSG00000260316 | | | 0.0119947 |
| ENSG00000124762 | CDKN1A | cyclin dependent kinase inhibitor 1A | 0.012077375 |
| | | [Source: HGNC Symbol; Acc: HGNC: 1784] | |
| ENSG00000103148 | NPRL3 | “NPR3 like, GATOR1 complex subunit | 0.012137266 |
| | | [Source: HGNC Symbol; Acc: HGNC: 14124]” | |
| ENSG00000179115 | FARSA | phenylalanyl-tRNA synthetase alpha subunit | 0.012137266 |
| | | [Source: HGNC Symbol; Acc: HGNC: 3592] | |
| ENSG00000120896 | SORBS3 | sorbin and SH3 domain containing 3 | 0.012150664 |
| | | [Source: HGNC Symbol; Acc: HGNC: 30907] | |
| ENSG00000174886 | NDUFA11 | NADH: ubiquinone oxidoreductase subunit A11 | 0.012268883 |
| | | [Source: HGNC Symbol; Acc: HGNC: 20371] | |
| ENSG00000102145 | GATA1 | GATA binding protein 1 | 0.012285964 |
| | | [Source: HGNC Symbol; Acc: HGNC: 4170] | |
| ENSG00000166428 | PLD4 | phospholipase D family member 4 | 0.012401569 |
| | | [Source: HGNC Symbol; Acc: HGNC: 23792] | |
| ENSG00000213015 | ZNF580 | zinc finger protein 580 | 0.012630003 |
| | | [Source: HGNC Symbol; Acc: HGNC: 29473] | |
| ENSG00000142544 | CTU1 | cytosolic thiouridylase subunit 1 | 0.012676606 |
| | | [Source: HGNC Symbol; Acc: HGNC: 29590] | |
| ENSG00000085644 | ZNF213 | zinc finger protein 213 | 0.012935817 |
| | | [Source: HGNC Symbol; Acc: HGNC: 13005] | |
| ENSG00000003249 | DBNDD1 | dysbindin domain containing 1 | 0.013109402 |
| | | [Source: HGNC Symbol; Acc: HGNC: 28455] | |
| ENSG00000221288 | MIR663B | microRNA 663b | 0.013333238 |
| | | [Source: HGNC Symbol; Acc: HGNC: 35270] | |
| ENSG00000042062 | RIPOR3 | RIPOR family member 3 | 0.013356858 |
| | | [Source: HGNC Symbol; Acc: HGNC: 16168] | |
| ENSG00000105329 | TGFB1 | transforming growth factor beta 1 | 0.013356858 |
| | | [Source: HGNC Symbol; Acc: HGNC: 11766] | |
| ENSG00000116871 | MAP7D1 | MAP7 domain containing 1 | 0.013356858 |
| | | [Source: HGNC Symbol; Acc: HGNC: 25514] | |
| ENSG00000168298 | HIST1H1E | histone cluster 1 H1 family member e | 0.013400191 |
| | | [Source: HGNC Symbol; Acc: HGNC: 4718] | |
| ENSG00000127666 | TICAM1 | toll like receptor adaptor molecule 1 | 0.013447765 |
| | | [Source: HGNC Symbol; Acc: HGNC: 18348] | |
| ENSG00000166886 | NAB2 | NGFI-A binding protein 2 | 0.013572419 |
| | | [Source: HGNC Symbol; Acc: HGNC: 7627] | |
| ENSG00000112787 | FBRSL1 | fibrosin like 1 | 0.013760569 |
| | | [Source: HGNC Symbol; Acc: HGNC: 29308] | |
| ENSG00000100243 | CYB5R3 | cytochrome b5 reductase 3 | 0.013883197 |
| | | [Source: HGNC Symbol; Acc: HGNC: 2873] | |
| ENSG00000197457 | STMN3 | stathmin 3 | 0.013964273 |
| | | [Source: HGNC Symbol; Acc: HGNC: 15926] | |
| ENSG00000255441 | | | 0.013965194 |
| ENSG00000173801 | JUP | junction plakoglobin | 0.014437619 |
| | | [Source: HGNC Symbol; Acc: HGNC: 6207] | |
| ENSG00000224614 | TNK2-AS1 | TNK2 antisense RNA 1 | 0.014437619 |
| | | [Source: HGNC Symbol; Acc: HGNC: 49093] | |
| ENSG00000058453 | CROCC | “ciliary rootlet coiled-coil, rootletin | 0.014522615 |
| | | [Source: HGNC Symbol; Acc: HGNC: 21299]” | |
| ENSG00000079313 | REXO1 | RNA exonuclease 1 homolog | 0.014579462 |
| | | [Source: HGNC Symbol; Acc: HGNC: 24616] | |
| ENSG00000154102 | C16orf74 | chromosome 16 open reading frame 74 | 0.014732236 |
| | | [Source: HGNC Symbol; Acc: HGNC: 23362] | |
| ENSG00000172650 | AGAP5 | “ArfGAP with GTPase domain, ankyrin repeat | 0.014761715 |
| | | and PH domain 5 | |
| | | [Source: HGNC Symbol; Acc: HGNC: 23467]” | |
| ENSG00000159733 | ZFYVE28 | zinc finger FYVE-type containing 28 | 0.014792652 |
| | | [Source: HGNC Symbol; Acc: HGNC: 29334] | |
| ENSG00000019582 | CD74 | CD74 molecule | 0.014946895 |
| | | [Source: HGNC Symbol; Acc: HGNC: 1697] | |
| ENSG00000211771 | TRBJ2-7 | T cell receptor beta joining 2-7 | 0.014961068 |
| | | [Source: HGNC Symbol; Acc: HGNC: 12175] | |
| ENSG00000214309 | MBLAC1 | metallo-beta-lactamase domain containing 1 | 0.014976932 |
| | | [Source: HGNC Symbol; Acc: HGNC: 22180] | |
| ENSG00000187266 | EPOR | erythropoietin receptor | 0.015342955 |
| | | [Source: HGNC Symbol; Acc: HGNC: 3416] | |
| ENSG00000108106 | UBE2S | ubiquitin conjugating enzyme E2 S | 0.015459542 |
| | | [Source: HGNC Symbol; Acc: HGNC: 17895] | |
| ENSG00000185838 | GNB1L | G protein subunit beta 1 like | 0.015552452 |
| | | [Source: HGNC Symbol; Acc: HGNC: 4397] | |
| ENSG00000228594 | FNDC10 | fibronectin type III domain containing 10 | 0.015552452 |
| | | [Source: HGNC Symbol; Acc: HGNC: 42951] | |
| ENSG00000126464 | PRR12 | proline rich 12 | 0.015622838 |
| | | [Source: HGNC Symbol; Acc: HGNC: 29217] | |
| ENSG00000084092 | NOA1 | nitric oxide associated 1 | 0.015753463 |
| | | [Source: HGNC Symbol; Acc: HGNC: 28473] | |
| ENSG00000105227 | PRX | periaxin | 0.015787169 |
| | | [Source: HGNC Symbol; Acc: HGNC: 13797] | |
| ENSG00000260401 | | | 0.015787169 |
| ENSG00000159840 | ZYX | zyxin | 0.015829354 |
| | | [Source: HGNC Symbol; Acc: HGNC: 13200] | |
| ENSG00000197483 | ZNF628 | zinc finger protein 628 | 0.015834462 |
| | | [Source: HGNC Symbol; Acc: HGNC: 28054] | |
| ENSG00000182572 | NA | NA | 0.015908819 |
| ENSG00000154035 | NA | NA | 0.015942034 |
| ENSG00000161618 | ALDH16A1 | aldehyde dehydrogenase 16 family member A1 | 0.015942034 |
| | | [Source: HGNC Symbol; Acc: HGNC: 28114] | |
| ENSG00000124575 | HIST1H1D | histone cluster 1 H1 family member d | 0.015948868 |
| | | [Source: HGNC Symbol; Acc: HGNC: 4717] | |
| ENSG00000196092 | PAX5 | paired box 5 | 0.01597311 |
| | | [Source: HGNC Symbol; Acc: HGNC: 8619] | |
| ENSG00000105429 | MEGF8 | multiple EGF like domains 8 | 0.015986308 |
| | | [Source: HGNC Symbol; Acc: HGNC: 3233] | |
| ENSG00000213753 | CENPBD1P1 | CENPB DNA-binding domains containing 1 | 0.016008143 |
| | | pseudogene 1 | |
| | | [Source: HGNC Symbol; Acc: HGNC: 28421] | |
| ENSG00000179627 | ZBTB42 | zinc finger and BTB domain containing 42 | 0.016166469 |
| | | [Source: HGNC Symbol; Acc: HGNC: 32550] | |
| ENSG00000107816 | LZTS2 | leucine zipper tumor suppressor 2 | 0.016243979 |
| | | [Source: HGNC Symbol; Acc: HGNC: 29381] | |
| ENSG00000183779 | ZNF703 | zinc finger protein 703 | 0.016243979 |
| | | [Source: HGNC Symbol; Acc: HGNC: 25883] | |
| ENSG00000203950 | RTL8A | retrotransposon Gag like 8A | 0.01630694 |
| | | [Source: HGNC Symbol; Acc: HGNC: 24514] | |
| ENSG00000088826 | SMOX | spermine oxidase | 0.016416472 |
| | | [Source: HGNC Symbol; Acc: HGNC: 15862] | |
| ENSG00000105298 | CACTIN | “cactin, spliceosome C complex subunit | 0.016416472 |
| | | [Source: HGNC Symbol; Acc: HGNC: 29938]” | |
| ENSG00000137218 | FRS3 | fibroblast growth factor receptor substrate 3 | 0.016416472 |
| | | [Source: HGNC Symbol; Acc: HGNC: 16970] | |
| ENSG00000175550 | DRAP1 | DR1 associated protein 1 | 0.016587059 |
| | | [Source: HGNC Symbol; Acc: HGNC: 3019] | |
| ENSG00000166165 | CKB | creatine kinase B | 0.01659925 |
| | | [Source: HGNC Symbol; Acc: HGNC: 1991] | |
| ENSG00000162366 | PDZK1IP1 | PDZK1 interacting protein 1 | 0.016705327 |
| | | [Source: HGNC Symbol; Acc: HGNC: 16887] | |
| ENSG00000184428 | TOP1MT | DNA topoisomerase I mitochondrial | 0.016722415 |
| | | [Source: HGNC Symbol; Acc: HGNC: 29787] | |
| ENSG00000130479 | MAP1S | microtubule associated protein IS | 0.016796937 |
| | | [Source: HGNC Symbol; Acc: HGNC: 15715] | |
| ENSG00000171222 | SCAND1 | SCAN domain containing 1 | 0.016821415 |
| | | [Source: HGNC Symbol; Acc: HGNC: 10566] | |
| ENSG00000171223 | JUNB | “JunB proto-oncogene, AP-1 transcription | 0.016966042 |
| | | factor subunit | |
| | | [Source: HGNC Symbol; Acc: HGNC: 6205]” | |
| ENSG00000107902 | LHPP | phospholysine phosphohistidine inorganic | 0.017052413 |
| | | pyrophosphate phosphatase | |
| | | [Source: HGNC Symbol; Acc: HGNC: 30042] | |
| ENSG00000170271 | FAXDC2 | fatty acid hydroxylase domain containing 2 | 0.017052413 |
| | | [Source: HGNC Symbol; Acc: HGNC: 1334] | |
| ENSG00000100325 | ASCC2 | activating signal cointegrator 1 complex subunit 2 | 0.017132234 |
| | | [Source: HGNC Symbol; Acc: HGNC: 24103] | |
| ENSG00000142694 | EVA1B | eva-1 homolog B | 0.017132234 |
| | | [Source: HGNC Symbol; Acc: HGNC: 25558] | |
| ENSG00000064201 | TSPAN32 | tetraspanin 32 | 0.017210927 |
| | | [Source: HGNC Symbol; Acc: HGNC: 13410] | |
| ENSG00000157911 | PEX10 | peroxisomal biogenesis factor 10 | 0.017211726 |
| | | [Source: HGNC Symbol; Acc: HGNC: 8851] | |
| ENSG00000079432 | CIC | capicua transcriptional repressor | 0.017339051 |
| | | [Source: HGNC Symbol; Acc: HGNC: 14214] | |
| ENSG00000188825 | LINC00910 | long intergenic non-protein coding RNA 910 | 0.017339051 |
| | | [Source: HGNC Symbol; Acc: HGNC: 44361] | |
| ENSG00000196961 | AP2A1 | adaptor related protein complex 2 alpha 1 subunit | 0.017339051 |
| | | [Source: HGNC Symbol; Acc: HGNC: 561] | |
| ENSG00000214279 | SCART1 | scavenger receptor family member expressed on | 0.017339051 |
| | | T cells 1 | |
| | | [Source: HGNC Symbol; Acc: HGNC: 32411] | |
| ENSG00000272449 | | | 0.017339051 |
| ENSG00000104973 | MED25 | mediator complex subunit 25 | 0.017392388 |
| | | [Source: HGNC Symbol; Acc: HGNC: 28845] | |
| ENSG00000180767 | CHST13 | carbohydrate sulfotransferase 13 | 0.017392388 |
| | | [Source: HGNC Symbol; Acc: HGNC: 21755] | |
| ENSG00000227232 | WASH7P | WAS protein family homolog 7 pseudogene | 0.017392388 |
| | | [Source: HGNC Symbol; Acc: HGNC: 38034] | |
| ENSG00000162302 | RPS6KA4 | ribosomal protein S6 kinase A4 | 0.017767827 |
| | | [Source: HGNC Symbol; Acc: HGNC: 10433] | |
| ENSG00000136840 | ST6GALNAC4 | “ST6 N-acetylgalactosaminide | 0.017832974 |
| | | alpha-2,6-sialyltransferase 4 | |
| | | [Source: HGNC Symbol; Acc: HGNC: 17846]” | |
| ENSG00000160404 | TOR2A | torsin family 2 member A | 0.018020454 |
| | | [Source: HGNC Symbol; Acc: HGNC: 11996] | |
| ENSG00000233038 | | | 0.018071818 |
| ENSG00000243449 | C4orf48 | chromosome 4 open reading frame 48 | 0.018116836 |
| | | [Source: HGNC Symbol; Acc: HGNC: 34437] | |
| ENSG00000160050 | CCDC28B | coiled-coil domain containing 28B | 0.01812987 |
| | | [Source: HGNC Symbol; Acc: HGNC: 28163] | |
| ENSG00000138623 | SEMA7A | semaphorin 7A (John Milton Hagen blood group) | 0.01834001 |
| | | [Source: HGNC Symbol; Acc: HGNC: 10741] | |
| ENSG00000101439 | CST3 | cystatin C | 0.018421259 |
| | | [Source: HGNC Symbol; Acc: HGNC: 2475] | |
| ENSG00000100368 | CSF2RB | colony stimulating factor 2 receptor beta | 0.01865112 |
| | | common subunit | |
| | | [Source: HGNC Symbol; Acc: HGNC: 2436] | |
| ENSG00000006015 | REX1BD | required for excision 1-B domain containing | 0.01871477 |
| | | [Source: HGNC Symbol; Acc: HGNC: 26098] | |
| ENSG00000011451 | WIZ | widely interspaced zinc finger motifs | 0.018812736 |
| | | [Source: HGNC Symbol; Acc: HGNC: 30917] | |
| ENSG00000160888 | IER2 | immediate early response 2 | 0.018812736 |
| | | [Source: HGNC Symbol; Acc: HGNC: 28871] | |
| ENSG00000174807 | CD248 | CD248 molecule | 0.018812736 |
| | | [Source: HGNC Symbol; Acc: HGNC: 18219] | |
| ENSG00000099821 | POLRMT | RNA polymerase mitochondrial | 0.018832922 |
| | | [Source: HGNC Symbol; Acc: HGNC: 9200] | |
| ENSG00000211899 | IGHM | immunoglobulin heavy constant mu | 0.018832922 |
| | | [Source: HGNC Symbol; Acc: HGNC: 5541] | |
| ENSG00000130313 | PGLS | 6-phosphogluconolactonase | 0.019015294 |
| | | [Source: HGNC Symbol; Acc: HGNC: 8903] | |
| ENSG00000165702 | GFI1B | growth factor independent 1B transcriptional | 0.019015294 |
| | | repressor | |
| | | [Source: HGNC Symbol; Acc: HGNC: 4238] | |
| ENSG00000196557 | CACNA1H | calcium voltage-gated channel subunit alpha1 H | 0.019108077 |
| | | [Source: HGNC Symbol; Acc: HGNC: 1395] | |
| ENSG00000188486 | H2AFX | H2A histone family member X | 0.019120708 |
| | | [Source: HGNC Symbol; Acc: HGNC: 4739] | |
| ENSG00000103260 | METRN | “meteorin, glial cell differentiation regulator | 0.01915234 |
| | | [Source: HGNC Symbol; Acc: HGNC: 14151]” | |
| ENSG00000166925 | TSC22D4 | TSC22 domain family member 4 | 0.01920241 |
| | | [Source: HGNC Symbol; Acc: HGNC: 21696] | |
| ENSG00000106266 | SNX8 | sorting nexin 8 | 0.019236897 |
| | | [Source: HGNC Symbol; Acc: HGNC: 14972] | |
| ENSG00000110400 | NECTIN1 | nectin cell adhesion molecule 1 | 0.01926502 |
| | | [Source: HGNC Symbol; Acc: HGNC: 9706] | |
| ENSG00000088992 | TESC | tescalcin | 0.019687862 |
| | | [Source: HGNC Symbol; Acc: HGNC: 26065] | |
| ENSG00000126368 | NR1D1 | nuclear receptor subfamily 1 group D member 1 | 0.019740714 |
| | | [Source: HGNC Symbol; Acc: HGNC: 7962] | |
| ENSG00000103202 | NME4 | NME/NM23 nucleoside diphosphate kinase 4 | 0.019829586 |
| | | [Source: HGNC Symbol; Acc: HGNC: 7852] | |
| ENSG00000213626 | LBH | limb bud and heart development | 0.019900152 |
| | | [Source: HGNC Symbol; Acc: HGNC: 29532] | |
| ENSG00000138629 | UBL7 | ubiquitin like 7 | 0.019916102 |
| | | [Source: HGNC Symbol; Acc: HGNC: 28221] | |
| ENSG00000254614 | | | 0.019916102 |
| ENSG00000116521 | SCAMP3 | secretory carrier membrane protein 3 | 0.019953714 |
| | | [Source: HGNC Symbol; Acc: HGNC: 10565] | |
| ENSG00000132481 | TRIM47 | tripartite motif containing 47 | 0.019989295 |
| | | [Source: HGNC Symbol; Acc: HGNC: 19020] | |
| ENSG00000105699 | LSR | lipolysis stimulated lipoprotein receptor | 0.019999965 |
| | | [Source: HGNC Symbol; Acc: HGNC: 29572] | |
| ENSG00000125503 | PPP1R12C | protein phosphatase 1 regulatory subunit 12C | 0.020063533 |
| | | [Source: HGNC Symbol; Acc: HGNC: 14947] | |
| ENSG00000103056 | SMPD3 | sphingomyelin phosphodiesterase 3 | 0.020115844 |
| | | [Source: HGNC Symbol; Acc: HGNC: 14240] | |
| ENSG00000156381 | ANKRD9 | ankyrin repeat domain 9 | 0.020225168 |
| | | [Source: HGNC Symbol; Acc: HGNC: 20096] | |
| ENSG00000197471 | SPN | sialophorin | 0.020225168 |
| | | [Source: HGNC Symbol; Acc: HGNC: 11249] | |
| ENSG00000197471 | SPN | sialophorin | 0.020225168 |
| | | [Source: HGNC Symbol; Acc: HGNC: 11249] | |
| ENSG00000063854 | HAGH | hydroxyacylglutathione hydrolase | 0.020247513 |
| | | [Source: HGNC Symbol; Acc: HGNC: 4805] | |
| ENSG00000130590 | SAMD10 | sterile alpha motif domain containing 10 | 0.020258063 |
| | | [Source: HGNC Symbol; Acc: HGNC: 16129] | |
| ENSG00000167664 | TMIGD2 | transmembrane and immunoglobulin domain | 0.020258063 |
| | | containing 2 | |
| | | [Source: HGNC Symbol; Acc: HGNC: 28324] | |
| ENSG00000146083 | RNF44 | ring finger protein 44 | 0.020327471 |
| | | [Source: HGNC Symbol; Acc: HGNC: 19180] | |
| ENSG00000231925 | TAPBP | TAP binding protein | 0.020387859 |
| | | [Source: HGNC Symbol; Acc: HGNC: 11566] | |
| ENSG00000198858 | R3HDM4 | R3H domain containing 4 | 0.020462672 |
| | | [Source: HGNC Symbol; Acc: HGNC: 28270] | |
| ENSG00000135924 | DNAJB2 | DnaJ heat shock protein family (Hsp40) member B2 | 0.020505106 |
| | | [Source: HGNC Symbol; Acc: HGNC: 5228] | |
| ENSG00000239732 | TLR9 | toll like receptor 9 | 0.02065324 |
| | | [Source: HGNC Symbol; Acc: HGNC: 15633] | |
| ENSG00000115268 | RPS15 | ribosomal protein S15 | 0.020839375 |
| | | [Source: HGNC Symbol; Acc: HGNC: 10388] | |
| ENSG00000108798 | ABI3 | ABI family member 3 | 0.02085252 |
| | | [Source: HGNC Symbol; Acc: HGNC: 29859] | |
| ENSG00000119669 | IRF2BPL | interferon regulatory factor 2 binding protein like | 0.02099734 |
| | | [Source: HGNC Symbol; Acc: HGNC: 14282] | |
| ENSG00000160446 | ZDHHC12 | zinc finger DHHC-type containing 12 | 0.02150253 |
| | | [Source: HGNC Symbol; Acc: HGNC: 19159] | |
| ENSG00000063169 | BICRA | BRD4 interacting chromatin remodeling | 0.021525887 |
| | | complex associated protein | |
| | | [Source: HGNC Symbol; Acc: HGNC: 4332] | |
| ENSG00000141933 | TPGS1 | tubulin polyglutamylase complex subunit 1 | 0.021539966 |
| | | [Source: HGNC Symbol; Acc: HGNC: 25058] | |
| ENSG00000088256 | GNA11 | G protein subunit alpha 11 | 0.021557835 |
| | | [Source: HGNC Symbol; Acc: HGNC: 4379] | |
| ENSG00000169583 | CLIC3 | chloride intracellular channel 3 | 0.021557835 |
| | | [Source: HGNC Symbol; Acc: HGNC: 2064] | |
| ENSG00000188511 | C22orf34 | chromosome 22 open reading frame 34 | 0.021557835 |
| | | [Source: HGNC Symbol; Acc: HGNC: 28010] | |
| ENSG00000165406 | 8-Mar | membrane associated ring-CH-type finger 8 | 0.021577416 |
| | | [Source: HGNC Symbol; Acc: HGNC: 23356] | |
| ENSG00000173762 | CD7 | CD7 molecule | 0.021879276 |
| | | [Source: HGNC Symbol; Acc: HGNC: 1695] | |
| ENSG00000188322 | SBK1 | SH3 domain binding kinase 1 | 0.021879276 |
| | | [Source: HGNC Symbol; Acc: HGNC: 17699] | |
| ENSG00000204310 | AGPAT1 | 1-acylglycerol-3-phosphate O-acyltransferase 1 | 0.021879276 |
| | | [Source: HGNC Symbol; Acc: HGNC: 324] | |
| ENSG00000167797 | CDK2AP2 | cyclin dependent kinase 2 associated protein 2 | 0.021895705 |
| | | [Source: HGNC Symbol; Acc: HGNC: 30833] | |
| ENSG00000142669 | SH3BGRL3 | SH3 domain binding glutamate rich protein like 3 | 0.022007819 |
| | | [Source: HGNC Symbol; Acc: HGNC: 15568] | |
| ENSG00000155034 | FBXL18 | F-box and leucine rich repeat protein 18 | 0.022133956 |
| | | [Source: HGNC Symbol; Acc: HGNC: 21874] | |
| ENSG00000187840 | EIF4EBP1 | eukaryotic translation initiation factor 4E | 0.02227554 |
| | | binding protein 1 | |
| | | [Source: HGNC Symbol; Acc: HGNC: 3288] | |
| ENSG00000185187 | SIGIRR | single Ig and TIR domain containing | 0.022615882 |
| | | [Source: HGNC Symbol; Acc: HGNC: 30575] | |
| ENSG00000158545 | ZC3H18 | zinc finger CCCH-type containing 18 | 0.022649486 |
| | | [Source: HGNC Symbol; Acc: HGNC: 25091] | |
| ENSG00000184730 | APOBR | polipoprotein B receptor | 0.022689972 |
| | | [Source: HGNC Symbol; Acc: HGNC: 24087] | |
| ENSG00000204463 | BAG6 | BCL2 associated athanogene 6 | 0.022689972 |
| | | [Source: HGNC Symbol; Acc: HGNC: 13919] | |
| ENSG00000071242 | RPS6KA2 | ribosomal protein S6 kinase A2 | 0.022776502 |
| | | [Source: HGNC Symbol; Acc: HGNC: 10431] | |
| ENSG00000146701 | MDH2 | malate dehydrogenase 2 | 0.02289018 |
| | | [Source: HGNC Symbol; Acc: HGNC: 6971] | |
| ENSG00000180155 | LYNX1 | Ly6/neurotoxin 1 | 0.02289018 |
| | | [Source: HGNC Symbol; Acc: HGNC: 29604] | |
| ENSG00000213563 | C8orf82 | chromosome 8 open reading frame 82 | 0.023112388 |
| | | [Source: HGNC Symbol; Acc: HGNC: 33826] | |
| ENSG00000105281 | SLC1A5 | solute carrier family 1 member 5 | 0.023356543 |
| | | [Source: HGNC Symbol; Acc: HGNC: 10943] | |
| ENSG00000162882 | HAAO | “3-hydroxyanthranilate 3,4-dioxygenase | 0.02337834 |
| | | [Source: HGNC Symbol; Acc: HGNC: 4796]” | |
| ENSG00000181513 | ACBD4 | acyl-CoA binding domain containing 4 | 0.02337834 |
| | | [Source: HGNC Symbol; Acc: HGNC: 23337] | |
| ENSG00000185730 | ZNF696 | zinc finger protein 696 | 0.02337834 |
| | | [Source: HGNC Symbol; Acc: HGNC: 25872] | |
| ENSG00000007520 | TSR3 | “TSR3, acp transferase ribosome maturation | 0.023637109 |
| | | factor | |
| | | [Source: HGNC Symbol; Acc: HGNC: 14175]” | |
| ENSG00000090006 | LTBP4 | latent transforming growth factor beta binding | 0.023637109 |
| | | protein 4 | |
| | | [Source: HGNC Symbol; Acc: HGNC: 6717] | |
| ENSG00000146535 | GNA12 | G protein subunit alpha 12 | 0.023652058 |
| | | [Source: HGNC Symbol; Acc: HGNC: 4380] | |
| ENSG00000141965 | FEM1A | fem-1 homolog A | 0.023707215 |
| | | [Source: HGNC Symbol; Acc: HGNC: 16934] | |
| ENSG00000160957 | RECQL4 | RecQ like helicase 4 | 0.023710452 |
| | | [Source: HGNC Symbol; Acc: HGNC: 9949] | |
| ENSG00000135916 | ITM2C | integral membrane protein 2C | 0.023733416 |
| | | [Source: HGNC Symbol; Acc: HGNC: 6175] | |
| ENSG00000177732 | SOX12 | SRY-box 12 | 0.023733416 |
| | | [Source: HGNC Symbol; Acc: HGNC: 11198] | |
| ENSG00000184508 | HDDC3 | HD domain containing 3 | 0.023802652 |
| | | [Source: HGNC Symbol; Acc: HGNC: 30522] | |
| ENSG00000175591 | P2RY2 | purinergic receptor P2Y2 | 0.023918507 |
| | | [Source: HGNC Symbol; Acc: HGNC: 8541] | |
| ENSG00000127903 | ZNF835 | zinc finger protein 835 | 0.023926586 |
| | | [Source: HGNC Symbol; Acc: HGNC: 34332] | |
| ENSG00000176022 | B3GALT6 | “beta-1,3-galactosyltransferase 6 | 0.023926586 |
| | | [Source: HGNC Symbol; Acc: HGNC: 17978]” | |
| ENSG00000255319 | ENPP7P8 | ectonucleotide pyrophosphatase/phosphodiesterase | 0.02392953 |
| | | 7 pseudogene 8 | |
| | | [Source: HGNC Symbol; Acc: HGNC: 48691] | |
| ENSG00000105479 | CCDC114 | coiled-coil domain containing 114 | 0.02398767 |
| | | [Source: HGNC Symbol; Acc: HGNC: 26560] | |
| ENSG00000130529 | TRPM4 | transient receptor potential cation channel | 0.024020687 |
| | | subfamily M member 4 | |
| | | [Source: HGNC Symbol; Acc: HGNC: 17993] | |
| ENSG00000133250 | ZNF414 | zinc finger protein 414 | 0.024020687 |
| | | [Source: HGNC Symbol; Acc: HGNC: 20630] | |
| ENSG00000215908 | CROCCP2 | “ciliary rootlet coiled-coil, rootletin pseudogene 2 | 0.024052859 |
| | | [Source: HGNC Symbol; Acc: HGNC: 28170]” | |
| ENSG00000118046 | STK11 | serine/threonine kinase 11 | 0.024056107 |
| | | [Source: HGNC Symbol; Acc: HGNC: 11389] | |
| ENSG00000034152 | MAP2K3 | mitogen-activated protein kinase kinase 3 | 0.024155164 |
| | | [Source: HGNC Symbol; Acc: HGNC: 6843] | |
| ENSG00000142453 | CARM1 | coactivator associated arginine methyltransferase 1 | 0.024155164 |
| | | [Source: HGNC Symbol; Acc: HGNC: 23393] | |
| ENSG00000256323 | NA | NA | 0.024155164 |
| ENSG00000160094 | ZNF362 | zinc finger protein 362 | 0.024171066 |
| | | [Source: HGNC Symbol; Acc: HGNC: 18079] | |
| ENSG00000104884 | ERCC2 | “ERCC excision repair 2, TFIIH core complex | 0.024345083 |
| | | helicase subunit | |
| | | [Source: HGNC Symbol; Acc: HGNC: 3434]” | |
| ENSG00000149257 | SERPINH1 | serpin family H member 1 | 0.024345083 |
| | | [Source: HGNC Symbol; Acc: HGNC: 1546] | |
| ENSG00000169635 | HIC2 | HIC ZBTB transcriptional repressor 2 | 0.024354637 |
| | | [Source: HGNC Symbol; Acc: HGNC: 18595] | |
| ENSG00000143416 | SELENBP1 | selenium binding protein 1 | 0.024421599 |
| | | [Source: HGNC Symbol; Acc: HGNC: 10719] | |
| ENSG00000148411 | NACC2 | NACC family member 2 | 0.02443315 |
| | | [Source: HGNC Symbol; Acc: HGNC: 23846] | |
| ENSG00000085872 | CHERP | calcium homeostasis endoplasmic reticulum protein | 0.024463522 |
| | | [Source: HGNC Symbol; Acc: HGNC: 16930] | |
| ENSG00000176182 | MYPOP | “Myb related transcription factor, partner of profiling | 0.024477311 |
| | | [Source: HGNC Symbol; Acc: HGNC: 20178]” | |
| ENSG00000160113 | NR2F6 | nuclear receptor subfamily 2 group F member 6 | 0.024505177 |
| | | [Source: HGNC Symbol; Acc: HGNC: 7977] | |
| ENSG00000108262 | GIT1 | GIT ArfGAP 1 | 0.024614621 |
| | | [Source: HGNC Symbol; Acc: HGNC: 4272] | |
| ENSG00000161395 | PGAP3 | post-GPI attachment to proteins 3 | 0.024705126 |
| | | [Source: HGNC Symbol; Acc: HGNC: 23719] | |
| ENSG00000142089 | IFITM3 | interferon induced transmembrane protein 3 | 0.024765171 |
| | | [Source: HGNC Symbol; Acc: HGNC: 5414] | |
| ENSG00000070444 | MNT | MAX network transcriptional repressor | 0.025148395 |
| | | [Source: HGNC Symbol; Acc: HGNC: 7188] | |
| ENSG00000112514 | CUTA | cutA divalent cation tolerance homolog | 0.025148395 |
| | | [Source: HGNC Symbol; Acc: HGNC: 21101] | |
| ENSG00000167394 | ZNF668 | zinc finger protein 668 | 0.025148395 |
| | | [Source: HGNC Symbol; Acc: HGNC: 25821] | |
| ENSG00000167965 | MLST8 | “MTOR associated protein, LST8 homolog | 0.025148395 |
| | | [Source: HGNC Symbol; Acc: HGNC: 24825]” | |
| ENSG00000244187 | TMEM141 | transmembrane protein 141 | 0.025148395 |
| | | [Source: HGNC Symbol; Acc: HGNC: 28211] | |
| ENSG00000218175 | | | 0.02527518 |
| ENSG00000110063 | DCPS | “decapping enzyme, scavenger | 0.025300524 |
| | | [Source: HGNC Symbol; Acc: HGNC: 29812]” | |
| ENSG00000128805 | ARHGAP22 | Rho GTPase activating protein 22 | 0.025318552 |
| | | [Source: HGNC Symbol; Acc: HGNC: 30320] | |
| ENSG00000148400 | NOTCH1 | notch 1 | 0.025506427 |
| | | [Source: HGNC Symbol; Acc: HGNC: 7881] | |
| ENSG00000186076 | | | 0.0257616 |
| ENSG00000167470 | MIDN | midnolin | 0.02580767 |
| | | [Source: HGNC Symbol; Acc: HGNC: 16298] | |
| ENSG00000188305 | PEAK3 | PEAK family member 3 | 0.02580767 |
| | | [Source: HGNC Symbol; Acc: HGNC: 24793] | |
| ENSG00000181396 | OGFOD3 | 2-oxoglutarate and iron dependent oxygenase | 0.026198035 |
| | | domain containing 3 | |
| | | [Source: HGNC Symbol; Acc: HGNC: 26174] | |
| ENSG00000240877 | RN7SL521P | “RNA, 7SL, cytoplasmic 521, pseudogene | 0.026492508 |
| | | [Source: HGNC Symbol; Acc: HGNC: 46537]” | |
| ENSG00000130511 | SSBP4 | single stranded DNA binding protein 4 | 0.026588317 |
| | | [Source: HGNC Symbol; Acc: HGNC: 15676] | |
| ENSG00000063177 | RPL18 | ribosomal protein L18 | 0.026698079 |
| | | [Source: HGNC Symbol; Acc: HGNC: 10310] | |
| ENSG00000172663 | TMEM134 | transmembrane protein 134 | 0.026698079 |
| | | [Source: HGNC Symbol; Acc: HGNC: 26142] | |
| ENSG00000130706 | ADRM1 | adhesion regulating molecule 1 | 0.026721536 |
| | | [Source: HGNC Symbol; Acc: HGNC: 15759] | |
| ENSG00000214063 | TSPAN4 | tetraspanin 4 | 0.026759131 |
| | | [Source: HGNC Symbol; Acc: HGNC: 11859] | |
| ENSG00000161677 | JOSD2 | Josephin domain containing 2 | 0.026798474 |
| | | [Source: HGNC Symbol; Acc: HGNC: 28853] | |
| ENSG00000189060 | H1F0 | H1 histone family member 0 | 0.027109095 |
| | | [Source: HGNC Symbol; Acc: HGNC: 4714] | |
| ENSG00000256811 | | | 0.027109095 |
| ENSG00000133317 | LGALS12 | galectin 12 | 0.027164347 |
| | | [Source: HGNC Symbol; Acc: HGNC: 15788] | |
| ENSG00000012061 | ERCC1 | “ERCC excision repair 1, endonuclease | 0.027196959 |
| | | non-catalytic subunit | |
| | | [Source: HGNC Symbol; Acc: HGNC: 3433]” | |
| ENSG00000007376 | RPUSD1 | RNA pseudouridylate synthase domain containing 1 | 0.027275974 |
| | | [Source: HGNC Symbol; Acc: HGNC: 14173] | |
| ENSG00000108175 | ZMIZ1 | zinc finger MIZ-type containing 1 | 0.027331142 |
| | | [Source: HGNC Symbol; Acc: HGNC: 16493] | |
| ENSG00000132003 | ZSWIM4 | zinc finger SWIM-type containing 4 | 0.027331142 |
| | | [Source: HGNC Symbol; Acc: HGNC: 25704] | |
| ENSG00000148296 | SURF6 | surfeit 6 | 0.027362837 |
| | | [Source: HGNC Symbol; Acc: HGNC: 11478] | |
| ENSG00000186056 | MATN1-AS1 | MATN1 antisense RNA 1 | 0.02742002 |
| | | [Source: HGNC Symbol; Acc: HGNC: 40364] | |
| ENSG00000115649 | CNPPD1 | cyclin Pas1/PHO80 domain containing 1 | 0.027574972 |
| | | [Source: HGNC Symbol; Acc: HGNC: 25220] | |
| ENSG00000065057 | NTHL1 | nth like DNA glycosylase 1 | 0.027763734 |
| | | [Source: HGNC Symbol; Acc: HGNC: 8028] | |
| ENSG00000272098 | NA | NA | 0.027825114 |
| ENSG00000011009 | LYPLA2 | lysophospholipase II | 0.028071412 |
| | | [Source: HGNC Symbol; Acc: HGNC: 6738] | |
| ENSG00000110025 | SNX15 | sorting nexin 15 | 0.028074538 |
| | | [Source: HGNC Symbol; Acc: HGNC: 14978] | |
| ENSG00000095321 | CRAT | carnitine O-acetyltransferase | 0.028133383 |
| | | [Source: HGNC Symbol; Acc: HGNC: 2342] | |
| ENSG00000108515 | ENO3 | enolase 3 | 0.028133383 |
| | | [Source: HGNC Symbol; Acc: HGNC: 3354] | |
| ENSG00000123064 | DDX54 | DEAD-box helicase 54 | 0.028358899 |
| | | [Source: HGNC Symbol; Acc: HGNC: 20084] | |
| ENSG00000169564 | PCBP1 | poly(rC) binding protein 1 | 0.028645846 |
| | | [Source: HGNC Symbol; Acc: HGNC: 8647] | |
| ENSG00000171045 | TSNARE1 | t-SNARE domain containing 1 | 0.028645846 |
| | | [Source: HGNC Symbol; Acc: HGNC: 26437] | |
| ENSG00000225978 | HAR1A | highly accelerated region 1A (non-protein coding) | 0.028645846 |
| | | [Source: HGNC Symbol; Acc: HGNC: 33117] | |
| ENSG00000128283 | CDC42EP1 | CDC42 effector protein 1 | 0.028675863 |
| | | [Source: HGNC Symbol; Acc: HGNC: 17014] | |
| ENSG00000174282 | ZBTB4 | zinc finger and BTB domain containing 4 | 0.028871519 |
| | | [Source: HGNC Symbol; Acc: HGNC: 23847] | |
| ENSG00000167685 | ZNF444 | zinc finger protein 444 | 0.028919683 |
| | | [Source: HGNC Symbol; Acc: HGNC: 16052] | |
| ENSG00000110104 | CCDC86 | coiled-coil domain containing 86 | 0.028929404 |
| | | [Source: HGNC Symbol; Acc: HGNC: 28359] | |
| ENSG00000171703 | TCEA2 | transcription elongation factor A2 | 0.029117009 |
| | | [Source: HGNC Symbol; Acc: HGNC: 11614] | |
| ENSG00000177600 | RPLP2 | ribosomal protein lateral stalk subunit P2 | 0.029298722 |
| | | [Source: HGNC Symbol; Acc: HGNC: 10377] | |
| ENSG00000182095 | TNRC18 | trinucleotide repeat containing 18 | 0.029299181 |
| | | [Source: HGNC Symbol; Acc: HGNC: 11962] | |
| ENSG00000167106 | FAM102A | family with sequence similarity 102 member A | 0.029415219 |
| | | [Source: HGNC Symbol; Acc: HGNC: 31419] | |
| ENSG00000126458 | RRAS | RAS related | 0.02952737 |
| | | [Source: HGNC Symbol; Acc: HGNC: 10447] | |
| ENSG00000105063 | PPP6R1 | protein phosphatase 6 regulatory subunit 1 | 0.02959944 |
| | | [Source: HGNC Symbol; Acc: HGNC: 29195] | |
| ENSG00000125730 | C3 | complement C3 | 0.029997906 |
| | | [Source: HGNC Symbol; Acc: HGNC: 1318] | |
| ENSG00000237973 | MTCO1P12 | mitochondrially encoded cytochrome c oxidase | 0.030144659 |
| | | I pseudogene 12 | |
| | | [Source: HGNC Symbol; Acc: HGNC: 52014] | |
| ENSG00000267412 | | | 0.030144659 |
| ENSG00000185813 | PCYT2 | “phosphate cytidylyltransferase 2, ethanolamine | 0.030317293 |
| | | [Source: HGNC Symbol; Acc: HGNC: 8756]” | |
| ENSG00000163462 | TRIM46 | tripartite motif containing 46 | 0.030614393 |
| | | [Source: HGNC Symbol; Acc: HGNC: 19019] | |
| ENSG00000157933 | SKI | SKI proto-oncogene | 0.030701411 |
| | | [Source: HGNC Symbol; Acc: HGNC: 10896] | |
| ENSG00000161091 | MFSD12 | major facilitator superfamily domain containing 12 | 0.030704292 |
| | | [Source: HGNC Symbol; Acc: HGNC: 28299] | |
| ENSG00000185163 | DDX51 | DEAD-box helicase 51 | 0.030735268 |
| | | [Source: HGNC Symbol; Acc: HGNC: 20082] | |
| ENSG00000171813 | PWWP2B | PWWP domain containing 2B | 0.030810478 |
| | | [Source: HGNC Symbol; Acc: HGNC: 25150] | |
| ENSG00000137267 | TUBB2A | tubulin beta 2A class IIa | 0.030847037 |
| | | [Source: HGNC Symbol; Acc: HGNC: 12412] | |
| ENSG00000188747 | NOXA1 | NADPH oxidase activator 1 | 0.030853611 |
| | | [Source: HGNC Symbol; Acc: HGNC: 10668] | |
| ENSG00000108557 | RAI1 | retinoic acid induced 1 | 0.030977697 |
| | | [Source: HGNC Symbol; Acc: HGNC: 9834] | |
| ENSG00000137166 | FOXP4 | forkhead box P4 | 0.030977697 |
| | | [Source: HGNC Symbol; Acc: HGNC: 20842] | |
| ENSG00000204420 | MPIG6B | megakaryocyte and platelet inhibitory receptor G6b | 0.031369168 |
| | | [Source: HGNC Symbol; Acc: HGNC: 13937] | |
| ENSG00000133265 | HSPBP1 | HSPA (Hsp70) binding protein 1 | 0.031442624 |
| | | [Source: HGNC Symbol; Acc: HGNC: 24989] | |
| ENSG00000008710 | PKD1 | “polycystin 1, transient receptor potential | 0.031566582 |
| | | channel interacting | |
| | | [Source: HGNC Symbol; Acc: HGNC: 9008]” | |
| ENSG00000099624 | ATP5F1D | ATP synthase F1 subunit delta | 0.031657927 |
| | | [Source: HGNC Symbol; Acc: HGNC: 837] | |
| ENSG00000108819 | PPP1R9B | protein phosphatase 1 regulatory subunit 9B | 0.031695534 |
| | | [Source: HGNC Symbol; Acc: HGNC: 9298] | |
| ENSG00000158292 | GPR153 | G protein-coupled receptor 153 | 0.031750316 |
| | | [Source: HGNC Symbol; Acc: HGNC: 23618] | |
| ENSG00000130382 | MLLT1 | “MLLT1, super elongation complex subunit | 0.031828022 |
| | | [Source: HGNC Symbol; Acc: HGNC: 7134]” | |
| ENSG00000269352 | PTOV1-AS2 | PTOV1 antisense RNA 2 | 0.031850457 |
| | | [Source: HGNC Symbol; Acc: HGNC: 51284] | |
| ENSG00000162585 | FAAP20 | Fanconi anemia core complex associated protein 20 | 0.0320507 |
| | | [Source: HGNC Symbol; Acc: HGNC: 26428] | |
| ENSG00000157240 | FZD1 | frizzled class receptor 1 | 0.032151732 |
| | | [Source: HGNC Symbol; Acc: HGNC: 4038] | |
| ENSG00000135736 | CCDC102A | coiled-coil domain containing 102A | 0.032302006 |
| | | [Source: HGNC Symbol; Acc: HGNC: 28097] | |
| ENSG00000020181 | ADGRA2 | adhesion G protein-coupled receptor A2 | 0.032424937 |
| | | [Source: HGNC Symbol; Acc: HGNC: 17849] | |
| ENSG00000198546 | ZNF511 | zinc finger protein 511 | 0.032576231 |
| | | [Source: HGNC Symbol; Acc: HGNC: 28445] | |
| ENSG00000123144 | TRIR | telomerase RNA component interacting RNase | 0.032671184 |
| | | [Source: HGNC Symbol; Acc: HGNC: 28424] | |
| ENSG00000156860 | FBRS | fibrosin | 0.032671184 |
| | | [Source: HGNC Symbol; Acc: HGNC: 20442] | |
| ENSG00000162910 | MRPL55 | mitochondrial ribosomal protein L55 | 0.032697662 |
| | | [Source: HGNC Symbol; Acc: HGNC: 16686] | |
| ENSG00000130731 | METTL26 | methyltransferase like 26 | 0.032833764 |
| | | [Source: HGNC Symbol; Acc: HGNC: 14141] | |
| ENSG00000101986 | ABCD1 | ATP binding cassette subfamily D member 1 | 0.032886018 |
| | | [Source: HGNC Symbol; Acc: HGNC: 61] | |
| ENSG00000020633 | RUNX3 | runt related transcription factor 3 | 0.033016587 |
| | | [Source: HGNC Symbol; Acc: HGNC: 10473] | |
| ENSG00000184640 | 9-Sep | septin 9 | 0.033101928 |
| | | [Source: HGNC Symbol; Acc: HGNC: 7323] | |
| ENSG00000260521 | NA | NA | 0.033101928 |
| ENSG00000125787 | GNRH2 | gonadotropin releasing hormone 2 | 0.033349502 |
| | | [Source: HGNC Symbol; Acc: HGNC: 4420] | |
| ENSG00000229391 | HLA-DRB6 | “major histocompatibility complex, class II, | 0.033349502 |
| | | DR beta 6 (pseudogene) | |
| | | [Source: HGNC Symbol; Acc: HGNC: 4954]” | |
| ENSG00000160223 | ICOSLG | inducible T cell costimulator ligand | 0.033394348 |
| | | [Source: HGNC Symbol; Acc: HGNC: 17087] | |
| ENSG00000105204 | DYRK1B | dual specificity tyrosine phosphorylation | 0.033465567 |
| | | regulated kinase 1B | |
| | | [Source: HGNC Symbol; Acc: HGNC: 3092] | |
| ENSG00000142173 | COL6A2 | collagen type VI alpha 2 chain | 0.033483825 |
| | | [Source: HGNC Symbol; Acc: HGNC: 2212] | |
| ENSG00000169710 | FASN | fatty acid synthase | 0.033483825 |
| | | [Source: HGNC Symbol; Acc: HGNC: 3594] | |
| ENSG00000176533 | GNG7 | G protein subunit gamma 7 | 0.033483825 |
| | | [Source: HGNC Symbol; Acc: HGNC: 4410] | |
| ENSG00000179253 | | | 0.033483825 |
| ENSG00000169972 | PUSL1 | pseudouridylate synthase-like 1 | 0.033509544 |
| | | [Source: HGNC Symbol; Acc: HGNC: 26914] | |
| ENSG00000160360 | GPSM1 | G protein signaling modulator 1 | 0.033771567 |
| | | [Source: HGNC Symbol; Acc: HGNC: 17858] | |
| ENSG00000171159 | C9orf16 | chromosome 9 open reading frame 16 | 0.033853634 |
| | | [Source: HGNC Symbol; Acc: HGNC: 17823] | |
| ENSG00000215375 | MYL5 | myosin light chain 5 | 0.033853634 |
| | | [Source: HGNC Symbol; Acc: HGNC: 7586] | |
| ENSG00000105402 | NAPA | NSF attachment protein alpha | 0.034044441 |
| | | [Source: HGNC Symbol; Acc: HGNC: 7641] | |
| ENSG00000038532 | CLEC16A | C-type lectin domain containing 16A | 0.034108157 |
| | | [Source: HGNC Symbol; Acc: HGNC: 29013] | |
| ENSG00000165175 | MID1IP1 | MID1 interacting protein 1 | 0.03418982 |
| | | [Source: HGNC Symbol; Acc: HGNC: 20715] | |
| ENSG00000166947 | EPB42 | erythrocyte membrane protein band 4.2 | 0.03418982 |
| | | [Source: HGNC Symbol; Acc: HGNC: 3381] | |
| ENSG00000168286 | THAP11 | THAP domain containing 11 | 0.03418982 |
| | | [Source: HGNC Symbol; Acc: HGNC: 23194] | |
| ENSG00000168476 | REEP4 | receptor accessory protein 4 | 0.034591261 |
| | | [Source: HGNC Symbol; Acc: HGNC: 26176] | |
| ENSG00000107521 | HPS1 | “HPS1, biogenesis of lysosomal organelles | 0.034688533 |
| | | complex 3 subunit 1 | |
| | | [Source: HGNC Symbol; Acc: HGNC: 5163]” | |
| ENSG00000267436 | | | 0.034821851 |
| ENSG00000099991 | CABIN1 | calcineurin binding protein 1 | 0.034858474 |
| | | [Source: HGNC Symbol; Acc: HGNC: 24187] | |
| ENSG00000169718 | DUS1L | dihydrouridine synthase 1 like | 0.034980702 |
| | | [Source: HGNC Symbol; Acc: HGNC: 30086] | |
| ENSG00000105325 | FZR1 | fizzy and cell division cycle 20 related 1 | 0.035034499 |
| | | [Source: HGNC Symbol; Acc: HGNC: 24824] | |
| ENSG00000167291 | TBC1D16 | TBC1 domain family member 16 | 0.035082388 |
| | | [Source: HGNC Symbol; Acc: HGNC: 28356] | |
| ENSG00000213399 | | | 0.035085538 |
| ENSG00000175040 | CHST2 | carbohydrate sulfotransferase 2 | 0.035106231 |
| | | [Source: HGNC Symbol; Acc: HGNC: 1970] | |
| ENSG00000228544 | CCDC183-AS1 | CCDC183 antisense RNA 1 | 0.035154264 |
| | | [Source: HGNC Symbol; Acc: HGNC: 44105] | |
| ENSG00000167658 | EEF2 | eukaryotic translation elongation factor 2 | 0.03521308 |
| | | [Source: HGNC Symbol; Acc: HGNC: 3214] | |
| ENSG00000090238 | YPEL3 | yippee like 3 | 0.035223199 |
| | | [Source: HGNC Symbol; Acc: HGNC: 18327] | |
| ENSG00000172508 | CARNS1 | carnosine synthase 1 | 0.03555645 |
| | | [Source: HGNC Symbol; Acc: HGNC: 29268] | |
| ENSG00000173272 | MZT2A | mitotic spindle organizing protein 2A | 0.035664739 |
| | | [Source: HGNC Symbol; Acc: HGNC: 33187] | |
| ENSG00000141522 | ARHGDIA | Rho GDP dissociation inhibitor alpha | 0.03575577 |
| | | [Source: HGNC Symbol; Acc: HGNC: 678] | |
| ENSG00000149541 | B3GAT3 | “beta-1,3-glucuronyltransferase 3 | 0.03575577 |
| | | [Source: HGNC Symbol; Acc: HGNC: 923]” | |
| ENSG00000171206 | TRIM8 | tripartite motif containing 8 | 0.035790036 |
| | | [Source: HGNC Symbol; Acc: HGNC: 15579] | |
| ENSG00000027869 | SH2D2A | SH2 domain containing 2A | 0.035871776 |
| | | [Source: HGNC Symbol; Acc: HGNC: 10821] | |
| ENSG00000149823 | VPS51 | “VPS51, GARP complex subunit | 0.035906645 |
| | | [Source: HGNC Symbol; Acc: HGNC: 1172]” | |
| ENSG00000196355 | NA | NA | 0.035938935 |
| ENSG00000165804 | ZNF219 | zinc finger protein 219 | 0.035996581 |
| | | [Source: HGNC Symbol; Acc: HGNC: 13011] | |
| ENSG00000177542 | SLC25A22 | solute carrier family 25 member 22 | 0.035996581 |
| | | [Source: HGNC Symbol; Acc: HGNC: 19954] | |
| ENSG00000130202 | NECTIN2 | nectin cell adhesion molecule 2 | 0.036025366 |
| | | [Source: HGNC Symbol; Acc: HGNC: 9707] | |
| ENSG00000006638 | TBXA2R | thromboxane A2 receptor | 0.036065767 |
| | | [Source: HGNC Symbol; Acc: HGNC: 11608] | |
| ENSG00000025770 | NCAPH2 | non-SMC condensin II complex subunit H2 | 0.036065767 |
| | | [Source: HGNC Symbol; Acc: HGNC: 25071] | |
| ENSG00000100316 | RPL3 | ribosomal protein L3 | 0.036065767 |
| | | [Source: HGNC Symbol; Acc: HGNC: 10332] | |
| ENSG00000110665 | C11orf21 | chromosome 11 open reading frame 21 | 0.036065767 |
| | | [Source: HGNC Symbol; Acc: HGNC: 13231] | |
| ENSG00000160445 | ZER1 | zyg-11 related cell cycle regulator | 0.036065767 |
| | | [Source: HGNC Symbol; Acc: HGNC: 30960] | |
| ENSG00000173786 | CNP | “2′,3′-cyclic nucleotide 3′ phosphodiesterase | 0.036065767 |
| | | [Source: HGNC Symbol; Acc: HGNC: 2158]” | |
| ENSG00000229368 | | | 0.036065767 |
| ENSG00000160789 | LMNA | lamin A/C | 0.036188381 |
| | | [Source: HGNC Symbol; Acc: HGNC: 6636] | |
| ENSG00000166189 | HPS6 | “HPS6, biogenesis of lysosomal organelles | 0.036188381 |
| | | complex 2 subunit 3 | |
| | | [Source: HGNC Symbol; Acc: HGNC: 18817]” | |
| ENSG00000261226 | | | 0.036342968 |
| ENSG00000185049 | NELFA | negative elongation factor complex member A | 0.036383164 |
| | | [Source: HGNC Symbol; Acc: HGNC: 12768] | |
| ENSG00000005882 | PDK2 | pyruvate dehydrogenase kinase 2 | 0.036423123 |
| | | [Source: HGNC Symbol; Acc: HGNC: 8810] | |
| ENSG00000163050 | COQ8A | coenzyme Q8A | 0.036459828 |
| | | [Source: HGNC Symbol; Acc: HGNC: 16812] | |
| ENSG00000060138 | YBX3 | Y-box binding protein 3 | 0.03651176 |
| | | [Source: HGNC Symbol; Acc: HGNC: 2428] | |
| ENSG00000122971 | ACADS | acyl-CoA dehydrogenase short chain | 0.036511776 |
| | | [Source: HGNC Symbol; Acc: HGNC: 90] | |
| ENSG00000205927 | OLIG2 | oligodendrocyte transcription factor 2 | 0.036582868 |
| | | [Source: HGNC Symbol; Acc: HGNC: 9398] | |
| ENSG00000092096 | SLC22A17 | solute carrier family 22 member 17 | 0.036619295 |
| | | [Source: HGNC Symbol; Acc: HGNC: 23095] | |
| ENSG00000090554 | FLT3LG | fms related tyrosine kinase 3 ligand | 0.036780281 |
| | | [Source: HGNC Symbol; Acc: HGNC: 3766] | |
| ENSG00000078902 | TOLLIP | toll interacting protein | 0.036796695 |
| | | [Source: HGNC Symbol; Acc: HGNC: 16476] | |
| ENSG00000136802 | LRRC8A | leucine rich repeat containing 8 VRAC subunit A | 0.036860551 |
| | | [Source: HGNC Symbol; Acc: HGNC: 19027] | |
| ENSG00000236976 | | | 0.036972133 |
| ENSG00000100908 | EMC9 | ER membrane protein complex subunit 9 | 0.037054085 |
| | | [Source: HGNC Symbol; Acc: HGNC: 20273] | |
| ENSG00000105364 | MRPL4 | mitochondrial ribosomal protein L4 | 0.03712252 |
| | | [Source: HGNC Symbol; Acc: HGNC: 14276] | |
| ENSG00000157184 | CPT2 | carnitine palmitoyltransferase 2 | 0.03712252 |
| | | [Source: HGNC Symbol; Acc: HGNC: 2330] | |
| ENSG00000168056 | LTBP3 | latent transforming growth factor beta binding | 0.037135417 |
| | | protein 3 | |
| | | [Source: HGNC Symbol; Acc: HGNC: 6716] | |
| ENSG00000196313 | POM121 | POM121 transmembrane nucleoporin | 0.037140956 |
| | | [Source: HGNC Symbol; Acc: HGNC: 19702] | |
| ENSG00000170604 | IRF2BP1 | nterferon regulatory factor 2 binding protein 1 | 0.037222007 |
| | | [Source: HGNC Symbol; Acc: HGNC: 21728] | |
| ENSG00000110697 | PITPNM1 | phosphatidylinositol transfer protein membrane | 0.037374978 |
| | | associated 1 | |
| | | [Source: HGNC Symbol; Acc: HGNC: 9003] | |
| ENSG00000100348 | TXN2 | thioredoxin 2 | 0.037546969 |
| | | [Source: HGNC Symbol; Acc: HGNC: 17772] | |
| ENSG00000102007 | PLP2 | proteolipid protein 2 | 0.037630556 |
| | | [Source: HGNC Symbol; Acc: HGNC: 9087] | |
| ENSG00000132005 | RFX1 | regulatory factor X1 | 0.037671165 |
| | | [Source: HGNC Symbol; Acc: HGNC: 9982] | |
| ENSG00000141499 | WRAP53 | WD repeat containing antisense to TP53 | 0.037678229 |
| | | [Source: HGNC Symbol; Acc: HGNC: 25522] | |
| ENSG00000189114 | BLOC1S3 | biogenesis of lysosomal organelles complex 1 | 0.037963219 |
| | | subunit 3 | |
| | | [Source: HGNC Symbol; Acc: HGNC: 20914] | |
| ENSG00000123154 | WDR83 | WD repeat domain 83 | 0.038059131 |
| | | [Source: HGNC Symbol; Acc: HGNC: 32672] | |
| ENSG00000127663 | KDM4B | lysine demethylase 4B | 0.038059131 |
| | | [Source: HGNC Symbol; Acc: HGNC: 29136] | |
| ENSG00000175274 | TP53I11 | tumor protein p53 inducible protein 11 | 0.038059131 |
| | | [Source: HGNC Symbol; Acc: HGNC: 16842] | |
| ENSG00000249115 | HAUS5 | HAUS augmin like complex subunit 5 | 0.038059131 |
| | | [Source: HGNC Symbol; Acc: HGNC: 29130] | |
| ENSG00000130764 | LRRC47 | leucine rich repeat containing 47 | 0.038286929 |
| | | [Source: HGNC Symbol; Acc: HGNC: 29207] | |
| ENSG00000176946 | THAP4 | THAP domain containing 4 | 0.038286929 |
| | | [Source: HGNC Symbol; Acc: HGNC: 23187] | |
| ENSG00000137497 | NUMA1 | nuclear mitotic apparatus protein 1 | 0.038678067 |
| | | [Source: HGNC Symbol; Acc: HGNC: 8059] | |
| ENSG00000143761 | ARF1 | ADP ribosylation factor 1 | 0.038678067 |
| | | [Source: HGNC Symbol; Acc: HGNC: 652] | |
| ENSG00000198931 | APRT | adenine phosphoribosyltransferase | 0.038678067 |
| | | [Source: HGNC Symbol; Acc: HGNC: 626] | |
| ENSG00000186174 | BCL9L | B cell CLL/lymphoma 9 like | 0.039375488 |
| | | [Source: HGNC Symbol; Acc: HGNC: 23688] | |
| ENSG00000104894 | CD37 | CD37 molecule | 0.039523734 |
| | | [Source: HGNC Symbol; Acc: HGNC: 1666] | |
| ENSG00000235314 | LINC00957 | long intergenic non-protein coding RNA 957 | 0.039534059 |
| | | [Source: HGNC Symbol; Acc: HGNC: 22332] | |
| ENSG00000076864 | RAP1GAP | RAP1 GTPase activating protein | 0.039534171 |
| | | [Source: HGNC Symbol; Acc: HGNC: 9858] | |
| ENSG00000179348 | GATA2 | GATA binding protein 2 | 0.039644975 |
| | | [Source: HGNC Symbol; Acc: HGNC: 4171] | |
| ENSG00000223496 | EXOSC6 | exosome component 6 | 0.039646513 |
| | | [Source: HGNC Symbol; Acc: HGNC: 19055] | |
| ENSG00000174004 | NRROS | negative regulator of reactive oxygen species | 0.039697731 |
| | | [Source: HGNC Symbol; Acc: HGNC: 24613] | |
| ENSG00000185736 | ADARB2 | “adenosine deaminase, RNA specific B2 (inactive) | 0.039813584 |
| | | [Source: HGNC Symbol; Acc: HGNC: 227]” | |
| ENSG00000177595 | PIDD1 | p53-induced death domain protein 1 | 0.039841437 |
| | | [Source: HGNC Symbol; Acc: HGNC: 16491] | |
| ENSG00000114767 | RRP9 | “ribosomal RNA processing 9, U3 small | 0.039852243 |
| | | nucleolar RNA binding protein | |
| | | [Source: HGNC Symbol; Acc: HGNC: 16829]” | |
| ENSG00000198336 | MYL4 | myosin light chain 4 | 0.040061329 |
| | | [Source: HGNC Symbol; Acc: HGNC: 7585] | |
| ENSG00000267427 | NA | NA | 0.040061329 |
| ENSG00000123159 | GIPC1 | GIPC PDZ domain containing family member 1 | 0.040207239 |
| | | [Source: HGNC Symbol; Acc: HGNC: 1226] | |
| ENSG00000139405 | RITA1 | RBPJ interacting and tubulin associated 1 | 0.040571361 |
| | | [Source: HGNC Symbol; Acc: HGNC: 25925] | |
| ENSG00000149929 | HIRIP3 | HIRA interacting protein 3 | 0.040590193 |
| | | [Source: HGNC Symbol; Acc: HGNC: 4917] | |
| ENSG00000198517 | MAFK | MAF bZIP transcription factor K | 0.040590193 |
| | | [Source: HGNC Symbol; Acc: HGNC: 6782] | |
| ENSG00000164897 | TMUB1 | transmembrane and ubiquitin like domain | 0.040938395 |
| | | containing 1 | |
| | | [Source: HGNC Symbol; Acc: HGNC: 21709] | |
| ENSG00000070047 | PHRF1 | PHD and ring finger domains 1 | 0.041015111 |
| | | [Source: HGNC Symbol; Acc: HGNC: 24351] | |
| ENSG00000100403 | ZC3H7B | zinc finger CCCH-type containing 7B | 0.041151359 |
| | | [Source: HGNC Symbol; Acc: HGNC: 30869] | |
| ENSG00000205147 | NA | NA | 0.041207854 |
| ENSG00000184470 | TXNRD2 | thioredoxin reductase 2 | 0.04134465 |
| | | [Source: HGNC Symbol; Acc: HGNC: 18155] | |
| ENSG00000103145 | HCFC1R1 | host cell factor C1 regulator 1 | 0.041366672 |
| | | [Source: HGNC Symbol; Acc: HGNC: 21198] | |
| ENSG00000087086 | FTL | ferritin light chain | 0.041631474 |
| | | [Source: HGNC Symbol; Acc: HGNC: 3999] | |
| ENSG00000102870 | ZNF629 | zinc finger protein 629 | 0.041631474 |
| | | [Source: HGNC Symbol; Acc: HGNC: 29008] | |
| ENSG00000181444 | ZNF467 | zinc finger protein 467 | 0.041868067 |
| | | [Source: HGNC Symbol; Acc: HGNC: 23154] | |
| ENSG00000142444 | TIMM29 | translocase of inner mitochondrial membrane 29 | 0.041933219 |
| | | [Source: HGNC Symbol; Acc: HGNC: 25152] | |
| ENSG00000204252 | HLA-DOA | “major histocompatibility complex, class II, DO alpha | 0.041944166 |
| | | [Source: HGNC Symbol; Acc: HGNC: 4936]” | |
| ENSG00000224051 | CPTP | ceramide-1-phosphate transfer protein | 0.041944166 |
| | | [Source: HGNC Symbol; Acc: HGNC: 28116] | |
| ENSG00000103253 | HAGHL | hydroxyacylglutathione hydrolase like | 0.042027801 |
| | | [Source: HGNC Symbol; Acc: HGNC: 14177] | |
| ENSG00000011590 | ZBTB32 | zinc finger and BTB domain containing 32 | 0.042101327 |
| | | [Source: HGNC Symbol; Acc: HGNC: 16763] | |
| ENSG00000182566 | CLEC4G | C-type lectin domain family 4 member G | 0.042114834 |
| | | [Source: HGNC Symbol; Acc: HGNC: 24591] | |
| ENSG00000244165 | P2RY11 | purinergic receptor P2Y11 | 0.042114834 |
| | | [Source: HGNC Symbol; Acc: HGNC: 8540] | |
| ENSG00000267275 | | | 0.042114834 |
| ENSG00000173327 | MAP3K11 | mitogen-activated protein kinase kinase kinase 11 | 0.042395519 |
| | | [Source: HGNC Symbol; Acc: HGNC: 6850] | |
| ENSG00000149418 | ST14 | suppression of tumorigenicity 14 | 0.04243773 |
| | | [Source: HGNC Symbol; Acc: HGNC: 11344] | |
| ENSG00000112658 | SRF | serum response factor | 0.042440717 |
| | | [Source: HGNC Symbol; Acc: HGNC: 11291] | |
| ENSG00000106003 | LFNG | LFNG O-fucosylpeptide | 0.042450091 |
| | | 3-beta-N-acetylglucosaminyltransferase | |
| | | [Source: HGNC Symbol; Acc: HGNC: 6560] | |
| ENSG00000164896 | FASTK | Fas activated serine/threonine kinase | 0.042450091 |
| | | [Source: HGNC Symbol; Acc: HGNC: 24676] | |
| ENSG00000196544 | BORCS6 | BLOC-1 related complex subunit 6 | 0.042514351 |
| | | [Source: HGNC Symbol; Acc: HGNC: 25939] | |
| ENSG00000134107 | BHLHE40 | basic helix-loop-helix family member e40 | 0.042517218 |
| | | [Source: HGNC Symbol; Acc: HGNC: 1046] | |
| ENSG00000151176 | PLBD2 | phospholipase B domain containing 2 | 0.042582368 |
| | | [Source: HGNC Symbol; Acc: HGNC: 27283] | |
| ENSG00000183397 | C19orf71 | chromosome 19 open reading frame 71 | 0.042902315 |
| | | [Source: HGNC Symbol; Acc: HGNC: 34496] | |
| ENSG00000105738 | SIPA1L3 | signal induced proliferation associated 1 like 3 | 0.043052047 |
| | | [Source: HGNC Symbol; Acc: HGNC: 23801] | |
| ENSG00000157353 | FUK | fucokinase | 0.043381308 |
| | | [Source: HGNC Symbol; Acc: HGNC: 29500] | |
| ENSG00000126062 | TMEM115 | transmembrane protein 115 | 0.043382452 |
| | | [Source: HGNC Symbol; Acc: HGNC: 30055] | |
| ENSG00000179632 | MAF1 | “MAF1 homolog, negative regulator of RNA | 0.043386409 |
| | | polymerase III | |
| | | [Source: HGNC Symbol; Acc: HGNC: 24966]” | |
| ENSG00000254910 | | | 0.043544967 |
| ENSG00000073111 | MCM2 | minichromosome maintenance complex component 2 | 0.043556269 |
| | | [Source: HGNC Symbol; Acc: HGNC: 6944] | |
| ENSG00000105698 | USF2 | “upstream transcription factor 2, c-fos interacting | 0.043737282 |
| | | [Source: HGNC Symbol; Acc: HGNC: 12594]” | |
| ENSG00000261043 | | | 0.043870446 |
| ENSG00000078808 | SDF4 | stromal cell derived factor 4 | 0.043914575 |
| | | [Source: HGNC Symbol; Acc: HGNC: 24188] | |
| ENSG00000182154 | MRPL41 | mitochondrial ribosomal protein L41 | 0.044022575 |
| | | [Source: HGNC Symbol; Acc: HGNC: 14492] | |
| ENSG00000237476 | LINC01637 | long intergenic non-protein coding RNA 1637 | 0.044022575 |
| | | [Source: HGNC Symbol; Acc: HGNC: 52424] | |
| ENSG00000264577 | | | 0.044022575 |
| ENSG00000160877 | NACC1 | nucleus accumbens associated 1 | 0.044046233 |
| | | [Source: HGNC Symbol; Acc: HGNC: 20967] | |
| ENSG00000162032 | SPSB3 | splA/ryanodine receptor domain and SOCS box | 0.044046233 |
| | | containing 3 | |
| | | [Source: HGNC Symbol; Acc: HGNC: 30629] | |
| ENSG00000123933 | MXD4 | MAX dimerization protein 4 | 0.044146857 |
| | | [Source: HGNC Symbol; Acc: HGNC: 13906] | |
| ENSG00000149260 | CAPN5 | calpain 5 | 0.044146857 |
| | | [Source: HGNC Symbol; Acc: HGNC: 1482] | |
| ENSG00000197324 | LRP10 | LDL receptor related protein 10 | 0.044146857 |
| | | [Source: HGNC Symbol; Acc: HGNC: 14553] | |
| ENSG00000128011 | LRFN1 | leucine rich repeat and fibronectin type III | 0.044424476 |
| | | domain containing 1 | |
| | | [Source: HGNC Symbol; Acc: HGNC: 29290] | |
| ENSG00000103126 | AXIN1 | axin 1 | 0.044490553 |
| | | [Source: HGNC Symbol; Acc: HGNC: 903] | |
| ENSG00000183134 | PTGDR2 | prostaglandin D2 receptor 2 | 0.044490553 |
| | | [Source: HGNC Symbol; Acc: HGNC: 4502] | |
| ENSG00000185905 | C16orf54 | chromosome 16 open reading frame 54 | 0.044574634 |
| | | [Source: HGNC Symbol; Acc: HGNC: 26649] | |
| ENSG00000185905 | C16orf54 | chromosome 16 open reading frame 54 | 0.044574634 |
| | | [Source: HGNC Symbol; Acc: HGNC: 26649] | |
| ENSG00000176974 | SHMT1 | serine hydroxymethyltransferase 1 | 0.044717969 |
| | | [Source: HGNC Symbol; Acc: HGNC: 10850] | |
| ENSG00000105327 | BBC3 | BCL2 binding component 3 | 0.044899327 |
| | | [Source: HGNC Symbol; Acc: HGNC: 17868] | |
| ENSG00000212123 | PRR22 | proline rich 22 | 0.044899327 |
| | | [Source: HGNC Symbol; Acc: HGNC: 28354] | |
| ENSG00000171798 | KNDC1 | kinase non-catalytic C-lobe domain containing 1 | 0.045139854 |
| | | [Source: HGNC Symbol; Acc: HGNC: 29374] | |
| ENSG00000073150 | PANX2 | pannexin 2 | 0.045237232 |
| | | [Source: HGNC Symbol; Acc: HGNC: 8600] | |
| ENSG00000126453 | BCL2L12 | BCL2 like 12 | 0.04527356 |
| | | [Source: HGNC Symbol; Acc: HGNC: 13787] | |
| ENSG00000159692 | CTBP1 | C-terminal binding protein 1 | 0.04527356 |
| | | [Source: HGNC Symbol; Acc: HGNC: 2494] | |
| ENSG00000105248 | CCDC94 | coiled-coil domain containing 94 | 0.045450088 |
| | | [Source: HGNC Symbol; Acc: HGNC: 25518] | |
| ENSG00000101220 | C20orf27 | chromosome 20 open reading frame 27 | 0.045474144 |
| | | [Source: HGNC Symbol; Acc: HGNC: 15873] | |
| ENSG00000188735 | TMEM120B | transmembrane protein 120B | 0.045474144 |
| | | [Source: HGNC Symbol; Acc: HGNC: 32008] | |
| ENSG00000149476 | TKFC | triokinase and FMN cyclase | 0.045497488 |
| | | [Source: HGNC Symbol; Acc: HGNC: 24552] | |
| ENSG00000100105 | PATZ1 | POZ/BTB and AT hook containing zinc finger 1 | 0.045500961 |
| | | [Source: HGNC Symbol; Acc: HGNC: 13071] | |
| ENSG00000136295 | TTYH3 | tweety family member 3 | 0.045528908 |
| | | [Source: HGNC Symbol; Acc: HGNC: 22222] | |
| ENSG00000104823 | ECH1 | enoyl-CoA hydratase 1 | 0.045555559 |
| | | [Source: HGNC Symbol; Acc: HGNC: 3149] | |
| ENSG00000167930 | FAM234A | family with sequence similarity 234 member A | 0.045584551 |
| | | [Source: HGNC Symbol; Acc: HGNC: 14163] | |
| ENSG00000125505 | MBOAT7 | membrane bound O-acyltransferase domain | 0.045601783 |
| | | containing 7 | |
| | | [Source: HGNC Symbol; Acc: HGNC: 15505] | |
| ENSG00000136720 | HS6ST1 | heparan sulfate 6-O-sulfotransferase 1 | 0.045601783 |
| | | [Source: HGNC Symbol; Acc: HGNC: 5201] | |
| ENSG00000156853 | ZNF689 | zinc finger protein 689 | 0.045601783 |
| | | [Source: HGNC Symbol; Acc: HGNC: 25173] | |
| ENSG00000176108 | CHMP6 | charged multivesicular body protein 6 | 0.045601783 |
| | | [Source: HGNC Symbol; Acc: HGNC: 25675] | |
| ENSG00000205336 | ADGRG1 | adhesion G protein-coupled receptor G1 | 0.045601783 |
| | | [Source: HGNC Symbol; Acc: HGNC: 4512] | |
| ENSG00000186350 | RXRA | retinoid X receptor alpha | 0.045691236 |
| | | [Source: HGNC Symbol; Acc: HGNC: 10477] | |
| ENSG00000164849 | GPR146 | G protein-coupled receptor 146 | 0.045781 |
| | | [Source: HGNC Symbol; Acc: HGNC: 21718] | |
| ENSG00000124313 | IQSEC2 | IQ motif and Sec7 domain 2 | 0.045921742 |
| | | [Source: HGNC Symbol; Acc: HGNC: 29059] | |
| ENSG00000148341 | SH3GLB2 | “SH3 domain containing GRB2 like, endophilin B2 | 0.045931004 |
| | | [Source: HGNC Symbol; Acc: HGNC: 10834]” | |
| ENSG00000213654 | GPSM3 | G protein signaling modulator 3 | 0.045963746 |
| | | [Source: HGNC Symbol; Acc: HGNC: 13945] | |
| ENSG00000171219 | CDC42BPG | CDC42 binding protein kinase gamma | 0.046017697 |
| | | [Source: HGNC Symbol; Acc: HGNC: 29829] | |
| ENSG00000125741 | OPA3 | “OPA3, outer mitochondrial membrane lipid | 0.046210538 |
| | | metabolism regulator | |
| | | [Source: HGNC Symbol; Acc: HGNC: 8142]” | |
| ENSG00000089693 | MLF2 | myeloid leukemia factor 2 | 0.046269835 |
| | | [Source: HGNC Symbol; Acc: HGNC: 7126] | |
| ENSG00000267283 | | | 0.046536749 |
| ENSG00000238164 | TNFRSF14-AS1 | TNFRSF14 antisense RNA 1 | 0.046584081 |
| | | [Source: HGNC Symbol; Acc: HGNC: 26966] | |
| ENSG00000167815 | PRDX2 | peroxiredoxin 2 | 0.046640005 |
| | | [Source: HGNC Symbol; Acc: HGNC: 9353] | |
| ENSG00000140564 | FURIN | “furin, paired basic amino acid cleaving enzyme | 0.046653045 |
| | | [Source: HGNC Symbol; Acc: HGNC: 8568]” | |
| ENSG00000059122 | FLYWCH1 | FLYWCH-type zinc finger 1 | 0.046934685 |
| | | [Source: HGNC Symbol; Acc: HGNC: 25404] | |
| ENSG00000070423 | RNF126 | ring finger protein 126 | 0.047070015 |
| | | [Source: HGNC Symbol; Acc: HGNC: 21151] | |
| ENSG00000027847 | B4GALT7 | “beta-1,4-galactosyltransferase 7 | 0.047130774 |
| | | [Source: HGNC Symbol; Acc: HGNC: 930]” | |
| ENSG00000108518 | PFN1 | profilin 1 | 0.047157356 |
| | | [Source: HGNC Symbol; Acc: HGNC: 8881] | |
| ENSG00000110711 | AIP | aryl hydrocarbon receptor interacting protein | 0.04721490 |
| | | [Source: HGNC Symbol; Acc: HGNC: 358] | |
| ENSG00000164068 | RNF123 | ring finger protein 123 | 0.047214902 |
| | | [Source: HGNC Symbol; Acc: HGNC: 21148] | |
| ENSG00000137216 | TMEM63B | transmembrane protein 63 B | 0.047244731 |
| | | [Source: HGNC Symbol; Acc: HGNC: 17735] | |
| ENSG00000179588 | ZFPM1 | “zinc finger protein, FOG family member 1 | 0.047461433 |
| | | [Source: HGNC Symbol; Acc: HGNC: 19762]” | |
| ENSG00000188070 | C11orf95 | chromosome 11 open reading frame 95 | 0.047461433 |
| | | [Source: HGNC Symbol; Acc: HGNC: 28449] | |
| ENSG00000162722 | TRIM58 | tripartite motif containing 58 | 0.047475759 |
| | | [Source: HGNC Symbol; Acc: HGNC: 24150] | |
| ENSG00000068724 | TTC7A | tetratricopeptide repeat domain 7A | 0.0476766 |
| | | [Source: HGNC Symbol; Acc: HGNC: 19750] | |
| ENSG00000142227 | EMP3 | epithelial membrane protein 3 | 0.0476766 |
| | | [Source: HGNC Symbol; Acc: HGNC: 3335] | |
| ENSG00000159714 | ZDHHC1 | zinc finger DHHC-type containing 1 | 0.04772167 |
| | | [Source: HGNC Symbol; Acc: HGNC: 17916] | |
| ENSG00000196182 | STK40 | serine/threonine kinase 40 | 0.047779011 |
| | | [Source: HGNC Symbol; Acc: HGNC: 21373] | |
| ENSG00000099875 | MKNK2 | MAP kinase interacting serine/threonine kinase 2 | 0.047824795 |
| | | [Source: HGNC Symbol; Acc: HGNC: 7111] | |
| ENSG00000132514 | CLEC10A | C-type lectin domain containing 10A | 0.047824795 |
| | | [Source: HGNC Symbol; Acc: HGNC: 16916] | |
| ENSG00000197982 | C1orf122 | chromosome 1 open reading frame 122 | 0.047824795 |
| | | [Source: HGNC Symbol; Acc: HGNC: 24789] | |
| ENSG00000204348 | DXO | decapping exoribonuclease | 0.047913021 |
| | | [Source: HGNC Symbol; Acc: HGNC: 2992] | |
| ENSG00000259856 | RAB43P1 | RAB43 pseudogene 1 | 0.048194625 |
| | | [Source: HGNC Symbol; Acc: HGNC: 33153] | |
| ENSG00000103254 | FAM173A | family with sequence similarity 173 member A | 0.048206847 |
| | | [Source: HGNC Symbol; Acc: HGNC: 14152] | |
| ENSG00000196453 | ZNF777 | zinc finger protein 777 | 0.048246269 |
| | | [Source: HGNC Symbol; Acc: HGNC: 22213] | |
| ENSG00000167173 | C15orf39 | chromosome 15 open reading frame 39 | 0.048259142 |
| | | [Source: HGNC Symbol; Acc: HGNC: 24497] | |
| ENSG00000172534 | HCFC1 | host cell factor C1 | 0.04840783 |
| | | [Source: HGNC Symbol; Acc: HGNC: 4839] | |
| ENSG00000198804 | MT-CO1 | mitochondrially encoded cytochrome c oxidase I | 0.048409706 |
| | | [Source: HGNC Symbol; Acc: HGNC: 7419] | |
| ENSG00000244486 | SCARF2 | scavenger receptor class F member 2 | 0.048617065 |
| | | [Source: HGNC Symbol; Acc: HGNC: 19869] | |
| ENSG00000125652 | ALKBH7 | alkB homolog 7 | 0.048746819 |
| | | [Source: HGNC Symbol; Acc: HGNC: 21306] | |
| ENSG00000176101 | SSNA1 | SS nuclear autoantigen 1 | 0.048934402 |
| | | [Source: HGNC Symbol; Acc: HGNC: 11321] | |
| ENSG00000129103 | SUMF2 | sulfatase modifying factor 2 | 0.048950286 |
| | | [Source: HGNC Symbol; Acc: HGNC: 20415] | |
| ENSG00000115756 | HPCAL1 | hippocalcin like 1 | 0.049011448 |
| | | [Source: HGNC Symbol; Acc: HGNC: 5145] | |
| ENSG00000243566 | UPK3B | uroplakin 3B | 0.049012128 |
| | | [Source: HGNC Symbol; Acc: HGNC: 21444] | |
| ENSG00000149150 | SLC43A1 | solute carrier family 43 member 1 | 0.049128197 |
| | | [Source: HGNC Symbol; Acc: HGNC: 9225] | |
| ENSG00000103227 | LMF1 | lipase maturation factor 1 | 0.049193782 |
| | | [Source: HGNC Symbol; Acc: HGNC: 14154] | |
| ENSG00000271959 | | | 0.049311473 |
| ENSG00000261222 | | | 0.049471704 |
| ENSG00000126217 | MCF2L | MCF.2 cell line derived transforming sequence like | 0.049648177 |
| | | [Source: HGNC Symbol; Acc: HGNC: 14576] | |
| ENSG00000104964 | AES | amino-terminal enhancer of split | 0.049726662 |
| | | [Source: HGNC Symbol; Acc: HGNC: 307] | |
| ENSG00000127831 | VIL1 | villin 1 | 0.049726662 |
| | | [Source: HGNC Symbol; Acc: HGNC: 12690] | |
| ENSG00000169738 | DCXR | dicarbonyl and L-xylulose reductase | 0.049760492 |
| | | [Source: HGNC Symbol; Acc: HGNC: 18985] | |
| ENSG00000086015 | MAST2 | microtubule associated serine/threonine kinase 2 | 0.04998097 |
| | | [Source: HGNC Symbol; Acc: HGNC: 19035] | |

TABLE 8
AC3 GENES

| Ensembl | Symbol | Description | Score/AUC |
|---|---|---|---|
| ENSG00000251705 | RNA5-8SP6 | “RNA, 5.8S ribosomal pseudogene 6 | 5.29E−67 |
| | | [Source: HGNC Symbol; Acc: HGNC: 41960]” | |
| ENSG00000133110 | POSTN | periostin | 1.78E−06 |
| | | [Source: HGNC Symbol; Acc: HGNC: 16953] | |
| ENSG00000142449 | FBN3 | fibrillin 3 | 2.85E−05 |
| | | [Source: HGNC Symbol; Acc: HGNC: 18794] | |
| ENSG00000183668 | PSG9 | pregnancy specific beta-1-glycoprotein 9 | 9.26E−05 |
| | | [Source: HGNC Symbol; Acc: HGNC: 9526] | |
| ENSG00000258628 | | | 0.000107247 |
| ENSG00000260290 | | | 0.000163871 |
| ENSG00000224367 | OACYLP | “O-acyltransferase like, pseudogene | 0.000275943 |
| | | [Source: HGNC Symbol; Acc: HGNC: 44362]” | |
| ENSG00000108018 | SORCS1 | sortilin related VPS 10 domain containing receptor 1 | 0.000366056 |
| | | [Source: HGNC Symbol; Acc: HGNC: 16697] | |
| ENSG00000135454 | B4GALNT1 | “beta-1,4-N-acetyl-galactosaminyltransferase 1 | 0.000429017 |
| | | [Source: HGNC Symbol; Acc: HGNC: 4117]” | |
| ENSG00000266172 | NA | NA | 0.000574378 |
| ENSG00000173769 | TOPAZ1 | testis and ovary specific PAZ domain containing 1 | 0.00059023 |
| | | [Source: HGNC Symbol; Acc: HGNC: 24746] | |
| ENSG00000181378 | CFAP65 | cilia and flagella associated protein 65 | 0.000602774 |
| | | [Source: HGNC Symbol; Acc: HGNC: 25325] | |
| ENSG00000174498 | IGDCC3 | immunoglobulin superfamily DCC subclass member 3 | 0.00062895 |
| | | [Source: HGNC Symbol; Acc: HGNC: 9700] | |
| ENSG00000089116 | LHX5 | LIM homeobox 5 | 0.000639272 |
| | | [Source: HGNC Symbol; Acc: HGNC: 14216] | |
| ENSG00000161270 | NPHS1 | “NPHS1, nephrin | 0.000697583 |
| | | [Source: HGNC Symbol; Acc: HGNC: 7908]” | |
| ENSG00000006210 | CX3CL1 | C-X3-C motif chemokine ligand 1 | 0.000730947 |
| | | [Source: HGNC Symbol; Acc: HGNC: 10647] | |
| ENSG00000249618 | LINC02465 | long intergenic non-protein coding RNA 2465 | 0.000730947 |
| | | [Source: HGNC Symbol; Acc: HGNC: 53403] | |
| ENSG00000253871 | | | 0.000789684 |
| ENSG00000130540 | SULT4A1 | sulfotransferase family 4A member 1 | 0.000791019 |
| | | [Source: HGNC Symbol; Acc: HGNC: 14903] | |
| ENSG00000148942 | SLC5A12 | solute carrier family 5 member 12 | 0.000819378 |
| | | [Source: HGNC Symbol; Acc: HGNC: 28750] | |
| ENSG00000226790 | HNRNPA3P1 | heterogeneous nuclear ribonucleoprotein A3 | 0.000826721 |
| | | pseudogene 1 | |
| | | [Source: HGNC Symbol; Acc: HGNC: 13729] | |
| ENSG00000160460 | SPTBN4 | “spectrin beta, non-erythrocytic 4 | 0.000837162 |
| | | [Source: HGNC Symbol; Acc: HGNC: 14896]” | |
| ENSG00000243130 | PSG11 | pregnancy specific beta-1-glycoprotein 11 | 0.000837162 |
| | | [Source: HGNC Symbol; Acc: HGNC: 9516] | |
| ENSG00000244694 | PTCHD4 | patched domain containing 4 | 0.000837162 |
| | | [Source: HGNC Symbol; Acc: HGNC: 21345] | |
| ENSG00000249464 | LINC01091 | long intergenic non-protein coding RNA 1091 | 0.000837162 |
| | | [Source: HGNC Symbol; Acc: HGNC: 27721] | |
| ENSG00000011677 | GABRA3 | gamma-aminobutyric acid type A receptor | 0.000896018 |
| | | alpha3 subunit | |
| | | [Source: HGNC Symbol; Acc: HGNC: 4077] | |
| ENSG00000256343 | | | 0.00097441 |
| ENSG00000039139 | DNAH5 | dynein axonemal heavy chain 5 | 0.001236713 |
| | | [Source: HGNC Symbol; Acc: HGNC: 2950] | |
| ENSG00000130635 | COL5A1 | collagen type V alpha 1 chain | 0.001236713 |
| | | [Source: HGNC Symbol; Acc: HGNC: 2209] | |
| ENSG00000242512 | LINC01206 | long intergenic non-protein coding RNA 1206 | 0.001253551 |
| | | [Source: HGNC Symbol; Acc: HGNC: 49637] | |
| ENSG00000164692 | COL1A2 | collagen type I alpha 2 chain | 0.00126185 |
| | | [Source: HGNC Symbol; Acc: HGNC: 2198] | |
| ENSG00000259841 | LINC01566 | long intergenic non-protein coding RNA 1566 | 0.001274332 |
| | | [Source: HGNC Symbol; Acc: HGNC: 27555] | |
| ENSG00000170777 | TPD52L3 | tumor protein D52 like 3 | 0.001484087 |
| | | [Source: HGNC Symbol; Acc: HGNC: 23382] | |
| ENSG00000186487 | MYT1L | myelin transcription factor 1 like | 0.001774703 |
| | | [Source: HGNC Symbol; Acc: HGNC: 7623] | |
| ENSG00000082175 | PGR | progesterone receptor | 0.001879127 |
| | | [Source: HGNC Symbol; Acc: HGNC: 8910] | |
| ENSG00000205922 | ONECUT3 | one cut homeobox 3 | 0.001993667 |
| | | [Source: HGNC Symbol; Acc: HGNC: 13399] | |
| ENSG00000249341 | | | 0.002126073 |
| ENSG00000139865 | TTC6 | tetratricopeptide repeat domain 6 | 0.002268239 |
| | | [Source: HGNC Symbol; Acc: HGNC: 19739] | |
| ENSG00000154478 | GPR26 | G protein-coupled receptor 26 | 0.002268239 |
| | | [Source: HGNC Symbol; Acc: HGNC: 4481] | |
| ENSG00000174358 | SLC6A19 | solute carrier family 6 member 19 | 0.002268239 |
| | | [Source: HGNC Symbol; Acc: HGNC: 27960] | |
| ENSG00000235711 | ANKRD34C | ankyrin repeat domain 34C | 0.002268239 |
| | | [Source: HGNC Symbol; Acc: HGNC: 33888] | |
| ENSG00000170381 | SEMA3E | semaphorin 3E | 0.002327494 |
| | | [Source: HGNC Symbol; Acc: HGNC: 10727] | |
| ENSG00000142611 | PRDM16 | PR/SET domain 16 | 0.002350766 |
| | | [Source: HGNC Symbol; Acc: HGNC: 14000] | |
| ENSG00000205396 | LINC00661 | long intergenic non-protein coding RNA 661 | 0.002366966 |
| | | [Source: HGNC Symbol; Acc: HGNC: 27002] | |
| ENSG00000253288 | | | 0.00239082 |
| ENSG00000171435 | KSR2 | kinase suppressor of ras 2 | 0.002607962 |
| | | [Source: HGNC Symbol; Acc: HGNC: 18610] | |
| ENSG00000256616 | | | 0.002639596 |
| ENSG00000171804 | WDR87 | WD repeat domain 87 | 0.002806683 |
| | | [Source: HGNC Symbol; Acc: HGNC: 29934] | |
| ENSG00000237125 | HAND2-AS1 | HAND2 antisense RNA 1 (head to head) | 0.00289999 |
| | | [Source: HGNC Symbol; Acc: HGNC: 48872] | |
| ENSG00000240694 | PNMA2 | PNMA family member 2 | 0.002940811 |
| | | [Source: HGNC Symbol; Acc: HGNC: 9159] | |
| ENSG00000102452 | NALCN | sodium leak channel, non-selective | 0.003094645 |
| | | [Source: HGNC Symbol; Acc: HGNC: 19082]” | |
| ENSG00000214929 | SPATA31D1 | SPATA31 subfamily D member 1 | 0.003264148 |
| | | [Source: HGNC Symbol; Acc: HGNC: 37283] | |
| ENSG00000115041 | KCNIP3 | potassium voltage-gated channel interacting protein 3 | 0.00328034 |
| | | [Source: HGNC Symbol; Acc: HGNC: 15523] | |
| ENSG00000185038 | MROH2A | maestro heat like repeat family member 2A | 0.003313878 |
| | | [Source: HGNC Symbol; Acc: HGNC: 27936] | |
| ENSG00000138892 | TTLL8 | tubulin tyrosine ligase like 8 | 0.003497603 |
| | | [Source: HGNC Symbol; Acc: HGNC: 34000] | |
| ENSG00000147573 | TRIM55 | tripartite motif containing 55 | 0.003573936 |
| | | [Source: HGNC Symbol; Acc: HGNC: 14215] | |
| ENSG00000165323 | FAT3 | FAT atypical cadherin 3 | 0.003629544 |
| | | [Source: HGNC Symbol; Acc: HGNC: 23112] | |
| ENSG00000142623 | PADI1 | peptidyl arginine deiminase 1 | 0.003697796 |
| | | [Source: HGNC Symbol; Acc: HGNC: 18367] | |
| ENSG00000146521 | LINC01558 | long intergenic non-protein coding RNA 1558 | 0.003779666 |
| | | [Source: HGNC Symbol; Acc: HGNC: 21235] | |
| ENSG00000125255 | SLC10A2 | solute carrier family 10 member 2 | 0.003857127 |
| | | [Source: HGNC Symbol; Acc: HGNC: 10906] | |
| ENSG00000103855 | CD276 | CD276 molecule | 0.004037668 |
| | | [Source: HGNC Symbol; Acc: HGNC: 19137] | |
| ENSG00000168907 | PLA2G4F | phospholipase A2 group IVF | 0.00413675 |
| | | [Source: HGNC Symbol; Acc: HGNC: 27396] | |
| ENSG00000141668 | CBLN2 | cerebellin 2 precursor | 0.004198501 |
| | | [Source: HGNC Symbol; Acc: HGNC: 1544] | |
| ENSG00000197991 | | | 0.004252179 |
| ENSG00000149633 | KIAA1755 | KIAA1755 | 0.004331797 |
| | | [Source: HGNC Symbol; Acc: HGNC: 29372] | |
| ENSG00000157927 | RADIL | Rap associating with DEL domain | 0.004332718 |
| | | [Source: HGNC Symbol; Acc: HGNC: 22226] | |
| ENSG00000138759 | FRAS1 | Fraser extracellular matrix complex subunit 1 | 0.004539725 |
| | | [Source: HGNC Symbol; Acc: HGNC: 19185] | |
| ENSG00000174963 | ZIC4 | Zic family member 4 | 0.004539725 |
| | | [Source: HGNC Symbol; Acc: HGNC: 20393] | |
| ENSG00000177551 | NHLH2 | nescient helix-loop-helix 2 | 0.004560802 |
| | | [Source: HGNC Symbol; Acc: HGNC: 7818] | |
| ENSG00000250230 | | | 0.004576049 |
| ENSG00000204929 | | | 0.00461274 |
| ENSG00000163975 | MELTF | melanotransferrin | 0.004655716 |
| | | [Source: HGNC Symbol; Acc: HGNC: 7037] | |
| ENSG00000095587 | TLL2 | tolloid like 2 | 0.004686524 |
| | | [Source: HGNC Symbol; Acc: HGNC: 11844] | |
| ENSG00000221826 | PSG3 | pregnancy specific beta-1-glycoprotein 3 | 0.004686524 |
| | | [Source: HGNC Symbol; Acc: HGNC: 9520] | |
| ENSG00000105392 | CRX | cone-rod homeobox | 0.004730282 |
| | | [Source: HGNC Symbol; Acc: HGNC: 2383] | |
| ENSG00000188338 | SLC38A3 | solute carrier family 38 member 3 | 0.004737254 |
| | | [Source: HGNC Symbol; Acc: HGNC: 18044] | |
| ENSG00000167654 | ATCAY | “ATCAY, caytaxin | 0.004891089 |
| | | [Source: HGNC Symbol; Acc: HGNC: 779]” | |
| ENSG00000177511 | ST8SIA3 | “ST8 alpha-N-acetyl-neuraminide | 0.00498476 |
| | | alpha-2,8-sialyltransferase 3 | |
| | | [Source: HGNC Symbol; Acc: HGNC: 14269]” | |
| ENSG00000215895 | | | 0.005091461 |
| ENSG00000124466 | LYPD3 | LY6/PLAUR domain containing 3 | 0.005118791 |
| | | [Source: HGNC Symbol; Acc: HGNC: 24880] | |
| ENSG00000084636 | COL16A1 | collagen type XVI alpha 1 chain | 0.00516436 |
| | | [Source: HGNC Symbol; Acc: HGNC: 2193] | |
| ENSG00000104537 | ANXA13 | annexin A13 | 0.005166641 |
| | | [Source: HGNC Symbol; Acc: HGNC: 536] | |
| ENSG00000145526 | CDH18 | cadherin 18 | 0.005245896 |
| | | [Source: HGNC Symbol; Acc: HGNC: 1757] | |
| ENSG00000161103 | | | 0.005294938 |
| ENSG00000168484 | SFTPC | surfactant protein C | 0.005473496 |
| | | [Source: HGNC Symbol; Acc: HGNC: 10802] | |
| ENSG00000188886 | ASTL | astacin like metalloendopeptidase | 0.005530506 |
| | | [Source: HGNC Symbol; Acc: HGNC: 31704] | |
| ENSG00000198765 | SYCP1 | synaptonemal complex protein 1 | 0.005554714 |
| | | [Source: HGNC Symbol; Acc: HGNC: 11487] | |
| ENSG00000234177 | LINC01114 | long intergenic non-protein coding RNA 1114 | 0.005808942 |
| | | [Source: HGNC Symbol; Acc: HGNC: 49245] | |
| ENSG00000091656 | ZFHX4 | zinc finger homeobox 4 | 0.005809417 |
| | | [Source: HGNC Symbol; Acc: HGNC: 30939] | |
| ENSG00000151572 | ANO4 | anoctamin 4 | 0.005857886 |
| | | [Source: HGNC Symbol; Acc: HGNC: 23837] | |
| ENSG00000178965 | ERICH3 | glutamate rich 3 | 0.005890875 |
| | | [Source: HGNC Symbol; Acc: HGNC: 25346] | |
| ENSG00000248587 | GDNF-AS1 | GDNF antisense RNA 1 (head to head) | 0.005890875 |
| | | [Source: HGNC Symbol; Acc: HGNC: 43592] | |
| ENSG00000144908 | ALDH1L1 | aldehyde dehydrogenase 1 family member L1 | 0.006015103 |
| | | [Source: HGNC Symbol; Acc: HGNC: 3978] | |
| ENSG00000152822 | GRM1 | glutamate metabotropic receptor 1 | 0.006033529 |
| | | [Source: HGNC Symbol; Acc: HGNC: 4593] | |
| ENSG00000138675 | FGF5 | fibroblast growth factor 5 | 0.006101348 |
| | | [Source: HGNC Symbol; Acc: HGNC: 3683] | |
| ENSG00000187772 | LIN28B | lin-28 homolog B | 0.006111478 |
| | | [Source: HGNC Symbol; Acc: HGNC: 32207] | |
| ENSG00000227471 | AKR1B15 | aldo-keto reductase family 1 member B15 | 0.006366933 |
| | | [Source: HGNC Symbol; Acc: HGNC: 37281] | |
| ENSG00000174502 | SLC26A9 | solute carrier family 26 member 9 | 0.006716425 |
| | | [Source: HGNC Symbol; Acc: HGNC: 14469] | |
| ENSG00000078549 | ADCYAP1R1 | ADCYAP receptor type I | 0.006848491 |
| | | [Source: HGNC Symbol; Acc: HGNC: 242] | |
| ENSG00000159650 | UROC1 | urocanate hydratase 1 | 0.006848491 |
| | | [Source: HGNC Symbol; Acc: HGNC: 26444] | |
| ENSG00000217094 | PPIAP31 | peptidylprolyl isomerase A pseudogene 31 | 0.006866623 |
| | | [Source: HGNC Symbol; Acc: HGNC: 44962] | |
| ENSG00000006128 | TAC1 | tachykinin precursor 1 | 0.006929673 |
| | | [Source: HGNC Symbol; Acc: HGNC: 11517] | |
| ENSG00000158077 | NLRP14 | NLR family pyrin domain containing 14 | 0.006952696 |
| | | [Source: HGNC Symbol; Acc: HGNC: 22939] | |
| ENSG00000223414 | LINC00473 | long intergenic non-protein coding RNA 473 | 0.006952696 |
| | | [Source: HGNC Symbol; Acc: HGNC: 21160] | |
| ENSG00000144488 | ESPNL | espin like | 0.007039881 |
| | | [Source: HGNC Symbol; Acc: HGNC: 27937] | |
| ENSG00000144730 | IL17RD | interleukin 17 receptor D | 0.007141116 |
| | | [Source: HGNC Symbol; Acc: HGNC: 17616] | |
| ENSG00000137819 | PAQR5 | progestin and adipoQ receptor family member 5 | 0.007179081 |
| | | [Source: HGNC Symbol; Acc: HGNC: 29645] | |
| ENSG00000162631 | NTNG1 | netrin G1 | 0.007255358 |
| | | [Source: HGNC Symbol; Acc: HGNC: 23319] | |
| ENSG00000185974 | GRK1 | G protein-coupled receptor kinase 1 | 0.007327997 |
| | | [Source: HGNC Symbol; Acc: HGNC: 10013] | |
| ENSG00000261275 | | | 0.007327997 |
| ENSG00000249267 | LINC00939 | long intergenic non-protein coding RNA 939 | 0.007349367 |
| | | [Source: HGNC Symbol; Acc: HGNC: 48631] | |
| ENSG00000227827 | | | 0.007403828 |
| ENSG00000100065 | CARD10 | caspase recruitment domain family member 10 | 0.007527421 |
| | | [Source: HGNC Symbol; Acc: HGNC: 16422] | |
| ENSG00000119125 | GDA | guanine deaminase | 0.007619923 |
| | | [Source: HGNC Symbol; Acc: HGNC: 4212] | |
| ENSG00000106304 | SPAM1 | sperm adhesion molecule 1 | 0.00781679 |
| | | [Source: HGNC Symbol; Acc: HGNC: 11217] | |
| ENSG00000250493 | | | 0.007835589 |
| ENSG00000158258 | CLSTN2 | calsyntenin 2 | 0.008149414 |
| | | [Source: HGNC Symbol; Acc: HGNC: 17448] | |
| ENSG00000175329 | ISX | intestine specific homeobox | 0.008233566 |
| | | [Source: HGNC Symbol; Acc: HGNC: 28084] | |
| ENSG00000188488 | SERPINA5 | serpin family A member 5 | 0.008534971 |
| | | [Source: HGNC Symbol; Acc: HGNC: 8723] | |
| ENSG00000249584 | LINC02225 | long intergenic non-protein coding RNA 2225 | 0.00866593 |
| | | Source: HGNC Symbol; Acc: HGNC: 53094] | |
| ENSG00000147655 | RSPO2 | R-spondin 2 | 0.008823914 |
| | | [Source: HGNC Symbol; Acc: HGNC: 28583] | |
| ENSG00000171587 | DSCAM | DS cell adhesion molecule | 0.008841283 |
| | | [Source: HGNC Symbol; Acc: HGNC: 3039] | |
| ENSG00000120738 | EGR1 | early growth response 1 | 0.008960131 |
| | | [Source: HGNC Symbol; Acc: HGNC: 3238] | |
| ENSG00000127129 | EDN2 | endothelin 2 | 0.009244272 |
| | | [Source: HGNC Symbol; Acc: HGNC: 3177] | |
| ENSG00000157423 | HYDIN | “HYDIN, axonemal central pair apparatus protein | 0.009244272 |
| | | [Source: HGNC Symbol; Acc: HGNC: 19368]” | |
| ENSG00000196565 | HBG2 | hemoglobin subunit gamma 2 | 0.009327518 |
| | | [Source: HGNC Symbol; Acc: HGNC: 4832] | |
| ENSG00000235881 | | | 0.009327518 |
| ENSG00000111262 | KCNA1 | potassium voltage-gated channel subfamily A member 1 | 0.009418575 |
| | | [Source: HGNC Symbol; Acc: HGNC: 6218] | |
| ENSG00000187527 | ATP13A5 | ATPase 13A5 | 0.009514824 |
| | | [Source: HGNC Symbol; Acc: HGNC: 31789] | |
| ENSG00000188803 | SHISA6 | shisa family member 6 | 0.009514824 |
| | | [Source: HGNC Symbol; Acc: HGNC: 34491] | |
| ENSG00000175535 | PNLIP | pancreatic lipase | 0.009619326 |
| | | [Source: HGNC Symbol; Acc: HGNC: 9155] | |
| ENSG00000225953 | SATB2-AS1 | SATB2 antisense RNA 1 | 0.009647997 |
| | | [Source: HGNC Symbol; Acc: HGNC: 26490] | |
| ENSG00000136695 | IL36RN | interleukin 36 receptor antagonist | 0.009810065 |
| | | [Source: HGNC Symbol; Acc: HGNC: 15561] | |
| ENSG00000259790 | ANP32BP1 | acidic nuclear phosphoprotein 32 family member | 0.009820284 |
| | | B pseudogene 1 | |
| | | [Source: HGNC Symbol; Acc: HGNC: 24267] | |
| ENSG00000225813 | | | 0.009894409 |
| ENSG00000179008 | C14orf39 | chromosome 14 open reading frame 39 | 0.009896903 |
| | | [Source: HGNC Symbol; Acc: HGNC: 19849] | |
| ENSG00000150893 | FREM2 | FRAS1 related extracellular matrix protein 2 | 0.009945757 |
| | | [Source: HGNC Symbol; Acc: HGNC: 25396] | |
| ENSG00000197079 | KRT35 | keratin 35 | 0.009945757 |
| | | [Source: HGNC Symbol; Acc: HGNC: 6453] | |
| ENSG00000231131 | LINC01468 | long intergenic non-protein coding RNA 1468 | 0.010123625 |
| | | [Source: HGNC Symbol; Acc: HGNC: 50913] | |
| ENSG00000268388 | FENDRR | FOXF1 adjacent non-coding developmental | 0.010151023 |
| | | regulatory RNA | |
| | | [Source: HGNC Symbol; Acc: HGNC: 43894] | |
| ENSG00000159251 | ACTC1 | “actin, alpha, cardiac muscle 1 | 0.010212535 |
| | | [Source: HGNC Symbol; Acc: HGNC: 143]” | |
| ENSG00000158125 | XDH | xanthine dehydrogenase | 0.010258334 |
| | | [Source: HGNC Symbol; Acc: HGNC: 12805] | |
| ENSG00000156222 | SLC28A1 | solute carrier family 28 member 1 | 0.010392835 |
| | | [Source: HGNC Symbol; Acc: HGNC: 11001] | |
| ENSG00000260759 | | | 0.010741484 |
| ENSG00000110975 | SYT10 | synaptotagmin 10 | 0.010787993 |
| | | [Source: HGNC Symbol; Acc: HGNC: 19266] | |
| ENSG00000186185 | KIF18B | kinesin family member 18B | 0.010844893 |
| | | [Source: HGNC Symbol; Acc: HGNC: 27102] | |
| ENSG00000110887 | DAO | D-amino acid oxidase | 0.011064297 |
| | | [Source: HGNC Symbol; Acc: HGNC: 2671] | |
| ENSG00000132297 | HHLA1 | HERV-H LTR-associating 1 | 0.011064297 |
| | | [Source: HGNC Symbol; Acc: HGNC: 4904] | |
| ENSG00000146839 | ZAN | zonadhesin (gene/pseudogene) | 0.011064297 |
| | | [Source: HGNC Symbol; Acc: HGNC: 12857] | |
| ENSG00000215864 | NBPF7 | NBPF member 7 | 0.01113278 |
| | | [Source: HGNC Symbol; Acc: HGNC: 31989] | |
| ENSG00000233395 | LINC00841 | long intergenic non-protein coding RNA 841 | 0.011213055 |
| | | [Source: HGNC Symbol; Acc: HGNC: 27430] | |
| ENSG00000177354 | C10orf71 | chromosome 10 open reading frame 71 | 0.011268372 |
| | | [Source: HGNC Symbol; Acc: HGNC: 26973] | |
| ENSG00000148357 | HMCN2 | hemicentin 2 | 0.01150342 |
| | | [Source: HGNC Symbol; Acc: HGNC: 21293] | |
| ENSG00000215405 | NA | NA | 0.011599767 |
| ENSG00000203900 | | | 0.011732314 |
| ENSG00000218672 | | | 0.011732314 |
| ENSG00000261104 | | | 0.011732314 |
| ENSG00000123243 | ITIH5 | inter-alpha-trypsin inhibitor heavy chain family | 0.011851589 |
| | | member 5 | |
| | | [Source: HGNC Symbol; Acc: HGNC: 21449] | |
| ENSG00000213467 | HMGB1P37 | high mobility group box 1 pseudogene 37 | 0.011876979 |
| | | [Source: HGNC Symbol; Acc: HGNC: 39184] | |
| ENSG00000119283 | TRIM67 | tripartite motif containing 67 | 0.011900585 |
| | | [Source: HGNC Symbol; Acc: HGNC: 31859] | |
| ENSG00000166984 | TCP10L2 | t-complex 10 like 2 | 0.012009181 |
| | | [Source: HGNC Symbol; Acc: HGNC: 21254] | |
| ENSG00000204941 | PSG5 | pregnancy specific beta-1-glycoprotein 5 | 0.012022323 |
| | | [Source: HGNC Symbol; Acc: HGNC: 9522] | |
| ENSG00000230552 | | | 0.012137266 |
| ENSG00000115155 | OTOF | otoferlin | 0.012228668 |
| | | [Source: HGNC Symbol; Acc: HGNC: 8515] | |
| ENSG00000163395 | IGFN1 | immunoglobulin-like and fibronectin type III | 0.012230791 |
| | | domain containing 1 | |
| | | [Source: HGNC Symbol; Acc: HGNC: 24607] | |
| ENSG00000122778 | KIAA1549 | KIAA1549 | 0.012393242 |
| | | [Source: HGNC Symbol; Acc: HGNC: 22219] | |
| ENSG00000169169 | CPT1C | carnitine palmitoyltransferase 1C | 0.012437719 |
| | | [Source: HGNC Symbol; Acc: HGNC: 18540] | |
| ENSG00000160994 | CCDC105 | coiled-coil domain containing 105 | 0.012486932 |
| | | [Source: HGNC Symbol; Acc: HGNC: 26866] | |
| ENSG00000237515 | SHISA9 | shisa family member 9 | 0.012486932 |
| | | [Source: HGNC Symbol; Acc: HGNC: 37231] | |
| ENSG00000105605 | CACNG7 | calcium voltage-gated channel auxiliary subunit | 0.012676606 |
| | | gamma 7 | |
| | | [Source: HGNC Symbol; Acc: HGNC: 13626] | |
| ENSG00000185739 | SRL | sarcalumenin | 0.012676606 |
| | | [Source: HGNC Symbol; Acc: HGNC: 11295] | |
| ENSG00000101680 | LAMA1 | laminin subunit alpha 1 | 0.012767139 |
| | | [Source: HGNC Symbol; Acc: HGNC: 6481] | |
| ENSG00000240021 | TEX35 | testis expressed 35 | 0.012795538 |
| | | [Source: HGNC Symbol; Acc: HGNC: 25366] | |
| ENSG00000250423 | KIAA1210 | KIAA1210 | 0.012935817 |
| | | [Source: HGNC Symbol; Acc: HGNC: 29218] | |
| ENSG00000198788 | MUC2 | “mucin 2, oligomeric mucus/gel-forming | 0.012968601 |
| | | [Source: HGNC Symbol; Acc: HGNC: 7512]” | |
| ENSG00000205312 | KRT17P4 | keratin 17 pseudogene 4 | 0.013016002 |
| | | [Source: HGNC Symbol; Acc: HGNC: 50722] | |
| ENSG00000214128 | TMEM213 | transmembrane protein 213 | 0.013016002 |
| | | [Source: HGNC Symbol; Acc: HGNC: 27220] | |
| ENSG00000178568 | ERBB4 | erb-b2 receptor tyrosine kinase 4 | 0.013045986 |
| | | [Source: HGNC Symbol; Acc: HGNC: 3432] | |
| ENSG00000175084 | DES | desmin | 0.013296119 |
| | | [Source: HGNC Symbol; Acc: HGNC: 2770] | |
| ENSG00000078295 | ADCY2 | adenylate cyclase 2 | 0.013400191 |
| | | [Source: HGNC Symbol; Acc: HGNC: 233] | |
| ENSG00000132639 | SNAP25 | synaptosome associated protein 25 | 0.013440348 |
| | | [Source: HGNC Symbol; Acc: HGNC: 11132] | |
| ENSG00000187094 | CCK | cholecystokinin | 0.013447765 |
| | | [Source: HGNC Symbol; Acc: HGNC: 1569] | |
| ENSG00000018625 | ATP1A2 | ATPase Na+/K+ transporting subunit alpha 2 | 0.013509365 |
| | | [Source: HGNC Symbol; Acc: HGNC: 800] | |
| ENSG00000168542 | COL3A1 | collagen type III alpha 1 chain | 0.013843652 |
| | | [Source: HGNC Symbol; Acc: HGNC: 2201] | |
| ENSG00000239921 | LINC01471 | long intergenic non-protein coding RNA 1471 | 0.013848913 |
| | | [Source: HGNC Symbol; Acc: HGNC: 51106] | |
| ENSG00000233183 | | | 0.013964273 |
| ENSG00000167798 | C3P1 | complement component 3 precursor pseudogene | 0.013987555 |
| | | [Source: HGNC Symbol; Acc: HGNC: 34414] | |
| ENSG00000183778 | B3GALT5 | “beta-1,3-galactosyltransferase 5 | 0.01405326 |
| | | [Source: HGNC Symbol; Acc: HGNC: 920]” | |
| ENSG00000168481 | LGI3 | leucine rich repeat LGI family member 3 | 0.014132769 |
| | | [Source: HGNC Symbol; Acc: HGNC: 18711] | |
| ENSG00000227744 | LINC01940 | long intergenic non-protein coding RNA 1940 | 0.014149077 |
| | | [Source: HGNC Symbol; Acc: HGNC: 52763] | |
| ENSG00000138162 | TACC2 | transforming acidic coiled-coil containing protein 2 | 0.014184675 |
| | | [Source: HGNC Symbol; Acc: HGNC: 11523] | |
| ENSG00000250049 | | | 0.014522615 |
| ENSG00000236445 | LINC00608 | long intergenic non-protein coding RNA 608 | 0.014606813 |
| | | Source: HGNC Symbol; Acc: HGNC: 27179] | |
| ENSG00000165966 | PDZRN4 | PDZ domain containing ring finger 4 | 0.014718872 |
| | | [Source: HGNC Symbol; Acc: HGNC: 30552] | |
| ENSG00000169876 | MUC17 | “mucin 17, cell surface associated | 0.014749092 |
| | | [Source: HGNC Symbol; Acc: HGNC: 16800]” | |
| ENSG00000078898 | BPIFB2 | BPI fold containing family B member 2 | 0.014831419 |
| | | [Source: HGNC Symbol; Acc: HGNC: 16177] | |
| ENSG00000130528 | HRC | histidine rich calcium binding protein | 0.014902982 |
| | | [Source: HGNC Symbol; Acc: HGNC: 5178] | |
| ENSG00000111799 | COL12A1 | collagen type XII alpha 1 chain | 0.015069239 |
| | | [Source: HGNC Symbol; Acc: HGNC: 2188] | |
| ENSG00000185303 | SFTPA2 | surfactant protein A2 | 0.015347263 |
| | | [Source: HGNC Symbol; Acc: HGNC: 10799] | |
| ENSG00000146648 | EGFR | epidermal growth factor receptor | 0.015466899 |
| | | [Source: HGNC Symbol; Acc: HGNC: 3236] | |
| ENSG00000205592 | MUC19 | “mucin 19, oligomeric | 0.015539471 |
| | | [Source: HGNC Symbol; Acc: HGNC: 14362]” | |
| ENSG00000198597 | ZNF536 | zinc finger protein 536 | 0.015552452 |
| | | [Source: HGNC Symbol; Acc: HGNC: 29025] | |
| ENSG00000120332 | TNN | tenascin N | 0.015559411 |
| | | [Source: HGNC Symbol; Acc: HGNC: 22942] | |
| ENSG00000197406 | DIO3 | iodothyronine deiodinase 3 | 0.015755832 |
| | | [Source: HGNC Symbol; Acc: HGNC: 2885] | |
| ENSG00000204283 | LINC01973 | long intergenic non-protein coding RNA 1973 | 0.015755832 |
| | | [Source: HGNC Symbol; Acc: HGNC: 52800] | |
| ENSG00000151224 | MAT1A | methionine adenosyltransferase 1A | 0.015829354 |
| | | [Source: HGNC Symbol; Acc: HGNC: 6903] | |
| ENSG00000257008 | GPR142 | G protein-coupled receptor 142 | 0.015942034 |
| | | [Source: HGNC Symbol; Acc: HGNC: 20088] | |
| ENSG00000139220 | PPFIA2 | PTPRF interacting protein alpha 2 | 0.015986308 |
| | | [Source: HGNC Symbol; Acc: HGNC: 9246] | |
| ENSG00000141946 | ZIM3 | zinc finger imprinted 3 | 0.015986308 |
| | | [Source: HGNC Symbol; Acc: HGNC: 16366] | |
| ENSG00000178171 | AMER3 | APC membrane recruitment protein 3 | 0.015986308 |
| | | [Source: HGNC Symbol; Acc: HGNC: 26771] | |
| ENSG00000232756 | | | 0.015986308 |
| ENSG00000130477 | UNC13A | unc-13 homolog A | 0.016007101 |
| | | [Source: HGNC Symbol; Acc: HGNC: 23150] | |
| ENSG00000070886 | EPHA8 | EPH receptor A8 | 0.016008143 |
| | | [Source: HGNC Symbol; Acc: HGNC: 3391] | |
| ENSG00000253301 | LINC01606 | long intergenic non-protein coding RNA 1606 | 0.016008143 |
| | | [Source: HGNC Symbol; Acc: HGNC: 51656] | |
| ENSG00000006788 | MYH13 | myosin heavy chain 13 | 0.01606756 |
| | | [Source: HGNC Symbol; Acc: HGNC: 7571] | |
| ENSG00000183287 | CCBE1 | collagen and calcium binding EGF domains 1 | 0.016243979 |
| | | [Source: HGNC Symbol; Acc: HGNC: 29426] | |
| ENSG00000262691 | | | 0.016243979 |
| ENSG00000125740 | FOSB | “FosB proto-oncogene, AP-1 transcription | 0.016263717 |
| | | factor subunit | |
| | | [Source: HGNC Symbol; Acc: HGNC: 3797]” | |
| ENSG00000133083 | DCLK1 | doublecortin like kinase 1 | 0.01630694 |
| | | [Source: HGNC Symbol; Acc: HGNC: 2700] | |
| ENSG00000144820 | ADGRG7 | adhesion G protein-coupled receptor G7 | 0.01630694 |
| | | [Source: HGNC Symbol; Acc: HGNC: 19241] | |
| ENSG00000178031 | ADAMTSL1 | ADAMTS like 1 | 0.016324743 |
| | | [Source: HGNC Symbol; Acc: HGNC: 14632] | |
| ENSG00000187905 | LRRC74B | leucine rich repeat containing 74B | 0.016416472 |
| | | [Source: HGNC Symbol; Acc: HGNC: 34301] | |
| ENSG00000221878 | PSG7 | pregnancy specific beta-1-glycoprotein 7 | 0.016416472 |
| | | (gene/pseudogene) | |
| | | [Source: HGNC Symbol; Acc: HGNC: 9524] | |
| ENSG00000254101 | LINC02055 | long intergenic non-protein coding RNA 2055 | 0.016416472 |
| | | [Source: HGNC Symbol; Acc: HGNC: 52895] | |
| ENSG00000120251 | GRIA2 | glutamate ionotropic receptor AMPA type subunit 2 | 0.016488676 |
| | | [Source: HGNC Symbol; Acc: HGNC: 4572] | |
| ENSG00000233991 | NA | NA | 0.016488676 |
| ENSG00000214402 | LCNL1 | lipocalin like 1 | 0.016554945 |
| | | [Source: HGNC Symbol; Acc: HGNC: 34436] | |
| ENSG00000224271 | | | 0.016611527 |
| ENSG00000257576 | HSPD1P4 | heat shock protein family D (Hsp60) member | 0.016611527 |
| | | 1 pseudogene 4 | |
| | | [Source: HGNC Symbol; Acc: HGNC: 35146] | |
| ENSG00000228549 | | | 0.016653065 |
| ENSG00000178645 | C10orf53 | chromosome 10 open reading frame 53 | 0.016654478 |
| | | [Source: HGNC Symbol; Acc: HGNC: 27421] | |
| ENSG00000100078 | PLA2G3 | phospholipase A2 group III | 0.016825197 |
| | | [Source: HGNC Symbol; Acc: HGNC: 17934] | |
| ENSG00000154099 | DNAAF1 | dynein axonemal assembly factor 1 | 0.016918546 |
| | | [Source: HGNC Symbol; Acc: HGNC: 30539] | |
| ENSG00000183242 | WT1-AS | WT1 antisense RNA | 0.016918546 |
| | | [Source: HGNC Symbol; Acc: HGNC: 18135] | |
| ENSG00000124253 | PCK1 | phosphoenolpyruvate carboxykinase 1 | 0.016968016 |
| | | [Source: HGNC Symbol; Acc: HGNC: 8724] | |
| ENSG00000183304 | FAM9A | family with sequence similarity 9 member A | 0.016968016 |
| | | [Source: HGNC Symbol; Acc: HGNC: 18403] | |
| ENSG00000210127 | MT-TA | mitochondrially encoded tRNA alanine | 0.016968016 |
| | | [Source: HGNC Symbol; Acc: HGNC: 7475] | |
| ENSG00000258679 | | | 0.016968016 |
| ENSG00000130287 | NCAN | neurocan | 0.016985672 |
| | | [Source: HGNC Symbol; Acc: HGNC: 2465] | |
| ENSG00000088340 | FER1L4 | “fer-1 like family member 4, pseudogene | 0.017112355 |
| | | [Source: HGNC Symbol; Acc: HGNC: 15801]” | |
| ENSG00000196415 | PRTN3 | proteinase 3 | 0.017180036 |
| | | [Source: HGNC Symbol; Acc: HGNC: 9495] | |
| ENSG00000135917 | SLC19A3 | solute carrier family 19 member 3 | 0.017339051 |
| | | [Source: HGNC Symbol; Acc: HGNC: 16266] | |
| ENSG00000233539 | | | 0.017342649 |
| ENSG00000176584 | DMBT1P1 | deleted in malignant brain tumors 1 pseudogene 1 | 0.017392388 |
| | | [Source: HGNC Symbol; Acc: HGNC: 49497] | |
| ENSG00000135097 | MSI1 | musashi RNA binding protein 1 | 0.017394805 |
| | | [Source: HGNC Symbol; Acc: HGNC: 7330] | |
| ENSG00000091128 | LAMB4 | laminin subunit beta 4 | 0.017415673 |
| | | [Source: HGNC Symbol; Acc: HGNC: 6491] | |
| ENSG00000168367 | LINC00917 | long intergenic non-protein coding RNA 917 | 0.017415673 |
| | | [Source: HGNC Symbol; Acc: HGNC: 48607] | |
| ENSG00000224668 | IPO8P1 | importin 8 pseudogene 1 | 0.017704945 |
| | | [Source: HGNC Symbol; Acc: HGNC: 41955] | |
| ENSG00000165757 | JCAD | junctional cadherin 5 associated | 0.017712329 |
| | | [Source: HGNC Symbol; Acc: HGNC: 29283] | |
| ENSG00000166558 | SLC38A8 | solute carrier family 38 member 8 | 0.017722732 |
| | | [Source: HGNC Symbol; Acc: HGNC: 32434] | |
| ENSG00000185467 | KPNA7 | karyopherin subunit alpha 7 | 0.017767827 |
| | | [Source: HGNC Symbol; Acc: HGNC: 21839] | |
| ENSG00000247699 | | | 0.017813935 |
| ENSG00000248975 | | | 0.017824111 |
| ENSG00000179813 | FAM216B | family with sequence similarity 216 member B | 0.01797203 |
| | | [Source: HGNC Symbol; Acc: HGNC: 26883] | |
| ENSG00000188706 | ZDHHC9 | zinc finger DHHC-type containing 9 | 0.018020454 |
| | | [Source: HGNC Symbol; Acc: HGNC: 18475] | |
| ENSG00000135472 | FAIM2 | Fas apoptotic inhibitory molecule 2 | 0.018071818 |
| | | [Source: HGNC Symbol; Acc: HGNC: 17067] | |
| ENSG00000173572 | NLRP13 | NLR family pyrin domain containing 13 | 0.018071818 |
| | | [Source: HGNC Symbol; Acc: HGNC: 22937] | |
| ENSG00000089199 | CHGB | chromogranin B | 0.018179173 |
| | | [Source: HGNC Symbol; Acc: HGNC: 1930] | |
| ENSG00000188112 | C6orf132 | chromosome 6 open reading frame 132 | 0.018577564 |
| | | [Source: HGNC Symbol; Acc: HGNC: 21288] | |
| ENSG00000187068 | C3orf70 | chromosome 3 open reading frame 70 | 0.018587013 |
| | | [Source: HGNC Symbol; Acc: HGNC: 33731] | |
| ENSG00000233973 | LINC01360 | long intergenic non-protein coding RNA 1360 | 0.018588752 |
| | | [Source: HGNC Symbol; Acc: HGNC: 50593] | |
| ENSG00000164265 | SCGB3A2 | secretoglobin family 3 A member 2 | 0.018614288 |
| | | [Source: HGNC Symbol; Acc: HGNC: 18391] | |
| ENSG00000176769 | TCERG1L | transcription elongation regulator 1 like | 0.018783363 |
| | | [Source: HGNC Symbol; Acc: HGNC: 23533] | |
| ENSG00000179709 | NLRP8 | NLR family pyrin domain containing 8 | 0.018812736 |
| | | [Source: HGNC Symbol; Acc: HGNC: 22940] | |
| ENSG00000251557 | HNRNPKP3 | heterogeneous nuclear ribonucleoprotein K | 0.018866754 |
| | | pseudogene 3 | |
| | | [Source: HGNC Symbol; Acc: HGNC: 42376] | |
| ENSG00000149654 | CDH22 | cadherin 22 | 0.018978105 |
| | | [Source: HGNC Symbol; Acc: HGNC: 13251] | |
| ENSG00000170426 | SDR9C7 | short chain dehydrogenase/reductase family | 0.018978105 |
| | | 9C member 7 | |
| | | [Source: HGNC Symbol; Acc: HGNC: 29958] | |
| ENSG00000225637 | | | 0.019088297 |
| ENSG00000142408 | CACNG8 | calcium voltage-gated channel auxiliary subunit | 0.019108077 |
| | | gamma 8 | |
| | | [Source: HGNC Symbol; Acc: HGNC: 13628] | |
| ENSG00000230873 | STMND1 | stathmin domain containing 1 | 0.01920241 |
| | | [Source: HGNC Symbol; Acc: HGNC: 44668] | |
| ENSG00000236404 | VLDLR-AS1 | VLDLR antisense RNA 1 | 0.01920241 |
| | | [Source: HGNC Symbol; Acc: HGNC: 49621] | |
| ENSG00000170927 | PKHD1 | “PKHD1, fibrocystin/polyductin | 0.019345013 |
| | | [Source: HGNC Symbol; Acc: HGNC: 9016]” | |
| ENSG00000237289 | CKMT1B | “creatine kinase, mitochondrial 1B | 0.019345013 |
| | | [Source: HGNC Symbol; Acc: HGNC: 1995]” | |
| ENSG00000229817 | | | 0.019542531 |
| ENSG00000259176 | NA | NA | 0.019829586 |
| ENSG00000124092 | CTCFL | CCCTC-binding factor like | 0.019839425 |
| | | [Source: HGNC Symbol; Acc: HGNC: 16234] | |
| ENSG00000259156 | CHEK2P2 | checkpoint kinase 2 pseudogene 2 | 0.019859771 |
| | | [Source: HGNC Symbol; Acc: HGNC: 43578] | |
| ENSG00000203805 | PLPP4 | phospholipid phosphatase 4 | 0.019917239 |
| | | [Source: HGNC Symbol; Acc: HGNC: 23531] | |
| ENSG00000163914 | RHO | rhodopsin | 0.019927103 |
| | | [Source: HGNC Symbol; Acc: HGNC: 10012] | |
| ENSG00000224435 | NF1P6 | neurofibromin 1 pseudogene 6 | 0.019927103 |
| | | [Source: HGNC Symbol; Acc: HGNC: 7771] | |
| ENSG00000240707 | LINC01168 | long intergenic non-protein coding RNA 1168 | 0.019935944 |
| | | [Source: HGNC Symbol; Acc: HGNC: 49537] | |
| ENSG00000130045 | NXNL2 | nucleoredoxin like 2 | 0.020063533 |
| | | [Source: HGNC Symbol; Acc: HGNC: 30482] | |
| ENSG00000162062 | TEDC2 | tubulin epsilon and delta complex 2 | 0.020213465 |
| | | [Source: HGNC Symbol; Acc: HGNC: 25849] | |
| ENSG00000172752 | COL6A5 | collagen type VI alpha 5 chain | 0.020225168 |
| | | [Source: HGNC Symbol; Acc: HGNC: 26674] | |
| ENSG00000101871 | MID1 | midline 1 | 0.020247513 |
| | | [Source: HGNC Symbol; Acc: HGNC: 7095] | |
| ENSG00000137648 | TMPRSS4 | transmembrane serine protease 4 | 0.020387859 |
| | | [Source: HGNC Symbol; Acc: HGNC: 11878] | |
| ENSG00000166473 | PKD1L2 | polycystin 1 like 2 (gene/pseudogene) | 0.020387859 |
| | | [Source: HGNC Symbol; Acc: HGNC: 21715] | |
| ENSG00000257907 | EEF1A1P17 | eukaryotic translation elongation factor 1 alpha | 0.020486161 |
| | | 1 pseudogene 17 | |
| | | [Source: HGNC Symbol; Acc: HGNC: 37890] | |
| ENSG00000128917 | DLL4 | delta like canonical Notch ligand 4 | 0.020505106 |
| | | [Source: HGNC Symbol; Acc: HGNC: 2910] | |
| ENSG00000259380 | | | 0.020626924 |
| ENSG00000179766 | ATP8B5P | “ATPase phospholipid transporting 8B5, pseudogene | 0.02065324 |
| | | [Source: HGNC Symbol; Acc: HGNC: 27245]” | |
| ENSG00000204624 | DISP3 | dispatched RND transporter family member 3 | 0.02065324 |
| | | [Source: HGNC Symbol; Acc: HGNC: 29251] | |
| ENSG00000163689 | C3orf67 | chromosome 3 open reading frame 67 | 0.020743462 |
| | | [Source: HGNC Symbol; Acc: HGNC: 24763] | |
| ENSG00000132321 | IQCA1 | IQ motif containing with AAA domain 1 | 0.020807093 |
| | | [Source: HGNC Symbol; Acc: HGNC: 26195] | |
| ENSG00000249119 | MTND6P4 | mitochondrially encoded NADH: ubiquinone | 0.020807093 |
| | | oxidoreductase core subunit 6 pseudogene 4 | |
| | | [Source: HGNC Symbol; Acc: HGNC: 39467] | |
| ENSG00000019505 | SYT13 | synaptotagmin 13 | 0.020829887 |
| | | [Source: HGNC Symbol; Acc: HGNC: 14962] | |
| ENSG00000143469 | SYT14 | synaptotagmin 14 | 0.020885035 |
| | | [Source: HGNC Symbol; Acc: HGNC: 23143] | |
| ENSG00000196136 | SERPINA3 | serpin family A member 3 | 0.02099734 |
| | | [Source: HGNC Symbol; Acc: HGNC: 16] | |
| ENSG00000165816 | VWA2 | von Willebrand factor A domain containing 2 | 0.021329095 |
| | | [Source: HGNC Symbol; Acc: HGNC: 24709] | |
| ENSG00000183317 | EPHA10 | EPH receptor A10 | 0.021329095 |
| | | [Source: HGNC Symbol; Acc: HGNC: 19987] | |
| ENSG00000072041 | SLC6A15 | solute carrier family 6 member 15 | 0.021369466 |
| | | [Source: HGNC Symbol; Acc: HGNC: 13621] | |
| ENSG00000009709 | PAX7 | paired box 7 | 0.021525887 |
| | | [Source: HGNC Symbol; Acc: HGNC: 8621] | |
| ENSG00000172350 | ABCG4 | ATP binding cassette subfamily G member 4 | 0.021525887 |
| | | [Source: HGNC Symbol; Acc: HGNC: 13884] | |
| ENSG00000183876 | ARSI | arylsulfatase family member I | 0.021711339 |
| | | [Source: HGNC Symbol; Acc: HGNC: 32521] | |
| ENSG00000213934 | HBG1 | hemoglobin subunit gamma 1 | 0.02185053 |
| | | [Source: HGNC Symbol; Acc: HGNC: 4831] | |
| ENSG00000186526 | CYP4F8 | cytochrome P450 family 4 subfamily F member 8 | 0.021913633 |
| | | [Source: HGNC Symbol; Acc: HGNC: 2648] | |
| ENSG00000161940 | BCL6B | B cell CLL/lymphoma 6B | 0.021959359 |
| | | [Source: HGNC Symbol; Acc: HGNC: 1002] | |
| ENSG00000164093 | PITX2 | paired like homeodomain 2 | 0.02227554 |
| | | [Source: HGNC Symbol; Acc: HGNC: 9005] | |
| ENSG00000110786 | PTPN5 | “protein tyrosine phosphatase, non-receptor type 5 | 0.022304898 |
| | | [Source: HGNC Symbol; Acc: HGNC: 9657]” | |
| ENSG00000145642 | SHISAL2B | shisa like 2B | 0.022689972 |
| | | [Source: HGNC Symbol; Acc: HGNC: 34236] | |
| ENSG00000260411 | NA | NA | 0.022689972 |
| ENSG00000135409 | AMHR2 | anti-Mullerian hormone receptor type 2 | 0.022721606 |
| | | [Source: HGNC Symbol; Acc: HGNC: 465] | |
| ENSG00000259458 | | | 0.022776502 |
| ENSG00000068078 | FGFR3 | fibroblast growth factor receptor 3 | 0.022896021 |
| | | [Source: HGNC Symbol; Acc: HGNC: 3690] | |
| ENSG00000161243 | FBXO27 | F-box protein 27 | 0.023440646 |
| | | [Source: HGNC Symbol; Acc: HGNC: 18753] | |
| ENSG00000101004 | NENL | ninein like | 0.023637109 |
| | | [Source: HGNC Symbol; Acc: HGNC: 29163] | |
| ENSG00000121207 | LRAT | lecithin retinol acyltransferase | 0.023637109 |
| | | [Source: HGNC Symbol; Acc: HGNC: 6685] | |
| ENSG00000140527 | WDR93 | WD repeat domain 93 | 0.023652058 |
| | | [Source: HGNC Symbol; Acc: HGNC: 26924] | |
| ENSG00000236824 | BCYRN1 | brain cytoplasmic RNA 1 | 0.023652058 |
| | | [Source: HGNC Symbol; Acc: HGNC: 1022] | |
| ENSG00000101203 | COL20A1 | collagen type XX alpha 1 chain | 0.023671204 |
| | | [Source: HGNC Symbol; Acc: HGNC: 14670] | |
| ENSG00000233977 | | | 0.023671204 |
| ENSG00000148408 | CACNA1B | calcium voltage-gated channel subunit alpha1 B | 0.02389629 |
| | | [Source: HGNC Symbol; Acc: HGNC: 1389] | |
| ENSG00000134240 | EEMGCS2 | 3-hydroxy-3-methylglutaryl-CoA synthase 2 | 0.023926586 |
| | | [Source: HGNC Symbol; Acc: HGNC: 5008] | |
| ENSG00000112186 | CAP2 | cyclase associated actin cytoskeleton regulatory | 0.024121501 |
| | | protein 2 | |
| | | [Source: HGNC Symbol; Acc: HGNC: 20039] | |
| ENSG00000182256 | GABRG3 | gamma-aminobutyric acid type A receptor | 0.024155164 |
| | | gamma3 subunit | |
| | | [Source: HGNC Symbol; Acc: HGNC: 4088] | |
| ENSG00000166159 | LRTM2 | leucine rich repeats and transmembrane domains 2 | 0.024214399 |
| | | [Source: HGNC Symbol; Acc: HGNC: 32443] | |
| ENSG00000132972 | RNF17 | ring finger protein 17 | 0.024283188 |
| | | [Source: HGNC Symbol; Acc: HGNC: 10060] | |
| ENSG00000156076 | WIF1 | WNT inhibitory factor 1 | 0.024283188 |
| | | [Source: HGNC Symbol; Acc: HGNC: 18081] | |
| ENSG00000261649 | GOLGA6L7 | golgin A6 family like 7 | 0.024421599 |
| | | [Source: HGNC Symbol; Acc: HGNC: 37442] | |
| ENSG00000112238 | PRDM13 | PR/SET domain 13 | 0.02443315 |
| | | [Source: HGNC Symbol; Acc: HGNC: 13998] | |
| ENSG00000166391 | MOGAT2 | monoacylglycerol O-acyltransferase 2 | 0.024463522 |
| | | [Source: HGNC Symbol; Acc: HGNC: 23248] | |
| ENSG00000166869 | CHP2 | calcineurin like EF-hand protein 2 | 0.024463522 |
| | | [Source: HGNC Symbol; Acc: HGNC: 24927] | |
| ENSG00000218823 | PAPOLB | poly(A) polymerase beta | 0.024463522 |
| | | [Source: HGNC Symbol; Acc: HGNC: 15970] | |
| ENSG00000265041 | | | 0.024463522 |
| ENSG00000133124 | IRS4 | insulin receptor substrate 4 | 0.024526611 |
| | | [Source: HGNC Symbol; Acc: HGNC: 6128] | |
| ENSG00000118733 | OLFM3 | olfactomedin 3 | 0.024577949 |
| | | [Source: HGNC Symbol; Acc: HGNC: 17990] | |
| ENSG00000196091 | MYBPC1 | “myosin binding protein C, slow type | 0.024577949 |
| | | [Source: HGNC Symbol; Acc: HGNC: 7549]” | |
| ENSG00000105357 | MYH14 | myosin heavy chain 14 | 0.025148395 |
| | | [Source: HGNC Symbol; Acc: HGNC: 23212] | |
| ENSG00000167757 | KLK11 | kallikrein related peptidase 11 | 0.025148395 |
| | | [Source: HGNC Symbol; Acc: HGNC: 6359] | |
| ENSG00000226068 | HNRNPA3P4 | heterogeneous nuclear ribonucleoprotein A3 | 0.025148395 |
| | | pseudogene 4 | |
| | | [Source: HGNC Symbol; Acc: HGNC: 39773] | |
| ENSG00000260072 | | | 0.025148395 |
| ENSG00000130226 | DPP6 | dipeptidyl peptidase like 6 | 0.02515224 |
| | | [Source: HGNC Symbol; Acc: HGNC: 3010] | |
| ENSG00000144648 | ACKR2 | atypical chemokine receptor 2 | 0.02527518 |
| | | [Source: HGNC Symbol; Acc: HGNC: 1565] | |
| ENSG00000169862 | CTNND2 | catenin delta 2 | 0.025318552 |
| | | [Source: HGNC Symbol; Acc: HGNC: 2516] | |
| ENSG00000137766 | UNC13C | unc-13 homolog C | 0.025345898 |
| | | [Source: HGNC Symbol; Acc: HGNC: 23149] | |
| ENSG00000261177 | | | 0.025565995 |
| ENSG00000060656 | PTPRU | “protein tyrosine phosphatase, receptor type U | 0.025652607 |
| | | [Source: HGNC Symbol; Acc: HGNC: 9683]” | |
| ENSG00000260305 | NTRK3-AS1 | NTRK3 antisense RNA 1 | 0.02580767 |
| | | [Source: HGNC Symbol; Acc: HGNC: 27532] | |
| ENSG00000187955 | COL14A1 | collagen type XIV alpha 1 chain | 0.025820696 |
| | | [Source: HGNC Symbol; Acc: HGNC: 2191] | |
| ENSG00000089225 | TBX5 | T-box 5 | 0.025834296 |
| | | [Source: HGNC Symbol; Acc: HGNC: 11604] | |
| ENSG00000224209 | LINC00466 | long intergenic non-protein coding RNA 466 | 0.025987072 |
| | | [Source: HGNC Symbol; Acc: HGNC: 27294] | |
| ENSG00000151474 | FRMD4A | FERM domain containing 4A | 0.026041989 |
| | | [Source: HGNC Symbol; Acc: HGNC: 25491] | |
| ENSG00000039987 | BEST2 | bestrophin 2 | 0.026152714 |
| | | [Source: HGNC Symbol; Acc: HGNC: 17107] | |
| ENSG00000266795 | NA | NA | 0.026198035 |
| ENSG00000181143 | MUC16 | “mucin 16, cell surface associated | 0.026247081 |
| | | [Source: HGNC Symbol; Acc: HGNC: 15582]” | |
| ENSG00000069431 | ABCC9 | ATP binding cassette subfamily C member 9 | 0.026486685 |
| | | [Source: HGNC Symbol; Acc: HGNC: 60] | |
| ENSG00000100312 | ACR | acrosin | 0.02666392 |
| | | [Source: HGNC Symbol; Acc: HGNC: 126] | |
| ENSG00000254042 | | | 0.026721536 |
| ENSG00000180251 | SLC9A4 | solute carrier family 9 member A4 | 0.026759131 |
| | | [Source: HGNC Symbol; Acc: HGNC: 11077] | |
| ENSG00000237390 | | | 0.026759131 |
| ENSG00000246695 | RASSF8-AS1 | RASSF8 antisense RNA 1 | 0.026759131 |
| | | [Source: HGNC Symbol; Acc: HGNC: 48637] | |
| ENSG00000256612 | CYP2B7P | “cytochrome P450 family 2 subfamily B member | 0.026759131 |
| | | 7, pseudogene | |
| | | [Source: HGNC Symbol; Acc: HGNC: 2616]” | |
| ENSG00000165973 | NELL1 | neural EGFL like 1 | 0.026774963 |
| | | [Source: HGNC Symbol; Acc: HGNC: 7750] | |
| ENSG00000172900 | | | 0.02698221 |
| ENSG00000149926 | FAM57B | family with sequence similarity 57 member B | 0.02707614 |
| | | [Source: HGNC Symbol; Acc: HGNC: 25295] | |
| ENSG00000107295 | SH3GL2 | “SH3 domain containing GRB2 like 2, endophilin A1 | 0.027164347 |
| | | [Source: HGNC Symbol; Acc: HGNC: 10831]” | |
| ENSG00000173227 | SYT12 | synaptotagmin 12 | 0.027164347 |
| | | [Source: HGNC Symbol; Acc: HGNC: 18381] | |
| ENSG00000173013 | CCDC96 | coiled-coil domain containing 96 | 0.027208218 |
| | | [Source: HGNC Symbol; Acc: HGNC: 26900] | |
| ENSG00000268460 | | | 0.02723306 |
| ENSG00000234512 | TLR12P | “toll like receptor 12, pseudogene | 0.027406947 |
| | | [Source: HGNC Symbol; Acc: HGNC: 31754]” | |
| ENSG00000135931 | ARMC9 | armadillo repeat containing 9 | 0.02759323 |
| | | [Source: HGNC Symbol; Acc: HGNC: 20730] | |
| ENSG00000148702 | HABP2 | hyaluronan binding protein 2 | 0.027601758 |
| | | [Source: HGNC Symbol; Acc: HGNC: 4798] | |
| ENSG00000136535 | TBR1 | “T-box, brain 1 | 0.028071412 |
| | | [Source: HGNC Symbol; Acc: HGNC: 11590]” | |
| ENSG00000122121 | XPNPEP2 | X-prolyl aminopeptidase 2 | 0.028133383 |
| | | [Source: HGNC Symbol; Acc: HGNC: 12823] | |
| ENSG00000170442 | KRT86 | keratin 86 | 0.028133383 |
| | | [Source: HGNC Symbol; Acc: HGNC: 6463] | |
| ENSG00000197408 | CYP2B6 | cytochrome P450 family 2 subfamily B member 6 | 0.028133383 |
| | | [Source: HGNC Symbol; Acc: HGNC: 2615] | |
| ENSG00000107807 | TLX1 | T cell leukemia homeobox 1 | 0.028207054 |
| | | [Source: HGNC Symbol; Acc: HGNC: 5056] | |
| ENSG00000164694 | FNDC1 | fibronectin type III domain containing 1 | 0.028207054 |
| | | [Source: HGNC Symbol; Acc: HGNC: 21184] | |
| ENSG00000185313 | SCN10A | sodium voltage-gated channel alpha subunit 10 | 0.028207054 |
| | | [Source: HGNC Symbol; Acc: HGNC: 10582] | |
| ENSG00000164107 | HAND2 | heart and neural crest derivatives expressed 2 | 0.028335907 |
| | | [Source: HGNC Symbol; Acc: HGNC: 4808] | |
| ENSG00000133454 | MYO18B | myosin XVIIIB | 0.028417113 |
| | | [Source: HGNC Symbol; Acc: HGNC: 18150] | |
| ENSG00000167723 | TRPV3 | transient receptor potential cation channel | 0.028422765 |
| | | subfamily V member 3 | |
| | | [Source: HGNC Symbol; Acc: HGNC: 18084] | |
| ENSG00000184012 | TMPRSS2 | transmembrane serine protease 2 | 0.028422765 |
| | | [Source: HGNC Symbol; Acc: HGNC: 11876] | |
| ENSG00000233485 | | | 0.028645846 |
| ENSG00000261466 | | | 0.028645846 |
| ENSG00000119547 | ONECUT2 | one cut homeobox 2 | 0.028669408 |
| | | [Source: HGNC Symbol; Acc: HGNC: 8139] | |
| ENSG00000237222 | LINC01968 | long intergenic non-protein coding RNA 1968 | 0.028800664 |
| | | [Source: HGNC Symbol; Acc: HGNC: 52794] | |
| ENSG00000137573 | SULF1 | sulfatase 1 | 0.028919683 |
| | | [Source: HGNC Symbol; Acc: HGNC: 20391] | |
| ENSG00000161609 | CCDC155 | coiled-coil domain containing 155 | 0.028967146 |
| | | [Source: HGNC Symbol; Acc: HGNC: 26520] | |
| ENSG00000250546 | | | 0.028987628 |
| ENSG00000226057 | PHF2P2 | PHD finger protein 2 pseudogene 2 | 0.029139308 |
| | | [Source: HGNC Symbol; Acc: HGNC: 38808] | |
| ENSG00000177045 | SIX5 | SIX homeobox 5 | 0.029298722 |
| | | [Source: HGNC Symbol; Acc: HGNC: 10891] | |
| ENSG00000124440 | HIF3A | hypoxia inducible factor 3 alpha subunit | 0.029322985 |
| | | [Source: HGNC Symbol; Acc: HGNC: 15825] | |
| ENSG00000234828 | IQCM | IQ motif containing M | 0.029461236 |
| | | [Source: HGNC Symbol; Acc: HGNC: 53443] | |
| ENSG00000116721 | PRAMEF1 | PRAME family member 1 | 0.029473652 |
| | | [Source: HGNC Symbol; Acc: HGNC: 28840] | |
| ENSG00000238116 | | | 0.029473652 |
| ENSG00000106689 | LHX2 | LIM homeobox 2 | 0.029512187 |
| | | [Source: HGNC Symbol; Acc: HGNC: 6594] | |
| ENSG00000169344 | UMOD | uromodulin | 0.02959944 |
| | | [Source: HGNC Symbol; Acc: HGNC: 12559] | |
| ENSG00000174279 | EVX2 | even-skipped homeobox 2 | 0.029661965 |
| | | [Source: HGNC Symbol; Acc: HGNC: 3507] | |
| ENSG00000128573 | FOXP2 | forkhead box P2 | 0.029779428 |
| | | [Source: HGNC Symbol; Acc: HGNC: 13875] | |
| ENSG00000251596 | HADHAP1 | “hydroxyacyl-CoA dehydrogenase/3-ketoacyl-CoA | 0.029954508 |
| | | thiolase/enoyl-CoA hydratase (trifunctional protein), | |
| | | alpha subunit pseudogene 1 | |
| | | [Source: HGNC Symbol; Acc: HGNC: 4802]” | |
| ENSG00000002746 | HECW1 | “HECT, C2 and WW domain containing E3 | 0.029980732 |
| | | ubiquitin protein ligase 1 | |
| | | [Source: HGNC Symbol; Acc: HGNC: 22195]” | |
| ENSG00000081248 | CACNA1S | calcium voltage-gated channel subunit alpha1 S | 0.029997906 |
| | | [Source: HGNC Symbol; Acc: HGNC: 1397] | |
| ENSG00000166596 | CFAP52 | cilia and flagella associated protein 52 | 0.030027148 |
| | | [Source: HGNC Symbol; Acc: HGNC: 16053] | |
| ENSG00000205176 | REXO1L1P | “REXO1 like 1, pseudogene | 0.030066221 |
| | | [Source: HGNC Symbol; Acc: HGNC: 24660]” | |
| ENSG00000152910 | CNTNAP4 | contactin associated protein like 4 | 0.030144659 |
| | | [Source: HGNC Symbol; Acc: HGNC: 18747] | |
| ENSG00000106078 | COBL | cordon-bleu WH2 repeat protein | 0.030263618 |
| | | [Source: HGNC Symbol; Acc: HGNC: 22199] | |
| ENSG00000177103 | DSCAML1 | DS cell adhesion molecule like 1 | 0.030299369 |
| | | [Source: HGNC Symbol; Acc: HGNC: 14656] | |
| ENSG00000131044 | TTLL9 | tubulin tyrosine ligase like 9 | 0.030317293 |
| | | [Source: HGNC Symbol; Acc: HGNC: 16118] | |
| ENSG00000170703 | TTLL6 | tubulin tyrosine ligase like 6 | 0.030472844 |
| | | [Source: HGNC Symbol; Acc: HGNC: 26664] | |
| ENSG00000165379 | LRFN5 | leucine rich repeat and fibronectin type III | 0.030532839 |
| | | domain containing 5 | |
| | | [Source: HGNC Symbol; Acc: HGNC: 20360] | |
| ENSG00000198929 | NOS1AP | nitric oxide synthase 1 adaptor protein | 0.030532839 |
| | | [Source: HGNC Symbol; Acc: HGNC: 16859] | |
| ENSG00000236253 | SLC25A3P1 | solute carrier family 25 member 3 pseudogene 1 | 0.030574302 |
| | | [Source: HGNC Symbol; Acc: HGNC: 26869] | |
| ENSG00000205667 | ARSH | arylsulfatase family member H | 0.030639161 |
| | | [Source: HGNC Symbol; Acc: HGNC: 32488] | |
| ENSG00000226440 | | | 0.030639161 |
| ENSG00000131183 | SLC34A1 | solute carrier family 34 member 1 | 0.030803937 |
| | | [Source: HGNC Symbol; Acc: HGNC: 11019] | |
| ENSG00000225649 | | | 0.030847037 |
| ENSG00000006283 | CACNA1G | calcium voltage-gated channel subunit alpha1 G | 0.030936635 |
| | | [Source: HGNC Symbol; Acc: HGNC: 1394] | |
| ENSG00000230392 | | | 0.030977697 |
| ENSG00000234130 | | | 0.031067495 |
| ENSG00000095637 | SORBS1 | sorbin and SH3 domain containing 1 | 0.031086939 |
| | | [Source: HGNC Symbol; Acc: HGNC: 14565] | |
| ENSG00000198010 | DLGAP2 | DLG associated protein 2 | 0.031235492 |
| | | [Source: HGNC Symbol; Acc: HGNC: 2906] | |
| ENSG00000102290 | PCDH11X | protocadherin 11 X-linked | 0.031415941 |
| | | [Source: HGNC Symbol; Acc: HGNC: 8656] | |
| ENSG00000260027 | HOXB7 | homeobox B7 | 0.031452441 |
| | | [Source: HGNC Symbol; Acc: HGNC: 5118] | |
| ENSG00000105664 | COMP | cartilage oligomeric matrix protein | 0.031516506 |
| | | [Source: HGNC Symbol; Acc: HGNC: 2227] | |
| ENSG00000006071 | ABCC8 | ATP binding cassette subfamily C member 8 | 0.031578145 |
| | | [Source: HGNC Symbol; Acc: HGNC: 59] | |
| ENSG00000077522 | ACTN2 | actinin alpha 2 | 0.031657927 |
| | | [Source: HGNC Symbol; Acc: HGNC: 164] | |
| ENSG00000248966 | BCLAF1P1 | BCL2 associated transcription factor 1 pseudogene 1 | 0.031733491 |
| | | [Source: HGNC Symbol; Acc: HGNC: 51329] | |
| ENSG00000124749 | COL21A1 | collagen type XXI alpha 1 chain | 0.031814031 |
| | | [Source: HGNC Symbol; Acc: HGNC: 17025] | |
| ENSG00000142675 | CNKSR1 | connector enhancer of kinase suppressor of Ras 1 | 0.031815396 |
| | | [Source: HGNC Symbol; Acc: HGNC: 19700] | |
| ENSG00000116748 | AMPD1 | adenosine monophosphate deaminase 1 | 0.031850457 |
| | | [Source: HGNC Symbol; Acc: HGNC: 468] | |
| ENSG00000181355 | OFCC1 | orofacial cleft 1 candidate 1 | 0.03221007 |
| | | [Source: HGNC Symbol; Acc: HGNC: 21017] | |
| ENSG00000162510 | MATN1 | “matrilin 1, cartilage matrix protein | 0.032424937 |
| | | [Source: HGNC Symbol; Acc: HGNC: 6907]” | |
| ENSG00000232392 | | | 0.032424937 |
| ENSG00000123572 | NRK | Nik related kinase | 0.032537919 |
| | | [Source: HGNC Symbol; Acc: HGNC: 25391] | |
| ENSG00000267324 | | | 0.032537919 |
| ENSG00000196361 | ELAVL3 | ELAV like RNA binding protein 3 | 0.032579843 |
| | | [Source: HGNC Symbol; Acc: HGNC: 3314] | |
| ENSG00000204661 | C5orf60 | chromosome 5 open reading frame 60 | 0.032697662 |
| | | [Source: HGNC Symbol; Acc: HGNC: 27753] | |
| ENSG00000224059 | HSPA8P16 | heat shock protein family A (Hsp70) member | 0.03275969 |
| | | 8 pseudogene 16 | |
| | | [Source: HGNC Symbol; Acc: HGNC: 44931] | |
| ENSG00000114019 | AMOTL2 | angiomotin like 2 | 0.033101928 |
| | | [Source: HGNC Symbol; Acc: HGNC: 17812] | |
| ENSG00000134871 | COL4A2 | collagen type IV alpha 2 chain | 0.033101928 |
| | | [Source: HGNC Symbol; Acc: HGNC: 2203] | |
| ENSG00000162706 | CADM3 | cell adhesion molecule 3 | 0.033101928 |
| | | [Source: HGNC Symbol; Acc: HGNC: 17601] | |
| ENSG00000188782 | CATSPER4 | cation channel sperm associated 4 | 0.033196706 |
| | | [Source: HGNC Symbol; Acc: HGNC: 23220] | |
| ENSG00000147689 | FAM83A | family with sequence similarity 83 member A | 0.033349502 |
| | | [Source: HGNC Symbol; Acc: HGNC: 28210] | |
| ENSG00000079841 | RIMS1 | regulating synaptic membrane exocytosis 1 | 0.033394348 |
| | | [Source: HGNC Symbol; Acc: HGNC: 17282] | |
| ENSG00000103647 | CORO2B | coronin 2B | 0.033419797 |
| | | [Source: HGNC Symbol; Acc: HGNC: 2256] | |
| ENSG00000112499 | SLC22A2 | solute carrier family 22 member 2 | 0.033434322 |
| | | [Source: HGNC Symbol; Acc: HGNC: 10966] | |
| ENSG00000183856 | IQGAP3 | IQ motif containing GTPase activating protein 3 | 0.033434322 |
| | | [Source: HGNC Symbol; Acc: HGNC: 20669] | |
| ENSG00000165300 | SLITRK5 | SLIT and NTRK like family member 5 | 0.033483825 |
| | | [Source: HGNC Symbol; Acc: HGNC: 20295] | |
| ENSG00000229972 | IQCF3 | IQ motif containing F3 | 0.033483825 |
| | | [Source: HGNC Symbol; Acc: HGNC: 31816] | |
| ENSG00000261949 | GFY | golgi associated olfactory signaling regulator | 0.033483825 |
| | | [Source: HGNC Symbol; Acc: HGNC: 44663] | |
| ENSG00000171487 | NLRP5 | NLR family pyrin domain containing 5 | 0.033631095 |
| | | [Source: HGNC Symbol; Acc: HGNC: 21269] | |
| ENSG00000129946 | SHC2 | SHC adaptor protein 2 | 0.033699292 |
| | | [Source: HGNC Symbol; Acc: HGNC: 29869] | |
| ENSG00000117501 | MROH9 | maestro heat like repeat family member 9 | 0.03391477 |
| | | [Source: HGNC Symbol; Acc: HGNC: 26287] | |
| ENSG00000136574 | GATA4 | GATA binding protein 4 | 0.034539616 |
| | | [Source: HGNC Symbol; Acc: HGNC: 4173] | |
| ENSG00000106648 | GALNTL5 | polypeptide N-acetylgalactosaminyltransferase like 5 | 0.034620414 |
| | | Source: HGNC Symbol; Acc: HGNC: 21725] | |
| ENSG00000188086 | PRSS45 | serine protease 45 | 0.034840251 |
| | | [Source: HGNC Symbol; Acc: HGNC: 30717] | |
| ENSG00000234537 | | | 0.034858474 |
| ENSG00000226741 | LINC02554 | long intergenic non-protein coding RNA 2554 | 0.034969314 |
| | | [Source: HGNC Symbol; Acc: HGNC: 53594] | |
| ENSG00000004948 | CALCR | calcitonin receptor | 0.034980702 |
| | | [Source: HGNC Symbol; Acc: HGNC: 1440] | |
| ENSG00000142549 | IGLON5 | IgLON family member 5 | 0.034980702 |
| | | [Source: HGNC Symbol; Acc: HGNC: 34550] | |
| ENSG00000250519 | | | 0.034980702 |
| ENSG00000183908 | LRRC55 | leucine rich repeat containing 55 | 0.035005257 |
| | | [Source: HGNC Symbol; Acc: HGNC: 32324] | |
| ENSG00000253974 | NRG1-IT1 | NRG1 intronic transcript 1 | 0.035154264 |
| | | [Source: HGNC Symbol; Acc: HGNC: 43633] | |
| ENSG00000162738 | VANGL2 | VANGL planar cell polarity protein 2 | 0.035338561 |
| | | [Source: HGNC Symbol; Acc: HGNC: 15511] | |
| ENSG00000115648 | MLPH | melanophilin | 0.03575577 |
| | | [Source: HGNC Symbol; Acc: HGNC: 29643] | |
| ENSG00000187997 | C17orf99 | chromosome 17 open reading frame 99 | 0.03575577 |
| | | [Source: HGNC Symbol; Acc: HGNC: 34490] | |
| ENSG00000140279 | DUOX2 | dual oxidase 2 | 0.035790036 |
| | | [Source: HGNC Symbol; Acc: HGNC: 13273] | |
| ENSG00000168077 | SCARA3 | scavenger receptor class A member 3 | 0.035804565 |
| | | [Source: HGNC Symbol; Acc: HGNC: 19000] | |
| ENSG00000159337 | PLA2G4D | phospholipase A2 group IVD | 0.035823277 |
| | | [Source: HGNC Symbol; Acc: HGNC: 30038] | |
| ENSG00000183580 | FBXL7 | F-box and leucine rich repeat protein 7 | 0.035823277 |
| | | [Source: HGNC Symbol; Acc: HGNC: 13604] | |
| ENSG00000218586 | | | 0.035823277 |
| ENSG00000184809 | B3GALT5-AS1 | B3GALT5 antisense RNA 1 | 0.035845893 |
| | | [Source: HGNC Symbol; Acc: HGNC: 16424] | |
| ENSG00000132975 | GPR12 | G protein-coupled receptor 12 | 0.035938935 |
| | | [Source: HGNC Symbol; Acc: HGNC: 4466] | |
| ENSG00000142910 | TINAGL1 | tubulointerstitial nephritis antigen like 1 | 0.035938935 |
| | | [Source: HGNC Symbol; Acc: HGNC: 19168] | |
| ENSG00000075891 | PAX2 | paired box 2 | 0.035996581 |
| | | [Source: HGNC Symbol; Acc: HGNC: 8616] | |
| ENSG00000186393 | KRT26 | keratin 26 | 0.036025366 |
| | | [Source: HGNC Symbol; Acc: HGNC: 30840] | |
| ENSG00000167779 | IGFBP6 | insulin like growth factor binding protein 6 | 0.036065767 |
| | | [Source: HGNC Symbol; Acc: HGNC: 5475] | |
| ENSG00000232667 | | | 0.036065767 |
| ENSG00000263711 | | | 0.036105875 |
| ENSG00000205054 | LINC01121 | long intergenic non-protein coding RNA 1121 | 0.036340531 |
| | | [Source: HGNC Symbol; Acc: HGNC: 49266] | |
| ENSG00000146950 | SHROOM2 | shroom family member 2 | 0.036393162 |
| | | [Source: HGNC Symbol; Acc: HGNC: 630] | |
| ENSG00000143867 | OSR1 | odd-skipped related transciption factor 1 | 0.036631586 |
| | | [Source: HGNC Symbol; Acc: HGNC: 8111] | |
| ENSG00000205976 | | | 0.037140956 |
| ENSG00000196862 | RGPD4 | RANBP2-like and GRIP domain containing 4 | 0.037154755 |
| | | [Source: HGNC Symbol; Acc: HGNC: 32417] | |
| ENSG00000148513 | ANKRD30A | ankyrin repeat domain 30A | 0.037278195 |
| | | [Source: HGNC Symbol; Acc: HGNC: 17234] | |
| ENSG00000101057 | MYBL2 | MYB proto-oncogene like 2 | 0.037361359 |
| | | [Source: HGNC Symbol; Acc: HGNC: 7548] | |
| ENSG00000139144 | PIK3C2G | phosphatidylinositol-4-phosphate 3-kinase | 0.037546969 |
| | | catalytic subunit type 2 | |
| | | Gamma[Source: HGNC Symbol; Acc: HGNC: 8973] | |
| ENSG00000247213 | LINC01498 | long intergenic non-protein coding RNA 1498 | 0.037546969 |
| | | [Source: HGNC Symbol; Acc: HGNC: 51164] | |
| ENSG00000145242 | EPHA5 | EPH receptor A5 | 0.037630556 |
| | | [Source: HGNC Symbol; Acc: HGNC: 3389] | |
| ENSG00000249215 | NCOA4P4 | nuclear receptor coactivator 4 pseudogene 4 | 0.037740576 |
| | | [Source: HGNC Symbol; Acc: HGNC: 52405] | |
| ENSG00000079112 | CDH17 | cadherin 17 | 0.037745905 |
| | | [Source: HGNC Symbol; Acc: HGNC: 1756] | |
| ENSG00000166118 | SPATA19 | spermatogenesis associated 19 | 0.037802718 |
| | | [Source: HGNC Symbol; Acc: HGNC: 30614] | |
| ENSG00000162006 | MSLNL | mesothelin-like | 0.037970738 |
| | | [Source: HGNC Symbol; Acc: HGNC: 14170] | |
| ENSG00000187123 | LYPD6 | LY6/PLAUR domain containing 6 | 0.037980544 |
| | | [Source: HGNC Symbol; Acc: HGNC: 28751] | |
| ENSG00000104313 | EYA1 | EYA transcriptional coactivator and phosphatase 1 | 0.038059131 |
| | | [Source: HGNC Symbol; Acc: HGNC: 3519] | |
| ENSG00000237250 | | | 0.038171217 |
| ENSG00000105290 | APLP1 | amyloid beta precursor like protein 1 | 0.038576481 |
| | | [Source: HGNC Symbol; Acc: HGNC: 597] | |
| ENSG00000138650 | PCDH10 | protocadherin 10 | 0.038631087 |
| | | [Source: HGNC Symbol; Acc: HGNC: 13404] | |
| ENSG00000198914 | POU3F3 | POU class 3 homeobox 3 | 0.03865691 |
| | | [Source: HGNC Symbol; Acc: HGNC: 9216] | |
| ENSG00000117114 | ADGRL2 | adhesion G protein-coupled receptor L2 | 0.039101789 |
| | | [Source: HGNC Symbol; Acc: HGNC: 18582] | |
| ENSG00000185737 | NRG3 | neuregulin 3 | 0.039101789 |
| | | [Source: HGNC Symbol; Acc: HGNC: 7999] | |
| ENSG00000197085 | NPSR1-AS1 | NPSR1 antisense RNA 1 | 0.039361047 |
| | | [Source: HGNC Symbol; Acc: HGNC: 22128] | |
| ENSG00000230102 | LINC02028 | long intergenic non-protein coding RNA 2028 | 0.039602594 |
| | | [Source: HGNC Symbol; Acc: HGNC: 27718] | |
| ENSG00000241158 | ADAMTS9-AS1 | ADAMTS9 antisense RNA 1 | 0.039646513 |
| | | [Source: HGNC Symbol; Acc: HGNC: 40625] | |
| ENSG00000146005 | PSD2 | pleckstrin and Sec7 domain containing 2 | 0.039852243 |
| | | [Source: HGNC Symbol; Acc: HGNC: 19092] | |
| ENSG00000171533 | MAP6 | microtubule associated protein 6 | 0.040171006 |
| | | [Source: HGNC Symbol; Acc: HGNC: 6868] | |
| ENSG00000164294 | GPX8 | glutathione peroxidase 8 (putative) | 0.040207131 |
| | | [Source: HGNC Symbol; Acc: HGNC: 33100] | |
| ENSG00000054356 | PTPRN | “protein tyrosine phosphatase, receptor type N | 0.040248543 |
| | | [Source: HGNC Symbol; Acc: HGNC: 9676]” | |
| ENSG00000077080 | ACTL6B | actin like 6B | 0.040248543 |
| | | [Source: HGNC Symbol; Acc: HGNC: 160] | |
| ENSG00000141434 | MEP1B | meprin A subunit beta | 0.040590193 |
| | | [Source: HGNC Symbol; Acc: HGNC: 7020] | |
| ENSG00000183067 | IGSF5 | immunoglobulin superfamily member 5 | 0.040590193 |
| | | [Source: HGNC Symbol; Acc: HGNC: 5952] | |
| ENSG00000112337 | SLC17A2 | solute carrier family 17 member 2 | 0.040803316 |
| | | [Source: HGNC Symbol; Acc: HGNC: 10930] | |
| ENSG00000161682 | FAM171A2 | family with sequence similarity 171 member A2 | 0.040923418 |
| | | [Source: HGNC Symbol; Acc: HGNC: 30480] | |
| ENSG00000116833 | NR5A2 | nuclear receptor subfamily 5 group A member 2 | 0.040938395 |
| | | [Source: HGNC Symbol; Acc: HGNC: 7984] | |
| ENSG00000143355 | LHX9 | LIM homeobox 9 | 0.041155655 |
| | | [Source: HGNC Symbol; Acc: HGNC: 14222] | |
| ENSG00000139767 | SRRM4 | serine/arginine repetitive matrix 4 | 0.041207854 |
| | | [Source: HGNC Symbol; Acc: HGNC: 29389] | |
| ENSG00000227036 | LINC00511 | long intergenic non-protein coding RNA 511 | 0.041207854 |
| | | [Source: HGNC Symbol; Acc: HGNC: 43564] | |
| ENSG00000105549 | THEG | theg spermatid protein | 0.041581527 |
| | | [Source: HGNC Symbol; Acc: HGNC: 13706] | |
| ENSG00000104967 | NOVA2 | NOVA alternative splicing regulator 2 | 0.041600384 |
| | | [Source: HGNC Symbol; Acc: HGNC: 7887] | |
| ENSG00000183206 | POTEC | POTE ankyrin domain family member C | 0.041620804 |
| | | [Source: HGNC Symbol; Acc: HGNC: 33894] | |
| ENSG00000184302 | SIX6 | SIX homeobox 6 | 0.041631474 |
| | | [Source: HGNC Symbol; Acc: HGNC: 10892] | |
| ENSG00000245651 | | | 0.041631474 |
| ENSG00000179178 | TMEM125 | transmembrane protein 125 | 0.041791867 |
| | | [Source: HGNC Symbol; Acc: HGNC: 28275] | |
| ENSG00000231422 | LINC01516 | long intergenic non-protein coding RNA 1516 | 0.041868067 |
| | | [Source: HGNC Symbol; Acc: HGNC: 51211] | |
| ENSG00000104435 | STMN2 | stathmin 2 | 0.041944166 |
| | | [Source: HGNC Symbol; Acc: HGNC: 10577] | |
| ENSG00000185069 | KRT76 | keratin 76 | 0.042071807 |
| | | [Source: HGNC Symbol; Acc: HGNC: 24430] | |
| ENSG00000060709 | RIMBP2 | RIMS binding protein 2 | 0.042101327 |
| | | [Source: HGNC Symbol; Acc: HGNC: 30339] | |
| ENSG00000261115 | TMEM178B | transmembrane protein 178B | 0.042193233 |
| | | [Source: HGNC Symbol; Acc: HGNC: 44112] | |
| ENSG00000261623 | LINC02179 | long intergenic non-protein coding RNA 2179 | 0.042224694 |
| | | [Source: HGNC Symbol; Acc: HGNC: 53041] | |
| ENSG00000153165 | RGPD3 | RANBP2-like and GRIP domain containing 3 | 0.042347063 |
| | | [Source: HGNC Symbol; Acc: HGNC: 32416] | |
| ENSG00000253230 | LINC00599 | long intergenic non-protein coding RNA 599 | 0.042450091 |
| | | [Source: HGNC Symbol; Acc: HGNC: 27231] | |
| ENSG00000236078 | LINC01447 | long intergenic non-protein coding RNA 1447 | 0.042463159 |
| | | [Source: HGNC Symbol; Acc: HGNC: 50783] | |
| ENSG00000230133 | LINC01721 | long intergenic non-protein coding RNA 1721 | 0.042512831 |
| | | [Source: HGNC Symbol; Acc: HGNC: 52508] | |
| ENSG00000237636 | ANKRD26P3 | ankyrin repeat domain 26 pseudogene 3 | 0.042582368 |
| | | [Source: HGNC Symbol; Acc: HGNC: 39689] | |
| ENSG00000264954 | PRR29-AS1 | PRR29 antisense RNA 1 | 0.042699245 |
| | | [Source: HGNC Symbol; Acc: HGNC: 51822] | |
| ENSG00000166689 | PLEKHA7 | pleckstrin homology domain containing A7 | 0.04272194 |
| | | [Source: HGNC Symbol; Acc: HGNC: 27049] | |
| ENSG00000173826 | KCNH6 | potassium voltage-gated channel subfamily H | 0.042801591 |
| | | member 6 | |
| | | [Source: HGNC Symbol; Acc: HGNC: 18862] | |
| ENSG00000253864 | NA | NA | 0.042900836 |
| ENSG00000166292 | TMEM100 | transmembrane protein 100 | 0.043052047 |
| | | [Source: HGNC Symbol; Acc: HGNC: 25607] | |
| ENSG00000137203 | TFAP2A | transcription factor AP-2 alpha | 0.043105155 |
| | | [Source: HGNC Symbol; Acc: HGNC: 11742] | |
| ENSG00000165970 | SLC6A5 | solute carrier family 6 member 5 | 0.043105155 |
| | | [Source: HGNC Symbol; Acc: HGNC: 11051] | |
| ENSG00000184908 | CLCNKB | chloride voltage-gated channel Kb | 0.043516345 |
| | | [Source: HGNC Symbol; Acc: HGNC: 2027] | |
| ENSG00000197893 | NRAP | nebulin related anchoring protein | 0.043567821 |
| | | [Source: HGNC Symbol; Acc: HGNC: 7988] | |
| ENSG00000169126 | ARMC4 | armadillo repeat containing 4 | 0.043632647 |
| | | [Source: HGNC Symbol; Acc: HGNC: 25583] | |
| ENSG00000245248 | USP2-AS1 | USP2 antisense RNA 1 (head to head) | 0.043635087 |
| | | [Source: HGNC Symbol; Acc: HGNC: 48673] | |
| ENSG00000242866 | STRC | stereocilin | 0.043658722 |
| | | [Source: HGNC Symbol; Acc: HGNC: 16035] | |
| ENSG00000164393 | ADGRF2 | adhesion G protein-coupled receptor F2 | 0.044026611 |
| | | [Source: HGNC Symbol; Acc: HGNC: 18991] | |
| ENSG00000100033 | PRODH | proline dehydrogenase 1 | 0.044045719 |
| | | [Source: HGNC Symbol; Acc: HGNC: 9453] | |
| ENSG00000136352 | NKX2-1 | NK2 homeobox 1 | 0.044046233 |
| | | [Source: HGNC Symbol; Acc: HGNC: 11825] | |
| ENSG00000165566 | AMER2 | APC membrane recruitment protein 2 | 0.044155809 |
| | | [Source: HGNC Symbol; Acc: HGNC: 26360] | |
| ENSG00000163995 | ABLIM2 | actin binding LIM protein family member 2 | 0.044231879 |
| | | [Source: HGNC Symbol; Acc: HGNC: 19195] | |
| ENSG00000165495 | PKNOX2 | PBX/knotted 1 homeobox 2 | 0.044261202 |
| | | [Source: HGNC Symbol; Acc: HGNC: 16714] | |
| ENSG00000144115 | THNSL2 | threonine synthase like 2 | 0.044590058 |
| | | [Source: HGNC Symbol; Acc: HGNC: 25602] | |
| ENSG00000157214 | STEAP2 | STEAP2 metalloreductase | 0.044717969 |
| | | [Source: HGNC Symbol; Acc: HGNC: 17885] | |
| ENSG00000229240 | LINC00710 | long intergenic non-protein coding RNA 710 | 0.044849493 |
| | | [Source: HGNC Symbol; Acc: HGNC: 27386] | |
| ENSG00000168356 | SCN11A | sodium voltage-gated channel alpha subunit 11 | 0.044881812 |
| | | [Source: HGNC Symbol; Acc: HGNC: 10583] | |
| ENSG00000130508 | PXDN | peroxidasin | 0.044899327 |
| | | [Source: HGNC Symbol; Acc: HGNC: 14966] | |
| ENSG00000166444 | ST5 | suppression of tumorigenicity 5 | 0.044899327 |
| | | [Source: HGNC Symbol; Acc: HGNC: 11350] | |
| ENSG00000140600 | SH3GL3 | “SH3 domain containing GRB2 like 3, endophilin A3 | 0.045237232 |
| | | [Source: HGNC Symbol; Acc: HGNC: 10832]” | |
| ENSG00000214181 | NA | NA | 0.045237232 |
| ENSG00000144681 | STAC | SH3 and cysteine rich domain | 0.045389235 |
| | | [Source: HGNC Symbol; Acc: HGNC: 11353] | |
| ENSG00000166863 | TAC3 | tachykinin 3 | 0.045474144 |
| | | [Source: HGNC Symbol; Acc: HGNC: 11521] | |
| ENSG00000169436 | COL22A1 | collagen type XXII alpha 1 chain | 0.045474144 |
| | | [Source: HGNC Symbol; Acc: HGNC: 22989] | |
| ENSG00000172137 | CALB2 | calbindin 2 | 0.045474144 |
| | | [Source: HGNC Symbol; Acc: HGNC: 1435] | |
| ENSG00000223566 | TNRC18P2 | trinucleotide repeat containing 18 pseudogene 2 | 0.045474144 |
| | | [Source: HGNC Symbol; Acc: HGNC: 34014] | |
| ENSG00000175267 | VWA3A | von Willebrand factor A domain containing 3A | 0.045495146 |
| | | [Source: HGNC Symbol; Acc: HGNC: 27088] | |
| ENSG00000175267 | VWA3A | von Willebrand factor A domain containing 3A | 0.045495146 |
| | | [Source: HGNC Symbol; Acc: HGNC: 27088] | |
| ENSG00000183780 | SLC35F3 | solute carrier family 35 member F3 | 0.045495146 |
| | | [Source: HGNC Symbol; Acc: HGNC: 23616] | |
| ENSG00000228983 | SLC47A1P1 | solute carrier family 47 member 1 pseudogene 1 | 0.045495146 |
| | | [Source: HGNC Symbol; Acc: HGNC: 51849] | |
| ENSG00000269332 | GOLGA2P9 | golgin A2 pseudogene 9 | 0.045495146 |
| | | [Source: HGNC Symbol; Acc: HGNC: 49921] | |
| ENSG00000081800 | SLC13A1 | solute carrier family 13 member 1 | 0.045528908 |
| | | [Source: HGNC Symbol; Acc: HGNC: 10916] | |
| ENSG00000155816 | FMN2 | formin 2 | 0.045528908 |
| | | [Source: HGNC Symbol; Acc: HGNC: 14074] | |
| ENSG00000091137 | SLC26A4 | solute carrier family 26 member 4 | 0.045601783 |
| | | [Source: HGNC Symbol; Acc: HGNC: 8818] | |
| ENSG00000129990 | SYT5 | synaptotagmin 5 | 0.045601783 |
| | | [Source: HGNC Symbol; Acc: HGNC: 11513] | |
| ENSG00000173702 | MUC13 | “mucin 13, cell surface associated | 0.045601783 |
| | | [Source: HGNC Symbol; Acc: HGNC: 7511]” | |
| ENSG00000116176 | TPSG1 | tryptase gamma 1 | 0.045645845 |
| | | [Source: HGNC Symbol; Acc: HGNC: 14134] | |
| ENSG00000250420 | AACSP1 | acetoacetyl-CoA synthetase pseudogene 1 | 0.045645845 |
| | | [Source: HGNC Symbol; Acc: HGNC: 18226] | |
| ENSG00000104055 | TGM5 | transglutaminase 5 | 0.045691236 |
| | | [Source: HGNC Symbol; Acc: HGNC: 11781] | |
| ENSG00000109101 | FOXN1 | forkhead box N1 | 0.045781 |
| | | [Source: HGNC Symbol; Acc: HGNC: 12765] | |
| ENSG00000131386 | GALNT15 | polypeptide N-acetylgalactosaminyltransferase 15 | 0.045798489 |
| | | [Source: HGNC Symbol; Acc: HGNC: 21531] | |
| ENSG00000183016 | NA | NA | 0.045937358 |
| ENSG00000248746 | ACTN3 | actinin alpha 3 (gene/pseudogene) | 0.045937358 |
| | | [Source: HGNC Symbol; Acc: HGNC: 165] | |
| ENSG00000259010 | | | 0.04595318 |
| ENSG00000156687 | UNC5D | unc-5 netrin receptor D | 0.046099925 |
| | | [Source: HGNC Symbol; Acc: HGNC: 18634] | |
| ENSG00000213864 | EEF1B2P2 | eukaryotic translation elongation factor 1 beta | 0.046129239 |
| | | 2 pseudogene 2 | |
| | | [Source: HGNC Symbol; Acc: HGNC: 3209] | |
| ENSG00000143107 | FNDC7 | fibronectin type III domain containing 7 | 0.046229298 |
| | | [Source: HGNC Symbol; Acc: HGNC: 26668] | |
| ENSG00000230615 | | | 0.046269835 |
| ENSG00000184227 | ACOT1 | acyl-CoA thioesterase 1 | 0.046363122 |
| | | [Source: HGNC Symbol; Acc: HGNC: 33128] | |
| ENSG00000118194 | TNNT2 | “troponin T2, cardiac type | 0.046453265 |
| | | [Source: HGNC Symbol; Acc: HGNC: 11949]” | |
| ENSG00000172995 | ARPP21 | cAMP regulated phosphoprotein 21 | 0.046453265 |
| | | [Source: HGNC Symbol; Acc: HGNC: 16968] | |
| ENSG00000156103 | MMP16 | matrix metallopeptidase 16 | 0.04649564 |
| | | [Source: HGNC Symbol; Acc: HGNC: 7162] | |
| ENSG00000164904 | ALDH7A1 | aldehyde dehydrogenase 7 family member A1 | 0.04649564 |
| | | [Source: HGNC Symbol; Acc: HGNC: 877] | |
| ENSG00000224743 | TEX26-AS1 | TEX26 antisense RNA 1 | 0.04649564 |
| | | [Source: HGNC Symbol; Acc: HGNC: 42784] | |
| ENSG00000185823 | NPAP1 | nuclear pore associated protein 1 | 0.04652545 |
| | | [Source: HGNC Symbol; Acc: HGNC: 1190] | |
| ENSG00000018607 | ZNF806 | zinc finger protein 806 | 0.046600673 |
| | | [Source: HGNC Symbol; Acc: HGNC: 33228] | |
| ENSG00000179270 | C2orf71 | chromosome 2 open reading frame 71 | 0.046600673 |
| | | [Source: HGNC Symbol; Acc: HGNC: 34383] | |
| ENSG00000186862 | PDZD7 | PDZ domain containing 7 | 0.046710668 |
| | | [Source: HGNC Symbol; Acc: HGNC: 26257] | |
| ENSG00000227525 | RPL7P6 | ribosomal protein L7 pseudogene 6 | 0.046989116 |
| | | [Source: HGNC Symbol; Acc: HGNC: 32430] | |
| ENSG00000236229 | VEZF1P1 | vascular endothelial zinc finger 1 pseudogene 1 | 0.047105132 |
| | | [Source: HGNC Symbol; Acc: HGNC: 32320] | |
| ENSG00000171564 | FGB | fibrinogen beta chain | 0.047251427 |
| | | [Source: HGNC Symbol; Acc: HGNC: 3662] | |
| ENSG00000257175 | | | 0.047316621 |
| ENSG00000248713 | LOC285556 | “Homo sapiens uncharacterized mRNA. | 0.047420932 |
| | | [Source: RefSeq mRNA; Acc: NM_001354435]” | |
| ENSG00000102287 | GABRE | gamma-aminobutyric acid type A receptor | 0.047603565 |
| | | epsilon subunit | |
| | | [Source: HGNC Symbol; Acc: HGNC: 4085] | |
| ENSG00000150086 | NA | NA | 0.0476766 |
| ENSG00000168959 | GRM5 | glutamate metabotropic receptor 5 | 0.0476766 |
| | | [Source: HGNC Symbol; Acc: HGNC: 4597] | |
| ENSG00000184304 | PRKD1 | protein kinase D1 | 0.0476766 |
| | | [Source: HGNC Symbol; Acc: HGNC: 9407] | |
| ENSG00000204055 | | | 0.047795615 |
| ENSG00000164122 | ASB5 | ankyrin repeat and SOCS box containing 5 | 0.047913021 |
| | | [Source: HGNC Symbol; Acc: HGNC: 17180] | |
| ENSG00000123977 | DAW1 | dynein assembly factor with WD repeats 1 | 0.047973 |
| | | [Source: HGNC Symbol; Acc: HGNC: 26383] | |
| ENSG00000156413 | FUT6 | fucosyltransferase 6 | 0.047988989 |
| | | [Source: HGNC Symbol; Acc: HGNC: 4017] | |
| ENSG00000101276 | SLC52A3 | solute carrier family 52 member 3 | 0.048129781 |
| | | [Source: HGNC Symbol; Acc: HGNC: 16187] | |
| ENSG00000168079 | SCARA5 | scavenger receptor class A member 5 | 0.048129781 |
| | | [Source: HGNC Symbol; Acc: HGNC: 28701] | |
| ENSG00000254561 | | | 0.048129781 |
| ENSG00000223949 | ROR1-AS1 | ROR1 antisense RNA 1 | 0.048194625 |
| | | [Source: HGNC Symbol; Acc: HGNC: 40508] | |
| ENSG00000204335 | SP5 | Sp5 transcription factor | 0.048312303 |
| | | [Source: HGNC Symbol; Acc: HGNC: 14529] | |
| ENSG00000204241 | | | 0.04840783 |
| ENSG00000099625 | CBARP | CACN beta subunit associated regulatory protein | 0.048411296 |
| | | [Source: HGNC Symbol; Acc: HGNC: 28617] | |
| ENSG00000143450 | OAZ3 | ornithine decarboxylase antizyme 3 | 0.048617065 |
| | | [Source: HGNC Symbol; Acc: HGNC: 8097] | |
| ENSG00000015520 | NPC1L1 | NPC1 like intracellular cholesterol transporter 1 | 0.048746819 |
| | | [Source: HGNC Symbol; Acc: HGNC: 7898] | |
| ENSG00000188162 | OTOG | otogelin | 0.048746819 |
| | | [Source: HGNC Symbol; Acc: HGNC: 8516] | |
| ENSG00000125492 | BARHL1 | BarH like homeobox 1 | 0.048820483 |
| | | [Source: HGNC Symbol; Acc: HGNC: 953] | |
| ENSG00000145832 | SLC25A48 | solute carrier family 25 member 48 | 0.048934402 |
| | | [Source: HGNC Symbol; Acc: HGNC: 30451] | |
| ENSG00000185686 | PRAME | preferentially expressed antigen in melanoma | 0.048934402 |
| | | [Source: HGNC Symbol; Acc: HGNC: 9336] | |
| ENSG00000229147 | SMPD4P2 | sphingomyelin phosphodiesterase 4 pseudogene 2 | 0.049012128 |
| | | [Source: HGNC Symbol; Acc: HGNC: 39674] | |

A listing of selected and preferred AC3 RNA markers of impending flare, based on their scores and relevance to sublining fibroblast cells is provided below in Table 9. These markers are particularly selected for determining and predicting impending RA flares. The markers are selected from COL1A2, COL5A1, COL16A1, COL14A1, COL4A2, PXDN, ST5, DCLK1, SCARA5, EGFR, EGR1 and ZFHX4.

The markers include several genes for collagen alpha chain subumits, including COL1A2, COL5A1, COL16A1, COL14A1 and COL4A2, as follows: COL1A2 Collagen alpha-2(1) chain. This gene encodes the alpha 2 (pro-a2(1)) chain component of type I collagen, the fibrillary collagen found in most connective tissues; COL5A1 Collagen Type V Alpha 1 Chain a component of type V collagen, which is a low abundance fibrillar collagen; COL16A1 Collagen alpha-1(XVI) chain. This gene encodes the alpha chain of type XVI collagen, a member of the FACIT collagen family (fibril-associated collagens with interrupted helices). Collage type XVI is a fibril-forming collagen that maintains the integrity of the extracellular matric COL14A1 Collagen alpha-1(XIV) chain is a protein that in humans is encoded by the COL14A1 gene. It likely plays a role in collagen binding and cell-cell adhesion; COL4A2 The COL4A2 gene encodes the alpha-2 chain of type IV collagen. Type IV collagen is associated with laminin, entactin, and heparan sulfate proteoglycans to form the sheetlike basement membranes that separate epithelium from connective tissue.

Additional markers are PXDN, ST5, DCLK1, SCARA5, EGFR, EGR1 and ZFHX4. PXDN (peroxidasin) is a heme-containing peroxidase that is secreted into the extracellular matrix and is involved in extracellular matrix formation. ST5 (Suppression Of Tumorigenicity 5 protein), also called DENN Domain Containing 2B. This gene was identified by its ability to suppress the tumorigenicity of Hela cells in nude mice. The protein encoded by this gene contains a C-terminal region that shares similarity with the Rab 3 family of small GTP binding proteins. This protein preferentially binds to the SH3 domain of c-Abl kinase, and acts as a regulator of MAPK1/ERK2 kinase, which may contribute to its ability to reduce the tumorigenic phenotype in cells. May be involved in cytoskeletal organization and tumorigenicity. DCLK1 (Doublecortin Like Kinase 1) is a microtubule-associated protein kinase—a serine/threonine-protein kinase. Doublecortin Like Kinase 1 has been identified as a tuft cell marker in the small intestine and has been reported to mark tumor stem cells in the intestine and pancreas. SCARA5 (Scavenger receptor class A, member 5) is involved in lineage commitment and differentiation of mesenchymal stem cells to adipocytes. EGFR corresponds to epidermal growth factor receptor and is a cell membrane spanning protein induces cell differentiation and proliferation. Alteration and overexpression associated with various cancers. Numerous EGFR antibodies, including specific neutralizing antibodies, have been developed and are in clinical development or clinical practice for applications in cancer. EGR1 (Early growth response protein 1)—also known as ZNF268 (zinc finger protein 268) or NGFI-A (nerve growth factor-induced protein A). EGR-1 is a mammalian transcription factor. EGR-1 is a mechano-sensitive transcriptional factor that stimulates IGF-1R transcription, resulting in vascular remodeling of vein grafts. Early growth response protein 1 is a transcription factor that is rapidly induced by growth factors, cytokines, and stress signals such as radiation, injury, or mechanical stress. ZFHX4 (Zinc Finger Homeobox 4) Predicted to have RNA polymerase II proximal promoter sequence-specific DNA binding activity. RNA polymerase II specific DNA-binding transcription factor activity.

Notably all of the markers provided in Table 9 are also listed in Table 5 above, which provided transcripts common to synovial sublining fibroblasts and AC3. Table 5 also included the a collagen chain gene COL3A1, COMP, FNDC1, GALNT15, SULF1, GPX8 and IGFBP6. COL3A2 corresponds to collagen Type III alpha 1 chain. COMP (cartilage oligomeric matrix protein) Can mediate the interaction of chondrocytes with the cartilage extracellular matrix and may play a role in the structural integrity of cartilage via its interaction with other extracellular matrix proteins such as the collagens and fibronectin. FNDC1 (fibronectin Type III domain containing 1) has alternative names Activation-associated cDNA protein and expressed in synovial lining protein and is an activator of G-protein signaling. GALNT15 (Polypeptide N-Acetylgalactosaminyltransferase 15) is a membrane-bound polypeptide N-acetylgalactosaminyltransferases that catalyzes the first step in mucin-type O-glycosylation of peptides in the Golgi apparatus. SULF1 (Sulfatase 1) is an extracellular heparan sulfate endosulfatase. The enzyme is secreted through the Golgi and is subsequently localized to the cell surface and selectively removes 6-O-sulfate groups from heparan sulfate chains of heparan sulfate proteoglycans. GPX8 (Glutathione Peroxidase 8) is a protein disulfide isomerase and is involved in cellular response to oxidative stress, reducing H2O2 content and oxidative stress in the ER. IGFBP6 (Insulin-like growth factor-binding protein 6) binds insulin-like growth factor and fibronectin and has been shown to modulate the growth promoting effects of the IGFs on cell culture.

TABLE 9
RNA Markers of Impending Flare

| | Col- | | | | | | pct | |
|---|---|---|---|---|---|---|---|---|
| | umn | | | | | PCT | nonzero | baseMean |
| SYMBOL | 1 | geneset | ENSG | Gene | AUC | nonzero | other | prime |
| COL1A2 | SC- | Fibroblast-CD34+ | ENSG00000164692 | ENSG00000164692.13 | 0.84468179 | 1 | 0.532809984 | 51.509861398 |
| | F1 | sublining (SC-F1) | | | | | | |
| COL1A2 | SC- | Fibroblast-HLA-DRAhi | ENSG00000164692 | ENSG00000164692.13 | 0.921394835 | 1 | 0.509613353 | 51.509861398 |
| | F2 | sublining (SC-F2) | | | | | | |
| COL1A2 | SC- | Fibroblast-DKK3+ | ENSG00000164692 | ENSG00000164692.13 | 0.906702534 | 1 | 0.555704441 | 51.509861398 |
| | F3 | sublining (SC-F3) | | | | | | |
| PXDN | SC- | Fibroblast-HLA-DRAhi | ENSG00000130508 | ENSG00000130508.6 | 0.843306415 | 0.831710709 | 0.16670188 | 24.409015138 |
| | F2 | sublining (SC-F2) | | | | | | |
| PXDN | SC- | Fibroblast-DKK3+ | ENSG00000130508 | ENSG00000130508.6 | 0.836927155 | 0.881578947 | 0.227029096 | 24.409015138 |
| | F3 | sublining (SC-F3) | | | | | | |
| COL5A1 | SC- | Fibroblast-CD34+ | ENSG00000130635 | ENSG00000130635.11 | 0.769189965 | 0.809917355 | 0.263486312 | 24.050387975 |
| | F1 | sublining (SC-F1) | | | | | | |
| COL5A1 | SC- | Fibroblast-HLA-DRAhi | ENSG00000130635 | ENSG00000130635.11 | 0.884892773 | 0.95132128 | 0.214874287 | 24.050387975 |
| | F2 | sublining (SC-F2) | | | | | | |
| COL5A1 | SC- | Fibroblast-DKK3+ | ENSG00000130635 | ENSG00000130635.11 | 0.917280819 | 0.98245614 | 0.282733538 | 24.050387975 |
| | F3 | sublining (SC-F3) | | | | | | |
| ZFHX4 | SC- | Fibroblast-CD34+ | ENSG00000091656 | ENSG00000091656.11 | 0.778380395 | 0.714876033 | 0.207125604 | 23.828346616 |
| | F1 | sublining (SC-F1) | | | | | | |
| ZFHX4 | SC- | Fibroblast-DKK3+ | ENSG00000091656 | ENSG00000091656.11 | 0.789767117 | 0.802631579 | 0.228177642 | 23.828346616 |
| | F3 | sublining (SC-F3) | | | | | | |
| COL16A1 | SC- | Fibroblast-HLA-DRAhi | ENSG00000084636 | ENSG00000084636.12 | 0.87323477 | 0.835883171 | 0.105852525 | 23.789446496 |
| | F2 | sublining (SC-F2) | | | | | | |
| ST5 | SC- | Fibroblast-HLA-DRAhi | ENSG00000166444 | ENSG00000166444.13 | 0.833490889 | 0.816411683 | 0.158673146 | 23.39530374 |
| | F2 | sublining (SC-F2) | | | | | | |
| DCLK1 | SC- | Fibroblast-CD34+ | ENSG00000133083 | ENSG00000133083.10 | 0.786731154 | 0.700413223 | 0.168075684 | 23.123692573 |
| | F1 | sublining (SC-F1) | | | | | | |
| DCLK1 | SC- | Fibroblast-HLA-DRAhi | ENSG00000133083 | ENSG00000133083.10 | 0.831312828 | 0.799721836 | 0.126558208 | 23.123692573 |
| | F2 | sublining (SC-F2) | | | | | | |
| EGR1 | SC- | Fibroblast-CD34+ | ENSG00000120738 | ENSG00000120738.7 | 0.753816991 | 0.950413223 | 0.650764895 | 21.499587093 |
| | F1 | sublining (SC-F1) | | | | | | |
| EGR1 | SC- | Fibroblast-HLA-DRAhi | ENSG00000120738 | ENSG00000120738.7 | 0.805075452 | 0.993045897 | 0.629410522 | 21.499587093 |
| | F2 | sublining (SC-F2) | | | | | | |
| COL14A1 | SC- | Fibroblast-CD34+ | ENSG00000187955 | ENSG00000187955.7 | 0.886990583 | 0.983471074 | 0.305354267 | 21.350721991 |
| | F1 | sublining (SC-F1) | | | | | | |
| COL14A1 | SC- | Fibroblast-HLA-DRAhi | ENSG00000187955 | ENSG00000187955.7 | 0.917607177 | 0.991655076 | 0.27044158 | 21.350721991 |
| | F2 | sublining (SC-F2) | | | | | | |
| COL14A1 | SC- | Fibroblast-DKK3+ | ENSG00000187955 | ENSG00000187955.7 | 0.852312874 | 0.969298246 | 0.339203675 | 21.350721991 |
| | F3 | sublining (SC-F3) | | | | | | |
| SCARA5 | SC- | Fibroblast-CD34+ | ENSG00000168079 | ENSG00000168079.12 | 0.749160744 | 0.785123967 | 0.289653784 | 20.554709341 |
| | F1 | sublining (SC-F1) | | | | | | |
| SCARA5 | SC- | Fibroblast-DKK3+ | ENSG00000168079 | ENSG00000168079.12 | 0.819901316 | 0.890350877 | 0.309341501 | 20.554709341 |
| | F3 | sublining (SC-F3) | | | | | | |
| COL4A2 | SC- | Fibroblast-HLA-DRAhi | ENSG00000134871 | ENSG00000134871.13 | 0.841252655 | 0.866481224 | 0.187618846 | 12.249482672 |
| | F2 | sublining (SC-F2) | | | | | | |
| EGFR | SC- | Fibroblast-CD34+ | ENSG00000146648 | ENSG00000146648.11 | 0.763756222 | 0.729338843 | 0.230072464 | 10.692550335 |
| | F1 | sublining (SC-F1) | | | | | | |
| EGFR | SC- | Fibroblast-HLA-DRAhi | ENSG00000146648 | ENSG00000146648.11 | 0.803273527 | 0.820584145 | 0.191421931 | 10.692550335 |
| | F2 | sublining (SC-F2) | | | | | | |

Prime Cells

These unusual RNAs identified in blood as indicators of an RA flare, particularly those of AC3, identified a unique cell in blood samples denoted Pre-Inflammatory Mesenchymal Cells (PRIME cells). RNA sequencing of these cells confirmed that they were enriched with AC3 cluster genes, synovial fibroblast genes, and expressed classic synovial fibroblast genes such as FAP, DKK3, CDH11 as well as collagens and laminins. PRIME cells are activated just prior to flare and are then evident at flare in inflamed synovium as inflammatory sublining fibroblasts.

Cell surface markers characteristic of the PRIME cells identified and described herein are PDPN+, CD45− and CD31−. PRIME cells can be sorted or characterized as CD45−,CD31−PDPN+ cells. Also, the cell surface receptor and marker IL-17RD+ can additionally be utilized to differentiate, identify and characterize PRIME cells. IL-17RD is an AC3 gene marker (see Table 3 above). CD45 and CD31 are often present on cells in blood, therefore a blood cell which lacks both of these specific markers would be unusual. CD45 is a pan-leukocyte protein with tyrosine phosphatase activity involved in the regulation of signal transduction in hematopoiesis. CD45 is also known as protein tyrosine phosphatase, receptor type, C (PTPRC). CD45 was originally designated leukocyte common antigen, reflecting its general expression on leukocytes. CD31 represents platelet endothelial cell adhesion molecule (PECAM-1). This molecule plays a key role in removing aged neutrophils from the body, and is found on the surface of platelets, monocytes, neutrophils and some types of T cells.

In contrast, the presence of PDPN—and also of IL-17RD—on the surface of a population of cells, particularly CD45− and CD31− cells in blood, is unusual. PDPN (Podaplanin) is a well conserved mucin-type transmembrane protein and is heavily O-glycosylated with diverse distribution in human tissues. Podaplanin binds to C-type lectin receptor-2 (CLEC-2) and is associated with malignant progression and tumor metastasis in several types of cancer. Anti-podaplanin antibody has been evaluated and shown effective in LPS-induced lung injury (Lax S et al (2017) BMJ Open Respiratory Res 4:e000257.doi:10.11361/bmjresp-2017-000257). Podaplanin antibodies have been investigated in pulmonary metastasis and in malignant mesothelioma (Kato Y (2015) Oncotarget 6(34):36003-36018; Abe S et al (2013) J Immunol 190(12):6239-6249).

IL-17RD (IL-17 Receptor D), a membrane protein of the IL-17 receptor family, is a feedback loop inhibitor of fibroblast growth factor mediated Ras-MAPK signaling and ERK activation. IL-17RD binds IL-17A and mediates pro-inflammatory gene expression downstream of IL-17A.

Antibodies targeting IL-17 cytokines and their receptors are being used in treatment of some autoimmune diseases. In RA, IL-17A acts locally on synoviocytes and osteoblasts contributing to synovitis and joint disruption, Although some positive results have been seen in psoriasis and psoriatic arthritis, results with biologics targeting IL-17 in RA have been mixed, underscoring the need to identify patients or clinical/biological scenarios where therapeutics such as IL-17 biologics will be effective (Robert M and Miossec P (2019) Front Med 5:364; doi:10.3389/fmed.2018.00364; Fragoulis G E et al (2016) Ann Rev Med 67:337-353). Targeting IL-17 or IL-17D based on RNA markers and/or PRIME cell analysis may be a more effective approach to treatment of RA, particularly if administration upon recognition of an imminent flare could be implemented.

### Example 3

### Selecting Genes for Detection of PRIME Cells

In this example, a series of filtering studies were performed to focus in on more and more unique genes. We started with 625 that were increased prior to (e.g., one week or about 5-7 days prior to) flare in whole blood (Table 6), then we filtered to 283 by selecting genes that were increased in PRIME cells relative to circulating white blood cell, then we filtered again by selecting genes that were specific to synovial fibroblasts versus other synovial tissue infiltrating white blood cells.

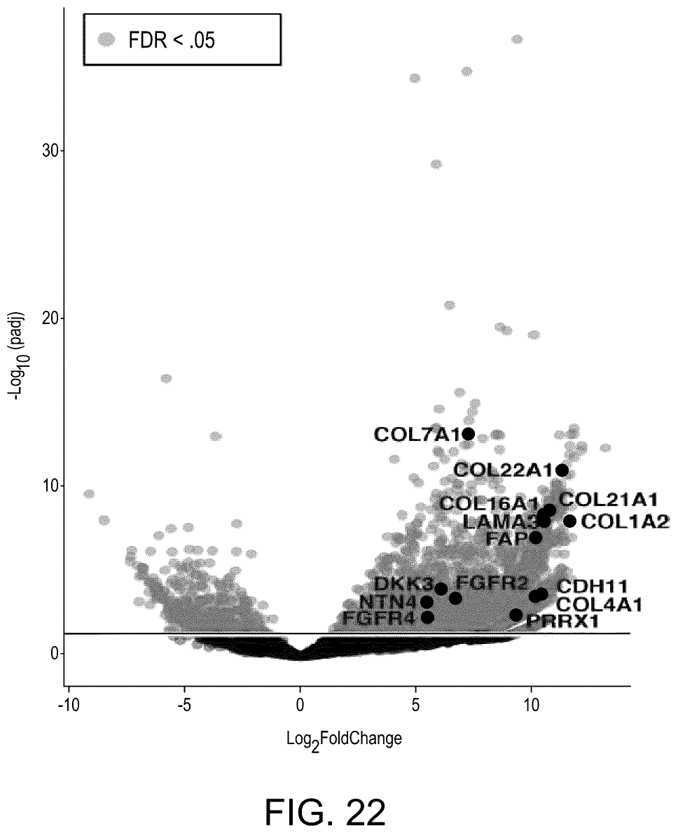

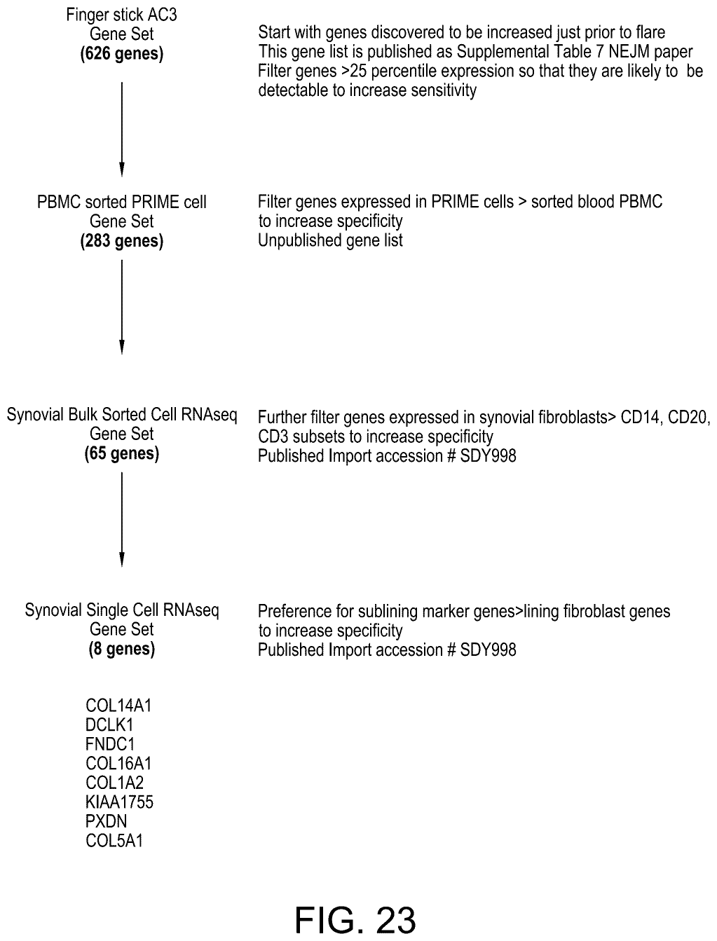

We examined a cluster of genes (AC3) that were antecedents to symptomatic flare for relevance to RA from a previously published scRNAseq dataset that were defined into 18 synovial cell types. See, Zhang, F., et al., Defining inflammatory cell states in rheumatoid arthritis joint synovial tissues by integrating single-cell transcriptomics and mass cytometry. Nat Immunol, 2019. 20(7): p. 928-942. We identified 625 transcripts were increased just prior to flare and 194 of these were also expressed by synovial sublining fibroblasts. See Table 6. We also examined the blood of 23 RA patients for the presence of an outstanding single synovial signature of these cells (CD45−/CD31−/PDPN+) and found these cells are enriched in the blood of RA patients (FIG. 4D). Comparing sorted RNAseq signatures of these PDPN+ cells with AC3 gene clusters and found these PDPN+ cells have enriched expression of synovial fibroblast genes such as FAP, DKK3, CDH11, as well as collagens and laminins. FIG. 22). Discovering that AC3 genes are also enriched in synovial fibroblasts was useful because it increased the specificity of the signature since fibroblasts do not normally circulate in blood.

A process illustrated in FIG. 24 is used to refine the dataset to narrow down the number of markers we used. We began with the set of 625 AC3 genes that were previously found to have decreased expression during RA flares, and refined this to the smaller set of the most highly expressed genes (top quartile) that would be easiest to detect by PCR or FACS. We then filtered those genes that were enriched in PRIME cells relative to other hematopoietic cells, using the differential volcano plotting strategy (PDPN+ vs CD45+) as in FIG. 22 (focusing on the equivalent group of genes to the right of the plot, with significant fold changes (>0) relative to hematopoietic cells). This analysis yielded an (unpublished) dataset of 283 genes enriched with PRIME cell markers. See Table 10, which shows expression of a panel of 283 genes that were significantly increased in whole blood prior to flare, and these 283 genes were also significantly increased in PRIME cells compared to peripheral blood mononuclear cells in the same individual, as indicated by that the significance of Log 2FC PRIME cells/PBMC (“padj_prime”) less than 0.05.

We next further refined this dataset by selecting the subgroup of 283 genes identified that are also published synovial marker genes (CD14, CD20, CD3) in published datasets (accession #SDY998). This analysis yielded a dataset of 65 genes. See Table 11, which shows a panel of 65 genes i) significantly increased in expression levels in whole blood prior to flare, ii) significantly increased in expression levels in PRIME cells compared to peripheral blood mononuclear cells, and iii) significantly increased in expression levels in synovial tissue fibroblasts compared to synovial tissue monocytes, B cells and T cells, as indicated by that the ratio of gene expression in fibroblasts to the sum of gene expression in B cells, Monocytes and T cells (“fibroblast/(B+M+T)” is greater than 1).

Finally, we further refined this dataset by selecting the subgroup of 65 genes that overlapped published single-cell RNAseq data (accession #SDY998) that were enriched for synovial sublining genes relative to fibroblast genes. See, Zhang, F., et al., Nat Immunol, 2019. 20(7): p. 928-942. We suspect synovial sublining genes to be most relevant to PRIME cells and RA pathophysiology. This analysis yielded a dataset of 8 genes, which are shown in Table 12, which shows a panel of 8 genes that were i) significantly increased in expression levels in whole blood prior to flare, ii) significantly increased in expression levels in PRIME cells compared to peripheral blood mononuclear cells, iii) significantly increased in expression levels in synovial tissue fibroblasts compared to synovial tissue monocytes, B cells and T cells, iv) expressed in sublining fibroblasts in significantly higher level than in lining layer synovial fibroblasts.

The AC3 markers and the primers that can be used to detect these RNA expression in RNAseq are shown in Table 13.

TABLE 10
283 genes in PRIME cells

| | | Log2FoldChage | Significance of Log2FC |
|---|---|---|---|
| | GENE | PRIME | PRIME cells/PBMC |
| Ensemble GENE ID | SYMBOL | cells/PBMC | (“padj_prime”) |
| ENSG00000181143.11 | MUC16 | 10.0855436 | 7.9386E−20 |
| ENSG00000171435.9 | KSR2 | 11.8018796 | 3.0049E−14 |
| ENSG00000165323.11 | FAT3 | 12.1409422 | 3.3332E−13 |
| ENSG00000138759.13 | FRAS1 | 11.4663395 | 4.2845E−12 |
| ENSG00000169436.12 | COL22A1 | 11.2957133 | 8.8587E−12 |
| ENSG00000119547.5 | ONECUT2 | 11.6439194 | 1.6669E−11 |
| ENSG00000188162.6 | OTOG | 11.4779014 | 1.9696E−11 |
| ENSG00000133454.11 | MYO18B | 8.64409108 | 1.3105E−10 |
| ENSG00000205592.8 | MUC19 | 10.6062292 | 2.0866E−10 |
| ENSG00000142449.8 | FBN3 | 11.2240728 | 2.2122E−10 |
| ENSG00000078295.11 | ADCY2 | 11.2421588 | 3.32E−10 |
| ENSG00000130226.12 | DPP6 | 11.2046162 | 3.32E−10 |
| ENSG00000157214.9 | STEAP2 | 11.068942 | 3.4359E−10 |
| ENSG00000137766.12 | UNC13C | 10.7491846 | 5.9062E−10 |
| ENSG00000261115.1 | TMEM178B | 7.88414812 | 6.2418E−10 |
| ENSG00000170927.10 | PKHD1 | 10.944145 | 7.3843E−10 |
| ENSG00000178568.9 | ERBB4 | 10.7872402 | 1.3478E−09 |
| ENSG00000128573.18 | FOXP2 | 10.8375719 | 1.3769E−09 |
| ENSG00000002746.9 | HECW1 | 10.7347811 | 1.4252E−09 |
| ENSG00000124749.12 | COL21A1 | 10.7884891 | 2.1212E−09 |
| ENSG00000069431.6 | ABCC9 | 11.307877 | 2.5167E−09 |
| ENSG00000120251.14 | GRIA2 | 10.6331048 | 3.2019E−09 |
| ENSG00000158258.11 | CLSTN2 | 11.1567218 | 3.6938E−09 |
| ENSG00000144908.9 | ALDH1L1 | 9.8829159 | 3.7495E−09 |
| ENSG00000084636.12 | COL16A1 | 10.5120707 | 3.7567E−09 |
| ENSG00000088340.11 | FER1L4 | 10.5353056 | 4.4615E−09 |
| ENSG00000164692.13 | COL1A2 | 11.6422745 | 9.7979E−09 |
| ENSG00000174963.13 | ZIC4 | 10.574388 | 2.5507E−08 |
| ENSG00000091656.11 | ZFHX4 | 9.57608804 | 3.272E−08 |
| ENSG00000079841.14 | RIMS1 | 10.2678036 | 3.3081E−08 |
| ENSG00000039139.9 | DNAH5 | 11.1877289 | 3.4819E−08 |
| ENSG00000170777.9 | TPD52L3 | 10.4382661 | 4.0608E−08 |
| ENSG00000198788.7 | MUC2 | 10.5539222 | 5.5502E−08 |
| ENSG00000144648.9 | ACKR2 | 10.1770267 | 6.2846E−08 |
| ENSG00000148357.12 | HMCN2 | 11.2528227 | 6.4777E−08 |
| ENSG00000123243.10 | ITIH5 | 10.356014 | 7.5441E−08 |
| ENSG00000187527.6 | ATP13A5 | 10.1275523 | 7.9099E−08 |
| ENSG00000163395.12 | IGFN1 | 10.6666204 | 1.0443E−07 |
| ENSG00000130508.6 | PXDN | 7.7050129 | 1.0612E−07 |
| ENSG00000187068.2 | C3orf70 | 9.67322943 | 1.1207E−07 |
| ENSG00000130477.10 | UNC13A | 11.348469 | 1.5587E−07 |
| ENSG00000236445.2 | LINC00608 | 10.0271293 | 1.8548E−07 |
| ENSG00000165970.7 | SLC6A5 | 10.208198 | 3.5377E−07 |
| ENSG00000186862.12 | PDZD7 | 6.74925767 | 4.2539E−07 |
| ENSG00000130635.11 | COL5A1 | 6.30822442 | 4.7142E−07 |
| ENSG00000172752.10 | COL6A5 | 10.5737724 | 5.2374E−07 |
| ENSG00000009709.7 | PAX7 | 10.1290357 | 7.2306E−07 |
| ENSG00000152822.9 | GRM1 | 10.3735166 | 7.6561E−07 |
| ENSG00000119283.11 | TRIM67 | 9.85988714 | 9.3759E−07 |
| ENSG00000095637.16 | SORBS1 | 7.4923642 | 9.6242E−07 |
| ENSG00000132297.7 | HHLA1 | 10.4722764 | 1.0254E−06 |
| ENSG00000185038.9 | MROH2A | 10.2250561 | 1.0374E−06 |
| ENSG00000157423.13 | HYDIN | 7.24026448 | 1.0532E−06 |
| ENSG00000214929.3 | SPATA31D1 | 9.78447505 | 1.24E−06 |
| ENSG00000162631.14 | NTNG1 | 8.04063292 | 1.2555E−06 |
| ENSG00000082175.10 | PGR | 10.271583 | 1.8283E−06 |
| ENSG00000227036.2 | LINC00511 | 7.28217824 | 2.1972E−06 |
| ENSG00000170426.1 | SDR9C7 | 10.1702866 | 2.2495E−06 |
| ENSG00000166473.12 | PKD1L2 | 9.79452466 | 2.6027E−06 |
| ENSG00000106078.13 | COBL | 8.88603918 | 2.7912E−06 |
| ENSG00000177511.5 | ST8SIA3 | 10.3401945 | 3.36E−06 |
| ENSG00000177551.5 | NHLH2 | 10.2528811 | 3.9526E−06 |
| ENSG00000185739.9 | SRL | 9.6130637 | 5.3822E−06 |
| ENSG00000234512.1 | TLR12P | 9.8139391 | 6.1124E−06 |
| ENSG00000236824.1 | BCYRN1 | 9.15544222 | 6.7792E−06 |
| ENSG00000135454.9 | B4GALNT1 | 9.59499813 | 1.0344E−05 |
| ENSG00000018625.10 | ATP1A2 | 9.67355875 | 1.0733E−05 |
| ENSG00000100033.11 | PRODH | 10.101159 | 1.1679E−05 |
| ENSG00000170442.7 | KRT86 | 7.93055895 | 1.1925E−05 |
| ENSG00000157927.12 | RADIL | 9.08000698 | 1.2249E−05 |
| ENSG00000183580.8 | FBXL7 | 11.1382412 | 1.4708E−05 |
| ENSG00000137648.12 | TMPRSS4 | 9.00886605 | 1.4852E−05 |
| ENSG00000136535.10 | TBR1 | 9.97859539 | 1.6677E−05 |
| ENSG00000105357.11 | MYH14 | 9.51054359 | 2.0034E−05 |
| ENSG00000159337.6 | PLA2G4D | 11.7111821 | 2.9149E−05 |
| ENSG00000163995.14 | ABLIM2 | 5.37676509 | 3.0548E−05 |
| ENSG00000125740.9 | FOSB | −3.74259281 | 3.0548E−05 |
| ENSG00000148942.10 | SLC5A12 | 9.57605369 | 3.1642E−05 |
| ENSG00000174498.9 | IGDCC3 | 9.72070592 | 3.3715E−05 |
| ENSG00000237515.6 | SHISA9 | 10.7837773 | 3.3994E−05 |
| ENSG00000171487.10 | NLRP5 | 9.48549744 | 3.5037E−05 |
| ENSG00000101004.10 | NINL | 5.93645294 | 3.5037E−05 |
| ENSG00000168079.12 | SCARA5 | 7.50628313 | 3.5922E−05 |
| ENSG00000060709.9 | RIMBP2 | 7.44688832 | 3.6624E−05 |
| ENSG00000171587.10 | DSCAM | 9.29784851 | 4.7157E−05 |
| ENSG00000175267.10 | VWA3A | 6.86579745 | 5.3792E−05 |
| ENSG00000175267.10 | VWA3A | 6.86579745 | 5.3792E−05 |
| ENSG00000175267.10 | VWA3A | 6.86579745 | 5.3792E−05 |
| ENSG00000175267.10 | VWA3A | 6.86579745 | 5.3792E−05 |
| ENSG00000175267.10 | VWA3A | 6.86579745 | 5.3792E−05 |
| ENSG00000175267.10 | VWA3A | 6.86579745 | 5.3792E−05 |
| ENSG00000175267.10 | VWA3A | 6.86579745 | 5.3792E−05 |
| ENSG00000175267.10 | VWA3A | 6.86579745 | 5.3792E−05 |
| ENSG00000175267.10 | VWA3A | 6.86579745 | 5.3792E−05 |
| ENSG00000175267.10 | VWA3A | 6.86579745 | 5.3792E−05 |
| ENSG00000175267.10 | VWA3A | 6.86579745 | 5.3792E−05 |
| ENSG00000175267.10 | VWA3A | 6.86579745 | 5.3792E−05 |
| ENSG00000175267.10 | VWA3A | 6.86579745 | 5.3792E−05 |
| ENSG00000175267.10 | VWA3A | 6.86579745 | 5.3792E−05 |
| ENSG00000175267.10 | VWA3A | 6.86579745 | 5.3792E−05 |
| ENSG00000175267.10 | VWA3A | 6.86579745 | 5.3792E−05 |
| ENSG00000015520.10 | NPC1L1 | 9.88092763 | 6.0092E−05 |
| ENSG00000164904.11 | ALDH7A1 | 5.47673693 | 6.1387E−05 |
| ENSG00000183876.8 | ARSI | 9.31161098 | 6.5162E−05 |
| ENSG00000138162.13 | TACC2 | 10.9079109 | 6.8589E−05 |
| ENSG00000060656.15 | PTPRU | 7.27711274 | 6.8677E−05 |
| ENSG00000161243.4 | FBXO27 | 7.24482957 | 7.9276E−05 |
| ENSG00000169169.10 | CPT1C | 8.09351669 | 7.9973E−05 |
| ENSG00000257008.2 | GPR142 | 8.56229961 | 8.5521E−05 |
| ENSG00000101680.9 | LAMA1 | 10.6992197 | 0.00010128 |
| ENSG00000178171.6 | AMER3 | 10.6794817 | 0.00010499 |
| ENSG00000111799.16 | COL12A1 | 10.7382323 | 0.00010546 |
| ENSG00000081248.6 | CACNA1S | 9.80041569 | 0.00012856 |
| ENSG00000164093.11 | PITX2 | 10.422723 | 0.00012858 |
| ENSG00000187094.7 | CCK | 9.16123651 | 0.0001322 |
| ENSG00000116833.9 | NR5A2 | 10.5500444 | 0.00016126 |
| ENSG00000174502.14 | SLC26A9 | 10.5281245 | 0.00016126 |
| ENSG00000165300.6 | SLITRK5 | 7.99902598 | 0.00018194 |
| ENSG00000112499.7 | SLC22A2 | 10.3025109 | 0.00019563 |
| ENSG00000170381.8 | SEMA3E | 10.4226159 | 0.00021084 |
| ENSG00000146005.3 | PSD2 | 6.72463576 | 0.00022891 |
| ENSG00000169126.11 | ARMC4 | 10.4923335 | 0.00022891 |
| ENSG00000229147.1 | SMPD4P2 | 10.4382553 | 0.00026303 |
| ENSG00000134871.13 | COL4A2 | 7.74572011 | 0.00028096 |
| ENSG00000122778.5 | KIAA1549 | 9.14112261 | 0.00034123 |
| ENSG00000146839.14 | ZAN | 9.1693989 | 0.0003457 |
| ENSG00000095587.8 | TLL2 | 9.98109668 | 0.00035474 |
| ENSG00000158125.5 | XDH | 10.2397661 | 0.00037082 |
| ENSG00000250420.4 | AACSP1 | 8.93443911 | 0.00037467 |
| ENSG00000139144.5 | PIK3C2G | 9.19770133 | 0.00037518 |
| ENSG00000172350.5 | ABCG4 | 10.2578105 | 0.00038747 |
| ENSG00000150893.9 | FREM2 | 10.1857757 | 0.00041483 |
| ENSG00000179766.13 | ATP8B5P | 10.2928738 | 0.00041559 |
| ENSG00000148408.8 | CACNA1B | 9.12553182 | 0.00042841 |
| ENSG00000133083.10 | DCLK1 | 10.4763853 | 0.0004357 |
| ENSG00000148702.10 | HABP2 | 9.93507316 | 0.00043749 |
| ENSG00000146648.11 | EGFR | 9.32416759 | 0.00047166 |
| ENSG00000145832.8 | SLC25A48 | 10.3732943 | 0.00048682 |
| ENSG00000171533.7 | MAP6 | 10.1100079 | 0.00050118 |
| ENSG00000245248.3 | USP2-AS1 | 10.3915304 | 0.00050576 |
| ENSG00000166444.13 | ST5 | 7.03013101 | 0.00051906 |
| ENSG00000183856.6 | IQGAP3 | 7.66144499 | 0.00055556 |
| ENSG00000167654.13 | ATCAY | 9.02455288 | 0.00056242 |
| ENSG00000168356.7 | SCN11A | 7.9058759 | 0.00059864 |
| ENSG00000186487.13 | MYT1L | 10.3013562 | 0.00060125 |
| ENSG00000139767.4 | SRRM4 | 9.1322541 | 0.00060437 |
| ENSG00000147689.12 | FAM83A | 8.68477341 | 0.00064925 |
| ENSG00000187955.7 | COL14A1 | 10.3457395 | 0.00065046 |
| ENSG00000204661.5 | C5orf60 | 7.98854519 | 0.00065089 |
| ENSG00000111262.4 | KCNA1 | 10.0799494 | 0.00065233 |
| ENSG00000123572.12 | NRK | 9.77398063 | 0.00068182 |
| ENSG00000242866.5 | STRC | 7.89404297 | 0.00069439 |
| ENSG00000108018.11 | SORCS1 | 10.4860855 | 0.00074009 |
| ENSG00000237125.4 | HAND2-AS1 | 9.93585238 | 0.00080912 |
| ENSG00000143355.11 | LHX9 | 9.07176393 | 0.00080992 |
| ENSG00000197893.9 | NRAP | 10.1582696 | 0.00087519 |
| ENSG00000110786.13 | PTPN5 | 10.1020442 | 0.00087704 |
| ENSG00000177103.9 | DSCAML1 | 6.69871332 | 0.00097226 |
| ENSG00000101203.12 | COL20A1 | 10.0926992 | 0.00103622 |
| ENSG00000152910.14 | CNTNAP4 | 9.58161879 | 0.00111905 |
| ENSG00000169344.11 | UMOD | 9.57480249 | 0.00119192 |
| ENSG00000187123.10 | LYPD6 | 9.6224591 | 0.00121851 |
| ENSG00000196091.8 | MYBPC1 | 9.64996378 | 0.00123954 |
| ENSG00000006788.7 | MYH13 | 9.77503542 | 0.00132102 |
| ENSG00000115648.9 | MLPH | 9.94753399 | 0.00135935 |
| ENSG00000156222.7 | SLC28A1 | 9.47454797 | 0.00149201 |
| ENSG00000151224.8 | MAT1A | 9.67253653 | 0.00155075 |
| ENSG00000188488.9 | SERPINA5 | 9.74848797 | 0.00155075 |
| ENSG00000162738.5 | VANGL2 | 8.18945891 | 0.00156613 |
| ENSG00000172995.12 | ARPP21 | 9.46656103 | 0.00176545 |
| ENSG00000169876.9 | MUC17 | 9.63391559 | 0.00181592 |
| ENSG00000198010.7 | DLGAP2 | 9.73919587 | 0.00181592 |
| ENSG00000165973.13 | NELL1 | 9.73919587 | 0.00181592 |
| ENSG00000198597.4 | ZNF536 | 9.63391559 | 0.00181592 |
| ENSG00000130287.8 | NCAN | 9.51783924 | 0.00188441 |
| ENSG00000143107.4 | FNDC7 | 6.94089227 | 0.00226789 |
| ENSG00000089199.5 | CHGB | 8.37600881 | 0.00226861 |
| ENSG00000138650.6 | PCDH10 | 9.52030811 | 0.0022794 |
| ENSG00000004948.9 | CALCR | 9.56275025 | 0.0022794 |
| ENSG00000091128.8 | LAMB4 | 9.56275025 | 0.0022794 |
| ENSG00000169862.14 | CTNND2 | 9.59457564 | 0.00239769 |
| ENSG00000156687.6 | UNC5D | 9.83690372 | 0.00239769 |
| ENSG00000179008.4 | C14orf39 | 9.59457564 | 0.00239769 |
| ENSG00000268388.1 | FENDRR | 9.59457564 | 0.00239769 |
| ENSG00000205922.4 | ONECUT3 | 9.59457564 | 0.00239769 |
| ENSG00000142408.2 | CACNG8 | 9.45372393 | 0.00243109 |
| ENSG00000151474.15 | FRMD4A | 4.26915376 | 0.00252682 |
| ENSG00000144730.12 | IL-17RD | 9.47740902 | 0.00256297 |
| ENSG00000178031.11 | ADAMTSL1 | 9.30109081 | 0.00256297 |
| ENSG00000197085.7 | NPSR1-AS1 | 9.82478961 | 0.00273892 |
| ENSG00000165566.11 | AMER2 | 9.82478961 | 0.00273892 |
| ENSG00000174279.4 | EVX2 | 9.23221912 | 0.00287805 |
| ENSG00000160460.11 | SPTBN4 | 7.90179181 | 0.00297008 |
| ENSG00000139865.12 | TTC6 | 9.43374983 | 0.00335063 |
| ENSG00000054356.9 | PTPRN | 9.35015286 | 0.00361961 |
| ENSG00000112186.7 | CAP2 | 9.35015286 | 0.00361961 |
| ENSG00000259156.3 | CHEK2P2 | 9.35015286 | 0.00361961 |
| ENSG00000185823.2 | NPAP1 | 9.35015286 | 0.00361961 |
| ENSG00000106304.11 | SPAM1 | 9.2402676 | 0.00370324 |
| ENSG00000159251.6 | ACTC1 | 9.64814054 | 0.00376653 |
| ENSG00000224743.2 | TEX26-AS1 | 9.46689834 | 0.00384057 |
| ENSG00000183317.12 | EPHA10 | 7.75193464 | 0.00387013 |
| ENSG00000166391.10 | MOGAT2 | 9.30177815 | 0.00387013 |
| ENSG00000188803.9 | SHISA6 | 9.30177815 | 0.00387013 |
| ENSG00000135931.13 | ARMC9 | 7.57025316 | 0.00388201 |
| ENSG00000119125.12 | GDA | 9.39828095 | 0.00388201 |
| ENSG00000006210.6 | CX3CL1 | 9.39828095 | 0.00388201 |
| ENSG00000145242.9 | EPHA5 | 9.55424849 | 0.00396734 |
| ENSG00000178645.8 | C10orf53 | 9.42301327 | 0.00406126 |
| ENSG00000124092.7 | CTCFL | 9.09328581 | 0.00422446 |
| ENSG00000114019.10 | AMOTL2 | 8.07387917 | 0.00452094 |
| ENSG00000101871.10 | MID1 | 8.04439073 | 0.00461599 |
| ENSG00000115041.8 | KCNIP3 | 9.25262887 | 0.0047579 |
| ENSG00000188886.3 | ASTL | 4.75628627 | 0.0047579 |
| ENSG00000070886.6 | EPHA8 | 9.10352401 | 0.00487397 |
| ENSG00000142611.12 | PRDM16 | 6.26811025 | 0.00497995 |
| ENSG00000164694.12 | FNDC1 | 9.10174997 | 0.00614899 |
| ENSG00000185737.8 | NRG3 | 9.10174997 | 0.00614899 |
| ENSG00000196136.11 | SERPINA3 | 9.15752037 | 0.00614899 |
| ENSG00000171804.5 | WDR87 | 9.15752037 | 0.00614899 |
| ENSG00000124440.11 | HIF3A | 9.10174997 | 0.00614899 |
| ENSG00000006283.13 | CACNA1G | 7.29179036 | 0.00676935 |
| ENSG00000105290.7 | APLP1 | 7.01702864 | 0.00703809 |
| ENSG00000237636.2 | ANKRD26P3 | 9.04536192 | 0.00728432 |
| ENSG00000182256.8 | GABRG3 | 9.04536192 | 0.00728432 |
| ENSG00000140279.8 | DUOX2 | 9.04536192 | 0.00728432 |
| ENSG00000137573.9 | SULF1 | 9.20219199 | 0.00738189 |
| ENSG00000224059.1 | HSPA8P16 | 10.5118456 | 0.00760264 |
| ENSG00000156103.11 | MMP16 | 9.21388586 | 0.00776909 |
| ENSG00000091137.7 | SLC26A4 | 7.81519145 | 0.00821577 |
| ENSG00000143469.12 | SYT14 | 9.03182628 | 0.00838462 |
| ENSG00000183668.13 | PSG9 | 10.4307525 | 0.00992099 |
| ENSG00000120332.11 | TNN | 8.86959591 | 0.01092105 |
| ENSG00000230873.4 | STMND1 | 8.98752678 | 0.01092105 |
| ENSG00000106648.9 | GALNTL5 | 8.98752678 | 0.01092105 |
| ENSG00000141668.5 | CBLN2 | 8.98752678 | 0.01092105 |
| ENSG00000104967.6 | NOVA2 | 8.98752678 | 0.01092105 |
| ENSG00000174358.11 | SLC6A19 | 8.87308109 | 0.01142733 |
| ENSG00000185313.6 | SCN10A | 8.80310292 | 0.01200226 |
| ENSG00000125492.5 | BARHL1 | 8.80310292 | 0.01200226 |
| ENSG00000140527.10 | WDR93 | 8.80310292 | 0.01200226 |
| ENSG00000179270.6 | C2orf71 | 8.93637024 | 0.01274995 |
| ENSG00000165379.9 | LRFN5 | 8.93637024 | 0.01274995 |
| ENSG00000161270.15 | NPHS1 | 8.71784874 | 0.01285489 |
| ENSG00000226057.2 | PHF2P2 | 5.94495217 | 0.01427779 |
| ENSG00000161940.6 | BCL6B | 8.58900305 | 0.0148154 |
| ENSG00000100065.10 | CARD10 | 7.61171168 | 0.0148154 |
| ENSG00000116721.9 | PRAMEF1 | 8.73511268 | 0.01716161 |
| ENSG00000183242.7 | WT1-AS | 8.73511268 | 0.01716161 |
| ENSG00000104055.10 | TGM5 | 8.73511268 | 0.01716161 |
| ENSG00000149633.7 | KIAA1755 | 8.73511268 | 0.01716161 |
| ENSG00000250423.2 | KIAA1210 | 8.73511268 | 0.01716161 |
| ENSG00000077522.8 | ACTN2 | 8.51176056 | 0.02199638 |
| ENSG00000226068.1 | HNRNPA3P4 | 8.59391365 | 0.02260172 |
| ENSG00000167723.10 | TRPV3 | 4.55901439 | 0.02266943 |
| ENSG00000223414.2 | LINC00473 | 9.76187181 | 0.02336738 |
| ENSG00000197406.6 | DIO3 | 9.80247266 | 0.02410841 |
| ENSG00000132321.12 | IQCA1 | 5.74183061 | 0.02461755 |
| ENSG00000138892.7 | TTLL8 | 9.669433 | 0.02680035 |
| ENSG00000185974.6 | GRK1 | 8.42879991 | 0.02836865 |
| ENSG00000197079.4 | KRT35 | 8.42879991 | 0.02836865 |
| ENSG00000106689.6 | LHX2 | 9.5706152 | 0.03023653 |
| ENSG00000183908.5 | LRRC55 | 9.5706152 | 0.03023653 |
| ENSG00000168907.9 | PLA2G4F | 9.5706152 | 0.03023653 |
| ENSG00000144115.12 | THNSL2 | 4.66118466 | 0.03458112 |
| ENSG00000138675.12 | FGF5 | 9.51283135 | 0.03534253 |
| ENSG00000132975.6 | GPR12 | 9.50675823 | 0.03706847 |
| ENSG00000257907.2 | EEF1A1P17 | 5.43581118 | 0.03999134 |
| ENSG00000134240.7 | HMGCS2 | 9.34987098 | 0.04100703 |
| ENSG00000185303.11 | SFTPA2 | 9.34987098 | 0.04100703 |
| ENSG00000115155.12 | OTOF | 4.23436155 | 0.04404224 |
| ENSG00000184908.13 | CLCNKB | 9.34282178 | 0.04416808 |
| ENSG00000260305.1 | NTRK3-AS1 | 9.34282178 | 0.04416808 |
| ENSG00000141434.7 | MEP1B | 9.34282178 | 0.04416808 |
| ENSG00000116748.15 | AMPD1 | 8.14697124 | 0.04465983 |
| ENSG00000166159.6 | LRTM2 | 8.14697124 | 0.04465983 |
| ENSG00000139220.12 | PPFIA2 | 8.14697124 | 0.04465983 |
| ENSG00000089116.3 | LHX5 | 8.14697124 | 0.04465983 |
| ENSG00000109101.3 | FOXN1 | 8.14697124 | 0.04465983 |
| ENSG00000183287.9 | CCBE1 | 8.14697124 | 0.04465983 |
| ENSG00000196361.5 | ELAVL3 | 8.07040565 | 0.04534929 |
| ENSG00000154099.13 | DNAAF1 | 6.27100113 | 0.04646486 |
| ENSG00000132972.13 | RNF17 | 6.23370242 | 0.04856254 |

TABLE 11
Comparison of gene expression level of 65 genes in sorted synovial cells

| | Mean | Mean | Mean | Mean | |
|---|---|---|---|---|---|
| Analysis | expression | expression in | expression in | expression | fibroblast/ |
| Columns | in B cell | Fibroblast | Monocyte | in T cell | (B + M + T) |
| DIO3 | 0.005898 | 0.568446 | 0.012513 | 0.012674 | 18.28676 |
| ZIC4 | 0.040445 | 2.794642 | 0.082367 | 0.048743 | 16.29007 |
| KCNA1 | 0.007163 | 0.845192 | 0.028432 | 0.020589 | 15.04338 |
| FBXL7 | 0.01174 | 1.390057 | 0.041241 | 0.041037 | 14.7851 |
| FNDC1 | 0.04824 | 3.390032 | 0.113013 | 0.072297 | 14.5152 |
| ARSI | 0.010528 | 0.896302 | 0.041211 | 0.0208 | 12.35612 |
| NRAP | 0.002079 | 0.084748 | 0.004413 | 0.00293 | 8.994018 |
| PTPRU | 0.017946 | 1.587554 | 0.110434 | 0.07467 | 7.818552 |
| C14orf39 | 0.020927 | 1.169926 | 0.078181 | 0.050739 | 7.807468 |
| PGR | 0.021031 | 0.908659 | 0.031519 | 0.070138 | 7.406261 |
| COL12A1 | 0.101297 | 4.658146 | 0.295869 | 0.23946 | 7.316921 |
| SEMA3E | 0.116105 | 4.591674 | 0.317588 | 0.223195 | 6.990037 |
| EGFR | 0.046741 | 3.586856 | 0.302146 | 0.202491 | 6.505261 |
| STEAP2 | 0.159893 | 4.446441 | 0.282877 | 0.327752 | 5.770686 |
| NRK | 0.016676 | 0.440618 | 0.023194 | 0.037504 | 5.694656 |
| SERPINA5 | 0.073307 | 2.938701 | 0.189757 | 0.27953 | 5.416022 |
| KCNIP3 | 0.06369 | 1.527446 | 0.190652 | 0.053125 | 4.967833 |
| NPC1L1 | 0.012326 | 0.231116 | 0.013956 | 0.025922 | 4.427196 |
| LINC00473 | 0.084001 | 1.670193 | 0.123524 | 0.177062 | 4.342824 |
| COL14A1 | 0.377592 | 6.658365 | 0.659155 | 0.61882 | 4.021805 |
| COL5A1 | 0.195882 | 5.152502 | 0.406335 | 0.76069 | 3.780524 |
| AMOTL2 | 0.212456 | 4.011784 | 0.342912 | 0.514269 | 3.750603 |
| CAP2 | 0.018429 | 1.046753 | 0.070727 | 0.197247 | 3.654824 |
| COL6A5 | 0.031141 | 0.433145 | 0.039923 | 0.049849 | 3.58229 |
| KIAA1755 | 0.210203 | 3.295953 | 0.386915 | 0.340558 | 3.515026 |
| SCARA5 | 0.397929 | 7.948255 | 1.207422 | 0.828519 | 3.265686 |
| IQCA1 | 0.121789 | 1.492112 | 0.153691 | 0.202833 | 3.119531 |
| PSD2 | 0.023408 | 0.281469 | 0.040438 | 0.028933 | 3.033749 |
| LHX9 | 0.051171 | 1.053767 | 0.148808 | 0.152183 | 2.992278 |
| SERPINA3 | 0.20254 | 3.073661 | 0.409223 | 0.451665 | 2.890334 |
| IL-17RD | 0.051696 | 0.85624 | 0.096036 | 0.14902 | 2.885379 |
| ADAMTSL1 | 0.420496 | 4.552426 | 0.558634 | 0.604709 | 2.874299 |
| MID1 | 0.306231 | 2.183956 | 0.267049 | 0.208137 | 2.794864 |
| DNAH5 | 0.039732 | 0.46464 | 0.05278 | 0.079678 | 2.698414 |
| CCBE1 | 0.178558 | 1.739894 | 0.189602 | 0.283328 | 2.670645 |
| RADIL | 0.050587 | 1.022948 | 0.185301 | 0.172746 | 2.503331 |
| CX3CL1 | 0.140927 | 1.802906 | 0.24785 | 0.335124 | 2.49054 |
| ITIH5 | 0.353934 | 3.49945 | 0.471817 | 0.617776 | 2.424235 |
| MROH2A | 0.004796 | 0.041373 | 0.009127 | 0.004261 | 2.275224 |
| LRFN5 | 0.175952 | 1.073433 | 0.194453 | 0.103999 | 2.262694 |
| COL16A1 | 0.54871 | 5.199992 | 0.794715 | 1.037929 | 2.183629 |
| HECW1 | 0.269484 | 1.282021 | 0.15447 | 0.174378 | 2.14266 |
| ACTCI | 0.038088 | 0.537753 | 0.104089 | 0.111694 | 2.118223 |
| COL21A1 | 0.275376 | 1.631425 | 0.323602 | 0.191552 | 2.063711 |
| FAT3 | 0.035617 | 0.291937 | 0.058087 | 0.049205 | 2.042804 |
| RIMBP2 | 0.078437 | 0.580567 | 0.118093 | 0.088082 | 2.039855 |
| SULF1 | 0.828528 | 8.126898 | 1.719874 | 1.537547 | 1.988987 |
| COL22A1 | 0.227464 | 3.068647 | 0.76669 | 0.562446 | 1.971379 |
| COBL | 0.067772 | 0.779173 | 0.122777 | 0.2072 | 1.958957 |
| DCLK1 | 0.386336 | 2.825789 | 0.532844 | 0.570433 | 1.896995 |
| SORBS1 | 0.23154 | 1.78414 | 0.302128 | 0.414584 | 1.881503 |
| NALCN | 0.166223 | 1.330218 | 0.201117 | 0.36335 | 1.820496 |
| B4GALNT1 | 0.178822 | 1.051208 | 0.207279 | 0.206557 | 1.773718 |
| PXDN | 0.651408 | 3.949243 | 0.757244 | 0.998328 | 1.640745 |
| COL1A2 | 1.634163 | 11.30866 | 3.133569 | 2.358078 | 1.587 |
| TACC2 | 0.574502 | 3.967579 | 0.874585 | 1.054203 | 1.584946 |
| TNN | 0.023226 | 0.236661 | 0.033165 | 0.093681 | 1.57698 |
| ACKR2 | 0.441855 | 2.320834 | 0.563379 | 0.480746 | 1.56182 |
| NTNG1 | 1.350033 | 2.578216 | 0.154202 | 0.193997 | 1.518177 |
| FGF5 | 0.226262 | 0.688995 | 0.129544 | 0.103465 | 1.500193 |
| PPFIA2 | 0.463127 | 1.777311 | 0.447252 | 0.275962 | 1.498146 |
| C2orf71 | 0.006667 | 0.062577 | 0.013228 | 0.023256 | 1.450165 |
| DUOX2 | 0.095781 | 0.662764 | 0.133862 | 0.233645 | 1.430565 |
| HMCN2 | 0.259065 | 1.440545 | 0.304932 | 0.459232 | 1.407843 |
| CLCNKB | 0.031294 | 0.107496 | 0.013261 | 0.031801 | 1.407827 |

Note:
“fibroblast/(B + M + T)” refers to the ratio of gene expression in fibroblasts to the sum of gene expression in B cells, Monocytes and T cells.

TABLE 12
8 marker genes and their expression
in 4 subsets of synovial fibroblasts

| Gene Name | SC-F1 | SC-F2 | SC-F3 | SC-F4 |
|---|---|---|---|---|
| COL14A1 | 1 | 0 | 0 | 0 |
| DCLK1 | 1 | 0 | 0 | 0 |
| FNDC1 | 1 | 0 | 0 | 0 |
| COL16A1 | 0 | 1 | 0 | 0 |
| COL1A2 | 0 | 1 | 0 | 0 |
| KIAA1755 | 0 | 1 | 0 | 0 |
| PXDN | 0 | 1 | 0 | 0 |
| COL5A1 | 0 | 0 | 1 | 0 |

Note:
SC-F1 refers to fibroblast-CD34+ sublining cells; SC-F2 refers to fibroblast-HLA-DRAhi sublining cells; SC-F3 refers to fibroblast-DKK3+ sublining cells; and SC-F4 refers to CD55+ lining fibroblasts.
“1” indicates the gene is enriched in the cells; “0” indicates the gene is not enriched in the cells.

TABLE 13
Primers for amplifying the
markers in Table 12

| | PRIMER | SEQ | |
|---|---|---|---|
| GENE | PAIRS: | ID | |
| COL14A1 | Forward | 1 | CACAA |
| (NM_021110.4) | Sequence 1 | | ACCTC |
| | | | CTCAG |
| | | | CGGAA |
| | | | TG |
| | Reverse | 2 | GGCTT |
| | Sequence 1 | | GGAGA |
| | | | TTGGT |
| | | | AACAC |
| | | | CC |
| COL14A1 | Forward | 3 | ATACT |
| (NM_021110.4) | Sequence 2 | | CCGAG |
| | | | GGAAG |
| | | | AGAGC |
| | | | A |
| | Reverse | 4 | CAACC |
| | Sequence 2 | | AGTAC |
| | | | CGCAT |
| | | | CTTGC |
| DCLK1 | Forward | 5 | ACCGA |
| (NM_ | Sequence 1 | | TGCCA |
| 001330071.2) | | | TCAAG |
| | | | CTGGA |
| | | | CT |
| | Reverse | 6 | TCCTG |
| | Sequence 1 | | GTAAC |
| | | | GGAAC |
| | | | TTCTC |
| | | | CG |
| DCLK1 | Forward | 7 | GCATT |
| (NM_001330071.2) | Sequence 2 | | TCAAT |
| | | | GAGGA |
| | | | CGGGC |
| | Reverse | 8 | TACAG |
| | Sequence 2 | | GCGTT |
| | | | TCACC |
| | | | ACTCC |
| FNDC1 | Forward | 9 | TGCAT |
| (NM_032532) | Sequence 1 | | CTTGG |
| | | | GATGC |
| | | | GCTAC |
| | | | CA |
| | Reverse | 10 | GGCAG |
| | Sequence 1 | | AAGTA |
| | | | GTGTC |
| | | | TCCAG |
| | | | GA |
| FNDC1 | Forward | 11 | GATGC |
| (NM_032532) | Sequence 2 | | TACCA |
| | | | GTAGA |
| | | | CCTGT |
| | | | G |
| | Reverse | 12 | GGCAC |
| | Sequence 2 | | TTCCT |
| | | | TTTCT |
| | | | GTGAC |
| | | | G |
| COL16A1 | Forward | 13 | AGGCT |
| (NM_001856.4) | Sequence 1 | | ATGGC |
| | | | AAGAT |
| | | | GGGTG |
| | Reverse | 14 | TGTTC |
| | Sequence 1 | | CTGGG |
| | | | ACTAA |
| | | | ACGGG |
| COL16A1 | Forward | 15 | AACAG |
| (NM_001856.4) | Sequence 2 | | TGAGG |
| | | | GAGAT |
| | | | CCTGG |
| | | | CT |
| | Reverse | 16 | CAACA |
| | Sequence 2 | | GCACC |
| | | | AGGAA |
| | | | AACCT |
| | | | GG |
| COL1A2 | Forward | 17 | CCTCT |
| (NM_000089.4) | Sequence 1 | | GGAGA |
| | | | GGCTG |
| | | | GTACT |
| | Reverse | 18 | TAACC |
| | Sequence 1 | | ACCAC |
| | | | CGCTT |
| | | | ACACC |
| COL1A2 | Forward | 19 | CCTGG |
| (NM_000089.4) | Sequence 2 | | TGCTA |
| | | | AAGGA |
| | | | GAAAG |
| | | | AGG |
| | Reverse | 20 | ATCAC |
| | Sequence 2 | | CACGA |
| | | | CTTCC |
| | | | AGCAG |
| | | | GA |
| KIAA1755 | Forward | 21 | GGTGT |
| (NM_001348708.2) | Sequence | | CAAGG |
| | | | TTTTC |
| | | | CGCTC |
| | | | CA |
| | Reverse | 22 | CTTGG |
| | Sequence | | TGACC |
| | | | TCTGA |
| | | | AACTG |
| | | | TGC |
| PXDN | Forward | 23 | CGGAC |
| (NM_012293.3) | Sequence | | ATTGC |
| | | | AGCTC |
| | | | ATTCA |
| | | | GG |
| | Reverse | 24 | CCGAC |
| | Sequence | | AGGTT |
| | | | TGCGA |
| | | | TGAGG |
| | | | TT |
| COL5A1 | Forward | 25 | CAAAG |
| (NM_000093.5) | Sequence 1 | | AAAAC |
| | | | CCGGG |
| | | | CTCCT |
| | | | G |
| | Reverse | 26 | TGTGA |
| | Sequence 1 | | CGCTT |
| | | | CACCG |
| | | | AAGTC |
| COL5A1 | Forward | 27 | GAATT |
| (NM_000093.5) | Sequence 2 | | CAAGC |
| | | | GTGGG |
| | | | AAACT |
| | | | G |
| | Reverse | 28 | ACTTT |
| | Sequence 2 | | GGGGG |
| | | | TGTCG |
| | | | ATCTC |

This invention may be embodied in other forms or carried out in other ways without departing from the spirit or essential characteristics thereof. The present disclosure is therefore to be considered as in all aspects illustrated and not restrictive, the scope of the present disclosure being indicated by the appended Claims, and all changes which come within the meaning and range of equivalency are intended to be embraced therein.

Various references are cited throughout this Specification, each of which is incorporated herein by reference in its entirety.

What is claimed is:

1. A method for monitoring and/or predicting a rheumatoid arthritis (RA) flare or increased RA disease activity in a patient comprising:
(a) detecting in a blood sample increased amounts of a panel of antecedent RA markers, wherein the panel comprise one or more AC3 markers listed in Table 10 or Table 11;
(b) wherein the expression or quantitatively increased amounts of the AC3 markers predicts an impending RA flare or increased RA disease activity.

2. The method of claim 1, wherein the one or more or all AC3 markers are selected from those listed in Table 12 (COL14A1, DCLK1, FNDC1, COL16A1, COL1A2, KIAA1755, PXDN, and COL5A1).

3. The method of claim 1, wherein a panel of antecedent RA markers comprising at least 2 or more markers listed in Table 12 (COL14A1, DCLK1, FNDC1, COL16A1, COL1A2, KIAA1755, PXDN, and COL5A1) are evaluated to detect increased amounts of one or more of the AC3 markers.

4. The method of claim 1, wherein a panel of antecedent RA markers comprising COL14A1, DCLK1, FNDC1, COL16A1, COL1A2, KIAA1755, PXDN, and COL5A1 are evaluated to detect increased amounts of one or more of the AC3 markers.

5. The method of claim 1, wherein the increased amounts of the AC3 RNA markers or the AC3 protein markers predicts an RA flare in about 1 week or about 5-7 days or up to 2 weeks.

6. The method of claim 1, wherein the panel consists of 2 to 283 antecedent markers.

7. The method of claim 1, wherein a panel of at least 3, at least 4, at least 5 or at least 6 of the AC3 markers are evaluated.

8. The method of claim 1, wherein the method further comprises detecting increased amounts of one or more AC2 markers listed in Table 7.

9. The method of claim 1, wherein the increased amounts of one or more AC3 RNA markers are detected using RNAseq or RT-PCR.

10. The method of claim 9, wherein the detecting the increased amounts of the one or more AC3 RNA markers comprises amplifying the one or more AC3 RNA markers in Table 12 using primer pairs listed in Table 13.

11. The method of claim 1, wherein the increased amount of the one or more AC3 protein markers is detected using antibodies specific for the AC3 protein markers.

12. The method of claim 1 wherein the amount of antecedent AC3 markers are decreased in peripheral blood during an RA flare or once a patient exhibits symptoms of an RA flare.

13. A method for predicting or treating an impending RA flare in a patient, the method comprising:
a) contacting a blood sample from the patient with reagents specific for detecting a panel of AC3 markers, wherein the panel comprise one or more AC3 markers listed in Table 10 or Table 11,
b) detecting amounts of the markers of the panel in the blood sample, wherein detection of increased amounts serves to predict an impending RA flare in a patient,
c) comparing the amounts of the markers in the panel to the amounts of the markers in a control blood sample, and
d) administering a therapeutically effective amount of one or more disease-modifying agent for treating RA if the amounts of the markers of the panel in the blood sample is increased relative to the amounts of the markers in the control blood sample, thereby treating the impending flare in the patient.

14. The method of claim 13, wherein the one or more or all AC3 markers are selected from those listed in Table 12 (COL14A1, DCLK1, FNDC1, COL16A1, COL1A2, KIAA1755, PXDN, and COL5A1).

15. The method of claim 13, wherein a panel of antecedent RA markers comprising at least 2 or more markers AC3 markers listed in Table 12 (COL14A1, DCLK1, FNDC1, COL16A1, COL1A2, KIAA1755, PXDN, and COL5A1) are evaluated to detect increased amounts of one or more of the AC3 markers.

16. The method of claim 13, wherein a panel of antecedent RA markers comprising those AC3 markers listed in Table 12 (COL14A1, DCLK1, FNDC1, COL16A1, COL1A2, KIAA1755, PXDN, and COL5A1) are evaluated to detect increased amounts of one or more of the AC3 markers.

17. The method of claim 13, wherein the increased amounts of the AC3 markers predicts an RA flare in about one week or about 5-7 days.

18. The method of claim 13, wherein step (d) is performed within one (1) week or within 5-7 days from the step (a).

19. The method of claim 13, wherein the disease-modifying agent for treating RA is one or more agent selected from a nonsteroidal anti-inflammatory drug (NSAID), steroid, methotrexate, disease-modifying antirheumatic drug (DMARDs), biologic DMARD, and oral janus kinase (JAK) inhibitor.

20. The method of claim 19, wherein the DMARD is selected from methotrexate (Trexall, Otrexup), leflunomide (Arava), hydroxychloroquine (Plaquenil) and sulfasalazine (Azulfidine).

21. The method of claim 20, wherein the biologic DMARD is selected from abatacept (Orencia), adalimumab (Humira), anakinra (Kineret), baricitinib (Olumiant), certolizumab (Cimzia), etanercept (Enbrel), golimumab (Simponi), infliximab (Remicade), rituximab (Rituxan), sarilumab (Kevzara), tocilizumab (Actemra) and tofacitinib (Xeljanz).

22. The method of claim 20, wherein the biologic DMARD is a tumor necrosis factor (TNF) inhibitor.

23. The method of claim 20, wherein the biologic DMARD is combined with an NSAID and/or with methotrexate.

24. The method of claim 20, wherein the JAK inhibitor is selected from tofacitinib (Xeljanz and Xeljanz XR), baricitinib (Olumiant), and upadacitinib (Rinvoq).

25. The method of claim 13, wherein the disease-modifying agent for treating RA is an IL-17 antibody or an IL-17RD blocking antibody.

26. A method of treating a patient having an impending RA flare or increased RA disease activity, the method comprising
(a) selecting a patient who has been diagnosed as having increased amounts of a panel of markers as compared to a control blood sample,
wherein the panel of markers comprise one or more AC3 markers as listed in Table 10, Table 11, Table 12, and/or one or more AC2 markers listed in Table 7, and
(b) administering to the patient a therapeutically effective amount of one or more disease-modifying agents prior to the onset of RA.

27. A panel of AC3 markers for evaluating and predicting an impending RA flare or increased RA disease activity in a patient comprising the markers selected from one or more antecedent RNA markers or protein markers listed in Table 10, Table 11, or Table 12.

28. A collection of primer pairs for amplifying the AC3 markers in claim 27.

29. A system or kit for predicting an impending RA flare or increased RA disease activity comprising a set of markers of claim 27, or a set of primers and/or antibodies for evaluating a set of markers of claim 27.

30. The system or kit of claim 29, which further comprises a means for collecting the patient's blood by fingerstick.


SEQUENCE LISTING (28 sequences)

SEQ ID NO: 1 - DNA, 22 bp, synthetic construct
  misc_feature 1..22: note = Description of sequence: COL14A1 (NM_021110.4) -Forward Sequence 1
  source 1..22: mol_type = other DNA
cacaaacctcctcagcggaatg

SEQ ID NO: 2 - DNA, 22 bp, synthetic construct
  misc_feature 1..22: note = Description of sequence: COL14A1 (NM_021110.4) -Reverse Sequence 1
  source 1..22: mol_type = other DNA
ggcttggagattggtaacaccc

SEQ ID NO: 3 - DNA, 21 bp, synthetic construct
  misc_feature 1..21: note = Description of sequence: COL14A1 (NM_021110.4) -Forward Sequence 2
  source 1..21: mol_type = other DNA
atactccgagggaagagagca

SEQ ID NO: 4 - DNA, 20 bp, synthetic construct
  misc_feature 1..20: note = Description of sequence: COL14A1 (NM_021110.4) -Reverse Sequence 2
  source 1..20: mol_type = other DNA
caaccagtaccgcatcttgc

SEQ ID NO: 5 - DNA, 22 bp, synthetic construct
  misc_feature 1..22: note = Description of sequence: DCLK1 (NM_001330071.2) -Forward Sequence 1
  source 1..22: mol_type = other DNA
accgatgccatcaagctggact

SEQ ID NO: 6 - DNA, 22 bp, synthetic construct
  misc_feature 1..22: note = Description of sequence: DCLK1 (NM_001330071.2) -Reverse Sequence 1
  source 1..22: mol_type = other DNA
tcctggtaacggaacttctccg

SEQ ID NO: 7 - DNA, 20 bp, synthetic construct
  misc_feature 1..20: note = Description of sequence: DCLK1 (NM_001330071.2) -Forward Sequence 2
  source 1..20: mol_type = other DNA
gcatttcaatgaggacgggc

SEQ ID NO: 8 - DNA, 20 bp, synthetic construct
  misc_feature 1..20: note = Description of sequence: DCLK1 (NM_001330071.2) -Reverse Sequence 2
  source 1..20: mol_type = other DNA
tacaggcgtttcaccactcc

SEQ ID NO: 9 - DNA, 22 bp, synthetic construct
  misc_feature 1..22: note = Description of sequence: FNDC1 (NM_032532)-Forward Sequence 1
  source 1..22: mol_type = other DNA
tgcatcttgggatgcgctacca

SEQ ID NO: 10 - DNA, 22 bp, synthetic construct
  misc_feature 1..22: note = Description of sequence: FNDC1 (NM_032532)-Reverse Sequence 1
  source 1..22: mol_type = other DNA
ggcagaagtagtgtctccagga

SEQ ID NO: 11 - DNA, 21 bp, synthetic construct
  misc_feature 1..21: note = Description of sequence: FNDC1 (NM_032532)-Forward Sequence 2
  source 1..21: mol_type = other DNA
gatgctaccagtagacctgtg

SEQ ID NO: 12 - DNA, 21 bp, synthetic construct
  misc_feature 1..21: note = Description of sequence: FNDC1 (NM_032532) -Reverse Sequence 2
  source 1..21: mol_type = other DNA
ggcacttccttttctgtgacg

SEQ ID NO: 13 - DNA, 20 bp, synthetic construct
  misc_feature 1..20: note = Description of sequence: COL16A1 (NM_001856.4)-Forward Sequence 1
  source 1..20: mol_type = other DNA
aggctatggcaagatgggtg

SEQ ID NO: 14 - DNA, 20 bp, synthetic construct
  misc_feature 1..20: note = Description of sequence: COL16A1 (NM_001856.4)-Reverse Sequence 1
  source 1..20: mol_type = other DNA
tgttcctgggactaaacggg

SEQ ID NO: 15 - DNA, 22 bp, synthetic construct
  misc_feature 1..22: note = Description of sequence: COL16A1 (NM_001856.4)-Forward Sequence 2
  source 1..22: mol_type = other DNA
aacagtgagggagatcctggct

SEQ ID NO: 16 - DNA, 22 bp, synthetic construct
  misc_feature 1..22: note = Description of sequence: COL16A1 (NM_001856.4) -Reverse Sequence 2
  source 1..22: mol_type = other DNA
caacagcaccaggaaaacctgg

SEQ ID NO: 17 - DNA, 20 bp, synthetic construct
  misc_feature 1..20: note = Description of sequence: COL1A2 (NM_000089.4)-Forward Sequence 1
  source 1..20: mol_type = other DNA
cctctggagaggctggtact

SEQ ID NO: 18 - DNA, 20 bp, synthetic construct
  misc_feature 1..20: note = Description of sequence: COL1A2 (NM_000089.4) -Reverse Sequence 1
  source 1..20: mol_type = other DNA
taaccaccaccgcttacacc

SEQ ID NO: 19 - DNA, 23 bp, synthetic construct
  misc_feature 1..23: note = Description of sequence: COL1A2 (NM_000089.4)-Forward Sequence 2
  source 1..23: mol_type = other DNA
cctggtgctaaaggagaaagagg

SEQ ID NO: 20 - DNA, 22 bp, synthetic construct
  misc_feature 1..22: note = Description of sequence: COL1A2 (NM_000089.4) -Reverse Sequence 2
  source 1..22: mol_type = other DNA
atcaccacgacttccagcagga

SEQ ID NO: 21 - DNA, 22 bp, synthetic construct
  misc_feature 1..22: note = Description of sequence: KIAA1755 (NM_001348708.2)-Forward Sequence
  source 1..22: mol_type = other DNA
ggtgtcaaggttttccgctcca

SEQ ID NO: 22 - DNA, 23 bp, synthetic construct
  misc_feature 1..23: note = Description of sequence: KIAA1755 (NM_001348708.2)-Reverse Sequence
  source 1..23: mol_type = other DNA
cttggtgacctctgaaactgtgc

SEQ ID NO: 23 - DNA, 22 bp, synthetic construct
  misc_feature 1..22: note = Description of sequence: PXDN (NM_012293.3) -Forward Sequence
  source 1..22: mol_type = other DNA
cggacattgcagctcattcagg

SEQ ID NO: 24 - DNA, 22 bp, synthetic construct
  misc_feature 1..22: note = Description of sequence: PXDN (NM_012293.3) -Reverse Sequence
  source 1..22: mol_type = other DNA
ccgacaggtttgcgatgaggtt

SEQ ID NO: 25 - DNA, 21 bp, synthetic construct
  misc_feature 1..21: note = Description of sequence: COL5A1 (NM_000093.5) -Forward Sequence 1
  source 1..21: mol_type = other DNA
caaagaaaacccgggctcctg

SEQ ID NO: 26 - DNA, 20 bp, synthetic construct
  misc_feature 1..20: note = Description of sequence: COL5A1 (NM_000093.5) -Reverse Sequence 1
  source 1..20: mol_type = other DNA
tgtgacgcttcaccgaagtc

SEQ ID NO: 27 - DNA, 21 bp, synthetic construct
  misc_feature 1..21: note = Description of sequence: COL5A1 (NM_000093.5) -Forward Sequence 2
  source 1..21: mol_type = other DNA
gaattcaagcgtgggaaactg

SEQ ID NO: 28 - DNA, 20 bp, synthetic construct
  misc_feature 1..20: note = Description of sequence: COL5A1 (NM_000093.5) -Reverse Sequence 2
  source 1..20: mol_type = other DNA
actttgggggtgtcgatctc